SEQUENCE LISTING (577 sequences)

SEQ ID NO: 1 - DNA, 19 bp, synthetic construct
  source 1..19: mol_type = other DNA
ttgagcgggcccccaccgt

SEQ ID NO: 2 - DNA, 21 bp, synthetic construct
  source 1..21: mol_type = other DNA
cagcgggctcagctgatagca

SEQ ID NO: 3 - DNA, 21 bp, synthetic construct
  source 1..21: mol_type = other DNA
cggatggctaaccaagcggcc

SEQ ID NO: 4 - DNA, 393 bp, synthetic construct
  source 1..393: mol_type = other DNA
atgactcactatcaggccttgcttttggacacggaccgggtccagttcggaccggtggtagccctgaacccggctacgctgctcccactgcctgaggaagggctgcaacacaactgccttgatgggacaggtggcggtggtgtcaccgtcaagttcaagtacaagggtgaggaacttgaagttgatattagcaaaatcaagaaggtttggcgcgttggtaaaatgatatcttttacttatgacgacaacggcaagacaggtagaggggcagtgtctgagaaagacgcccccaaggagctgttgcaaatgttggaaaagtctgggaaaaagtctggcggctcaaaaagaaccgccgacggcagcgaattcgagcccaagaagaagaggaaagtc

SEQ ID NO: 5 - DNA, 66 bp, synthetic construct
  source 1..66: mol_type = other DNA
ggaagcggagctactaacttcagcctgctgaagcaggctggcgacgtggaggagaaccctggacct

SEQ ID NO: 6 - DNA, 45 bp, synthetic construct
  source 1..45: mol_type = other DNA
gggggaggaggttctggaggcggaggctccggaggcggagggtca

SEQ ID NO: 7 - DNA, 15 bp, synthetic construct
  source 1..15: mol_type = other DNA
ggaggtggcgggagc

SEQ ID NO: 8 - DNA, 15 bp, synthetic construct
  source 1..15: mol_type = other DNA
cccgcaccagcgcct

SEQ ID NO: 9 - DNA, 45 bp, synthetic construct
  source 1..45: mol_type = other DNA
gaggcagctgccaaggaagccgctgccaaggaggcggccgcaaag

SEQ ID NO: 10 - DNA, 48 bp, synthetic construct
  source 1..48: mol_type = other DNA
agtgggagcgagacccctgggactagcgagtcagctacacccgaaagc

SEQ ID NO: 11 - DNA, 54 bp, synthetic construct
  source 1..54: mol_type = other DNA
ggggggtcaggtggatccggcggaagtggcggatccggtggatctggcggcagt

SEQ ID NO: 12 - DNA, 15 bp, synthetic construct
  source 1..15: mol_type = other DNA
gaagctgctgctaag

SEQ ID NO: 13 - protein, 22 aa, synthetic construct
GSGATNFSLLKQAGDVEENPGP

SEQ ID NO: 14 - protein, 15 aa, synthetic construct
GGGGSGGGGSGGGGS

SEQ ID NO: 15 - protein, 5 aa, synthetic construct
GGGGS

SEQ ID NO: 16 - protein, 5 aa, synthetic construct
PAPAP

SEQ ID NO: 17 - protein, 15 aa, synthetic construct
EAAAKEAAAKEAAAK

SEQ ID NO: 18 - protein, 16 aa, synthetic construct
SGSETPGTSESATPES

SEQ ID NO: 19 - protein, 18 aa, synthetic construct
GGSGGSGGSGGSGGSGGS

SEQ ID NO: 20 - protein, 5 aa, synthetic construct
EAAAK

SEQ ID NO: 21 - protein, 1363 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLS

SEQ ID NO: 22 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 23 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 24 - protein, 1121 aa, Streptococcus thermophilus
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDF

SEQ ID NO: 25 - protein, 1037 aa, Parvibaculum lavamentivorans
MERIFGFDIGTTSIGFSVIDYSSTQSAGNIQRLGVRIFPEARDPDGTPLNQQRRQKRMMRRQLRRRRIRRKALNETLHEAGFLPAYGSADWPVVMADEPYELRRRGLEEGLSAYEFGRAIYHLAQHRHFKGRELEESDTPDPDVDDEKEAANERAATLKALKNEQTTLGAWLARRPPSDRKRGIHAHRNVVAEEFERLWEVQSKFHPALKSEEMRARISDTIFAQRPVFWRKNTLGECRFMPGEPLCPKGSWLSQQRRMLEKLNNLAIAGGNARPLDAEERDAILSKLQQQASMSWPGVRSALKALYKQRGEPGAEKSLKFNLELGGESKLLGNALEAKLADMFGPDWPAHPRKQEIRHAVHERLWAADYGETPDKKRVIILSEKDRKAHREAAANSFVADFGITGEQAAQLQALKLPTGWEPYSIPALNLFLAELEKGERFGALVNGPDWEGWRRTNFPHRNQPTGEILDKLPSPASKEERERISQLRNPTVVRTQNELRKVVNNLIGLYGKPDRIRIEVGRDVGKSKREREEIQSGIRRNEKQRKKATEDLIKNGIANPSRDDVEKWILWKEGQERCPYTGDQIGFNALFREGRYEVEHIWPRSRSFDNSPRNKTLCRKDVNIEKGNRMPFEAFGHDEDRWSAIQIRLQGMVSAKGGTGMSPGKVKRFLAKTMPEDFAARQLNDTRYAAKQILAQLKRLWPDMGPEAPVKVEAVTGQVTAQLRKLWTLNNILADDGEKTRADHRHHAIDALTVACTHPGMTNKLSRYWQLRDDPRAEKPALTPPWDTIRADAEKAVSEIVVSHRVRKKVSGPLHKETTYGDTGTDIKTKSGTYRQFVTRKKIESLSKGELDEIRDPRIKEIVAAHVAGRGGDPKKAFPPYPCVSPGGPEIRKVRLTSKQQLNLMAQTGNGYADLGSNHHIAIYRLPDGKADFEIVSLFDASRRLAQRNPIVQRTRADGASFVMSLAAGEAIMIPEGSKKGIWIVQGVWASGQVVLERDTDADHSTTTRPMPNPILKDDAKKVSIDPIGRVRPSND

SEQ ID NO: 26 - protein, 1084 aa, Corynebacterium diphtheriae
MKYHVGIDVGTFSVGLAAIEVDDAGMPIKTLSLVSHIHDSGLDPDEIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDGPSDAFKAIREEIKRASGQPVPETATVGQMVTLCELGTLKLRGEGGVLSARLQQSDYAREIQEICRMQEIGQELYRKIIDVVFAAESPKGSASSRVGKDPLQPGKNRALKASDAFQRYRIAALIGNLRVRVDGEKRILSVEEKNLVFDHLVNLTPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLSDGVDLYTARLQEFGIEPSWTPPTPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDHIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLKFFDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHKETIGKLSKVKLSSQLSVSDIDKASSEALWCALTREPGFDPKEGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFSPSKLRLRPLQMSKEGIKKESAPELSKIIDRPGWLPAVNKLFSDGNVTVVRRDSLGRVRLESTAHLPVTWKVQ

SEQ ID NO: 27 - protein, 1130 aa, unidentified
MTNGKILGLDIGIASVGVGIIEAKTGKVVHANSRLFSAANAENNAERRGFRGSRRLNRRKKHRVKRVRDLFEKYGIVTDFRNLNLNPYELRVKGLTEQLKNEELFAALRTISKRRGISYLDDAEDDSTGSTDYAKSIDENRRLLKNKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCNFYPDEYRASKASYTAQEYNFLNDLNNLKVSTETGKLSTEQKESLVEFAKNTATLGPAKLLKEIAKILDCKVDEIKGYREDDKGKPDLHTFEPYRKLKFNLESINIDDLSREVIDKLADILTLNTEREGIEDAIKRNLPNQFTEEQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNSSDKLPDEVFHGNKQLETKIRLWYQQGERCLYSGKPISIQELVHNSNNFEIDHILPLSLSFDDSLANKVLVYAWTNQEKGQKTPYQVIDSMDAAWSFREMKDYVLKQKGLGKKKRDYLLTTENIDKIEVKKKFIERNLVDTRYASRVVLNSLQSALRELGKDTKVSVVRGQFTSQLRRKWKIDKSRETYHHHAVDALIIAASSQLKLWEKQDNPMFVDYGKNQVVDKQTGEILSVSDDEYKELVFQPPYQGFVNTISSKGFEDEILFSYQVDSKYNRKVSDATIYSTRKAKIGKDKKEETYVLGKIKDIYSQNGFDTFIKKYNKDKTQFLMYQKDSLTWENVIEVILRDYPTTKKSEDGKNDVKCNPFEEYRRENGLICKYSKKGKGTPIKSLKYYDKKLGNCIDITPEESRNKVILQSINPWRADVYFNPETLKYELMGLKYSDLSFEKGTGNYHISQEKYDAIKEKEGIGKKSEFKFTLYRNDLILIKDIASGEQEIYRFLSRTMPNVNHYVELKPYDKEKFDNVQELVEALGEADKVGRCIKGLNKPNISIYKVRTDVLGNKYFVKKKGDKPKLDFKNNKK

SEQ ID NO: 28 - protein, 1082 aa, Neisseria cinerea
MAAFKPNPMNYILGLDIGIASVGWAIVEIDEEENPIRLIDLGVRVFERAEVPKTGDSLAAARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNTHALQTGDFRTPAELALNKFEKESGHIRNQRGDYSHTFNRKDLQAELNLLFEKQKEFGNPHVSDGLKEGIETLLMTQRPALSGDAVQKMLGHCTFEPTEPKAAKNTYTAERFVWLTKLNNLRILEQGSERPLTDTERATLMDEPYRKSKLTYAQARKLLDLDDTAFFKGLRYGKDNAEASTLMEMKAYHAISRALEKEGLKDKKSPLNLSPELQDEIGTAFSLFKTDEDITGRLKDRVQPEILEALLKHISFDKFVQISLKALRRIVPLMEQGNRYDEACTEIYGDHYGKKNTEEKIYLPPIPADEIRNPVVLRALSQARKVINGVVRRYGSPARIHIETAREVGKSFKDRKEIEKRQEENRKDREKSAAKFREYFPNFVGEPKSKDILKLRLYEQQHGKCLYSGKEINLGRLNEKGYVEIDHALPFSRTWDDSFNNKVLALGSENQNKGNQTPYEYFNGKDNSREWQEFKARVETSRFPRSKKQRILLQKFDEDGFKERNLNDTRYINRFLCQFVADHMLLTGKGKRRVFASNGQITNLLRGFWGLRKVRAENDRHHALDAVVVACSTIAMQQKITRFVRYKEMNAFDGKTIDKETGEVLHQKAHFPQPWEFFAQEVMIRVFGKPDGKPEFEEADTPEKLRTLLAEKLSSRPEAVHKYVTPLFISRAPNRKMSGQGHMETVKSAKRLDEGISVLRVPLTQLKLKDLEKMVNREREPKLYEALKARLEAHKDDPAKAFAEPFYKYDKAGNRTQQVKAVRVEQVQKTGVWVHNHNGIADNATIVRVDVFEKGGKYYLVPIYSWQVAKGILPDRAVVQGKDEEDWTVMDDSFEFKFVLYANDLIKLTAKKNEFLGYFVSLNRATGAIDIRTHDTDSTKGKNGIFQSVGVKTALSFQKYQIDELGKEIRPCRLKKRPPVR

SEQ ID NO: 29 - protein, 1115 aa, unidentified
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 30 - protein, 1003 aa, Campylobacter lari
MRILGFDIGINSIGWAFVENDELKDCGVRIFTKAENPKNKESLALPRRNARSSRRRLKRRKARLIAIKRILAKELKLNYKDYVAADGELPKAYEGSLASVYELRYKALTQNLETKDLARVILHIAKHRGYMNKNEKKSNDAKKGKILSALKNNALKLENYQSVGEYFYKEFFQKYKKNTKNFIKIRNTKDNYNNCVLSSDLEKELKLILEKQKEFGYNYSEDFINEILKVAFFQRPLKDFSHLVGACTFFEEEKRACKNSYSAWEFVALTKIINEIKSLEKISGEIVPTQTINEVLNLILDKGSITYKKFRSCINLHESISFKSLKYDKENAENAKLIDFRKLVEFKKALGVHSLSRQELDQISTHITLIKDNVKLKTVLEKYNLSNEQINNLLEIEFNDYINLSFKALGMILPLMREGKRYDEACEIANLKPKTVDEKKDFLPAFCDSIFAHELSNPVVNRAISEYRKVLNALLKKYGKVHKIHLELARDVGLSKKAREKIEKEQKENQAVNAWALKECENIGLKASAKNILKLKLWKEQKEICIYSGNKISIEHLKDEKALEVDHIYPYSRSFDDSFINKVLVFTKENQEKLNKTPFEAFGKNIEKWSKIQTLAQNLPYKKKNKILDENFKDKQQEDFISRNLNDTRYIATLIAKYTKEYLNFLLLSENENANLKSGEKGSKIHVQTISGMLTSVLRHTWGFDKKDRNNHLHHALDAIIVAYSTNSIIKAFSDFRKNQELLKARFYAKELTSDNYKHQVKFFEPFKSFREKILSKIDEIFVSKPPRKRARRALHKDTFHSENKIIDKCSYNSKEGLQIALSCGRVRKIGTKYVENDTIVRVDIFKKQNKFYAIPIYAMDFALGILPNKIVITGKDKNNNPKQWQTIDESYEFCFSLYKNDLILLQKKNMQEPEFAYYNDFSISTSSICVEKHDNKFENLTSNQKLLFSNAKEGSVKVESLGIQNLKVFEKYIITPLGDKIKADFQPRENISLKTSKKYGLR

SEQ ID NO: 31 - protein, 1374 aa, unidentified
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDF

SEQ ID NO: 32 - protein, 1368 aa, unidentified
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIAMARENQTTQKGQKNSRERMKRIEEGIKELGSQILKEHPVENTQLQNEKLYLYYLQNGRDMYVDQELDINRLSDYDVDAIVPQSFLKDDSIDAKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHAAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 33 - protein, 1422 aa, Sutterella wadsworthensis
MTQSERRFSCSIGIDMGAKYTGVFYALFDREELPTNLNSKAMTLVMPETGPRYVQAQRTAVRHRLRGQKRYTLARKLAFLVVDDMIKKQEKRLTDEEWKRGREALSGLLKRRGYSRPNADGEDLTPLENVRADVFAAHPAFSTYFSEVRSLAEQWEEFTANISNVEKFLGDPNIPADKEFIEFAVAEGLIDKTEKKAYQSALSTLRANANVLTGLRQMGHKPRSEYFKAIEADLKKDSRLAKINEAFGGAERLARLLGNLSNLQLRAERWYFNAPDIMKDRGWEPDRFKKTLVRAFKFFHPAKDQNKQHLELIKQIENSEDIIETLCTLDPNRTIPPYEDQNNRRPPLDQTLLLSPEKLTRQYGEIWKTWSARLTSAEPTLAPAAEILERSTDRKSRVAVNGHEPLPTLAYQLSYALQRAFDRSKALDPYALRALAAGSKSNKLTSARTALENCIGGQNVKTFLDCARRYYREADDAKVGLWFDNADGLLERSDLHPPMKKKILPLLVANILQTDETTGQKFLDEIWRKQIKGRETVASRCARIETVRKSFGGGFNIAYNTAQYREVNKLPRNAQDKELLTIRDRVAETADFIAANLGLSDEQKRKFANPFSLAQFYTLIETEVSGFSATTLAVHLENAWRMTIKDAVINGETVRAAQCSRLPAETARPFDGLVRRLVDRQAWEIAKRVSTDIQSKVDFSNGIVDVSIFVEENKFEFSASVADLKKNKRVKDKMLSEAEKLETRWLIKNERIKKASRGTCPYTGDRLAEGGEIDHILPRSLIKDARGIVFNAEPNLIYASSRGNQLKKNQRYSLSDLKANYRNEIFKTSNIAAITAEIEDVVTKLQQTHRLKFFDLLNEHEQDCVRHALFLDDGSEARDAVLELLATQRRTRVNGTQIWMIKNLANKIREELQNWCKTTNNRLHFQAAATNVSDAKNLRLKLAQNQPDFEKPDIQPIASHSIDALCSFAVGSADAERDQNGFDYLDGKTVLGLYPQSCEVIHLQAKPQEEKSHFDSVAIFKEGIYAEQFLPIFTLNEKIWIGYETLNAKGERCGAIEVSGKQPKELLEMLAPFFNKPVGDLSAHATYRILKKPAYEFLAKAALQPLSAEEKRLAALLDALRYCTSRKSLMSLFMAANGKSLKKREDVLKPKLFQLKVELKGEKSFKLNGSLTLPVKQDWLRICDSPELADAFGKPCSADELTSKLARIWKRPVMRDLAHAPVRREFSLPAIDNPSGGFRIRRTNLFGNELYQVHAINAKKYRGFASAGSNVDWSKGILFNELQHENLTECGGRFITSADVTPMSEWRKVVAEDNLSIWIAPGTEGRRYVRVETTFIQASHWFEQSVENWAITSPLSLPASFKVDKPAEFQKAVGTELSELLGQPRSEIFIENVGNAKHIRFWYIVVSSNKKMNESYNNVSKS

SEQ ID NO: 34 - protein, 1372 aa, Legionella pneumophila
MESSQILSPIGIDLGGKFTGVCLSHLEAFAELPNHANTKYSVILIDHNNFQLSQAQRRATRHRVRNKKRNQFVKRVALQLFQHILSRDLNAKEETALCHYLNNRGYTYVDTDLDEYIKDETTINLLKELLPSESEHNFIDWFLQKMQSSEFRKILVSKVEEKKDDKELKNAVKNIKNFITGFEKNSVEGHRHRKVYFENIKSDITKDNQLDSIKKKIPSVCLSNLLGHLSNLQWKNLHRYLAKNPKQFDEQTFGNEFLRMLKNFRHLKGSQESLAVRNLIQQLEQSQDYISILEKTPPEITIPPYEARTNTGMEKDQSLLLNPEKLNNLYPNWRNLIPGIIDAHPFLEKDLEHTKLRDRKRIISPSKQDEKRDSYILQRYLDLNKKIDKFKIKKQLSFLGQGKQLPANLIETQKEMETHFNSSLVSVLIQIASAYNKEREDAAQGIWFDNAFSLCELSNINPPRKQKILPLLVGAILSEDFINNKDKWAKFKIFWNTHKIGRTSLKSKCKEIEEARKNSGNAFKIDYEEALNHPEHSNNKALIKIIQTIPDIIQAIQSHLGHNDSQALIYHNPFSLSQLYTILETKRDGFHKNCVAVTCENYWRSQKTEIDPEISYASRLPADSVRPFDGVLARMMQRLAYEIAMAKWEQIKHIPDNSSLLIPIYLEQNRFEFEESFKKIKGSSSDKTLEQAIEKQNIQWEEKFQRIINASMNICPYKGASIGGQGEIDHIYPRSLSKKHFGVIFNSEVNLIYCSSQGNREKKEEHYLLEHLSPLYLKHQFGTDNVSDIKNFISQNVANIKKYISFHLLTPEQQKAARHALFLDYDDEAFKTITKFLMSQQKARVNGTQKFLGKQIMEFLSTLADSKQLQLEFSIKQITAEEVHDHRELLSKQEPKLVKSRQQSFPSHAIDATLTMSIGLKEFPQFSQELDNSWFINHLMPDEVHLNPVRSKEKYNKPNISSTPLFKDSLYAERFIPVWVKGETFAIGFSEKDLFEIKPSNKEKLFTLLKTYSTKNPGESLQELQAKSKAKWLYFPINKTLALEFLHHYFHKEIVTPDDTTVCHFINSLRYYTKKESITVKILKEPMPVLSVKFESSKKNVLGSFKHTIALPATKDWERLFNHPNFLALKANPAPNPKEFNEFIRKYFLSDNNPNSDIPNNGHNIKPQKHKAVRKVFSLPVIPGNAGTMMRIRRKDNKGQPLYQLQTIDDTPSMGIQINEDRLVKQEVLMDAYKTRNLSTIDGINNSEGQAYATFDNWLTLPVSTFKPEIIKLEMKPHSKTRRYIRITQSLADFIKTIDEALMIKPSDSIDDPLNMPNEIVCKNKLFGNELKPRDGKMKIVSTGKIVTYEFESDSTPQWIQTLYVTQLKKQP

SEQ ID NO: 35 - protein, 1395 aa, Treponema denticola
MKKEIKDYFLGLDVGTGSVGWAVTDTDYKLLKANRKDLWGMRCFETAETAEVRRLHRGARRRIERRKKRIKLLQELFSQEIAKTDEGFFQRMKESPFYAEDKTILQENTLFNDKDFADKTYHKAYPTINHLIKAWIENKVKPDPRLLYLACHNIIKKRGHFLFEGDFDSENQFDTSIQALFEYLREDMEVDIDADSQKVKEILKDSSLKNSEKQSRLNKILGLKPSDKQKKAITNLISGNKINFADLYDNPDLKDAEKNSISFSKDDFDALSDDLASILGDSFELLLKAKAVYNCSVLSKVIGDEQYLSFAKVKIYEKHKTDLTKLKNVIKKHFPKDYKKVFGYNKNEKNNNNYSGYVGVCKTKSKKLIINNSVNQEDFYKFLKTILSAKSEIKEVNDILTEIETGTFLPKQISKSNAEIPYQLRKMELEKILSNAEKHFSFLKQKDEKGLSHSEKIIMLLTFKIPYYIGPINDNHKKFFPDRCWVVKKEKSPSGKTTPWNFFDHIDKEKTAEAFITSRTNFCTYLVGESVLPKSSLLYSEYTVLNEINNLQIIIDGKNICDIKLKQKIYEDLFKKYKKITQKQISTFIKHEGICNKTDEVIILGIDKECTSSLKSYIELKNIFGKQVDEISTKNMLEEIIRWATIYDEGEGKTILKTKIKAEYGKYCSDEQIKKILNLKFSGWGRLSRKFLETVTSEMPGFSEPVNIITAMRETQNNLMELLSSEFTFTENIKKINSGFEDAEKQFSYDGLVKPLFLSPSVKKMLWQTLKLVKEISHITQAPPKKIFIEMAKGAELEPARTKTRLKILQDLYNNCKNDADAFSSEIKDLSGKIENEDNLRLRSDKLYLYYTQLGKCMYCGKPIEIGHVFDTSNYDIDHIYPQSKIKDDSISNRVLVCSSCNKNKEDKYPLKSEIQSKQRGFWNFLQRNNFISLEKLNRLTRATPISDDETAKFIARQLVETRQATKVAAKVLEKMFPETKIVYSKAETVSMFRNKFDIVKCREINDFHHAHDAYLNIVVGNVYNTKFTNNPWNFIKEKRDNPKIADTYNYYKVFDYDVKRNNITAWEKGKTIITVKDMLKRNTPIYTRQAACKKGELFNQTIMKKGLGQHPLKKEGPFSNISKYGGYNKVSAAYYTLIEYEEKGNKIRSLETIPLYLVKDIQKDQDVLKSYLTDLLGKKEFKILVPKIKINSLLKINGFPCHITGKTNDSFLLRPAVQFCCSNNEVLYFKKIIRFSEIRSQREKIGKTISPYEDLSFRSYIKENLWKKTKNDEIGEKEFYDLLQKKNLEIYDMLLTKHKDTIYKKRPNSATIDILVKGKEKFKSLIIENQFEVILEILKLFSATRNVSDLQHIGGSKYSGVAKIGNKISSLDNCILIYQSITGIFEKRIDLLKV

SEQ ID NO: 36 - protein, 1365 aa, Filifactor alocis
MTKEYYLGLDVGTNSVGWAVTDSQYNLCKFKKKDMWGIRLFESANTAKDRRLQRGNRRRLERKKQRIDLLQEIFSPEICKIDPTFFIRLNESRLHLEDKSNDFKYPLFIEKDYSDIEYYKEFPTIFHLRKHLIESEEKQDIRLIYLALHNIIKTRGHFLIDGDLQSAKQLRPILDTFLLSLQEEQNLSVSLSENQKDEYEEILKNRSIAKSEKVKKLKNLFEISDELEKEEKKAQSAVIENFCKFIVGNKGDVCKFLRVSKEELEIDSFSFSEGKYEDDIVKNLEEKVPEKVYLFEQMKAMYDWNILVDILETEEYISFAKVKQYEKHKTNLRLLRDIILKYCTKDEYNRMFNDEKEAGSYTAYVGKLKKNNKKYWIEKKRNPEEFYKSLGKLLDKIEPLKEDLEVLTMMIEECKNHTLLPIQKNKDNGVIPHQVHEVELKKILENAKKYYSFLTETDKDGYSVVQKIESIFRFRIPYYVGPLSTRHQEKGSNVWMVRKPGREDRIYPWNMEEIIDFEKSNENFITRMTNKCTYLIGEDVLPKHSLLYSKYMVLNELNNVKVRGKKLPTSLKQKVFEDLFENKSKVTGKNLLEYLQIQDKDIQIDDLSGFDKDFKTSLKSYLDFKKQIFGEEIEKESIQNMIEDIIKWITIYGNDKEMLKRVIRANYSNQLTEEQMKKITGFQYSGWGNFSKMFLKGISGSDVSTGETFDIITAMWETDNNLMQILSKKFTFMDNVEDFNSGKVGKIDKITYDSTVKEMFLSPENKRAVWQTIQVAEEIKKVMGCEPKKIFIEMARGGEKVKKRTKSRKAQLLELYAACEEDCRELIKEIEDRDERDFNSMKLFLYYTQFGKCMYSGDDIDINELIRGNSKWDRDHIYPQSKIKDDSIDNLVLVNKTYNAKKSNELLSEDIQKKMHSFWLSLLNKKLITKSKYDRLTRKGDFTDEELSGFIARQLVETRQSTKAIADIFKQIYSSEVVYVKSSLVSDFRKKPLNYLKSRRVNDYHHAKDAYLNIVVGNVYNKKFTSNPIQWMKKNRDTNYSLNKVFEHDVVINGEVIWEKCTYHEDTNTYDGGTLDRIRKIVERDNILYTEYAYCEKGELFNATIQNKNGNSTVSLKKGLDVKKYGGYFSANTSYFSLIEFEDKKGDRARHIIGVPIYIANMLEHSPSAFLEYCEQKGYQNVRILVEKIKKNSLLIINGYPLRIRGENEVDTSFKRAIQLKLDQKNYELVRNIEKFLEKYVEKKGNYPIDENRDHITHEKMNQLYEVLLSKMKKFNKKGMADPSDRIEKSKPKFIKLEDLIDKINVINKMLNLLRCDNDTKADLSLIELPKNAGSFVVKKNTIGKSKIILVNQSVTGLYENRREL

SEQ ID NO: 37 - protein, 1334 aa, Staphylococcus pseudintermedius
MGRKPYILSLDIGTGSVGYACMDKGFNVLKYHDKDALGVYLFDGALTAQERRQFRTSRRRKNRRIKRLGLLQELLAPLVQNPNFYQFQRQFAWKNDNMDFKNKSLSEVLSFLGYESKKYPTIYHLQEALLLKDEKFDPELIYMALYHLVKYRGHFLFDHLKIENLTNNDNMHDFVELIETYENLNNIKLNLDYEKTKVIYEILKDNEMTKNDRAKRVKNMEKKLEQFSIMLLGLKFNEGKLFNHADNAEELKGANQSHTFADNYEENLTPFLTVEQSEFIERANKIYLSLTLQDILKGKKSMAMSKVAAYDKFRNELKQVKDIVYKADSTRTQFKKIFVSSKKSLKQYDATPNDQTFSSLCLFDQYLIRPKKQYSLLIKELKKIIPQDSELYFEAENDTLLKVLNTTDNASIPMQINLYEAETILRNQQKYHAEITDEMIEKVLSLIQFRIPYYVGPLVNDHTASKFGWMERKSNESIKPWNFDEVVDRSKSATQFIRRMTNKCSYLINEDVLPKNSLLYQEMEVLNELNATQIRLQTDPKNRKYRMMPQIKLFAVEHIFKKYKTVSHSKFLEIMLNSNHRENFMNHGEKLSIFGTQDDKKFASKLSSYQDMTKIFGDIEGKRAQIEEIIQWITIFEDKKILVQKLKECYPELTSKQINQLKKLNYSGWGRLSEKLLTHAYQGHSIIELLRHSDENFMEILTNDVYGFQNFIKEENQVQSNKIQHQDIANLTTSPALKKGIWSTIKLVRELTSIFGEPEKIIMEFATEDQQKGKKQKSRKQLWDDNIKKNKLKSVDEYKYIIDVANKLNNEQLQQEKLWLYLSQNGKCMYSGQSIDLDALLSPNATKHYEVDHIFPRSFIKDDSIDNKVLVIKKMNQTKGDQVPLQFIQQPYERIAYWKSLNKAGLISDSKLHKLMKPEFTAMDKEGFIQRQLVETRQISVHVRDFLKEEYPNTKVIPMKAKMVSEFRKKFDIPKIRQMNDAHHAIDAYLNGVVYHGAQLAYPNVDLFDFNFKWEKVREKWKALGEFNTKQKSRELFFFKKLEKMEVSQGERLISKIKLDMNHFKINYSRKLANIPQQFYNQTAVSPKTAELKYESNKSNEVVYKGLTPYQTYVVAIKSVNKKGKEKMEYQMIDHYVFDFYKFQNGNEKELALYLAQRENKDEVLDAQIVYSLNKGDLLYINNHPCYFVSRKEVINAKQFELTVEQQLSLYNVMNNKETNVEKLLIEYDFIAEKVINEYHHYLNSKLKEKRVRTFFSESNQTHEDFIKALDELFKVVTASATRSDKIGSRKNSMTHRAFLGKGKDVKIAYTSISGLKTTKPKSLFKLAESRNEL

SEQ ID NO: 38 - protein, 1375 aa, Lactobacillus johnsonii
MTKIKDDYIVGLDIGTDSCGWVAMNSNNDILKLQGKTAIGSRLFEGGKSAAERRLFRTTHRRIKRRRWRLKLLEEFFDPYMAEVDPYFFARLKESGLSPLDKRKTVSSIVFPTSAEDKKFYDDYPTIYHLRYKLMTEDEKFDLREVYLAIHHIIKYRGNFLYNTSVKDFKASKIDVKSSIEKLNELYENLGLDLNVEFNISNTAEIEKVLKDKQIFKRDKVKKIAELFAIKTDNKEQSKRIKDISKQVANAVLGYKTRFDTIALKEISKDELSDWNFKLSDIDADSKFEALMGNLDENEQAILLTIKELFNEVTLNGIVEDGNTLSESMINKYNDHRDDLKLLKEVIENHIDRKKAKELALAYDLYVNNRHGQLLQAKKKLGKIKPRSKEDFYKVVNKNLDDSRASKEIKKKIELDSFMPKQRTNANGVIPYQLQQLELDKIIENQSKYYPFLKEINPVSSHLKEAPYKLDELIRFRVPYYVGPLISPNESTKDIQTKKNQNFAWMIRKEEGRITPWNFDQKVDRIESANKFIKRMTTKDTYLFGEDVLPANSLLYQKFTVLNELNNIRINGKRISVDLKQEIYENLFKKHTTVTVKKLENYLKENHNLVKVEIKGLADEKKFNSGLTTYNRFKNLNIFDNQIDDLKYRNDFEKIIEWSTIFEDKSIYKEKLRSIDWLNEKQINALSNIRLQGWGRLSKKLLAQLHDHNGQTIIEQLWDSQNNFMQIVTQADFKDAIAKANQNLLVATSVEDILNNAYTSPANKKAIRQVIKVVDDIVKAASGKVPKQIAIEFTRDADENPKRSQTRGSKLQKVYKDLSTELASKTIAEELNEAIKDKKLVQDKYYLYFMQLGRDAYTGEPINIDEIQKYDIDHILPQSFIKDDALDNRVLVSRAVNNGKSDNVPVKLFGNEMAANLGMTIRKMWEEWKNIGLISKTKYNNLLTDPDHINKYKSAGFIRRQLVETSQIIKLVSTILQSRYPNTEIITVKAKYNHYLREKFDLYKSREVNDYHHAIDAYLSAICGNLLYQNYPNLRPFFVYGQYKKFSSDPDKEKAIFNKTRKFSFISQLLKNKSENSKEIAKKLKRAYQFKYMLVSRETETRDQEMFKMTVYPRFSHDTVKAPRNLIPKKMGMSPDIYGGYTNNSDAYMVIVRIDKKKGTEYKILGIPTRELVNLKKAEKEDHYKSYLKEILTPRILYNKNGKRDKKITSFEIVKSKIPYKQVIQDGDKKFMLGSSTYVYNAKQLTLSTESMKAITNNFDKDSDENDALIKAYDEILDKVDKYLPLFDINKFREKLHSGREKFIKLSLEDKKDTILKVLEGLHDNAVMTKIPTIGLSTPLGFMQFPNGVILSENAKLIYQSPTGLFKKSVKISDL

SEQ ID NO: 39 - protein, 1269 aa, Mycoplasma gallisepticum
MNNSIKSKPEVTIGLDLGVGSVGWAIVDNETNIIHHLGSRLFSQAKTAEDRRSFRGVRRLIRRRKYKLKRFVNLIWKYNSYFGFKNKEDILNNYQEQQKLHNTVLNLKSEALNAKIDPKALSWILHDYLKNRGHFYEDNRDFNVYPTKELAKYFDKYGYYKGIIDSKEDNDNKLEEELTKYKFSNKHWLEEVKKVLSNQTGLPEKFKEEYESLFSYVRNYSEGPGSINSVSPYGIYHLDEKEGKVVQKYNNIWDKTIGKCNIFPDEYRAPKNSPIAMIFNEINELSTIRSYSIYLTGWFINQEFKKAYLNKLLDLLIKTNGEKPIDARQFKKLREETIAESIGKETLKDVENEEKLEKEDHKWKLKGLKLNTNGKIQYNDLSSLAKFVHKLKQHLKLDFLLEDQYATLDKINFLQSLFVYLGKHLRYSNRVDSANLKEFSDSNKLFERILQKQKDGLFKLFEQTDKDDEKILAQTHSLSTKAMLLAITRMTNLDNDEDNQKNNDKGWNFEAIKNFDQKFIDITKKNNNLSLKQNKRYLDDRFINDAILSPGVKRILREATKVFNAILKQFSEEYDVTKVVIELARELSEEKELENTKNYKKLIKKNGDKISEGLKALGISEDEIKDILKSPTKSYKFLLWLQQDHIDPYSLKEIAFDDIFTKTEKFEIDHIIPYSISFDDSSSNKLLVLAESNQAKSNQTPYEFISSGNAGIKWEDYEAYCRKFKDGDSSLLDSTQRSKKFAKMMKTDTSSKYDIGFLARNLNDTRYATIVFRDALEDYANNHLVEDKPMFKVVCINGSVTSFLRKNFDDSSYAKKDRDKNIHHAVDASIISIFSNETKTLFNQLTQFADYKLFKNTDGSWKKIDPKTGVVTEVTDENWKQIRVRNQVSEIAKVIEKYIQDSNIERKARYSRKIENKTNISLFNDTVYSAKKVGYEDQIKRKNLKTLDIHESAKENKNSKVKRQFVYRKLVNVSLLNNDKLADLFAEKEDILMYRANPWVINLAEQIFNEYTENKKIKSQNVFEKYMLDLTKEFPEKFSEFLVKSMLRNKTAIIYDDKKNIVHRIKRLKMLSSELKENKLSNVIIRSKNQSGTKLSYQDTINSLALMIMRSIDPTAKKQYIRVPLNTLNLHLGDHDFDLHNMDAYLKKPKFVKYLKANEIGDEYKPWRVLTSGTLLIHKKDKKLMYISSFQNLNDVIEIKNLIETEYKENDDSDSKKKKKANRFLMTLSTILNDYILLDAKDNFDILGLSKNRIDEILNSKLGLDKIVK

SEQ ID NO: 40 - protein, 1003 aa, unidentified; identical to SEQ ID NO: 30

SEQ ID NO: 41 - protein, 1120 aa, unidentified
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLM

SEQ ID NO: 42 - protein, 1415 aa, Bergeyella zoohelcum
MKHILGLDLGTNSIGWALIERNIEEKYGKIIGMGSRIVPMGAELSKFEQGQAQTKNADRRTNRGARRLNKRYKQRRNKLIYILQKLDMLPSQIKLKEDFSDPNKIDKITILPISKKQEQLTAFDLVSLRVKALTEKVGLEDLGKIIYKYNQLRGYAGGSLEPEKEDIFDEEQSKDKKNKSFIAFSKIVFLGEPQEEIFKNKKLNRRAIIVETEEGNFEGSTFLENIKVGDSLELLINISASKSGDTITIKLPNKTNWRKKMENIENQLKEKSKEMGREFYISEFLLELLKENRWAKIRNNTILRARYESEFEAIWNEQVKHYPFLENLDKKTLIEIVSFIFPGEKESQKKYRELGLEKGLKYIIKNQVVFYQRELKDQSHLISDCRYEPNEKAIAKSHPVFQEYKVWEQINKLIVNTKIEAGTNRKGEKKYKYIDRPIPTALKEWIFEELQNKKEITFSAIFKKLKAEFDLREGIDFLNGMSPKDKLKGNETKLQLQKSLGELWDVLGLDSINRQIELWNILYNEKGNEYDLTSDRTSKVLEFINKYGNNIVDDNAEETAIRISKIKFARAYSSLSLKAVERILPLVRAGKYFNNDFSQQLQSKILKLLNENVEDPFAKAAQTYLDNNQSVLSEGGVGNSIATILVYDKHTAKEYSHDELYKSYKEINLLKQGDLRNPLVEQIINEALVLIRDIWKNYGIKPNEIRVELARDLKNSAKERATIHKRNKDNQTINNKIKETLVKNKKELSLANIEKVKLWEAQRHLSPYTGQPIPLSDLFDKEKYDVDHIIPISRYFDDSFTNKVISEKSVNQEKANRTAMEYFEVGSLKYSIFTKEQFIAHVNEYFSGVKRKNLLATSIPEDPVQRQIKDTQYIAIRVKEELNKIVGNENVKTTTGSITDYLRNHWGLTDKFKLLLKERYEALLESEKFLEAEYDNYKKDFDSRKKEYEEKEVLFEEQELTREEFIKEYKENYIRYKKNKLIIKGWSKRIDHRHHAIDALIVACTEPAHIKRLNDLNKVLQDWLVEHKSEFMPNFEGSNSELLEEILSLPENERTEIFTQIEKFRAIEMPWKGFPEQVEQKLKEIIISHKPKDKLLLQYNKAGDRQIKLRGQLHEGTLYGISQGKEAYRIPLTKFGGSKFATEKNIQKIVSPFLSGFIANHLKEYNNKKEEAFSAEGIMDLNNKLAQYRNEKGELKPHTPISTVKIYYKDPSKNKKKKDEEDLSLQKLDREKAFNEKLYVKTGDNYLFAVLEGEIKTKKTSQIKRLYDIISFFDATNFLKEEFRNAPDKKTFDKDLLFRQYFEERNKAKLLFTLKQGDFVYLPNENEEVILDKESPLYNQYWGDLKERGKNIYVVQKFSKKQIYFIKHTIADIIKKDVEFGSQNCYETVEGRSIKENCFKLEIDRLGNIVKVIKR

SEQ ID NO: 43 - protein, 1338 aa, Coprococcus catus
MKQEYFLGLDMGTGSLGWAVTDSTYQVMRKHGKALWGTRLFESASTAEERRMFRTARRRLDRRNWRIQVLQEIFSEEISKVDPGFFLRMKESKYYPEDKRDAEGNCPELPYALFVDDNYTDKNYHKDYPTIYHLRKMLMETTEIPDIRLVYLVLHHMMKHRGHFLLSGDISQIKEFKSTFEQLIQNIQDEELEWHISLDDAAIQFVEHVLKDRNLTRSTKKSRLIKQLNAKSACEKAILNLLSGGTVKLSDIFNNKELDESERPKVSFADSGYDDYIGIVEAELAEQYYIIASAKAVYDWSVLVEILGNSVSISEAKIKVYQKHQADLKTLKKIVRQYMTKEDYKRVFVDTEEKLNNYSAYIGMTKKNGKKVDLKSKQCTQADFYDFLKKNVIKVIDHKEITQEIESEIEKENFLPKQVTKDNGVIPYQVHDYELKKILDNLGTRMPFIKENAEKIQQLFEFRIPYYVGPLNRVDDGKDGKFTWSVRKSDARIYPWNFTEVIDVEASAEKFIRRMTNKCTYLVGEDVLPKDSLVYSKFMVLNELNNLRLNGEKISVELKQRIYEELFCKYRKVTRKKLERYLVIEGIAKKGVEITGIDGDFKASLTAYHDFKERLTDVQLSQRAKEAIVLNVVLFGDDKKLLKQRLSKMYPNLTTGQLKGICSLSYQGWGRLSKTFLEEITVPAPGTGEVWNIMTALWQTNDNLMQLLSRNYGFTNEVEEFNTLKKETDLSYKTVDELYVSPAVKRQIWQTLKVVKEIQKVMGNAPKRVFVEMAREKQEGKRSDSRKKQLVELYRACKNEERDWITELNAQSDQQLRSDKLFLYYIQKGRCMYSGETIQLDELWDNTKYDIDHIYPQSKTMDDSLNNRVLVKKNYNAIKSDTYPLSLDIQKKMMSFWKMLQQQGFITKEKYVRLVRSDELSADELAGFIERQIVETRQSTKAVATILKEALPDTEIVYVKAGNVSNFRQTYELLKVREMNDLHHAKDAYLNIVVGNAYFVKFTKNAAWFIRNNPGRSYNLKRMFEFDIERSGEIAWKAGNKGSIVTVKKVMQKNNILVTRKAYEVKGGLFDQQIMKKGKGQVPIKGNDERLADIEKYGGYNKAAGTYFMLVKSLDKKGKEIRTIEFVPLYLKNQIEINHESAIQYLAQERGLNSPEILLSKIKIDTLFKVDGFKMWLSGRTGNQLIFKGANQLILSHQEAAILKGVVKYVNRKNENKDAKLSERDGMTEEKLLQLYDTFLDKLSNTVYSIRLSAQIKTLTEKRAKFIGLSNEDQCIVLNEILHMFQCQSGSANLKLIGGPGSAGILVMNNNITACKQISVINQSPTGIYEKEIDLIKL

SEQ ID NO: 44 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 45 - protein, 1195 aa, Elusimicrobium minutum
MQKNINTKQNHIYIKQAQKIKEKLGDKPYRIGLDLGVGSIGFAIVSMEENDGNVLLPKEIIMVGSRIFKASAGAADRKLSRGQRNNHRHTRERMRYLWKVLAEQKLALPVPADLDRKENSSEGETSAKRFLGDVLQKDIYELRVKSLDERLSLQELGYVLYHIAGHRGSSAIRTFENDSEEAQKENTENKKIAGNIKRLMAKKNYRTYGEYLYKEFFENKEKHKREKISNAANNHKFSPTRDLVIKEAEAILKKQAGKDGFHKELTEEYIEKLTKAIGYESEKLIPESGFCPYLKDEKRLPASHKLNEERRLWETLNNARYSDPIVDIVTGEITGYYEKQFTKEQKQKLFDYLLTGSELTPAQTKKLLGLKNTNFEDIILQGRDKKAQKIKGYKLIKLESMPFWARLSEAQQDSFLYDWNSCPDEKLLTEKLSNEYHLTEEEIDNAFNEIVLSSSYAPLGKSAMLIILEKIKNDLSYTEAVEEALKEGKLTKEKQAIKDRLPYYGAVLQESTQKIIAKGFSPQFKDKGYKTPHTNKYELEYGRIANPVVHQTLNELRKLVNEIIDILGKKPCEIGLETARELKKSAEDRSKLSREQNDNESNRNRIYEIYIRPQQQVIITRRENPRNYILKFELLEEQKSQCPFCGGQISPNDIINNQADIEHLFPIAESEDNGRNNLVISHSACNADKAKRSPWAAFASAAKDSKYDYNRILSNVKENIPHKAWRFNQGAFEKFIENKPMAARFKTDNSYISKVAHKYLACLFEKPNIICVKGSLTAQLRMAWGLQGLMIPFAKQLITEKESESFNKDVNSNKKIRLDNRHHALDAIVIAYASRGYGNLLNKMAGKDYKINYSERNWLSKILLPPNNIVWENIDADLESFESSVKTALKNAFISVKHDHSDNGELVKGTMYKIFYSERGYTLTTYKKLSALKLTDPQKKKTPKDFLETALLKFKGRESEMKNEKIKSAIENNKRLFDVIQDNLEKAKKLLEEENEKSKAEGKKEKNINDASIYQKAISLSGDKYVQLSKKEPGKFFAISKPTPTTTGYGYDTGDSLCVDLYYDNKGKLCGEIIRKIDAQQKNPLKYKEQGFTLFERIYGGDILEVDFDIHSDKNSFRNNTGSAPENRVFIKVGTFTEITNNNIQIWFGNIIKSTGGQDDSFTINSMQQYNPRKLILSSCGFIKYRSPILKNKEG

SEQ ID NO: 46 - protein, 1062 aa, Treponema sp.
MIMKLEKWRLGLDLGTNSIGWSVFSLDKDNSVQDLIDMGVRIFSDGRDPKTKEPLAVARRTARSQRKLIYRRKLRRKQVFKFLQEQGLFPKTKEECMTLKSLNPYELRIKALDEKLEPYELGRALFNLAVRRGFKSNRKDGSREEVSEKKSPDEIKTQADMQTHLEKAIKENGCRTITEFLYKNQGENGGIRFAPGRMTYYPTRKMYEEEFNLIRSKQEKYYPQVDWDDIYKAIFYQRPLKPQQRGYCIYENDKERTFKAMPCSQKLRILQDIGNLAYYEGGSKKRVELNDNQDKVLYELLNSKDKVTFDQMRKALCLADSNSFNLEENRDFLIGNPTAVKMRSKNRFGKLWDEIPLEEQDLIIETIITADEDDAVYEVIKKYDLTQEQRDFIVKNTILQSGTSMLCKEVSEKLVKRLEEIADLKYHEAVESLGYKFADQTVEKYDLLPYYGKVLPGSTMEIDLSAPETNPEKHYGKISNPTVHVALNQTRVVVNALIKEYGKPSQIAIELSRDLKNNVEKKAEIARKQNQRAKENIAINDTISALYHTAFPGKSFYPNRNDRMKYRLWSELGLGNKCIYCGKGISGAELFTKEIEIEHILPFSRTLLDAESNLTVAHSSCNAFKAERSPFEAFGTNPSGYSWQEIIQRANQLKNTSKKNKFSPNAMDSFEKDSSFIARQLSDNQYIAKAALRYLKCLVENPSDVWTTNGSMTKLLRDKWEMDSILCRKFTEKEVALLGLKPEQIGNYKKNRFDHRHHAIDAVVIGLTDRSMVQKLATKNSHKGNRIEIPEFPILRSDLIEKVKNIVVSFKPDHGAEGKLSKETLLGKIKLHGKETFVCRENIVSLSEKNLDDIVDEKIKSKVKDYVAKHKGQKIEAVLSDFSKENGIKKVRCVNRVQTPIEITSGKISRYLSPEDYFAAVIWEIPGEKKTFKAQYIRRNEVEKNSKGLNVVKPAVLENGKPHPAAKQVCLLHKDDYLEFSDKGKMYFCRIAGYAATNNKLDIRPVYAVSYCADWINSTNETMLTGYWKPTPTQNWVSVNVLFDKQKARLVTVSPIGRVFRK

SEQ ID NO: 47 - protein, 1054 aa, Staphylococcus lugdunensis
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 48 - protein, 1388 aa, Streptococcus thermophilus
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 49 - protein, 1436 aa, Bacteroides fragilis
MKRILGLDLGTNSIGWALVNEAENKDERSSIVKLGVRVNPLTVDELTNFEKGKSITTNADRTLKRGMRRNLQRYKLRRETLTEVLKEHKLITEDTILSENGNRTTFETYRLRAKAVTEEISLEEFARVLLMINKKRGYKSSRKAKGVEEGTLIDGMDIARELYNNNLTPGELCLQLLDAGKKFLPDFYRSDLQNELDRIWEKQKEYYPEILTDVLKEELRGKKRDAVWAICAKYFVWKENYTEWNKEKGKTEQQEREHKLEGIYSKRKRDEAKRENLQWRVNGLKEKLSLEQLVIVFQEMNTQINNSSGYLGAISDRSKELYFNKQTVGQYQMEMLDKNPNASLRNMVFYRQDYLDEFNMLWEKQAVYHKELTEELKKEIRDIIIFYQRRLKSQKGLIGFCEFESRQIEVDIDGKKKIKTVGNRVISRSSPLFQEFKIWQILNNIEVTVVGKKRKRRKLKENYSALFEELNDAEQLELNGSRRLCQEEKELLAQELFIRDKMTKSEVLKLLFDNPQELDLNFKTIDGNKTGYALFQAYSKMIEMSGHEPVDFKKPVEKVVEYIKAVFDLLNWNTDILGFNSNEELDNQPYYKLWHLLYSFEGDNTPTGNGRLIQKMTELYGFEKEYATILANVSFQDDYGSLSAKAIHKILPHLKEGNRYDVACVYAGYRHSESSLTREEIANKVLKDRLMLLPKNSLHNPVVEKILNQMVNVINVIIDIYGKPDEIRVELARELKKNAKEREELTKSIAQTTKAHEEYKTLLQTEFGLTNVSRTDILRYKLYKELESCGYKTLYSNTYISREKLFSKEFDIEHIIPQARLFDDSFSNKTLEARSVNIEKGNKTAYDFVKEKFGESGADNSLEHYLNNIEDLFKSGKISKTKYNKLKMAEQDIPDGFIERDLRNTQYIAKKALSMLNEISHRVVATSGSVTDKLREDWQLIDVMKELNWEKYKALGLVEYFEDRDGRQIGRIKDWTKRNDHRHHAMDALTVAFTKDVFIQYFNNKNASLDPNANEHAIKNKYFQNGRAIAPMPLREFRAEAKKHLENTLISIKAKNKVITGNINKTRKKGGVNKNMQQTPRGQLHLETIYGSGKQYLTKEEKVNASFDMRKIGTVSKSAYRDALLKRLYENDNDPKKAFAGKNSLDKQPIWLDKEQMRKVPEKVKIVTLEAIYTIRKEISPDLKVDKVIDVGVRKILIDRLNEYGNDAKKAFSNLDKNPIWLNKEKGISIKRVTISGISNAQSLHVKKDKDGKPILDENGRNIPVDFVNTGNNHHVAVYYRPVIDKRGQLVVDEAGNPKYELEEVVVSFFEAVTRANLGLPIIDKDYKTTEGWQFLFSMKQNEYFVFPNEKTGFNPKEIDLLDVENYGLISPNLFRVQKFSLKNYVFRHHLETTIKDTSSILRGITWIDFRSSKGLDTIVKVRVNHIGQIVSVGEY

SEQ ID NO: 50 - protein, 1398 aa, Veillonella atypica
METQTSNQLITSHLKDYPKQDYFVGLDIGTNSVGWAVTNTSYELLKFHSHKMWGSRLFEEGESAVTRRGFRSMRRRLERRKLRLKLLEELFADAMAQVDSTFFIRLHESKYHYEDKTTGHSSKHILFIDEDYTDQDYFTEYPTIYHLRKDLMENGTDDIRKLFLAVHHILKYRGNFLYEGATFNSNAFTFEDVLKQALVNITFNCFDTNSAISSISNILMESGKTKSDKAKAIERLVDTYTVFDEVNTPDKPQKEQVKEDKKTLKAFANLVLGLSANLIDLFGSVEDIDDDLKKLQIVGDTYDEKRDELAKVWGDEIHIIDDCKSVYDAIILMSIKEPGLTISQSKVKAFDKHKEDLVILKSLLKLDRNVYNEMFKSDKKGLHNYVHYIKQGRTEETSCSREDFYKYTKKIVEGLADSKDKEYILNEIELQTLLPLQRIKDNGVIPYQLHLEELKVILDKCGPKFPFLHTVSDGFSVTEKLIKMLEFRIPYYVGPLNTHHNIDNGGFSWAVRKQAGRVTPWNFEEKIDREKSAAAFIKNLTNKCTYLFGEDVLPKSSLLYSEFMLLNELNNVRIDGKALAQGVKQHLIDSIFKQDHKKMTKNRIELFLKDNNYITKKHKPEITGLDGEIKNDLTSYRDMVRILGNNFDVSMAEDIITDITIFGESKKMLRQTLRNKFGSQLNDETIKKLSKLRYRDWGRLSKKLLKGIDGCDKAGNGAPKTIIELMRNDSYNLMEILGDKFSFMECIEEENAKLAQGQVVNPHDIIDELALSPAVKRAVWQALRIVDEVAHIKKALPSRIFVEVARTNKSEKKKKDSRQKRLSDLYSAIKKDDVLQSGLQDKEFGALKSGLANYDDAALRSKKLYLYYTQMGRCAYTGNIIDLNQLNTDNYDIDHIYPRSLTKDDSFDNLVLCERTANAKKSDIYPIDNRIQTKQKPFWAFLKHQGLISERKYERLTRIAPLTADDLSGFIARQLVETNQSVKATTTLLRRLYPDIDVVFVKAENVSDFRHNNNFIKVRSLNHHHHAKDAYLNIVVGNVYHEKFTRNFRLFFKKNGANRTYNLAKMFNYDVICTNAQDGKAWDVKTSMNTVKKMMASNDVRVTRRLLEQSGALADATIYKASVAAKAKDGAYIGMKTKYSVFADVTKYGGMTKIKNAYSIIVQYTGKKGEEIKEIVPLPIYLINRNATDIELIDYVKSVIPKAKDISIKYRKLCINQLVKVNGFYYYLGGKTNDKIYIDNAIELVVPHDIATYIKLLDKYDLLRKENKTLKASSITTSIYNINTSTVVSLSNKVGIDVFDYFMSKLRTPLYMKMKGNKVDELSSTGRSKFIKMTLEEQSIYLLEVLNLLTNSKTTFDVKPLGITGSRSTIGVKIHNLDEFKIINESITGLYSNEVTIV

SEQ ID NO: 51 - protein, 1092 aa, Ilyobacter polytropus
MKYSIGLDIGIASVGWSVINKDKERIEDMGVRIFQKAENPKDGSSLASSRREKRGSRRRNRRKKHRLDRIKNILCESGLVKKNEIEKIYKNAYLKSPWELRAKSLEAKISNKEIAQILLHIAKRRGFKSFRKTDRNADDTGKLLSGIQENKKIMEEKGYLTIGDMVAKDPKFNTHVRNKAGSYLFSFSRKLLEDEVRKIQAKQKELGNTHFTDDVLEKYIEVFNSQRNFDEGPSKPSPYYSEIGQIAKMIGNCTFESSEKRTAKNTWSGERFVFLQKLNNFRIVGLSGKRPLTEEERDIVEKEVYLKKEVRYEKLRKILYLKEEERFGDLNYSKDEKQDKKTEKTKFISLIGNYTIKKLNLSEKLKSEIEEDKSKLDKIIEILTFNKSDKTIESNLKKLELSREDIEILLSEEFSGTLNLSLKAIKKILPYLEKGLSYNEACEKADYDYKNNGIKFKRGELLPVVDKDLIANPVVLRAISQTRKVVNAIIRKYGTPHTIHVEVARDLAKSYDDRQTIIKENKKRELENEKTKKFISEEFGIKNVKGKLLLKYRLYQEQEGRCAYSRKELSLSEVILDESMTDIDHIIPYSRSMDDSYSNKVLVLSGENRKKSNLLPKEYFDRQGRDWDTFVLNVKAMKIHPRKKSNLLKEKFTREDNKDWKSRALNDTRYISRFVANYLENALEYRDDSPKKRVFMIPGQLTAQLRARWRLNKVRENGDLHHALDAAVVAVTDQKAINNISNISRYKELKNCKDVIPSIEYHADEETGEVYFEEVKDTRFPMPWSGFDLELQKRLESENPREEFYNLLSDKRYLGWFNYEEGFIEKLRPVFVSRMPNRGVKGQAHQETIRSSKKISNQIAVSKKPLNSIKLKDLEKMQGRDTDRKLYEALKNRLEEYDDKPEKAFAEPFYKPTNSGKRGPLVRGIKVEEKQNVGVYVNGGQASNGSMVRIDVFRKNGKFYTVPIYVHQTLLKELPNRAINGKPYKDWDLIDGSFEFLYSFYPNDLIEIEFGKSKSIKNDNKLTKTEIPEVNLSEVLGYYRGMDTSTGAATIDTQDGKIQMRIGIKTVKNIKKYQVDVLGNVYKVKREKRQTF

SEQ ID NO: 52 - protein, 1517 aa, Parabacteroides sp.
MKKIVGLDLGTNSIGWALINAYINKEHLYGIEACGSRIIPMDAAILGNFDKGNSISQTADRTSYRGIRRLRERHLLRRERLHRILDLLGFLPKHYSDSLNRYGKFLNDIECKLPWVKDETGSYKFIFQESFKEMLANFTEHHPILIANNKKVPYDWTIYYLRKKALTQKISKEELAWILLNFNQKRGYYQLRGEEEETPNKLVEYYSLKVEKVEDSGERKGKDTWYNVHLENGMIYRRTSNIPLDWEGKTKEFIVTTDLEADGSPKKDKEGNIKRSFRAPKDDDWTLIKKKTEADIDKIKMTVGAYIYDTLLQKPDQKIRGKLVRTIERKYYKNELYQILKTQSEFHEELRDKQLYIACLNELYPNNEPRRNSISTRDFCHLFIEDIIFYQRPLKSKKSLIDNCPYEENRYIDKESGEIKHASIKCIAKSHPLYQEFRLWQFIVNLRIYRKETDVDVTQELLPTEADYVTLFEWLNEKKEIDQKAFFKYPPFGFKKTTSNYRWNYVEDKPYPCNETHAQIIARLGKAHIPKAFLSKEKEETLWHILYSIEDKQEIEKALHSFANKNNLSEEFIEQFKNFPPFKKEYGSYSAKAIKKLLPLMRMGKYWSIENIDNGTRIRINKIIDGEYDENIRERVRQKAINLTDITHFRALPLWLACYLVYDRHSEVKDIVKWKTPKDIDLYLKSFKQHSLRNPIVEQVITETLRTVRDIWQQVGHIDEIHIELGREMKNPADKRARMSQQMIKNENTNLRIKALLTEFLNPEFGIENVRPYSPSQQDLLRIYEEGVLNSILELPEDIGIILGKFNQTDTLKRPTRSEILRYKLWLEQKYRSPYTGEMIPLSKLFTPAYEIEHIIPQSRYFDDSLSNKVICESEINKLKDRSLGYEFIKNHHGEKVELAFDKPVEVLSVEAYEKLVHESYSHNRSKMKKLLMEDIPDQFIERQLNDSRYISKVVKSLLSNIVREENEQEAISKNVIPCTGGITDRLKKDWGINDVWNKIVLPRFIRLNELTESTRFTSINTNNTMIPSMPLELQKGFNKKRIDHRHHAMDAIIIACANRNIVNYLNNVSASKNTKITRRDLQTLLCHKDKTDNNGNYKWVIDKPWETFTQDTLTALQKITVSFKQNLRVINKTTNHYQHYENGKKIVSNQSKGDSWAIRKSMHKETVHGEVNLRMIKTVSFNEALKKPQAIVEMDLKKKILAMLELGYDTKRIKNYFEENKDTWQDINPSKIKVYYFTKETKDRYFAVRKPIDTSFDKKKIKESITDTGIQQIMLRHLETKDNDPTLAFSPDGIDEMNRNILILNKGKKHQPIYKVRVYEKAEKFTVGQKGNKRTKFVEAAKGTNLFFAIYETEEIDKDTKKVIRKRSYSTIPLNVVIERQKQGLSSAPEDENGNLPKYILSPNDLVYVPTQEEINKGEVVMPIDRDRIYKMVDSSGITANFIPASTANLIFALPKATAEIYCNGENCIQNEYGIGSPQSKNQKAITGEMVKEICFPIKVDRLGNIIQVGSCILTN

SEQ ID NO: 53 - protein, 1323 aa, Fructobacillus fructosus
MVYDVGLDIGTGSVGWVALDENGKLARAKGKNLVGVRLFDTAQTAADRRGFRTTRRRLSRRKWRLRLLDELFSAEINEIDSSFFQRLKYSYVHPKDEENKAHYYGGYLFPTEEETKKFHRSYPTIYHLRQELMAQPNKRFDIREIYLAIHHLVKYRGHFLSSQEKITIGSTYNPEDLANAIEVYADEKGLSWELNNPEQLTEIISGEAGYGLNKSMKADEALKLFEFDNNQDKVAIKTLLAGLTGNQIDFAKLFGKDISDKDEAKLWKLKLDDEALEEKSQTILSQLTDEEIELFHAVVQAYDGFVLIGLLNGADSVSAAMVQLYDQHREDRKLLKSLAQKAGLKHKRFSEIYEQLALATDEATIKNGISTARELVEESNLSKEVKEDTLRRLDENEFLPKQRTKANSVIPHQLHLAELQKILQNQGQYYPFLLDTFEKEDGQDNKIEELLRFRIPYYVGPLVTKKDVEHAGGDADNHWVERNEGFEKSRVTPWNFDKVFNRDKAARDFIERLTGNDTYLIGEKTLPQNSLRYQLFTVLNELNNVRVNGKKFDSKTKADLINDLFKARKTVSLSALKDYLKAQGKGDVTITGLADESKFNSSLSSYNDLKKTFDAEYLENEDNQETLEKIIEIQTVFEDSKIASRELSKLPLDDDQVKKLSQTHYTGWGRLSEKLLDSKIIDERGQKVSILDKLKSTSQNFMSIINNDKYGVQAWITEQNTGSSKLTFDEKVNELTTSPANKRGIKQSFAVLNDIKKAMKEEPRRVYLEFAREDQTSVRSVPRYNQLKEKYQSKSLSEEAKVLKKTLDGNKNKMSDDRYFLYFQQQGKDMYTGRPINFERLSQDYDIDHIIPQAFTKDDSLDNRVLVSRPENARKSDSFAYTDEVQKQDGSLWTSLLKSGFINRKKYERLTKAGKYLDGQKTGFIARQLVETRQIIKNVASLIEGEYENSKAVAIRSEITADMRLLVGIKKHREINSFHHAFDALLITAAGQYMQNRYPDRDSTNVYNEFDRYTNDYLKNLRQLSSRDEVRRLKSFGFVVGTMRKGNEDWSEENTSYLRKVMMFKNILTTKKTEKDRGPLNKETIFSPKSGKKLIPLNSKRSDTALYGGYSNVYSAYMTLVRANGKNLLIKIPISIANQIEVGNLKINDYIVNNPAIKKFEKILISKLPLGQLVNEDGNLIYLASNEYRHNAKQLWLSTTDADKIASISENSSDEELLEAYDILTSENVKNRFPFFKKDIDKLSQVRDEFLDSDKRIAVIQTILRGLQIDAAYQAPVKIISKKVSDWHKLQQSGGIKLSDNSEMIYQSATGIFETRVKISDLL

SEQ ID NO: 54 - protein, 1088 aa, Bacillus smithii
MNYKMGLDIGIASVGWAVINLDLKRIEDLGVRIFDKAEHPQNGESLALPRRIARSARRRLRRRKHRLERIRRLLVSENVLTKEEMNLLFKQKKQIDVWQLRVDALERKLNNDELARVLLHLAKRRGFKSNRKSERNSKESSEFLKNIEENQSILAQYRSVGEMIVKDSKFAYHKRNKLDSYSNMIARDDLEREIKLIFEKQREFNNPVCTERLEEKYLNIWSSQRPFASKEDIEKKVGFCTFEPKEKRAPKATYTFQSFIVWEHINKLRLVSPDETRALTEIERNLLYKQAFSKNKMTYYDIRKLLNLSDDIHFKGLLYDPKSSLKQIENIRFLELDSYHKIRKCIENVYGKDGIRMFNETDIDTFGYALTIFKDDEDIVAYLQNEYITKNGKRVSNLANKVYDKSLIDELLNLSFSKFAHLSMKAIRNILPYMEQGEIYSKACELAGYNFTGPKKKEKALLLPVIPNIANPVVMRALTQSRKVVNAIIKKYGSPVSIHIELARDLSHSFDERKKIQKDQTENRKKNETAIKQLIEYELTKNPTGLDIVKFKLWSEQQGRCMYSLKPIELERLLEPGYVEVDHILPYSRSLDDSYANKVLVLTKENREKGNHTPVEYLGLGSERWKKFEKFVLANKQFSKKKKQNLLRLRYEETEEKEFKERNLNDTRYISKFFANFIKEHLKFADGDGGQKVYTINGKITAHLRSRWDFNKNREESDLHHAVDAVIVACATQGMIKKITEFYKAREQNKESAKKKEPIFPQPWPHFADELKARLSKFPQESIEAFALGNYDRKKLESLRPVFVSRMPKRSVTGAAHQETLRRCVGIDEQSGKIQTAVKTKLSDIKLDKDGHFPMYQKESDPRTYEAIRQRLLEHNNDPKKAFQEPLYKPKKNGEPGPVIRTVKIIDTKNKVVHLDGSKTVAYNSNIVRTDVFEKDGKYYCVPVYTMDIMKGTLPNKAIEANKPYSEWKEMTEEYTFQFSLFPNDLVRIVLPREKTIKTSTNEEIIIKDIFAYYKTIDSATGGLELISHDRNFSLRGVGSKTLKRFEKYQVDVLGNIHKVKGEKRVGLAAPTNQKKGKTVDSLQSVSD

SEQ ID NO: 55 - protein, 1233 aa, Mycoplasma canis
MEKKRKVTLGFDLGIASVGWAIVDSETNQVYKLGSRLFDAPDTNLERRTQRGTRRLLRRRKYRNQKFYNLVKRTEVFGLSSREAIENRFRELSIKYPNIIELKTKALSQEVCPDEIAWILHDYLKNRGYFYDEKETKEDFDQQTVESMPSYKLNEFYKKYGYFKGALSQPTESEMKDNKDLKEAFFFDFSNKEWLKEINYFFNVQKNILSETFIEEFKKIFSFTRDISKGPGSDNMPSPYGIFGEFGDNGQGGRYEHIWDKNIGKCSIFTNEQRAPKYLPSALIFNFLNELANIRLYSTDKKNIQPLWKLSSVDKLNILLNLFNLPISEKKKKLTSTNINDIVKKESIKSIMISVEDIDMIKDEWAGKEPNVYGVGLSGLNIEESAKENKFKFQDLKILNVLINLLDNVGIKFEFKDRNDIIKNLELLDNLYLFLIYQKESNNKDSSIDLFIAKNESLNIENLKLKLKEFLLGAGNEFENHNSKTHSLSKKAIDEILPKLLDNNEGWNLEAIKNYDEEIKSQIEDNSSLMAKQDKKYLNDNFLKDAILPPNVKVTFQQAILIFNKIIQKFSKDFEIDKVVIELAREMTQDQENDALKGIAKAQKSKKSLVEERLEANNIDKSVFNDKYEKLIYKIFLWISQDFKDPYTGAQISVNEIVNNKVEIDHIIPYSLCFDDSSANKVLVHKQSNQEKSNSLPYEYIKQGHSGWNWDEFTKYVKRVFVNNVDSILSKKERLKKSENLLTASYDGYDKLGFLARNLNDTRYATILFRDQLNNYAEHHLIDNKKMFKVIAMNGAVTSFIRKNMSYDNKLRLKDRSDFSHHAYDAAIIALFSNKTKTLYNLIDPSLNGIISKRSEGYWVIEDRYTGEIKELKKEDWTSIKNNVQARKIAKEIEEYLIDLDDEVFFSRKTKRKTNRQLYNETIYGIATKTDEDGITNYYKKEKFSILDDKDIYLRLLREREKFVINQSNPEVIDQIIEIIESYGKENNIPSRDEAINIKYTKNKINYNLYLKQYMRSLTKSLDQFSEEFINQMIANKTFVLYNPTKNTTRKIKFLRLVNDVKINDIRKNQVINKFNGKNNEPKAFYENINSLGAIVFKNSANNFKTLSINTQIAIFGDKNWDIEDFKTYNMEKIEKYKEIYGIDKTYNFHSFIFPGTILLDKQNKEFYYISSIQTVRDIIEIKFLNKIEFKDENKNQDTSKTPKRLMFGIKSIMNNYEQVDISPFGINKKIFE

SEQ ID NO: 56 - protein, 1498 aa, Odoribacter laneus
METTLGIDLGTNSIGLALVDQEEHQILYSGVRIFPEGINKDTIGLGEKEESRNATRRAKRQMRRQYFRKKLRKAKLLELLIAYDMCPLKPEDVRRWKNWDKQQKSTVRQFPDTPAFREWLKQNPYELRKQAVTEDVTRPELGRILYQMIQRRGFLSSRKGKEEGKIFTGKDRMVGIDETRKNLQKQTLGAYLYDIAPKNGEKYRFRTERVRARYTLRDMYIREFEIIWQRQAGHLGLAHEQATRKKNIFLEGSATNVRNSKLITHLQAKYGRGHVLIEDTRITVTFQLPLKEVLGGKIEIEEEQLKFKSNESVLFWQRPLRSQKSLLSKCVFEGRNFYDPVHQKWIIAGPTPAPLSHPEFEEFRAYQFINNIIYGKNEHLTAIQREAVFELMCTESKDFNFEKIPKHLKLFEKFNFDDTTKVPACTTISQLRKLFPHPVWEEKREEIWHCFYFYDDNTLLFEKLQKDYALQTNDLEKIKKIRLSESYGNVSLKAIRRINPYLKKGYAYSTAVLLGGIRNSFGKRFEYFKEYEPEIEKAVCRILKEKNAEGEVIRKIKDYLVHNRFGFAKNDRAFQKLYHHSQAITTQAQKERLPETGNLRNPIVQQGLNELRRTVNKLLATCREKYGPSFKFDHIHVEMGRELRSSKTEREKQSRQIRENEKKNEAAKVKLAEYGLKAYRDNIQKYLLYKEIEEKGGTVCCPYTGKTLNISHTLGSDNSVQIEHIIPYSISLDDSLANKTLCDATFNREKGELTPYDFYQKDPSPEKWGASSWEEIEDRAFRLLPYAKAQRFIRRKPQESNEFISRQLNDTRYISKKAVEYLSAICSDVKAFPGQLTAELRHLWGLNNILQSAPDITFPLPVSATENHREYYVITNEQNEVIRLFPKQGETPRTEKGELLLTGEVERKVFRCKGMQEFQTDVSDGKYWRRIKLSSSVTWSPLFAPKPISADGQIVLKGRIEKGVFVCNQLKQKLKTGLPDGSYWISLPVISQTFKEGESVNNSKLTSQQVQLFGRVREGIFRCHNYQCPASGADGNFWCTLDTDTAQPAFTPIKNAPPGVGGGQIILTGDVDDKGIFHADDDLHYELPASLPKGKYYGIFTVESCDPTLIPIELSAPKTSKGENLIEGNIWVDEHTGEVRFDPKKNREDQRHHAIDAIVIALSSQSLFQRLSTYNARRENKKRGLDSTEHFPSPWPGFAQDVRQSVVPLLVSYKQNPKTLCKISKTLYKDGKKIHSCGNAVRGQLHKETVYGQRTAPGATEKSYHIRKDIRELKTSKHIGKVVDITIRQMLLKHLQENYHIDITQEFNIPSNAFFKEGVYRIFLPNKHGEPVPIKKIRMKEELGNAERLKDNINQYVNPRNNHHVMIYQDADGNLKEEIVSFWSVIERQNQGQPIYQLPREGRNIVSILQINDTFLIGLKEEEPEVYRNDLSTLSKHLYRVQKLSGMYYTFRHHLASTLNNEREEFRIQSLEAWKRANPVKVQIDEIGRITFLNGPLC

SEQ ID NO: 57 - protein, 1101 aa, Akkermansia muciniphila
MSRSLTFSFDIGYASIGWAVIASASHDDADPSVCGCGTVLFPKDDCQAFKRREYRRLRRNIRSRRVRIERIGRLLVQAQIITPEMKETSGHPAPFYLASEALKGHRTLAPIELWHVLRWYAHNRGYDNNASWSNSLSEDGGNGEDTERVKHAQDLMDKHGTATMAETICRELKLEEGKADAPMEVSTPAYKNLNTAFPRLIVEKEVRRILELSAPLIPGLTAEIIELIAQHHPLTTEQRGVLLQHGIKLARRYRGSLLFGQLIPRFDNRIISRCPVTWAQVYEAELKKGNSEQSARERAEKLSKVPTANCPEFYEYRMARILCNIRADGEPLSAEIRRELMNQARQEGKLTKASLEKAISSRLGKETETNVSNYFTLHPDSEEALYLNPAVEVLQRSGIGQILSPSVYRIAANRLRRGKSVTPNYLLNLLKSRGESGEALEKKIEKESKKKEADYADTPLKPKYATGRAPYARTVLKKVVEEILDGEDPTRPARGEAHPDGELKAHDGCLYCLLDTDSSVNQHQKERRLDTMTNNHLVRHRMLILDRLLKDLIQDFADGQKDRISRVCVEVGKELTTFSAMDSKKIQRELTLRQKSHTDAVNRLKRKLPGKALSANLIRKCRIAMDMNWTCPFTGATYGDHELENLELEHIVPHSFRQSNALSSLVLTWPGVNRMKGQRTGYDFVEQEQENPVPDKPNLHICSLNNYRELVEKLDDKKGHEDDRRRKKKRKALLMVRGLSHKHQSQNHEAMKEIGMTEGMMTQSSHLMKLACKSIKTSLPDAHIDMIPGAVTAEVRKAWDVFGVFKELCPEAADPDSGKILKENLRSLTHLHHALDACVLGLIPYIIPAHHNGLLRRVLAMRRIPEKLIPQVRPVANQRHYVLNDDGRMMLRDLSASLKENIREQLMEQRVIQHVPADMGGALLKETMQRVLSVDGSGEDAMVSLSKKKDGKKEKNQVKASKLVGVFPEGPSKLKALKAAIEIDGNYGVALDPKPVVIRHIKVFKRIMALKEQNGGKPVRILKKGMLIHLTSSKDPKHAGVWRIESIQDSKGGVKLDLQRAHCAVPKNKTHECNWREVDLISLLKKYQMKRYPTSYTGTPR

SEQ ID NO: 58 - protein, 1079 aa, Dinoroseobacter shibae
MRLGLDIGTSSIGWWLYETDGAGSDARITGVVDGGVRIFSDGRDPKSGASLAVDRRAARAMRRRRDRYLRRRATLMKVLAETGLMPADPAEAKALEALDPFALRAAGLDEPLPLPHLGRALFHLNQRRGFKSNRKTDRGDNESGKIKDATARLDMEMMANGARTYGEFLHKRRQKATDPRHVPSVRTRLSIANRGGPDGKEEAGYDFYPDRRHLEEEFHKLWAAQGAHHPELTETLRDLLFEKIFFQRPLKEPEVGLCLFSGHHGVPPKDPRLPKAHPLTQRRVLYETVNQLRVTADGREARPLTREERDQVIHALDNKKPTKSLSSMVLKLPALAKVLKLRDGERFTLETGVRDAIACDPLRASPAHPDRFGPRWSILDADAQWEVISRIRRVQSDAEHAALVDWLTEAHGLDRAHAEATAHAPLPDGYGRLGLTATTRILYQLTADVVTYADAVKACGWHHSDGRTGECFDRLPYYGEVLERHVIPGSYHPDDDDITRFGRITNPTVHIGLNQLRRLVNRIIETHGKPHQIVVELARDLKKSEEQKRADIKRIRDTTEAAKKRSEKLEELEIEDNGRNRMLLRLWEDLNPDDAMRRFCPYTGTRISAAMIFDGSCDVDHILPYSRTLDDSFPNRTLCLREANRQKRNQTPWQAWGDTPHWHAIAANLKNLPENKRWRFAPDAMTRFEGENGFLDRALKDTQYLARISRSYLDTLFTKGGHVWVVPGRFTEMLRRHWGLNSLLSDAGRGAVKAKNRTDHRHHAIDAAVIAATDPGLLNRISRAAGQGEAAGQSAELIARDTPPPWEGFRDDLRVRLDRIIVSHRADHGRIDHAARKQGRDSTAGQLHQETAYSIVDDIHVASRTDLLSLKPAQLLDEPGRSGQVRDPQLRKALRVATGGKTGKDFENALRYFASKPGPYQAIRRVRIIKPLQAQARVPVPAQDPIKAYQGGSNHLFEIWRLPDGEIEAQVITSFEAHTLEGEKRPHPAAKRLLRVHKGDMVALERDGRRVVGHVQKMDIANGLFIVPHNEANADTRNNDKSDPFKWIQIGARPAIASGIRRVSVDEIGRLRDGGTRPI

SEQ ID NO: 59 - protein, 1059 aa, Wolinella succinogenes
MIERILGVDLGISSLGWAIVEYDKDDEAANRIIDCGVRLFTAAETPKKKESPNKARREARGIRRVLNRRRVRMNMIKKLFLRAGLIQDVDLDGEGGMFYSKANRADVWELRHDGLYRLLKGDELARVLIHIAKHRGYKFIGDDEADEESGKVKKAGVVLRQNFEAAGCRTVGEWLWRERGANGKKRNKHGDYEISIHRDLLVEEVEAIFVAQQEMRSTIATDALKAAYREIAFFVRPMQRIEKMVGHCTYFPEERRAPKSAPTAEKFIAISKFFSTVIIDNEGWEQKIIERKTLEELLDFAVSREKVEFRHLRKFLDLSDNEIFKGLHYKGKPKTAKKREATLFDPNEPTELEFDKVEAEKKAWISLRGAAKLREALGNEFYGRFVALGKHADEATKILTYYKDEGQKRRELTKLPLEAEMVERLVKIGFSDFLKLSLKAIRDILPAMESGARYDEAVLMLGVPHKEKSAILPPLNKTDIDILNPTVIRAFAQFRKVANALVRKYGAFDRVHFELAREINTKGEIEDIKESQRKNEKERKEAADWIAETSFQVPLTRKNILKKRLYIQQDGRCAYTGDVIELERLFDEGYCEIDHILPRSRSADDSFANKVLCLARANQQKTDRTPYEWFGHDAARWNAFETRTSAPSNRVRTGKGKIDRLLKKNFDENSEMAFKDRNLNDTRYMARAIKTYCEQYWVFKNSHTKAPVQVRSGKLTSVLRYQWGLESKDRESHTHHAVDAIIIAFSTQGMVQKLSEYYRFKETHREKERPKLAVPLANFRDAVEEATRIENTETVKEGVEVKRLLISRPPRARVTGQAHEQTAKPYPRIKQVKNKKKWRLAPIDEEKFESFKADRVASANQKNFYETSTIPRVDVYHKKGKFHLVPIYLHEMVLNELPNLSLGTNPEAMDENFFKFSIFKDDLISIQTQGTPKKPAKIIMGYFKNMHGANMVLSSINNSPCEGFTCTPVSMDKKHKDKCKLCPEENRIAGRCLQGFLDYWSQEGLRPPRKEFECDQGVKFALDVKKYQIDPLGYYYEVKQEKRLGTIPQMRSAKKLVKK

SEQ ID NO: 60 - protein, 1428 aa, Parasutterella excrementihominis
MGKTHIIGVGLDLGGTYTGTFITSHPSDEAEHRDHSSAFTVVNSEKLSFSSKSRTAVRHRVRSYKGFDLRRRLLLLVAEYQLLQKKQTLAPEERENLRIALSGYLKRRGYARTEAETDTSVLESLDPSVFSSAPSFTNFFNDSEPLNIQWEAIANSPETTKALNKELSGQKEADFKKYIKTSFPEYSAKEILANYVEGRRAILDASKYIANLQSLGHKHRSKYLSDILQDMKRDSRITRLSEAFGSTDNLWRIIGNISNLQERAVRWYFNDAKFEQGQEQLDAVKLKNVLVRALKYLRSDDKEWSASQKQIIQSLEQSGDVLDVLAGLDPDRTIPPYEDQNNRRPPEDQTLYLNPKALSSEYGEKWKSWANKFAGAYPLLTEDLTEILKNTDRKSRIKIRSDVLPDSDYRLAYILQRAFDRSIALDECSIRRTAEDFENGVVIKNEKLEDVLSGHQLEEFLEFANRYYQETAKAKNGLWFPENALLERADLHPPMKNKILNVIVGQALGVSPAEGTDFIEEIWNSKVKGRSTVRSICNAIENERKTYGPYFSEDYKFVKTALKEGKTEKELSKKFAAVIKVLKMVSEVVPFIGKELRLSDEAQSKFDNLYSLAQLYNLIETERNGFSKVSLAAHLENAWRMTMTDGSAQCCRLPADCVRPFDGFIRKAIDRNSWEVAKRIAEEVKKSVDFTNGTVKIPVAIEANSFNFTASLTDLKYIQLKEQKLKKKLEDIQRNEENQEKRWLSKEERIRADSHGICAYTGRPLDDVGEIDHIIPRSLTLKKSESIYNSEVNLIFVSAQGNQEKKNNIYLLSNLAKNYLAAVFGTSDLSQITNEIESTVLQLKAAGRLGYFDLLSEKERACARHALFLNSDSEARRAVIDVLGSRRKASVNGTQAWFVRSIFSKVRQALAAWTQETGNELIFDAISVPAADSSEMRKRFAEYRPEFRKPKVQPVASHSIDAMCIYLAACSDPFKTKRMGSQLAIYEPINFDNLFTGSCQVIQNTPRNFSDKTNIANSPIFKETIYAERFLDIIVSRGEIFIGYPSNMPFEEKPNRISIGGKDPFSILSVLGAYLDKAPSSEKEKLTIYRVVKNKAFELFSKVAGSKFTAEEDKAAKILEALHFVTVKQDVAATVSDLIKSKKELSKDSIENLAKQKGCLKKVEYSSKEFKFKGSLIIPAAVEWGKVLWNVFKENTAEELKDENALRKALEAAWPSSFGTRNLHSKAKRVFSLPVVATQSGAVRIRRKTAFGDFVYQSQDTNNLYSSFPVKNGKLDWSSPIIHPALQNRNLTAYGYRFVDHDRSISMSEFREVYNKDDLMRIELAQGTSSRRYLRVEMPGEKFLAWFGENSISLGSSFKFSVSEVFDNKIYTENAEFTKFLPKPREDNKHNGTIFFELVGPRVIFNYIVGGAASSLKEIFSEAGKERS

SEQ ID NO: 61 - protein, 1421 aa, Streptococcus sanguinis
MTKFNKNYSIGLDIGVSSVGYAVVTEDYRVPAFKFKVLGNTEKEKIKKNLIGSTTFVSAQPAKGTRVFRVNRRRIDRRNHRITYLRDIFQKEIEKVDKNFYRRLDESFRVLGDKSEDLQIKQPFFGDKELETAYHKKYPTIYHLRKHLADADKNSPVADIREVYMAISHILKYRGHFLTLDKINPNNINMQNSWIDFIESCQEVFDLEISDESKNIADIFKSSENRQEKVKKILPYFQQELLKKDKSIFKQLLQLLFGLKTKFKDCFELEEEPDLNFSKENYDENLENFLGSLEEDFSDVFAKLKVLRDTILLSGMLTYTGATHARFSATMVERYEEHRKDLQRFKFFIKQNLSEQDYLDIFGRKTQNGFDVDKETKGYVGYITNKMVLTNPQKQKTIQQNFYDYISGKITGIEGAEYFLNKISDGTFLRKLRTSDNGAIPNQIHAYELEKIIERQGKDYPFLLENKDKLLSILTFKIPYYVGPLAKGSNSRFAWIKRATSSDILDDNDEDTRNGKIRPWNYQKLINMDETRDAFITNLIGNDIILLNEKVLPKRSLIYEEVMLQNELTRVKYKDKYGKAHFFDSELRQNIINGLFKNNSKRVNAKSLIKYLSDNHKDLNAIEIVSGVEKGKSFNSTLKTYNDLKTIFSEELLDSEIYQKELEEIIKVITVFDDKKSIKNYLTKFFGHLEILDEEKINQLSKLRYSGWGRYSAKLLLDIRDEDTGFNLLQFLRNDEENRNLTKLISDNTLSFEPKIKDIQSKSTIEDDIFDEIKKLAGSPAIKRGILNSIKIVDELVQIIGYPPHNIVIEMARENMTTEEGQKKAKTRKTKLESALKNIENSLLENGKVPHSDEQLQSEKLYLYYLQNGKDMYTLDKTGSPAPLYLDQLDQYEVDHIIPYSFLPIDSIDNKVLTHRENNQQKLNNIPDKETVANMKPFWEKLYNAKLISQTKYQRLTTSERTPDGVLTESMKAGFIERQLVETRQIIKHVARILDNRFSDTKIITLKSQLITNFRNTFHIAKIRELNDYHHAHDAYLAVVVGQTLLKVYPKLAPELIYGHHAHFNRHEENKATLRKHLYSNIMRFFNNPDSKVSKDIWDCNRDLPIIKDVIYNSQINFVKRTMIKKGAFYNQNPVGKFNKQLAANNRYPLKTKALCLDTSIYGGYGPMNSALSIIIIAERFNEKKGKIETVKEFHDIFIIDYEKFNNNPFQFLNDTSENGFLKKNNINRVLGFYRIPKYSLMQKIDGTRMLFESKSNLHKATQFKLTKTQNELFFHMKRLLTKSNLMDLKSKSAIKESQNFILKHKEEFDNISNQLSAFSQKMLGNTTSLKNLIKGYNERKIKEIDIRDETIKYFYDNFIKMFSFVKSGAPKDINDFFDNKCTVARMRPKPDKKLLNATLIHQSITGLYETRIDLSKLGED

SEQ ID NO: 62 - protein, 1181 aa, Actinomyces sp.
MLHCIAVIRVPPSEEPGFFETHADSCALCHHGCMTYAANDKAIRYRVGIDVGLRSIGFCAVEVDDEDHPIRILNSVVHVHDAGTGGPGETESLRKRSGVAARARRRGRAEKQRLKKLDVLLEELGWGVSSNELLDSHAPWHIRKRLVSEYIEDETERRQCLSVAMAHIARHRGWRNSFSKVDTLLLEQAPSDRMQGLKERVEDRTGLQFSEEVTQGELVATLLEHDGDVTIRGFVRKGGKATKVHGVLEGKYMQSDLVAELRQICRTQRVSETTFEKLVLSIFHSKEPAPSAARQRERVGLDELQLALDPAAKQPRAERAHPAFQKFKVVATLANMRIREQSAGERSLTSEELNRVARYLLNHTESESPTWDDVARKLEVPRHRLRGSSRASLETGGGLTYPPVDDTTVRVMSAEVDWLADWWDCANDESRGHMIDAISNGCGSEPDDVEDEEVNELISSATAEDMLKLELLAKKLPSGRVAYSLKTLREVTAAILETGDDLSQAITRLYGVDPGWVPTPAPIEAPVGNPSVDRVLKQVARWLKFASKRWGVPQTVNIEHTREGLKSASLLEEERERWERFEARREIRQKEMYKRLGISGPFRRSDQVRYEILDLQDCACLYCGNEINFQTFEVDHIIPRVDASSDSRRTNLAAVCHSCNSAKGGLAFGQWVKRGDCPSGVSLENAIKRVRSWSKDRLGLTEKAMGKRKSEVISRLKTEMPYEEFDGRSMESVAWMAIELKKRIEGYFNSDRPEGCAAVQVNAYSGRLTACARRAAHVDKRVRLIRLKGDDGHHKNRFDRRNHAMDALVIALMTPAIARTIAVREDRREAQQLTRAFESWKNFLGSEERMQDRWESWIGDVEYACDRLNELIDADKIPVTENLRLRNSGKLHADQPESLKKARRGSKRPRPQRYVLGDALPADVINRVTDPGLWTALVRAPGFDSQLGLPADLNRGLKLRGKRISADFPIDYFPTDSPALAVQGGYVGLEFHHARLYRIIGPKEKVKYALLRVCAIDLCGIDCDDLFEVELKPSSISMRTADAKLKEAMGNGSAKQIGWLVLGDEIQIDPTKFPKQSIGKFLKECGPVSSWRVSALDTPSKITLKPRLLSNEPLLKTSRVGGHESDLVVAECVEKIMKKTGWVVEINALCQSGLIRVIRRNALGEVRTSPKSGLPISLNLR

SEQ ID NO: 63 - protein, 1059 aa, Rhodovulum sp.
MGIRFAFDLGTNSIGWAVWRTGPGVFGEDTAASLDGSGVLIFKDGRNPKDGQSLATMRRVPRQSRKRRDRFVLRRRDLLAALRKAGLFPVDVEEGRRLAATDPYHLRAKALDESLTPHEMGRVIFHLNQRRGFRSNRKADRQDREKGKIAEGSKRLAETLAATNCRTLGEFLWSRHRGTPRTRSPTRIRMEGEGAKALYAFYPTREMVRAEFERLWTAQSRFAPDLLTPERHEEIAGILFRQRDLAPPKIGCCTFEPSERRLPRALPSVEARGIYERLAHLRITTGPVSDRGLTRPERDVLASALLAGKSLTFKAVRKTLKILPHALVNFEEAGEKGLDGALTAKLLSKPDHYGAAWHGLSFAEKDTFVGKLLDEADEERLIRRLVTENRLSEDAARRCASIPLADGYGRLGRTANTEILAALVEETDETGTVVTYAEAVRRAGERTGRNWHHSDERDGVILDRLPYYGEILQRHVVPGSGEPEEKNEAARWGRLANPTVHIGLNQLRKVVNRLIAAHGRPDQIVVELARELKLNREQKERLDRENRKNREENERRTAILAEHGQRDTAENKIRLRLFEEQARANAGIALCPYTGRAIGIAELFTSEVEIDHILPVSLTLDDSLANRVLCRREANREKRRQTPFQAFGATPAWNDIVARAAKLPPNKRWRFDPAALERFEREGGFLGRQLNETKYLSRLAKIYLGKICDPDRVYVTPGTLTGLLRARWGLNSILSDSNFKNRSDHRHHAVDAVVIGVLTRGMIQRIAHDAARAEDQDLDRVFRDVPVPFEDFRDHVRERVSTITVAVKPEHGKGGALHEDTSYGLVPDTDPNAALGNLVVRKPIRSLTAGEVDRVRDRALRARLGALAAPFRDESGRVRDAKGLAQALEAFGAENGIRRVRILKPDASVVTIADRRTGVPYRAVAPGENHHVDIVQMRDGSWRGFAASVFEVNRPGWRPEWEVKKLGGKLVMRLHKGDMVELSDKDGQRRVKVVQQIEISANRVRLSPHNDGGKLQDRHADADDPFRWDLATIPLLKDRGCVAVRVDPIGVVTLRRSNV

SEQ ID NO: 64 - protein, 1420 aa, Bifidobacterium bifidum
MSRKNYVDDYAISLDIGNASVGWSAFTPNYRLVRAKGHELIGVRLFDPADTAESRRMARTTRRRYSRRRWRLRLLDALFDQALSEIDPSFLARRKYSWVHPDDENNADCWYGSVLFDSNEQDKRFYEKYPTIYHLRKALMEDDSQHDIREIYLAIHHMVKYRGNFLVEGTLESSNAFKEDELLKLLGRITRYEMSEGEQNSDIEQDDENKLVAPANGQLADALCATRGSRSMRVDNALEALSAVNDLSREQRAIVKAIFAGLEGNKLDLAKIFVSKEFSSENKKILGIYFNKSDYEEKCVQIVDSGLLDDEEREFLDRMQGQYNAIALKQLLGRSTSVSDSKCASYDAHRANWNLIKLQLRTKENEKDINENYGILVGWKIDSGQRKSVRGESAYENMRKKANVFFKKMIETSDLSETDKNRLIHDIEEDKLFPIQRDSDNGVIPHQLHQNELKQIIKKQGKYYPFLLDAFEKDGKQINKIEGLLTFRVPYFVGPLVVPEDLQKSDNSENHWMVRKKKGEITPWNFDEMVDKDASGRKFIERLVGTDSYLLGEPTLPKNSLLYQEYEVLNELNNVRLSVRTGNHWNDKRRMRLGREEKTLLCQRLFMKGQTVTKRTAENLLRKEYGRTYELSGLSDESKFTSSLSTYGKMCRIFGEKYVNEHRDLMEKIVELQTVFEDKETLLHQLRQLEGISEADCALLVNTHYTGWGRLSRKLLTTKAGECKISDDFAPRKHSIIEIMRAEDRNLMEIITDKQLGFSDWIEQENLGAENGSSLMEVVDDLRVSPKVKRGIIQSIRLIDDISKAVGKRPSRIFLELADDIQPSGRTISRKSRLQDLYRNANLGKEFKGIADELNACSDKDLQDDRLFLYYTQLGKDMYTGEELDLDRLSSAYDIDHIIPQAVTQNDSIDNRVLVARAENARKTDSFTYMPQIADRMRNFWQILLDNGLISRVKFERLTRQNEFSEREKERFVQRSLVETRQIMKNVATLMRQRYGNSAAVIGLNAELTKEMHRYLGFSHKNRDINDYHHAQDALCVGIAGQFAANRGFFADGEVSDGAQNSYNQYLRDYLRGYREKLSAEDRKQGRAFGFIVGSMRSQDEQKRVNPRTGEVVWSEEDKDYLRKVMNYRKMLVTQKVGDDFGALYDETRYAATDPKGIKGIPFDGAKQDTSLYGGFSSAKPAYAVLIESKGKTRLVNVTMQEYSLLGDRPSDDELRKVLAKKKSEYAKANILLRHVPKMQLIRYGGGLMVIKSAGELNNAQQLWLPYEEYCYFDDLSQGKGSLEKDDLKKLLDSILGSVQCLYPWHRFTEEELADLHVAFDKLPEDEKKNVITGIVSALHADAKTANLSIVGMTGSWRRMNNKSGYTFSDEDEFIFQSPSGLFEKRVTVGELKRKAKKEVNSKYRTNEKRLPTLSGASQP

SEQ ID NO: 65 - protein, 1153 aa, Barnesiella intestinihominis
MKNILGLDLGLSSIGWSVIRENSEEQELVAMGSRVVSLTAAELSSFTQGNGVSINSQRTQKRTQRKGYDRYQLRRTLLRNKLDTLGMLPDDSLSYLPKLQLWGLRAKAVTQRIELNELGRVLLHLNQKRGYKSIKSDFSGDKKITDYVKTVKTRYDELKEMRLTIGELFFRRLTENAFFRCKEQVYPRQAYVEEFDCIMNCQRKFYPDILTDETIRCIRDEIIYYQRPLKSCKYLVSRCEFEKRFYLNAAGKKTEAGPKVSPRTSPLFQVCRLWESINNIVVKDRRNEIVFISAEQRAALFDFLNTHEKLKGSDLLKLLGLSKTYGYRLGEQFKTGIQGNKTRVEIERALGNYPDKKRLLQFNLQEESSSMVNTETGEIIPMISLSFEQEPLYRLWHVLYSIDDREQLQSVLRQKFGIDDDEVLERLSAIDLVKAGFGNKSSKAIRRILPFLQLGMNYAEACEAAGYNHSNNYTKAENEARALLDRLPAIKKNELRQPVVEKILNQMVNVVNALMEKYGRFDEIRVELARELKQSKEERSNTYKSINKNQRENEQIAKRIVEYGVPTRSRIQKYKMWEESKHCCIYCGQPVDVGDFLRGFDVEVEHIIPKSLYFDDSFANKVCSCRSCNKEKNNRTAYDYMKSKGEKALSDYVERVNTMYTNNQISKTKWQNLLTPVDKISIDFIDRQLRESQYIARKAKEILTSICYNVTATSGSVTSFLRHVWGWDTVLHDLNFDRYKKVGLTEVIEVNHRGSVIRREQIKDWSKRFDHRHHAIDALTIACTKQAYIQRLNNLRAEEGPDFNKMSLERYIQSQPHFSVAQVREAVDRILVSFRAGKRAVTPGKRYIRKNRKRISVQSVLIPRGALSEESVYGVIHVWEKDEQGHVIQKQRAVMKYPITSINREMLDKEKVVDKRIHRILSGRLAQYNDNPKEAFAKPVYIDKECRIPIRTVRCFAKPAINTLVPLKKDDKGNPVAWVNPGNNHHVAIYRDEDGKYKERTVTFWEAVDRCRVGIPAIVTQPDTIWDNILQRNDISENVLESLPDVKWQFVLSLQQNEMFILGMNEEDYRYAMDQQDYALLNKYLYRVQKLSKSDYSFRYHTETSVEDKYDGKPNLKLSMQMGKLKRVSIKSLLGLNPHKVHISVLGEIKEIS

SEQ ID NO: 66 - protein, 1052 aa, Aminomonas paucivorans
MIGEHVRGGCLFDDHWTPNWGAFRLPNTVRTFTKAENPKDGSSLAEPRRQARGLRRRLRRKTQRLEDLRRLLAKEGVLSLSDLETLFRETPAKDPYQLRAEGLDRPLSFPEWVRVLYHITKHRGFQSNRRNPVEDGQERSRQEEEGKLLSGVGENERLLREGGYRTAGEMLARDPKFQDHRRNRAGDYSHTLSRSLLLEEARRLFQSQRTLGNPHASSNLEEAFLHLVAFQNPFASGEDIRNKAGHCSLEPDQIRAPRRSASAETFMLLQKTGNLRLIHRRTGEERPLTDKEREQIHLLAWKQEKVTHKTLRRHLEIPEEWLFTGLPYHRSGDKAEEKLFVHLAGIHEIRKALDKGPDPAVWDTLRSRRDLLDSIADTLTFYKNEDEILPRLESLGLSPENARALAPLSFSGTAHLSLSALGKLLPHLEEGKSYTQARADAGYAAPPPDRHPKLPPLEEADWRNPVVFRALTQTRKVVNALVRRYGPPWCIHLETARELSQPAKVRRRIETEQQANEKKKQQAEREFLDIVGTAPGPGDLLKMRLWREQGGFCPYCEEYLNPTRLAEPGYAEMDHILPYSRSLDNGWHNRVLVHGKDNRDKGNRTPFEAFGGDTARWDRLVAWVQASHLSAPKKRNLLREDFGEEAERELKDRNLTDTRFITKTAATLLRDRLTFHPEAPKDPVMTLNGRLTAFLRKQWGLHKNRKNGDLHHALDAAVLAVASRSFVYRLSSHNAAWGELPRGREAENGFSLPYPAFRSEVLARLCPTREEILLRLDQGGVGYDEAFRNGLRPVFVSRAPSRRLRGKAHMETLRSPKWKDHPEGPRTASRIPLKDLNLEKLERMVGKDRDRKLYEALRERLAAFGGNGKKAFVAPFRKPCRSGEGPLVRSLRIFDSGYSGVELRDGGEVYAVADHESMVRVDVYAKKNRFYLVPVYVADVARGIVKNRAIVAHKSEEEWDLVDGSFDFRFSLFPGDLVEIEKKDGAYLGYYKSCHRGDGRLLLDRHDRMPRESDCGTFYVSTRKDVLSMSKYQVDPLGEIRLVGSEKPPFVL

SEQ ID NO: 67 - protein, 1062 aa, Ralstonia syzygii
MAEKQHRWGLDIGTNSIGWAVIALIEGRPAGLVATGSRIFSDGRNPKDGSSLAVERRGPRQMRRRRDRYLRRRDRFMQALINVGLMPGDAAARKALVTENPYVLRQRGLDQALTLPEFGRALFHLNQRRGFQSNRKTDRATAKESGKVKNAIAAFRAGMGNARTVGEALARRLEDGRPVRARMVGQGKDEHYELYIAREWIAQEFDALWASQQRFHAEVLADAARDRLRAILLFQRKLLPVPVGKCFLEPNQPRVAAALPSAQRFRLMQELNHLRVMTLADKRERPLSFQERNDLLAQLVARPKCGFDMLRKIVFGANKEAYRFTIESERRKELKGCDTAAKLAKVNALGTRWQALSLDEQDRLVCLLLDGENDAVLADALREHYGLTDAQIDTLLGLSFEDGHMRLGRSALLRVLDALESGRDEQGLPLSYDKAVVAAGYPAHTADLENGERDALPYYGELLWRYTQDAPTAKNDAERKFGKIANPTVHIGLNQLRKLVNALIQRYGKPAQIVVELARNLKAGLEEKERIKKQQTANLERNERIRQKLQDAGVPDNRENRLRMRLFEELGQGNGLGTPCIYSGRQISLQRLFSNDVQVDHILPFSKTLDDSFANKVLAQHDANRYKGNRGPFEAFGANRDGYAWDDIRARAAVLPRNKRNRFAETAMQDWLHNETDFLARQLTDTAYLSRVARQYLTAICSKDDVYVSPGRLTAMLRAKWGLNRVLDGVMEEQGRPAVKNRDDHRHHAIDAVVIGATDRAMLQQVATLAARAREQDAERLIGDMPTPWPNFLEDVRAAVARCVVSHKPDHGPEGGLHNDTAYGIVAGPFEDGRYRVRHRVSLFDLKPGDLSNVRCDAPLQAELEPIFEQDDARAREVALTALAERYRQRKVWLEELMSVLPIRPRGEDGKTLPDSAPYKAYKGDSNYCYELFINERGRWDGELISTFRANQAAYRRFRNDPARFRRYTAGGRPLLMRLCINDYIAVGTAAERTIFRVVKMSENKITLAEHFEGGTLKQRDADKDDPFKYLTKSPGALRDLGARRIFVDLIGRVLDPGIKGD

SEQ ID NO: 68 - protein, 1329 aa, Catenibacterium mitsuokai
IVDYCIGLDLGTGSVGWAVVDMNHRLMKRNGKHLWGSRLFSNAETAANRRASRSIRRRYNKRRERIRLLRAILQDMVLEKDPTFFIRLEHTSFLDEEDKAKYLGTDYKDNYNLFIDEDFNDYTYYHKYPTIYHLRKALCESTEKADPRLIYLALHHIVKYRGNFLYEGQKFNMDASNIEDKLSDIFTQFTSFNNIPYEDDEKKNLEILEILKKPLSKKAKVDEVMTLIAPEKDYKSAFKELVTGIAGNKMNVTKMILCEPIKQGDSEIKLKFSDSNYDDQFSEVEKDLGEYVEFVDALHNVYSWVELQTIMGATHTDNASISEAMVSRYNKHHDDLKLLKDCIKNNVPNKYFDMFRNDSEKSKGYYNYINRPSKAPVDEFYKYVKKCIEKVDTPEAKQILNDIELENFLLKQNSRTNGSVPYQMQLDEMIKIIDNQAEYYPILKEKREQLLSILTFRIPYYFGPLNETSEHAWIKRLEGKENQRILPWNYQDIVDVDATAEGFIKRMRSYCTYFPDEEVLPKNSLIVSKYEVYNELNKIRVDDKLLEVDVKNDIYNELFMKNKTVTEKKLKNWLVNNQCCSKDAEIKGFQKENQFSTSLTPWIDFTNIFGKIDQSNFDLIENIIYDLTVFEDKKIMKRRLKKKYALPDDKVKQILKLKYKDWSRLSKKLLDGIVADNRFGSSVTVLDVLEMSRLNLMEIINDKDLGYAQMIEEATSCPEDGKFTYEEVERLAGSPALKRGIWQSLQIVEEITKVMKCRPKYIYIEFERSEEAKERTESKIKKLENVYKDLDEQTKKEYKSVLEELKGFDNTKKISSDSLFLYFTQLGKCMYSGKKLDIDSLDKYQIDHIVPQSLVKDDSFDNRVLVVPSENQRKLDDLVVPFDIRDKMYRFWKLLFDHELISPKKFYSLIKTEYTERDEERFINRQLVETRQITKNVTQIIEDHYSTTKVAAIRANLSHEFRVKNHIYKNRDINDYHHAHDAYIVALIGGFMRDRYPNMHDSKAVYSEYMKMFRKNKNDQKRWKDGFVINSMNYPYEVDGKLIWNPDLINEIKKCFYYKDCYCTTKLDQKSGQLFNLTVLSNDAHADKGVTKAVVPVNKNRSDVHKYGGFSGLQYTIVAIEGQKKKGKKTELVKKISGVPLHLKAASINEKINYIEEKEGLSDVRIIKDNIPVNQMIEMDGGEYLLTSPTEYVNARQLVLNEKQCALIADIYNAIYKQDYDNLDDILMIQLYIELTNKMKVLYPAYRGIAEKFESMNENYVVISKEEKANIIKQMLIVMHRGPQNGNIVYDDFKISDRIGRLKTKNHNLNNIVFISQSPTGIYTKKYKL

SEQ ID NO: 69 - protein, 1314 aa, Mycoplasma synoviae
MLRLYCANNLVLNNVQNLWKYLLLLIFDKKIIFLFKIKVILIRRYMENNNKEKIVIGFDLGVASVGWSIVNAETKEVIDLGVRLFSEPEKADYRRAKRTTRRLLRRKKFKREKFHKLILKNAEIFGLQSRNEILNVYKDQSSKYRNILKLKINALKEEIKPSELVWILRDYLQNRGYFYKNEKLTDEFVSNSFPSKKLHEHYEKYGFFRGSVKLDNKLDNKKDKAKEKDEEEESDAKKESEELIFSNKQWINEIVKVFENQSYLTESFKEEYLKLFNYVRPFNKGPGSKNSRTAYGVFSTDIDPETNKFKDYSNIWDKTIGKCSLFEEEIRAPKNLPSALIFNLQNEICTIKNEFTEFKNWWLNAEQKSEILKFVFTELFNWKDKKYSDKKFNKNLQDKIKKYLLNFALENFNLNEEILKNRDLENDTVLGLKGVKYYEKSNATADAALEFSSLKPLYVFIKFLKEKKLDLNYLLGLENTEILYFLDSIYLAISYSSDLKERNEWFKKLLKELYPKIKNNNLEIIENVEDIFEITDQEKFESFSKTHSLSREAFNHIIPLLLSNNEGKNYESLKHSNEELKKRTEKAELKAQQNQKYLKDNFLKEALVPLSVKTSVLQAIKIFNQIIKNFGKKYEISQVVIEMARELTKPNLEKLLNNATNSNIKILKEKLDQTEKFDDFTKKKFIDKIENSVVFRNKLFLWFEQDRKDPYTQLDIKINEIEDETEIDHVIPYSKSADDSWFNKLLVKKSTNQLKKNKTVWEYYQNESDPEAKWNKFVAWAKRIYLVQKSDKESKDNSEKNSIFKNKKPNLKFKNITKKLFDPYKDLGFLARNLNDTRYATKVFRDQLNNYSKHHSKDDENKLFKVVCMNGSITSFLRKSMWRKNEEQVYRFNFWKKDRDQFFHHAVDASIIAIFSLLTKTLYNKLRVYESYDVQRREDGVYLINKETGEVKKADKDYWKDQHNFLKIRENAIEIKNVLNNVDFQNQVRYSRKANTKLNTQLFNETLYGVKEFENNFYKLEKVNLFSRKDLRKFILEDLNEESEKNKKNENGSRKRILTEKYIVDEILQILENEEFKDSKSDINALNKYMDSLPSKFSEFFSQDFINKCKKENSLILTFDAIKHNDPKKVIKIKNLKFFREDATLKNKQAVHKDSKNQIKSFYESYKCVGFIWLKNKNDLEESIFVPINSRVIHFGDKDKDIFDFDSYNKEKLLNEINLKRPENKKFNSINEIEFVKFVKPGALLLNFENQQIYYISTLESSSLRAKIKLLNKMDKGKAVSMKKITNPDEYKIIEHVNPLGINLNWTKKLENNN

SEQ ID NO: 70 - protein, 1473 aa, Flavobacterium branchiophilum
MAKILGLDLGTNSIGWAVVERENIDFSLIDKGVRIFSEGVKSEKGIESSRAAERTGYRSARKIKYRRKLRKYETLKVLSLNRMCPLSIEEVEEWKKSGFKDYPLNPEFLKWLSTDEESNVNPYFFRDRASKHKVSLFELGRAFYHIAQRRGFLSNRLDQSAEGILEEHCPKIEAIVEDLISIDEISTNITDYFFETGILDSNEKNGYAKDLDEGDKKLVSLYKSLLAILKKNESDFENCKSEIIERLNKKDVLGKVKGKIKDISQAMLDGNYKTLGQYFYSLYSKEKIRNQYTSREEHYLSEFITICKVQGIDQINEEEKINEKKFDGLAKDLYKAIFFQRPLKSQKGLIGKCSFEKSKSRCAISHPDFEEYRMWTYLNTIKIGTQSDKKLRFLTQDEKLKLVPKFYRKNDFNFDVLAKELIEKGSSFGFYKSSKKNDFFYWFNYKPTDTVAACQVAASLKNAIGEDWKTKSFKYQTINSNKEQVSRTVDYKDLWHLLTVATSDVYLYEFAIDKLGLDEKNAKAFSKTKLKKDFASLSLSAINKILPYLKEGLLYSHAVFVANIENIVDENIWKDEKQRDYIKTQISEIIENYTLEKSRFEIINGLLKEYKSENEDGKRVYYSKEAEQSFENDLKKKLVLFYKSNEIENKEQQETIFNELLPIFIQQLKDYEFIKIQRLDQKVLIFLKGKNETGQIFCTEEKGTAEEKEKKIKNRLKKLYHPSDIEKFKKKIIKDEFGNEKIVLGSPLTPSIKNPMAMRALHQLRKVLNALILEGQIDEKTIIHIEMARELNDANKRKGIQDYQNDNKKFREDAIKEIKKLYFEDCKKEVEPTEDDILRYQLWMEQNRSEIYEEGKNISICDIIGSNPAYDIEHTIPRSRSQDNSQMNKTLCSQRFNREVKKQSMPIELNNHLEILPRIAHWKEEADNLTREIEIISRSIKAAATKEIKDKKIRRRHYLTLKRDYLQGKYDRFIWEEPKVGFKNSQIPDTGIITKYAQAYLKSYFKKVESVKGGMVAEFRKIWGIQESFIDENGMKHYKVKDRSKHTHHTIDAITIACMTKEKYDVLAHAWTLEDQQNKKEARSIIEASKPWKTFKEDLLKIEEEILVSHYTPDNVKKQAKKIVRVRGKKQFVAEVERDVNGKAVPKKAASGKTIYKLDGEGKKLPRLQQGDTIRGSLHQDSIYGAIKNPLNTDEIKYVIRKDLESIKGSDVESIVDEVVKEKIKEAIANKVLLLSSNAQQKNKLVGTVWMNEEKRIAINKVRIYANSVKNPLHIKEHSLLSKSKHVHKQKVYGQNDENYAMAIYELDGKRDFELINIFNLAKLIKQGQGFYPLHKKKEIKGKIVFVPIEKRNKRDVVLKRGQQVVFYDKEVENPKDISEIVDFKGRIYIIEGLSIQRIVRPSGKVDEYGVIMLRYFKEARKADDIKQDNFKPDGVFKLGENKPTRKMNHNQFTAFVEGIDFKVLPSGKFEKI

SEQ ID NO: 71 - protein, 1391 aa, Eubacterium yurii
MENKQYYIGLDVGTNSVGWAVTDTSYNLLRAKGKDMWGARLFEKANTAAERRTKRTSRRRSEREKARKAMLKELFADEINRVDPSFFIRLEESKFFLDDRSENNRQRYTLFNDATFTDKDYYEKYKTIFHLRSALINSDEKFDVRLVFLAILNLFSHRGHFLNASLKGDGDIQGMDVFYNDLVESCEYFEIELPRITNIDNFEKILSQKGKSRTKILEELSEELSISKKDKSKYNLIKLISGLEASVVELYNIEDIQDENKKIKIGFRESDYEESSLKVKEIIGDEYFDLVERAKSVHDMGLLSNIIGNSKYLCEARVEAYENHHKDLLKIKELLKKYDKKAYNDMFRKMTDKNYSAYVGSVNSNIAKERRSVDKRKIEDLYKYIEDTALKNIPDDNKDKIEILEKIKLGEFLKKQLTASNGVIPNQLQSRELRAILKKAENYLPFLKEKGEKNLTVSEMIIQLFEFQIPYYVGPLDKNPKKDNKANSWAKIKQGGRILPWNFEDKVDVKGSRKEFIEKMVRKCTYISDEHTLPKQSLLYEKFMVLNEINNIKIDGEKISVEAKQKIYNDLFVKGKKVSQKDIKKELISLNIMDKDSVLSGTDTVCNAYLSSIGKFTGVFKEEINKQSIVDMIEDIIFLKTVYGDEKRFVKEEIVEKYGDEIDKDKIKRILGFKFSNWGNLSKSFLELEGADVGTGEVRSIIQSLWETNFNLMELLSSRFTYMDELEKRVKKLEKPLSEWTIEDLDDMYLSSPVKRMIWQSMKIVDEIQTVIGYAPKRIFVEMTRSEGEKVRTKSRKDRLKELYNGIKEDSKQWVKELDSKDESYFRSKKMYLYYLQKGRCMYSGEVIELDKLMDDNLYDIDHIYPRSFVKDDSLDNLVLVKKEINNRKQNDPITPQIQASCQGFWKILHDQGFMSNEKYSRLTRKTQEFSDEEKLSFINRQIVETGQATKCMAQILQKSMGEDVDVVFSKARLVSEFRHKFELFKSRLINDFHHANDAYLNIVVGNSYFVKFTRNPANFIKDARKNPDNPVYKYHMDRFFERDVKSKSEVAWIGQSEGNSGTIVIVKKTMAKNSPLITKKVEEGHGSITKETIVGVKEIKFGRNKVEKADKTPKKPNLQAYRPIKTSDERLCNILRYGGRTSISISGYCLVEYVKKRKTIRSLEAIPVYLGRKDSLSEEKLLNYFRYNLNDGGKDSVSDIRLCLPFISTNSLVKIDGYLYYLGGKNDDRIQLYNAYQLKMKKEEVEYIRKIEKAVSMSKFDEIDREKNPVLTEEKNIELYNKIQDKFENTVFSKRMSLVKYNKKDLSFGDFLKNKKSKFEEIDLEKQCKVLYNIIFNLSNLKEVDLSDIGGSKSTGKCRCKKNITNYKEFKLIQQSITGLYSCEKDLMTI

SEQ ID NO: 72 - protein, 1131 aa, unidentified
MAQHVFGLDIGIASVGWAILGEQRIIDLGVRCFDKAETAKEGDPLNLTRRQARLLRRRLYRRAWRLTQLSRLLKRKGLIADAKLFAKAPSYGDSAWELRRQGLDRLLTPLEWARVIYHQCKHRGFHWTSKAEEAKADSDAEGGRVKQGLAHTKALMQAKNYRSAAEMVLAEFPDAQRNKRGQYDKALSRVLLGEELALLFATQRRLGNPHASDFFEKLILGDGDRKSGLFWQQKPALSGADLLKMLGKCTFEKGEYRAPKASFSVERHVWLTRLNNLRIVVDGRSRPLNEAERQAALLLPYQTETSKYKTLKNAFIKAGLWGDGVRFGGLAYPSQAQIDAEKTKDPEDQFLVKLPAWHELRKAFKAAGHEALWQQISTPALDGDPTLLDQIATVLSVYKDGAEVVQQLRQLALPEPAASIAVLEKISFDKFSSLSLKALRRIVPLMQSGLRYDEAVAQIPEYGHHSQRIEPGAAKHLYLPPFYEAQRKYAGKGDHIGSMQFRDDADIPRNPVVLRALNQARKVVNALIREYGSPIAVNIEMARDLSRPLDERNKVKRAQEEFRDRNDRARSEFERDFGYKPKAAAFEKWMLYREQLGQCAYSQQPLDIQRVLDDHNYAQVDHALPYSRSYDDSKNNKVLVLTHENQNKGNRTAFEYLTSFPDGEDGERWRTFVAWVQGNKAYRMAKRNRLLRKNYGVDESKGFIDRNLNDTRYICKFFKNYVEEHLQLAARADGDTARRCVVVNGQLTAFLRARWGLTKVRGDSDRHHALDAAVVAACTHGMVKALADYSRRKEISFLQEGFPDPETGEILNPAAFDRARQHFPEPWTHFAHELKARLFTDDLAALREDMQRLGSYTTEDLGRLRTLFVSRAPQRRSGGAVHKETIYAQPESLKQQGGVIEKILLTSLKLQDFDKLLNPESNDHFVEPHRNERLYAAIRQRLEQFGGRADKAFGPDNLFHKPDKNNQPTGPVVRSIKLVRGKQTGIPIRGGLAKNDSMLRVDIFTKAGKFHLVPVYVHHRVTGLPNRAIVAFKDEDEWTLIDESFAFLFSVYPNDYVKVTLKKEQQSGYYSGADRSTGAMNLWAHDRAASVGKDGLIRGIGVKTALSVEKFNVDVLGRIYLAPPETRSGLA

SEQ ID NO: 73 - protein, 1197 aa, Porphyromonas sp.
MLMSKHVLGLDLGVGSIGWCLIALDAQGDPAEILGMGSRVVPLNNATKAIEAFNAGAAFTASQERTARRTMRRGFARYQLRRYRLRRELEKVGMLPDAALIQLPLLELWELRERAATAGRRLTLPELGRVLCHINQKRGYRHVKSDAAAIVGDEGEKKKDSNSAYLAGIRANDEKLQAEHKTVGQYFAEQLRQNQSESPTGGISYRIKDQIFSRQCYIDEYDQIMAVQRVHYPDILTDEFIRMLRDEVIFMQRPLKSCKHLVSLCEFEKQERVMRVQQDDGKGGWQLVERRVKFGPKVAPKSSPLFQLCCIYEAVNNIRLTRPNGSPCDITPEERAKIVAHLQSSASLSFAALKKLLKEKALIADQLTSKSGLKGNSTRVALASALQPYPQYHHLLDMELETRMMTVQLTDEETGEVTEREVAVVTDSYVRKPLYRLWHILYSIEEREAMRRALITQLGMKEEDLDGGLLDQLYRLDFVKPGYGNKSAKFICKLLPQLQQGLGYSEACAAVGYRHSNSPTSEEITERTLLEKIPLLQRNELRQPLVEKILNQMINLVNALKAEYGIDEVRVELARELKMSREERERMARNNKDREERNKGVAAKIRECGLYPTKPRIQKYMLWKEAGRQCLYCGRSIEEEQCLREGGMEVEHIIPKSVLYDDSYGNKTCACRRCNKEKGNRTALEYIRAKGREAEYMKRINDLLKEKKISYSKHQRLRWLKEDIPSDFLERQLRLTQYISRQAMAILQQGIRRVSASEGGVTARLRSLWGYGKILHTLNLDRYDSMGETERVSREGEATEELHITNWSKRMDHRHHAIDALVVACTRQSYIQRLNRLSSEFGREDKKKEDQEAQEQQATETGRLSNLERWLTQRPHFSVRTVSDKVAEILISYRPGQRVVTRGRNIYRKKMADGREVSCVQRGVLVPRGELMEASFYGKILSQGRVRIVKRYPLHDLKGEVVDPHLRELITTYNQELKSREKGAPIPPLCLDKDKKQEVRSVRCYAKTLSLDKAIPMCFDEKGEPTAFVKSASNHHLALYRTPKGKLVESIVTFWDAVDRARYGIPLVITHPREVMEQVLQRGDIPEQVLSLLPPSDWVFVDSLQQDEMVVIGLSDEELQRALEAQNYRKISEHLYRVQKMSSSYYVFRYHLETSVADDKNTSGRIPKFHRVQSLKAYEERNIRKVRVDLLGRISLL

SEQ ID NO: 74 - protein, 1265 aa, Mycoplasma ovipneumoniae
MHNKKNITIGFDLGIASIGWAIIDSTTSKILDWGTRTFEERKTANERRAFRSTRRNIRRKAYRNQRFINLILKYKDLFELKNISDIQRANKKDTENYEKIISFFTEIYKKCAAKHSNILEVKVKALDSKIEKLDLIWILHDYLENRGFFYDLEEENVADKYEGIEHPSILLYDFFKKNGFFKSNSSIPKDLGGYSFSNLQWVNEIKKLFEVQEINPEFSEKFLNLFTSVRDYAKGPGSEHSASEYGIFQKDEKGKVFKKYDNIWDKTIGKCSFFVEENRSPVNYPSYEIFNLLNQLINLSTDLKTTNKKIWQLSSNDRNELLDELLKVKEKAKIISISLKKNEIKKIILKDFGFEKSDIDDQDTIEGRKIIKEEPTTKLEVTKHLLATIYSHSSDSNWININNILEFLPYLDAICIILDREKSRGQDEVLKKLTEKNIFEVLKIDREKQLDFVKSIFSNTKFNFKKIGNFSLKAIREFLPKMFEQNKNSEYLKWKDEEIRRKWEEQKSKLGKTDKKTKYLNPRIFQDEIISPGTKNTFEQAVLVLNQIIKKYSKENIIDAIIIESPREKNDKKTIEEIKKRNKKGKGKTLEKLFQILNLENKGYKLSDLETKPAKLLDRLRFYHQQDGIDLYTLDKINIDQLINGSQKYEIEHIIPYSMSYDNSQANKILTEKAENLKKGKLIASEYIKRNGDEFYNKYYEKAKELFINKYKKNKKLDSYVDLDEDSAKNRFRFLTLQDYDEFQVEFLARNLNDTRYSTKLFYHALVEHFENNEFFTYIDENSSKHKVKISTIKGHVTKYFRAKPVQKNNGPNENLNNNKPEKIEKNRENNEHHAVDAAIVAIIGNKNPQIANLLTLADNKTDKKFLLHDENYKENIETGELVKIPKFEVDKLAKVEDLKKIIQEKYEEAKKHTAIKFSRKTRTILNGGLSDETLYGFKYDEKEDKYFKIIKKKLVTSKNEELKKYFENPFGKKADGKSEYTVLMAQSHLSEFNKLKEIFEKYNGFSNKTGNAFVEYMNDLALKEPTLKAEIESAKSVEKLLYYNFKPSDQFTYHDNINNKSFKRFYKNIRIIEYKSIPIKFKILSKHDGGKSFKDTLFSLYSLVYKVYENGKESYKSIPVTSQMRNFGIDEFDFLDENLYNKEKLDIYKSDFAKPIPVNCKPVFVLKKGSILKKKSLDIDDFKETKETEEGNYYFISTISKRFNRDTAYGLKPLKLSVVKPVAEPSTNPIFKEYIPIHLDELGNEYPVKIKEHTDDEKLMCTIK

SEQ ID NO: 75 - protein, 1409 aa, Wolinella succinogenes
MLVSPISVDLGGKNTGFFSFTDSLDNSQSGTVIYDESFVLSQVGRRSKRHSKRNNLRNKLVKRLFLLILQEHHGLSIDVLPDEIRGLFNKRGYTYAGFELDEKKKDALESDTLKEFLSEKLQSIDRDSDVEDFLNQIASNAESFKDYKKGFEAVFASATHSPNKKLELKDELKSEYGENAKELLAGLRVTKEILDEFDKQENQGNLPRAKYFEELGEYIATNEKVKSFFDSNSLKLTDMTKLIGNISNYQLKELRRYFNDKEMEKGDIWIPNKLHKITERFVRSWHPKNDADRQRRAELMKDLKSKEIMELLTTTEPVMTIPPYDDMNNRGAVKCQTLRLNEEYLDKHLPNWRDIAKRLNHGKFNDDLADSTVKGYSEDSTLLHRLLDTSKEIDIYELRGKKPNELLVKTLGQSDANRLYGFAQNYYELIRQKVRAGIWVPVKNKDDSLNLEDNSNMLKRCNHNPPHKKNQIHNLVAGILGVKLDEAKFAEFEKELWSAKVGNKKLSAYCKNIEELRKTHGNTFKIDIEELRKKDPAELSKEEKAKLRLTDDVILNEWSQKIANFFDIDDKHRQRFNNLFSMAQLHTVIDTPRSGFSSTCKRCTAENRFRSETAFYNDETGEFHKKATATCQRLPADTQRPFSGKIERYIDKLGYELAKIKAKELEGMEAKEIKVPIILEQNAFEYEESLRKSKTGSNDRVINSKKDRDGKKLAKAKENAEDRLKDKDKRIKAFSSGICPYCGDTIGDDGEIDHILPRSHTLKIYGTVFNPEGNLIYVHQKCNQAKADSIYKLSDIKAGVSAQWIEEQVANIKGYKTFSVLSAEQQKAFRYALFLQNDNEAYKKVVDWLRTDQSARVNGTQKYLAKKIQEKLTKMLPNKHLSFEFILADATEVSELRRQYARQNPLLAKAEKQAPSSHAIDAVMAFVARYQKVFKDGTPPNADEVAKLAMLDSWNPASNEPLTKGLSTNQKIEKMIKSGDYGQKNMREVFGKSIFGENAIGERYKPIVVQEGGYYIGYPATVKKGYELKNCKVVTSKNDIAKLEKIIKNQDLISLKENQYIKIFSINKQTISELSNRYFNMNYKNLVERDKEIVGLLEFIVENCRYYTKKVDVKFAPKYIHETKYPFYDDWRRFDEAWRYLQENQNKTSSKDRFVIDKSSLNEYYQPDKNEYKLDVDTQPIWDDFCRWYFLDRYKTANDKKSIRIKARKTFSLLAESGVQGKVFRAKRKIPTGYAYQALPMDNNVIAGDYANILLEANSKTLSLVPKSGISIEKQLDKKLDVIKKTDVRGLAIDNNSFFNADFDTHGIRLIVENTSVKVGNFPISAIDKSAKRMIFRALFEKEKGKRKKKTTISFKESGPVQDYLKVFLKKIVKIQLRTDGSISNIVVRKNAADFTLSFRSEHIQKLLK

SEQ ID NO: 76 - protein, 1345 aa, Streptococcus mutans
MKKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 77 - protein, 1218 aa, Prevotella timonensis
MNKRILGLDTGTNSLGWAVVDWDEHAQSYELIKYGDVIFQEGVKIEKGIESSKAAERSGYKAIRKQYFRRRLRKIQVLKVLVKYHLCPYLSDDDLRQWHLQKQYPKSDELMLWQRTSDEEGKNPYYDRHRCLHEKLDLTVEADRYTLGRALYHLTQRRGFLSNRLDTSADNKEDGVVKSGISQLSTEMEEAGCEYLGDYFYKLYDAQGNKVRIRQRYTDRNKHYQHEFDAICEKQELSSELIEDLQRAIFFQLPLKSQRHGVGRCTFERGKPRCADSHPDYEEFRMLCFVNNIQVKGPHDLELRPLTYEEREKIEPLFFRKSKPNFDFEDIAKALAGKKNYAWIHDKEERAYKFNYRMTQGVPGCPTIAQLKSIFGDDWKTGIAETYTLIQKKNGSKSLQEMVDDVWNVLYSFSSVEKLKEFAHHKLQLDEESAEKFAKIKLSHSFAALSLKAIRKFLPFLRKGMYYTHASFFANIPTIVGKEIWNKEQNRKYIMENVGELVFNYQPKHREVQGTIEMLIKDFLANNFELPAGATDKLYHPSMIETYPNAQRNEFGILQLGSPRTNAIRNPMAMRSLHILRRVVNQLLKESIIDENTEVHVEYARELNDANKRRAIADRQKEQDKQHKKYGDEIRKLYKEETGKDIEPTQTDVLKFQLWEEQNHHCLYTGEQIGITDFIGSNPKFDIEHTIPQSVGGDSTQMNLTLCDNRFNREVKKAKLPTELANHEEILTRIEPWKNKYEQLVKERDKQRTFAGMDKAVKDIRIQKRHKLQMEIDYWRGKYERFTMTEVPEGFSRRQGTGIGLISRYAGLYLKSLFHQADSRNKSNVYVVKGVATAEFRKMWGLQSEYEKKCRDNHSHHCMDAITIACIGKREYDLMAEYYRMEETFKQGRGSKPKFSKPWATFTEDVLNIYKNLLVVHDTPNNMPKHTKKYVQTSIGKVLAQGDTARGSLHLDTYYGAIERDGEIRYVVRRPLSSFTKPEELENIVDETVKRTIKEAIADKNFKQAIAEPIYMNEEKGILIKKVRCFAKSVKQPINIRQHRDLSKKEYKQQYHVMNENNYLLAIYEGLVKNKVVREFEIVSYIEAAKYYKRSQDRNIFSSIVPTHSTKYGLPLKTKLLMGQLVLMFEENPDEIQVDNTKDLVKRLYKVVGIEKDGRIKFKYHQEARKEGLPIFSTPYKNNDDYAPIFRQSINNINILVDGIDFTIDILGKVTLKE

SEQ ID NO: 78 - protein, 1021 aa, Clostridium cellulolyticum
MKYTLGLDVGIASVGWAVIDKDNNKIIDLGVRCFDKAEESKTGESLATARRIARGMRRRISRRSQRLRLVKKLFVQYEIIKDSSEFNRIFDTSRDGWKDPWELRYNALSRILKPYELVQVLTHITKRRGFKSNRKEDLSTTKEGVVITSIKNNSEMLRTKNYRTIGEMIFMETPENSNKRNKVDEYIHTIAREDLLNEIKYIFSIQRKLGSPFVTEKLEHDFLNIWEFQRPFASGDSILSKVGKCTLLKEELRAPTSCYTSEYFGLLQSINNLVLVEDNNTLTLNNDQRAKIIEYAHFKNEIKYSEIRKLLDIEPEILFKAHNLTHKNPSGNNESKKFYEMKSYHKLKSTLPTDIWGKLHSNKESLDNLFYCLTVYKNDNEIKDYLQANNLDYLIEYIAKLPTFNKFKHLSLVAMKRIIPFMEKGYKYSDACNMAELDFTGSSKLEKCNKLTVEPIIENVTNPVVIRALTQARKVINAIIQKYGLPYMVNIELAREAGMTRQDRDNLKKEHENNRKAREKISDLIRQNGRVASGLDILKWRLWEDQGGRCAYSGKPIPVCDLLNDSLTQIDHIYPYSRSMDDSYMNKVLVLTDENQNKRSYTPYEVWGSTEKWEDFEARIYSMHLPQSKEKRLLNRNFITKDLDSFISRNLNDTRYISRFLKNYIESYLQFSNDSPKSCVVCVNGQCTAQLRSRWGLNKNREESDLHHALDAAVIACADRKIIKEITNYYNERENHNYKVKYPLPWHSFRQDLMETLAGVFISRAPRRKITGPAHDETIRSPKHFNKGLTSVKIPLTTVTLEKLETMVKNTKGGISDKAVYNVLKNRLIEHNNKPLKAFAEKIYKPLKNGTNGAIIRSIRVETPSYTGVFRNEGKGISDNSLMVRVDVFKKKDKYYLVPIYVAHMIKKELPSKAIVPLKPESQWELIDSTHEFLFSLYQNDYLVIKTKKGITEGYYRSCHRGTGSLSLMPHFANNKNVKIDIGVRTAISIEKYNVDILGNKSIVKGEPRRGMEKYNSFKSN

SEQ ID NO: 79 - protein, 1629 aa, Francisella tularensis
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 80 - protein, 1168 aa, Azospirillum sp.
MARPAFRAPRREHVNGWTPDPHRISKPFFILVSWHLLSRVVIDSSSGCFPGTSRDHTDKFAEWECAVQPYRLSFDLGTNSIGWGLLNLDRQGKPREIRALGSRIFSDGRDPQDKASLAVARRLARQMRRRRDRYLTRRTRLMGALVRFGLMPADPAARKRLEVAVDPYLARERATRERLEPFEIGRALFHLNQRRGYKPVRTATKPDEEAGKVKEAVERLEAAIAAAGAPTLGAWFAWRKTRGETLRARLAGKGKEAAYPFYPARRMLEAEFDTLWAEQARHHPDLLTAEAREILRHRIFHQRPLKPPPVGRCTLYPDDGRAPRALPSAQRLRLFQELASLRVIHLDLSERPLTPAERDRIVAFVQGRPPKAGRKPGKVQKSVPFEKLRGLLELPPGTGFSLESDKRPELLGDETGARIAPAFGPGWTALPLEEQDALVELLLTEAEPERAIAALTARWALDEATAAKLAGATLPDFHGRYGRRAVAELLPVLERETRGDPDGRVRPIRLDEAVKLLRGGKDHSDFSREGALLDALPYYGAVLERHVAFGTGNPADPEEKRVGRVANPTVHIALNQLRHLVNAILARHGRPEEIVIELARDLKRSAEDRRREDKRQADNQKRNEERKRLILSLGERPTPRNLLKLRLWEEQGPVENRRCPYSGETISMRMLLSEQVDIDHILPFSVSLDDSAANKVVCLREANRIKRNRSPWEAFGHDSERWAGILARAEALPKNKRWRFAPDALEKLEGEGGLRARHLNDTRHLSRLAVEYLRCVCPKVRVSPGRLTALLRRRWGIDAILAEADGPPPEVPAETLDPSPAEKNRADHRHHALDAVVIGCIDRSMVQRVQLAAASAEREAAAREDNIRRVLEGFKEEPWDGFRAELERRARTIVVSHRPEHGIGGALHKETAYGPVDPPEEGFNLVVRKPIDGLSKDEINSVRDPRLRRALIDRLAIRRRDANDPATALAKAAEDLAAQPASRGIRRVRVLKKESNPIRVEHGGNPSGPRSGGPFHKLLLAGEVHHVDVALRADGRRWVGHWVTLFEAHGGRGADGAAAPPRLGDGERFLMRLHKGDCLKLEHKGRVRVMQVVKLEPSSNSVVVVEPHQVKTDRSKHVKISCDQLRARGARRVTVDPLGRVRVHAPGARVGIGGDAGRTAMEPAEDIS

SEQ ID NO: 81 - protein, 1364 aa, unidentified
MKNLKEYYIGLDIGTASVGWAVTDESYNIPKFNGKKMWGVRLFDDAKTAEERRTQRGSRRRLNRRKERINLLQDLFATEISKVDPNFFLRLDNSDLYREDKDEKLKSKYTLFNDKDFKDRDYHKKYPTIHHLIMDLIEDEGKKDIRLLYLACHYLLKNRGHFIFEGQKFDTKNSFDKSINDLKIHLRDEYNIDLEFNNEDLIEIITDTTLNKTNKKKELKNIVGDTKFLKAISAIMIGSSQKLVDLFEDGEFEETTVKSVDFSTTAFDDKYSEYEEALGDTISLLNILKSIYDSSILENLLKDADKSKDGNKYISKAFVKKFNKHGKDLKTLKRIIKKYLPSEYANIFRNKSINDNYVAYTKSNITSNKRTKASKFTKQEDFYKFIKKHLDTIKETKLNSSENEDLKLIDEMLTDIEFKTFIPKLKSSDNGVIPYQLKLMELKKILDNQSKYYDFLNESDEYGTVKDKVESIMEFRIPYYVGPLNPDSKYAWIKRENTKITPWNFKDIVDLDSSREEFIDRLIGRCTYLKEEKVLPKASLIYNEFMVLNELNNLKLNEFLITEEMKKAIFEELFKTKKKVTLKAVSNLLKKEFNLTGDILLSGTDGDFKQGLNSYIDFKNIIGDKVDRDDYRIKIEEIIKLIVLYEDDKTYLKKKIKSAYKNDFTDDEIKKIAALNYKDWGRLSKRFLTGIEGVDKTTGEKGSIIYFMREYNLNLMELMSGHYTFTEEVEKLNPVENRELCYEMVDELYLSPSVKRMLWQSLRVVDEIKRIIGKDPKKIFIEMARAKEAKNSRKESRKNKLLEFYKFGKKAFINEIGEERYNYLLNEINSEEESKFRWDNLYLYYTQLGRCMYSLEPIDLADLKSNNIYDQDHIYPKSKIYDDSLENRVLVKKNLNHEKGNQYPIPEKVLNKNAYGFWKILFDKGLIGQKKYTRLTRRTPFEERELAEFIERQIVETRQATKETANLLKNICQDSEIVYSKAENASRFRQEFDIIKCRTVNDLHHMHDAYLNIVVGNVYNTKFTKNPLNFIKDKDNVRSYNLENMFKYDVVRGSYTAWIADDSEGNVKAATIKKVKRELEGKNYRFTRMSYIGTGGLYDQNLMRKGKGQIPQKENTNKSNIEKYGGYNKASSAYFALIESDGKAGRERTLETIPIMVYNQEKYGNTEAVDKYLKDNLELQDPKILKDKIKINSLIKLDGFLYNIKGKTGDSLSIAGSVQLIVNKEEQKLIKKMDKFLVKKKDNKDIKVTSFDNIKEEELIKLYKTLSDKLNNGIYSNKRNNQAKNISEALDKFKEISIEEKIDVLNQIILLFQSYNNGCNLKSIGLSAKTGVVFIPKKLNYKECKLINQSITGLFENEVDLLNL

SEQ ID NO: 82 - protein, 1119 aa, Lactobacillus coryniformis
MGYRIGLDVGITSTGYAVLKTDKNGLPYKILTLDSVIYPRAENPQTGASLAEPRRIKRGLRRRTRRTKFRKQRTQQLFIHSGLLSKPEIEQILATPQAKYSVYELRVAGLDRRLTNSELFRVLYFFIGHRGFKSNRKAELNPENEADKKQMGQLLNSIEEIRKAIAEKGYRTVGELYLKDPKYNDHKRNKGYIDGYLSTPNRQMLVDEIKQILDKQRELGNEKLTDEFYATYLLGDENRAGIFQAQRDFDEGPGAGPYAGDQIKKMVGKDIFEPTEDRAAKATYTFQYFNLLQKMTSLNYQNTTGDTWHTLNGLDRQAIIDAVFAKAEKPTKTYKPTDFGELRKLLKLPDDARFNLVNYGSLQTQKEIETVEKKTRFVDFKAYHDLVKVLPEEMWQSRQLLDHIGTALTLYSSDKRRRRYFAEELNLPAELIEKLLPLNFSKFGHLSIKSMQNIIPYLEMGQVYSEATTNTGYDFRKKQISKDTIREEITNPVVRRAVTKTIKIVEQIIRRYGKPDGINIELARELGRNFKERGDIQKRQDKNRQTNDKIAAELTELGIPVNGQNIIRYKLHKEQNGVDPYTGDQIPFERAFSEGYEVDHIIPYSISWDDSYTNKVLTSAKCNREKGNRIPMVYLANNEQRLNALTNIADNIIRNSRKRQKLLKQKLSDEELKDWKQRNINDTRFITRVLYNYFRQAIEFNPELEKKQRVLPLNGEVTSKIRSRWGFLKVREDGDLHHAIDATVIAAITPKFIQQVTKYSQHQEVKNNQALWHDAEIKDAEYAAEAQRMDADLFNKIFNGFPLPWPEFLDELLARISDNPVEMMKSRSWNTYTPIEIAKLKPVFVVRLANHKISGPAHLDTIRSAKLFDEKGIVLSRVSITKLKINKKGQVATGDGIYDPENSNNGDKVVYSAIRQALEAHNGSGELAFPDGYLEYVDHGTKKLVRKVRVAKKVSLPVRLKNKAAADNGSMVRIDVFNTGKKFVFVPIYIKDTVEQVLPNKAIARGKSLWYQITESDQFCFSLYPGDMVHIESKTGIKPKYSNKENNTSVVPIKNFYGYFDGADIATASILVRAHDSSYTARSIGIAGLLKFEKYQVDYFGRYHKVHEKKRQLFVKRDE

SEQ ID NO: 83 - protein, 1688 aa, Ignavibacterium album
MEFKKVLGLDIGTNSIGCALLSLPKSIQDYGKGGRLEWLTSRVIPLDADYMKAFIDGKNGLPQVITPAGKRRQKRGSRRLKHRYKLRRSRLIRVFKTLNWLPEDFPLDNPKRIKETISTEGKFSFRISDYVPISDESYREFYREFGYPENEIEQVIEEINFRRKTKGKNKNPMIKLLPEDWVVYYLRKKALIKPTTKEELIRIIYLFNQRRGFKSSRKDLTETAILDYDEFAKRLAEKEKYSAENYETKFVSITKVKEVVELKTDGRKGKKRFKVILEDSRIEPYEIERKEKPDWEGKEYTFLVTQKLEKGKFKQNKPDLPKEEDWALCTTALDNRMGSKHPGEFFFDELLKAFKEKRGYKIRQYPVNRWRYKKELEFIWTKQCQLNPELNNLNINKEILRKLATVLYPSQSKFFGPKIKEFENSDVLHIISEDIIYYQRDLKSQKSLISECRYEKRKGIDGEIYGLKCIPKSSPLYQEFRIWQDIHNIKVIRKESEVNGKKKINIDETQLYINENIKEKLFELFNSKDSLSEKDILELISLNIINSGIKISKKEEETTHRINLFANRKELKGNETKSRYRKVFKKLGFDGEYILNHPSKLNRLWHSDYSNDYADKEKTEKSILSSLGWKNRNGKWEKSKNYDVFNLPLEVAKAIANLPPLKKEYGSYSALAIRKMLVVMRDGKYWQHPDQIAKDQENTSLMLFDKNLIQLTNNQRKVLNKYLLTLAEVQKRSTLIKQKLNEIEHNPYKLELVSDQDLEKQVLKSFLEKKNESDYLKGLKTYQAGYLIYGKHSEKDVPIVNSPDELGEYIRKKLPNNSLRNPIVEQVIRETIFIVRDVWKSFGIIDEIHIELGRELKNNSEERKKTSESQEKNFQEKERARKLLKELLNSSNFEHYDENGNKIFSSFTVNPNPDSPLDIEKFRIWKNQSGLTDEELNKKLKDEKIPTEIEVKKYILWLTQKCRSPYTGKIIPLSKLFDSNVYEIEHIIPRSKMKNDSTNNLVICELGVNKAKGDRLAANFISESNGKCKFGEVEYTLLKYGDYLQYCKDTFKYQKAKYKNLLATEPPEDFIERQINDTRYIGRKLAELLTPVVKDSKNIIFTIGSITSELKITWGLNGVWKDILRPRFKRLESIINKKLIFQDEDDPNKYHFDLSINPQLDKEGLKRLDHRHHALDATIIAATTREHVRYLNSLNAADNDEEKREYFLSLCNHKIRDFKLPWENFTSEVKSKLLSCVVSYKESKPILSDPFNKYLKWEYKNGKWQKVFAIQIKNDRWKAVRRSMFKEPIGTVWIKKIKEVSLKEAIKIQAIWEEVKNDPVRKKKEKYIYDDYAQKVIAKIVQELGLSSSMRKQDDEKLNKFINEAKVSAGVNKNLNTTNKTIYNLEGRFYEKIKVAEYVLYKAKRMPLNKKEYIEKLSLQKMFNDLPNFILEKSILDNYPEILKELESDNKYIIEPHKKNNPVNRLLLEHILEYHNNPKEAFSTEGLEKLNKKAINKIGKPIKYITRLDGDINEEEIFRGAVFETDKGSNVYFVMYENNQTKDREFLKPNPSISVLKAIEHKNKIDFFAPNRLGFSRIILSPGDLVYVPTNDQYVLIKDNSSNETIINWDDNEFISNRIYQVKKFTGNSCYFLKNDIASLILSYSASNGVGEFGSQNISEYSVDDPPIRIKDVCIKIRVDRLGNVRPL

SEQ ID NO: 84 - protein, 1011 aa, unidentified
MSSKAIDSLEQLDLFKPQEYTLGLDLGIKSIGWAILSGERIANAGVYLFETAEELNSTGNKLISKAAERGRKRRIRRMLDRKARRGRHIRYLLEREGLPTDELEEVVVHQSNRTLWDVRAEAVERKLTKQELAAVLFHLVRHRGYFPNTKKLPPDDESDSADEEQGKINRATSRLREELKASDCKTIGQFLAQNRDRQRNREGDYSNLMARKLVFEEALQILAFQRKQGHELSKDFEKTYLDVLMGQRSGRSPKLGNCSLIPSELRAPSSAPSTEWFKFLQNLGNLQISNAYREEWSIDAPRRAQIIDACSQRSTSSYWQIRRDFQIPDEYRFNLVNYERRDPDVDLQEYLQQQERKTLANFRNWKQLEKIIGTGHPIQTLDEAARLITLIKDDEKLSDQLADLLPEASDKAITQLCELDFTTAAKISLEAMYRILPHMNQGMGFFDACQQESLPEIGVPPAGDRVPPFDEMYNPVVNRVLSQSRKLINAVIDEYGMPAKIRVELARDLGKGRELRERIKLDQLDKSKQNDQRAEDFRAEFQQAPRGDQSLRYRLWKEQNCTCPYSGRMIPVNSVLSEDTQIDHILPISQSFDNSLSNKVLCFTEENAQKSNRTPFEYLDAADFQRLEAISGNWPEAKRNKLLHKSFGKVAEEWKSRALNDTRYLTSALADHLRHHLPDSKIQTVNGRITGYLRKQWGLEKDRDKHTHHAVDAIVVACTTPAIVQQVTLYHQDIRRYKKLGEKRPTPWPETFRQDVLDVEEEIFITRQPKKVSGGIQTKDTLRKHRSKPDRQRVALTKVKLADLERLVEKDASNRNLYEHLKQCLEESGDQPTKAFKAPFYMPSGPEAKQRPILSKVTLLREKPEPPKQLTELSGGRRYDSMAQGRLDIYRYKPGGKRKDEYRVVLQRMIDLMRGEENVHVFQKGVPYDQGPEIEQNYTFLFSLYFDDLVEFQRSADSEVIRGYYRTFNIANGQLKISTYLEGRQDFDFFGANRLAHFAKVQVNLLGKVIK

SEQ ID NO: 85 - protein, 1156 aa, Ruminococcus albus
MGNYYLGLDVGIGSIGWAVINIEKKRIEDFNVRIFKSGEIQEKNRNSRASQQCRRSRGLRRLYRRKSHRKLRLKNYLSIIGLTTSEKIDYYYETADNNVIQLRNKGLSEKLTPEEIAACLIHICNNRGYKDFYEVNVEDIEDPDERNEYKEEHDSIVLISNLMNEGGYCTPAEMICNCREFDEPNSVYRKFHNSAASKNHYLITRHMLVKEVDLILENQSKYYGILDDKTIAKIKDIIFAQRDFEIGPGKNERFRRFTGYLDSIGKCQFFKDQERGSRFTVIADIYAFVNVLSQYTYTNNRGESVFDTSFANDLINSALKNGSMDKRELKAIAKSYHIDISDKNSDTSLTKCFKYIKVVKPLFEKYGYDWDKLIENYTDTDNNVLNRIGIVLSQAQTPKRRREKLKALNIGLDDGLINELTKLKLSGTANVSYKYMQGSIEAFCEGDLYGKYQAKFNKEIPDIDENAKPQKLPPFKNEDDCEFFKNPVVFRSINETRKLINAIIDKYGYPAAVNIETADELNKTFEDRAIDTKRNNDNQKENDRIVKEIIECIKCDEVHARHLIEKYKLWEAQEGKCLYSGETITKEDMLRDKDKLFEVDHIVPYSLILDNTINNKALVYAEENQKKGQRTPLMYMNEAQAADYRVRVNTMFKSKKCSKKKYQYLMLPDLNDQELLGGWRSRNLNDTRYICKYLVNYLRKNLRFDRSYESSDEDDLKIRDHYRVFPVKSRFTSMFRRWWLNEKTWGRYDKAELKKLTYLDHAADAIIIANCRPEYVVLAGEKLKLNKMYHQAGKRITPEYEQSKKACIDNLYKLFRMDRRTAEKLLSGHGRLTPIIPNLSEEVDKRLWDKNIYEQFWKDDKDKKSCEELYRENVASLYKGDPKFASSLSMPVISLKPDHKYRGTITGEEAIRVKEIDGKLIKLKRKSISEITAESINSIYTDDKILIDSLKTIFEQADYKDVGDYLKKTNQHFFTTSSGKRVNKVTVIEKVPSRWLRKEIDDNNFSLLNDSSYYCIELYKDSKGDNNLQGIAMSDIVHDRKTKKLYLKPDFNYPDDYYTHVMYIFPGDYLRIKSTSKKSGEQLKFEGYFISVKNVNENSFRFISDNKPCAKDKRVSITKKDIVIKLAVDLMGKVQGENNGKGISCGEPLSLLKEKN

SEQ ID NO: 86 - protein, 1356 aa, Lactobacillus farciminis
MTKKEQPYNIGLDIGTSSVGWAVTNDNYDLLNIKKKNLWGVRLFEEAQTAKETRLNRSTRRRYRRRKNRINWLNEIFSEELAKTDPSFLIRLQNSWVSKKDPDRKRDKYNLFIDGPYTDKEYYREFPTIFHLRKELILNKDKADIRLIYLALHNILKYRGNFTYEHQKFNISNLNNNLSKELIELNQQLIKYDISFPDDCDWNHISDILIGRGNATQKSSNILKDFTLDKETKKLLKEVINLILGNVAHLNTIFKTSLTKDEEKLNFSGKDIESKLDDLDSILDDDQFTVLDAANRIYSTITLNEILNGESYFSMAKVNQYENHAIDLCKLRDMWHTTKNEEAVEQSRQAYDDYINKPKYGTKELYTSLKKFLKVALPTNLAKEAEEKISKGTYLVKPRNSENGVVPYQLNKIEMEKIIDNQSQYYPFLKENKEKLLSILSFRIPYYVGPLQSAEKNPFAWMERKSNGHARPWNFDEIVDREKSSNKFIRRMTVTDSYLVGEPVLPKNSLIYQRYEVLNELNNIRITENLKTNPIGSRLTVETKQRIYNELFKKYKKVTVKKLTKWLIAQGYYKNPILIGLSQKDEFNSTLTTYLDMKKIFGSSFMEDNKNYDQIEELIEWLTIFEDKQILNEKLHSSKYSYTPDQIKKISNMRYKGWGRLSKKILMDITTETNTPQLLQLSNYSILDLMWATNNNFISIMSNDKYDFKNYIENHNLNKNEDQNISDLVNDIHVSPALKRGITQSIKIVQEIVKFMGHAPKHIFIEVTRETKKSEITTSREKRIKRLQSKLLNKANDFKPQLREYLVPNKKIQEELKKHKNDLSSERIMLYFLQNGKSLYSEESLNINKLSDYQVDHILPRTYIPDDSLENKALVLAKENQRKADDLLLNSNVIDRNLERWTYMLNNNMIGLKKFKNLTRRVITDKDKLGFIHRQLVQTSQMVKGVANILDNMYKNQGTTCIQARANLSTAFRKALSGQDDTYHFKHPELVKNRNVNDFHHAQDAYLASFLGTYRLRRFPTNEMLLMNGEYNKFYGQVKELYSKKKKLPDSRKNGFIISPLVNGTTQYDRNTGEIIWNVGFRDKILKIFNYHQCNVTRKTEIKTGQFYDQTIYSPKNPKYKKLIAQKKDMDPNIYGGFSGDNKSSITIVKIDNNKIKPVAIPIRLINDLKDKKTLQNWLEENVKHKKSIQIIKNNVPIGQIIYSKKVGLLSLNSDREVANRQQLILPPEHSALLRLLQIPDEDLDQILAFYDKNILVEILQELITKMKKFYPFYKGEREFLIANIENFNQATTSEKVNSLEELITLLHANSTSAHLIFNNIEKKAFGRKTHGLTLNNTDFIYQSVTGLYETRIHIE

SEQ ID NO: 87 - protein, 1096 aa, Eubacterium dolichum
MMEVFMGRLVLGLDIGITSVGFGIIDLDESEIVDYGVRLFKEGTAAENETRRTKRGGRRLKRRRVTRREDMLHLLKQAGIISTSFHPLNNPYDVRVKGLNERLNGEELATALLHLCKHRGSSVETIEDDEAKAKEAGETKKVLSMNDQLLKSGKYVCEIQKERLRTNGHIRGHENNFKTRAYVDEAFQILSHQDLSNELKSAIITIISRKRMYYDGPGGPLSPTPYGRYTYFGQKEPIDLIEKMRGKCSLFPNEPRAPKLAYSAELFNLLNDLNNLSIEGEKLTSEQKAMILKIVHEKGKITPKQLAKEVGVSLEQIRGFRIDTKGSPLLSELTGYKMIREVLEKSNDEHLEDHVFYDEIAEILTKTKDIEGRKKQISELSSDLNEESVHQLAGLTKFTAYHSLSFKALRLINEEMLKTELNQMQSITLFGLKQNNELSVKGMKNIQADDTAILSPVAKRAQRETFKVVNRLREIYGEFDSIVVEMAREKNSEEQRKAIRERQKFFEMRNKQVADIIGDDRKINAKLREKLVLYQEQDGKTAYSLEPIDLKLLIDDPNAYEVDHIIPISISLDDSITNKVLVTHRENQEKGNLTPISAFVKGRFTKGSLAQYKAYCLKLKEKNIKTNKGYRKKVEQYLLNENDIYKYDIQKEFINRNLVDTSYASRVVLNTLTTYFKQNEIPTKVFTVKGSLTNAFRRKINLKKDRDEDYGHHAIDALIIASMPKMRLLSTIFSRYKIEDIYDESTGEVFSSGDDSMYYDDRYFAFIASLKAIKVRKFSHKIDTKPNRSVADETIYSTRVIDGKEKVVKKYKDIYDPKFTALAEDILNNAYQEKYLMALHDPQTFDQIVKVVNYYFEEMSKSEKYFTKDKKGRIKISGMNPLSLYRDEHGMLKKYSKKGDGPAITQMKYFDGVLGNHIDISAHYQVRDKKVVLQQISPYRTDFYYSKENGYKFVTIRYKDVRWSEKKKKYVIDQQDYAMKKAEKKIDDTYEFQFSMHRDELIGITKAEGEALIYPDETWHNFNFFFHAGETPEILKFTATNNDKSNKIEVKPIHCYCKMRLMPTISKKIVRIDKYATDVVGNLYKVKKNTLKFEFD

SEQ ID NO: 88 - protein, 1132 aa, Nitratifractor salsuginis
MKKILGVDLGITSFGYAILQETGKDLYRCLDNSVVMRNNPYDEKSGESSQSIRSTQKSMRRLIEKRKKRIRCVAQTMERYGILDYSETMKINDPKNNPIKNRWQLRAVDAWKRPLSPQELFAIFAHMAKHRGYKSIATEDLIYELELELGLNDPEKESEKKADERRQVYNALRHLEELRKKYGGETIAQTIHRAVEAGDLRSYRNHDDYEKMIRREDIEEEIEKVLLRQAELGALGLPEEQVSELIDELKACITDQEMPTIDESLFGKCTFYKDELAAPAYSYLYDLYRLYKKLADLNIDGYEVTQEDREKVIEWVEKKIAQGKNLKKITHKDLRKILGLAPEQKIFGVEDERIVKGKKEPRTFVPFFFLADIAKFKELFASIQKHPDALQIFRELAEILQRSKTPQEALDRLRALMAGKGIDTDDRELLELFKNKRSGTRELSHRYILEALPLFLEGYDEKEVQRILGFDDREDYSRYPKSLRHLHLREGNLFEKEENPINNHAVKSLASWALGLIADLSWRYGPFDEIILETTRDALPEKIRKEIDKAMREREKALDKIIGKYKKEFPSIDKRLARKIQLWERQKGLDLYSGKVINLSQLLDGSADIEHIVPQSLGGLSTDYNTIVTLKSVNAAKGNRLPGDWLAGNPDYRERIGMLSEKGLIDWKKRKNLLAQSLDEIYTENTHSKGIRATSYLEALVAQVLKRYYPFPDPELRKNGIGVRMIPGKVTSKTRSLLGIKSKSRETNFHHAEDALILSTLTRGWQNRLHRMLRDNYGKSEAELKELWKKYMPHIEGLTLADYIDEAFRRFMSKGEESLFYRDMFDTIRSISYWVDKKPLSASSHKETVYSSRHEVPTLRKNILEAFDSLNVIKDRHKLTTEEFMKRYDKEIRQKLWLHRIGNTNDESYRAVEERATQIAQILTRYQLMDAQNDKEIDEKFQQALKELITSPIEVTGKLLRKMRFVYDKLNAMQIDRGLVETDKNMLGIHISKGPNEKLIFRRMDVNNAHELQKERSGILCYLNEMLFIFNKKGLIHYGCLRSYLEKGQGSKYIALFNPRFPANPKAQPSKFTSDSKIKQVGIGSATGIIKAHLDLDGHVRSYEVFGTLPEGSIEWFKEESGYGRVEDDPHH

SEQ ID NO: 89 - protein, 1173 aa, Rhodospirillum rubrum
MRPIEPWILGLDIGTDSLGWAVFSCEEKGPPTAKELLGGGVRLFDSGRDAKDHTSRQAERGAFRRARRQTRTWPWRRDRLIALFQAAGLTPPAAETRQIALALRREAVSRPLAPDALWAALLHLAHHRGFRSNRIDKRERAAAKALAKAKPAKATAKATAPAKEADDEAGFWEGAEAALRQRMAASGAPTVGALLADDLDRGQPVRMRYNQSDRDGVVAPTRALIAEELAEIVARQSSAYPGLDWPAVTRLVLDQRPLRSKGAGPCAFLPGEDRALRALPTVQDFIIRQTLANLRLPSTSADEPRPLTDEEHAKALALLSTARFVEWPALRRALGLKRGVKFTAETERNGAKQAARGTAGNLTEAILAPLIPGWSGWDLDRKDRVFSDLWAARQDRSALLALIGDPRGPTRVTEDETAEAVADAIQIVLPTGRASLSAKAARAIAQAMAPGIGYDEAVTLALGLHHSHRPRQERLARLPYYAAALPDVGLDGDPVGPPPAEDDGAAAEAYYGRIGNISVHIALNETRKIVNALLHRHGPILRLVMVETTRELKAGADERKRMIAEQAERERENAEIDVELRKSDRWMANARERRQRVRLARRQNNLCPYTSTPIGHADLLGDAYDIDHVIPLARGGRDSLDNMVLCQSDANKTKGDKTPWEAFHDKPGWIAQRDDFLARLDPQTAKALAWRFADDAGERVARKSAEDEDQGFLPRQLTDTGYIARVALRYLSLVTNEPNAVVATNGRLTGLLRLAWDITPGPAPRDLLPTPRDALRDDTAARRFLDGLTPPPLAKAVEGAVQARLAALGRSRVADAGLADALGLTLASLGGGGKNRADHRHHFIDAAMIAVTTRGLINQINQASGAGRILDLRKWPRTNFEPPYPTFRAEVMKQWDHIHPSIRPAHRDGGSLHAATVFGVRNRPDARVLVQRKPVEKLFLDANAKPLPADKIAEIIDGFASPRMAKRFKALLARYQAAHPEVPPALAALAVARDPAFGPRGMTANTVIAGRSDGDGEDAGLITPFRANPKAAVRTMGNAVYEVWEIQVKGRPRWTHRVLTRFDRTQPAPPPPPENARLVMRLRRGDLVYWPLESGDRLFLVKKMAVDGRLALWPARLATGKATALYAQLSCPNINLNGDQGYCVQSAEGIRKEKIRTTSCTALGRLRLSKKAT

SEQ ID NO: 90 - protein, 1348 aa, Finegoldia magna
MKSEKKYYIGLDVGTNSVGWAVTDEFYNILRAKGKDLWGVRLFEKADTAANTRIFRSGRRRNDRKGMRLQILREIFEDEIKKVDKDFYDRLDESKFWAEDKKVSGKYSLFNDKNFSDKQYFEKFPTIFHLRKYLMEEHGKVDIRYYFLAINQMMKRRGHFLIDGQISHVTDDKPLKEQLILLINDLLKIELEEELMDSIFEILADVNEKRTDKKNNLKELIKGQDFNKQEGNILNSIFESIVTGKAKIKNIISDEDILEKIKEDNKEDFVLTGDSYEENLQYFEEVLQENITLFNTLKSTYDFLILQSILKGKSTLSDAQVERYDEHKKDLEILKKVIKKYDEDGKLFKQVFKEDNGNGYVSYIGYYLNKNKKITAKKKISNIEFTKYVKGILEKQCDCEDEDVKYLLGKIEQENFLLKQISSINSVIPHQIHLFELDKILENLAKNYPSFNNKKEEFTKIEKIRKTFTFRIPYYVGPLNDYHKNNGGNAWIFRNKGEKIRPWNFEKIVDLHKSEEEFIKRMLNQCTYLPEETVLPKSSILYSEYMVLNELNNLRINGKPLDTDVKLKLIEELFKKKTKVTLKSIRDYMVRNNFADKEDFDNSEKNLEIASNMKSYIDFNNILEDKFDVEMVEDLIEKITIHTGNKKLLKKYIEETYPDLSSSQIQKIINLKYKDWGRLSRKLLDGIKGTKKETEKTDTVINFLRNSSDNLMQIIGSQNYSFNEYIDKLRKKYIPQEISYEVVENLYVSPSVKKMIWQVIRVTEEITKVMGYDPDKIFIEMAKSEEEKKTTISRKNKLLDLYKAIKKDERDSQYEKLLTGLNKLDDSDLRSRKLYLYYTQMGRDMYTGEKIDLDKLFDSTHYDKDHIIPQSMKKDDSIINNLVLVNKNANQTTKGNIYPVPSSIRNNPKIYNYWKYLMEKEFISKEKYNRLIRNTPLTNEELGGFINRQLVETRQSTKAIKELFEKFYQKSKIIPVKASLASDLRKDMNTLKSREVNDLHHAHDAFLNIVAGDVWNREFTSNPINYVKENREGDKVKYSLSKDFTRPRKSKGKVIWTPEKGRKLIVDTLNKPSVLISNESHVKKGELFNATIAGKKDYKKGKIYLPLKKDDRLQDVSKYGGYKAINGAFFFLVEHTKSKKRIRSIELFPLHLLSKFYEDKNTVLDYAINVLQLQDPKIIIDKINYRTEIIIDNFSYLISTKSNDGSITVKPNEQMYWRVDEISNLKKIENKYKKDAILTEEDRKIMESYIDKIYQQFKAGKYKNRRTTDTIIEKYEIIDLDTLDNKQLYQLLVAFISLSYKTSNNAVDFTVIGLGTECGKPRITNLPDNTYLVYKSITGIYEKRIRIK

SEQ ID NO: 91 - protein, 1114 aa, Eubacterium rectale
MNYTEKEKLFMKYILALDIGIASVGWAILDKESETVIEAGSNIFPEASAADNQLRRDMRGAKRNNRRLKTRINDFIKLWENNNLSIPQFKSTEIVGLKVRAITEEITLDELYLILYSYLKHRGISYLEDALDDTVSGSSAYANGLKLNAKELETHYPCEIQQERLNTIGKYRGQSQIINENGEVLDLSNVFTIGAYRKEIQRVFEIQKKYHPELTDEFCDGYMLIFNRKRKYYEGPGNEKSRTDYGRFTTKLDANGNYITEDNIFEKLIGKCSVYPDELRAAAASYTAQEYNVLNDLNNLTINGRKLEENEKHEIVERIKSSNTINMRKIISDCMGENIDDFAGARIDKSGKEIFHKFEVYNKMRKALLEIGIDISNYSREELDEIGYIMTINTDKEAMMEAFQKSWIDLSDDVKQCLINMRKTNGALFNKWQSFSLKIMNELIPEMYAQPKEQMTLLTEMGVTKGTQEEFAGLKYIPVDVVSEDIFNPVVRRSVRISFKILNAVLKKYKALDTIVIEMPRDRNSEEQKKRINDSQKLNEKEMEYIEKKLAVTYGIKLSPSDFSSQKQLSLKLKLWNEQDGICLYSGKTIDPNDIINNPQLFEIDHIIPRSISFDDARSNKVLVYRSENQKKGNQTPYYYLTHSHSEWSFEQYKATVMNLSKKKEYAISRKKIQNLLYSEDITKMDVLKGFINRNINDTSYASRLVLNTIQNFFMANEADTKVKVIKGSYTHQMRCNLKLDKNRDESYSHHAVDAMLIGYSELGYEAYHKLQGEFIDFETGEILRKDMWDENMSDEVYADYLYGKKWANIRNEVVKAEKNVKYWHYVMRKSNRGLCNQTIRGTREYDGKQYKINKLDIRTKEGIKVFAKLAFSKKDSDRERLLVYLNDRRTFDDLCKIYEDYSDAANPFVQYEKETGDIIRKYSKKHNGPRIDKLKYKDGEVGACIDISHKYGFEKGSKKVILESLVPYRMDVYYKEENHSYYLVGVKQSDIKFEKGRNVIDEEAYARILVNEKMIQPGQSRADLENLGFKFKLSFYKNDIIEYEKDGKIYTERLVSRTMPKQRNYIETKPIDKAKFEKQNLVGLGKTKFIKKYRYDILGNKYSCSEEKFTSFC

SEQ ID NO: 92 - protein, 1084 aa, Corynebacterium diphtheriae
MKYHVGIDVGTFSVGLAAIEVDDAGMPIKTLSLVSHIHDSGLDPDKIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDEPSDAFKAIREEIKRASGQPVPETATVGQMVTLCELGTLKLRGEGGVLSARLQQSDHAREIQEICRMQEIGQELYRKIIDVVFAAESPKGSASSRVGKDPLQPGKNRALKASDAFQRYRIAALIGNLRVRVDGEKRILSVEEKNLVFDHLVNLAPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLADGVDLYTARLQEFGIEPSWTPPAPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDHIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLEFLDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHEETIGKLSKVKLGSQLSVSDIDKASSEALWCALTREPDFDPKDGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFGDTRLRLRPLQMSKEGIKKESAPELSKIIDRPGWLPAVNKLFSEGNVTVVRRDSLGRVRLESTAHLPVTWKVQ

SEQ ID NO: 93 - protein, 1152 aa, Roseburia inulinivorans
MNAEHGKEGLLIMEENFQYRIGLDIGITSVGWAVLQNNSQDEPVRITDLGVRIFDVAENPKNGDALAAPRRDARTTRRRLRRRRHRLERIKFLLQENGLIEMDSFMERYYKGNLPDVYQLRYEGLDRKLKDEELAQVLIHIAKHRGFRSTRKAETKEKEGGAVLKATTENQKIMQEKGYRTVGEMLYLDEAFHTECLWNEKGYVLTPRNRPDDYKHTILRSMLVEEVHAIFAAQRAHGNQKATEGLEEAYVEIMTSQRSFDMGPGLQPDGKPSPYAMEGFGDRVGKCTFEKDEYRAPKATYTAELFVALQKINHTKLIDEFGTGRFFSEEERKTIIGLLLSSKELKYGTIRKKLNIDPSLKFNSLNYSAKKEGETEEERVLDTEKAKFASMFWTYEYSKCLKDRTEEMPVGEKADLFDRIGEILTAYKNDDSRSSRLKELGLSGEEIDGLLDLSPAKYQRVSLKAMRKMQPYLEDGLIYDKACEAAGYDFRALNDGNKKHLLKGEEINAIVNDITNPVVKRSVSQTIKVINAIIQKYGSPQAVNIELAREMSKNFQDRTNLEKEMKKRQQENERAKQQIIELGKQNPTGQDILKYRLWNDQGGYCLYSGKKIPLEELFDGGYDIDHILPYSITFDDSYRNKVLVTAQENRQKGNRTPYEYFGADEKRWEDYEASVRLLVRDYKKQQKLLKKNFTEEERKEFKERNLNDTKYITRVVYNMIRQNLELEPFNHPEKKKQVWAVNGAVTSYLRKRWGLMQKDRSTDRHHAMDAVVIACCTDGMIHKISRYMQGRELAYSRNFKFPDEETGEILNRDNFTREQWDEKFGVKVPLPWNSFRDELDIRLLNEDPKNFLLTHADVQRELDYPGWMYGEEESPIEEGRYINYIRPLFVSRMPNHKVTGSAHDATIRSARDYETRGVVITKVPLTDLKLNKDNEIEGYYDKDSDRLLYQALVRQLLLHGNDGKKAFAEDFHKPKADGTEGPVVRKVKIEKKQTSGVMVRGGTGIAANGEMVRIDVFRENGKYYFVPVYTADVVRKVLPNRAATHTKPYSEWRVMDDANFVFSLYSRDLIHVKSKKDIKTNLVNGGLLLQKEIFAYYTGADIATASIAGFANDSNFKFRGLGIQSLEIFEKCQVDILGNISVVRHENRQEFH

SEQ ID NO: 94 - protein, 1029 aa, Alicycliphilus denitrificans
MRSLRYRLALDLGSTSLGWALFRLDACNRPTAVIKAGVRIFSDGRNPKDGSSLAVTRRAARAMRRRRDRLLKRKTRMQAKLVEHGFFPADAGKRKALEQLNPYALRAKGLQEALLPGEFARALFHINQRRGFKSNRKTDKKDNDSGVLKKAIGQLRQQMAEQGSRTVGEYLWTRLQQGQGVRARYREKPYTTEEGKKRIDKSYDLYIDRAMIEQEFDALWAAQAAFNPTLFHEAARADLKDTLLHQRPLRPVKPGRCTLLPEEERAPLALPSTQRFRIHQEVNHLRLLDENLREVALTLAQRDAVVTALETKAKLSFEQIRKLLKLSGSVQFNLEDAKRTELKGNATSAALARKELFGAAWSGFDEALQDEIVWQLVTEEGEGALIAWLQTHTGVDEARAQAIVDVSLPEGYGNLSRKALARIVPALRAAVITYDKAVQAAGFDHHSQLGFEYDASEVEDLVHPETGEIRSVFKQLPYYGKALQRHVAFGSGKPEDPDEKRYGKIANPTVHIGLNQVRMVVNALIRRYGRPTEVVIELARDLKQSREQKVEAQRRQADNQRRNARIRRSIAEVLGIGEERVRGSDIQKWICWEELSFDAADRRCPYSGVQISAAMLLSDEVEVEHILPFSKTLDDSLNNRTVAMRQANRIKRNRTPWDARAEFEAQGWSYEDILQRAERMPLRKRYRFAPDGYERWLGDDKDFLARALNDTRYLSRVAAEYLRLVCPGTRVIPGQLTALLRGKFGLNDVLGLDGEKNRNDHRHHAVDACVIGVTDQGLMQRFATASAQARGDGLTRLVDGMPMPWPTYRDHVERAVRHIWVSHRPDHGFEGAMMEETSYGIRKDGSIKQRRKADGSAGREISNLIRIHEATQPLRHGVSADGQPLAYKGYVGGSNYCIEITVNDKGKWEGEVISTFRAYGVVRAGGMGRLRNPHEGQNGRKLIMRLVIGDSVRLEVDGAERTMRIVKISGSNGQIFMAPIHEANVDARNTDKQDAFTYTSKYAGSLQKAKTRRVTISPIGEVRDPGFKG

SEQ ID NO: 95 - protein, 1179 aa, Sphaerochaeta globosa
MSKKVSRRYEEQAQEICQRLGSRPYSIGLDLGVGSIGVAVAAYDPIKKQPSDLVFVSSRIFIPSTGAAERRQKRGQRNSLRHRANRLKFLWKLLAERNLMLSYSEQDVPDPARLRFEDAVVRANPYELRLKGLNEQLTLSELGYALYHIANHRGSSSVRTFLDEEKSSDDKKLEEQQAMTEQLAKEKGISTFIEVLTAFNTNGLIGYRNSESVKSKGVPVPTRDIISNEIDVLLQTQKQFYQEILSDEYCDRIVSAILFENEKIVPEAGCCPYFPDEKKLPRCHFLNEERRLWEAINNARIKMPMQEGAAKRYQSASFSDEQRHILFHIARSGTDITPKLVQKEFPALKTSIIVLQGKEKAIQKIAGFRFRRLEEKSFWKRLSEEQKDDFFSAWTNTPDDKRLSKYLMKHLLLTENEVVDALKTVSLIGDYGPIGKTATQLLMKHLEDGLTYTEALERGMETGEFQELSVWEQQSLLPYYGQILTGSTQALMGKYWHSAFKEKRDSEGFFKPNTNSDEEKYGRIANPVVHQTLNELRKLMNELITILGAKPQEITVELARELKVGAEKREDIIKQQTKQEKEAVLAYSKYCEPNNLDKRYIERFRLLEDQAFVCPYCLEHISVADIAAGRADVDHIFPRDDTADNSYGNKVVAHRQCNDIKGKRTPYAAFSNTSAWGPIMHYLDETPGMWRKRRKFETNEEEYAKYLQSKGFVSRFESDNSYIAKAAKEYLRCLFNPNNVTAVGSLKGMETSILRKAWNLQGIDDLLGSRHWSKDADTSPTMRKNRDDNRHHGLDAIVALYCSRSLVQMINTMSEQGKRAVEIEAMIPIPGYASEPNLSFEAQRELFRKKILEFMDLHAFVSMKTDNDANGALLKDTVYSILGADTQGEDLVFVVKKKIKDIGVKIGDYEEVASAIRGRITDKQPKWYPMEMKDKIEQLQSKNEAALQKYKESLVQAAAVLEESNRKLIESGKKPIQLSEKTISKKALELVGGYYYLISNNKRTKTFVVKEPSNEVKGFAFDTGSNLCLDFYHDAQGKLCGEIIRKIQAMNPSYKPAYMKQGYSLYVRLYQGDVCELRASDLTEAESNLAKTTHVRLPNAKPGRTFVIIITFTEMGSGYQIYFSNLAKSKKGQDTSFTLTTIKNYDVRKVQLSSAGLVRYVSPLLVDKIEKDEVALCGE

SEQ ID NO: 96 - protein, 1374 aa, Fusobacterium nucleatum
MKKQKFSDYYLGFDIGTNSVGWCVTDLDYNVLRFNKKDMWGSRLFDEAKTAAERRVQRNSRRRLKRRKWRLNLLEEIFSDEIMKIDSNFFRRLKESSLWLEDKNSKEKFTLFNDDNYKDYDFYKQYPTIFHLRDELIKNPEKKDIRLIYLALHSIFKSRGHFLFEGQNLKEIKNFETLYNNLISFLEDNGINKSIDKDNIEKLEKIICDSGKGLKDKEKEFKGIFNSDKQLVAIFKLSVGSSVSLNDLFDTDEYKKEEVEKEKISFREQIYEDDKPIYYSILGEKIELLDIAKSFYDFMVLNNILSDSNYISEAKVKLYEEHKKDLKNLKYIIRKYNKENYDKLFKDKNENNYPAYIGLNKEKDKKEVVEKSRLKIDDLIKVIKGYLPKPERIEEKDKTIFNEILNKIELKTILPKQRISDNGTLPYQIHEVELEKILENQSKYYDFLNYEENGVSTKDKLLKTFKFRIPYYVGPLNSYHKDKGGNSWIVRKEEGKILPWNFEQKVDIEKSAEEFIKRMTNKCTYLNGEDVIPKDSFLYSEYIILNELNKVQVNDEFLNEENKRKIIDELFKENKKVSEKKFKEYLLVNQIANRTVELKGIKDSFNSNYVSYIKFKDIFGEKLNLDIYKEISEKSILWKCLYGDDKKIFEKKIKNEYGDILNKDEIKKINSFKFNTWGRLSEKLLTGIEFINLETGECYSSVMEALRRTNYNLMELLSSKFTLQESIDNENKEMNEVSYRDLIEESYVSPSLKRAILQTLKIYEEIKKITGRVPKKVFIEMARGGDESMKNKKIPARQEQLKKLYDSCGNDIANFSIDIKEMKNSLSSYDNNSLRQKKLYLYYLQFGKCMYTGREIDLDRLLQNNDTYDIDHIYPRSKVIKDDSFDNLVLVLKNENAEKSNEYPVKKEIQEKMKSFWRFLKEKNFISDEKYKRLTGKDDFELRGFMARQLVNVRQTTKEVGKILQQIEPEIKIVYSKAEIASSFREMFDFIKVRELNDTHHAKDAYLNIVAGNVYNTKFTEKPYRYLQEIKENYDVKKIYNYDIKNAWDKENSLEIVKKNMEKNTVNITRFIKEEKGELFNLNPIKKGETSNEIISIKPKLYDGKDNKLNEKYGYYTSLKAAYFIYVEHEKKNKKVKTFERITRIDSTLIKNEKNLIKYLVSQKKLLNPKIIKKIYKEQTLIIDSYPYTFTGVDSNKKVELKNKKQLYLEKKYEQILKNALKFVEDNQGETEENYKFIYLKKRNNNEKNETIDAVKERYNIEFNEMYDKFLEKLSSKDYKNYINNKLYTNFLNSKEKFKKLKLWEKSLILREFLKIFNKNTYGKYEIKDSQTKEKLFSFPEDTGRIRLGQSSLGNNKELLEESVTGLFVKKIKL

SEQ ID NO: 97 - protein, 1056 aa, Pasteurella multocida
MQTTNLSYILGLDLGIASVGWAVVEINENEDPIGLIDVGVRIFERAEVPKTGESLALSRRLARSTRRLIRRRAHRLLLAKRFLKREGILSTIDLEKGLPNQAWELRVAGLERRLSAIEWGAVLLHLIKHRGYLSKRKNESQTNNKELGALLSGVAQNHQLLQSDDYRTPAELALKKFAKEEGHIRNQRGAYTHTFNRLDLLAELNLLFAQQHQFGNPHCKEHIQQYMTELLMWQKPALSGEAILKMLGKCTHEKNEFKAAKHTYSAERFVWLTKLNNLRILEDGAERALNEEERQLLINHPYEKSKLTYAQVRKLLGLSEQAIFKHLRYSKENAESATFMELKAWHAIRKALENQGLKDTWQDLAKKPDLLDEIGTAFSLYKTDEDIQQYLTNKVPNSVINALLVSLNFDKFIELSLKSLRKILPLMEQGKRYDQACREIYGHHYGEANQKTSQLLPAIPAQEIRNPVVLRTLSQARKVINAIIRQYGSPARVHIETGRELGKSFKERREIQKQQEDNRTKRESAVQKFKELFSDFSSEPKSKDILKFRLYEQQHGKCLYSGKEINIHRLNEKGYVEIDHALPFSRTWDDSFNNKVLVLASENQNKGNQTPYEWLQGKINSERWKNFVALVLGSQCSAAKKQRLLTQVIDDNKFIDRNLNDTRYIARFLSNYIQENLLLVGKNKKNVFTPNGQITALLRSRWGLIKARENNNRHHALDAIVVACATPSMQQKITRFIRFKEVHPYKIENRYEMVDQESGEIISPHFPEPWAYFRQEVNIRVFDNHPDTVLKEMLPDRPQANHQFVQPLFVSRAPTRKMSGQGHMETIKSAKRLAEGISVLRIPLTQLKPNLLENMVNKEREPALYAGLKARLAEFNQDPAKAFATPFYKQGGQQVKAIRVEQVQKSGVLVRENNGVADNASIVRTDVFIKNNKFFLVPIYTWQVAKGILPNKAIVAHKNEDEWEEMDEGAKFKFSLFPNDLVELKTKKEYFFGYYIGLDRATGNISLKEHDGEISKGKDGVYRVGVKLALSFEKYQVDELGKNRQICRPQQRQPVR

SEQ ID NO: 98 - protein, 1113 aa, Alcanivorax pacificus
MRYRVGLDLGTASVGAAVFSMDEQGNPMELIWHYERLFSEPLVPDMGQLKPKKAARRLARQQRRQIDRRASRLRRIAIVSRRLGIAPGRNDSGVHGNDVPTLRAMAVNERIELGQLRAVLLRMGKKRGYGGTFKAVRKVGEAGEVASGASRLEEEMVALASVQNKDSVTVGEYLAARVEHGLPSKLKVAANNEYYAPEYALFRQYLGLPAIKGRPDCLPNMYALRHQIEHEFERIWATQSQFHDVMKDHGVKEEIRNAIFFQRPLKSPADKVGRCSLQTNLPRAPRAQIAAQNFRIEKQMADLRWGMGRRAEMLNDHQKAVIRELLNQQKELSFRKIYKELERAGCPGPEGKGLNMDRAALGGRDDLSGNTTLAAWRKLGLEDRWQELDEVTQIQVINFLADLGSPEQLDTDDWSCRFMGKNGRPRNFSDEFVAFMNELRMTDGFDRLSKMGFEGGRSSYSIKALKALTEWMIAPHWRETPETHRVDEEAAIRECYPESLATPAQGGRQSKLEPPPLTGNEVVDVALRQVRHTINMMIDDLGSVPAQIVVEMAREMKGGVTRRNDIEKQNKRFASERKKAAQSIEENGKTPTPARILRYQLWIEQGHQCPYCESNISLEQALSGAYTNFEHILPRTLTQIGRKRSELVLAHRECNDEKGNRTPYQAFGHDDRRWRIVEQRANALPKKSSRKTRLLLLKDFEGEALTDESIDEFADRQLHESSWLAKVTTQWLSSLGSDVYVSRGSLTAELRRRWGLDTVIPQVRFESGMPVVDEEGAEITPEEFEKFRLQWEGHRVTREMRTDRRPDKRIDHRHHLVDAIVTALTSRSLYQQYAKAWKVADEKQRHGRVDVKVELPMPILTIRDIALEAVRSVRISHKPDRYPDGRFFEATAYGIAQRLDERSGEKVDWLVSRKSLTDLAPEKKSIDVDKVRANISRIVGEAIRLHISNIFEKRVSKGMTPQQALREPIEFQGNILRKVRCFYSKADDCVRIEHSSRRGHHYKMLLNDGFAYMEVPCKEGILYGVPNLVRPSEAVGIKRAPESGDFIRFYKGDTVKNIKTGRVYTIKQILGDGGGKLILTPVTETKPADLLSAKWGRLKVGGRNIHLLRLCAE

SEQ ID NO: 99 - protein, 1236 aa, Mycoplasma mobile
MYFYKNKENKLNKKVVLGLDLGIASVGWCLTDISQKEDNKFPIILHGVRLFETVDDSDDKLLNETRRKKRGQRRRNRRLFTRKRDFIKYLIDNNIIELEFDKNPKILVRNFIEKYINPFSKNLELKYKSVTNLPIGFHNLRKAAINEKYKLDKSELIVLLYFYLSLRGAFFDNPEDTKSKEMNKNEIEIFDKNESIKNAEFPIDKIIEFYKISGKIRSTINLKFGHQDYLKEIKQVFEKQNIDFMNYEKFAMEEKSFFSRIRNYSEGPGNEKSFSKYGLYANENGNPELIINEKGQKIYTKIFKTLWESKIGKCSYDKKLYRAPKNSFSAKVFDITNKLTDWKHKNEYISERLKRKILLSRFLNKDSKSAVEKILKEENIKFENLSEIAYNKDDNKINLPIINAYHSLTTIFKKHLINFENYLISNENDLSKLMSFYKQQSEKLFVPNEKGSYEINQNNNVLHIFDAISNILNKFSTIQDRIRILEGYFEFSNLKKDVKSSEIYSEIAKLREFSGTSSLSFGAYYKFIPNLISEGSKNYSTISYEEKALQNQKNNFSHSNLFEKTWVEDLIASPTVKRSLRQTMNLLKEIFKYSEKNNLEIEKIVVEVTRSSNNKHERKKIEGINKYRKEKYEELKKVYDLPNENTTLLKKLWLLRQQQGYDAYSLRKIEANDVINKPWNYDIDHIVPRSISFDDSFSNLVIVNKLDNAKKSNDLSAKQFIEKIYGIEKLKEAKENWGNWYLRNANGKAFNDKGKFIKLYTIDNLDEFDNSDFINRNLSDTSYITNALVNHLTFSNSKYKYSVVSVNGKQTSNLRNQIAFVGIKNNKETEREWKRPEGFKSINSNDFLIREEGKNDVKDDVLIKDRSFNGHHAEDAYFITIISQYFRSFKRIERLNVNYRKETRELDDLEKNNIKFKEKASFDNFLLINALDELNEKLNQMRFSRMVITKKNTQLFNETLYSGKYDKGKNTIKKVEKLNLLDNRTDKIKKIEEFFDEDKLKENELTKLHIFNHDKNLYETLKIIWNEVKIEIKNKNLNEKNYFKYFVNKKLQEGKISFNEWVPILDNDFKIIRKIRYIKFSSEEKETDEIIFSQSNFLKIDQRQNFSFHNTLYWVQIWVYKNQKDQYCFISIDARNSKFEKDEIKINYEKLKTQKEKLQIINEEPILKINKGDLFENEEKELFYIVGRDEKPQKLEIKYILGKKIKDQKQIQKPVKKYFPNWKKVNLTYMGEIFKK

SEQ ID NO: 100 - protein, 1397 aa, unidentified
MTKNYISPIAIDLGAKFTGVALYQYLEGADCTQEVAKGLLVDDRGNVTWSQEGRRGKRHQVRGYKRRKMAKRLLWLILDSEYGIKREEVTEPLLKFINGLLNRRGYTYISEEVDEESMNVSPLPFSEMMPDYFNSSAPLLEQLAKLLSDKNKLVRFRAEGKIPSNKNEFKKLLDTALDGKYKDEKKELSEAWGNILIASENVLKSTVDGHKSRSEYLANIKEDIKSNEELEKQISSKEIDGFYNLVGHLSNFQLRLLRKYFNDPNMSGVSYWDEKRLEKYFYQWVQGWHTKGGTDEAEKKNIILKTKGAPLLKTLKSLSADLTIPPYEDQNNRRPPKCQSVLLSDEKLTMHYPKWKEWVGQLVKQNDNAYLNENVTLANALHRIVERSRSIDPYQLRLLISITDAEKRNDLAGYKRLKLSLGSEVDEFLLLVKNIVDETKEAREGLWFETENKLFFKCGKTPPRKEKLKSTLLSAVLGKNLSDDEQSSFIEEFWKSGTPKIERRNVRGWCRLASQVQKTYGVYLKEYGLQQLHKLEAGKKLDDKPLALLYKNSGLIASKIGEALNIEPDEVSRFASPHSLAQIFNIIEGDVAGFNKTCRACTYENIWRMQEEKVESLLTNQLLSEIHGERKVPLKSAMCTRLSADSTRPFDGQMASIIEHIARKIAQHKIAQINDVPKEFSIDIPIIIESNQFSFTAELEEIKRGRGSAKAKKAKELGEKSKAGWVSKTERIKTSSEGICPYTGAPLGGSGEIDHIIPRSLTGRTKKTVFNSEANLIYCSSKGNHDKGNRVYVIEQLNDKYLKKQFSTSDVNLIKKKIKTTIQRFTEGGEKLRSFSELSREDQKAFRHALFVPELKSEVTSLLAVKNITRVNGTQAWLAKKIASLLAEHLDKQGRDYTLSAHQIDPWSVSKQRKMLASAEPIWAKKDPQPAASHVVDAVCTFLEALEQPHTASRLKTISSTSFEKTGWRSALIPDLIKVDALDRRPKYRRYNIGSTSLFKDGIYAERFLPILIDENGLMAGYDIDNSLKAKGADVVFESLSPFLLFKGEEVGAQSLSDWQERIDGRYLYMSIDKVKAFDYLQEKVGEKDIAAELLNSIHFTQRKTELRAKFSDDSGKKMKTLDAIRKSLKLTVTVNEIGKRKEKCGFSGTIGIPAKSAWENLLDEPLLETYWGTKMPPQEIWEKVYRKHFPRNIPNQAHRKVRKDFSLPVVDSVSGGFRVKRKTPNGYNYQLLAIDGYSAVGFKKEGDNVDFKSPALVPQIAESKSVTPISSELVHLDKNEIVYFDEWRKIDISDSDLKQFVSSLELAPGSQNRFYIRFTVDEDQFERHFKSALRVNGIQDLDTVNKTFDWNREIPSLLIPPRSNLFLLETGQKITFEYIANGANAEVKKAYSLRRA

SEQ ID NO: 101 - protein, 1333 aa, Planococcus antarcticus
MKNYTIGLDIGVASVGWVCIDENYKILNYNNRHAFGVHEFESAESAAGRRLKRGMRRRYNRRKKRLQLLQSLFDSYITDSGFFSKTDSQHFWKNNNEFENRSLTEVLSSLRISSRKYPTIYHLRSDLIESNKKMDLRLVYLALHNLVKYRGHFLQEGNWSEAASAEGMDDQLLELVTRYAELENLSPLDLSESQWKAAETLLLNRNLTKTDQSKELTAMFGKEYEPFCKLVAGLGVSLHQLFPSSEQALAYKETKTKVQLSNENVEEVMELLLEEESALLEAVQPFYQQVVLYELLKGETYVAKAKVSAFKQYQKDMASLKNLLDKTFGEKVYRSYFISDKNSQREYQKSHKVEVLCKLDQFNKEAKFAETFYKDLKKLLEDKSKTSIGTTEKDEMLRIIKAIDSNQFLQKQKGIQNAAIPHQNSLYEAEKILRNQQAHYPFITTEWIEKVKQILAFRIPYYIGPLVKDTTQSPFSWVERKGDAPITPWNFDEQIDKAASAEAFISRMRKTCTYLKGQEVLPKSSLTYERFEVLNELNGIQLRTTGAESDFRHRLSYEMKCWIIDNVFKQYKTVSTKRLLQELKKSPYADELYDEHTGEIKEVFGTQKENAFATSLSGYISMKSILGAVVDDNPAMTEELIYWIAVFEDREILHLKIQEKYPSITDVQRQKLALVKLPGWGRFSRLLIDGLPLDEQGQSVLDHMEQYSSVFMEVLKNKGFGLEKKIQKMNQHQVDGTKKIRYEDIEELAGSPALKRGIWRSVKIVEELVSIFGEPANIVLEVAREDGEKKRTKSRKDQWEELTKTTLKNDPDLKSFIGEIKSQGDQRFNEQRFWLYVTQQGKCLYTGKALDIQNLSMYEVDHILPQNFVKDDSLDNLALVMPEANQRKNQVGQNKMPLEIIEANQQYAMRTLWERLHELKLISSGKLGRLKKPSFDEVDKDKFIARQLVETRQIIKHVRDLLDERFSKSDIHLVKAGIVSKFRRFSEIPKIRDYNNKHHAMDALFAAALIQSILGKYGKNFLAFDLSKKDRQKQWRSVKGSNKEFFLFKNFGNLRLQSPVTGEEVSGVEYMKHVYFELPWQTTKMTQTGDGMFYKESIFSPKVKQAKYVSPKTEKFVHDEVKNHSICLVEFTFMKKEKEVQETKFIDLKVIEHHQFLKEPESQLAKFLAEKETNSPIIHARIIRTIPKYQKIWIEHFPYYFISTRELHNARQFEISYELMEKVKQLSERSSVEELKIVFGLLIDQMNDNYPIYTKSSIQDRVQKFVDTQLYDFKSFEIGFEELKKAVAANAQRSDTFGSRISKKPKPEEVAIGYESITGLKYRKPRSVVGTKR

SEQ ID NO: 102 - protein, 1424 aa, Prevotella sp.
MTQKVLGLDLGTNSIGSAVRNLDLSDDLQWQLEFFSSDIFRSSVNKESNGREYSLAAQRSAHRRSRGLNEVRRRRLWATLNLLIKHGFCPMSSESLMRWCTYDKRKGLFREYPIDDKDFNAWILLDFNGDGRPDYSSPYQLRRELVTRQFDFEQPIERYKLGRALYHIAQHRGFKSSKGETLSQQETNSKPSSTDEIPDVAGAMKASEEKLSKGLSTYMKEHNLLTVGAAFAQLEDEGVRVRNNNDYRAIRSQFQHEIETIFKFQQGLSVESELYERLISEKKNVGTIFYKRPLRSQRGNVGKCTLERSKPRCAIGHPLFEKFRAWTLINNIKVRMSVDTLDEQLPMKLRLDLYNECFLAFVRTEFKFEDIRKYLEKRLGIHFSYNDKTINYKDSTSVAGCPITARFRKMLGEEWESFRVEGQKERQAHSKNNISFHRVSYSIEDIWHFCYDAEEPEAVLAFAQETLRLERKKAEELVRIWSAMPQGYAMLSQKAIRNINKILMLGLKYSDAVILAKVPELVDVSDEELLSIAKDYYLVEAQVNYDKRINSIVNGLIAKYKSVSEEYRFADHNYEYLLDESDEKDIIRQIENSLGARRWSLMDANEQTDILQKVRDRYQDFFRSHERKFVESPKLGESFENYLTKKFPMVEREQWKKLYHPSQITIYRPVSVGKDRSVLRLGNPDIGAIKNPTVLRVLNTLRRRVNQLLDDGVISPDETRVVVETARELNDANRKWALDTYNRIRHDENEKIKKILEEFYPKRDGISTDDIDKARYVIDQREVDYFTGSKTYNKDIKKYKFWLEQGGQCMYTGRTINLSNLFDPNAFDIEHTIPESLSFDSSDMNLTLCDAHYNRFIKKNHIPTDMPNYDKAITIDGKEYPAITSQLQRWVERVERLNRNVEYWKGQARRAQNKDRKDQCMREMHLWKMELEYWKKKLERFTVTEVTDGFKNSQLVDTRVITRHAVLYLKSIFPHVDVQRGDVTAKFRKILGIQSVDEKKDRSLHSHHAIDATTLTIIPVSAKRDRMLELFAKIEEINKMLSFSGSEDRTGLIQELEGLKNKLQMEVKVCRIGHNVSEIGTFINDNIIVNHHIKNQALTPVRRRLRKKGYIVGGVDNPRWQTGDALRGEIHKASYYGAITQFAKDDEGKVLMKEGRPQVNPTIKFVIRRELKYKKSAADSGFASWDDLGKAIVDKELFALMKGQFPAETSFKDACEQGIYMIKKGKNGMPDIKLHHIRHVRCEAPQSGLKIKEQTYKSEKEYKRYFYAAVGDLYAMCCYTNGKIREFRIYSLYDVSCHRKSDIEDIPEFITDKKGNRLMLDYKLRTGDMILLYKDNPAELYDLDNVNLSRRLYKINRFESQSNLVLMTHHLSTSKERGRSLGKTVDYQNLPESIRSSVKSLNFLIMGENRDFVIKNGKIIFNHR

SEQ ID NO: 103 - protein, 1146 aa, Alicyclobacillus hesperidum
MAYRLGLDIGITSVGWAVVALEKDESGLKPVRIQDLGVRIFDKAEDSKTGASLALPRREARSARRRTRRRRHRLWRVKRLLEQHGILSMEQIEALYAQRTSSPDVYALRVAGLDRCLIAEEIARVLIHIAHRRGFQSNRKSEIKDSDAGKLLKAVQENENLMQSKGYRTVAEMLVSEATKTDAEGKLVHGKKHGYVSNVRNKAGEYRHTVSRQAIVDEVRKIFAAQRALGNDVMSEELEDSYLKILCSQRNFDDGPGGDSPYGHGSVSPDGVRQSIYERMVGSCTFETGEKRAPRSSYSFERFQLLTKVVNLRIYRQQEDGGRYPCELTQTERARVIDCAYEQTKITYGKLRKLLDMKDTESFAGLTYGLNRSRNKTEDTVFVEMKFYHEVRKALQRAGVFIQDLSIETLDQIGWILSVWKSDDNRRKKLSTLGLSDNVIEELLPLNGSKFGHLSLKAIRKILPFLEDGYSYDVACELAGYQFQGKTEYVKQRLLPPLGEGEVTNPVVRRALSQAIKVVNAVIRKHGSPESIHIELARELSKNLDERRKIEKAQKENQKNNEQIKDEIREILGSAHVTGRDIVKYKLFKQQQEFCMYSGEKLDVTRLFEPGYAEVDHIIPYGISFDDSYDNKVLVKTEQNRQKGNRTPLEYLRDKPEQKAKFIALVESIPLSQKKKNHLLMDKRAIDLEQEGFRERNLSDTRYITRALMNHIQAWLLFDETASTRSKRVVCVNGAVTAYMRARWGLTKDRDAGDKHHAADAVVVACIGDSLIQRVTKYDKFKRNALADRNRYVQQVSKSEGITQYVDKETGEVFTWESFDERKFLPNEPLEPWPFFRDELLARLSDDPSKNIRAIGLLTYSETEQIDPIFVSRMPTRKVTGAAHKETIRSPRIVKVDDNKGTEIQVVVSKVALTELKLTKDGEIKDYFRPEDDPRLYNTLRERLVQFGGDAKAAFKEPVYKISKDGSVRTPVRKVKIQEKLTLGVPVHGGRGIAENGGMVRIDVFAKGGKYYFVPIYVADVLKRELPNRLATAHKPYSEWRVVDDSYQFKFSLYPNDAVMIKPSREVDITYKDRKEPVGCRIMYFVSANIASASISLRTHDNSGELEGLGIQGLEVFEKYVVGPLGDTHPVYKERRMPFRVERKMN

SEQ ID NO: 104 - protein, 1363 aa, Lactobacillus rhamnosus
MTKLNQPYGIGLDIGSNSIGFAVVDANSHLLRLKGETAIGARLFREGQSAADRRGSRTTRRRLSRTRWRLSFLRDFFAPHITKIDPDFFLRQKYSEISPKDKDRFKYEKRLFNDRTDAEFYEDYPSMYHLRLHLMTHTHKADPREIFLAIHHILKSRGHFLTPGAAKDFNTDKVDLEDIFPALTEAYAQVYPDLELTFDLAKADDFKAKLLDEQATPSDTQKALVNLLLSSDGEKEIVKKRKQVLTEFAKAITGLKTKFNLALGTEVDEADASNWQFSMGQLDDKWSNIETSMTDQGTEIFEQIQELYRARLLNGIVPAGMSLSQAKVADYGQHKEDLELFKTYLKKLNDHELAKTIRGLYDRYINGDDAKPFLREDFVKALTKEVTAHPNEVSEQLLNRMGQANFMLKQRTKANGAIPIQLQQRELDQIIANQSKYYDWLAAPNPVEAHRWKMPYQLDELLNFHIPYYVGPLITPKQQAESGENVFAWMVRKDPSGNITPYNFDEKVDREASANTFIQRMKTTDTYLIGEDVLPKQSLLYQKYEVLNELNNVRINNECLGTDQKQRLIREVFERHSSVTIKQVADNLVAHGDFARRPEIRGLADEKRFLSSLSTYHQLKEILHEAIDDPTKLLDIENIITWSTVFEDHTIFETKLAEIEWLDPKKINELSGIRYRGWGQFSRKLLDGLKLGNGHTVIQELMLSNHNLMQILADETLKETMTELNQDKLKTDDIEDVINDAYTSPSNKKALRQVLRVVEDIKHAANGQDPSWLFIETADGTGTAGKRTQSRQKQIQTVYANAAQELIDSAVRGELEDKIADKASFTDRLVLYFMQGGRDIYTGAPLNIDQLSHYDIDHILPQSLIKDDSLDNRVLVNATINREKNNVFASTLFAGKMKATWRKWHEAGLISGRKLRNLMLRPDEIDKFAKGFVARQLVETRQIIKLTEQIAAAQYPNTKIIAVKAGLSHQLREELDFPKNRDVNHYHHAFDAFLAARIGTYLLKRYPKLAPFFTYGEFAKVDVKKFREFNFIGALTHAKKNIIAKDTGEIVWDKERDIRELDRIYNFKRMLITHEVYFETADLFKQTIYAAKDSKERGGSKQLIPKKQGYPTQVYGGYTQESGSYNALVRVAEADTTAYQVIKISAQNASKIASANLKSREKGKQLLNEIVVKQLAKRRKNWKPSANSFKIVIPRFGMGTLFQNAKYGLFMVNSDTYYRNYQELWLSRENQKLLKKLFSIKYEKTQMNHDALQVYKAIIDQVEKFFKLYDINQFRAKLSDAIERFEKLPINTDGNKIGKTETLRQILIGLQANGTRSNVKNLGIKTDLGLLQVGSGIKLDKDTQIVYQSPSGLFKRRIPLADL

SEQ ID NO: 105 - protein, 1150 aa, Enterococcus faecalis
MYSIGLDLGISSVGWSVIDERTGNVIDLGVRLFSAKNSEKNLERRTNRGGRRLIRRKTNRLKDAKKILAAVGFYEDKSLKNSCPYQLRVKGLTEPLSRGEIYKVTLHILKKRGISYLDEVDTEAAKESQDYKEQVRKNAQLLTKYTPGQIQLQRLKENNRVKTGINAQGNYQLNVFKVSAYANELATILKTQQAFYPNELTDDWIALFVQPGIAEEAGLIYRKRPYYHGPGNEANNSPYGRWSDFQKTGEPATNIFDKLIGKDFQGELRASGLSLSAQQYNLLNDLTNLKIDGEVPLSSEQKEYILTELMTKEFTRFGVNDVVKLLGVKKERLSGWRLDKKGKPEIHTLKGYRNWRKIFAEAGIDLATLPTETIDCLAKVLTLNTEREGIENTLAFELPELSESVKLLVLDRYKELSQSISTQSWHRFSLKTLHLLIPELMNATSEQNTLLEQFQLKSDVRKRYSEYKKLPTKDVLAEIYNPTVNKTVSQAFKVIDALLVKYGKEQIRYITIEMPRDDNEEDEKKRIKELHAKNSQRKNDSQSYFMQKSGWSQEKFQTTIQKNRRFLAKLLYYYEQDGICAYTGLPISPELLVSDSTEIDHIIPISISLDDSINNKVLVLSKANQVKGQQTPYDAWMDGSFKKINGKFSNWDDYQKWVESRHFSHKKENNLLETRNIFDSEQVEKFLARNLNDTRYASRLVLNTLQSFFTNQETKVRVVNGSFTHTLRKKWGADLDKTRETHHHHAVDATLCAVTSFVKVSRYHYAVKEETGEKVMREIDFETGEIVNEMSYWEFKKSKKYERKTYQVKWPNFREQLKPVNLHPRIKFSHQVDRKANRKLSDATIYSVREKTEVKTLKSGKQKITTDEYTIGKIKDIYTLDGWEAFKKKQDKLLMKDLDEKTYERLLSIAETTPDFQEVEEKNGKVKRVKRSPFAVYCEENDIPAIQKYAKKNNGPLIRSLKYYDGKLNKHINITKDSQGRPVEKTKNGRKVTLQSLKPYRYDIYQDLETKAYYTVQLYYSDLRFVEGKYGITEKEYMKKVAEQTKGQVVRFCFSLQKNDGLEIEWKDSQRYDVRFYNFQSANSINFKGLEQEMMPAENQFKQKPYNNGAINLNIAKYGKEGKKLRKFNTDILGKKHYLFYEKEPKNIIK

SEQ ID NO: 106 - protein, 1035 aa, unidentified
MRRLGLDLGTNSIGWCLLDLGDDGEPVSIFRTGARIFSDGRDPKSLGSLKATRREARLTRRRRDRFIQRQKNLINALVKYGLMPADEIQRQALAYKDPYPIRKKALDEAIDPYEMGRAIFHINQRRGFKSNRKSADNEAGVVKQSIADLEMKLGEAGARTIGEFLADRQATNDTVRARRLSGTNALYEFYPDRYMLEQEFDTLWAKQAAFNPSLYIEAARERLKEIVFFQRKLKPQEVGRCIFLSDEDRISKALPSFQRFRIYQELSNLAWIDHDGVAHRITASLALRDHLFDELEHKKKLTFKAMRAILRKQGVVDYPVGFNLESDNRDHLIGNLTSCIMRDAKKMIGSAWDRLDEEEQDSFILMLQDDQKGDDEVRSILTQQYGLSDDVAEDCLDVRLPDGHGSLSKKAIDRILPVLRDQGLIYYDAVKEAGLGEANLYDPYAALSDKLDYYGKALAGHVMGASGKFEDSDEKRYGTISNPTVHIALNQVRAVVNELIRLHGKPDEVVIEIGRDLPMGADGKRELERFQKEGRAKNERARDELKKLGHIDSRESRQKFQLWEQLAKEPVDRCCPFTGKMMSISDLFSDKVEIEHLLPFSLTLDDSMANKTVCFRQANRDKGNRAPFDAFGNSPAGYDWQEILGRSQNLPYAKRWRFLPDAMKRFEADGGFLERQLNDTRYISRYTTEYISTIIPKNKIWVVTGRLTSLLRGFWGLNSILRGHNTDDGTPAKKSRDDHRHHAIDAIVVGMTSRGLLQKVSKAARRSEDLDLTRLFEGRIDPWDGFRDEVKKHIDAIIVSHRPRKKSQGALHNDTAYGIVEHAENGASTVVHRVPITSLGKQSDIEKVRDPLIKSALLNETAGLSGKSFENAVQKWCADNSIKSLRIVETVSIIPITDKEGVAYKGYKGDGNAYMDIYQDPTSSKWKGEIVSRFDANQKGFIPSWQSQFPTARLIMRLRINDLLKLQDGEIEEIYRVQRLSGSKILMAPHTEANVDARDRDKNDTFKLTSKSPGKLQSASARKVHISPTGLIREG

SEQ ID NO: 107 - protein, 1389 aa, Oenococcus kitaharae
MARDYSVGLDIGTSSVGWAAIDNKYHLIRAKSKNLIGVRLFDSAVTAEKRRGYRTTRRRLSRRHWRLRLLNDIFAGPLTDFGDENFLARLKYSWVHPQDQSNQAHFAAGLLFDSKEQDKDFYRKYPTIYHLRLALMNDDQKHDLREVYLAIHHLVKYRGHFLIEGDVKADSAFDVHTFADAIQRYAESNNSDENLLGKIDEKKLSAALTDKHGSKSQRAETAETAFDILDLQSKKQIQAILKSVVGNQANLMAIFGLDSSAISKDEQKNYKFSFDDADIDEKIADSEALLSDTEFEFLCDLKAAFDGLTLKMLLGDDKTVSAAMVRRFNEHQKDWEYIKSHIRNAKNAGNGLYEKSKKFDGINAAYLALQSDNEDDRKKAKKIFQDEISSADIPDDVKADFLKKIDDDQFLPIQRTKNNGTIPHQLHRNELEQIIEKQGIYYPFLKDTYQENSHELNKITALINFRVPYYVGPLVEEEQKIADDGKNIPDPTNHWMVRKSNDTITPWNLSQVVDLDKSGRRFIERLTGTDTYLIGEPTLPKNSLLYQKFDVLQELNNIRVSGRRLDIRAKQDAFEHLFKVQKTVSATNLKDFLVQAGYISEDTQIEGLADVNGKNFNNALTTYNYLVSVLGREFVENPSNEELLEEITELQTVFEDKKVLRRQLDQLDGLSDHNREKLSRKHYTGWGRISKKLLTTKIVQNADKIDNQTFDVPRMNQSIIDTLYNTKMNLMEIINNAEDDFGVRAWIDKQNTTDGDEQDVYSLIDELAGPKEIKRGIVQSFRILDDITKAVGYAPKRVYLEFARKTQESHLTNSRKNQLSTLLKNAGLSELVTQVSQYDAAALQNDRLYLYFLQQGKDMYSGEKLNLDNLSNYDIDHIIPQAYTKDNSLDNRVLVSNITNRRKSDSSNYLPALIDKMRPFWSVLSKQGLLSKHKFANLTRTRDFDDMEKERFIARSLVETRQIIKNVASLIDSHFGGETKAVAIRSSLTADMRRYVDIPKNRDINDYHHAFDALLFSTVGQYTENSGLMKKGQLSDSAGNQYNRYIKEWIHAARLNAQSQRVNPFGFVVGSMRNAAPGKLNPETGEITPEENADWSIADLDYLHKVMNFRKITVTRRLKDQKGQLYDESRYPSVLHDAKSKASINFDKHKPVDLYGGFSSAKPAYAALIKFKNKFRLVNVLRQWTYSDKNSEDYILEQIRGKYPKAEMVLSHIPYGQLVKKDGALVTISSATELHNFEQLWLPLADYKLINTLLKTKEDNLVDILHNRLDLPEMTIESAFYKAFDSILSFAFNRYALHQNALVKLQAHRDDFNALNYEDKQQTLERILDALHASPASSDLKKINLSSGFGRLFSPSHFTLADTDEFIFQSVTGLFSTQKTVAQLYQETK

SEQ ID NO: 108 - protein, 1024 aa, Helicobacter mustelae
MIRTLGIDIGIASIGWAVIEGEYTDKGLENKEIVASGVRVFTKAENPKNKESLALPRTLARSARRRNARKKGRIQQVKHYLSKALGLDLECFVQGEKLATLFQTSKDFLSPWELRERALYRVLDKEELARVILHIAKRRGYDDITYGVEDNDSGKIKKAIAENSKRIKEEQCKTIGEMMYKLYFQKSLNVRNKKESYNRCVGRSELREELKTIFQIQQELKSPWVNEELIYKLLGNPDAQSKQEREGLIFYQRPLKGFGDKIGKCSHIKKGENSPYRACKHAPSAEEFVALTKSINFLKNLTNRHGLCFSQEDMCVYLGKILQEAQKNEKGLTYSKLKLLLDLPSDFEFLGLDYSGKNPEKAVFLSLPSTFKLNKITQDRKTQDKIANILGANKDWEAILKELESLQLSKEQIQTIKDAKLNFSKHINLSLEALYHLLPLMREGKRYDEGVEILQERGIFSKPQPKNRQLLPPLSELAKEESYFDIPNPVLRRALSEFRKVVNALLEKYGGFHYFHIELTRDVCKAKSARMQLEKINKKNKSENDAASQLLEVLGLPNTYNNRLKCKLWKQQEEYCLYSGEKITIDHLKDQRALQIDHAFPLSRSLDDSQSNKVLCLTSSNQEKSNKTPYEWLGSDEKKWDMYVGRVYSSNFSPSKKRKLTQKNFKERNEEDFLARNLVDTGYIGRVTKEYIKHSLSFLPLPDGKKEHIRIISGSMTSTMRSFWGVQEKNRDHHLHHAQDAIIIACIEPSMIQKYTTYLKDKETHRLKSHQKAQILREGDHKLSLRWPMSNFKDKIQESIQNIIPSHHVSHKVTGELHQETVRTKEFYYQAFGGEEGVKKALKFGKIREINQGIVDNGAMVRVDIFKSKDKGKFYAVPIYTYDFAIGKLPNKAIVQGKKNGIIKDWLEMDENYEFCFSLFKNDCIKIQTKEMQEAVLAIYKSTNSAKATIELEHLSKYALKNEDEEKMFTDTDKEKNKTMTRESCGIQGLKVFQKVKLSVLGEVLEHKPRNRQNIALKTTPKHV

SEQ ID NO: 109 - protein, 1064 aa, Bradyrhizobium sp.
MKRTSLRAYRLGVDLGANSLGWFVVWLDDHGQPEGLGPGGVRIFPDGRNPQSKQSNAAGRRLARSARRRRDRYLQRRGKLMGLLVKHGLMPADEPARKRLECLDPYGLRAKALDEVLPLHHVGRALFHLNQRRGLFANRAIEQGDKDASAIKAAAGRLQTSMQACGARTLGEFLNRRHQLRATVRARSPVGGDVQARYEFYPTRAMVDAEFEAIWAAQAPHHPTMTAEAHDTIREAIFSQRAMKRPSIGKCSLDPATSQDDVDGFRCAWSHPLAQRFRIWQDVRNLAVVETGPTSSRLGKEDQDKVARALLQTDQLSFDEIRGLLGLPSDARFNLESDRRDHLKGDATGAILSARRHFGPAWHDRSLDRQIDIVALLESALDEAAIIASLGTTHSLDEAAAQRALSALLPDGYCRLGLRAIKRVLPLMEAGRTYAEAASAAGYDHALLPGGKLSPTGYLPYYGQWLQNDVVGSDDERDTNERRWGRLPNPTVHIGIGQLRRVVNELIRWHGPPAEITVELTRDLKLSPRRLAELEREQAENQRKNDKRTSLLRKLGLPASTHNLLKLRLWDEQGDVASECPYTGEAIGLERLVSDDVDIDHLIPFSISWDDSAANKVVCMRYANREKGNRTPFEAFGHRQGRPYDWADIAERAARLPRGKRWRFGPGARAQFEELGDFQARLLNETSWLARVAKQYLAAVTHPHRIHVLPGRLTALLRATWELNDLLPGSDDRAAKSRKDHRHHAIDALVAALTDQALLRRMANAHDDTRRKIEVLLPWPTFRIDLETRLKAMLVSHKPDHGLQARLHEDTAYGTVEHPETEDGANLVYRKTFVDISEKEIDRIRDRRLRDLVRAHVAGERQQGKTLKAAVLSFAQRRDIAGHPNGIRHVRLTKSIKPDYLVPIRDKAGRIYKSYNAGENAFVDILQAESGRWIARATTVFQANQANESHDAPAAQPIMRVFKGDMLRIDHAGAEKFVKIVRLSPSNNLLYLVEHHQAGVFQTRHDDPEDSFRWLFASFDKLREWNAELVRIDTLGQPWRRKRGLETGSEDATRIGWTRPKKWP

SEQ ID NO: 110 - protein, 1358 aa, Acidaminococcus sp.
MGKMYYLGLDIGTNSVGYAVTDPSYHLLKFKGEPMWGAHVFAAGNQSAERRSFRTSRRRLDRRQQRVKLVQEIFAPVISPIDPRFFIRLHESALWRDDVAETDKHIFFNDPTYTDKEYYSDYPTIHHLIVDLMESSEKHDPRLVYLAVAWLVAHRGHFLNEVDKDNIGDVLSFDAFYPEFLAFLSDNGVSPWVCESKALQATLLSRNSVNDKYKALKSLIFGSQKPEDNFDANISEDGLIQLLAGKKVKVNKLFPQESNDASFTLNDKEDAIEEILGTLTPDECEWIAHIRRLFDWAIMKHALKDGRTISESKVKLYEQHHHDLTQLKYFVKTYLAKEYDDIFRNVDSETTKNYVAYSYHVKEVKGTLPKNKATQEEFCKYVLGKVKNIECSEADKVDFDEMIQRLTDNSFMPKQVSGENRVIPYQLYYYELKTILNKAASYLPFLTQCGKDAISNQDKLLSIMTFRIPYFVGPLRKDNSEHAWLERKAGKIYPWNFNDKVDLDKSEEAFIRRMTNTCTYYPGEDVLPLDSLIYEKFMILNEINNIRIDGYPISVDVKQQVFGLFEKKRRVTVKDIQNLLLSLGALDKHGKLTGIDTTIHSNYNTYHHFKSLMERGVLTRDDVERIVERMTYSDDTKRVRLWLNNNYGTLTADDVKHISRLRKHDFGRLSKMFLTGLKGVHKETGERASILDFMWNTNDNLMQLLSECYTFSDEITKLQEAYYAKAQLSLNDFLDSMYISNAVKRPIYRTLAVVNDIRKACGTAPKRIFIEMARDGESKKKRSVTRREQIKNLYRSIRKDFQQEVDFLEKILENKSDGQLQSDALYLYFAQLGRDMYTGDPIKLEHIKDQSFYNIDHIYPQSMVKDDSLDNKVLVQSEINGEKSSRYPLDAAIRNKMKPLWDAYYNHGLISLKKYQRLTRSTPFTDDEKWDFINRQLVETRQSTKALAILLKRKFPDTEIVYSKAGLSSDFRHEFGLVKSRNINDLHHAKDAFLAIVTGNVYHERFNRRWFMVNQPYSVKTKTLFTHSIKNGNFVAWNGEEDLGRIVKMLKQNKNTIHFTRFSFDRKEGLFDIQPLKASTGLVPRKAGLDVVKYGGYDKSTAAYYLLVRFTLEDKKTQHKLMMIPVEGLYKARIDHDKEFLTDYAQTTISEILQKDKQKVINIMFPMGTRHIKLNSMISIDGFYLSIGGKSSKGKSVLCHAMVPLIVPHKIECYIKAMESFARKFKENNKLRIVEKFDKITVEDNLNLYELFLQKLQHNPYNKFFSTQFDVLTNGRSTFTKLSPEEQVQTLLNILSIFKTCRSSGCDLKSINGSAQAARIMISADLTGLSKKYSDIRLVEQSASGLFVSKSQNLLEYL

SEQ ID NO: 111 - protein, 1082 aa, Methylosinus trichosporium
MRVLGLDAGIASLGWALIEIEESNRGELSQGTIIGAGTWMFDAPEEKTQAGAKLKSEQRRTFRGQRRVVRRRRQRMNEVRRILHSHGLLPSSDRDALKQPGLDPWRIRAEALDRLLGPVELAVALGHIARHRGFKSNSKGAKTNDPADDTSKMKRAVNETREKLARFGSAAKMLVEDESFVLRQTPTKNGASEIVRRFRNREGDYSRSLLRDDLAAEMRALFTAQARFQSAIATADLQTAFTKAAFFQRPLQDSEKLVGPCPFEVDEKRAPKRGYSFELFRFLSRLNHVTLRDGKQERTLTRDELALAAADFGAAAKVSFTALRKKLKLPETTVFVGVKADEESKLDVVARSGKAAEGTARLRSVIVDALGELAWGALLCSPEKLDKIAEVISFRSDIGRISEGLAQAGCNAPLVDALTAAASDGRFDPFTGAGHISSKAARNILSGLRQGMTYDKACCAADYDHTASRERGAFDVGGHGREALKRILQEERISRELVGSPTARKALIESIKQVKAIVERYGVPDRIHVELARDVGKSIEEREEITRGIEKRNRQKDKLRGLFEKEVGRPPQDGARGKEELLRFELWSEQMGRCLYTDDYISPSQLVATDDAVQVDHILPWSRFADDSYANKTLCMAKANQDKKGRTPYEWFKAEKTDTEWDAFIVRVEALADMKGFKKRNYKLRNAEEAAAKFRNRNLNDTRWACRLLAEALKQLYPKGEKDKDGKERRRVFSRPGALTDRLRRAWGLQWMKKSTKGDRIPDDRHHALDAIVIAATTESLLQRATREVQEIEDKGLHYDLVKNVTPPWPGFREQAVEAVEKVFVARAERRRARGKAHDATIRHIAVREGEQRVYERRKVAELKLADLDRVKDAERNARLIEKLRNWIEAGSPKDDPPLSPKGDPIFKVRLVTKSKVNIALDTGNPKRPGTVDRGEMARVDVFRKASKKGKYEYYLVPIYPHDIATMKTPPIRAVQAYKPEDEWPEMDSSYEFCWSLVPMTYLQVISSKGEIFEGYYRGMNRSVGAIQLSAHSNSSDVVQGIGARTLTEFKKFNVDRFGRKHEVERELRTWRGETWRGKAYI

SEQ ID NO: 112 - protein, 1105 aa, Actinomyces coleocanis
MDNKNYRIGIDVGLNSIGFCAVEVDQHDTPLGFLNLSVYRHDAGIDPNGKKTNTTRLAMSGVARRTRRLFRKRKRRLAALDRFIEAQGWTLPDHADYKDPYTPWLVRAELAQTPIRDENDLHEKLAIAVRHIARHRGWRSPWVPVRSLHVEQPPSDQYLALKERVEAKTLLQMPEGATPAEMVVALDLSVDVNLRPKNREKTDTRPENKKPGFLGGKLMQSDNANELRKIAKIQGLDDALLRELIELVFAADSPKGASGELVGYDVLPGQHGKRRAEKAHPAFQRYRIASIVSNLRIRHLGSGADERLDVETQKRVFEYLLNAKPTADITWSDVAEEIGVERNLLMGTATQTADGERASAKPPVDVTNVAFATCKIKPLKEWWLNADYEARCVMVSALSHAEKLTEGTAAEVEVAEFLQNLSDEDNEKLDSFSLPIGRAAYSVDSLERLTKRMIENGEDLFEARVNEFGVSEDWRPPAEPIGARVGNPAVDRVLKAVNRYLMAAEAEWGAPLSVNIEHVREGFISKRQAVEIDRENQKRYQRNQAVRSQIADHINATSGVRGSDVTRYLAIQRQNGECLYCGTAITFVNSEMDHIVPRAGLGSTNTRDNLVATCERCNKSKSNKPFAVWAAECGIPGVSVAEALKRVDFWIADGFASSKEHRELQKGVKDRLKRKVSDPEIDNRSMESVAWMARELAHRVQYYFDEKHTGTKVRVFRGSLTSAARKASGFESRVNFIGGNGKTRLDRRHHAMDAATVAMLRNSVAKTLVLRGNIRASERAIGAAETWKSFRGENVADRQIFESWSENMRVLVEKFNLALYNDEVSIFSSLRLQLGNGKAHDDTITKLQMHKVGDAWSLTEIDRASTPALWCALTRQPDFTWKDGLPANEDRTIIVNGTHYGPLDKVGIFGKAAASLLVRGGSVDIGSAIHHARIYRIAGKKPTYGMVRVFAPDLLRYRNEDLFNVELPPQSVSMRYAEPKVREAIREGKAEYLGWLVVGDELLLDLSSETSGQIAELQQDFPGTTHWTVAGFFSPSRLRLRPVYLAQEGLGEDVSEGSKSIIAGQGWRPAVNKVFGSAMPEVIRRDGLGRKRRFSYSGLPVSWQG

SEQ ID NO: 113 - protein, 1442 aa, unidentified
MPVLSPLSPNAAQGRRRWSLALDIGEGSIGWAVAEVDAEGRVLQLTGTGVTLFPSAWSNENGTYVAHGAADRAVRGQQQRHDSRRRRLAGLARLCAPVLERSPEDLKDLTRTPPKADPRAIFFLRADAARRPLDGPELFRVLHHMAAHRGIRLAELQEVDPPPESDADDAAPAATEDEDGTRRAAADERAFRRLMAEHMHRHGTQPTCGEIMAGRLRETPAGAQPVTRARDGLRVGGGVAVPTRALIEQEFDAIRAIQAPRHPDLPWDSLRRLVLDQAPIAVPPATPCLFLEELRRRGETFQGRTITREAIDRGLTVDPLIQALRIRETVGNLRLHERITEPDGRQRYVPRAMPELGLSHGELTAPERDTLVRALMHDPDGLAAKDGRIPYTRLRKLIGYDNSPVCFAQERDTSGGGITVNPTDPLMARWIDGWVDLPLKARSLYVRDVVARGADSAALARLLAEGAHGVPPVAAAAVPAATAAILESDIMQPGRYSVCPWAAEAILDAWANAPTEGFYDVTRGLFGFAPGEIVLEDLRRARGALLAHLPRTMAAARTPNRAAQQRGPLPAYESVIPSQLITSLRRAHKGRAADWSAADPEERNPFLRTWTGNAATDHILNQVRKTANEVITKYGNRRGWDPLPSRITVELAREAKHGVIRRNEIAKENRENEGRRKKESAALDTFCQDNTVSWQAGGLPKERAALRLRLAQRQEFFCPYCAERPKLRATDLFSPAETEIDHVIERRMGGDGPDNLVLAHKDCNNAKGKKTPHEHAGDLLDSPALAALWQGWRKENADRLKGKGHKARTPREDKDFMDRVGWRFEEDARAKAEENQERRGRRMLHDTARATRLARLYLAAAVMPEDPAEIGAPPVETPPSPEDPTGYTAIYRTISRVQPVNGSVTHMLRQRLLQRDKNRDYQTHHAEDACLLLLAGPAVVQAFNTEAAQHGADAPDDRPVDLMPTSDAYHQQRRARALGRVPLATVDAALADIVMPESDRQDPETGRVHWRLTRAGRGLKRRIDDLTRNCVILSRPRRPSETGTPGALHNATHYGRREITVDGRTDTVVTQRMNARDLVALLDNAKIVPAARLDAAAPGDTILKEICTEIADRHDRVVDPEGTHARRWISARLAALVPAHAEAVARDIAELADLDALADADRTPEQEARRSALRQSPYLGRAISAKKADGRARAREQEILTRALLDPHWGPRGLRHLIMREARAPSLVRIRANKTDAFGRPVPDAAVWVKTDGNAVSQLWRLTSVVTDDGRRIPLPKPIEKRIEISNLEYARLNGLDEGAGVTGNNAPPRPLRQDIDRLTPLWRDHGTAPGGYLGTAVGELEDKARSALRGKAMRQTLTDAGITAEAGWRLDSEGAVCDLEVAKGDTVKKDGKTYKVGVITQGIFGMPVDAAGSAPRTPEDCEKFEEQYGIKPWKAKGIPLA

SEQ ID NO: 114 - protein, 1384 aa, Coriobacterium glomerans
MKLRGIEDDYSIGLDMGTSSVGWAVTDERGTLAHFKRKPTWGSRLFREAQTAAVARMPRGQRRRYVRRRWRLDLLQKLFEQQMEQADPDFFIRLRQSRLLRDDRAEEHADYRWPLFNDCKFTERDYYQRFPTIYHVRSWLMETDEQADIRLIYLALHNIVKHRGNFLREGQSLSAKSARPDEALNHLRETLRVWSSERGFECSIADNGSILAMLTHPDLSPSDRRKKIAPLFDVKSDDAAADKKLGIALAGAVIGLKTEFKNIFGDFPCEDSSIYLSNDEAVDAVRSACPDDCAELFDRLCEVYSAYVLQGLLSYAPGQTISANMVEKYRRYGEDLALLKKLVKIYAPDQYRMFFSGATYPGTGIYDAAQARGYTKYNLGPKKSEYKPSESMQYDDFRKAVEKLFAKTDARADERYRMMMDRFDKQQFLRRLKTSDNGSIYHQLHLEELKAIVENQGRFYPFLKRDADKLVSLVSFRIPYYVGPLSTRNARTDQHGENRFAWSERKPGMQDEPIFPWNWESIIDRSKSAEKFILRMTGMCTYLQQEPVLPKSSLLYEEFCVLNELNGAHWSIDGDDEHRFDAADREGIIEELFRRKRTVSYGDVAGWMERERNQIGAHVCGGQGEKGFESKLGSYIFFCKDVFKVERLEQSDYPMIERIILWNTLFEDRKILSQRLKEEYGSRLSAEQIKTICKKRFTGWGRLSEKFLTGITVQVDEDSVSIMDVLREGCPVSGKRGRAMVMMEILRDEELGFQKKVDDFNRAFFAENAQALGVNELPGSPAVRRSLNQSIRIVDEIASIAGKAPANIFIEVTRDEDPKKKGRRTKRRYNDLKDALEAFKKEDPELWRELCETAPNDMDERLSLYFMQRGKCLYSGRAIDIHQLSNAGIYEVDHIIPRTYVKDDSLENKALVYREENQRKTDMLLIDPEIRRRMSGYWRMLHEAKLIGDKKFRNLLRSRIDDKALKGFIARQLVETGQMVKLVRSLLEARYPETNIISVKASISHDLRTAAELVKCREANDFHHAHDAFLACRVGLFIQKRHPCVYENPIGLSQVVRNYVRQQADIFKRCRTIPGSSGFIVNSFMTSGFDKETGEIFKDDWDAEAEVEGIRRSLNFRQCFISRMPFEDHGVFWDATIYSPRAKKTAALPLKQGLNPSRYGSFSREQFAYFFIYKARNPRKEQTLFEFAQVPVRLSAQIRQDENALERYARELAKDQGLEFIRIERSKILKNQLIEIDGDRLCITGKEEVRNACELAFAQDEMRVIRMLVSEKPVSRECVISLFNRILLHGDQASRRLSKQLKLALLSEAFSEASDNVQRNVVLGLIAIFNGSTNMVNLSDIGGSKFAGNVRIKYKKELASPKVNVHLIDQSVTGMFERRTKIGL

SEQ ID NO: 115 - protein, 4 aa, synthetic construct
AGGG

SEQ ID NO: 116 - protein, 1477 aa, synthetic construct
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEDKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAIMKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTEIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDCWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKK

SEQ ID NO: 117 - protein, 1403 aa, synthetic construct
MLFFMSTDITNKPREKGVFDNFTNLYEFSKTLTFGLIPLKWDDNKKMIVEDEDFSVLRKYGVIEEDKRIAESIKIAKFYLNILHRELIGKVLGSLKFEKKNLENYDRLLGEIEKNNKNENISEDKKKEIRKNFKKELSIAQDILLKKVGEVFESNGSGILSSKNCLDELTKRFTRQEVDKLRRENKDIGVEYPDVAYREKDGKEETKSFFAMDVGYLDDFHKNRKQLYSVKGKKNSLGRRILDNFEIFCKNKKLYEKYKNLDIDFSEIERNFNLTLEKVFDFDNYNERLTQEGLDEYAKILGGESNKQERTANIHGLNQIINLYIQKKQSEQKAEQKETGKKKIKFNKKDYPTFTCLQKQILSQVFRKEIIIESDRDLIRELKFFVEESKEKVDKARGIIEFLLNHEENDIDLAMVYLPKSKINSFVYKVFKEPQDFLSVFQDGASNLDFVSFDKIKTHLENNKLTYKIFFKTLIKENHDFESFLILLQQEIDLLIDGGETVTLGGKKESITSLDEKKNRLKEKLGWFEGKVRENEKMKDEEEGEFCSTVLAYSQAVLNITKRAEIFWLNEKQDAKVGEDNKDMIFYKKFDEFADDGFAPFFYFDKFGNYLKRRSRNTTKEIKLHFGNDDLLEGWDMNKEPEYWSFILRDRNQYYLGIGKKDGEIFHKKLGNSVEAVKEAYELENEADFYEKIDYKQLNIDRFEGIAFPKKTKTEEAFRQVCKKRADEFLGGDTYEFKILLAIKKEYDDFKARRQKEKDWDSKFSKEKMSKLIEYYITCLGKRDDWKRFNLNFRQPKEYEDRSDFVRHIQRQAYWIDPRKVSKDYVDKKVAEGEMFLFKVHNKDFYDFERKSEDKKNHTANLFTQYLLELFSCENIKNIKSKDLIESIFELDGKAEIRFRPKTDDVKLKIYQKKGKDVTYADKRDGNKEKEVIQHRRFAKDALTLHLKIRLNFGKHVNLFDFNKLVNTELFAKVPVKILGMDRGENNLIYYCFLDEHGEIENGKCGSLNRVGEQIITLEDDKKVKEPVDYFQLLVDREGQRDWEQKNWQKMTRIKDLKKAYLGNVVSWISKEMLSGIKEGVVTIGVLEDLNSNFKRTRFFRERQVYQGFEKALVNKLGYLVDKKYDNYRNVYQFAPIVDSVEEMEKNKQIGTLVYVPASYTSKICPHPKCGWRERLYMKNSASKEKIVGLLKSDGIKISYDQKNDRFYFEYQWEQEHKSDGKKKKYSGVDKVFSNVSRMRWDVEQKKSIDFVDGTDGSITNKLKSLLKGKGIELDNINQQIVNQQKELGVEFFQSIIFYFNLIMQIRNYDKEKSGSEADYIQCPSCLFDSRKPEMNGKLSAITNGDANGAYNIARKGFMQLCRIRENPQEPMKLITNREWDEAVREWDIYSAAQKIPVLSEEN

SEQ ID NO: 118 - protein, 1373 aa, synthetic construct
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 119 - protein, 1352 aa, synthetic construct
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITFSKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKEKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 120 - protein, 1334 aa, synthetic construct
MSSLTKFTNKYSKQLTIKNELIPVGKTLENIKENGLIDGDEQLNENYQKAKIIVDDFLRDFINKALNNTQIGNWRELADALNKEDEDNIEKLQDKIRGIIVSKFETFDLFSSYSIKKDEKIIDDDNDVEEEELDLGKKTSSFKYIFKKNLFKLVLPSYLKTTNQDKLKIISSFDNFSTYFRGFFENRKNIFTKKPISTSIAYRIVHDNFPKFLDNIRCFNVWQTECPQLIVKADNYLKSKNVIAKDKSLANYFTVGAYDYFLSQNGIDFYNNIIGGLPAFAGHEKIQGLNEFINQECQKDSELKSKLKNRHAFKMAVLFKQILSDREKSFVIDEFESDAQVIDAVKNFYAEQCKDNNVIFNLLNLIKNIAFLSDDELDGIFIEGKYLSSVSQKLYSDWSKLRNDIEDSANSKQGNKELAKKIKTNKGDVEKAISKYEFSLSELNSIVHDNTKFSDLLSCTLHKVASEKLVKVNEGDWPKHLKNNEEKQKIKEPLDALLEIYNTLLIFNCKSFNKNGNFYVDYDRCINELSSVVYLYNKTRNYCTKKPYNTDKFKLNFNSPQLGEGFSKSKENDCLTLLFKKDDNYYVGIIRKGAKINFDDTQAIADNTDNCIFKMNYFLLKDAKKFIPKCSIQLKEVKAHFKKSEDDYILSDKEKFASPLVIKKSTFLLATAHVKGKKGNIKKFQKEYSKENPTEYRNSLNEWIAFCKEFLKTYKAATIFDITTLKKAEEYADIVEFYKDVDNLCYKLEFCPIKTSFIENLIDNGDLYLFRINNKDFSSKSTGTKNLHTLYLQAIFDERNLNNPTIMLNGGAELFYRKESIEQKNRITHKAGSILVNKVCKDGTSLDDKIRNEIYQYENKFIDTLSDEAKKVLPNVIKKEATHDITKDKRFTSDKFFFHCPLTINYKEGDTKQFNNEVLSFLRGNPDINIIGIDRGERNLIYVTVINQKGEILDSVSFNTVTNKSSKIEQTVDYEEKLAVREKERIEAKRSWDSISKIATLKEGYLSAIVHEICLLMIKHNAIVVLENLNAGFKRIRGGLSEKSVYQKFEKMLINKLNYFVSKKESDWNKPSGLLNGLQLSDQFESFEKLGIQSGFIFYVPAAYTSKIDPTTGFANVLNLSKVRNVDAIKSFFSNFNEISYSKKEALFKFSFDLDSLSKKGFSSFVKFSKSKWNVYTFGERIIKPKNKQGYREDKRINLTFEMKKLLNEYKVSFDLENNLIPNLTSANLKDTFWKELFFIFKTTLQLRNSVTNGKEDVLISPVKNAKGEFFVSGTHNKTLPQDCDANGAYHIALKGLMILERNNLVREEKDTKKIMAISNVDWFEYVQKRRGVL

SEQ ID NO: 121 - protein, 1331 aa, synthetic construct
MKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITFSKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKEKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 122 - protein, 1326 aa, synthetic construct
MKVMENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNDKKLNLSISSTEWLDFVREKPYLK

SEQ ID NO: 123 - protein, 1318 aa, synthetic construct
MTNKFTNQYSLSKTLRFELIPQGKTLEFIQEKGLLSQDKQRAESYQEMKKTIDKFHKYFIDLALSNAKLTHLETYLELYNKSAETKKEQKFKDDLKKVQDNLRKEIVKSFSDGDAKSIFAILDKKELITVELEKWFENNEQKDIYFDEKFKTFTTYFTGFHQNRKNMYSVEPNSTAIAYRLIHENLPKFLENAKAFEKIKQVESLQVNFRELMGEFGDEGLIFVNELEEMFQINYYNDVLSQNGITIYNSIISGFTKNDIKYKGLNEYINNYNQTKDKKDRLPKLKQLYKQILSDRISLSFLPDAFTDGKQVLKAIFDFYKINLLSYTIEGQEESQNLLLLIRQTIENLSSFDTQKIYLKNDTHLTTISQQVFGDFSVFSTALNYWYETKVNPKFETEYSKANEKKREILDKAKAVFTKQDYFSIAFLQEVLSEYILTLDHTSDIVKKHSSNCIADYFKNHFVAKKENETDKTFDFIANITAKYQCIQGILENADQYEDELKQDQKLIDNLKFFLDAILELLHFIKPLHLKSESITEKDTAFYDVFENYYEALSLLTPLYNMVRNYVTQKPYSTEKIKLNFENAQLLNGWDANKEGDYLTTILKKDGNYFLAIMDKKHNKAFQKFPEGKENYEKMVYKLLPGVNKMLPKVFFSNKNIAYFNPSKELLENYKKETHKKGDTFNLEHCHTLIDFFKDSLNKHEDWKYFDFQFSETKSYQDLSGFYREVEHQGYKINFKNIDSEYIDGLVNEGKLFLFQIYSKDFSPFSKGKPNMHTLYWKALFEEQNLQNVIYKLNGQAEIFFRKASIKPKNIILHKKKIKIAKKHFIDKKTKTSEIVPVQTIKNLNMYYQGKISEKELTQDDLRYIDNFSIFNEKNKTIDIIKDKRFTVDKFQFHVPITMNFKATGGSYINQTVLEYLQNNPEVKIIGLDRGERHLVYLTLIDQQGNILKQESLNTITDSKISTPYHKLLDNKENERDLARKNWGTVENIKELKEGYISQVVHKIATLMLEENAIVVMEDLNFGFKRGRFKVEKQIYQKLEKMLIDKLNYLVLKDKQPQELGGLYNALQLTNKFESFQKMGKQSGFLFYVPAWNTSKIDPTTGFVNYFYTKYENVDKAKAFFEKFEAIRFNAEKKYFEFEVKKYSDFNPKAEGTQQAWTICTYGERIETKRQKDQNNKFVSTPINLTEKIEDFLGKNQIVYGDGNCIKSQIASKDDKAFFETLLYWFKMTLQMRNSETRTDIDYLISPVMNDNGTFYNSRDYEKLENPTLPKDADANGAYHIAKKGLMLLNKIDQADLTKKVDLSISNRDWLQFVQKNK

SEQ ID NO: 124 - protein, 1310 aa, synthetic construct
MFEKLSNIVSISKTIRFKLIPVGKTLENIEKLGKLEKDFERSDFYPILKNISDDYYRQYIKEKLSDLNLDWQKLYDAHELLDSSKKESQKNLEMIQAQYRKVLFNILSGELDKSGEKNSKDLIKNNKALYGKLFKKQFILEVLPDFVNNNDSYSEEDLEGLNLYSKFTTRLKNFWETRKNVFTDKDIVTAIPFRAVNENFGFYYDNIKIFNKNIEYLENKIPNLENELKEADILDDNRSVKDYFTPNGFNYVITQDGIDVYQAIRGGFTKENGEKVQGINEILNLTQQQLRRKPETKNVKLGVLTKLRKQILEYSESTSFLIDQIEDDNDLVDRINKFNVSFFESTEVSPSLFEQIERLYNALKSIKKEEVYIDARNTQKFSQMLFGQWDVIRRGYTVKITEGSKEEKKKYKEYLELDETSKAKRYLNIREIEELVNLVEGFEEVDVFSVLLEKFKMNNIERSEFEAPIYGSPIKLEAIKEYLEKHLEEYHKWKLLLIGNDDLDTDETFYPLLNEVISDYYIIPLYNLTRNYLTRKHSDKDKIKVNFDFPTLADGWSESKISDNRSIILRKGGYYYLGILIDNKLLINKKNKSKKIYEILIYNQIPEFSKSIPNYPFTKKVKEHFKNNVSDFQLIDGYVSPLIITKEIYDIKKEKKYKKDFYKDNNTNKNYLYTIYKWIEFCKQFLYKYKGPNKESYKEMYDFSTLKDTSLYVNLNDFYADVNSCAYRVLFNKIDENTIDNAVEDGKLLLFQIYNKDFSPESKGKKNLHTLYWLSMFSEENLRTRKLKLNGQAEIFYRKKLEKKPIIHKEGSILLNKIDKEGNTIPENIYHECYRYLNKKIGREDLSDEAIALFNKDVLKYKEARFDIIKDRRYSESQFFFHVPITFNWDIKTNKNVNQIVQGMIKDGEIKHIIGIDRGERHLLYYSVIDLEGNIVEQGSLNTLEQNRFDNSTVKVDYQNKLRTREEDRDRARKNWTNINKIKELKDGYLSHVVHKLSRLIIKYEAIVIMENLNQGFKRGRFKVERQVYQKFELALMNKLSALSFKEKYDERKNLEPSGILNPIQACYPVDAYQELQGQNGIVFYLPAAYTSVIDPVTGFTNLFRLKSINSSKYEEFIKKFKNIYFDNEEEDFKFIFNYKDFAKANLVILNNIKSKDWKISTRGERISYNSKKKEYFYVQPTEFLINKLKELNIDYENIDIIPLIDNLEEKAKRKILKALFDTFKYSVQLRNYDFENDYIISPTADDNGNYYNSNEIDIDKTNLPNNGDANGAFNIARKGLLLKDRIVNSNESKVDLKIKNEDWINFIIS

SEQ ID NO: 125 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 126 - protein, 1307 aa, synthetic construct
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISKYINDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQVAPKNLDNPSKKEQDLIAKKTEKAKYLSLETIKLALEEFNKHRDIDKQCRFEEILSNFAAIPMIFDEIAQNKDNLAQISIKYQNQGKKDLLQASAEEDVKAIKDLLDQTNNLLHRLKIFHISQSEDKANILDKDEHFYLVFEECYFELANIVPLYNKIRNYITQKPYSDEKFKLNFENSTLASGWDKNKESANTAILFIKDDKYYLGIMDKKHNKIFSDKAIEENKGEGYKKIVYKQIADASKDIQNLMIIDGKTVCKKGRKDRNGVNRQLLSLKRKHLPENIYRIKETKSYLKNEARFSRKDLYDFIDYYKDRLDYYDFEFELKPSNEYSDFNDFTNHIGSQGYKLTFENISQDYINSLVNEGKLYLFQIYSKDFSAYSKGRPNLHTLYWKALFDERNLQDVVYKLNGEAELFYRKQSIPKKITHPAKETIANKNKDNPKKESVFEYDLIKDKRFTEDKFFFHCPITINFKSSGANKFNDEINLLLKEKANDVHILSIDRGERHLAYYTLVDGKGNIIKQDNFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 127 - protein, 1305 aa, Eubacterium sp.
MNKAADNYTGGNYDEFIALSKVQKTLRNELKPTPFTAEHIKQRGIISEDEYRAQQSLELKKIADEYYRNYITHKLNDINNLDFYNLFDAIEEKYKKNDKDNRDKLDLVEKSKRGEIAKMLSADDNFKSMFEAKLITKLLPDYVERNYTGEDKEKALETLALFKGFTTYFKGYFKTRKNMFSGEGGASSICHRIVNVNASIFYDNLKTFMRIQEKAGDEIALIEEELTEKLDGWRLEHIFSRDYYNEVLAQKGIDYYNQICGDINKHMNLYCQQNKFKANIFKMMKIQKQIMGISEKAFEIPPMYQNDEEVYASFNEFISRLEEVKLTDRLINILQNINIYNTAKIYINARYYTNVSSYVYGGWGVIDSAIERYLYNTIAGKGQSKVKKIENAKKDNKFMSVKELDSIVAEYEPDYFNAPYIDDDDNAVKAFGGQGVLGYFNKMSELLADVSLYTIDYNSDDSLIENKESALRIKKQLDDIMSLYHWLQTFIIDEVVEKDNAFYAELEDICCELENVVTLYDRIRNYVTKKPYSTQKFKLNFASPTLAAGWSRSKEFDNNAIILLRNNKYYIAIFNVNNKPDKQIIKGSEEQRLSTDYKKMVYNLLPGPNKMLPKVFIKSDTGKRDYNPSSYILEGYEKNRHIKSSGNFDINYCHDLIDYYKACINKHPEWKNYGFKFKETNQYNDIGQFYKDVEKQGYSISWAYISEEDINKLDEEGKIYLFEIYNKDLSAHSTGRDNLHTMYLKNIFSEDNLKNICIELNGEAELFYRKSSMKSNITHKKDTILVNKTYINETGVRVSLSDEDYMKVYNYYNNNYVIDTENDKNLIDIIEKIGHRKSKIDIVKDKRYTEDKYFLYLPITINYGIEDENVNSKIIEYIAKQDNMNVIGIDRGERNLIYISVIDNKGNIIEQKSFNLVNNYDYKNKLKNMEKTRDNARKNWQEIGKIKDVKSGYLSGVISKIARMVIDYNAIIVMEDLNKGFKRGRFKVERQVYQKFENMLISKLNYLVFKERKADENGGILRGYQLTYIPKSIKNVGKQCGCIFYVPAAYTSKIDPATGFINIFDFKKYSGSGINAKVKDKKEFLMSMNSIRYINECSEEYEKIGHRELFAFSFDYNNFKTYNVSSPVNEWTAYTYGERIKKLYKDGRWLRSEVLNLTENLIKLMEQYNIEYKDGHDIREDISHMDETRNADFICSLFEELKYTVQLRNSKSEAEDENYDRLVSPILNSSNGFYDSSDYMENENNTTHTMPKDADANGAYCIALKGLYEINKIKQNWSDDKKFKENELYINVTEWLDYIQNRRFE

SEQ ID NO: 128 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQVAPKNLDNPSKKEQDLIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGNPQKGYEKFEFNIEDCRKFIDFYKESISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKKSIPKKITHPAKEAIANKNKDNPKKESFFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEHNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSILNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 129 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSDDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQVAPKNLDNPSKKEQDLIAKKTEKAKYLSLETIKLALEEFNKHRDIDKQCRFEEILANFAAIPMIFDEIAQNKDNLAQISLKYQNQGKKDLLQASAEEDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGNPQKGYEKFEFNIEDCRKFIDFYKESISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEHNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 130 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 131 - protein, 1285 aa, unidentified
MKSFDSFTNLYSLSKTLKFEMRPVGNTQKMLDNAGVFEKDKLIQKKYGKTKPYFDRLHREFIEEALTGVELIGLDENFRTLVDWQKDKKNNVAMKAYENSLQRLRTEIGKIFNLKAEDWVKNKYPILGLKNKNTDILFEEAVFGILKARYGEEKDTFIEVEEIDKTGKSKINQISIFDSWKGFTGYFKKFFETRKNFYKNDGTSTAIATRIIDQNLKRFIDNLSIVESVRQKVDLAETEKSFSISLSQFFSIDFYNKCLLQDGIDYYNKIIGGETLKNGEKLIGLNELINQYRQNNKDQKIPFFKLLDKQILSEKILFLDEIKNDTELIEALSQFAKTAEEKTKIVKKLFADFVENNSKYDLAQIYISQEAFNTISNKWTSETETFAKYLFEAMKSGKLAKYEKKDNSYKFPDFIALSQMKSALLSISLEGHFWKEKYYKISKFQEKTNWEQFLAIFLYEFNSLFSDKINTKDGETKQVGYYLFAKDLHNLILSEQIDIPKDSKVTIKDFADSVLTIYQMAKYFAVEKKRAWLAEYELDSFYTQPDTGYLQFYDNAYEDIVQVYNKLRNYLTKKPYSEEKWKLNFENSTLANGWDKNKESDNSAVILQKGGKYYLGLITKGHNKIFDDRFQEKFIVGIEGGKYEKIVYKFFPDQAKMFPKVCFSAKGLEFFRPSEEILRIYNNAEFKKGETYSIDSMQKLIDFYKDCLTKYEGWACYTFRHLKPTEEYQNNIGEFFRDVAEDGYRIDFQGISDQYIHEKNEKGELHLFEIHNKDWNLDKARDGKSKTTQKNLHTLYFESLFSNDNVVQNFPIKLNGQAEIFYRPKTEKDKLESKKDKKGNKVIDHKRYSENKIFFHVPLTLNRTKNDSYRFNAQINNFLANNKDINIIGVDRGEKHLVYYSVITQASDILESGSLNELNGVNYAEKLGKKAENREQARRDWQDVQGIKDLKKGYISQVVRKLADLAIKHNAIIILEDLNMRFKQVRGGIEKSIYQQLEKALIDKLSFLVDKGEKNPEQAGHLLKAYQLSAPFETFQKMGKQTGIIFYTQASYTSKSDPVTGWRPHLYLKYFSAKKAKDDIAKFTKIEFVNDRFELTYDIKDFQQAKEYPNKTVWKVCSNVERFRWDKNLNQNKGGYTHYTNITENIQELFTKYGIDITKDLLTQISTIDEKQNTSFFRDFIFYFNLICQIRNTDDSEIAKKNGKDDFILSPVEPFFDSRKDNGNKLPENGDDNGAYNIARKGIVILNKISQYSEKNENCEKMKWGDLYVSNIDWDNFVTQANARH

SEQ ID NO: 132 - protein, 1282 aa, Eubacterium eligens
MNGNRSIVYREFVGVTPVAKTLRNELRPVGHTQEHIIQNGLIQEDELRQEKSTELKNIMDDYYREYIDKSLSGLTDLDFTLLFELMNSVQSSLSKDNKKALEKEHNKMREQICTHLQSDSDYKNMFNAKLFKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDEHISLIESEEKADEIKKRLDMYMNMYHWVKAFIVDEVLDRDEMFYSDIDDIYNILENIVPLYNRVRNYVTQKPYTSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYNDISEFYREVEMQGYRIDWTYISEADINKLDEEGKIYLFQIYNKDFAENSTGKENLHTMYFKNIFSEENLKNIVIKLNGQAELFYRKASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMAVKYIAQNDDIHVIGIDRGERNLIYISVIDSHGNIVKQKSYNILNNYDYKKKLVEKEKTREYARKNWKSIGNIKELKEGYISGVVHEIAMLMVEYNAIIAMEDLNYGFKRGRFKVERQVYQKFESMLINKLNYFASKGKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDVRIDMEKMYEDKNSEFFAQLLSLYKLTVQMRNSYTEAEEQEKGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 133 - protein, 508 aa, Eubacterium ramulus
MIKKTIDTVLNVRPIFVGIQHLYFYEGPCRFGEGDELMPEYDAMMNQEMNAAYVNEVVQHETEGVHIMDPIYVERDDWFRSPEAMYEKMAEDIDKVDFYLFHFGIGRGDIYLEFAERYKKPVGAAPGLCCDGIGNTAAVKNRGLEAYAFMSWDEFDTWMRVLRVRKCLKNTRVLLAVRWDSNRSYSSYDNFINQSDVTNKWGIQFRHVNVHELLDQTHPVDPTTNPSTPGRKALNINDEDMKEIEKITDELIANAEACTMEPDMVKKTIQAYYTVQKLLDAYDCNAFTAPCPDLCSTRRFSEEKFTLCMTHSLNDENGISSACEYDINSVIGKVIMTNLSGKAPYMGNTNAIVFDKEGHMIPFHKFNDNTIEDIADKTNLYMTFHSTPNRNLKGLKAEKERYRLAPFAYSGFGATIRYDFAQDIGQVITMIRISPDATKIFIAKGTISGGAGYEMKNCDQGVFFNVADKVDFYHKQQYFGNHTVLAYGDYVEELKMLAEALGIEAVIA

SEQ ID NO: 134 - protein, 1273 aa, Flavobacterium sp.
MKNFSNLYQVSKTVRFELKPIGNTLENIKNKSLLKNDSIRAESYQKMKKTIDEFHKYFIDLALNNKKLSYLNEYIALYTQSAEAKKEDKFKADFKKVQDNLRKEIVSSFTEGEAKAIFSVLDKKELITIELEKWKNENNLAVYLDESFKSFTTYFTGFHQNRKNMYSAEANSTAIAYRLIHENLPKFIENSKAFEKSSQIAELQPKIEKLYKEFEAYLNVNSISELFEIDYFNEVLTQKGITVYNNIIGGRTATEGKQKIQGLNEIINLYNQTKPKNERLPKLKQLYKQILSDRISLSFLPDAFTEGKQVLKAVFEFYKINLLSYKQDGVEESQNLLELIQQVVKNLGNQDVNKIYLKNDTSLTTIAQQLFGDFSVFSAALQYRYETVVNPKYTAEYQKANEAKQEKLDKEKIKFVKQDYFSIAFLQEVVADYVKTLDENLDWKQKYTPSCIADYFTTHFIAKKENEADKTFNFIANIKAKYQCIQGILEQADDYEDELKQDQKLIDNIKFFLDAILEVVHFIKPLHLKSESITEKDNAFYDVFENYYEALNVVTPLYNMVRNYVTQKPYSTEKIKLNFENAQLLNGWDANKEKDYLTTILKRDGNYFLAIMDKKHNKTFQQFTEDDENYEKIVYKLLPGVNKMLPKVFFSNKNIAFFNPSKEILDNYKNNTHKKGATFNLKDCHALIDFFKDSLNKHEDWKYFDFQFSETKTYQDLSGFYKEVEHQGYKINFKKVSVSQIDTLIEEGKMYLFQIYNKDFSPYAKGKPNMHTLYWKALFETQNLENVIYKLNGQAEIFFRKASIKKKNIITHKAHQPIAAKNPLTPTAKNTFAYDLIKDKRYTVDKFQFHVPITMNFKATGNSYINQDVLAYLKDNPEVNIIGLDRGERHLVYLTLIDQKGTILLQESLNVIQDEKTHTPYHTLLDNKEIARDKARKNWGSIESIKELKEGYISQVVHKITKMMIEHNAIVVMEDLNFGFKRGRFKVEKQIYQKLEKMLIDKLNYLVLKDKQPHELGGLYNALQLTNKFESFQKMGKQSGFLFYVPAWNTSKIDPTTGFVNYFYTKYENVEKAKTFFSKFDSILYNKTKGYFEFVVKNYSDFNPKAADTRQEWTICTHGERIETKRQKEQNNNFVSTTIQLTEQFVNFFEKVGLDLSKELKTQLIAQNEKSFFEELFHLLKLTLQMRNSESHTEIDYLISPVANEKGIFYDSRKATASLPIDADANGAYHIAKKGLWIMEQINKTNSEDDLKKVKLAISNREWLQYVQQVQKK

SEQ ID NO: 135 - protein, 1264 aa, Prevotella brevis
MKQFTNLYQLSKTLRFELKPIGKTLEHINANGFIDNDAHRAESYKKVKKLIDDYHKDYIENVLNNFKLNGEYLQAYFDLYSQDTKDKQFKDIQDKLRKSIASALKGDDRYKTIDKKELIRQDMKTFLKKDTDKALLDEFYEFTTYFTGYHENRKNMYSDEAKSTAIAYRLIHDNLPKFIDNIAVFKKIANTSVADNFSTIYKNFEEYLNVNSIDEIFSLDYYNIVLTQTQIEVYNSIIGGRTLEDDTKIQGINEFVNLYNQQLANKKDRLPKLKPLFKQILSDRVQLSWLQEEFNTGADVLNAVKEYCTSYFDNVEESVKVLLTGISDYDLSKIYITNDLALTDVSQRMFGEWSIIPNAIEQRLRSDNPKKTNEKEEKYSDRISKLKKLPKSYSLGYINECISELNGIDIADYYATLGAINTESKQEPSIPTSIQVHYNALKPILDTDYPREKNLSQDKLTVMQLKDLLDDFKALQHFIKPLLGNGDEAEKDEKFYGELMQLWEVIDSITPLYNKVRNYCTRKPFSTEKIKVNFENAQLLDGWDENKESTNASIILRKNGMYYLGIMKKEYRNILTKPMPSDGDCYDKVVYKFFKDITTMVPKCTTQMKSVKEHFSNSNDDYTLFEKDKFIAPVVITKEIFDLNNVLYNGVKKFQIGYLNNTGDSFGYNHAVEIWKSFCLKFLKAYKSTSIYDFSSIEKNIGCYNDLNSFYGAVNLLLYNLTYRKVSVDYIHQLVDEDKMYLFMIYNKDFSTYSKGTPNMHTLYWKMLFDESNLNDVVYKLNGQAEVFYRKKSITYQHPTHPANKPIDNKNVNNPKKQSNFEYDLIKDKRYTVDKFMFHVPITLNFKGMGNGDINMQVREYIKTTDDLHFIGIDRGERHLLYICVINGKGEIVEQYSLNEIVNNYKGTEYKTDYHTLLSERDKKRKEERSSWQTIEGIKELKSGYLSQVIHKITQLMIKYNAIVLLEDLNMGFKRGRQKVESSVYQQFEKALIDKLNYLVDKNKDANEIGGLLHAYQLTNDPKLPNKNSKQSGFLFYVPAWNTSKIDPVTGFVNLLDTRYENVAKAQAFFKKFDSIRYNKEYDRFEFKFDYSNFTAKAEDTRTQWTLCTYGTRIETFRNAEKNSNWDSREIDLTTEWKTLFTQHNIPLNANLKEAILLQANKNFYTDILHLMKLTLQMRNSVTGTDIDYMVSPVANECGEFFDSRKVKEGLPVNADANGAYNIARKGLWLAQQIKNANDLSDVKLAITNKEWLQFAQKKQYLKD

SEQ ID NO: 136 - protein, 1264 aa, Moraxella caprae
MLFQDFTHLYPLSKTMRFELKPIGKTLEHIHAKNFLSQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAITYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGALYDELAKIPTLYNKVRDYLSQKPFSTEKYKLNFGNPTLLNGWDLNKEKDNFGIILQKDGCYYLALLDKAHKKVFDNAPNTGKNVYQKMIYKLLPGPNKMLPKVFFAKSNLDYYNPSAELLDKYAQGTHKKGNNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSKDNLANPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDEADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWKICSHGDKRYVYDKTANQNKGATKGINVNDELKSLFARHHINDKQPNLVMDICQNNDKEFHKSLIYLLKTLLALRYSNASSDEDFILSPVANDEGMFFNSALADDTQPQNADANGAYHIALKGLWVLEQIKNSDDLNKVKLAIDNQTWLNFAQNR

SEQ ID NO: 137 - protein, 1263 aa, Leptospira inadai
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNLPNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDSIGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 138 - protein, 1262 aa, Porphyromonas crevioricanis
MPWIDLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKVLPEYKEGEPYFEKMDYKFLPDPNKMLPKVFLSKKGIEIYEPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQERRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHAQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 139 - protein, 1262 aa, unidentified
MRKFNEFVGLYPISKTLRFELKPIGKTLEHIQRNKLLEHDAVRADDYVKVKKIIDKYHKCLIDEALSGFTFDTEADGRSNNSLSEYYLYYNLKKRNEQEQKTFKTIQNNLRKQIVNKLTQSEKYKRIDKKELITTDLPDFLTNESEKELVEKFKNFTTYFTEFHKNRKNMYSKEEKSTAIAFRLINENLPKFVDNIAAFEKVVSSPLAEKINALYEDFKEYLNVEEISRVFRLDYYDELLTQKQIDLYNAIVGGRTEEDNKIQIKGLNQYINEYNQQQTDRSNRLPKLKPLYKQILSDRESVSWLPPKFDSDKNLLIKIKECYDALSEKEKVFDKLESILKSLSTYDLSKIYISNDSQLSYISQKMFGRWDIISKAIREDCAKRNPQKSRESLEKFAERIDKKLKTIDSISIGDVDECLAQLGETYVKRVEDYFVAMGESEIDDEQTDTTSFKKNIEGAYESVKELLNNADNITDNNLMQDKGNVEKIKTLLDAIKDLQRFIKPLLGKGDEADKDGVFYGEFTSLWTKLDQVTPLYNMVRNYLTSKPYSTKKIKLNFENSTLMDGWDLNKEPDNTTVIFCKDGLYYLGIMGKKYNRVFVDREDLPHDGECYDKMEYKLLPGANKMLPKVFFSETGIQRFLPSEELLGKYERGTHKKGAGFDLGDCRALIDFFKKSIERHDDWKKFDFKFSDTSTYQDISEFYREVEQQGYKMSFRKVSVDYIKSLVEEGKLYLFQIYNKDFSAHSKGTPNMHTLYWKMLFDEENLKDVVYKLNGEAEVFFRKSSITVQSPTHPANSPIKNKNKDNQKKESKFEYDLIKDRRYTVDKFLFHVPITMNFKSVGGSNINQLVKRHIRSATDLHIIGIDRGERHLLYLTVIDSRGNIKEQFSLNEIVNEYNGNTYRTDYHELLDTREGERTEARRNWQTIQNIRELKEGYLSQVIHKISELAIKYNAVIVLEDLNFGFMRSRQKVEKQVYQKFEKMLIDKLNYLVDKKKPVAETGGLLRAYQLTGEFESFKTLGKQSGILFYVPAWNTSKIDPVTGFVNLFDTHYENIEKAKVFFDKFKSIRYNSDKDWFEFVVDDYTRFSPKAEGTRRDWTICTQGKRIQICRNHQRNNEWEGQEIDLTKAFKEHFEAYGVDISKDLREQINTQNKKEFFEELLRLLRLTLQMRNSMPSSDIDYLISPVANDTGCFFDSRKQAELKENAVLPMNADANGAYNIARKGLLAIRKMKQEENDSAKISLAISNKEWLKFAQTKPYLED

SEQ ID NO: 140 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 141 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKVLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRHYTMDKFQFHVPITMNFKCSAGSKVNDMVNAHIREAKDMHVIGIDRGERNLLYICVIDSRGTILDQISLNTINDIDYHDLLESRDKDRQQERRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHAQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 142 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKVLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQERRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHAQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 143 - protein, 1259 aa, Synergistes jonesii
MANSLKDFTNIYQLSKTLRFELKPIGKTEEHINRKLIIMHDEKRGEDYKSVTKLIDDYHRKFIHETLDPAHFDWNPLAEALIQSGSKNNKALPAEQKEMREKIISMFTSQAVYKKLFKKELFSELLPEMIKSELVSDLEKQAQLDAVKSFDKFSTYFTGFHENRKNIYSKKDTSTSIAFRIVHQNFPKFLANVRAYTLIKERAPEVIDKAQKELSGILGGKTLDDIFSIESFNNVLTQDKIDYYNQIIGGVSGKAGDKKLRGVNEFSNLYRQQHPEVASLRIKMVPLYKQILSDRTTLSFVPEALKDDEQAINAVDGLRSELERNDIFNRIKRLFGKNNLYSLDKIWIKNSSISAFSNELFKNWSFIEDALKEFKENEFNGARSAGKKAEKWLKSKYFSFADIDAAVKSYSEQVSADISSAPSASYFAKFTNLIETAAENGRKFSYFAAESKAFRGDDGKTEIIKAYLDSLNDILHCLKPFETEDISDIDTEFYSAFAEIYDSVKDVIPVYNAVRNYTTQKPFSTEKFKLNFENPALAKGWDKNKEQNNTAIILMKDGKYYLGVIDKNNKLRADDLADDGSAYGYMKMNYKFIPTPHMELPKVFLPKRAPKRYNPSREILLIKENKTFIKDKNFNRTDCHKLIDFFKDSINKHKDWRTFGFDFSDTDSYEDISDFYMEVQDQGYKLTFTRLSAEKIDKWVEEGRLFLFQIYNKDFADGAQGSPNLHTLYWKAIFSEENLKDVVLKLNGEAELFFRRKSIDKPAVHAKGSMKVNRRDIDGNPIDEGTYVEICGYANGKRDMASLNAGARGLIESGLVRITEVKHELVKDKRYTIDKYFFHVPFTINFKAQGQGNINSDVNLFLRNNKDVNIIGIDRGERNLVYVSLIDRDGHIKLQKDFNIIGGMDYHAKLNQKEKERDTARKSWKTIGTIKELKEGYLSQVVHEIVRLAVDNNAVIVMEDLNIGFKRGRFKVEKQVYQKFEKMLIDKLNYLVFKDAGYDAPCGILKGLQLTEKFESFTKLGKQCGIIFYIPAGYTSKIDPTTGFVNLFNINDVSSKEKQKDFIGKLDSIRFDAKRDMFTFEFDYDKFRTYQTSYRKKWAVWTNGKRIVREKDKDGKFRMNDRLLTEDMKNILNKYALAYKAGEDILPDVISRDKSLASEIFYVFKNTLQMRNSKRDTGEDFIISPVLNAKGRFFDSRKTDAALPIDADANGAYHIALKGSLVLDAIDEKLKEDGRIDYKDMAVSNPKWFEFMQTRKFDF

SEQ ID NO: 144 - protein, 1262 aa, Synergistes jonesii
MENMANSLKDFTNIYQLSKTLRFELKPIGKTEEHINRKLIIMHDEKRGEDYKSVTKLIDDYHRKFIHETLDPAHFDWNPLAEALIQSGSKNNKALPAEQKEMREKIISMFTSQAVYKKLFKKELFSELLPEMIKSELVSDLEKQAQLDAVKSFDKFSTYFTGFHENRKNIYSKKDTSTSIAFRIVHQNFPKFLANVRAYTLIKERAPEVIDKAQKELSGILGGKTLDDIFSIESFNNVLTQDKIDYYNQIIGGVSGKAGDKKLRGVNEFSNLYRQQHPEVASLRIKMVPLYKQILSDRTTLSFVPEALKDDEQAINAVDGLRSELERNDIFNRIKRLFGKNNLYSLDKIWIKNSSISAFSNELFKNWSFIEDALKEFKENEFNGARSAGKKAEKWLKSKYFSFADIDAAVKSYSEQVSADISSAPSASYFAKFTNLIETAAENGRKFSYFAAESKAFRGDDGKTEIIKAYLDSLNDILHCLKPFETEDISDIDTEFYSAFAEIYDSVKDVIPVYNAVRNYTTQKPFSTEKFKLNFENPALAKGWDKNKEQNNTAIILMKDGKYYLGVIDKNNKLRADDLADDGSAYGYMKMNYKFIPTPHMELPKVFLPKRAPKRYNPSREILLIKENKTFIKDKNFNRTDCHKLIDFFKDSINKHKDWRTFGFDFSDTDSYEDISDFYMEVQDQGYKLTFTRLSAEKIDKWVEEGRLFLFQIYNKDFADGAQGSPNLHTLYWKAIFSEENLKDVVLKLNGEAELFFRRKSIDKPAVHAKGSMKVNRRDIDGNPIDEGTYVEICGYANGKRDMASLNAGARGLIESGLVRITEVKHELVKDKRYTIDKYFFHVPFTINFKAQGQGNINSDVNLFLRNNKDVNIIGIDRGERNLVYVSLIDRDGHIKLQKDFNIIGGMDYHAKLNQKEKERDTARKSWKTIGTIKELKEGYLSQVVHEIVRLAVDNNAVIVMEDLNIGFKRGRFKVEKQVYQKFEKMLIDKLNYLVFKDAGYDAPCGILKGLQLTEKFESFTKLGKQCGIIFYIPAGYTSKIDPTTGFVNLFNINDVSSKEKQKDFIGKLDSIRFDAKRDMFTFEFDYDKFRTYQTSYRKKWAVWTNGKRIVREKDKDGKFRMNDRLLTEDMKNILNKYALAYKAGEDILPDVISRDKSLASEIFYVFKNTLQMRNSKRDTGEDFIISPVLNAKGRFFDSRKTDAALPIDADANGAYHIALKGSLVLDAIDEKLKEDGRIDYKDMAVSNPKWFEFMQTRKFDF

SEQ ID NO: 145 - protein, 1257 aa, Prevotella bryantii
MQINNLKIIYMKFTDFTGLYSLSKTLRFELKPIGKTLENIKKAGLLEQDQHRADSYKKVKKIIDEYHKAFIEKSLSNFELKYQSEDKLDSLEEYLMYYSMKRIEKTEKDKFAKIQDNLRKQIADHLKGDESYKTIFSKDLIRKNLPDFVKSDEERTLIKEFKDFTTYFKGFYENRENMYSAEDKSTAISHRIIHENLPKFVDNINAFSKIILIPELREKLNQIYQDFEEYLNVESIDEIFHLDYFSMVMTQKQIEVYNAIIGGKSTNDKKIQGLNEYINLYNQKHKDCKLPKLKLLFKQILSDRIAISWLPDNFKDDQEALDSIDTCYKNLLNDGNVLGEGNLKLLLENIDTYNLKGIFIRNDLQLTDISQKMYASWNVIQDAVILDLKKQVSRKKKESAEDYNDRLKKLYTSQESFSIQYLNDCLRAYGKTENIQDYFAKLGAVNNEHEQTINLFAQVRNAYTSVQAILTTPYPENANLAQDKETVALIKNLLDSLKRLQRFIKPLLGKGDESDKDERFYGDFTPLWETLNQITPLYNMVRNYMTRKPYSQEKIKLNFENSTLLGGWDLNKEHDNTAIILRKNGLYYLAIMKKSANKIFDKDKLDNSGDCYEKMVYKLLPGANKMLPKVFFSKSRIDEFKPSENIIENYKKGTHKKGANFNLADCHNLIDFFKSSISKHEDWSKFNFHFSDTSSYEDLSDFYREVEQQGYSISFCDVSVEYINKMVEKGDLYLFQIYNKDFSEFSKGTPNMHTLYWNSLFSKENLNNIIYKLNGQAEIFFRKKSLNYKRPTHPAHQAIKNKNKCNEKKESIFDYDLVKDKRYTVDKFQFHVPITMNFKSTGNTNINQQVIDYLRTEDDTHIIGIDRGERHLLYLVVIDSHGKIVEQFTLNEIVNEYGGNIYRTNYHDLLDTREQNREKARESWQTIENIKELKEGYISQVIHKITDLMQKYHAVVVLEDLNMGFMRGRQKVEKQVYQKFEEMLINKLNYLVNKKADQNSAGGLLHAYQLTSKFESFQKLGKQSGFLFYIPAWNTSKIDPVTGFVNLFDTRYESIDKAKAFFGKFDSIRYNADKDWFEFAFDYNNFTTKAEGTRTNWTICTYGSRIRTFRNQAKNSQWDNEEIDLTKAYKAFFAKHGINIYDNIKEAIAMETEKSFFEDLLHLLKLTLQMRNSITGTTTDYLISPVHDSKGNFYDSRICDNSLPANADANGAYNIARKGLMLIQQIKDSTSSNRFKFSPITNKDWLIFAQEKPYLND

SEQ ID NO: 146 - protein, 1253 aa, Prevotella albensis
MNIKNFTGLYPLSKTLRFELKPIGKTKENIEKNGILTKDEQRAKDYLIVKGFIDEYHKQFIKDRLWDFKLPLESEGEKNSLEEYQELYELTKRNDAQEADFTEIKDNLRSSITEQLTKSGSAYDRIFKKEFIREDLVNFLEDEKDKNIVKQFEDFTTYFTGFYENRKNMYSSEEKSTAIAYRLIHQNLPKFMDNMRSFAKIANSSVSEHFSDIYESWKEYLNVNSIEEIFQLDYFSETLTQPHIEVYNYIIGKKVLEDGTEIKGINEYVNLYNQQQKDKSKRLPFLVPLYKQILSDREKLSWIAEEFDSDKKMLSAITESYNHLHNVLMGNENESLRNLLLNIKDYNLEKINITNDLSLTEISQNLFGRYDVFTNGIKNKLRVLTPRKKKETDENFEDRINKIFKTQKSFSIAFLNKLPQPEMEDGKPRNIEDYFITQGAINTKSIQKEDIFAQIENAYEDAQVFLQIKDTDNKLSQNKTAVEKIKTLLDALKELQHFIKPLLGSGEENEKDELFYGSFLAIWDELDTITPLYNKVRNWLTRKPYSTEKIKLNFDNAQLLGGWDVNKEHDCAGILLRKNDSYYLGIINKKTNHIFDTDITPSDGECYDKIDYKLLPGANKMLPKVFFSKSRIKEFEPSEAIINCYKKGTHKKGKNFNLTDCHRLINFFKTSIEKHEDWSKFGFKFSDTETYEDISGFYREVEQQGYRLTSHPVSASYIHSLVKEGKLYLFQIWNKDFSQFSKGTPNLHTLYWKMLFDKRNLSDVVYKLNGQAEVFYRKSSIEHQNRIIHPAQHPITNKNELNKKHTSTFKYDIIKDRRYTVDKFQFHVPITINFKATGQNNINPIVQEVIRQNGITHIIGIDRGERHLLYLSLIDLKGNIIKQMTLNEIINEYKGVTYKTNYHNLLEKREKERTEARHSWSSIESIKELKDGYMSQVIHKITDMMVKYNAIVVLEDLNGGFMRGRQKVEKQVYQKFEKKLIDKLNYLVDKKLDANEVGGVLNAYQLTNKFESFKKIGKQSGFLFYIPAWNTSKIDPITGFVNLFNTRYESIKETKVFWSKFDIIRYNKEKNWFEFVFDYNTFTTKAEGTRTKWTLCTHGTRIQTFRNPEKNAQWDNKEINLTESFKALFEKYKIDITSNLKESIMQETEKKFFQELHNLLHLTLQMRNSVTGTDIDYLISPVADEDGNFYDSRINGKNFPENADANGAYNIARKGLMLIRQIKQADPQKKFKFETITNKDWLKFAQDKPYLKD

SEQ ID NO: 147 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYAWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 148 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEEYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKIYFRSGTSLTDVSRKVFGEWSIINRALDNYYATTYPIKPREKSEKYEERKEKWLKQDFNVSLIQTAIDEYDNETVKGKNSGKVIVDYFAKFCDDKETDLIQKVNEGYIAVKDLLNTPYPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKENLYYLGIMDKRHNRIFRNVPKADKKDSCYEKMVYKLLPGANKMLPKVFFSQSRIQEFTPSAKLLENYENETHKKGDNFNLNHCHQLIDFFKDSINKHEDWKNFDFRFSATSTYADLSGFYHEVEHQGYKISFQSIADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHVPITMNFKAEGIFNMNQRVNQFLKANPDINIIGIDRGERHLLYYTLINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLKQAKDFFEKFDSIRLNSKADYFEFAFDFKNFTGKADGGRTKWTVCTTNEDRYAWNRALNNNRGSQEKYDITAELKSLFDGKVDYKSGKDLKQQIASQELADFFRTLMKYLSVTLSLRHNNGEKGETEQDYILSPVADSMGKFFDSRKAGDDMPKNADANGAYHIALKGLWCLEQISKTDDLKKVKLAISNKEWLEFMQTLKG

SEQ ID NO: 149 - protein, 1247 aa, Prevotella bryantii
MKFTDFTGLYSLSKTLRFELKPIGKTLENIKKAGLLEQDQHRADSYKKVKKIIDEYHKAFIEKSLSNFELKYQSEDKLDSLEEYLMYYSMKRIEKTEKDKFAKIQDNLRKQIADHLKGDESYKTIFSKDLIRKNLPDFVKSDEERTLIKEFKDFTTYFKGFYENRENMYSAEDKSTAISHRIIHENLPKFVDNINAFSKIILIPELREKLNQIYQDFEEYLNVESIDEIFHLDYFSMVMTQKQIEVYNAIIGGKSTNDKKIQGLNEYINLYNQKHKDCKLPKLKLLFKQILSDRIAISWLPDNFKDDQEALDSIDTCYKNLLNDGNVLGEGNLKLLLENIDTYNLKGIFIRNDLQLTDISQKMYASWNVIQDAVILDLKKQVSRKKKESAEDYNDRLKKLYTSQESFSIQYLNDCLRAYGKTENIQDYFAKLGAVNNEHEQTINLFAQVRNAYTSVQAILTTPYPENANLAQDKETVALIKNLLDSLKRLQRFIKPLLGKGDESDKDERFYGDFTPLWETLNQITPLYNMVRNYMTRKPYSQEKIKLNFENSTLLGGWDLNKEHDNTAIILRKNGLYYLAIMKKSANKIFDKDKLDNSGDCYEKMVYKLLPGANKMLPKVFFSKSRIDEFKPSENIIENYKKGTHKKGANFNLADCHNLIDFFKSSISKHEDWSKFNFHFSDTSSYEDLSDFYREVEQQGYSISFCDVSVEYINKMVEKGDLYLFQIYNKDFSEFSKGTPNMHTLYWNSLFSKENLNNIIYKLNGQAEIFFRKKSLNYKRPTHPAHQAIKNKNKCNEKKESIFDYDLVKDKRYTVDKFQFHVPITMNFKSTGNTNINQQVIDYLRTEDDTHIIGIDRGERHLLYLVVIDSHGKIVEQFTLNEIVNEYGGNIYRTNYHDLLDTREQNREKARESWQTIENIKELKEGYISQVIHKITDLMQKYHAVVVLEDLNMGFMRGRQKVEKQVYQKFEEMLINKLNYLVNKKADQNSAGGLLHAYQLTSKFESFQKLGKQSGFLFYIPAWNTSKIDPVTGFVNLFDTRYESIDKAKAFFGKFDSIRYNADKDWFEFAFDYNNFTTKAEGTRTNWTICTYGSRIRTFRNQAKNSQWDNEEIDLTKAYKAFFAKHGINIYDNIKEAIAMETEKSFFEDLLHLLKLTLQMRNSITGTTTDYLISPVHDSKGNFYDSRICDNSLPANADANGAYNIARKGLMLIQQIKDSTSSNRFKFSPITNKDWLIFAQEKPYLND

SEQ ID NO: 150 - protein, 1247 aa, unidentified
MFKGDAFTGLYEVQKTLRFELVPIGLTQSYLENDWVIQKDKEVEENYGKIKAYFDLIHKEFVRQSLENAWLCQLDDFYEKYIELHNSLETRKDKNLAKQFEKVMKSLKKEFVSFFDAKWNEWKQKFSFLKKWWIDVLNEKEVLDLMAEFYPDEKELFDKFDKFFTYFSNFKESRKNFYADDGRAWAIATRAIDENLITFIKNIEDFKKLNSSFREFVNDNFSEEDKQIFEIDFYNNCLLQPWIDKYNKIVWWYSLENWEKVQWLNEKINNFKQNQNKSNSKDLKFPRMKLLYKQILGDKEKKVYIDEIRDDKNLIDLIDNSKRRNQIKIDNANDIINDFINNNAKFELDKIYLTRQSINTISSKYFSSWDYIRWYFWTGELQEFVSFYDLKETFWKIEYETLENIFKDCYVKGINTESQNNIVFETQGIYENFLNIFKFEFNQNISQISLLEWELDKIQNEDIKKNEKQVEVIKNYFDSVMSVYKMTKYFSLEKWKKRVELDTDNNFYNDFNEYLEGFEIWKDYNLVRNYITKKQVNTDKIKLNFDNSQFLTWWDKDKENERLGIILRREWKYYLWILKKWNTLNFGDYLQKEWEIFYEKMNYKQLNNVYRQLPRLLFPLTKKLNELKWDELKKYLSKYIQNFWYNEEIAQIKIEFDIFQESKEKWEKFDIDKLRKLIEYYKKWVLALYSDLYDLEFIKYKNYDDLSIFYSDVEKKMYNLNFTKIDKSLIDGKVKSWELYLFQIYNKDFSESKKEWSTENIHTKYFKLLFNEKNLQNLVVKLSWWADIFFRDKTENLKFKKDKNGQEILDHRRFSQDKIMFHISITLNANCWDKYWFNQYVNEYMNKERDIKIIWIDRWEKHLAYYCVIDKSWKIFNNEIWTLNELNWVNYLEKLEKIESSRKDSRISWWEIENIKELKNGYISQVINKLTELIVKYNAIIVFEDLNIWFKRWRQKIEKQIYQKLELALAKKLNYLTQKDKKDDEILWNLKALQLVPKVNDYQDIWNYKQSWIMFYVRANYTSVTCPNCWLRKNLYISNSATKENQKKSLNSIAIKYNDWKFSFSYEIDDKSWKQKQSLNKKKFIVYSDIERFVYSPLEKLTKVIDVNKKLLELFRDFNLSLDINKQIQEKDLDSVFFKSLTHLFNLILQLRNSDSKDNKDYISCPSCYYHSNNWLQWFEFNWDANWAYNIARKGIILLDRIRKNQEKPDLYVSDIDWDNFVQSNQFPNTIIPIQNIEKQVPLNIKI

SEQ ID NO: 151 - protein, 1246 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 152 - protein, 1241 aa, Butyrivibrio proteoclasticus
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEKARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 153 - protein, 1238 aa, unidentified
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDEVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIKRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLPRVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 154 - protein, 1235 aa, Anaerovibrio sp.
MVAFIDEFVGQYPVSKTLRFEARPVPETKKWLESDQCSVLFNDQKRNEYYGVLKELLDDYYRAYIEDALTSFTLDKALLENAYDLYCNRDTNAFSSCCEKLRKDLVKAFGNLKDYLLGSDQLKDLVKLKAKVDAPAGKGKKKIEVDSRLINWLNNNAKYSAEDREKYIKAIESFEGFVTYLTNYKQARENMFSSEDKSTAIAFRVIDQNMVTYFGNIRIYEKIKAKYPELYSALKGFEKFFSPTAYSEILSQSKIDEYNYQCIGRPIDDADFKGVNSLINEYRQKNGIKARELPVMSMLYKQILSDRDNSFMSEVINRNEEAIECAKNGYKVSYALFNELLQLYKKIFTEDNYGNIYVKTQPLTELSQALFGDWSILRNALDNGKYDKDIINLAELEKYFSEYCKVLDADDAAKIQDKFNLKDYFIQKNALDATLPDLDKITQYKPHLDAMLQAIRKYKLFSMYNGRKKMDVPENGIDFSNEFNAIYDKLSEFSILYDRIRNFATKKPYSDEKMKLSFNMPTMLAGWDYNNETANGCFLFIKDGKYFLGVADSKSKNIFDFKKNPHLLDKYSSKDIYYKVKYKQVSGSAKMLPKVVFAGSNEKIFGHLISKRILEIREKKLYTAAAGDRKAVAEWIDFMKSAIAIHPEWNEYFKFKFKNTAEYDNANKFYEDIDKQTYSLEKVEIPTEYIDEMVSQHKLYLFQLYTKDFSDKKKKKGTDNLHTMYWHGVFSDENLKAVTEGTQPIIKLNGEAEMFMRNPSIEFQVTHEHNKPIANKNPLNTKKESVFNYDLIKDKRYTERKFYFHCPITLNFRADKPIKYNEKINRFVENNPDVCIIGIDRGERHLLYYTVINQTGDILEQGSLNKISGSYTNDKGEKVNKETDYHDLLDRKEKGKHVAQQAWETIENIKELKAGYLSQVVYKLTQLMLQYNAVIVLENLNVGFKRGRTKVEKQVYQKFEKAMIDKLNYLVFKDRGYEMNGSYAKGLQLTDKFESFDKIGKQTGCIYYVIPSYTSHIDPKTGFVNLLNAKLRYENITKAQDTIRKFDSISYNAKADYFEFAFDYRSFGVDMARNEWVVCTCGDLRWEYSAKTRETKAYSVTDRLKELFKAHGIDYVGGENLVSHITEVADKHFLSTLLFYLRLVLKMRYTVSGTENENDFILSPVEYAPGKFFDSREATSTEPMNADANGAYHIALKGLMTIRGIEDGKLHNYGKGGENAAWFKFMQNQEYKNNG

SEQ ID NO: 155 - protein, 1233 aa, unidentified
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKLLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 156 - protein, 1235 aa, unidentified
MTMDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKLLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 157 - protein, 1231 aa, Butyrivibrio fibrisolvens
MYYESLTKLYPIKKTIRNELVPIGKTLENIKKNNILEADEDRKIAYIRVKAIMDDYHKRLINEALSGFALIDLDKAANLYLSRSKSADDIESFSRFQDKLRKAIAKRLREHENFGKIGNKDIIPLLQKLSENEDDYNALESFKNFYTYFESYNDVRLNLYSDKEKSSTVAYRLINENLPRFLDNIRAYDAVQKAGITSEELSSEAQDGLFLVNTFNNVLIQDGINTYNEDIGKLNVAINLYNQKNASVQGFRKVPKMKVLYKQILSDREESFIDEFESDTELLDSLESHYANLAKYFGSNKVQLLFTALRESKGVNVYVKNDIAKTSFSNVVFGSWSRIDELINGEYDDNNNRKKDEKYYDKRQKELKKNKSYTIEKIITLSTEDVDVIGKYIEKLESDIDDIRFKGKNFYEAVLCGHDRSKKLSKNKGAVEAIKGYLDSVKDFERDLKLINGSGQELEKNLVVYGEQEAVLSELSGIDSLYNMTRNYLTKKPFSTEKIKLNFNKPTFLDGWDYGNEEAYLGFFMIKEGNYFLAVMDANWNKEFRNIPSVDKSDCYKKVIYKQISSPEKSIQNLMVIDGKTVKKNGRKEKEGIHSGENLILEELKNTYLPKKINDIRKRRSYLNGDTFSKKDLTEFIGYYKQRVIEYYNGYSFYFKSDDDYASFKEFQEDVGRQAYQISYVDVPVSFVDDLINSGKLYLFRVYNKDFSEYSKGRLNLHTLYFKMLFDERNLKNVVYKLNGQAEVFYRPSSIKKEELIVHRAGEEIKNKNPKRAAQKPTRRLDYDIVKDRRYSQDKFMLHTSIIMNFGAEENVSFNDIVNGVLRNEDKVNVIGIDRGERNLLYVVVIDPEGKILEQRSLNCITDSNLDIETDYHRLLDEKESDRKIARRDWTTIENIKELKAGYLSQVVHIVAELVLKYNAIICLEDLNFGFKRGRQKVEKQVYQKFEKMLIDKLNYLVMDKSREQLSPEKISGALNALQLTPDFKSFKVLGKQTGIIYYVPAYLTSKIDPMTGFANLFYVKYENVDKAKEFFSKFDSIKYNKDGKNWNTKGYFEFAFDYKKFTDRAYGRVSEWTVCTVGERIIKFKNKEKNNSYDDKVIDLTNSLKELFDSYKVTYESEVDLKDAILAIDDPAFYRDLTRRLQQTLQMRNSSCDGSRDYIISPVKNSKGEFFCSDNNDDTTPNDADANGAFNIARKGLWVLNEIRNSEEGSKINLAMSNAQWLEYAQDNTI

SEQ ID NO: 158 - protein, 1230 aa, unidentified
MHENNGKIADNFIGIYPVSKTLRFELKPVGKTQEYIEKHGILDEDLKRAGDYKSVKKIIDAYHKYFIDEALNGIQLDGLKNYYELYEKKRDNNEEKEFQKIQMSLRKQIVKRFSEHPQYKYLFKKELIKNVLPEFTKDNAEEQTLVKSFQEFTTYFEGFHQNRKNMYSDEEKSTAIAYRVVHQNLPKYIDNMRIFSMILNTDIRSDLTELFNNLKTKMDITIVEEYFAIDGFNKVVNQKGIDVYNTILGAFSTDDNTKIKGLNEYINLYNQKNKAKLPKLKPLFKQILSDRDKISFIPEQFDSDTEVLEAVDMFYNRLLQFVIENEGQITISKLLTNFSAYDLNKIYVKNDTTISAISNDLFDDWSYISKAVRENYDSENVDKNKRAAAYEEKKEKALSKIKMYSIEELNFFVKKYSCNECHIEGYFERRILEILDKMRYAYESCKILHDKGLINNISLCQDRQAISELKDFLDSIKEVQWLLKPLMIGQEQADKEEAFYTELLRIWEELEPITLLYNKVRNYVTKKPYTLEKVKLNFYKSTLLDGWDKNKEKDNLGIILLKDGQYYLGIMNRRNNKIADDAPLAKTDNVYRKMEYKLLTKVSANLPRIFLKDKYNPSEEMLEKYEKGTHLKGENFCIDDCRELIDFFKKGIKQYEDWGQFDFKFSDTESYDDISAFYKEVEHQGYKITFRDIDETYIDSLVNEGKLYLFQIYNKDFSPYSKGTKNLHTLYWEMLFSQQNLQNIVYKLNGNAEIFYRKASINQKDVVVHKADLPIKNKDPQNSKKESMFDYDIIKDKRFTCDKYQFHVPITMNFKALGENHFNRKVNRLIHDAENMHIIGIDRGERNLIYLCMIDMKGNIVKQISLNEIISYDKNKLEHKRNYHQLLKTREDENKSARQSWQTIHTIKELKEGYLSQVIHVITDLMVEYNAIVVLEDLNFGFKQGRQKFERQVYQKFEKMLIDKLNYLVDKSKGMDEDGGLLHAYQLTDEFKSFKQLGKQSGFLYYIPAWNTSKLDPTTGFVNLFYTKYESVEKSKEFINNFTSILYNQEREYFEFLFDYSAFTSKAEGSRLKWTVCSKGERVETYRNPKKNNEWDTQKIDLTFELKKLFNDYSISLLDGDLREQMGKIDKADFYKKFMKLFALIVQMRNSDEREDKLISPVLNKYGAFFETGKNERMPLDADANGAYNIARKGLWIIEKIKNTDVEQLDKVKLTISNKEWLQYAQEHIL

SEQ ID NO: 159 - protein, 1228 aa, unidentified
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 160 - protein, 1246 aa, unidentified
MLKNVGIDRLDVEKGRKNMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 161 - protein, 1227 aa, unidentified
MDAKEFTGQYPLSKTLRFELRPIGRTWDNLEASGYLAEDRHRAECYPRAKELLDDNHRAFLNRVLPQIDMDWHPIAEAFCKVHKNPGNKELAQDYNLQLSKRRKEISAYLQDADGYKGLFAKPALDEAMKIAKENGNESDIEVLEAFNGFSVYFTGYHESRENIYSDEDMVSVAYRITEDNFPRFVSNALIFDKLNESHPDIISEVSGNLGVDDIGKYFDVSNYNNFLSQAGIDDYNHIIGGHTTEDGLIQAFNVVLNLRHQKDPGFEKIQFKQLYKQILSVRTSKSYIPKQFDNSKEMVDCICDYVSKIEKSETVERALKLVRNISSFDLRGIFVNKKNLRILSNKLIGDWDAIETALMHSSSSENDKKSVYDSAEAFTLDDIFSSVKKFSDASAEDIGNRAEDICRVISETAPFINDLRAVDLDSLNDDGYEAAVSKIRESLEPYMDLFHELEIFSVGDEFPKCAAFYSELEEVSEQLIEIIPLFNKARSFCTRKRYSTDKIKVNLKFPTLADGWDLNKERDNKAAILRKDGKYYLAILDMKKDLSSIRTSDEDESSFEKMEYKLLPSPVKMLPKIFVKSKAAKEKYGLTDRMLECYDKGMHKSGSAFDLGFCHELIDYYKRCIAEYPGWDVFDFKFRETSDYGSMKEFNEDVAGAGYYMSLRKIPCSEVYRLLDEKSIYLFQIYNKDYSENAHGNKNMHTMYWEGLFSPQNLESPVFKLSGGAELFFRKSSIPNDAKTVHPKGSVLVPRNDVNGRRIPDSIYRELTRYFNRGDCRISDEAKSYLDKVKTKKADHDIVKDRRFTVDKMMFHVPIAMNFKAISKPNLNKKVIDGIIDDQDLKIIGIDRGERNLIYVTMVDRKGNILYQDSLNILNGYDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTTLASVGKQCGVIFYIPAAFTSKIDPTTGFADLFALSNVKNVASMREFFSKMKSVIYDKAEGKFAFTFDYLDYNVKSECGRTLWTVYTVGERFTYSRVNREYVRKVPTDIIYDALQKAGISVEGDLRDRIAESDGDTLKSIFYAFKYALDMRVENREEDYIQSPVKNASGEFFCSKNAGKSLPQDSDANGAYNIALKGILQLRMLSEQYDPNAESIRLPLITNKAWLTFMQSGMKTWKN

SEQ ID NO: 162 - protein, 1224 aa, unidentified
MGLYDGFVNRYSVSKTLRFELIPQGRTREYIETNGILSDDEERAKDYKTIKRLIDEYHKDYISRCLKNVNISCLEEYYHLYNSSNRDKRHEELDALSDQMRGEIASFLTGNDEYKEQKSRDIIINERIINFASTDEELAAVKRFRKFTSYFTGFFTNRENMYSAEKKSTAIAHRIIDVNLPKYVDNIKAFNTAIEAGVFDIAEFESNFKAITDEHEVSDLLDITKYSRFIRNEDIIIYNTLLGGISMKDEKIQGLNELINLHNQKHPGKKVPLLKVLYKQILGDSQTHSFVDDQFEDDQQVINAVKAVTDTFSETLLGSLKIIINNIGHYDLDRIYIKAGQDITTLSKRALNDWHIITECLESEYDDKFPKNKKSDTYEEMRNRYVKSFKSFSIGRLNSLVTTYTEQACFLENYLGSFGGDTDKNCLTDFTNSLMEVEHLLNSEYPVTNRLITDYESVRILKRLLDSEMEVIHFLKPLLGNGNESDKDLVFYGEFEAEYEKLLPVIKVYNRVRNYLTRKPFSTEKIKLNFNSPTLLCGWSQSKEKEYMGVILRKDGQYYLGIMTPSNKKIFSEAPKPDEDCYEKMVLRYIPHPYQMLPKVFFSKSNIAFFNPSDEILRIKKQESFKKGKSFNRDDCHKFIDFYKDSINRHEEWRKFNFKFSDTDSYEDISRFYKEVENQAFSMSFTKIPTVYIDSLVDEGKLYLFKLHNKDFSEHSKGKPNLHTVYWNALFSEYNLQNTVYQLNGSAEIFFRKASIPENERVIHKKNVPITRKVAELNGKKEVSVFPYDIIKNRRYTVDKFQFHVPLKMNFKADEKKRINDDVIEAIRSNKGIHVIGIDRGERNLLYLSLINEEGRIIEQRSLNIIDSGEGHTQNYRDLLDSREKDREKARENWQEIQEIKDLKTGYLSQAIHTITKWMKEYNAIIVLEDLNDRFTNGRKKVEKQVYQKFEKMLIDKLNYYVDKDEEFDRMGGTHRALQLTEKFESFQKLGRQTGFIFYVPAWNTSKLDPTTGFVDLLYPKYKSVDATKDFIKKFDFIRFNSEKNYFEFGLHYSNFTERAIGCRDEWILCSYGNRIVNFRNAAKNNSWDYKEIDITKQLLDLFEKNGIDVKQENLIDSICEMKDKPFFKSLIANIKLILQIRNSASGTDIDYMISPAMNDRGEFFDTRKGLQQLPLDADANGAYNIAKKGLWIVDQIRNTTGNNVKMAMSNREWMHFAQESRLA

SEQ ID NO: 163 - protein, 1214 aa, unidentified
MKNVFGGFTNLYSLTKTLRFELKPTSKTQKLMKRNNVIQTDEEIDKLYHDEMKPILDEIHRRFINDALAQKIFISASLDNFLKVVKNYKVESAKKNIKQNQVKLLQKEITIKTLGLRREVVSGFITVSKKWKDKYVGLGIKLKGDGYKVLTEQAVLDILKIEFPNKAKYIDKFRGFWTYFSGFNENRKNYYSEEDKATSIANRIVNENLSRYIDNIIAFEEILQKIPNLKKFKQDLDITSYNYYLNQAGIDKYNKIIGGYIVDKDKKIQGINEKVNLYTQQTKKKLPKLKFLFKQIGSERKGFGIFEIKEGKEWEQLGDLFKLQRTKINSNGREKGLFDSLRTMYREFFDEIKRDSNSQARYSLDKIYFNKASVNTISNSWFTNWNKFAELLNIKEDKKNGEKKIPEQISIEDIKDSLSIIPKENLEELFKLTNREKHDRTRFFGSNAWVTFLNIWQNEIEESFNKLEEKEKDFKKNAAIKFQKNNLVQKNYIKEVCDRMLAIERMAKYHLPKDSNLSREEDFYWIIDNLSEQREIYKYYNAFRNYISKKPYNKSKMKLNFENGNLLGGWSDGQERNKAGVILRNGNKYYLGVLINRGIFRTDKINNEIYRTGSSKWERLILSNLKFQTLAGKGFLGKHGVSYGNMNPEKSVPSLQKFIRENYLKKYPQLTEVSNTKFLSKKDFDAAIKEALKECFTMNFINIAENKLLEAEDKGDLYLFEITNKDFSGKKSGKDNIHTIYWKYLFSESNCKSPIIGLNGGAEIFFREGQKDKLHTKLDKKGKKVFDAKRYSEDKLFFHVSITINYGKPKNIKFRDIINQLITSMNVNIIGIDRGEKHLLYYSVIDSNGIILKQGSLNKIRVGDKEVDFNKKLTERANEMKKARQSWEQIGNIKNFKEGYLSQAIHEIYQLMIKYNAIIVLEDLNTEFKAKRLSKVEKSVYKKFELKLARKLNHLILKDRNTNEIGGVLKAYQLTPTIGGGDVSKFEKAKQWGMMFYVRANYTSTTDPVTGWRKHLYISNFSNNSVIKSFFDPTNRDTGIEIFYSGKYRSWGFRYVQKETGKKWELFATKELERFKYNQTTKLCEKINLYDKFEELFKGIDKSADIYSQLCNVLDFRWKSLVYLWNLLNQIRNVDKNAEGNKNDFIQSPVYPFFDSRKTDGKTEPINGDANGALNIARKGLMLVERIKNNPEKYEQLIRDTEWDAWIQNFNKVN

SEQ ID NO: 164 - protein, 1206 aa, unidentified
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 165 - protein, 1206 aa, Butyrivibrio sp.
MYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 166 - protein, 1206 aa, unidentified
MENYYDSLTRQYPVTKTIRQELKPVGKTLENIKNAEIIEADKQKKEAYVKVKELMDEFHKSIIEKSLVGIKLDGLSEFEKLYKIKTKTDEDKNRISELFYYMRKQIADALKNSRDYGYVDNKDLIEKILPERVKDENSLNALSCFKGFTTYFTDYYKNRKNIYSDEEKHSTVGYRCINENLLIFMSNIEVYQIYKKANIKNDNYDEETLDKTFMIESFNECLTQSGVEAYNSVVASIKTATNLYIQKNNKEENFVRVPKMKVLFKQILSDRTSLFDGLIIESDDELLDKLCSFSAEVDKFLPINIDRYIKTLMDSNNGTGIYVKNDSSLTTLSNYLTDSWSSIRNAFNENYDAKYTGKVNDKYEEKREKAYKSNDSFELNYIQNLLGINVIDKYIERINFDIKEICEAYKEMTKNCFEDHDKTKKLQKNIKAVASIKSYLDSLKNIERDIKLLNGTGLESRNEFFYGEQSTVLEEITKVDELYNITRNYLTKKPFSTEKMKLNFNNPQLLGGWDVNKERDCYGVILIKDNNYYLGIMDKSANKSFLNIKESKNENAYKKVNCKLLPGPNKMFPKVFFAKSNIDYYDPTHEIKKLYDKGTFKKGNSFNLEDCHKLIDFYKESIKKNDDWKNFNFNFSDTKDYEDISGFFREVEAQNYKITYTNVSCDFIESLVDEGKLYLFQIYNKDFSEYATGNLNLHTLYLKMLFDERNLKDLCIKMNGEAEVFYRPASILDEDKVVHKANQKITNKNTNSKKKESIFSYDIVKDKRYTVDKFFIHLPITLNYKEQNVSRFNDYIREILKKSKNIRVIGIDRGERNLLYVVVCDSDGSILYQRSINEIVSGSHKTDYHKLLDNKEKERLSSRRDWKTIENIKDLKAGYMSQVVNEIYNLILKYNAIVVLEDLNIGFKNGRKKVEKQVYQNFEKALIDKLNYLCIDKTREQLSPSSPGGVLNAYQLTAKFESFEKIGKQTGCIFYVPAYLTSQIDPTTGFVNLFYQKDTSKQGLQLFFRKFKKINFDKVASNFEFVFDYNDFTNKAEGTKTNWTISTQGTRIAKYRSDDANGKWISRTVHPTDIIKEALNREKINYNDGHDLIDEIVSIEKSAVLKEIYYGFKLTLQLRNSTLANEEEQEDYIISPVKNSSGNYFDSRITSKELPCDADANGAYNIARKGLWALEQIRNSENVSKVKLAISNKEWFEYTQNNIPSL

SEQ ID NO: 167 - protein, 1222 aa, Oribacterium sp.
METEILKYDFFEREGKYMYYDGLTKQYALSKTIRNELVPIGKTLDNIKKNRILEADIKRKSDYEHVKKLMDMYHKKIINEALDNFKLSVLEDAADIYFNKQNDERDIDAFLKIQDKLRKEIVEQLKGHTDYSKVGNKDFLGLLKAASTEEDRILIESFDNFYTYFTSYNKVRSNLYSAEDKSSTVAYRLINENLPKFFDNIKAYRTVRNAGVISGDMSIVEQDELFEVDTFNHTLTQYGIDTYNHMIGQLNSAINLYNQKMHGAGSFKKLPKMKELYKQLLTEREEEFIEEYTDDEVLITSVHNYVSYLIDYLNSDKVESFFDTLRKSDGKEVFIKNDVSKTTMSNILFDNWSTIDDLINHEYDSAPENVKKTKDDKYFEKRQKDLKKNKSYSLSKIAALCRDTTILEKYIRRLVDDIEKIYTSNNVFSDIVLSKHDRSKKLSKNTNAVQAIKNMLDSIKDFEHDVMLINGSGQEIKKNLNVYSEQEALAGILRQVDHIYNLTRNYLTKKPFSTEKIKLNFNRPTFLDGWDKNKEEANLGILLIKDNRYYLGIMNTSSNKAFVNPPKAISNDIYKKVDYKLLPGPNKMLPKVFFATKNIAYYAPSEELLSKYRKGTHKKGDSFSIDDCRNLIDFFKSSINKNTDWSTFGFNFSDTNSYNDISDFYREVEKQGYKLSFTDIDACYIKDLVDNNELYLFQIYNKDFSPYSKGKLNLHTLYFKMLFDQRNLDNVVYKLNGEAEVFYRPASIESDEQIIHKSGQNIKNKNQKRSNCKKTSTFDYDIVKDRRYCKDKFMLHLPITVNFGTNESGKFNELVNNAIRADKDVNVIGIDRGERNLLYVVVVDPCGKIIEQISLNTIVDKEYDIETDYHQLLDEKEGSRDKARKDWNTIENIKELKEGYLSQVVNIIAKLVLKYDAIICLEDLNFGFKRGRQKVEKQVYQKFEKMLIDKMNYLVLDKSRKQESPQKPGGALNALQLTSAFKSFKELGKQTGIIYYVPAYLTSKIDPTTGFANLFYIKYESVDKARDFFSKFDFIRYNQMDNYFEFGFDYKSFTERASGCKSKWIACTNGERIVKYRNSDKNNSFDDKTVILTDEYRSLFDKYLQNYIDEDDLKDQILQIDSADFYKNLIKLFQLTLQMRNSSSDGKRDYIISPVKNYREEFFCSEFSDDTFPRDADANGAYNIARKGLWVIKQIRETKSGTKINLAMSNSEWLEYAQCNLL

SEQ ID NO: 168 - protein, 1205 aa, Pseudobutyrivibrio ruminis
MYYQNLTKMYPISKTLRNELIPVGKTLENIRKNGILEADIQRKADYEHVKKLMDNYHKQLINEALQGVHLSDLSDAYDLYFNLSKEKNSVDAFSKCQDKLRKEIVSLLKNHENFPKIGNKEIIKLLQSLYDNDTDYKALDSFSNFYTYFSSYNEVRKNLYSDEEKSSTVAYRLINENLPKFLDNIKAYAIAKKAGVRAEGLSEEDQDCLFIIETFERTLTQDGIDNYNAAIGKLNTAINLFNQQNKKQEGFRKVPQMKCLYKQILSDREEAFIDEFSDDEDLITNIESFAENMNVFLNSEIITDFKIALVESDGSLVYIKNDVSKTSFSNIVFGSWNAIDEKLSDEYDLANSKKKKDEKYYEKRQKELKKNKSYDLETIIGLFDDNSDVIGKYIEKLESDITAIAEAKNDFDEIVLRKHDKNKSLRKNTNAVEAIKSYLDTVKDFERDIKLINGSGQEVEKNLVVYAEQENILAEIKNVDSLYNMSRNYLTQKPFSTEKFKLNFNRATLLNGWDKNKETDNLGILFEKDGMYYLGIMNTKANKIFVNIPKATSNDVYHKVNYKLLPGPNKMLPKVFFAQSNLDYYKPSEELLAKYKAGTHKKGDNFSLEDCHALIDFFKASIEKHPDWSSFGFEFSETCTYEDLSGFYREVEKQGYKITYTDVDADYITSLVERDELYLFQIYNKDFSPYSKGNLNLHTIYLQMLFDQRNLNNVVYKLNGEAEVFYRPASINDEEVIIHKAGEEIKNKNSKRAVDKPTSKFGYDIIKDRRYSKDKFMLHIPVTMNFGVDETRRFNDVVNDALRNDEKVRVIGIDRGERNLLYVVVVDTDGTILEQISLNSIINNEYSIETDYHKLLDEKEGDRDRARKNWTTIENIKELKEGYLSQVVNVIAKLVLKYNAIICLEDLNFGFKRGRQKVEKQVYQKFEKMLIDKLNYLVIDKSRKQDKPEEFGGALNALQLTSKFTSFKDMGKQTGIIYYVPAYLTSKIDPTTGFANLFYVKYENVEKAKEFFSRFDSISYNNESGYFEFAFDYKKFTDRACGARSQWTVCTYGERIIKFRNTEKNNSFDDKTIVLSEEFKELFSIYGISYEDGAELKNKIMSVDEADFFRSLTRLFQQTMQMRNSSNDVTRDYIISPIMNDRGEFFNSEACDASKPKDADANGAFNIARKGLWVLEQIRNTPSGDKLNLAMSNAEWLEYAQRNQI

SEQ ID NO: 169 - protein, 1154 aa, Proteocatella sphenisci
MENFKNLYPINKTLRFELRPYGKTLENFKKSGLLEKDAFKANSRRSMQAIIDEKFKETIEERLKYTEFSECDLGNMTSKDKKITDKAATNLKKQVILSFDDEIFNNYLKPDKNIDALFKNDPSNPVISTFKGFTTYFVNFFEIRKHIFKGESSGSMAYRIIDENLTTYLNNIEKIKKLPEELKSQLEGIDQIDKLNNYNEFITQSGITHYNEIIGGISKSENVKIQGINEGINLYCQKNKVKLPRLTPLYKMILSDRVSNSFVLDTIENDTELIEMISDLINKTEISQDVIMSDIQNIFIKYKQLGNLPGISYSSIVNAICSDYDNNFGDGKRKKSYENDRKKHLETNVYSINYISELLTDTDVSSNIKMRYKELEQNYQVCKENFNATNWMNIKNIKQSEKTNLIKDLLDILKSIQRFYDLFDIVDEDKNPSAEFYTWLSKNAEKLDFEFNSVYNKSRNYLTRKQYSDKKIKLNFDSPTLAKGWDANKEIDNSTIIMRKFNNDRGDYDYFLGIWNKSTPANEKIIPLEDNGLFEKMQYKLYPDPSKMLPKQFLSKIWKAKHPTTPEFDKKYKEGRHKKGPDFEKEFLHELIDCFKHGLVNHDEKYQDVFGFNLRNTEDYNSYTEFLEDVERCNYNLSFNKIADTSNLINDGKLYVFQIWSKDFSIDSKGTKNLNTIYFESLFSEENMIEKMFKLSGEAEIFYRPASLNYCEDIIKKGHHHAELKDKFDYPIIKDKRYSQDKFFFHVPMVINYKSEKLNSKSLNNRTNENLGQFTHIIGIDRGERHLIYLTVVDVSTGEIVEQKHLDEIINTDTKGVEHKTHYLNKLEEKSKTRDNERKSWEAIETIKELKEGYISHVINEIQKLQEKYNALIVMENLNYGFKNSRIKVEKQVYQKFETALIKKFNYIIDKKDPETYIHGYQLTNPITTLDKIGNQSGIVLYIPAWNTSKIDPVTGFVNLLYADDLKYKNQEQAKSFIQKIDNIYFENGEFKFDIDFSKWNNRYSISKTKWTLTSYGTRIQTFRNPQKNNKWDSAEYDLTEEFKLILNIDGTLKSQDVETYKKFMSLFKLMLQLRNSVTGTDIDYMISPVTDKTGTHFDSRENIKNLPADADANGAYNIARKGIMAIENIMNGISDPLKISNEDYLKYIQNQQE

SEQ ID NO: 170 - protein, 1171 aa, Proteocatella sphenisci
MIILYISTSNMNMEGVFMENFKNLYPINKTLRFELRPYGKTLENFKKSGLLEKDAFKANSRRSMQAIIDEKFKETIEERLKYTEFSECDLGNMTSKDKKITDKAATNLKKQVILSFDDEIFNNYLKPDKNIDALFKNDPSNPVISTFKGFTTYFVNFFEIRKHIFKGESSGSMAYRIIDENLTTYLNNIEKIKKLPEELKSQLEGIDQIDKLNNYNEFITQSGITHYNEIIGGISKSENVKIQGINEGINLYCQKNKVKLPRLTPLYKMILSDRVSNSFVLDTIENDTELIEMISDLINKTEISQDVIMSDIQNIFIKYKQLGNLPGISYSSIVNAICSDYDNNFGDGKRKKSYENDRKKHLETNVYSINYISELLTDTDVSSNIKMRYKELEQNYQVCKENFNATNWMNIKNIKQSEKTNLIKDLLDILKSIQRFYDLFDIVDEDKNPSAEFYTWLSKNAEKLDFEFNSVYNKSRNYLTRKQYSDKKIKLNFDSPTLAKGWDANKEIDNSTIIMRKFNNDRGDYDYFLGIWNKSTPANEKIIPLEDNGLFEKMQYKLYPDPSKMLPKQFLSKIWKAKHPTTPEFDKKYKEGRHKKGPDFEKEFLHELIDCFKHGLVNHDEKYQDVFGFNLRNTEDYNSYTEFLEDVERCNYNLSFNKIADTSNLINDGKLYVFQIWSKDFSIDSKGTKNLNTIYFESLFSEENMIEKMFKLSGEAEIFYRPASLNYCEDIIKKGHHHAELKDKFDYPIIKDKRYSQDKFFFHVPMVINYKSEKLNSKSLNNRTNENLGQFTHIIGIDRGERHLIYLTVVDVSTGEIVEQKHLDEIINTDTKGVEHKTHYLNKLEEKSKTRDNERKSWEAIETIKELKEGYISHVINEIQKLQEKYNALIVMENLNYGFKNSRIKVEKQVYQKFETALIKKFNYIIDKKDPETYIHGYQLTNPITTLDKIGNQSGIVLYIPAWNTSKIDPVTGFVNLLYADDLKYKNQEQAKSFIQKIDNIYFENGEFKFDIDFSKWNNRYSISKTKWTLTSYGTRIQTFRNPQKNNKWDSAEYDLTEEFKLILNIDGTLKSQDVETYKKFMSLFKLMLQLRNSVTGTDIDYMISPVTDKTGTHFDSRENIKNLPADADANGAYNIARKGIMAIENIMNGISDPLKISNEDYLKYIQNQQE

SEQ ID NO: 171 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 172 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKEEKTQVIKLFSRFATSFKDYFKNRANCFSADDISSSSCHRIVNDNAEIFFSNALVYRRIVKNLSNDDINKISGDMKDSLKEMSLDEIYSYEKYGEFITQEGISFYNDICGKVNSFMNLYCQKNKENKNLYKLRKLHKQILCIADTSYEVPYKFESDEEVYQSVNGFLDNISSKHIVERLRKIGDNYNGYNLDKIYIVSRFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHLKSSKDFDITFCRDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKTIPENIYQELYKYFNDKSDKELSDEAAKLKNVVGHHEAATNIVKDYRYTYDKYFLHMPITINFKANKTSFINDRILQYIAKENDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPEKLKNVGHQCGCIFYVPAAYTSKIDPTTGFANIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFKLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 173 - protein, 1263 aa, Eubacterium rectale
MNNGTNNFQNFIGISSLQKTLRNALTPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKEEKTQVIKLFSRFATSFKDYFKNRANCFSADDISSSSCHRIVNDNAEIFFSNALVYRRIVKNLSNDDINKISGDMKDSLKKMSLEKIYSYEKYGEFITQEGISFYNDICGKVNSFMNLYCQKNKENKNLYKLRKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPEKKIIEGNTSENKGDYKKMIYNLLPGPNKMIPKVFLSSKTGVETYKPSAYILEGYKQNKHLKSSKDFDITFCRDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDVVLKLNGEAEIFFRKSSIKNPIIHKKGSILVNRTYEAEEKDQFGNIQIVRKTIPENIYQELYKYFNDKSDKELSDEAAKLKNAVGHHEAATNIVKDYRYTYDKYFLHMPITINFKANKTSFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPEKLKNVGHQCGCIFYVPAAYTSKIDPTTGFVNIFKFKDLTVDAKREFIKKFDSIRYDSDKNLFCFTFDYNNFITQNTVMSKSSWSVYTYGVRIKRRFVNGRFSNESDTIDITKDMEKTLEMTDINWRDGHDLRQDIIDYEIVQHIFEIFKLTVQMRNSLSELEDRNYDRLISPVLNENNIFYDSAKAGDALPKDADANGAYCIALKGLYEIKQITENWKEDGKFSRDKLKISNKDWFDFIQNKRYL

SEQ ID NO: 174 - protein, 1263 aa, Eubacterium sp.
MNNGTNNFQNFIGISSLQKTLRNALIPTETTQQFIVKNGIIKEDELRGKNRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKKEKTQVIKLFSRFATSFKDYFKNRANCFSADDISSSSCHRIVNDNAEIFFSNALVYRRIVKNLSNDDINKISGDMKDSLKEMSLEEIYSYEKYGEFITQEGISFYNDICGKVNSFMNLYCQKNKENKNLYKLRKLHKQILCIADTSYEVPYKFESDEEVYQSVNGFLDNISSKHIVERLRKIGDNYNDYNLDKIYIVSKFYESVSQKTYRDWETINTALEIHYNNILPGNGKSKADKVKKAVKNDLQKSITEINELVSNYKLCSDDNIKAETYIHEISHILNNFEAHELKYNPEIHLVESELKASELKNVLDIIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHLKSSKDFDITFCHDLIDYFKNCIAIHPEWKNFGFDFSDTSAYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKTIPENIYQELYKYFNDKSDKELSDEAAKLKNVVGHHEAATNIVKDYRYTYDKYFLHMPITINFKANKTSFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFKLTVQMRNSLSELEDRDYDRLISPVLNENNIFYDSAKAGYALPKDADANGAYCIALKGLYEIKQITENWKEDGKFSRDKLKISNKDWFDFIQNKRYL

SEQ ID NO: 175 - protein, 1145 aa, Alicyclobacillus macrosporangiidus
MVAVKSIKVKLMLGHLPEIREGLWHLHEAVNLGVRYYTEWLALLRQGNLYRRGKDGAQECYMTAEQCRQELLVRLRDRQKRNGHTGDPGTDEELLGVARRLYELLVPQSVGKKGQAQMLASGFLSPLADPKSEGGKGTSKSGRKPAWMGMKEAGDSRWVEAKARYEANKAKDPTKQVIASLEMYGLRPLFDVFTETYKTIRWMPLGKHQGVRAWDRDMFQQSLERLMSWESWNERVGAEFARLVDRRDRFREKHFTGQEHLVALAQRLEQEMKEASPGFESKSSQAHRITKRALRGADGIIDDWLKLSEGEPVDRFDEILRKRQAQNPRRFGSHDLFLKLAEPVFQPLWREDPSFLSRWASYNEVLNKLEDAKQFATFTLPSPCSNPVWARFENAEGTNIFKYDFLFDHFGKGRHGVRFQRMIVMRDGVPTEVEGIVVPIAPSRQLDALAPNDAASPIDVFVGDPAAPGAFRGQFGGAKIQYRRSALVRKGRREEKAYLCGFRLPSQRRTGTPADDAGEVFLNLSLRVESQSEQAGRRNPPYAAVFHISDQTRRVIVRYGEIERYLAEHPDTGIPGSRGLTSGLRVMSVDLGLRTSAAISVFRVAHRDELTPDAHGRQPFFFPIHGMDHLVALHERSHLIRLPGETESKKVRSIREQRLDRLNRLRSQMASLRLLVRTGVLDEQKRDRNWERLQSSMERGGERMPSDWWDLFQAQVRYLAQHRDASGEAWGRMVQAAVRTLWRQLAKQVRDWRKEVRRNADKVKIRGIARDVPGGHSLAQLDYLERQYRFLRSWSAFSVQAGQVVRAERDSRFAVALREHIDNGKKDRLKKLADRILMEALGYVYVTDGRRAGQWQAVYPPCQLVLLEELSEYRFSNDRPPSENSQLMVWSHRGVLEELIHQAQVHDVLVGTIPAAFSSRFDARTGAPGIRCRRVPSIPLKDAPSIPIWLSHYLKQTERDAAALRPGELIPTGDGEFLVTPAGRGASGVRVVHADINAAHNLQRRLWENFDLSDIRVRCDRREGKDGTVVLIPRLTNQRVKERYSGVIFTSEDGVSFTVGDAKTRRRSSASQGEGDDLSDEEQELLAEADDARERSVVLFRDPSGFVNGGRWTAQRAFWGMVHNRIETLLAERFSVSGAAEKVRG

SEQ ID NO: 176 - protein, 1108 aa, unidentified
MATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSM

SEQ ID NO: 177 - protein, 1364 aa, unidentified
MPRDDLDLLTNLNSTAKGIRERGKTKEGTDKKKSGRKSSWPMDKAAWETAKTSDSSAHFLEKLKQHPDLKDAFGNLSSGGSKKLEYYKKLAGSAPWKESQSVILEKAARWKEAKQEREEKEQDSSEHGSKAAYRRLFDAGCLPMPEFAKYIDENQIEFGDLKLSDCGAEWKRGMWNQAGQRVRSHMGWQRRREKENAVYSLRKELFEKGGAIRRKKSEELTPEDILPGKAAPDQNDWQERPAYGNQMWFIGLRSYEENEMAKYAEEAGMGSRSAPRIRRGTIKGWSKLRERWLQILKRNPQATRDDLIGELNALRSQDPRAYGDARLFDWLSKTDQRFLWDGFDADGKILCGRDDRDCVSAFVAYNEEFADEPSSITLTETDERLHPVWPFFGESSAVPYEIEYDLETACPTAIRLPLLVGKENGGYAERQGTRLPLAEYADLASSFQLPTPVRLDVLVEIREVTRAGRKVTCPFSYFKQNGVWYVREGEIPSGESIQIKQTDRKIENGKIFISSKLRMAYRDDLMVSPATGDFGSIKILWERIELASHVDQKKLPETAPARSRVFVSFSCNVVERAPRKQLTRKPDAVVVTIPSGVDQGLVVVSTDVRTGKSKSSSAPPLPPGSRLWPADAVHGDPPLRILSVDLGHRHSAYAVWELGLQQKSWRAGVLKGSTQTPVYADCTGTGLLCLPGDGEDTPAEEESLRLRSRQIRRRLNLQNSILRVSRLLSLDKFEKTIFEQSDVRDRPNKKGLRIRRRCRTEKTPLSEAEVRKNCDKAAEILIRWADTDAMAKSLAATGNADISFWKYMAVKNPPLSAVVDVAPSTIVPDDGPDRETLKKKRQEEEEKFASSIYENRVKLAGALCSGYDADHRRPATGGLWHDLDRTLIREISYGDRGQKGNPRKLNNEGILRLLRRPPRARPDWREFHRTLNDANRIPKGRTLRGGLSMGRLNFLKEVGDFVKKWSCRPRWPGDRRHIPPGQLFDRQDAEHLEHLRDDRIKRLAHLIVAQALGFEPDIRRGLWKYVDGSTGEILWQHPETRRFFAEGAAGELREVSRPAEIDDDAAARPHTVSAPAHIVVFENLIRYRFQSDRPKTENAGLMQWAHRQIVHFTKQVASLYGLKVAMVYAAFSSKFCSRCGSPGARVSRFDPAWRNQEWFKRRTSNPRSKVDHSLKRASEDPTADETRPWVLIEGGKEFVCANAKCSAHDEPLNADENAAANIGLRFLRGVEDFRTKVNPAGALKGKLRFETGIHSFRPPVSGSPFWSPMAEPAQKKKIGAAAPGADVDEAGDADESGVVVLFRDPSGAFRNKQYWYEGKIFWSNVMMAVEAKIAGASVGAKPVAASWGQAQPQSGPGLAKPGGD

SEQ ID NO: 178 - protein, 1364 aa, unidentified
MNRIYQGRVTKVEVPDGKDEKGNIKWKKLENWSDILWQHHMLFQDAVNYYTLALAAISGSAVGSDEKSIILREWAVQVQNIWEKAKKKATVFEGPQKRLTSILGLEQNASFDIAAKHILRTSEAKPEQRASALIRLLEEIDKKNHNVVCGERLPFFCPRNIQSKRSPTSKAVSSVQEQKRQEEVRRFHNMQPEEVVKNAVTLDISLFKSSPKIVFLEDPKKARAELLKQFDNACKKHKELVGIKKAFTESIDKHGSSLKVPAPGSKPSGLYPSAIVFKYFPVDITKTVFLKATEKLAMGKDREVTNDPIADARVNDKPHFDYFTNIALIREKEKNRAAWFEFDLAAFIEAIMSPHRFYQDTQKRKEAARKLEEKIKAIEGKGGQFKESDSEDDDVDSLPGFEGDTRIDLLRKLVTDTLGWLGESETPDNNEGKKTEYSISERTLRIFPDIQKQWSELAEKGETTEGKLLEVLKHEQTEHQSDFGSATLYQHLAKPEFHPIWLKSGTEEWHAENPLKAWLNYKELQYELTDKKRPIHFTPAHPVYSPRYFDFPKKSETEEKEVSKNTHSLTTSLASEHIKNSLQFTAGLIRKTNVGKKAIKARFSYSAPRLRRDCLRSENNENLYKAPWLQPMMRALGIDEEKADRQNFANTRITLMAKGLDDIQLGFPVEANSQELQKEVSNGISWKGQFNWGGIASLSALRWPHEKKPKNPPEQPWWGIDSFSCLAVDLGQRYAGAFARLDVSTIEKKGKSRFIGEACDKKWYAKVSRMGLLRLPGEDVKVWRDASKIDKENGFAFRKELFGEKGRSATPLEAEETAELIKLFGANEKDVMPDNWSKELSFPEQNDKLLIVARRAQAAVSRLHRWAWFFDEAKRSDDAIREILESDDTDLKQKVNKNEIEKVKETIISLLKVKQELLPTLLTRLANRVLPLRGRSWEWKKHHQKNDGFILDQTGKAMPNVLIRGQRGLSMDRIEQITELRKRFQALNQSLRRQIGKKAPAKRDDSIPDCCPDLLEKLDHMKEQRVNQTAHMILAEALGLKLAEPPKDKKELNETCDMHGAYAKVDNPVSFIVIEDLSRYRSSQGRSPRENSRLMKWCHRAVRDKLKEMCEVFFPLCERRKAGSAWVSLPPLLETPAAYSSRFCSRSGVAGFRAVEVIPGFELKYPWSWLKDKKDKAGNLAKEALNIRTVSEQLKAFNQDKPEKPRTLLVPIAGGPIFVPISEVGLSSFGLKPQVVQADINAAINLGLRAISDPRIWEIHPRLRTEKRDGRLFAREKRKYGEEKVEVQPSKNEKAKKVKDDRKPNYFADFSGKVDWGFGNIKNESGLTLVSGKALWWTINQLQWERCFDINKRHIEDWSNKQKQ

SEQ ID NO: 179 - protein, 1413 aa, unidentified
MNRIYQGRVTKVEKLKNGKSPDDREELKDWQTALWRHHELFQDAVSYYTLALAAMAEGLPDKHPINVLRKRMEEAWEEFPRKTVTPAKNLRDSVRPWLGLSESASFGDALKKILPPAPENKEVRALAVALLAEKARTLKPQKTSASYWGRFCDDLKKKPNWDYSEEELARKTGSGDWVAGLWSEDALNKIDELAKSLKLSSLVKCVPDGQINPEGARNLVKEALDHLEGVSNGTKKEKNDPGPAKKTNNWLRQHASDVRNFIHKNKNQFSSLPNGRLITERARGGGININKTYAGVLFKAFPCPFTFDYVRAAVPEPKVKKVDQEKKSEQSATWTELEKRILRIGDDPIELARKNNKPIFKAFTALEKWSDQNSKSCWSDFDKCAFEEALKTLNQFNQKTEEREKRRSEAEAELKYMMDENPEWKPKKETEGDDVREVPILKGDPRYEKLVKLFGDLDEEGSEHATGKIYGPSRASLRGFGKLRNEWVDLFTKANDNPREQDLQKAVTGFQREHKLDMGYTAFFLKLCERDYWDIWRDDTEVEVKKIREKRWVKSVVYAAADTRELAEELERLQEPVRYTPAEPQFSRRLFMFSDIKGKQGAKHIREGLVEVSLAVKDQSGKYGTCRVRLHYSAPRLIRDHLSDGSSSMWLQPMMAALGLSSDARGCFTRDSKGNVKEPAVALMSDFVGRKRELRMLLNFPVDLDISKLEENIGKKARWEKQMNTAYEKNKLKQRFHLIWPGMELKETQEPGQFWWDNPTIQKEGMYCLAIDLSQRRAADYALLHAGVNRDSKTFVELGQAGGQSWFTKLCAAGSLRLPGEDTEVIREGKRQIELSGKKGRNATQSEYDQAIALAKQLLHNENSAELESAARDWLGDNAKRFSFPEQNDKLIDLYYGALSRYKTWLRWSWRLTEQHKELWDKTLDEIRKVPYFASWGELAGNGTNEATVQQLQKLIADAAVDLRNFLEKALLHIAYRALPLRENTWRWIENGKDGKGKPLHLLVSDGQSPAEIPWLRGQRGLSIARIEQLENFRRAVLSLNRLLRHEIGTKPEFGSSTCGESLPDPCPDLTDKIVRLKEERVNQTAHLIIAQSLGVRLKGHSLFTEEREKADMHGEHEVIPGRSPVDFVVLEDLSRYTTDKSRSRSENSRLMKWCHRKINEKVKLLAEPFGIPVIEVFASYSSKFDARTGAPGFRAVEVTSEDRPFWRKTIEKQSVAREVFDCLDNLVGKGLNGIHLVLPQNGGPLFIAAVKEDQPLPAIRQADINAAVNIGLRAIAGPSCYHAHPKVRLIKGESGTDKGKWLPRKGKEANKRENAQFGNVDLDLEVKFNRLDIDSDVLKGDNTNLFHDPLNIACYGFATIQNLQHPFLAHASAVFSRQKGAVARLQWEVCRAINSRRLEAWQKKAEKAAVKR

SEQ ID NO: 180 - protein, 747 aa, unidentified
MATKSYRARILTDSRLAAALDRTHVVFVESLKQMINTYLRMQNGKFGPDHKKLAQIMLSRSNTFAHGVMDQITRDQPTSTLDEEWTDLARRIHKTTGPLFLQAERFATVKNRAIHTKSRGKVIPSPETLAVPAKFWHQVCDSASAYIRSNRELMQQWRKDRAAWLKDKNEWQQKHPEFMQFYNGPYQNFLKLCDDDRITSQLAAEQQPTASKNNRPRKTGKRFARWHLWYKWLSENPEIIEWRNKASASDFKTVTDDVRKQIITKYPQQNKYITRLLDWLEDNNPELKTLENLRRTYVKKFDSFKRPPTLTLPSPYRHPYWFTMELDQFYKKADFENGTIQLLLIDEDDDGNWFFNWMPASLKPDPRLVPSWRAETFETEGRFPPYLGGKIGKKLSRPAPTDAERKAGIAGAKLMIKNNRSELLFTVFEQDCPPRVKWAKTKNRKCPADNAFSSDGKTRKPLRILSIDLGIRHIGAFALTQGTRNDSAWQTESLKKGIINSPSIPPLRQVRRHDYDLKRKRRRHGKPVKGQRSNANLQAHRTNMAQDRFKKGASAIVSLAREHSADLILFENLHSLKFSAFDERWMNRQLRDMNRRHIVELVSEQAPEFGITVKDDINPWMTSRICSNCNLPGFRFSMKKKNPYREKLPREKCTDFGYPVWEPGGHLFRCPHCDHRVNADINAAANLANKFFGLGYWNNGLKYDAETKTFTVHTDKKTPPLIFKPRPQFDLWADSVKTRKQLGPDPF

SEQ ID NO: 181 - protein, 743 aa, unidentified
MSVRSFQARVECDKQTMEHLWRTHKVFNERLPEIIKILFKMKRGECGQNDKQKSLYKSISQSILEANAQNADYLLNSVSIKGWKPGTAKKYRNASFTWADDAAKLSSQGIHVYDKKQVLGDLPGMMSQMVCRQSVEAISGHIELTKKWEKEHNEWLKEKEKWESEDEHKKYLDLREKFEQFEQSIGGKITKRRGRWHLYLKWLSDNPDFAAWRGNKAVINPLSEKAQIRINKAKPNKKNSVERDEFFKANPEMKALDNLHGYYERNFVRRRKTKKNPDGFDHKPTFTLPHPTIHPRWFVFNKPKTNPEGYRKLILPKKAGDLGSLEMRLLTGEKNKGNYPDDWISVKFKADPRLSLIRPVKGRRVVRKGKEQGQTKETDSYEFFDKHLKKWRPAKLSGVKLIFPDKTPKAAYLYFTCDIPDEPLTETAKKIQWLETGDVTKKGKKRKKKVLPHGLVSCAVDLSMRRGTTGFATLCRYENGKIHILRSRNLWVGYKEGKGCHPYRWTEGPDLGHIAKHKREIRILRSKRGKPVKGEESHIDLQKHIDYMGEDRFKKAARTIVNFALNTENAASKNGFYPRADVLLLENLEGLIPDAEKERGINRALAGWNRRHLVERVIEMAKDAGFKRRVFEIPPYGTSQVCSKCGALGRRYSIIRENNRREIRFGYVEKLFACPNCGYCANADHNASVNLNRRFLIEDSFKSYYDWKRLSEKKQKEEIETIESKLMDKLCAMHKISRGSISK

SEQ ID NO: 182 - protein, 1119 aa, unidentified
MSFTISYPFKLIIKNKDEAKALLDTHQYMNEGVKYYLEKLLMFRQEKIFIGEDETGKRIYIEETEYKKQIEEFYLIKKTELGRNLTLTLDEFKTLMRELYICLVSSSMENKKGFPNAQQASLNIFSPLFDAESKGYILKEENNNISLIHKDYGKILLKRLRDNNLIPIFTKFTDIKKITAKLSPTALDRMIFAQAIEKLLSYESWCKLMIKERFDKEVKIKELENKCENKQERDKIFEILEKYEEERQKTFEQDSGFAKKGKFYITGRMLKGFDEIKEKWLKEKDRSEQNLINILNKYQTDNSKLVGDRNLFEFIIKLENQCLWNGDIDYLKIKRDINKNQIWLDRPEMPRFTMPDFKKHPLWYRYEDPSNSNFRNYKIEVVKDENYITIPLITERNNEYFEENYTFNLAKLKKLSENITFIPKSKNKEFEFIDSNDEEEDKKDQKKSKQYIKYCDTAKNTSYGKSGGIRLYFNRNELENYKDGKKMDSYTVFTLSIRDYKSLFAKEKLQPQIFNTVDNKITSLKIQKKFGNEEQTNFLSYFTQNQITKKDWMDEKTFQNVKELNEGIRVLSVDLGQRFFAAVSCFEIMSEIDNNKLFFNLNDQNHKIIRINDKNYYAKHIYSKTIKLSGEDDDLYKERKINKNYKLSYQERKNKIGIFTRQINKLNQLLKIIRNDEIDKEKFKELIETTKRYVKNTYNDGIIDWNNVDNKILSYENKEDVINLHKELDKKLEIDFKEFIRECRKPIFRSGGLSMQRIDFLEKLNKLKRKWVARTQKSAESIVLTPKFGYKLKEHINELKDNRVKQGVNYILMTALGYIKDNEIKNDSKKKQKEDWVKKNRACQIILMEKLTEYTFAEDRPREENSKLRMWSHRQIFNFLQQKASLWGILVGDVFAPYTSKCLSDNNAPGIRCHQVTKKDLIDNSWFLKIVVKDDAFCDLIEINKENVKNKSIKINDILPLRGGELFASIKDGKLHIVQADINASRNIAKRFLSQINPFRVVLKKDKDETFHLKNEPNYLKNYYSILNFVPTNEELTFFKVEENKDIKPTKRIKMDKHEKESTDEGDDYSKNQIALFRDDSGIFFDKSLWVDGKIFWSVVKNKMTKLLRERNNKKNGSK

SEQ ID NO: 183 - protein, 1447 aa, unidentified
MPLSRIYQGRTNSLIILTPTPQEPWDHKALARFDSPLWRHHALFQDAVNYYQLCLVALASSDGTRPLSKLHEQMKASWDEAKTDTEDSWRVRLARRLGIPAASLFEAALAKVLEGNEAPERARELAGELLLDKIEGDIQQAGRGYWPRFCDPKANPTYDYSATARASASGLTKLAAVIHAENVTEEALKQVAAEMDLSWTVKLQPDKNFVGAEARARLLEAAHHFIKVAESPPTKLAEVLARFPDGLALWQALPEKIAALPEETQVPRNRKASPDLTFATLLFQHFPSLFTAAVLGLSVGKPKSVKAPKVVEKVSARRKANAVTQAVVIEEPEIDFAELGDDPIKLARGERGFVFPAFTSLSFWAVPGPHVPVWKEFDIAAFKEALKTVNQFKLKTSERNALLAEAQRRLDYMDEKTHDWKTGDSDEPGHIPPRLKSDPNFTLIQALTQDEGVSNKATGDQHIPKGVYTGGLRGFYAIKKDWCELWERKADKSQGTPTEEELISIVTDYQRDHVYDVGDVGLFRALCEPRFWPLWQPLTDEQEAERIKAGRAKDMISAYRVWLELQEDVVRLAQPIRFTPAHAENSRRLFMFSDISGSHGAEFGSDGKSLEVSIAYDVDGKLQPVRAKLEFSAPRAARDELEGLSGGSESMRWFQPMMKALDCPEVEMPALEKCAVSLMPDVVKKGGGKWVRLLLNFPATLEPEGLIRHIGKQAMWYKQFNGTYKPRTQQLDTGLHLYWPGLEKAPEAEDAAAWWNREEIRAKGFSVLSVDLGQRDAGAWALLESRSDKAFSRNRQPFIELGEAGGKLWSTALLGLGMLRLPGEDARTGALDDQGKRAVEFHGKAGRNALEAEWQEAREMALLFGGEEAKSRLGPGFDHLSHSKQNEELLRILSRAQSRLARFHRWSCRIHEKPEATGDDVIDYGQVDELLTKTAEAMLENLKALYTNAGGILDSKSKQPLTLVGLRKKLEAQKVEPEKIAAVLKPHAEIIFQRLGTLIPELKQHLRVSLERLANRELPLRHREWVWNEAFEKLEQGNFKKEENPKWIRGQRGLSMARIEQIENLRKRFMSLRRQMSLIPGEQVKQGVEDKGQRQPEPCEDILNKLDRMKQQRVNQTAHLILAQALGLRLRPHLANDAEREEKDIHGEYELIPGRKPVDFIVMEDLSRYLSSQGRAPSENGRLMKWCHRAVLAKLKQMCEPFGIPVLEVPAAYSSRFCALTGVPGFRAVEVHDGNAEDFRWKRLIKKAEKDKSSKDAEAAAMLFDQLHDLNIEAREARKQDKKLPLRTLFAPVAGGPLFIPMVGGGPRQADMNAAINLGLRAIASPTCLRARPKIRAELKDGKHQAMLGNKLEKAAALTLEPPKEPTKELAAQKRTNFFLDEKFVGKFDTAHVTTSGKKLRLSGGMSLWKAIKDGAWQRVKKINDARIAKWKNNPPPEPDPDDEIQF

SEQ ID NO: 184 - protein, 1147 aa, Alicyclobacillus kakegawensis
MAVKSIKVKLRLSECPDILAGMWQLHRATNAGVRYYTEWVSLMRQEILYSRGPDGGQQCYMTAEDCQRELLRRLRNRQLHNGRQDQPGTDADLLAISRRLYEILVLQSIGKRGDAQQIASSFLSPLVDPNSKGGRGEAKSGRKPAWQKMRDQGDPRWVAAREKYEQRKAVDPSKEILNSLDALGLRPLFAVFTETYRSGVDWKPLGKSQGVRTWDRDMFQQALERLMSWESWNRRVGEEYARLFQQKMKFEQEHFAEQSHLVKLARALEADMRAASQGFEAKRGTAHQITRRALRGADRVFEIWKSIPEEALFSQYDEVIRQVQAEKRRDFGSHDLFAKLAEPKYQPLWRADETFLTRYALYNGVLRDLEKARQFATFTLPDACVNPIWTRFESSQGSNLHKYEFLFDHLGPGRHAVRFQRLLVVESEGAKERDSVVVPVAPSGQLDKLVLREEEKSSVALHLHDTARPDGFMAEWAGAKLQYERSTLARKARRDKQGMRSWRRQPSMLMSAAQMLEDAKQAGDVYLNISVRVKSPSEVRGQRRPPYAALFRIDDKQRRVTVNYNKLSAYLEEHPDKQIPGAPGLLSGLRVMSVDLGLRTSASISVFRVAKKEEVEALGDGRPPHYYPIHGTDDLVAVHERSHLIQMPGETETKQLRKLREERQAVLRPLFAQLALLRLLVRCGAADERIRTRSWQRLTKQGREFTKRLTPSWREALELELTRLEAYCGRVPDDEWSRIVDRTVIALWRRMGKQVRDWRKQVKSGAKVKVKGYQLDVVGGNSLAQIDYLEQQYKFLRRWSFFARASGLVVRADRESHFAVALRQHIENAKRDRLKKLADRILMEALGYVYEASGPREGQWTAQHPPCQLIILEELSAYRFSDDRPPSENSKLMAWGHRGILEELVNQAQVHDVLVGTVYAAFSSRFDARTGAPGVRCRRVPARFVGATVDDSLPLWLTEFLDKHRLDKNLLRPDDVIPTGEGEFLVSPCGEEAARVRQVHADINAAQNLQRRLWQNFDITELRLRCDVKMGGEGTVLVPRVNNARAKQLFGKKVLVSQDGVTFFERSQTGGKPHSEKQTDLTDKELELIAEADEARAKSVVLFRDPSGHIGKGHWIRQREFWSLVKQRIESHTAERIRVRGVGSSLD

SEQ ID NO: 185 - protein, 1112 aa, Bacillus sp.
MAIRSIKLKMKTNSGTDSIYLRKALWRTHQLINEGIAYYMNLLTLYRQEAIGDKTKEAYQAELINIIRNQQRNNGSSEEHGSDQEILALLRQLYELIIPSSIGESGDANQLGNKFLYPLVDPNSQSGKGTSNAGRKPRWKRLKEEGNPDWELEKKKDEERKAKDPTVKIFDNLNKYGLLPLFPLFTNIQKDIEWLPLGKRQSVRKWDKDMFIQAIERLLSWESWNRRVADEYKQLKEKTESYYKEHLTGGEEWIEKIRKFEKERNMELEKNAFAPNDGYFITSRQIRGWDRVYEKWSKLPESASPEELWKVVAEQQNKMSEGFGDPKVFSFLANRENRDIWRGHSERIYHIAAYNGLQKKLSRTKEQATFTLPDAIEHPLWIRYESPGGTNLNLFKLEEKQKKNYYVTLSKIIWPSEEKWIEKENIEIPLAPSIQFNRQIKLKQHVKGKQEISFSDYSSRISLDGVLGGSRIQFNRKYIKNHKELLGEGDIGPVFFNLVVDVAPLQETRNGRLQSPIGKALKVISSDFSKVIDYKPKELMDWMNTGSASNSFGVASLLEGMRVMSIDMGQRTSASVSIFEVVKELPKDQEQKLFYSINDTELFAIHKRSFLLNLPGEVVTKNNKQQRQERRKKRQFVRSQIRMLANVLRLETKKTPDERKKAIHKLMEIVQSYDSWTASQKEVWEKELNLLTNMAAFNDEIWKESLVELHHRIEPYVGQIVSKWRKGLSEGRKNLAGISMWNIDELEDTRRLLISWSKRSRTPGEANRIETDEPFGSSLLQHIQNVKDDRLKQMANLIIMTALGFKYDKEEKDRYKRWKETYPACQIILFENLNRYLFNLDRSRRENSRLMKWAHRSIPRTVSMQGEMFGLQVGDVRSEYSSRFHAKTGAPGIRCHALTEEDLKAGSNTLKRLIEDGFINESELAYLKKGDIIPSQGGELFVTLSKRYKKDSDNNELTVIHADINAAQNLQKRFWQQNSEVYRVPCQLARMGEDKLYIPKSQTETIKKYFGKGSFVKNNTEQEVYKWEKSEKMKIKTDTTFDLQDLDGFEDISKTIELAQEQQKKYLTMFRDPSGYFFNNETWRPQKEYWSIVNNIIKSCLKKKILSNKVEL

SEQ ID NO: 186 - protein, 1489 aa, Desulfatirhabdium butyrativorans
MPLSNNPPVTQRAYTLRLRGADPSDLSWREALWHTHEAVNKGAKVFGDWLLTLRGGLDHTLADTKVKGGKGKPDRDPTPEERKARRILLALSWLSVESKLGAPSSYIVASGDEPAKDRNDNVVSALEEILQSRKVAKSEIDDWKRDCSASLSAAIRDDAVWVNRSKVFDEAVKSVGSSLTREEAWDMLERFFGSRDAYLTPMKDPEDKSSETEQEDKAKDLVQKAGQWLSSRYGTSEGADFCRMSDIYGKIAAWADNASQGGSSTVDDLVSELRQHFDTKESKATNGLDWIIGLSSYTGHTPNPVHELLRQNTSLNKSHLDDLKKKANTRAESCKSKIGSKGQRPYSDAILNDVESVCGFTYRVDKDGQPVSVADYSKYDVDYKWGTARHYIFAVMLDHAARRISLAHKWIKRAEAERHKFEEDAKRIANVPARAREWLDSFCKERSVTSGAVEPYRIRRRAVDGWKEVVAAWSKSDCKSTEDRIAAARALQDDSEIDKFGDIQLFEALAEDDALCVWHKDGEATNEPDFQPLIDYSLAIEAEFKKRQFKVPAYRHPDELLHPVFCDFGKSRWKINYDVHKNVQAPFYRGLCLTLWTGSEIKPVPLCWQSKRLTRDLALGNNHRNDAASAVTRADRLGRAASNVTKSDMVNITGLFEQADWNGRLQAPRQQLEAIAVVRDNPRLSEQERNLRMCGMIEHIRWLVTFSVKLQPQGPWCAYAEQHGLNTNPQYWPHADTNRDRKVHARLILPRLPGLRVLSVDLGHRYAAACAVWEAVNTETVKEACQNVGRDMPKEHDLYLHIKVKKQGIGKQTEVDKTTIYRRIGADTLPDGRPHPAPWARLDRQFLIKLQGEEKDAREASNEEIWALHQMECKLDRTKPLIDRLIASGWGLLKRQMARLDALKELGWIPAPDSSENLSREDGEAKDYRESLAVDDLMFSAVRTLRLALQRHGNRARIAYYLISEVKIRPGGIQEKLDENGRIDLLQDALALWHELFSSPGWRDEAAKQLWDSRIATLAGYKAPEENGDNVSDVAYRKKQQVYREQLRNVAKTLSGDVITCKELSDAWKERWEDEDQRWKKLLRWFKDWVLPSGTQANNATIRNVGGLSLSRLATITEFRRKVQVGFFTRLRPDGTRHEIGEQFGQKTLDALELLREQRVKQLASRIAEAALGIGSEGGKGWDGGKRPRQRINDSRFAPCHAVVIENLANYRPDETRTRLENRRLMTWSASKVHKYLSEACQLNGLYLCTVSAWYTSRQDSRTGAPGIRCQDVSVREFMQSPFWRKQVKQAEAKHDENKGDARERFLCELNKTWKAKTPAEWKKAGFVRIPLRGGEIFVSADSKSPSAKGIHADLNAAANIGLRALTDPDWPGKWWYVPCDPVSFESKMDYVKGCAAVKVGQPLRQPAQTNADGAASKIRKGKKNRTAGTSKEKVYLWRDISAFPLESNEIGEWKETSAYQNDVQYRVIRMLKEHIKSLDNRTGDNVEG

SEQ ID NO: 187 - protein, 1194 aa, Desulfonatronum thiodismutans
MVLGRKDDTAELRRALWTTHEHVNLAVAEVERVLLRCRGRSYWTLDRRGDPVHVPESQVAEDALAMAREAQRRNGWPVVGEDEEILLALRYLYEQIVPSCLLDDLGKPLKGDAQKIGTNYAGPLFDSDTCRRDEGKDVACCGPFHEVAGKYLGALPEWATPISKQEFDGKDASHLRFKATGGDDAFFRVSIEKANAWYEDPANQDALKNKAYNKDDWKKEKDKGISSWAVKYIQKQLQLGQDPRTEVRRKLWLELGLLPLFIPVFDKTMVGNLWNRLAVRLALAHLLSWESWNHRAVQDQALARAKRDELAALFLGMEDGFAGLREYELRRNESIKQHAFEPVDRPYVVSGRALRSWTRVREEWLRHGDTQESRKNICNRLQDRLRGKFGDPDVFHWLAEDGQEALWKERDCVTSFSLLNDADGLLEKRKGYALMTFADARLHPRWAMYEAPGGSNLRTYQIRKTENGLWADVVLLSPRNESAAVEEKTFNVRLAPSGQLSNVSFDQIQKGSKMVGRCRYQSANQQFEGLLGGAEILFDRKRIANEQHGATDLASKPGHVWFKLTLDVRPQAPQGWLDGKGRPALPPEAKHFKTALSNKSKFADQVRPGLRVLSVDLGVRSFAACSVFELVRGGPDQGTYFPAADGRTVDDPEKLWAKHERSFKITLPGENPSRKEEIARRAAMEELRSLNGDIRRLKAILRLSVLQEDDPRTEHLRLFMEAIVDDPAKSALNAELFKGFGDDRFRSTPDLWKQHCHFFHDKAEKVVAERFSRWRTETRPKSSSWQDWRERRGYAGGKSYWAVTYLEAVRGLILRWNMRGRTYGEVNRQDKKQFGTVASALLHHINQLKEDRIKTGADMIIQAARGFVPRKNGAGWVQVHEPCRLILFEDLARYRFRTDRSRRENSRLMRWSHREIVNEVGMQGELYGLHVDTTEAGFSSRYLASSGAPGVRCRHLVEEDFHDGLPGMHLVGELDWLLPKDKDRTANEARRLLGGMVRPGMLVPWDGGELFATLNAASQLHVIHADINAAQNLQRRFWGRCGEAIRIVCNQLSVDGSTRYEMAKAPKARLLGALQQLKNGDAPFHLTSIPNSQKPENSYVMTPTNAGKKYRAGPGEKSSGEEDELALDIVEQAEELAQGRKTFFRDPSGVFFAPDRWLPSEIYWSRIRRRIWQVTLERNSSGRQERAEMDEMPY

SEQ ID NO: 188 - protein, 1326 aa, unidentified
MAVELNRIYQGRVNHVYIFDENQNQVSVDNGDDLLFVHHELYQDAINYYLVALAAMALDSKDSLFGKFKMQIRAVWNDFYRNGQLRPGLKHSLIRSLGHAAELNTSNGADIAMNLILEDGGIPSEILNAALEHLAEKCTGDVSQLGKTFFPRFCDTAYHGNWDVDAKSFSEKKGRQRLVDALYSLHPVQAVQELAPEIEIGWGGVKTQTGKFFTGDEAKASLKKAISYFLQDTGKNSPELQEYFSVAGKQPLEQYLGKIDTFPEISFGRISSHQNINISNAMWILKFFPDQYSVDLIKNLIPNKKYEIGIAPQWGDDPVKLSRGKRGYTFRAFTDLAMWEKNWKVFDRAAFSDALKTINQFRNKTQERNDQLKRYCAALNWMDGESSDKKPPVEPADADAVDEAATSVLPILAGDKRWNALLQLQKELGICNDFTENELMDYGLSLRTIRGYQKLRSMMLEKEEKMRAKTADDEEISQALQEIIIKFQSSHRDTIGSVSLFLKLAEPKYFCVWHDADKNQNFASVDMVADAVRYYSYQEEKARLEEPIQITPADARYSRRVSDLYALVYKNAKECKTGYGLRPDGNFVFEIAQKNAKGYAPAKVVLAFSAPRLKRDGLIDKEFSAYYPPVLQAFLREEEAPKQSFKTTAVILMPDWDKNGKRRILLNFPIKLDVSAIHQKTDHRFENQFYFANNTNTCLLWPSYQYKKPVTWYQGKKPFDVVAVDLGQRSAGAVSRITVSTEKREHSVAIGEAGGTQWYAYRKFSGLLRLPGEDATVIRDGQRTEELSGNAGRLSTEEETVQACVLCKMLIGDATLLGGSDEKTIRSFPKQNDKLLIAFRRATGRMKQLQRWLWMLNENGLCDKAKTEISNSDWLVNKNIDNVLKEEKQHREMLPAILLQIADRVLPLRGRKWDWVLNPQSNSFVLQQTAHGSGDPHKKICGQRGLSFARIEQLESLRMRCQALNRILMRKTGEKPATLAEMRNNPIPDCCPDILMRLDAMKEQRINQTANLILAQALGLRHCLHSESATKRKENGMHGEYEKIPGVEPAAFVVLEDLSRYRFSQDRSSYENSRLMKWSHRKILEKLALLCEVFNVPILQVGAAYSSKFSANAIPGFRAEECSIDQLSFYPWRELKDSREKALVEQIRKIGHRLLTFDAKATIIMPRNGGPVFIPFVPSDSKDTLIQADINASFNIGLRGVADATNLLCNNRVSCDRKKDCWQVKRSSNFSKMVYPEKLSLSFDPIKKQEGAGGNFFVLGCSERILTGTSEKSPVFTSSEMAKKYPNLMFGSALWRNEILKLERCCKINQSRLDKFIAKKEVQNEL

SEQ ID NO: 189 - protein, 1090 aa, Laceyella sediminis
MSIRSFKLKIKTKSGVNAEELRRGLWRTHQLINDGIAYYMNWLVLLRQEDLFIRNEETNEIEKRSKEEIQGELLERVHKQQQRNQWSGEVDDQTLLQTLRHLYEEIVPSVIGKSGNASLKARFFLGPLVDPNNKTTKDVSKSGPTPKWKKMKDAGDPNWVQEYEKYMAERQTLVRLEEMGLIPLFPMYTDEVGDIHWLPQASGYTRTWDRDMFQQAIERLLSWESWNRRVRERRAQFEKKTHDFASRFSESDVQWMNKLREYEAQQEKSLEENAFAPNEPYALTKKALRGWERVYHSWMRLDSAASEEAYWQEVATCQTAMRGEFGDPAIYQFLAQKENHDIWRGYPERVIDFAELNHLQRELRRAKEDATFTLPDSVDHPLWVRYEAPGGTNIHGYDLVQDTKRNLTLILDKFILPDENGSWHEVKKVPFSLAKSKQFHRQVWLQEEQKQKKREVVFYDYSTNLPHLGTLAGAKLQWDRNFLNKRTQQQIEETGEIGKVFFNISVDVRPAVEVKNGRLQNGLGKALTVLTHPDGTKIVTGWKAEQLEKWVGESGRVSSLGLDSLSEGLRVMSIDLGQRTSATVSVFEITKEAPDNPYKFFYQLEGTELFAVHQRSFLLALPGENPPQKIKQMREIRWKERNRIKQQVDQLSAILRLHKKVNEDERIQAIDKLLQKVASWQLNEEIATAWNQALSQLYSKAKENDLQWNQAIKNAHHQLEPVVGKQISLWRKDLSTGRQGIAGLSLWSIEELEATKKLLTRWSKRSREPGVVKRIERFETFAKQIQHHINQVKENRLKQLANLIVMTALGYKYDQEQKKWIEVYPACQVVLFENLRSYRFSYERSRRENKKLMEWSHRSIPKLVQMQGELFGLQVADVYAAYSSRYHGRTGAPGIRCHALTEADLRNETNIIHELIEAGFIKEEHRPYLQQGDLVPWSGGELFATLQKPYDNPRILTLHADINAAQNIQKRFWHPSMWFRVNCESVMEGEIVTYVPKNKTVHKKQGKTFRFVKVEGSDVYEWAKWSKNRNKNTFSSITERKPPSSMILFRDPSGTFFKEQEWVEQKTFWGKVQSMIQAYMKKTIVQRMEE

SEQ ID NO: 190 - protein, 1041 aa, Methylobacterium nodulans
MYEAIVLADDANAQLANAFLGPLTDPNSAGFLEAFNKVDRPAPSWLDQVPASDPIDPAVLAEANAWLDTDAGRAWLVDTGAPPRWRSLAAKQDPIWPREFARKLGELRKEAASGTSAIIKALKRDFGVLPLFQPSLAPRILGSRSSLTPWDRLAFRLAVGHLLSWESWCTRARDEHTARVQRLEQFSSAHLKGDLATKVSTLREYERARKEQIAQLGLPMGERDFLITVRMTRGWDDLREKWRRSGDKGQEALHAIIATEQTRKRGRFGDPDLFRWLARPENHHVWADGHADAVGVLARVNAMERLVERSRDTALMTLPDPVAHPRSAQWEAEGGSNLRNYQLEAVGGELQITLPLLKAADDGRCIDTPLSFSLAPSDQLQGVVLTKQDKQQKITYCTNMNEVFEAKLGSADLLLNWDHLRGRIRDRVDAGDIGSAFLKLALDVAHVLPDGVDDQLARAAFHFQSAKGAKSKHADSVQAGLRVLSIDLGVRSFATCSVFELKDTAPTTGVAFPLAEFRLWAVHERSFTLELPGENVGAAGQQWRAQADAELRQLRGGLNRHRQLLRAATVQKGERDAYLTDLREAWSAKELWPFEASLLSELERCSTVADPLWQDTCKRAARLYRTEFGAVVSEWRSRTRSREDRKYAGKSMWSVQHLTDVRRFLQSWSLAGRASGDIRRLDRERGGVFAKDLLDHIDALKDDRLKTGADLIVQAARGFQRNEFGYWVQKHAPCHVILFEDLSRYRMRTDRPRRENSQLMQWAHRGVPDMVGMQGEIYGIQDRRDPDSARKHARQPLAAFCLDTPAAFSSRYHASTMTPGIRCHPLRKREFEDQGFLELLKRENEGLDLNGYKPGDLVPLPGGEVFVCLNANGLSRIHADINAAQNLQRRFWTQHGDAFRLPCGKSAVQGQIRWAPLSMGKRQAGALGGFGYLEPTGHDSGSCQWRKTTEAEWRRLSGAQKDRDEAAAAEDEELQGLEEELLERSGERVVFFRDPSGVVLPTDLWFPSAAFWSIVRAKTVGRLRSHLDAQAEASYAVAAGL

SEQ ID NO: 191 - protein, 1388 aa, unidentified
MSLNRIYQGRVAAVETGTALAKGNVEWMPAAGGDEVLWQHHELFQAAINYYLVALLALADKNNPVLGPLISQMDNPQSPYHVWGSFRRQGRQRTGLSQAVAPYITPGNNAPTLDEVFRSILAGNPTDRATLDAALMQLLKACDGAGAIQQEGRSYWPKFCDPDSTANFAGDPAMLRREQHRLLLPQVLHDPAITHDSPALGSFDTYSIATPDTRTPQLTGPKARARLEQAITLWRVRLPESAADFDRLASSLKKIPDDDSRLNLQGYVGSSAKGEVQARLFALLLFRHLERSSFTLGLLRSATPPPKNAETPPPAGVPLPAASAADPVRIARGKRSFVFRAFTSLPCWHGGDNIHPTWKSFDIAAFKYALTVINQIEEKTKERQKECAELETDFDYMHGRLAKIPVKYTTGEAEPPPILANDLRIPLLRELLQNIKVDTALTDGEAVSYGLQRRTIRGFRELRRIWRGHAPAGTVFSSELKEKLAGELRQFQTDNSTTIGSVQLFNELIQNPKYWPIWQAPDVETARQWADAGFADDPLAALVQEAELQEDIDALKAPVKLTPADPEYSRRQYDFNAVSKFGAGSRSANRHEPGQTERGHNTFTTEIAARNAADGNRWRATHVRIHYSAPRLLRDGLRRPDTDGNEALEAVPWLQPMMEALAPLPTLPQDLTGMPVFLMPDVTLSGERRILLNLPVTLEPAALVEQLGNAGRWQNQFFGSREDPFALRWPADGAVKTAKGKTHIPWHQDRDHFTVLGVDLGTRDAGALALLNVTAQKPAKPVHRIIGEADGRTWYASLADARMIRLPGEDARLFVRGKLVQEPYGERGRNASLLEWEDARNIILRLGQNPDELLGADPRRHSYPEINDKLLVALRRAQARLARLQNRSWRLRDLAESDKALDEIHAERAGEKPSPLPPLARDDAIKSTDEALLSQRDIIRRSFVQIANLILPLRGRRWEWRPHVEVPDCHILAQSDPGTDDTKRLVAGQRGISHERIEQIEELRRRCQSLNRALRHKPGERPVLGRPAKGEEIADPCPALLEKINRLRDQRVDQTAHAILAAALGVRLRAPSKDRAERRHRDIHGEYERFRAPADFVVIENLSRYLSSQDRARSENTRLMQWCHRQIVQKLRQLCETYGIPVLAVPAAYSSRFSSRDGSAGFRAVHLTPDHRHRMPWSRILARLKAHEEDGKRLEKTVLDEARAVRGLFDRLDRFNAGHVPGKPWRTLLAPLPGGPVFVPLGDATPMQADLNAAINIALRGIAAPDRHDIHHRLRAENKKRILSLRLGTQREKARWPGGAPAVTLSTPNNGASPEDSDALPERVSNLFVDIAGVANFERVTIEGVSQKFATGRGLWASVKQRAWNRVARLNETVTDNNRNEEEDDIPM

SEQ ID NO: 192 - protein, 1465 aa, Thermomonas hydrothermalis
MSEKTTQRAYTLRLNRASGECAVCQNNSCDCWHDALWATHKAVNRGAKAFGDWLLTLRGGLCHTLVEMEVPAKGNNPPQRPTDQERRDRRVLLALSWLSVEDEHGAPKEFIVATGRDSADDRAKKVEEKLREILEKRDFQEHEIDAWLQDCGPSLKAHIREDAVWVNRRALFDAAVERIKTLTWEEAWDFLEPFFGTQYFAGIGDGKDKDDAEGPARQGEKAKDLVQKAGQWLSARFGIGTGADFMSMAEAYEKIAKWASQAQNGDNGKATIEKLACALRPSEPPTLDTVLKCISGPGHKSATREYLKTLDKKSTVTQEDLNQLRKLADEDARNCRKKVGKKGKKPWADEVLKDVENSCELTYLQDNSPARHREFSVMLDHAARRVSMAHSWIKKAEQRRRQFESDAQKLKNLQERAPSAVEWLDRFCESRSMTTGANTGSGYRIRKRAIEGWSYVVQAWAEASCDTEDKRIAAARKVQADPEIEKFGDIQLFEALAADEAICVWRDQEGTQNPSILIDYVTGKTAEHNQKRFKVPAYRHPDELRHPVFCDFGNSRWSIQFAIHKEIRDRDKGAKQDTRQLQNRHGLKMRLWNGRSMTDVNLHWSSKRLTADLALDQNPNPNPTEVTRADRLGRAASSAFDHVKIKNVFNEKEWNGRLQAPRAELDRIAKLEEQGKTEQAEKLRKRLRWYVSFSPCLSPSGPFIVYAGQHNIQPKRSGQYAPHAQANKGRARLAQLILSRLPDLRILSVDLGHRFAAACAVWETLSSDAFRREIQGLNVLAGGSGEGDLFLHVEMTGDDGKRRTVVYRRIGPDQLLDNTPHPAPWARLDRQFLIKLQGEDEGVREASNEELWTVHKLEVEVGRTVPLIDRMVRSGFGKTEKQKERLKKLRELGWISAMPNEPSAETDEKEGEIRSISRSVDELMSSALGTLRLALKRHGNRARIAFAMTADYKPMPGGQKYYFHEAKEASKNDDETKRRDNQIEFLQDALSLWHDLFSSPDWEDNEAKKLWQNHIATLPNYQTPEEISAELKRVERNKKRKENRDKLRTAAKALAENDQLRQHLHDTWKERWESDDQQWKERLRSLKDWIFPRGKAEDNPSIRHVGGLSITRINTISGLYQILKAFKMRPEPDDLRKNIPQKGDDELENFNRRLLEARDRLREQRVKQLASRIIEAALGVGRIKIPKNGKLPKRPRTTVDTPCHAVVIESLKTYRPDDLRTRRENRQLMQWSSAKVRKYLKEGCELYGLHFLEVPANYTSRQCSRTGLPGIRCDDVPTGDFLKAPWWRRAINTAREKNGGDAKDRFLVDLYDHLNNLQSKGEALPATVRVPRQGGNLFIAGAQLDDTNKERRAIQADLNAAANIGLRALLDPDWRGRWWYVPCKDGTSEPALDRIEGSTAFNDVRSLPTGDNSSRRAPREIENLWRDPSGDSLESGTWSPTRAYWDTVQSRVIELLRRHAGLPTS

SEQ ID NO: 193 - protein, 370 aa, Methylobacterium nodulans
MYEAIVLADDANAQLANAFLGPLTDPNSAGFLEAFNKVDRPAPSWLDQVPASDPIDPAVLAEANAWLDTDAGRAWLVDTGAPPRWRSLAAKQDPIWPREFARKLGELRKEAASGTSAIIKALKRDFGVLPLFQPSLAPRILGSRSSLTPWDRLAFRLAVGHLLSWESWCTRARDEHTARVQRLEQFSSAHLKGDLATKVSTLREYERARKEQIAQLGLPMGERDFLITVRMTRGWDDLREKWRRSGDKGQEALHAIIATEQTRKRGRFGDPDLFRWLARPENHHVWADGHADAVGVLARVNAMERLVERSRDTALMTLPDPVAHPRSAQWEAEGGSNLRNYQLEAVGGELQITLPLLKAADDGRCIDTPL

SEQ ID NO: 194 - protein, 1501 aa, unidentified
MPQQAKPPVTQRAYTLRLRGADSNDPSWRDALWQTHEAVNRGAQAFGDWLLTLRGGLDHTLADTPVKGGKGKPDPDPTDEERKARRILLALSWLSVESKLGAPAGLIIAFGTEAAEERNRKVVAALEEILKSRGVDQNEINAWKKDCSASLSAAIRDDAVWVNRSKAFDEAVESIGSSGSSGSSLTREEPWDMLERFFGSRDAYLAPAKGSEDESSEAKQEDQAKDLVQKAGQWLSSRFGTGKGADFRRMATVYEAIAKWDGKASLEMAGDKAIADLATALSEFNPASNDLQGVLGLISGPGYKSATRNFLNQLAAQTTVTQQDFVSLKDKANNDAQECKQNTGSKGQRPYSNSILEKVESVCGFTYLQDGGPARHSEFAVILDHAARRVSLAHTWIKLAEAERRKFEEDAKKIDQVPEAAKDWLDRFCLERSGVSGALEPYRIRRRAVDGWKEVVAEWSKSDCKTVEDRIAAARALQDDPEIDKFGDIQLFEALAEDDAVCVWHKDGDAAKAPDPQPLIDYALAAEAEFKKRHFKVPAYRHPDALLHPIFCDFGKSRWDICFDVHKNMQTPFPRALCLTLWTGSEMKRIPLCWQSKRLARDLALGNNTGDAGASEVTRADRLGRAASRAASNVTKSDVVNIAGLFEQADWNGRLQAPRQQLEAIARYVEKHDWDQKAEKMRNAIQWLVTFSARLQPQGPWCAYAKIHGLKEDPQYWPHADTNKNRKGHARLILSRLPGLRVLAVDLGHRYAAACAVWEALSTEAFQREIKGRTILRGRTDGNALYCHTRHKANGKERVTIYRRIGADTLPDGKPHPAPWARLDRQFLIKLQGEEEGVREASNEEIWAVHQLEAALGRPVSLIDRLVASGWGGSDKQKARLEGLKQLGWDPADKPSLSVDELMSSAVRTMRLALKRHGDRARIAHYLITDEKTTPGGIKETLDEKGRIDLLQDALVLWHDLFSSRGWRDDTAKQLWNAHVAKLHGYKAPEEPGEDSSGAERKKKQRENREKLYDVAKALAQDVTLREALHDAWKKRWENDDERWKKQLRWFKDWVFPRGNHASDPTIRKRQLINPSGGNGRRGNHASDPTIRKRQLINPSGGNGRRGNHASDPTIRKVGGLSLPRLATLTEFRRKVQVGFFTRLKPDGTRAETKEQFGQSALDALEHLREQRVKQLASRIAEAALGVGRVRRPVEGKDPKRPDVRVDEPCHAIVIEDLTHYRPEETRTRRENRQLMTWSSSKVKKYLAEACQLHGLHLREVSASYTSRQDSRTGAPGVRCQDVPVKEFMRSPFWRKQVKQAEAKQAANKGDARERLLCDLNARWKDRTAADWEKAGAVRIPLQGGEIFVSADANSPAAKGIQADLNAAANIGLRALTDPDWAGKWWYVPCDPASFRPVRDKVDGSAVVNPDQPLRQSAQAQSGDAAKDKNGNKGAGKSKEVVNLWRDISSSPLECIEFGEWKEYAAYQNEVQCRVIRILKEQIKGRDKQPHEGSKEDDIPL

SEQ ID NO: 195 - protein, 1149 aa, Desulfovibrio inopinatus
MPTRTINLKLVLGKNPENATLRRALFSTHRLVNQATKRIEEFLLLCRGEAYRTVDNEGKEAEIPRHAVQEEALAFAKAAQRHNGCISTYEDQEILDVLRQLYERLVPSVNENNEAGDAQAANAWVSPLMSAESEGGLSVYDKVLDPPPVWMKLKEEKAPGWEAASQIWIQSDEGQSLLNKPGSPPRWIRKLRSGQPWQDDFVSDQKKKQDELTKGNAPLIKQLKEMGLLPLVNPFFRHLLDPEGKGVSPWDRLAVRAAVAHFISWESWNHRTRAEYNSLKLRRDEFEAASDEFKDDFTLLRQYEAKRHSTLKSIALADDSNPYRIGVRSLRAWNRVREEWIDKGATEEQRVTILSKLQTQLRGKFGDPDLFNWLAQDRHVHLWSPRDSVTPLVRINAVDKVLRRRKPYALMTFAHPRFHPRWILYEAPGGSNLRQYALDCTENALHITLPLLVDDAHGTWIEKKIRVPLAPSGQIQDLTLEKLEKKKNRLYYRSGFQQFAGLAGGAEVLFHRPYMEHDERSEESLLERPGAVWFKLTLDVATQAPPNWLDGKGRVRTPPEVHHFKTALSNKSKHTRTLQPGLRVLSVDLGMRTFASCSVFELIEGKPETGRAFPVADERSMDSPNKLWAKHERSFKLTLPGETPSRKEEEERSIARAEIYALKRDIQRLKSLLRLGEEDNDNRRDALLEQFFKGWGEEDVVPGQAFPRSLFQGLGAAPFRSTPELWRQHCQTYYDKAEACLAKHISDWRKRTRPRPTSREMWYKTRSYHGGKSIWMLEYLDAVRKLLLSWSLRGRTYGAINRQDTARFGSLASRLLHHINSLKEDRIKTGADSIVQAARGYIPLPHGKGWEQRYEPCQLILFEDLARYRFRVDRPRRENSQLMQWNHRAIVAETTMQAELYGQIVENTAAGFSSRFHAATGAPGVRCRFLLERDFDNDLPKPYLLRELSWMLGNTKVESEEEKLRLLSEKIRPGSLVPWDGGEQFATLHPKRQTLCVIHADMNAAQNLQRRFFGRCGEAFRLVCQPHGDDVLRLASTPGARLLGALQQLENGQGAFELVRDMGSTSQMNRFVMKSLGKKKIKPLQDNNGDDELEDVLSVLPEEDDTGRITVFRDSSGIFFPCNVWIPAKQFWPAVRAMIWKVMASHSLG

SEQ ID NO: 196 - protein, 1132 aa, Tuberibacillus calidus
MATKSFILKMKTKNNPQLRLSLWKTHELFNFGVAYYMDLLSLFRQKDLYMHNDEDPDHPVVLKKEEIQERLWMKVRETQQKNGFHGEVSKDEVLETLRALYEELVPSAVGKSGEANQISNKYLYPLTDPASQSGKGTANSGRKPRWKKLKEAGDPSWKDAYEKWEKERQEDPKLKILAALQSFGLIPLFRPFTENDHKAVISVKWMPKSKNQSVRKFDKDMFNQAIERFLSWESWNEKVAEDYEKTVSIYESLQKELKGISTKAFEIMERVEKAYEAHLREITFSNSTYRIGNRAIRGWTEIVKKWMKLDPSAPQGNYLDVVKDYQRRHPRESGDFKLFELLSRPENQAAWREYPEFLPLYVKYRHAEQRMKTAKKQATFTLCDPIRHPLWVRYEERSGTNLNKYRLIMNEKEKVVQFDRLICLNADGHYEEQEDVTVPLAPSQQFDDQIKFSSEDTGKGKHNFSYYHKGINYELKGTLGGARIQFDREHLLRRQGVKAGNVGRIFLNVTLNIEPMQPFSRSGNLQTSVGKALKVYVDGYPKVVNFKPKELTEHIKESEKNTLTLGVESLPTGLRVMSVDLGQRQAAAISIFEVVSEKPDDNKLFYPVKDTDLFAVHRTSFNIKLPGEKRTERRMLEQQKRDQAIRDLSRKLKFLKNVLNMQKLEKTDEREKRVNRWIKDREREEENPVYVQEFEMISKVLYSPHSVWVDQLKSIHRKLEEQLGKEISKWRQSISQGRQGVYGISLKNIEDIEKTRRLLFRWSMRPENPGEVKQLQPGERFAIDQQNHLNHLKDDRIKKLANQIVMTALGYRYDGKRKKWIAKHPACQLVLFEDLSRYAFYDERSRLENRNLMRWSRREIPKQVAQIGGLYGLLVGEVGAQYSSRFHAKSGAPGIRCRVVKEHELYITEGGQKVRNQKFLDSLVENNIIEPDDARRLEPGDLIRDQGGDKFATLDERGELVITHADINAAQNLQKRFWTRTHGLYRIRCESREIKDAVVLVPSDKDQKEKMENLFGIGYLQPFKQENDVYKWVKGEKIKGKKTSSQSDDKELVSEILQEASVMADELKGNRKTLFRDPSGYVFPKDRWYTGGRYFGTLEHLLKRKLAERRLFDGGSSRRGLFNGTDSNTNVE

SEQ ID NO: 197 - protein, 1108 aa, Bacillus thermoamylovorans
MATRSFILKIEPNEEVKKGLWKTHEVLNHGIAYYMNILKLIRQEAIYEHHEQDPKNPKKVSKAEIQAELWDFVLKMQKCNSFTHEVDKDVVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPFTDSNEPIVKEIKWMEKSRNQSVRRLDKDMFIQALERFLSWESWNLKVKEEYEKVEKEHKTLEERIKEDIQAFKSLEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKFVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKLVTTHADINAAQNLQKRFWTRTHGFYKVYCKAYQVDGQTVYIPESKDQKQKIIEEFGEGYFILKDGVYEWGNAGKLKIKKGSSKQSSSELVDSDILKDSFDLASELKGEKLMLYRDPSGNVFPSDKWMAAGVFFGKLERILISKLTNQYSISTIEDDSSKQSM

SEQ ID NO: 198 - protein, 1108 aa, Bacillus sp.
MAIRSIKLKLKTHTGPEAQNLRKGIWRTHRLLNEGVAYYMKMLLLFRQESTGERPKEELQEELICHIREQQQRNQADKNTQALPLDKALEALRQLYELLVPSSVGQSGDAQIISRKFLSPLVDPNSEGGKGTSKAGAKPTWQKKKEANDPTWEQDYEKWKKRREEDPTASVITTLEEYGIRPIFPLYTNTVTDIAWLPLQSNQFVRTWDRDMLQQAIERLLSWESWNKRVQEEYAKLKEKMAQLNEQLEGGQEWISLLEQYEENRERELRENMTAANDKYRITKRQMKGWNELYELWSTFPASASHEQYKEALKRVQQRLRGRFGDAHFFQYLMEEKNRLIWKGNPQRIHYFVARNELTKRLEEAKQSATMTLPNARKHPLWVRFDARGGNLQDYYLTAEADKPRSRRFVTFSQLIWPSESGWMEKKDVEVELALSRQFYQQVKLLKNDKGKQKIEFKDKGSGSTFNGHLGGAKLQLERGDLEKEEKNFEDGEIGSVYLNVVIDFEPLQEVKNGRVQAPYGQVLQLIRRPNEFPKVTTYKSEQLVEWIKASPQHSAGVESLASGFRVMSIDLGLRAAAATSIFSVEESSDKNAADFSYWIEGTPLVAVHQRSYMLRLPGEQVEKQVMEKRDERFQLHQRVKFQIRVLAQIMRMANKQYGDRWDELDSLKQAVEQKKSPLDQTDRTFWEGIVCDLTKVLPRNEADWEQAVVQIHRKAEEYVGKAVQAWRKRFAADERKGIAGLSMWNIEELEGLRKLLISWSRRTRNPQEVNRFERGHTSHQRLLTHIQNVKEDRLKQLSHAIVMTALGYVYDERKQEWCAEYPACQVILFENLSQYRSNLDRSTKENSTLMKWAHRSIPKYVHMQAEPYGIQIGDVRAEYSSRFYAKTGTPGIRCKKVRGQDLQGRRFENLQKRLVNEQFLTEEQVKQLRPGDIVPDDSGELFMTLTDGSGSKEVVFLQADINAAHNLQKRFWQRYNELFKVSCRVIVRDEEEYLVPKTKSVQAKLGKGLFVKKSDTAWKDVYVWDSQAKLKGKTTFTEESESPEQLEDFQEIIEEAEEAKGTYRTLFRDPSGVFFPESVWYPQKDFWGEVKRKLYGKLRERFLTKAR

SEQ ID NO: 199 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 200 - protein, 1115 aa, Alicyclobacillus hesperidum
MTVRSIRVKLAVGSPQYRDVRRGLWKTHEIMNQGVRYYCEWLVLMRQEPIYDEDEHGLTVVQRTREDIQAELLSRLRTLQSAHQHSGDMGTDEELLSLMRQLYEQLVPSSVDKNKSGDARMIARNFFNPLTNPNSQGGLGISNAGRKPKWLLKKLSGDPTWEEDYKKAMEQKQESSVSFLLLELRRFGLHPIFLPYTDTVLEVSWAPKKARQWVRKWDYDLFQQSIERMLSWESWTRRVKERFEKLVESEKKFYDENFATDPEFIKLAETLEGELQASSQGFVAVDEHAFQIRPRSMRGFDRVADEWCKLADDAPIEEYEAAIKRVQARLGRNFGSYVLFAHLAKPEYWSLWRSDPTKILRFARLRALQRAVARAKRHARLTLPDAIHHPIWIRYDAKGKNIYSYRLLIPEKRSKRYYVEFSSLIMPDGENRWAEHRNIRVPLAFSRQWERLHFSIMEDGSLCVQYRDPGVDEPLRAELGGAKIQFDRRYLIRRSSTLSAGECGPVYLNVSVDVNPAHRPDVQVLQSAKLVSVSRDTNRIYLRPENLSAYWKSQGDGTLPLRVMSVDLGVRSSAAVVICRLEHRDSVVSSGRRTATIYRIAGTDEFVAVQERAFLLRLPGEGKGTNEDAPLRDVYAQLGTIRQGIQILRSLLRLCDTKTPDERQEALHGLAQSLEPSGAWKDELHPHLVMLQGVVHDSVDNWKQKVISVHRQMERILGHAVREWKVARKNAGKPPIRRGAGGLSLRRIRQLEQERRTLVAWSNHAREPGQVVRIKRGTQVAQWLVERVNHLKEDRLKKLADLLIMTALGYVYDETKPSGHKWDKRYPPCQIILMEDLSRYRFQSDRPPSENSQLMAWSHRRLLEILKLQADLHKLIVGTVFPAFSSRFDAQSGAPGVRCRSVKKQDIENAAQGKGWLARELQRLNWTLEWLQPNDLIPTGDGELFVTPACCDRQKGIKIVHADLNAAQNLQRRFWGGHAESLCRVTCDVVERDGRRYAVPRISNAFADSFYKVFGQGVFVSTDEEDVYRWMVGEKISSRGRSRGRTSDEEAEAETWIDEAREQQGKVIALFRDASGQIHGGDWLVAKVFWGWVERLVTARLLSRMSEREAAAHKE

SEQ ID NO: 201 - protein, 1129 aa, Alicyclobacillus acidiphilus
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 202 - protein, 1144 aa, Alicyclobacillus macrosporangiidus
MAVKSIKVKLMLGHLPEIREGLWHLHEAVNLGVRYYTEWLALLRQGNLYRRGKDGAQECYMTAEQCRQELLVRLRDRQKRNGHTGDPGTDEELLGVARRLYELLVPQSVGKKGQAQMLASGFLSPLADPKSEGGKGTSKSGRKPAWMGMKEAGDSRWVEAKARYEANKAKDPTKQVIASLEMYGLRPLFDVFTETYKTIRWMPLGKHQGVRAWDRDMFQQSLERLMSWESWNERVGAEFARLVDRRDRFREKHFTGQEHLVALAQRLEQEMKEASPGFESKSSQAHRITKRALRGADGIIDDWLKLSEGEPVDRFDEILRKRQAQNPRRFGSHDLFLKLAEPVFQPLWREDPSFLSRWASYNEVLNKLEDAKQFATFTLPSPCSNPVWARFENAEGTNIFKYDFLFDHFGKGRHGVRFQRMIVMRDGVPTEVEGIVVPIAPSRQLDALAPNDAASPIDVFVGDPAAPGAFRGQFGGAKIQYRRSALVRKGRREEKAYLCGFRLPSQRRTGTPADDAGEVFLNLSLRVESQSEQAGRRNPPYAAVFHISDQTRRVIVRYGEIERYLAEHPDTGIPGSRGLTSGLRVMSVDLGLRTSAAISVFRVAHRDELTPDAHGRQPFFFPIHGMDHLVALHERSHLIRLPGETESKKVRSIREQRLDRLNRLRSQMASLRLLVRTGVLDEQKRDRNWERLQSSMERGGERMPSDWWDLFQAQVRYLAQHRDASGEAWGRMVQAAVRTLWRQLAKQVRDWRKEVRRNADKVKIRGIARDVPGGHSLAQLDYLERQYRFLRSWSAFSVQAGQVVRAERDSRFAVALREHIDNGKKDRLKKLADRILMEALGYVYVTDGRRAGQWQAVYPPCQLVLLEELSEYRFSNDRPPSENSQLMVWSHRGVLEELIHQAQVHDVLVGTIPAAFSSRFDARTGAPGIRCRRVPSIPLKDAPSIPIWLSHYLKQTERDAAALRPGELIPTGDGEFLVTPAGRGASGVRVVHADINAAHNLQRRLWENFDLSDIRVRCDRREGKDGTVVLIPRLTNQRVKERYSGVIFTSEDGVSFTVGDAKTRRRSSASQGEGDDLSDEEQELLAEADDARERSVVLFRDPSGFVNGGRWTAQRAFWGMVHNRIETLLAERFSVSGAAEKVRG

SEQ ID NO: 203 - protein, 1027 aa, Sulfobacillus thermosulfidooxidans
RQSREDASPQIIISASDLKADLLYHARQQQKEHVPRITGSDAEVLGALRQVYELIVPSSVGKSGDSKTIARKFLSPLTDPDSAGGRDQSASGRKPTWTKMKAEGNPLWEEKFRQWKDRKDNDPTPFVLNQLADYGLLPLIRLFTDVGENIFDPKKPGQFVRPWDRSMFQQAIERLMSWESWNQRVRQEWEALTQKHSAFYREQFTAEPDAALYRVAQSLEEEMRKEHQGFATDAPEAFRIRRVALKGFDRLLERWQKTLGKNGQSATLLDDIRRVQSDLGDKFGSAPLYQKLVDERWQRLWTVDPTFLQRYAAFNDLTQRLQRAKRVANLTLPDAVAHPIWSRYEGPNASSGNRYHIHLPTTGQPSSVTFDRILWPDGDGGWYERKRVTVFLRPSHQVDRIREAPTDSVVDNFPLVVEDQSARTILRASWGGAKLEYDRNRLPRQLKKGVPDSIYLSLTLNLDTTKPSGLFHMQQNGRVWIRKDVVMQYYNEIPGDNVQFKPLYVMSVDLGIRSAAAVSIFSVQLKTGIEEHRLTYPVADCPGLVAVHERSVLLTMPGERREQRDRRYEQQRQGLRELRTDMRGMNDLLRGAYVDGDRREEFLARLSKLEETSPELWEPVYRSLNDSKMAPAAEWERLVVYCHRQVEQSLSSRIQNLRSGRSAYRMSGGLSLDHVQDLERIRGIIASWTNHPRIPGSVVRWQQGRSHTVALGRHILELKRDRVKKVANYLIMTALGYAYDSKRARGEKWVRRYPSCHLMVFEDLTRYRFRTDRPRSENRQLMRWTHQELIAVTGIQAEPHGILVGTMYAGFSSRFDAVTKAPGVRGATVRQILRTRGMVRLKEIAADVGVDINTLRPHDVLPTGDGEYLLSVVRHRDSYRLKQVHADINAAHNLQRRLWTQDEVFRVSCRLALNSERVVATPPPSYNKRYGKGFFEKGDNGVYIWKTGGKIKISDMLEEDMDIPEDTAELLRGNSVTLFRDPSGTIAGGNWLEAKEFWGRVNSLVNKGVRDKILGGIPVDNSSAHAE

SEQ ID NO: 204 - protein, 1070 aa, Spirochaeta sp.
MGLLLPSLSRTVNVTIHLILHPRKKGSRHREYAVMLDHAVRKIFLAHNWIKRAEAERQKFEADLYKIDRVPQEARDWLDEFCRERTESTGSIDGYHIRRKAVLGWEALVEAWDQKDCLSVEDRIAAARDLQDNPGMDKFGDIWLYEALASAPCVWQKDGEPNAQILLDYVDAGEAEYKRSHYKVPAYRHPDPLLHPIFCDFGQSRWSISFDIHEFKKNGEKNPVNIHALTMGLVSKKRIVKTELKWSSKRLNSNLALSLESPEDAIEVSRATRLGRAAVGASQDRAVNIAGLFESAGWNGRLQAPRKQLEALAKLEEDKSAEALAKALRNRIKWFITFSPKLQPHGPWMEYAERFSGEAPSRAAVIKGKYTVIHQDKTRRRPLAKLHLCRMPGLRVLSVDLGHRHAAACAVWETLSSESMEKKCREAGCLPPAPEDLYLHLKKKNKTAVYRRIGGNFLPDGNEHPAPWAKLDRQFIIDLQGEEGCTRMALAGEIWQVHCMEKVFGRSIPLVDRLVRAGWGEKNKQPEILQELKQKGWVPLEVSKTNTGYHYSLCVDSLMTLAVNTVRFALRRHACRARIAYYMEGGAIPEGGLPENSGNKDFIVEALMLWYELATDSRWNGSWEANFWDENFDKKLAEIQDAVNEREGDKAKIIKQKERKELLKKEFIPLAEGLLENSRRISIASQWRMVWNEEDAIWQSELRSLRDWILPKGTRGKKRTIRHVGGLSLSRLAVIKSLYRVQKSFYTRMKPEGEPMDGTMAVGEGFGQKILDDLETMKEQRVKQLASRVVEAALGTGRIKKPENNKTPKRPFTAVDEPCHAVVIENLTHYRPENKRTRRENRQLMTWSSSKVKKYLFESCQLHGLYLFEVQASYTSRQDSRTGAPGVRCSELSVKKFLESPFRQREIAHAEENMAQENPCNRYLIALHNKWKNREYDKTAPPLRIPHWGGEIFVSALTGNTLQADLNAAANIGLQALLDPDWPGRWWYVPAVKGCDGRRIPHSKCSGAACLDNWRVGLKNNLYTGVRTPLPGKNKGSTSGEDVHKSNAVEKSTINLWRDISVLPLTEGQW

SEQ ID NO: 205 - protein, 1108 aa, synthetic construct
MATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYGERSRFENSRLMKWSRREIPRQVALQGEIYGLQVGEVGAQFSSRFHAKTGSPGIRCRVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSM

SEQ ID NO: 206 - protein, 1252 aa, unidentified
MTKLRHRQKKLTHDWAGSKKREVLGSNGKLQNPLLMPVKKGQVTEFRKAFSAYARATKGEMTDGRKNMFTHSFEPFKTKPSLHQCELADKAYQSLHSYLPGSLAHFLLSAHALGFRIFSKSGEATAFQASSKIEAYESKLASELACVDLSIQNLTISTLFNALTTSVRGKGEETSADPLIARFYTLLTGKPLSRDTQGPERDLAEVISRKIASSFGTWKEMTANPLQSLQFFEEELHALDANVSLSPAFDVLIKMNDLQGDLKNRTIVFDPDAPVFEYNAEDPADIIIKLTARYAKEAVIKNQNVGNYVKNAITTTNANGLGWLLNKGLSLLPVSTDDELLEFIGVERSHPSCHALIELIAQLEAPELFEKNVFSDTRSEVQGMIDSAVSNHIARLSSSRNSLSMDSEELERLIKSFQIHTPHCSLFIGAQSLSQQLESLPEALQSGVNSADILLGSTQYMLTNSLVEESIATYQRTLNRINYLSGVAGQINGAIKRKAIDGEKIHLPAAWSELISLPFIGQPVIDVESDLAHLKNQYQTLSNEFDTLISALQKNFDLNFNKALLNRTQHFEAMCRSTKKNALSKPEIVSYRDLLARLTSCLYRGSLVLRRAGIEVLKKHKIFESNSELREHVHERKHFVFVSPLDRKAKKLLRLTDSRPDLLHVIDEILQHDNLENKDRESLWLVRSGYLLAGLPDQLSSSFINLPIITQKGDRRLIDLIQYDQINRDAFVMLVTSAFKSNLSGLQYRANKQSFVVTRTLSPYLGSKLVYVPKDKDWLVPSQMFEGRFADILQSDYMVWKDAGRLCVIDTAKHLSNIKKSVFSSEEVLAFLRELPHRTFIQTEVRGLGVNVDGIAFNNGDIPSLKTFSNCVQVKVSRTNTSLVQTLNRWFEGGKVSPPSIQFERAYYKKDDQIHEDAAKRKIRFQMPATELVHASDDAGWTPSYLLGIDPGEYGMGLSLVSINNGEVLDSGFIHINSLINFASKKSNHQTKVVPRQQYKSPYANYLEQSKDSAAGDIAHILDRLIYKLNALPVFEALSGNSQSAADQVWTKVLSFYTWGDNDAQNSIRKQHWFGASHWDIKGMLRQPPTEKKPKPYIAFPGSQVSSYGNSQRCSCCGRNPIEQLREMAKDTSIKELKIRNSEIQLFDGTIKLFNPDPSTVIERRRHNLGPSRIPVADRTFKNISPSSLEFKELITIVSRSIRHSPEFIAKKRGIGSEYFCAYSDCNSSLNSEANAAANVAQKFQKQLFFEL

SEQ ID NO: 207 - protein, 1050 aa, unidentified
MRSNYHGGRNARQWRKQISGLARRTKETVFTYKFPLETDAAEIDFDKAVQTYGIAEGVGHGSLIGLVCAFHLSGFRLFSKAGEAMAFRNRSRYPTDAFAEKLSAIMGIQLPTLSPEGLDLIFQSPPRSRDGIAPVWSENEVRNRLYTNWTGRGPANKPDEHLLEIAGEIAKQVFPKFGGWDDLASDPDKALAAADKYFQSQGDFPSIASLPAAIMLSPANSTVDFEGDYIAIDPAAETLLHQAVSRCAARLGRERPDLDQNKGPFVSSLQDALVSSQNNGLSWLFGVGFQHWKEKSPKELIDEYKVPADQHGAVTQVKSFVDAIPLNPLFDTTHYGEFRASVAGKVRSWVANYWKRLLDLKSLLATTEFTLPESISDPKAVSLFSGLLVDPQGLKKVADSLPARLVSAEEAIDRLMGVGIPTAADIAQVERVADEIGAFIGQVQQFNNQVKQKLENLQDADDEEFLKGLKIELPSGDKEPPAINRISGGAPDAAAEISELEEKLQRLLDARSEHFQTISEWAEENAVTLDPIAAMVELERLRLAERGATGDPEEYALRLLLQRIGRLANRVSPVSAGSIRELLKPVFMEEREFNLFFHNRLGSLYRSPYSTSRHQPFSIDVGKAKAIDWIAGLDQISSDIEKALSGAGEALGDQLRDWINLAGFAISQRLRGLPDTVPNALAQVRCPDDVRIPPLLAMLLEEDDIARDVCLKAFNLYVSAINGCLFGALREGFIVRTRFQRIGTDQIHYVPKDKAWEYPDRLNTAKGPINAAVSSDWIEKDGAVIKPVETVRNLSSTGFAGAGVSEYLVQAPHDWYTPLDLRDVAHLVTGLPVEKNITKLKRLTNRTAFRMVGASSFKTHLDSVLLSDKIKLGDFTIIIDQHYRQSVTYGGKVKISYEPERLQVEAAVPVVDTRDRTVPEPDTLFDHIVAIDLGERSVGFAVFDIKSCLRTGEVKPIHDNNGNPVVGTVAVPSIRRLMKAVRSHRRRRQPNQKVNQTYSTALQNYRENVIGDVCNRIDTLMERYNAFPVLEFQIKNFQAGAKQLEIVYGS

SEQ ID NO: 208 - protein, 1273 aa, unidentified
MKKFELKQNFRNNYSGKTLRNFRQTLAQIANKKSSDSILTIKFKLDCSKTGKLPKYENLISLYDTIEDIKKGTLSYYLFTLIVSGFKFFGSASQAKAFSTKDIFKDNDFYNQFKIQSHLDLPDFVPSKIYQRLKKNVRSTNGKDNAFKASVIVAEYRKEIGKLKNKDESSEHQCEELFKKIGTALETRFSSWQDLINNCSTGCEIIDEILNDSFGTLPSIKKMVLASTTQSSDGEQDGIAIAYDPDSTFIKSDELLNPYFAVATILKSMPPEIQQDKKSAYVKANLTTPTHNALSWIFGKGLTLFQTESTEKLCAMFNVSDKRVIEQVQDAAKAVKLPAELDLNHCTLKFQDFRSSLGGHLDSWTTNYLKRLDELNDLLLNLPKNLSLPDIFMIDGKDFIEYSGCNRDEIQQMIDFVVNEQNRIKLQESLNALLGKGNNQICSDDISTVKDFSEIVNSLHSFVQQIDNSLEQSSNEANSIFSELKKKIEKNEKWDIWKNNLKKIPKLNKLSGGVPDAWKEIREIEQKFHEISENQKKHFTEVMEWIDAGNGTIDIFESRFKYDELLKKSKKNNLQSADELAFRSVLNKLGRFARQGNDLVCEKIKNWFKEQNIFDSSKDFNRYFINQKGFIFKHPSSKKDNSPYNLSANLLEKRYEVTNTVGALLEQCESDPAIVNDPFSMRSLVEFRALWFSINISGISKEQHIPTKIAQPKLDDSTYQESVSPTLKYRLEKEQITSSELNSIFTVYKSLLSGLSIRLSRNSFYLRTKFSWIGNNSLIYCPKETTWKIPAAYFKSDLWNEYKDKQILIVNEEYDVDVVKTFESVYKIVKSKDNNEKNRILPLLKQLPHDWMFKLPFGASNAEKCKVLKLEKNNKKFKPLSVSKDSLARLSGPSTYFNQIDEIMMNDESELSEMTLLADEPVRQQMSNGKIEIIPDDYVMSLAIPITRSLKKGNTESFPFKNIVSIDQGEAGFAYAVFKLSDCGNERAEPIATGLIPIPSIRRLIHSVKKYRGKKQRIQNFNQKFDSTMFTLRENVTGDICGLIVALMKKYNAFPILEKQVGNLESGSKQLMLVYKAVNSKFLAAKVDMQNDQRRSWWYQGNSWNTPILRISNPNQSNNKNIVKNINGKKYEELKIYPGYSVSAYMTSCICHVCGRNALELLKNDDSTGKVKKYQINQDGEVTIGGEVIKLYRKPDRLTPVKNLAKKGNRERTYASINERAPMSKDTTQSRYFCVFKNCPCHNKEQHADVNAAINIGRRFLKDCILDDNKEKD

SEQ ID NO: 209 - protein, 1258 aa, unidentified
MNARDWRKHVGVLAQQHKETTRTYTFPLDTTGSAIDFDAALQAYNAVEGVGYGSLLGLACAVHLSGFRLFSTGKEAATFRNRARYPNAAFQAALRKELGTTITTLTPETLDRLFSSRPKRRNGVPLPWNQDSIRDRLYTNWVKPRPGDTPDAVLFQIATGIAQEITEDVSSWTDLAKNSDRGLKAAHRYFARVGGFPAFDNLTPPATVQPTDTTIDYDPNAPFHLVSHADQTLIHQSISLCAHRIRQEDPALDPNKSGFIKQLQNNFLSQTFYGLSWLFGAGYVHFRECTANDLAIQYGIPNNCRDGIHQIKSFADAILPNTFFEKKHYRKDSRSVGKKAKSWISNYWQRLLQLQTWVDDHTWVTLPQELTEAQFKPLFRGLLVDAVELMAIAERLPQRLADCRDSLDCLMGKGPQAATKNDVEIVEKVREEIESFVGQIEQLGNQLRHQLENENNDQVHRDNLHQLKNRLPLDLRRPQALNKISGGVPDVAKSIRGLETQLDQVLKERRSHFGRLTKWAKECGITLDPLQPLIESEKQRVAERGSAHDAKELAIRLLLQRIGRLGHRLSPTNATAIQELLRPVFAVKREFNLFFHNHMGALYRSPYSTSRHQPFQINVDVAHGTDWIGTIETLIQNLFTQIQDDALLRDLVQLEGFVFSHKLRALPGVIPSELARPNNLQQMGLPALLLVLLQADQVHRETVLRVFNLYGSAINGYLFQALRPGFIVRAGFQRLETKKLRYVPKAQSWQYPDRLHHAKSAIKNSLSAGWIKKNHQGAILPQKTLTALVKQKSLKDTGVPEYLVQAPHDWYVPIDLRGPAIPIEGLTVGTEGPELTQLGPMKDDCAFRAIGPSSFKSKIDAGLLPQDVKYGDMTLIFDQHYQQSISFANGTFSIQYQPTSLQVKAAIPVVDKRPRDTRNNSHLYDRIVAIDLGERKIGYAIFDLKQVLKSEQLEPMREDGKPLIGSISIRSIRGLMKAVQTHRNRRQPNYRIDQTYSKALMHYRESVIGDVCNAIDTLCARYGGFPVLESSVRNFEVGSAQLKTVYGSVSRRYTWSAVDAHKNQRQQYWLGGTKDKIPIWTHPYLMTREWDEKNSKWSNRSKPLKMHPGVEVHPAGTSQICHQCKRNPIGALWNVADTVVLDDQGQLDLDDGTIRLNSGYIDTTEIKRARRKKIRLPENKPLTGSHKTSHVRAVARRNLRQPPKSTRAKDTTQSRYTCLYVDCGHECHADENAAINIGRKYLQERIHIEASRQALSTR

SEQ ID NO: 210 - protein, 1269 aa, unidentified
MVAGLKKIKRDGVTMKSNYHGGVKARAWRKRIGGLARRQKETVFTYKFPLETEEAGIDFDKAVQTYGIAEGISQGSLIGLVCAFHLSGFRLFSKADETKAFCNQGRYPNQAFAEKLRNELSVTLPKLSPQSLDVLFQSSPKSKNGVAPEWSKNAIRNRLYTNWTGKGAGTNPDEHLLEIAEDIAAEIDSDLDGWKDLEEHPEKGLSAADRYFQAQGDFPSLTGLPPSVPLTPQNSTVAFEGDPVCLNPSDNTLLHQAVARCAGRILQEQPNLSPDKNRFINQLQDELVSSQNNGLSWLFGVGFKYWKEMSVDQLADDYKVKSTDLDALKQVKSFIDAIPLNPLFDTPHYGEFRASVAGKMRSWVKNYWKRLLDLKSQLGTANINLPEGLDEQRAENLFSGLLIDSKGLRQVTDKLPSRLKKAEDTIDRLMGDGNPTSDDIEQVETVAAEISAFIGQVEQFNNQLEQRLENPLEGDDETFLKQLKIDLPAEFKKPPAINRISGGSPDPTAEIAELEEKLDRLMSARKEHYETIAEWASANKVTLDPMEAMTTLEAQRLTERGAEGDQEEFALRLLLQRIGRLANRLSPQGATAIRDLLRPVFTEKREFNLFFHNRMGSLYRSPYSTSRHQPFTIDVAVAKNTDWMDALDGIAETIMKGLSQAGDELSLRQLEEDEVSREVCLKAFNLYVSAINGCLFRALREGFIVRTKFQRLERDVLSYVPKTKLWNYPQRLDTARGPIHSALAAAWINKEGSVIDPVETVTALSDTGFSDDGIPEYLVQAPHDWYLRDWINISGFSLSQRLRGLPDTVPGELALVRSADDVRIPPMLALTPIDLRDISKPVSGLPVKKNITGLKRQKKQTAFRMVGPSSFKSHLDSTLLSEEVKLGDFTLIFDQYYKQRVSYNGRVKITFEPDRLHVEAAVPVIDKRVRPSTEEDALFDHLLAIDLGEKRVGYAVYDIKACLRTGDIKPLEDGDGKPIVGSVAVPSIRRLMKAVRSHRQQRQPNQKVNQTYSTALMNYRENVIGDVCNRIDTLMEKYNAFPVLESSVMNFEAGSRQLEMVYGSVLHRYTYSKIDAHTAKRKEYWYTGEYWDHPYLMAHKWNERTRSYSGSLSALTLYPGVMVHPAGTSQRCHQCKRNPMVEIKQLTGQVEINADGSLELDDGTICLYEGYDYSPEEYKKAKREKRRLDPNVPLSGRHQAKHVSAVAKRNLRRPTVSMMSGDTTQARYVCLYTDCDFTGHADENAAINIGWKYLTERIALSESKDKAGV

SEQ ID NO: 211 - protein, 1210 aa, unidentified
MSKRHPRISGVKGYRLHAQRLEYTGKSGAMRTIKYPLYSSPSGGRTVPREIVSAINDDYVGLYGLSNFDDLYNAEKRNEEKVYSVLDFWYDCVQYGAVFSYTAPGLLKNVAEVRGGSYELTKTLKGSHLYDELQIDKVIKFLNKKEISRANGSLDKLKKDIIDCFKAEYRERHKDQCNKLADDIKNAKKDAGASLGERQKKLFRDFFGISEQSENDKPSFTNPLNLTCCLLPFDTVNNNRNRGEVLFNKLKEYAQKLDKNEGSLEMWEYIGIGNSGTAFSNFLGEGFLGRLRENKITELKKAMMDITDAWRGQEQEEELEKRLRILAALTIKLREPKFDNHWGGYRSDINGKLSSWLQNYINQTVKIKEDLKGHKKDLKKAKEMINRFGESDTKEEAVVSSLLESIEKIVPDDSADDEKPDIPAIAIYRRFLSDGRLTLNRFVQREDVQEALIKERLEAEKKKKPKKRKKKSDAEDEKETIDFKELFPHLAKPLKLVPNFYGDSKRELYKKYKNAAIYTDALWKAVEKIYKSAFSSSLKNSFFDTDFDKDFFIKRLQKIFSVYRRFNTDKWKPIVKNSFAPYCDIVSLAENEVLYKPKQSRSRKSAAIDKNRVRLPSTENIAKAGIALARELSVAGFDWKDLLKKEEHEEYIDLIELHKTALALLLAVTETQLDISALDFVENGTVKDFMKTRDGNLVLEGRFLEMFSQSIVFSELRGLAGLMSRKEFITRSAIQTMNGKQAELLYIPHEFQSAKITTPKEMSRAFLDLAPAEFATSLEPESLSEKSLLKLKQMRYYPHYFGYELTRTGQGIDGGVAENALRLEKSPVKKREIKCKQYKTLGRGQNKIVLYVRSSYYQTQFLEWFLHRPKNVQTDVAVSGSFLIDEKKVKTRWNYDALTVALEPVSGSERVFVSQPFTIFPEKSAEEEGQRYLGIDIGEYGIAYTALEITGDSAKILDQNFISDPQLKTLREEVKGLKLDQRRGTFAMPSTKIARIRESLVHSLRNRIHHLALKHKAKIVYELEVSRFEEGKQKIKKVYATLKKADVYSEIDADKNLQTTVWGKLAVASEISASYTSQFCGACKKLWRAEMQVDETITTQELIGTVRVIKGGTLIDAIKDFMRPPIFDENDTPFPKYRDFCDKHHISKKMRGNSCLFICPFCRANADADIQASQTIALLRYVKEEKKVEDYFERFRKLKNIKVLGQMKKI

SEQ ID NO: 212 - DNA, 184 bp, synthetic construct
  source 1..184: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggccggatgatcctgacgacggagaccgccgtcgtcgacaagccggcctgagctgcgagaa

SEQ ID NO: 213 - DNA, 194 bp, synthetic construct
  source 1..194: mol_type = other DNA
gctattctcgcagctcaccagtttgagagctatgctggaaacagcatagcaagttcaaataaggctagtccgttatcaacttgaaaaagtggcaccgagtcggtgcgacgagcgcggcgatatcatcatccatggccggatgatcctgacgacggagaccgccgtcgtcgacaagccggcctgagctgcgagaa

SEQ ID NO: 214 - DNA, 179 bp, synthetic construct
  source 1..179: mol_type = other DNA
gctattctcgcagctcaccagttttagagctagaaatagcaagttaaaataaggctagtccgttatcaacttgaaaaagtggcaccgagtcggtgcgagtcggtgcgacgagcgcggcgatatcatcatccatggcacaattaacatctcaatcaaggtaaatgcttgagctgcgagaa

SEQ ID NO: 215 - DNA, 179 bp, synthetic construct
  source 1..179: mol_type = other DNA
gctattctcgcagctcaccagttttagagctagaaatagcaagttaaaataaggctagtccgttatcaacttgaaaaagtggcaccgagtcggtgcgagtcggtgcgacgagcgcggcgatatcatcatccatggagcatttaccttgattgagatgttaattgtgtgagctgcgagaa

SEQ ID NO: 216 - DNA, 182 bp, synthetic construct
  source 1..182: mol_type = other DNA
gctattctcgcagctcaccagttttagagctagaaatagcaagttaaaataaggctagtccgttatcaacttgaaaaagtggcaccgagtcggtgcgagtcggtgcgacgagcgcggcgatatcatcatccatggcaggtttttgacgaaagtgatccagatgatccagtgagctgcgagaa

SEQ ID NO: 217 - DNA, 182 bp, synthetic construct
  source 1..182: mol_type = other DNA
gctattctcgcagctcaccagttttagagctagaaatagcaagttaaaataaggctagtccgttatcaacttgaaaaagtggcaccgagtcggtgcgagtcggtgcgacgagcgcggcgatatcatcatccatggctggatcatctggatcactttcgtcaaaaacctgtgagctgcgagaa

SEQ ID NO: 218 - DNA, 96 bp, synthetic construct
  source 1..96: mol_type = other DNA
gaagccggccttgcacatgcgttttagagctagaaatagcaagttaaaataaggctagtccgttatcaacttgaaaaagtggcaccgagtcggtgc

SEQ ID NO: 219 - DNA, 164 bp, synthetic construct
  source 1..164: mol_type = other DNA
gaagccggccttgcacatgcgttttagagctagaaatagcaagttaaaataaggctagtccgttatcaacttgaaaaagtggcaccgagtcggtgcatatcatcatccatggtaccgttcgtatagcatacattatacgaagttattgagctgcgagaatagcc

SEQ ID NO: 220 - DNA, 172 bp, synthetic construct
  source 1..172: mol_type = other DNA
gaagccggccttgcacatgcgttttagagctagaaatagcaagttaaaataaggctagtccgttatcaacttgaaaaagtggcaccgagtcggtgcgacgagcgcggcgatatcatcatccatggtaccgttcgtatagcatacattatacgaagttattgagctgcgagaa

SEQ ID NO: 221 - DNA, 189 bp, synthetic construct
  source 1..189: mol_type = other DNA
gctattctcgcagctcaccagttttagagctagaaatagcaagttaaaataaggctagtccgttatcaacttgaaaaagtggcaccgagtcggtgctcgacgacgagcgcggcgatatcatcatccatggccggatgatcctgacgacggagaccgccgtcgtcgacaagccggcctgagctgcgagaa

SEQ ID NO: 222 - DNA, 181 bp, synthetic construct
  source 1..181: mol_type = other DNA
gctattctcgcagctcaccagttttagagctagaaatagcaagttaaaataaggctagtccgttatcaacttgaaaaagtggcaccgagtcggtgcgagcgcggcgatatcatcatccatggccggatgatcctgacgacggagaccgccgtcgtcgacaagccggcctgagctgcgagaa

SEQ ID NO: 223 - DNA, 178 bp, synthetic construct
  source 1..178: mol_type = other DNA
gctattctcgcagctcaccagttttagagctagaaatagcaagttaaaataaggctagtccgttatcaacttgaaaaagtggcaccgagtcggtgccgcggcgatatcatcatccatggccggatgatcctgacgacggagaccgccgtcgtcgacaagccggcctgagctgcgagaa

SEQ ID NO: 224 - DNA, 175 bp, synthetic construct
  source 1..175: mol_type = other DNA
gctattctcgcagctcaccagttttagagctagaaatagcaagttaaaataaggctagtccgttatcaacttgaaaaagtggcaccgagtcggtgcggcgatatcatcatccatggccggatgatcctgacgacggagaccgccgtcgtcgacaagccggcctgagctgcgagaa

SEQ ID NO: 225 - DNA, 171 bp, synthetic construct
  source 1..171: mol_type = other DNA
gctattctcgcagctcaccagttttagagctagaaatagcaagttaaaataaggctagtccgttatcaacttgaaaaagtggcaccgagtcggtgcatatcatcatccatggccggatgatcctgacgacggagaccgccgtcgtcgacaagccggcctgagctgcgagaa

SEQ ID NO: 226 - DNA, 194 bp, synthetic construct
  source 1..194: mol_type = other DNA
gctattctcgcagctcaccagttttagagctagaaatagcaagttaaaataaggctagtccgttatcaacttgaaaaagtggcaccgagtcggtgctcgacgacgagcgcggcgatatcatcatccatggccggatgatcctgacgacggagaccgccgtcgtcgacaagccggcctgagctgcgagaatagcc

SEQ ID NO: 227 - DNA, 189 bp, synthetic construct
  source 1..189: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggccggatgatcctgacgacggagaccgccgtcgtcgacaagccggcctgagctgcgagaatagcc

SEQ ID NO: 228 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcatatcatcatccatggccggatgatcctgacgacggagaccgccgtcgtcgacaagccggcctgagctgcgagaatagcc

SEQ ID NO: 229 - DNA, 194 bp, synthetic construct
  source 1..194: mol_type = other DNA
gctgtctccgccgcccgccagttttagagctagaaatagcaagttaaaataaggctagtccgttatcaacttgaaaaagtggcaccgagtcggtgcctgcccatccgcggcggcacgggggtcgcagtcgccatgccggatgatcctgacgacggagaccgccgtcgtcgacaagccggcccgggcggcggaga

SEQ ID NO: 230 - DNA, 189 bp, synthetic construct
  source 1..189: mol_type = other DNA
gctgtctccgccgcccgccagttttagagctagaaatagcaagttaaaataaggctagtccgttatcaacttgaaaaagtggcaccgagtcggtgccatccgcggcggcacgggggtcgcagtcgccatgccggatgatcctgacgacggagaccgccgtcgtcgacaagccggcccgggcggcggaga

SEQ ID NO: 231 - DNA, 184 bp, synthetic construct
  source 1..184: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgccggatgatcctgacgacggagaccgccgtcgtcgacaagccggcccgggcggcggaga

SEQ ID NO: 232 - DNA, 179 bp, synthetic construct
  source 1..179: mol_type = other DNA
gctgtctccgccgcccgccagttttagagctagaaatagcaagttaaaataaggctagtccgttatcaacttgaaaaagtggcaccgagtcggtgcggcacgggggtcgcagtcgccatgccggatgatcctgacgacggagaccgccgtcgtcgacaagccggcccgggcggcggaga

SEQ ID NO: 233 - DNA, 174 bp, synthetic construct
  source 1..174: mol_type = other DNA
gctgtctccgccgcccgccagttttagagctagaaatagcaagttaaaataaggctagtccgttatcaacttgaaaaagtggcaccgagtcggtgcgggggtcgcagtcgccatgccggatgatcctgacgacggagaccgccgtcgtcgacaagccggcccgggcggcggaga

SEQ ID NO: 234 - DNA, 199 bp, synthetic construct
  source 1..199: mol_type = other DNA
gctgtctccgccgcccgccagttttagagctagaaatagcaagttaaaataaggctagtccgttatcaacttgaaaaagtggcaccgagtcggtgcctgcccatccgcggcggcacgggggtcgcagtcgccatgccggatgatcctgacgacggagaccgccgtcgtcgacaagccggcccgggcggcggagacagcg

SEQ ID NO: 235 - DNA, 194 bp, synthetic construct
  source 1..194: mol_type = other DNA
gctgtctccgccgcccgccagttttagagctagaaatagcaagttaaaataaggctagtccgttatcaacttgaaaaagtggcaccgagtcggtgccatccgcggcggcacgggggtcgcagtcgccatgccggatgatcctgacgacggagaccgccgtcgtcgacaagccggcccgggcggcggagacagcg

SEQ ID NO: 236 - DNA, 189 bp, synthetic construct
  source 1..189: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgccggatgatcctgacgacggagaccgccgtcgtcgacaagccggcccgggcggcggagacagcg

SEQ ID NO: 237 - DNA, 184 bp, synthetic construct
  source 1..184: mol_type = other DNA
gctgtctccgccgcccgccagttttagagctagaaatagcaagttaaaataaggctagtccgttatcaacttgaaaaagtggcaccgagtcggtgcggcacgggggtcgcagtcgccatgccggatgatcctgacgacggagaccgccgtcgtcgacaagccggcccgggcggcggagacagcg

SEQ ID NO: 238 - DNA, 179 bp, synthetic construct
  source 1..179: mol_type = other DNA
gctgtctccgccgcccgccagttttagagctagaaatagcaagttaaaataaggctagtccgttatcaacttgaaaaagtggcaccgagtcggtgcgggggtcgcagtcgccatgccggatgatcctgacgacggagaccgccgtcgtcgacaagccggcccgggcggcggagacagcg

SEQ ID NO: 239 - DNA, 96 bp, synthetic construct
  source 1..96: mol_type = other DNA
gcgtggtggggccgccagcggttttagagctagaaatagcaagttaaaataaggctagtccgttatcaacttgaaaaagtggcaccgagtcggtgc

SEQ ID NO: 240 - DNA, 180 bp, synthetic construct
  source 1..180: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggggatgatcctgacgacggagaccgccgtcgtcgacaagccggtgagctgcgagaa

SEQ ID NO: 241 - DNA, 178 bp, synthetic construct
  source 1..178: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggatgatcctgacgacggagaccgccgtcgtcgacaagccgtgagctgcgagaa

SEQ ID NO: 242 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggatgatcctgacgacggagaccgccgtcgtcgacaagcctgagctgcgagaa

SEQ ID NO: 243 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgatcctgacgacggagaccgccgtcgtcgacaagctgagctgcgagaa

SEQ ID NO: 244 - DNA, 182 bp, synthetic construct
  source 1..182: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgcggatgatcctgacgacggagaccgccgtcgtcgacaagccggccgggcggcggaga

SEQ ID NO: 245 - DNA, 180 bp, synthetic construct
  source 1..180: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgggatgatcctgacgacggagaccgccgtcgtcgacaagccggcgggcggcggaga

SEQ ID NO: 246 - DNA, 178 bp, synthetic construct
  source 1..178: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatggatgatcctgacgacggagaccgccgtcgtcgacaagccgcgggcggcggaga

SEQ ID NO: 247 - DNA, 176 bp, synthetic construct
  source 1..176: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgatgatcctgacgacggagaccgccgtcgtcgacaagcccgggcggcggaga

SEQ ID NO: 248 - DNA, 189 bp, synthetic construct
  source 1..189: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccccggatgatcctgacgacggagaccgccgtcgtcgacaagccggcctcctccaggcaatacgcg

SEQ ID NO: 249 - DNA, 184 bp, synthetic construct
  source 1..184: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccccggatgatcctgacgacggagaccgccgtcgtcgacaagccggcctcctccaggcaat

SEQ ID NO: 250 - DNA, 182 bp, synthetic construct
  source 1..182: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgcccggatgatcctgacgacggagaccgccgtcgtcgacaagccggctcctccaggcaat

SEQ ID NO: 251 - DNA, 180 bp, synthetic construct
  source 1..180: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccggatgatcctgacgacggagaccgccgtcgtcgacaagccggtcctccaggcaat

SEQ ID NO: 252 - DNA, 178 bp, synthetic construct
  source 1..178: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccgatgatcctgacgacggagaccgccgtcgtcgacaagccgtcctccaggcaat

SEQ ID NO: 253 - DNA, 176 bp, synthetic construct
  source 1..176: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccatgatcctgacgacggagaccgccgtcgtcgacaagcctcctccaggcaat

SEQ ID NO: 254 - DNA, 97 bp, synthetic construct
  source 1..97: mol_type = other DNA
gagccgagcacgaggggatacgttttagagctagaaatagcaagttaaaataaggctagtccgttatcaacttgaaaaagtggcaccgagtcggtgc

SEQ ID NO: 255 - DNA, 167 bp, synthetic construct
  source 1..167: mol_type = other DNA
gctattctcgcagctcaccagttttagagctagaaatagcaagttaaaataaggctagtccgttatcaacttgaaaaagtggcaccgagtcggtgcggcgatatcatcatccatggatgatcctgacgacggagaccgccgtcgtcgacaagcctgagctgcgagaa

SEQ ID NO: 256 - DNA, 162 bp, synthetic construct
  source 1..162: mol_type = other DNA
gctattctcgcagctcaccagttttagagctagaaatagcaagttaaaataaggctagtccgttatcaacttgaaaaagtggcaccgagtcggtgctatcatcatccatggatgatcctgacgacggagaccgccgtcgtcgacaagcctgagctgcgagaa

SEQ ID NO: 257 - DNA, 157 bp, synthetic construct
  source 1..157: mol_type = other DNA
gctattctcgcagctcaccagttttagagctagaaatagcaagttaaaataaggctagtccgttatcaacttgaaaaagtggcaccgagtcggtgctcatccatggatgatcctgacgacggagaccgccgtcgtcgacaagcctgagctgcgagaa

SEQ ID NO: 258 - DNA, 163 bp, synthetic construct
  source 1..163: mol_type = other DNA
gctattctcgcagctcaccagttttagagctagaaatagcaagttaaaataaggctagtccgttatcaacttgaaaaagtggcaccgagtcggtgcggcgatatcatcatccatggatgatcctgacgacggagaccgccgtcgtcgacaagcctgagctgcg

SEQ ID NO: 259 - DNA, 158 bp, synthetic construct
  source 1..158: mol_type = other DNA
gctattctcgcagctcaccagttttagagctagaaatagcaagttaaaataaggctagtccgttatcaacttgaaaaagtggcaccgagtcggtgctatcatcatccatggatgatcctgacgacggagaccgccgtcgtcgacaagcctgagctgcg

SEQ ID NO: 260 - DNA, 153 bp, synthetic construct
  source 1..153: mol_type = other DNA
gctattctcgcagctcaccagttttagagctagaaatagcaagttaaaataaggctagtccgttatcaacttgaaaaagtggcaccgagtcggtgctcatccatggatgatcctgacgacggagaccgccgtcgtcgacaagcctgagctgcg

SEQ ID NO: 261 - DNA, 167 bp, synthetic construct
  source 1..167: mol_type = other DNA
gctgtctccgccgcccgccagttttagagctagaaatagcaagttaaaataaggctagtccgttatcaacttgaaaaagtggcaccgagtcggtgccgggggtcgcagtcgccatgatgatcctgacgacggagaccgccgtcgtcgacaagcccgggcggcggaga

SEQ ID NO: 262 - DNA, 162 bp, synthetic construct
  source 1..162: mol_type = other DNA
gctgtctccgccgcccgccagttttagagctagaaatagcaagttaaaataaggctagtccgttatcaacttgaaaaagtggcaccgagtcggtgcgtcgcagtcgccatgatgatcctgacgacggagaccgccgtcgtcgacaagcccgggcggcggaga

SEQ ID NO: 263 - DNA, 157 bp, synthetic construct
  source 1..157: mol_type = other DNA
gctgtctccgccgcccgccagttttagagctagaaatagcaagttaaaataaggctagtccgttatcaacttgaaaaagtggcaccgagtcggtgcagtcgccatgatgatcctgacgacggagaccgccgtcgtcgacaagcccgggcggcggaga

SEQ ID NO: 264 - DNA, 163 bp, synthetic construct
  source 1..163: mol_type = other DNA
gctgtctccgccgcccgccagttttagagctagaaatagcaagttaaaataaggctagtccgttatcaacttgaaaaagtggcaccgagtcggtgccgggggtcgcagtcgccatgatgatcctgacgacggagaccgccgtcgtcgacaagcccgggcggcg

SEQ ID NO: 265 - DNA, 158 bp, synthetic construct
  source 1..158: mol_type = other DNA
gctgtctccgccgcccgccagttttagagctagaaatagcaagttaaaataaggctagtccgttatcaacttgaaaaagtggcaccgagtcggtgcgtcgcagtcgccatgatgatcctgacgacggagaccgccgtcgtcgacaagcccgggcggcg

SEQ ID NO: 266 - DNA, 153 bp, synthetic construct
  source 1..153: mol_type = other DNA
gctgtctccgccgcccgccagttttagagctagaaatagcaagttaaaataaggctagtccgttatcaacttgaaaaagtggcaccgagtcggtgcagtcgccatgatgatcctgacgacggagaccgccgtcgtcgacaagcccgggcggcg

SEQ ID NO: 267 - DNA, 180 bp, synthetic construct
  source 1..180: mol_type = other DNA
gagaagcggcgtccggggctagttttagagctagaaatagcaagttaaaataaggctagtccgttatcaacttgaaaaagtggcaccgagtcggtgctctttgtccagagtcacagccataccggatgatcctgacgacggagaccgccgtcgtcgacaagccggccccccggacgccgc

SEQ ID NO: 268 - DNA, 179 bp, synthetic construct
  source 1..179: mol_type = other DNA
gggcacggggccatgtacaagttttagagctagaaatagcaagttaaaataaggctagtccgttatcaacttgaaaaagtggcaccgagtcggtgcggcgtcggcagcccgatcccgttgccggatgatcctgacgacggagaccgccgtcgtcgacaagccggcctacatggccccgt

SEQ ID NO: 269 - DNA, 185 bp, synthetic construct
  source 1..185: mol_type = other DNA
gtgtcaggtggggcggggctagttttagagctagaaatagcaagttaaaataaggctagtccgttatcaacttgaaaaagtggcaccgagtcggtgcgctggctcctcccctggcaccataccggatgatcctgacgacggagaccgccgtcgtcgacaagccggccccccgccccacctgacac

SEQ ID NO: 270 - DNA, 184 bp, synthetic construct
  source 1..184: mol_type = other DNA
gagtgggtcagacgagcaggagttttagagctagaaatagcaagttaaaataaggctagtccgttatcaacttgaaaaagtggcaccgagtcggtgccgactcctcccccatgcagccctccatcccggatgatcctgacgacggagaccgccgtcgtcgacaagccggcctgctcgtctgacc

SEQ ID NO: 271 - DNA, 97 bp, synthetic construct
  source 1..97: mol_type = other DNA
gcagccacccgctctcggcccgttttagagctagaaatagcaagttaaaataaggctagtccgttatcaacttgaaaaagtggcaccgagtcggtgc

SEQ ID NO: 272 - DNA, 97 bp, synthetic construct
  source 1..97: mol_type = other DNA
gtgtagtcaggccgctcacccgttttagagctagaaatagcaagttaaaataaggctagtccgttatcaacttgaaaaagtggcaccgagtcggtgc

SEQ ID NO: 273 - DNA, 97 bp, synthetic construct
  source 1..97: mol_type = other DNA
gctgacaagtctacggaacctgttttagagctagaaatagcaagttaaaataaggctagtccgttatcaacttgaaaaagtggcaccgagtcggtgc

SEQ ID NO: 274 - DNA, 96 bp, synthetic construct
  source 1..96: mol_type = other DNA
gctcctccagcgccttgaccgttttagagctagaaatagcaagttaaaataaggctagtccgttatcaacttgaaaaagtggcaccgagtcggtgc

SEQ ID NO: 275 - DNA, 96 bp, synthetic construct
  source 1..96: mol_type = other DNA
gctattctcgcagctcaccagttttagagctagaaatagcaagttaaaataaggctagtccgttatcaacttgaaaaagtggcaccgagtcggtgc

SEQ ID NO: 276 - DNA, 96 bp, synthetic construct
  source 1..96: mol_type = other DNA
agaagcggcgtccggggctagttttagagctagaaatagcaagttaaaataaggctagtccgttatcaacttgaaaaagtggcaccgagtcggtgc

SEQ ID NO: 277 - DNA, 96 bp, synthetic construct
  source 1..96: mol_type = other DNA
gggcacggggccatgtacaagttttagagctagaaatagcaagttaaaataaggctagtccgttatcaacttgaaaaagtggcaccgagtcggtgc

SEQ ID NO: 278 - DNA, 96 bp, synthetic construct
  source 1..96: mol_type = other DNA
gcgtattgcctggaggatgggttttagagctagaaatagcaagttaaaataaggctagtccgttatcaacttgaaaaagtggcaccgagtcggtgc

SEQ ID NO: 279 - DNA, 96 bp, synthetic construct
  source 1..96: mol_type = other DNA
tgtcaggtggggcggggctagttttagagctagaaatagcaagttaaaataaggctagtccgttatcaacttgaaaaagtggcaccgagtcggtgc

SEQ ID NO: 280 - DNA, 96 bp, synthetic construct
  source 1..96: mol_type = other DNA
agtgggtcagacgagcaggagttttagagctagaaatagcaagttaaaataaggctagtccgttatcaacttgaaaaagtggcaccgagtcggtgc

SEQ ID NO: 281 - DNA, 96 bp, synthetic construct
  source 1..96: mol_type = other DNA
gctgtctccgccgcccgccagttttagagctagaaatagcaagttaaaataaggctagtccgttatcaacttgaaaaagtggcaccgagtcggtgc

SEQ ID NO: 282 - DNA, 96 bp, synthetic construct
  source 1..96: mol_type = other DNA
ggggtcgcagtcgccatggcgttttagagctagaaatagcaagttaaaataaggctagtccgttatcaacttgaaaaagtggcaccgagtcggtgc

SEQ ID NO: 283 - DNA, 184 bp, synthetic construct
  source 1..184: mol_type = other DNA
  misc_difference 148..149: note = cg, gc, at, ta, gg, tt, ga, ag, cc, tc, ct, aa, tg, gt, ca, or ac
gctattctcgcagctcaccagttttagagctagaaatagcaagttaaaataaggctagtccgttatcaacttgaaaaagtggcaccgagtcggtgcgacgagcgcggcgatatcatcatccatggccggatgatcctgacgacggagnncgccgtcgtcgacaagccggcctgagctgcgagaa

SEQ ID NO: 284 - DNA, 183 bp, synthetic construct
  source 1..183: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgccggatgatcctgacgacggagaccgccgtcgtcgacaagccggcctgagctgcgagaa

SEQ ID NO: 285 - DNA, 183 bp, synthetic construct
  source 1..183: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgccggatgatcctgacgacggagagcgccgtcgtcgacaagccggcctgagctgcgagaa

SEQ ID NO: 286 - DNA, 189 bp, synthetic construct
  source 1..189: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccccggatgatcctgacgacggagtccgccgtcgtcgacaagccggcctcctccaggcaatacgcg

SEQ ID NO: 287 - DNA, 189 bp, synthetic construct
  source 1..189: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgccggatgatcctgacgacggagctcgccgtcgtcgacaagccggcccgggcggcggagacagcg

SEQ ID NO: 288 - DNA, 96 bp, synthetic construct
  source 1..96: mol_type = other DNA
gtcacctccaatgactaggggttttagagctagaaatagcaagttaaaataaggctagtccgttatcaacttgaaaaagtggcaccgagtcggtgc

SEQ ID NO: 289 - DNA, 96 bp, synthetic construct
  source 1..96: mol_type = other DNA
gggcaaccacaaacccacgagttttagagctagaaatagcaagttaaaataaggctagtccgttatcaacttgaaaaagtggcaccgagtcggtgc

SEQ ID NO: 290 - DNA, 194 bp, synthetic construct
  source 1..194: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggctatgccggatgatcctgacgacggagtccgccgtcgtcgacaagccggccctagctgagctgcgagaa

SEQ ID NO: 291 - DNA, 189 bp, synthetic construct
  source 1..189: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgccggatgatcctgacgacggagtccgccgtcgtcgacaagccggccctatgagctgcgagaa

SEQ ID NO: 292 - DNA, 184 bp, synthetic construct
  source 1..184: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggccggatgatcctgacgacggagtccgccgtcgtcgacaagccggcctgagctgcgagaa

SEQ ID NO: 293 - DNA, 179 bp, synthetic construct
  source 1..179: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggggatgatcctgacgacggagtccgccgtcgtcgacaagccgtgagctgcgagaa

SEQ ID NO: 294 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgatcctgacgacggagtccgccgtcgtcgacaagctgagctgcgagaa

SEQ ID NO: 295 - DNA, 169 bp, synthetic construct
  source 1..169: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggatcctgacgacggagtccgccgtcgtcgacatgagctgcgagaa

SEQ ID NO: 296 - DNA, 164 bp, synthetic construct
  source 1..164: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcctgacgacggagtccgccgtcgtcgtgagctgcgagaa

SEQ ID NO: 297 - DNA, 159 bp, synthetic construct
  source 1..159: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgacgacggagtccgccgtcgtgagctgcgagaa

SEQ ID NO: 298 - DNA, 154 bp, synthetic construct
  source 1..154: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggacgacggagtccgccgtgagctgcgagaa

SEQ ID NO: 299 - DNA, 149 bp, synthetic construct
  source 1..149: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggacggagtccgtgagctgcgagaa

SEQ ID NO: 300 - DNA, 144 bp, synthetic construct
  source 1..144: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcggagttgagctgcgagaa

SEQ ID NO: 301 - DNA, 182 bp, synthetic construct
  source 1..182: mol_type = other DNA
gaagccggccttgcacatgcgttttagagctagaaatagcaagttaaaataaggctagtccgttatcaacttgaaaaagtggcaccgagtcggtgctcgacgacgagcgcggcgatatcatcatccatggtaccgttcgtatagcatacattatacgaagttattgagctgcgagaatagcc

SEQ ID NO: 302 - DNA, 177 bp, synthetic construct
  source 1..177: mol_type = other DNA
gaagccggccttgcacatgcgttttagagctagaaatagcaagttaaaataaggctagtccgttatcaacttgaaaaagtggcaccgagtcggtgcgacgagcgcggcgatatcatcatccatggtaccgttcgtatagcatacattatacgaagttattgagctgcgagaatagcc

SEQ ID NO: 303 - DNA, 177 bp, synthetic construct
  source 1..177: mol_type = other DNA
gaagccggccttgcacatgcgttttagagctagaaatagcaagttaaaataaggctagtccgttatcaacttgaaaaagtggcaccgagtcggtgctcgacgacgagcgcggcgatatcatcatccatggtaccgttcgtatagcatacattatacgaagttattgagctgcgagaa

SEQ ID NO: 304 - DNA, 159 bp, synthetic construct
  source 1..159: mol_type = other DNA
gaagccggccttgcacatgcgttttagagctagaaatagcaagttaaaataaggctagtccgttatcaacttgaaaaagtggcaccgagtcggtgcatatcatcatccatggtaccgttcgtatagcatacattatacgaagttattgagctgcgagaa

SEQ ID NO: 305 - DNA, 96 bp, synthetic construct
  source 1..96: mol_type = other DNA
ccccacgatggaggggaagagttttagagctagaaatagcaagttaaaataaggctagtccgttatcaacttgaaaaagtggcaccgagtcggtgc

SEQ ID NO: 306 - DNA, 96 bp, synthetic construct
  source 1..96: mol_type = other DNA
ccttctcctggagccgcgacgttttagagctagaaatagcaagttaaaataaggctagtccgttatcaacttgaaaaagtggcaccgagtcggtgc

SEQ ID NO: 307 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcattatatgttcttacagtatggcggcccggattgtaaaaacatataatgtgagctgcgagaa

SEQ ID NO: 308 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcgttatagggtattacagtatggcggtcggtactgcaataccctataacgtgagctgcgagaa

SEQ ID NO: 309 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgtatcattttcatatagttagcacctgcacactatatgaaaatgatacatgagctgcgagaa

SEQ ID NO: 310 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgtctactatctgtatatgcgacacatgtggcataaagacatagtagacatgagctgcgagaa

SEQ ID NO: 311 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcatcgaccctgacgcatgcggaggcggcgctccatgcgtctgacctcatttgagctgcgagaa

SEQ ID NO: 312 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggttagtacccaaatgacaaaaggtcatccttttatcatttgggtactaactgagctgcgagaa

SEQ ID NO: 313 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcttattaaaacccgttccgcttctgtcaaagcggcatcggttttataaactgagctgcgagaa

SEQ ID NO: 314 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggggcgtgatggtcgtgaacctcaacatgacgacgaacacgacctcgcggcctgagctgcgagaa

SEQ ID NO: 315 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtctacatcttgaatatatcaagttataactttgaattatatcagtttatatgagctgcgagaa

SEQ ID NO: 316 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggaattatatctaaaagcactaagctccgccatactgcttttagatataatatgagctgcgagaa

SEQ ID NO: 317 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggatatggggaagtgaatcagtacaaccgccacagtacctgagctgcgagaa

SEQ ID NO: 318 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggggtactgtggcggttgtactgattcacttccccatatctgagctgcgagaa

SEQ ID NO: 319 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgggtggtacaggtgccacattagttgtaccatttatgtgagctgcgagaa

SEQ ID NO: 320 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcataaatggtacaactaatgtggcacctgtaccacccatgagctgcgagaa

SEQ ID NO: 321 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggttgtttttccagatccagttggtcctgtaaatataagtgagctgcgagaa

SEQ ID NO: 322 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcttatatttacaggaccaactggatctggaaaaacaactgagctgcgagaa

SEQ ID NO: 323 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggtactgtggcggttgtactgattcacttccccatattgagctgcgagaa

SEQ ID NO: 324 - DNA, 172 bp, synthetic construct
  source 1..172: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtactgtggcggttgtactgattcacttccccatatgagctgcgagaa

SEQ ID NO: 325 - DNA, 170 bp, synthetic construct
  source 1..170: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggactgtggcggttgtactgattcacttccccattgagctgcgagaa

SEQ ID NO: 326 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggatatggggaagtgaatcagtacaaccgccacagtactgagctgcgagaa

SEQ ID NO: 327 - DNA, 172 bp, synthetic construct
  source 1..172: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtatggggaagtgaatcagtacaaccgccacagtatgagctgcgagaa

SEQ ID NO: 328 - DNA, 170 bp, synthetic construct
  source 1..170: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggatggggaagtgaatcagtacaaccgccacagttgagctgcgagaa

SEQ ID NO: 329 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggataaatggtacaactaatgtggcacctgtaccaccctgagctgcgagaa

SEQ ID NO: 330 - DNA, 172 bp, synthetic construct
  source 1..172: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtaaatggtacaactaatgtggcacctgtaccacctgagctgcgagaa

SEQ ID NO: 331 - DNA, 170 bp, synthetic construct
  source 1..170: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggaaatggtacaactaatgtggcacctgtaccactgagctgcgagaa

SEQ ID NO: 332 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggggtggtacaggtgccacattagttgtaccatttattgagctgcgagaa

SEQ ID NO: 333 - DNA, 172 bp, synthetic construct
  source 1..172: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggggtggtacaggtgccacattagttgtaccatttatgagctgcgagaa

SEQ ID NO: 334 - DNA, 170 bp, synthetic construct
  source 1..170: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggtggtacaggtgccacattagttgtaccattttgagctgcgagaa

SEQ ID NO: 335 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggttatatttacaggaccaactggatctggaaaaacaatgagctgcgagaa

SEQ ID NO: 336 - DNA, 172 bp, synthetic construct
  source 1..172: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtatatttacaggaccaactggatctggaaaaacatgagctgcgagaa

SEQ ID NO: 337 - DNA, 170 bp, synthetic construct
  source 1..170: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggatatttacaggaccaactggatctggaaaaactgagctgcgagaa

SEQ ID NO: 338 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggttgtttttccagatccagttggtcctgtaaatataatgagctgcgagaa

SEQ ID NO: 339 - DNA, 172 bp, synthetic construct
  source 1..172: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgtttttccagatccagttggtcctgtaaatatatgagctgcgagaa

SEQ ID NO: 340 - DNA, 170 bp, synthetic construct
  source 1..170: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggtttttccagatccagttggtcctgtaaatattgagctgcgagaa

SEQ ID NO: 341 - DNA, 151 bp, synthetic construct
  source 1..151: mol_type = other DNA
gctgtctccgccgcccgccagttttagagctagaaatagcaagttaaaataaggctagtccgttatcaacttgaaaaagtggcaccgagtcggtgcagtcgccatgatatggggaagtgaatcagtacaaccgccacagtaccgggcggcg

SEQ ID NO: 342 - DNA, 179 bp, synthetic construct
  source 1..179: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccatatggggaagtgaatcagtacaaccgccacagtactcctccaggcaatacgcg

SEQ ID NO: 343 - DNA, 170 bp, synthetic construct
  source 1..170: mol_type = other DNA
gagaagcggcgtccggggctagttttagagctagaaatagcaagttaaaataaggctagtccgttatcaacttgaaaaagtggcaccgagtcggtgctctttgtccagagtcacagccataatatggggaagtgaatcagtacaaccgccacagtacccccggacgccgc

SEQ ID NO: 344 - DNA, 169 bp, synthetic construct
  source 1..169: mol_type = other DNA
gggcacggggccatgtacaagttttagagctagaaatagcaagttaaaataaggctagtccgttatcaacttgaaaaagtggcaccgagtcggtgcggcgtcggcagcccgatcccgttgatatggggaagtgaatcagtacaaccgccacagtactacatggccccgt

SEQ ID NO: 345 - DNA, 175 bp, synthetic construct
  source 1..175: mol_type = other DNA
gtgtcaggtggggcggggctagttttagagctagaaatagcaagttaaaataaggctagtccgttatcaacttgaaaaagtggcaccgagtcggtgcgctggctcctcccctggcaccataatatggggaagtgaatcagtacaaccgccacagtacccccgccccacctgacac

SEQ ID NO: 346 - DNA, 174 bp, synthetic construct
  source 1..174: mol_type = other DNA
gagtgggtcagacgagcaggagttttagagctagaaatagcaagttaaaataaggctagtccgttatcaacttgaaaaagtggcaccgagtcggtgccgactcctcccccatgcagccctccatcatatggggaagtgaatcagtacaaccgccacagtactgctcgtctgacc

SEQ ID NO: 347 - DNA, 161 bp, synthetic construct
  source 1..161: mol_type = other DNA
gctgtctccgccgcccgccagttttagagctagaaatagcaagttaaaataaggctagtccgttatcaacttgaaaaagtggcaccgagtcggtgccgggggtcgcagtcgccatgatatggggaagtgaatcagtacaaccgccacagtaccgggcggcg

SEQ ID NO: 348 - DNA, 165 bp, synthetic construct
  source 1..165: mol_type = other DNA
gggcacggggccatgtacaagttttagagctagaaatagcaagttaaaataaggctagtccgttatcaacttgaaaaagtggcaccgagtcggtgccgggggtcgcagtcgccatgatatggggaagtgaatcagtacaaccgccacagtaccgggcggcggaga

SEQ ID NO: 349 - DNA, 174 bp, synthetic construct
  source 1..174: mol_type = other DNA
gggcacggggccatgtacaagttttagagctagaaatagcaagttaaaataaggctagtccgttatcaacttgaaaaagtggcaccgagtcggtgcgcggcggcacgggggtcgcagtcgccatgatatggggaagtgaatcagtacaaccgccacagtaccgggcggcggaga

SEQ ID NO: 350 - DNA, 174 bp, synthetic construct
  source 1..174: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccatatggggaagtgaatcagtacaaccgccacagtactcctccaggcaat

SEQ ID NO: 351 - DNA, 165 bp, synthetic construct
  source 1..165: mol_type = other DNA
gggcacggggccatgtacaagttttagagctagaaatagcaagttaaaataaggctagtccgttatcaacttgaaaaagtggcaccgagtcggtgcggcgaatgccggcgtccgccatatggggaagtgaatcagtacaaccgccacagtactcctccaggcaat

SEQ ID NO: 352 - DNA, 170 bp, synthetic construct
  source 1..170: mol_type = other DNA
gggcacggggccatgtacaagttttagagctagaaatagcaagttaaaataaggctagtccgttatcaacttgaaaaagtggcaccgagtcggtgcggcgaatgccggcgtccgccatatggggaagtgaatcagtacaaccgccacagtactcctccaggcaatacgcg

SEQ ID NO: 353 - DNA, 165 bp, synthetic construct
  source 1..165: mol_type = other DNA
gggcacggggccatgtacaagttttagagctagaaatagcaagttaaaataaggctagtccgttatcaacttgaaaaagtggcaccgagtcggtgcggcgtcggcagcccgatcccgttgatatggggaagtgaatcagtacaaccgccacagtactacatggcc

SEQ ID NO: 354 - DNA, 151 bp, synthetic construct
  source 1..151: mol_type = other DNA
gggcacggggccatgtacaagttttagagctagaaatagcaagttaaaataaggctagtccgttatcaacttgaaaaagtggcaccgagtcggtgcgatcccgttgatatggggaagtgaatcagtacaaccgccacagtactacatggcc

SEQ ID NO: 355 - DNA, 155 bp, synthetic construct
  source 1..155: mol_type = other DNA
gggcacggggccatgtacaagttttagagctagaaatagcaagttaaaataaggctagtccgttatcaacttgaaaaagtggcaccgagtcggtgcgatcccgttgatatggggaagtgaatcagtacaaccgccacagtactacatggccccgt

SEQ ID NO: 356 - DNA, 169 bp, synthetic construct
  source 1..169: mol_type = other DNA
gctattctcgcagctcaccagttttagagctagaaatagcaagttaaaataaggctagtccgttatcaacttgaaaaagtggcaccgagtcggtgcgcgcggcgatatcatcatccatggatgatcctgacgacggagaccgccgtcgtcgacaagcctgagctgcgag

SEQ ID NO: 357 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgccgatatcatcatccatggcggatgatcctgacgacggagaccgccgtcgtcgacaagccggctgagctgcgagaatag

SEQ ID NO: 358 - DNA, 168 bp, synthetic construct
  source 1..168: mol_type = other DNA
gctgtctccgccgcccgccagttttagagctagaaatagcaagttaaaataaggctagtccgttatcaacttgaaaaagtggcaccgagtcggtgcgcggcacgggggtcgcagtcgccatgatgatcctgacgacggagaccgccgtcgtcgacaagcccgggcggc

SEQ ID NO: 359 - DNA, 171 bp, synthetic construct
  source 1..171: mol_type = other DNA
gcgtattgcctggaggatgggttttagagctagaaatagcaagttaaaataaggctagtccgttatcaacttgaaaaagtggcaccgagtcggtgcaatgccggcgtccgcccggatgatcctgacgacggagaccgccgtcgtcgacaagccggctcctccaggcaatac

SEQ ID NO: 360 - DNA, 158 bp, synthetic construct
  source 1..158: mol_type = other DNA
gcgtattgcctggaggatgggttttagagctagaaatagcaagttaaaataaggctagtccgttatcaacttgaaaaagtggcaccgagtcggtgcggcgtccgccatgatcctgacgacggagaccgccgtcgtcgacaagcctcctccaggcaata

SEQ ID NO: 361 - DNA, 179 bp, synthetic construct
  source 1..179: mol_type = other DNA
gggaaatgcatcttgcacaagttttagagctagaaatagcaagttaaaataaggctagtccgttatcaacttgaaaaagtggcaccgagtcggtgcagcccctccatgctctctagctgttgccattgggcttgtcgacgacggcggtctccgtcgtcaggatcattgcaagatgcatt

SEQ ID NO: 362 - DNA, 153 bp, synthetic construct
  source 1..153: mol_type = other DNA
gtgtcaggtggggcggggctagttttagagctagaaatagcaagttaaaataaggctagtccgttatcaacttgaaaaagtggcaccgagtcggtgctggcaccataatgatcctgacgacggagaccgccgtcgtcgacaagccccccgccc

SEQ ID NO: 363 - DNA, 96 bp, synthetic construct
  source 1..96: mol_type = other DNA
gtggggacagccccgtctctgttttagagctagaaatagcaagttaaaataaggctagtccgttatcaacttgaaaaagtggcaccgagtcggtgc

SEQ ID NO: 364 - DNA, 97 bp, synthetic construct
  source 1..97: mol_type = other DNA
gctcttgggaaaaaaaccctagttttagagctagaaatagcaagttaaaataaggctagtccgttatcaacttgaaaaagtggcaccgagtcggtgc

SEQ ID NO: 365 - DNA, 97 bp, synthetic construct
  source 1..97: mol_type = other DNA
gtcttgggaaaaaaaccctaagttttagagctagaaatagcaagttaaaataaggctagtccgttatcaacttgaaaaagtggcaccgagtcggtgc

SEQ ID NO: 366 - DNA, 96 bp, synthetic construct
  source 1..96: mol_type = other DNA
gaaaaaaaccctaagggctggttttagagctagaaatagcaagttaaaataaggctagtccgttatcaacttgaaaaagtggcaccgagtcggtgc

SEQ ID NO: 367 - DNA, 97 bp, synthetic construct
  source 1..97: mol_type = other DNA
gctgaggatccttgtgagtgtgttttagagctagaaatagcaagttaaaataaggctagtccgttatcaacttgaaaaagtggcaccgagtcggtgc

SEQ ID NO: 368 - DNA, 97 bp, synthetic construct
  source 1..97: mol_type = other DNA
gtgaggatccttgtgagtgttgttttagagctagaaatagcaagttaaaataaggctagtccgttatcaacttgaaaaagtggcaccgagtcggtgc

SEQ ID NO: 369 - DNA, 96 bp, synthetic construct
  source 1..96: mol_type = other DNA
ggatccttgtgagtgttggggttttagagctagaaatagcaagttaaaataaggctagtccgttatcaacttgaaaaagtggcaccgagtcggtgc

SEQ ID NO: 370 - DNA, 96 bp, synthetic construct
  source 1..96: mol_type = other DNA
gatccttgtgagtgttgggtgttttagagctagaaatagcaagttaaaataaggctagtccgttatcaacttgaaaaagtggcaccgagtcggtgc

SEQ ID NO: 371 - DNA, 96 bp, synthetic construct
  source 1..96: mol_type = other DNA
gttgggtgggaacagctcccgttttagagctagaaatagcaagttaaaataaggctagtccgttatcaacttgaaaaagtggcaccgagtcggtgc

SEQ ID NO: 372 - DNA, 96 bp, synthetic construct
  source 1..96: mol_type = other DNA
gggtgggaacagctcccagggttttagagctagaaatagcaagttaaaataaggctagtccgttatcaacttgaaaaagtggcaccgagtcggtgc

SEQ ID NO: 373 - DNA, 97 bp, synthetic construct
  source 1..97: mol_type = other DNA
gcttctgtgcagcagtttcccgttttagagctagaaatagcaagttaaaataaggctagtccgttatcaacttgaaaaagtggcaccgagtcggtgc

SEQ ID NO: 374 - DNA, 97 bp, synthetic construct
  source 1..97: mol_type = other DNA
gtttccctggccactaaataggttttagagctagaaatagcaagttaaaataaggctagtccgttatcaacttgaaaaagtggcaccgagtcggtgc

SEQ ID NO: 375 - DNA, 97 bp, synthetic construct
  source 1..97: mol_type = other DNA
gttccctggccactaaatagtgttttagagctagaaatagcaagttaaaataaggctagtccgttatcaacttgaaaaagtggcaccgagtcggtgc

SEQ ID NO: 376 - DNA, 96 bp, synthetic construct
  source 1..96: mol_type = other DNA
gattagatagaagccctccagttttagagctagaaatagcaagttaaaataaggctagtccgttatcaacttgaaaaagtggcaccgagtcggtgc

SEQ ID NO: 377 - DNA, 97 bp, synthetic construct
  source 1..97: mol_type = other DNA
gattagatagaagccctccaagttttagagctagaaatagcaagttaaaataaggctagtccgttatcaacttgaaaaagtggcaccgagtcggtgc

SEQ ID NO: 378 - protein, 527 aa, unidentified
MEKNRAVLYLRLSKEDVDKVNKGDDSSSIKSQRLLLTDFALERGFKIVGVYSDDDESGLYDDRPDFERMMTDAKLDEFDIIIAKTQSRFSRNMEHIEKYLHHDLPNLGIRFIGAVDGVDTESDENKKSRQINGLVNEWYCEDLSKNIRSAFKAKMKDGQFLGSSCPYGYKKDPQNHNHLVVDDYAAKVVQKIFNLYLEGYGKAKIGSILSSEGILIPTLYKKDILKQNYHNSKALDTTQNWSYQTIHTILNNEVYLGHLIQNKVNTMSYKDKNKRILPKEKWIIVRNTHEPIITEEMFQDVQKLQKNRTRSVENIEPNGLFSGLIFCADCKHAMSRKYARRGEKGFVGYVCKTYKTQGKNFCESHSIDYDELEEAVLFSIKNEARSILQQEEIDELRKVQAYDETKSYYEMQLENIKSRMEKIEKYKKKTYDNYMDDLISRDDYKKYVTEYDKEIGGLKQQQELINSKTDLEKEISTQYDEWVEAFINYVDIDKLTREIVIELIEKIEVNKDGSINIYYKFKNPYIS

SEQ ID NO: 379 - protein, 510 aa, unidentified
MNTVIYARYSAGPRQTDQSIDGQLRVCTEFCKQRGLTVVDTYCDRHISGRTDERPEFQRLIADAKAHKFEAVVVYKTDRFARNKYDSAIYKRELRRNGIQIFYAAEAIPEGPEGIILESLMEGLAEYYSAELAQKIKRGLNESALKCQSLGSGRPLGYTVDEQKHFQIDPESSQAVKTIFEMYIKGESNAAICDYLNARGLRTSQGNLFNKNSINRIIKNRKYIGEYRYNDIVVEGGMPAIISKETFCMAQAEMERRRTHRAPVSPKAEYLLAGKLFCGHCKGPMQGVSGTGKSGNKWYYYYCANTRGKERTCDKKQVSRDRLEKAVVDFTVRYILQENVLEELSKKVYAAQERQNNTASEIAFYEKKLAENKKAIANILRAIESGAMTQALPARLQELENEQTVIQGELSYLKGARLAFTEDQILFALLQHLDPRPGESERDYHRRIITDFVSEVYLYDDRMLIYFNISSADGKLKHADLSAIESGVFDAGLISSSSRASSFSTRCALI

SEQ ID NO: 380 - protein, 482 aa, unidentified
MNEKNLEIGAAYIRVSTDDQTELSPDAQLRVILEAAKKDGIIIPQEFVFMEDRGRSGRRADNRPEFQRMISTARQNPSPFRYLYLWKFSRFARNQEESAFYKGILRKKCGVTIKSVSEPIMEGMFGRLVEMIIEWSDEFYSVNLSGEVLRGMTQKALEHGYQLTPCLGYDAVGHGRPYVINEEQYQIVEFIHRSFFDGKDMTWIAREANRRGYHTRRGNPFDTRAVRIILTNSFYVGLVKWNDVTFQGTHECRESVTSVFSANQERLNRIHRPRGRRQASSCKHWLSGLLKCSICGASLGYNQTKDLTKRGHAFQCWKYTKGIHPGSCSVSSLKAEAAVLESLQMILETGEVEYTYEQREKHLDDNKLTLIQKSLERLDTKELRIREAYESGIDTLDEFKTNKARLQRERDQLMEELEELHSQEEPEDVPGKEILIERIQNVYDLLQSPDVDNDDKGNAVRSIIKKIVYIKESKTFCFYYYV

SEQ ID NO: 381 - protein, 406 aa, unidentified
MERTIKVIQPGTVKIPTKKRVAAYARVSSGKDAMLHSLSAQVSYYSNMIQQKNEWSYVGIYADEAITGTKDRRVEFNRLIQDCTDGKIDMIITKSISRFARNTLTMLEVVRKLKNINVDVYFEKENIHSISGDGELMLTILASFAQEESRSVSENCKWRIRKGFEQGELINLRFLYGYRINKGKIEIYEKEAEIVRMIFDDYLNGEGCTRIGNKLRKMKVNKLRGGMWNSERVVDIIKNEKYTGNALLQKKYVKDHLSKKLVRNKGILTQYYAEGTHPAIIDIKTFEIAQKIMEANRTKFQGKCGSNRYLFTSKIECGICGKNYRHKDREGKSTWVCANHLKYGNSRCIAKPLNEEKLKKLINEALELKYFDEEIFIRNIKRIKVTGNQTIEFILKDGKVIEEGMI

SEQ ID NO: 382 - protein, 401 aa, unidentified
MKKIKIDRAIQERPATRKQTRNEKIRQSLTEHVDVQVIPAITDREGYEKPKLRVCAYCRVSTDMDTQALSYELQVQNYTDYIRGNDEWRFAGIYADRGISGTSLKHRDEFNRMIEDCKAGKIDLIITKAVTRFARNVLDCISTIRMLKQLEHPVAVYFETERINTLDTTSETYLGLISLFAQGESESKSESLKWSYIRRWKRGTGIYPAWSLLGYEMGEDGKWQIVEAEAELVRIIYDMYLNGYSSPQIAEILTRSGVPTATNQTVWSSGGVLGILRNEKYCGNVLCQKTMTVDVFSHKAIKNTGQKTQYFIEGHHDPIILRSDWDRVQQMIDEKYYRKRRGRRTKPRIVLKGCLAGFTQIDLDWDEDDIARIFYSTTPAAEVATPAMADHIEIIKVKGEN

SEQ ID NO: 383 - protein, 476 aa, unidentified
MKTAAAYIRVSTDDQVEYSPDSQIKLIRDYAKRNDYILPDEFIFRDDGISGKSAKHRPEFTKMIALAKSPEHPFDAILVWKFSRFARNQEESIVFKNILRKIGVEVRSVSEPISEDPFGSLVERIIEWTDEYYIINLSGEVKRGMLEKISRGQPVVPPPVGYKMENGQYIPDENAHFIKEIFEAYAAGEGARHIAQRLAAQGCLTKRGNPIDNRFVDYVLHNPVYIGKLRWSVNSHAASSRHYDSADIIVFDGTHEPLISSELWESVQKRLHEVKTLYPKYQRREQPVSFMLKGLVRCSSCGSTLCYCRTSEPSLQCHSYARGSCRQSHSINIATANEAVIKGLQLAVDKLDFAIAPAKPHYSADAPGTNKLLAAEYKKMERIKAAYANGTDTLEEYAANKKKISAEIARLEAELQQESNVKPINKKAFAKRVSEIIKYISDPHNSEAAKNQALRTVISYIIFDRAATTFNIIFHF

SEQ ID NO: 384 - protein, 550 aa, unidentified
MKIAIYARKSKYSPTGESVENQIQLCKEYLQAKYKSETLEIDEYKDEGYSGGNTNRPDFKKLIAQIEDYDMLICYRLDRISRNVADFSSTLTLLQNNKCDFVSIKEQFDTTSPMGRAMIYISSVFAQLERETIAERIRDNMMELAKMGRWLGGTIPMGFDSEPITFIDENMKERSMTKLIPNVEELKVIELIYEKYLQLGSMGKVVTYLLQNNIKTKKGKDFTLGSIKVILTNPIYVKANQEVVNHLKTQGITICGDVDGKKALLTYNKTTGISNDVGTKTIVKDKSEWIAAVANHKGIIPADKWLQAQNIKDKNKDSFPALGRSNTTIASRVLRCDKCESTMGVTHGHINPVTGKKHYYYNCTLKKRSKGVRCDNKPAKAAEVDEAILITLENMFKAKSSIIDNLKAKNKARRIEMISSNRVDVINKIIEDKTKQIDNLVNKLSLDDDLTDILFKKIKGLKAEIKELEDELLTLTSDNIKLNEDEVVLDFTEKLLEKCSIIRTLDILEQQQIVDALIPLVTWNGDTEVLNIYPLGSPELELKEAESKKK

SEQ ID NO: 385 - protein, 493 aa, unidentified
MKEKVSERKTGAIYIRVSTDKQEELSPDAQLRLLLDYAKKDSIDVPKEYIFQDNGISGRKANKRPAFQNMIALAKSKEHPIDTIIVWKFSRFARNQEESIVYKSLLKKNNVDVVSVSEPLIDGPFGSLIERIIEWMDEYYSIRLSGEVMRGMTQNAMRGHYQSDAPIGYTSPGDKKPPVINPDTVQIPLMIKDMFLSGSTQLQIARKLNDSGYRTKRGNLWDARGVRYVLENPFYIGKSRWNYTERGRRLKPADEVIYADGNWEALWDEDTFKEIQKRLALNMRKSKSRDISAAKHWLSGLLICSSCGGTLAFGGAHNMRGFQCWKYSKGFCSESHYISTGPIEKMVLEYLEAVMHSPALSYTVISSSSVDASSKLSDLERQLQKIDAKEKRIKAAYLNEIDTLEEYKANKTALEEERRTVEKEIEELTLSDVKYSKEDLDKKMKQNISDLLRVLRDESADYIQKGNMMRNVVDHIVFNRKNTSLDVFLKLVV

SEQ ID NO: 386 - protein, 404 aa, unidentified
MKITKKQPLRPRGRSEDKRQSTKNVIRDAYINGPQKEVQIIPAKRDMEAETEKKKLRVCAYCRVSTDEDTQASSYELQVQNYTRMIRENPEWEFAGIFADEGISGTSVLHREHFLEMIEKCKAGEIDLIITKQVSRFARNVLDSLNYIFMLRKLDPPVGVYFETEKLNTLDKSSDMVITVLSLVAQSESEQKSNSLKWSFKRRRAQGLGIYPSWALLGYRLDDEKNWEIVEDEADIVRTIYSLYLDGYSSTQIAELLTKSGIPTVKGLSVWSSGSVLGILKNEKFCGDALCQKTVTIDFFTHKSVKNNGIEPQYFVEGHHIPIIEKNDWLLAQQIRKERRYRKRRSTHRKPRIVVKGALSGFMIVDTSWDEEYVDSLLISATQKPEPAPVIAEEDENFIVIEKE

SEQ ID NO: 387 - protein, 498 aa, unidentified
MADIQPVKNGALYIRVSTHLQEELSPDAQKRLLMEYAEAHNIIVLKEHIYIDSGISGRSARQRPQFNNMIAEAKSKEHPFDVILVWKYSRFARNQEESIVYKSMLKRENVDVISVSEPISDDPFGSLIERIIEWMDEYYSIRLSGEVSRGMAENAMRGNYQARPPLGYRIPGYRQTPVIVPEEAELIQLIFDLYTEKKMGIFEIVRYLNEHGYQTGHKKPFQRRSVTYILKNPTYIGKTIWNQHDQDHKLRDKSEWIIADGKHEPIISKEQFDKAQKRIESTYKPAYRKPTSVCHHWLSSLLKCSSCGRTLVVKRTASKKKDRMYVNFQCYGYQKGICNTNQSISAIKLEPVIMHALEDAMTSGKIHFDVLNPTTLDSSQKQQFLTRLNEIEKKEERIKRAYRDGIDTLEEYKENKSIIQTEKEMLLKKIEHIEEPALSPEEAKPIMMDRIKNVYEIITNPDIGMEEKNKAARSIIEKIVFDRATGSVNIFFYLAHCP

SEQ ID NO: 388 - protein, 500 aa, unidentified [sequence omitted]

SEQ ID NO: 389 - protein, 485 aa, unidentified
MTKKVAIYTRVSTTNQAEEGFSIDEQIDRLTKYAEAMGWQVSDTYTDAGFSGAKLERPAMQRLINDIENKAFDTVLVYKLDRLSRSVRDTLYLVKDVFTKNKIDFISLNESIDTSSAMGSLFLTILSAINEFERENIKERMTMGKLGRAKSGKSMMWTKTAFGYYHNRKTGILEIVPLQATIVEQIFTDYLSGISLTKLRDKLNESGHIGKDIPWSYRTLRQTLDNPVYCGYIKFKDSLFEGMHKPIIPYETYLKVQKELEERQQQTYERNNNPRPFQAKYMLSGMARCGYCGAPLKIVLGHKRKDGSRTMKYHCANRFPRKTKGITVYNDNKKCDSGTYDLSNLENTVIDNLIGFQENNDSLLKIINGNNQPILDTSSFKKQISQIDKKIQKNSDLYLNDFITMDELKDRTDSLQAEKKLLKAKISENKFNDSTDVFELVKTQLGSIPINELSYDNKKKIVNNLVSKVDVTADNVDIIFKFQLA

SEQ ID NO: 390 - protein, 594 aa, unidentified
MSPFIAPDVPEHLLDTVRVFLYARQSKGRSDGSDVSTEAQLAAGRALVASRNAQGGARWVVAGEFVDVGRSGWDPNVTRADFERMMGEVRAGEGDVVVVNELSRLTRKGAHDALEIDNELKKHGVRFMSVLEPFLDTSTPIGVAIFALIAALAKQDSDLKAERLKGAKDEIAALGGVHSSSAPFGMRAVRKKVDNLVISVLEPDEDNPDHVELVERMAKMSFEGVSDNAIATTFEKEKIPSPGMAERRATEKRLASIKARRLNGAEKPIMWRAQTVRWILNHPAIGGFAFERVKHGKAHINVIRRDPGGKPLTPHTGILSGSKWLELQEKRSGKNLSDRKPGAEVEPTLLSGWRFLGCRICGGSMGQSQGGRKRNGDLAEGNYMCANPKGHGGLSVKRSELDEFVASKVWARLRTADMEDEHDQAWIAAAAERFALQHDLAGVADERREQQAHLDNVRRSIKDLQADRKAGLYVGREELETWRSTVLQYRSYEAECTTRLAELDEKMNGSTRVPSEWFSGEDPTAEGGIWASWDVYERREFLSFFLDSVMVDRGRHPETKKYIPLKDRVTLKWAELLKEEDEASEATERELAAL

SEQ ID NO: 391 - protein, 528 aa, Bacillus cereus
MYPYDVPDYAGSYRPESLDVCIYLRKSRKDVEEERRAIEEGSSYNALERHRKRLFAIAKAENHNIIDIFEEVASGESIQERPQMQQLLRKLEGNEIDGVLVIDLDRLGRGDMLDAGMIDRAFRYSSTKIITPTDVYDPDDESWELVFGIKSLISRQELKSITKRLQNGRIDSVKEGKHIGKKPPYGYLKDENLRLYPDPEKAWIVKKIFELMCDGKGRQMIAAELDRLGIDPPVTKRGAWDSSTITSIIKNEVYTGVIVWGKFKHKKRNGKYTRHKNPQEKWIMYENAHEPIISKELFDAANEAHSSRHKPAVITSKKLTNPLAGILKCKLCGYTMLIQTRKDRPHNYLRCNNPACKGKQKQSVFNLVEEKLLYSLQQIVDEYQAQKVEEVEIDDSKLISFKEKAIISKEKELKELQAQKGNLHDLLEQGIYTVEIFLERQKNLVERITSIENDIEVLQKEIETEQIKEHNKTEFIPALKTVIESYHKTTNIELKNQLLKTILSTVTYYRHPDWKTNEFEIQVYFKIS

SEQ ID NO: 392 - protein, 486 aa, unidentified
MYPYDVPDYAGSAVGIYIRVSTQEQASEGHSIESQKKKLASYCEIQGWDDYRFYIEEGISGKNTNRPKLKLLMEHIEKGKINILLVYRLDRLTRSVIDLHKLLNFLQEHGCAFKSATETYDTTTANGRMSMGIVSLLAQWETENMSERIKLNLEHKVLVEGERVGAIPYGFDLSDDEKLVKNEKSAILLDMVERVENGWSVNRIVNYLNLTNNDRNWSPNGVLRLLRNPALYGATRWNDKIAENTHEGIISKERFNRLQQILADRSIHHRRDVKGTYIFQGVLRCPVCDQTLSVNRFIKKRKDGTEYCGVLYRCQPCIKQNKYNLAIGEARFLKALNEYMSTVEFQTVEDEVIPKKSEREMLESQLQQIARKREKYQKAWASDLMSDDEFEKLMVETRETYDECKQKLESCEDPIKIDETYLKEIVYMFHQTFNDLESEKQKEFISKFIRTIRYTVKEQQPIRPDKSKTGKGKQKVIITEVEFYQS

SEQ ID NO: 393 - protein, 472 aa, Staphylococcus lugdunensis
MYPYDVPDYAGSKVAIYTRVSSAEQANEGYSIHEQKKKLISYCEIHDWNEYKVFTDAGISGGSMKRPALQKLMKHLSSFDLVLVYKLDRLTRNVRDLLDMLEEFEQYNVSFKSATEVFDTTSAIGKLFITMVGAMAEWERETIRERSLFGSRAAVREGNYIREAPFCYDNIEGKLHPNEYAKVIDLIVSMFKKGISANEIARRLNSSKVHVPNKKSWNRNSLIRLMRSPVLRGHTKYGDMLIENTHEPVLSEHDYNAINNAISSKTHKSKVKHHAIFRGALVCPQCNRRLHLYAGTVKDRKGYKYDVRRYKCETCSKNKDVKNVSFNESEVENKFVNLLKSYELNKFHIRKVEPVKKIEYDIDKINKQKINYTRSWSLGYIEDDEYFELMEEINATKKMIEEQTTENKQSVSKEQIQSINNFILKGWEELTIKDKEELILSTVDKIEFNFIPKDKKHKTNTLDINNIHFKFS

SEQ ID NO: 394 - DNA, 52 bp, unidentified
  source 1..52: mol_type = unassigned DNA
gtggtttgtctggtcaaccaccgcggtctcagtggtgtacggtacaaaccca

SEQ ID NO: 395 - DNA, 52 bp, synthetic construct
  source 1..52: mol_type = other DNA
gtggtttgtctggtcaaccaccgcgcgctcagtggtgtacggtacaaaccca

SEQ ID NO: 396 - DNA, 52 bp, synthetic construct
  source 1..52: mol_type = other DNA
gtggtttgtctggtcaaccaccgcggcctcagtggtgtacggtacaaaccca

SEQ ID NO: 397 - DNA, 52 bp, synthetic construct
  source 1..52: mol_type = other DNA
gtggtttgtctggtcaaccaccgcgatctcagtggtgtacggtacaaaccca

SEQ ID NO: 398 - DNA, 52 bp, synthetic construct
  source 1..52: mol_type = other DNA
gtggtttgtctggtcaaccaccgcgtactcagtggtgtacggtacaaaccca

SEQ ID NO: 399 - DNA, 52 bp, synthetic construct
  source 1..52: mol_type = other DNA
gtggtttgtctggtcaaccaccgcgggctcagtggtgtacggtacaaaccca

SEQ ID NO: 400 - DNA, 52 bp, synthetic construct
  source 1..52: mol_type = other DNA
gtggtttgtctggtcaaccaccgcgttctcagtggtgtacggtacaaaccca

SEQ ID NO: 401 - DNA, 52 bp, synthetic construct
  source 1..52: mol_type = other DNA
gtggtttgtctggtcaaccaccgcggactcagtggtgtacggtacaaaccca

SEQ ID NO: 402 - DNA, 52 bp, synthetic construct
  source 1..52: mol_type = other DNA
gtggtttgtctggtcaaccaccgcgagctcagtggtgtacggtacaaaccca

SEQ ID NO: 403 - DNA, 52 bp, synthetic construct
  source 1..52: mol_type = other DNA
gtggtttgtctggtcaaccaccgcgccctcagtggtgtacggtacaaaccca

SEQ ID NO: 404 - DNA, 52 bp, synthetic construct
  source 1..52: mol_type = other DNA
gtggtttgtctggtcaaccaccgcgtcctcagtggtgtacggtacaaaccca

SEQ ID NO: 405 - DNA, 52 bp, synthetic construct
  source 1..52: mol_type = other DNA
gtggtttgtctggtcaaccaccgcgctctcagtggtgtacggtacaaaccca

SEQ ID NO: 406 - DNA, 52 bp, synthetic construct
  source 1..52: mol_type = other DNA
gtggtttgtctggtcaaccaccgcgaactcagtggtgtacggtacaaaccca

SEQ ID NO: 407 - DNA, 52 bp, synthetic construct
  source 1..52: mol_type = other DNA
gtggtttgtctggtcaaccaccgcgcactcagtggtgtacggtacaaaccca

SEQ ID NO: 408 - DNA, 52 bp, synthetic construct
  source 1..52: mol_type = other DNA
gtggtttgtctggtcaaccaccgcgacctcagtggtgtacggtacaaaccca

SEQ ID NO: 409 - DNA, 52 bp, synthetic construct
  source 1..52: mol_type = other DNA
gtggtttgtctggtcaaccaccgcgtgctcagtggtgtacggtacaaaccca

SEQ ID NO: 410 - DNA, 46 bp, synthetic construct
  source 1..46: mol_type = other DNA
ggccggcttgtcgacgacggcggtctccgtcgtcaggatcatccgg

SEQ ID NO: 411 - DNA, 46 bp, synthetic construct
  source 1..46: mol_type = other DNA
ggccggcttgtcgacgacggcgaactccgtcgtcaggatcatccgg

SEQ ID NO: 412 - DNA, 46 bp, synthetic construct
  source 1..46: mol_type = other DNA
ggccggcttgtcgacgacggcggactccgtcgtcaggatcatccgg

SEQ ID NO: 413 - DNA, 46 bp, synthetic construct
  source 1..46: mol_type = other DNA
ggccggcttgtcgacgacggcgcactccgtcgtcaggatcatccgg

SEQ ID NO: 414 - DNA, 46 bp, synthetic construct
  source 1..46: mol_type = other DNA
ggccggcttgtcgacgacggcgtactccgtcgtcaggatcatccgg

SEQ ID NO: 415 - DNA, 46 bp, synthetic construct
  source 1..46: mol_type = other DNA
ggccggcttgtcgacgacggcgagctccgtcgtcaggatcatccgg

SEQ ID NO: 416 - DNA, 46 bp, synthetic construct
  source 1..46: mol_type = other DNA
ggccggcttgtcgacgacggcgggctccgtcgtcaggatcatccgg

SEQ ID NO: 417 - DNA, 46 bp, synthetic construct
  source 1..46: mol_type = other DNA
ggccggcttgtcgacgacggcgcgctccgtcgtcaggatcatccgg

SEQ ID NO: 418 - DNA, 46 bp, synthetic construct
  source 1..46: mol_type = other DNA
ggccggcttgtcgacgacggcgtgctccgtcgtcaggatcatccgg

SEQ ID NO: 419 - DNA, 46 bp, synthetic construct
  source 1..46: mol_type = other DNA
ggccggcttgtcgacgacggcgacctccgtcgtcaggatcatccgg

SEQ ID NO: 420 - DNA, 46 bp, synthetic construct
  source 1..46: mol_type = other DNA
ggccggcttgtcgacgacggcggcctccgtcgtcaggatcatccgg

SEQ ID NO: 421 - DNA, 46 bp, synthetic construct
  source 1..46: mol_type = other DNA
ggccggcttgtcgacgacggcgccctccgtcgtcaggatcatccgg

SEQ ID NO: 422 - DNA, 46 bp, synthetic construct
  source 1..46: mol_type = other DNA
ggccggcttgtcgacgacggcgtcctccgtcgtcaggatcatccgg

SEQ ID NO: 423 - DNA, 46 bp, synthetic construct
  source 1..46: mol_type = other DNA
ggccggcttgtcgacgacggcgatctccgtcgtcaggatcatccgg

SEQ ID NO: 424 - DNA, 46 bp, synthetic construct
  source 1..46: mol_type = other DNA
ggccggcttgtcgacgacggcgctctccgtcgtcaggatcatccgg

SEQ ID NO: 425 - DNA, 46 bp, synthetic construct
  source 1..46: mol_type = other DNA
ggccggcttgtcgacgacggcgttctccgtcgtcaggatcatccgg

SEQ ID NO: 426 - DNA, 38 bp, synthetic construct
  source 1..38: mol_type = other DNA
ggcttgtcgacgacggcggtctccgtcgtcaggatcat

SEQ ID NO: 427 - DNA, 38 bp, synthetic construct
  source 1..38: mol_type = other DNA
ggcttgtcgacgacggcgaactccgtcgtcaggatcat

SEQ ID NO: 428 - DNA, 38 bp, synthetic construct
  source 1..38: mol_type = other DNA
ggcttgtcgacgacggcggactccgtcgtcaggatcat

SEQ ID NO: 429 - DNA, 38 bp, synthetic construct
  source 1..38: mol_type = other DNA
ggcttgtcgacgacggcgcactccgtcgtcaggatcat

SEQ ID NO: 430 - DNA, 38 bp, synthetic construct
  source 1..38: mol_type = other DNA
ggcttgtcgacgacggcgtactccgtcgtcaggatcat

SEQ ID NO: 431 - DNA, 38 bp, synthetic construct
  source 1..38: mol_type = other DNA
ggcttgtcgacgacggcgagctccgtcgtcaggatcat

SEQ ID NO: 432 - DNA, 38 bp, synthetic construct
  source 1..38: mol_type = other DNA
ggcttgtcgacgacggcgggctccgtcgtcaggatcat

SEQ ID NO: 433 - DNA, 38 bp, synthetic construct
  source 1..38: mol_type = other DNA
ggcttgtcgacgacggcgcgctccgtcgtcaggatcat

SEQ ID NO: 434 - DNA, 38 bp, synthetic construct
  source 1..38: mol_type = other DNA
ggcttgtcgacgacggcgtgctccgtcgtcaggatcat

SEQ ID NO: 435 - DNA, 38 bp, synthetic construct
  source 1..38: mol_type = other DNA
ggcttgtcgacgacggcgacctccgtcgtcaggatcat

SEQ ID NO: 436 - DNA, 38 bp, synthetic construct
  source 1..38: mol_type = other DNA
ggcttgtcgacgacggcggcctccgtcgtcaggatcat

SEQ ID NO: 437 - DNA, 38 bp, synthetic construct
  source 1..38: mol_type = other DNA
ggcttgtcgacgacggcgccctccgtcgtcaggatcat

SEQ ID NO: 438 - DNA, 38 bp, synthetic construct
  source 1..38: mol_type = other DNA
ggcttgtcgacgacggcgtcctccgtcgtcaggatcat

SEQ ID NO: 439 - DNA, 38 bp, synthetic construct
  source 1..38: mol_type = other DNA
ggcttgtcgacgacggcgatctccgtcgtcaggatcat

SEQ ID NO: 440 - DNA, 38 bp, synthetic construct
  source 1..38: mol_type = other DNA
ggcttgtcgacgacggcgctctccgtcgtcaggatcat

SEQ ID NO: 441 - DNA, 38 bp, synthetic construct
  source 1..38: mol_type = other DNA
ggcttgtcgacgacggcgttctccgtcgtcaggatcat

SEQ ID NO: 442 - DNA, 34 bp, synthetic construct
  source 1..34: mol_type = other DNA
taccgttcgtataatgtatgctatacgaagttat

SEQ ID NO: 443 - DNA, 34 bp, synthetic construct
  source 1..34: mol_type = other DNA
ataacttcgtataatgtatgctatacgaacggta

SEQ ID NO: 444 - DNA, 27 bp, synthetic construct
  source 1..27: mol_type = other DNA
tttaccttgattgagatgttaattgtg

SEQ ID NO: 445 - DNA, 50 bp, synthetic construct
  source 1..50: mol_type = other DNA
gcgagtttttatttcgtttatttcaattaaggtaactaaaaaactccttt

SEQ ID NO: 446 - DNA, 34 bp, synthetic construct
  source 1..34: mol_type = other DNA
ctggatcatctggatcactttcgtcaaaaacctg

SEQ ID NO: 447 - DNA, 57 bp, synthetic construct
  source 1..57: mol_type = other DNA
ttcgggtgctgggttgttgtctctggacagtgatccatgggaaactactcagcacca

SEQ ID NO: 448 - DNA, 38 bp, synthetic construct
  source 1..38: mol_type = other DNA
gatatggggaagtgaatcagtacaaccgccacagtacc

SEQ ID NO: 449 - DNA, 38 bp, synthetic construct
  source 1..38: mol_type = other DNA
tgggtggtacaggtgccacattagttgtaccatttatg

SEQ ID NO: 450 - DNA, 38 bp, synthetic construct
  source 1..38: mol_type = other DNA
gttgtttttccagatccagttggtcctgtaaatataag

SEQ ID NO: 451 - DNA, 30 bp, synthetic construct
  source 1..30: mol_type = other DNA
tggggaagtgaatcagtacaaccgccacag

SEQ ID NO: 452 - DNA, 28 bp, synthetic construct
  source 1..28: mol_type = other DNA
ggggaagtgaatcagtacaaccgccaca

SEQ ID NO: 453 - DNA, 26 bp, synthetic construct
  source 1..26: mol_type = other DNA
gggaagtgaatcagtacaaccgccac

SEQ ID NO: 454 - DNA, 30 bp, synthetic construct
  source 1..30: mol_type = other DNA
ctgtggcggttgtactgattcacttcccca

SEQ ID NO: 455 - DNA, 28 bp, synthetic construct
  source 1..28: mol_type = other DNA
tgtggcggttgtactgattcacttcccc

SEQ ID NO: 456 - DNA, 26 bp, synthetic construct
  source 1..26: mol_type = other DNA
gtggcggttgtactgattcacttccc

SEQ ID NO: 457 - DNA, 30 bp, synthetic construct
  source 1..30: mol_type = other DNA
tttttccagatccagttggtcctgtaaata

SEQ ID NO: 458 - DNA, 28 bp, synthetic construct
  source 1..28: mol_type = other DNA
ttttccagatccagttggtcctgtaaat

SEQ ID NO: 459 - DNA, 26 bp, synthetic construct
  source 1..26: mol_type = other DNA
tttccagatccagttggtcctgtaaa

SEQ ID NO: 460 - DNA, 30 bp, synthetic construct
  source 1..30: mol_type = other DNA
tatttacaggaccaactggatctggaaaaa

SEQ ID NO: 461 - DNA, 28 bp, synthetic construct
  source 1..28: mol_type = other DNA
atttacaggaccaactggatctggaaaa

SEQ ID NO: 462 - DNA, 26 bp, synthetic construct
  source 1..26: mol_type = other DNA
tttacaggaccaactggatctggaaa

SEQ ID NO: 463 - DNA, 50 bp, synthetic construct
  source 1..50: mol_type = other DNA
cattatatgtttttacaatccgggccgccatactgtaagaacatataatg

SEQ ID NO: 464 - DNA, 50 bp, synthetic construct
  source 1..50: mol_type = other DNA
cgttatagggtattgcagtaccgaccgccatactgtaataccctataacg

SEQ ID NO: 465 - DNA, 50 bp, synthetic construct
  source 1..50: mol_type = other DNA
tgtatcattttcatatagtgtgcaggtgctaactatatgaaaatgataca

SEQ ID NO: 466 - DNA, 50 bp, synthetic construct
  source 1..50: mol_type = other DNA
tgtctactatgtctttatgccacatgtgtcgcatatacagatagtagaca

SEQ ID NO: 467 - DNA, 50 bp, synthetic construct
  source 1..50: mol_type = other DNA
aatgaggtcagacgcatggagcgccgcctccgcatgcgtcagggtcgatg

SEQ ID NO: 468 - DNA, 50 bp, synthetic construct
  source 1..50: mol_type = other DNA
gttagtacccaaatgataaaaggatgaccttttgtcatttgggtactaac

SEQ ID NO: 469 - DNA, 50 bp, synthetic construct
  source 1..50: mol_type = other DNA
gtttataaaaccgatgccgctttgacagaagcggaacgggttttaataag

SEQ ID NO: 470 - DNA, 50 bp, synthetic construct
  source 1..50: mol_type = other DNA
ggccgcgaggtcgtgttcgtcgtcatgttgaggttcacgaccatcacgcc

SEQ ID NO: 471 - DNA, 50 bp, synthetic construct
  source 1..50: mol_type = other DNA
tataaactgatataattcaaagttataacttgatatattcaagatgtaga

SEQ ID NO: 472 - DNA, 50 bp, synthetic construct
  source 1..50: mol_type = other DNA
tattatatctaaaagcagtatggcggagcttagtgcttttagatataatt

SEQ ID NO: 473 - DNA, 52 bp, unidentified
  source 1..52: mol_type = unassigned DNA
tgggtttgtaccgtacaccactgagaccgcggtggttgaccagacaaaccac

SEQ ID NO: 474 - DNA, 52 bp, synthetic construct
  source 1..52: mol_type = other DNA
tgggtttgtaccgtacaccactgagcgcgcggtggttgaccagacaaaccac

SEQ ID NO: 475 - DNA, 52 bp, synthetic construct
  source 1..52: mol_type = other DNA
tgggtttgtaccgtacaccactgaggccgcggtggttgaccagacaaaccac

SEQ ID NO: 476 - DNA, 52 bp, synthetic construct
  source 1..52: mol_type = other DNA
tgggtttgtaccgtacaccactgagatcgcggtggttgaccagacaaaccac

SEQ ID NO: 477 - DNA, 52 bp, synthetic construct
  source 1..52: mol_type = other DNA
tgggtttgtaccgtacaccactgagtacgcggtggttgaccagacaaaccac

SEQ ID NO: 478 - DNA, 52 bp, synthetic construct
  source 1..52: mol_type = other DNA
tgggtttgtaccgtacaccactgagcccgcggtggttgaccagacaaaccac

SEQ ID NO: 479 - DNA, 52 bp, synthetic construct
  source 1..52: mol_type = other DNA
tgggtttgtaccgtacaccactgagaacgcggtggttgaccagacaaaccac

SEQ ID NO: 480 - DNA, 52 bp, synthetic construct
  source 1..52: mol_type = other DNA
tgggtttgtaccgtacaccactgagtccgcggtggttgaccagacaaaccac

SEQ ID NO: 481 - DNA, 52 bp, synthetic construct
  source 1..52: mol_type = other DNA
tgggtttgtaccgtacaccactgagctcgcggtggttgaccagacaaaccac

SEQ ID NO: 482 - DNA, 52 bp, synthetic construct
  source 1..52: mol_type = other DNA
tgggtttgtaccgtacaccactgagggcgcggtggttgaccagacaaaccac

SEQ ID NO: 483 - DNA, 52 bp, synthetic construct
  source 1..52: mol_type = other DNA
tgggtttgtaccgtacaccactgaggacgcggtggttgaccagacaaaccac

SEQ ID NO: 484 - DNA, 52 bp, synthetic construct
  source 1..52: mol_type = other DNA
tgggtttgtaccgtacaccactgagagcgcggtggttgaccagacaaaccac

SEQ ID NO: 485 - DNA, 52 bp, synthetic construct
  source 1..52: mol_type = other DNA
tgggtttgtaccgtacaccactgagttcgcggtggttgaccagacaaaccac

SEQ ID NO: 486 - DNA, 52 bp, synthetic construct
  source 1..52: mol_type = other DNA
tgggtttgtaccgtacaccactgagtgcgcggtggttgaccagacaaaccac

SEQ ID NO: 487 - DNA, 52 bp, synthetic construct
  source 1..52: mol_type = other DNA
tgggtttgtaccgtacaccactgaggtcgcggtggttgaccagacaaaccac

SEQ ID NO: 488 - DNA, 52 bp, synthetic construct
  source 1..52: mol_type = other DNA
tgggtttgtaccgtacaccactgagcacgcggtggttgaccagacaaaccac

SEQ ID NO: 489 - DNA, 46 bp, synthetic construct
  source 1..46: mol_type = other DNA
ccggatgatcctgacgacggagaccgccgtcgtcgacaagccggcc

SEQ ID NO: 490 - DNA, 46 bp, synthetic construct
  source 1..46: mol_type = other DNA
ccggatgatcctgacgacggagttcgccgtcgtcgacaagccggcc

SEQ ID NO: 491 - DNA, 46 bp, synthetic construct
  source 1..46: mol_type = other DNA
ccggatgatcctgacgacggagtccgccgtcgtcgacaagccggcc

SEQ ID NO: 492 - DNA, 46 bp, synthetic construct
  source 1..46: mol_type = other DNA
ccggatgatcctgacgacggagtgcgccgtcgtcgacaagccggcc

SEQ ID NO: 493 - DNA, 46 bp, synthetic construct
  source 1..46: mol_type = other DNA
ccggatgatcctgacgacggagtacgccgtcgtcgacaagccggcc

SEQ ID NO: 494 - DNA, 46 bp, synthetic construct
  source 1..46: mol_type = other DNA
ccggatgatcctgacgacggagctcgccgtcgtcgacaagccggcc

SEQ ID NO: 495 - DNA, 46 bp, synthetic construct
  source 1..46: mol_type = other DNA
ccggatgatcctgacgacggagcccgccgtcgtcgacaagccggcc

SEQ ID NO: 496 - DNA, 46 bp, synthetic construct
  source 1..46: mol_type = other DNA
ccggatgatcctgacgacggagcgcgccgtcgtcgacaagccggcc

SEQ ID NO: 497 - DNA, 46 bp, synthetic construct
  source 1..46: mol_type = other DNA
ccggatgatcctgacgacggagcacgccgtcgtcgacaagccggcc

SEQ ID NO: 498 - DNA, 46 bp, synthetic construct
  source 1..46: mol_type = other DNA
ccggatgatcctgacgacggaggtcgccgtcgtcgacaagccggcc

SEQ ID NO: 499 - DNA, 46 bp, synthetic construct
  source 1..46: mol_type = other DNA
ccggatgatcctgacgacggaggccgccgtcgtcgacaagccggcc

SEQ ID NO: 500 - DNA, 46 bp, synthetic construct
  source 1..46: mol_type = other DNA
ccggatgatcctgacgacggagggcgccgtcgtcgacaagccggcc

SEQ ID NO: 501 - DNA, 46 bp, synthetic construct
  source 1..46: mol_type = other DNA
ccggatgatcctgacgacggaggacgccgtcgtcgacaagccggcc

SEQ ID NO: 502 - DNA, 46 bp, synthetic construct
  source 1..46: mol_type = other DNA
ccggatgatcctgacgacggagatcgccgtcgtcgacaagccggcc

SEQ ID NO: 503 - DNA, 46 bp, synthetic construct
  source 1..46: mol_type = other DNA
ccggatgatcctgacgacggagagcgccgtcgtcgacaagccggcc

SEQ ID NO: 504 - DNA, 46 bp, synthetic construct
  source 1..46: mol_type = other DNA
ccggatgatcctgacgacggagaacgccgtcgtcgacaagccggcc

SEQ ID NO: 505 - DNA, 38 bp, synthetic construct
  source 1..38: mol_type = other DNA
atgatcctgacgacggagaccgccgtcgtcgacaagcc

SEQ ID NO: 506 - DNA, 38 bp, synthetic construct
  source 1..38: mol_type = other DNA
atgatcctgacgacggagttcgccgtcgtcgacaagcc

SEQ ID NO: 507 - DNA, 38 bp, synthetic construct
  source 1..38: mol_type = other DNA
atgatcctgacgacggagtccgccgtcgtcgacaagcc

SEQ ID NO: 508 - DNA, 38 bp, synthetic construct
  source 1..38: mol_type = other DNA
atgatcctgacgacggagtgcgccgtcgtcgacaagcc

SEQ ID NO: 509 - DNA, 38 bp, synthetic construct
  source 1..38: mol_type = other DNA
atgatcctgacgacggagtacgccgtcgtcgacaagcc

SEQ ID NO: 510 - DNA, 38 bp, synthetic construct
  source 1..38: mol_type = other DNA
atgatcctgacgacggagctcgccgtcgtcgacaagcc

SEQ ID NO: 511 - DNA, 38 bp, synthetic construct
  source 1..38: mol_type = other DNA
atgatcctgacgacggagcccgccgtcgtcgacaagcc

SEQ ID NO: 512 - DNA, 38 bp, synthetic construct
  source 1..38: mol_type = other DNA
atgatcctgacgacggagcgcgccgtcgtcgacaagcc

SEQ ID NO: 513 - DNA, 38 bp, synthetic construct
  source 1..38: mol_type = other DNA
atgatcctgacgacggagcacgccgtcgtcgacaagcc

SEQ ID NO: 514 - DNA, 38 bp, synthetic construct
  source 1..38: mol_type = other DNA
atgatcctgacgacggaggtcgccgtcgtcgacaagcc

SEQ ID NO: 515 - DNA, 38 bp, synthetic construct
  source 1..38: mol_type = other DNA
atgatcctgacgacggaggccgccgtcgtcgacaagcc

SEQ ID NO: 516 - DNA, 38 bp, synthetic construct
  source 1..38: mol_type = other DNA
atgatcctgacgacggagggcgccgtcgtcgacaagcc

SEQ ID NO: 517 - DNA, 38 bp, synthetic construct
  source 1..38: mol_type = other DNA
atgatcctgacgacggaggacgccgtcgtcgacaagcc

SEQ ID NO: 518 - DNA, 38 bp, synthetic construct
  source 1..38: mol_type = other DNA
atgatcctgacgacggagatcgccgtcgtcgacaagcc

SEQ ID NO: 519 - DNA, 38 bp, synthetic construct
  source 1..38: mol_type = other DNA
atgatcctgacgacggagagcgccgtcgtcgacaagcc

SEQ ID NO: 520 - DNA, 38 bp, synthetic construct
  source 1..38: mol_type = other DNA
atgatcctgacgacggagaacgccgtcgtcgacaagcc

SEQ ID NO: 521 - DNA, 34 bp, synthetic construct
  source 1..34: mol_type = other DNA
ataacttcgtatagcatacattatacgaacggta

SEQ ID NO: 522 - DNA, 34 bp, synthetic construct
  source 1..34: mol_type = other DNA
taccgttcgtatagcatacattatacgaagttat

SEQ ID NO: 523 - DNA, 27 bp, synthetic construct
  source 1..27: mol_type = other DNA
cacaattaacatctcaatcaaggtaaa

SEQ ID NO: 524 - DNA, 50 bp, synthetic construct
  source 1..50: mol_type = other DNA
aaaggagttttttagttaccttaattgaaataaacgaaataaaaactcgc

SEQ ID NO: 525 - DNA, 34 bp, synthetic construct
  source 1..34: mol_type = other DNA
caggtttttgacgaaagtgatccagatgatccag

SEQ ID NO: 526 - DNA, 57 bp, synthetic construct
  source 1..57: mol_type = other DNA
tggtgctgagtagtttcccatggatcactgtccagagacaacaacccagcacccgaa

SEQ ID NO: 527 - DNA, 38 bp, synthetic construct
  source 1..38: mol_type = other DNA
ggtactgtggcggttgtactgattcacttccccatatc

SEQ ID NO: 528 - DNA, 38 bp, synthetic construct
  source 1..38: mol_type = other DNA
cataaatggtacaactaatgtggcacctgtaccaccca

SEQ ID NO: 529 - DNA, 38 bp, synthetic construct
  source 1..38: mol_type = other DNA
cttatatttacaggaccaactggatctggaaaaacaac

SEQ ID NO: 530 - DNA, 50 bp, synthetic construct
  source 1..50: mol_type = other DNA
cattatatgttcttacagtatggcggcccggattgtaaaaacatataatg

SEQ ID NO: 531 - DNA, 50 bp, synthetic construct
  source 1..50: mol_type = other DNA
cgttatagggtattacagtatggcggtcggtactgcaataccctataacg

SEQ ID NO: 532 - DNA, 50 bp, synthetic construct
  source 1..50: mol_type = other DNA
tgtatcattttcatatagttagcacctgcacactatatgaaaatgataca

SEQ ID NO: 533 - DNA, 50 bp, synthetic construct
  source 1..50: mol_type = other DNA
tgtctactatctgtatatgcgacacatgtggcataaagacatagtagaca

SEQ ID NO: 534 - DNA, 50 bp, synthetic construct
  source 1..50: mol_type = other DNA
catcgaccctgacgcatgcggaggcggcgctccatgcgtctgacctcatt

SEQ ID NO: 535 - DNA, 50 bp, synthetic construct
  source 1..50: mol_type = other DNA
gttagtacccaaatgacaaaaggtcatccttttatcatttgggtactaac

SEQ ID NO: 536 - DNA, 50 bp, synthetic construct
  source 1..50: mol_type = other DNA
cttattaaaacccgttccgcttctgtcaaagcggcatcggttttataaac

SEQ ID NO: 537 - DNA, 50 bp, synthetic construct
  source 1..50: mol_type = other DNA
ggcgtgatggtcgtgaacctcaacatgacgacgaacacgacctcgcggcc

SEQ ID NO: 538 - DNA, 50 bp, synthetic construct
  source 1..50: mol_type = other DNA
tctacatcttgaatatatcaagttataactttgaattatatcagtttata

SEQ ID NO: 539 - DNA, 50 bp, synthetic construct
  source 1..50: mol_type = other DNA
aattatatctaaaagcactaagctccgccatactgcttttagatataata

SEQ ID NO: 540 - DNA, 52 bp, synthetic construct
  source 1..52: mol_type = other DNA
gtggtttctttgatgaacaactgcagtctcagtggtgtacggtacaaaccca

SEQ ID NO: 541 - DNA, 52 bp, synthetic construct
  source 1..52: mol_type = other DNA
gtggtttgtttggttaaccacagcggtctcagtggtgtacggtacaaaccca

SEQ ID NO: 542 - DNA, 52 bp, synthetic construct
  source 1..52: mol_type = other DNA
gtggtttgtttgatgaaccactgaagtctcagtggtgtacggtacaaaccca

SEQ ID NO: 543 - DNA, 60 bp, synthetic construct
  source 1..60: mol_type = other DNA
ggcggcggcggcagcggcggcggcggcagcggcggcggcggcagcggcggcggcggcagc

SEQ ID NO: 544 - DNA, 24 bp, synthetic construct
  source 1..24: mol_type = other DNA
ggcggcgcgagcccggcgggcggc

SEQ ID NO: 545 - DNA, 36 bp, synthetic construct
  source 1..36: mol_type = other DNA
ggcggcgcgagcccggcggcgccggcgccggcgggc

SEQ ID NO: 546 - DNA, 126 bp, synthetic construct
  source 1..126: mol_type = other DNA
gcggaagcggcgaaagaagcggcgaaagaagcggcgaaagaagcggcgaaagcgctggaagcggaagcggcggcgaaagaagcggcggcgaaagaagcggcggcgaaagaagcggcggcgaaagcg

SEQ ID NO: 547 - DNA, 66 bp, synthetic construct
  source 1..66: mol_type = other DNA
gcgcatcatagcgaagatccgggcggcggcggcagcggcggcggcggcagcggcggcggcggcagc

SEQ ID NO: 548 - DNA, 45 bp, synthetic construct
  source 1..45: mol_type = other DNA
ggcggcggcggcagcgaagcggcggcgaaaggcggcggcggcagc

SEQ ID NO: 549 - DNA, 45 bp, synthetic construct
  source 1..45: mol_type = other DNA
gaagcggcggcgaaaggcggcggcggcagcgaagcggcggcgaaa

SEQ ID NO: 550 - DNA, 102 bp, synthetic construct
  source 1..102: mol_type = other DNA
agcggcggcagcagcggcggcagcagcggcagcgaaaccccgggcaccagcgaaagcgcgaccccggaaagcagcggcggcagcagcggcggcagcagcacc

SEQ ID NO: 551 - protein, 20 aa, synthetic construct
GGGGSGGGGSGGGGSGGGGS

SEQ ID NO: 552 - protein, 8 aa, synthetic construct
GGASPAGG

SEQ ID NO: 553 - protein, 12 aa, synthetic construct
GGASPAAPAPAG

SEQ ID NO: 554 - protein, 42 aa, synthetic construct
AEAAKEAAKEAAKEAAKALEAEAAAKEAAAKEAAAKEAAAKA

SEQ ID NO: 555 - protein, 22 aa, synthetic construct
AHHSEDPGGGGSGGGGSGGGGS

SEQ ID NO: 556 - protein, 15 aa, synthetic construct
GGGGSEAAAKGGGGS

SEQ ID NO: 557 - protein, 15 aa, synthetic construct
EAAAKGGGGSEAAAK

SEQ ID NO: 558 - protein, 34 aa, synthetic construct
SGGSSGGSSGSETPGTSESATPESSGGSSGGSST

SEQ ID NO: 559 - DNA, 148 bp, Synthetic construct
  source 1..148: mol_type = other DNA
gacgcgttttacccggagcagtttaagagctatgctggaaacagcatagcaagtttaaataaggctagtccgttatcaacttgaaaaagtggcaccgagtcggtgcacgacggagaccgccgtcgtcgacaagcctccgggtaaaacg

SEQ ID NO: 560 - DNA, 148 bp, synthetic construct
  source 1..148: mol_type = other DNA
acaaggggataaaggtcgctgtttaagagctatgctggaaacagcatagcaagtttaaataaggctagtccgttatcaacttgaaaaagtggcaccgagtcggtgcacgacggcggtctccgtcgtcaggatcatgacctttatcccc

SEQ ID NO: 561 - DNA, 138 bp, synthetic construct
  source 1..138: mol_type = other DNA
cttgtatgccccgagaagtggttttagagctagaaatagcaagttaaaataaggctagtccgttatcaacttgaaaaagtggcaccgagtcggtgcacgacggagaccgccgtcgtcgacaagccttctcggggcata

SEQ ID NO: 562 - DNA, 138 bp, synthetic construct
  source 1..138: mol_type = other DNA
tatatatacttgctagggctgttttagagctagaaatagcaagttaaaataaggctagtccgttatcaacttgaaaaagtggcaccgagtcggtgcacgacggcggtctccgtcgtcaggatcatcctagcaagtata

SEQ ID NO: 563 - DNA, 4732 bp, synthetic construct
  source 1..4732: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctctcgctattacttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcactgcccgcagatctactagtggcttgtcgacgacggcggtctccgtcgtcaggatcattaggtcagtgaagagaagaacaaaaagcagcatattacagttagttgtcttcatcaatctttaaatatgttgtgtggtttttctctccctgtttccacagttatgggcaacagcttcagcaccagcgccttcggccctgtggccttttctctgggcctcctgctcgtgctgcctgccgcttttccagctcctgtgttcaccctggaagatttcgtgggagattggcggcagaccgccggctacaacctggaccaagtgctggaacagggcggagtgtccagcctgtttcagaacctgggcgtctccgtgacccctatccagcggatcgtgctgagcggcgagaacggcctgaaaatcgacatccatgtgattatcccctacgagggcctgagcggagatcagatgggccagatcgagaaaatcttcaaggtggtgtaccccgtcgacgaccaccacttcaaggtgatcctgcactacggcaccctggtgatcgacggcgttacccctaacatgatcgactacttcggcagaccctatgagggaattgccgtgttcgacggcaagaaaatcaccgtgaccggcacactgtggaacggcaacaagatcatcgatgagcgcctgatcaacccagacggcagcctgctgttcagagtgacaatcaatggcgtgacaggctggagactttgtgaaagaatcctggccggttctggcgagggcagaggatctctgctgacatgcggcgatgtggaagagaatcctggacctgctatgaaaatcgagtgcagaattacaggcacactgaacggagttgaattcgagctggtcggcggaggcgagggcacacctgagcagggcagaatgaccaacaagatgaaaagcaccaagggcgccctgaccttttctccttacctgctgagccacgtgatgggctatggcttctaccacttcggcacctaccccagcggctatgaaaaccccttcctgcatgctatcaacaacggaggctacaccaataccagaatcgagaagtacgaggacggcggcgtgctgcacgtgtccttcagctacagatacgaggccggcagagtgatcggcgacttcaaggtggtgggcacaggatttccagaagatagcgtgatcttcaccgacaagatcatccggagcaacgccaccgtggaacacctgcaccccatgggcgataatgtgctggtgggctcctttgctagaacattctccctgcgggacggcggatactacagcttcgtggtcgacagccacatgcacttcaagtctgccatccacccttctatcctgcagaacggcggacctatgttcgccttccggcgggtggaggaactccacagcaacaccgagctgggcatcgtggaataccagcacgcctttaagacccctatcgccttcgccagaagcagagccaggtgagagtttaaacccgctgatcagcctcgactgtgccttctagttgccagccatctgttgtttgcccctcccccgtgccttccttgaccctggaaggtgccactcccactgtcctttcctaataaaatgagaaaattgcatcgcattgtctgagtaggtgtcattctattctggggggtggggtggggcaggacagcaagggggaggattgggaagacaatagcaggcatgctggggatgcggtgggctctatggactagtagatctcactgcccgcccactccctctctgcgcgctcgctcgctcactgaggccgggcgaccaaaggtcgcccgacgcccgggctttgcccgggcggcctcagtgagcgagcgagcgcgcagagagggatgcattaatgggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 564 - RNA, 131 nt, synthetic construct
  source 1..131: mol_type = other RNA
gacgcgttttacccggagcagttttagagctagaaatagcaagttaaaataaggctagtccgttatcaacttgaaaaagtggcaccgagtcggtgcagaccgccgtcgtcgacaagcctccgggtaaaacg

SEQ ID NO: 565 - RNA, 131 nt, synthetic construct
  source 1..131: mol_type = other RNA
acaaggggataaaggtcgctgttttagagctagaaatagcaagttaaaataaggctagtccgttatcaacttgaaaaagtggcaccgagtcggtgccggtctccgtcgtcaggatcatgacctttatcccc

SEQ ID NO: 566 - RNA, 131 nt, synthetic construct
  source 1..131: mol_type = other RNA
agtgacagtgccaggatcaggttttagagctagaaatagcaagttaaaataaggctagtccgttatcaacttgaaaaagtggcaccgagtcggtgcagaccgccgtcgtcgacaagccatcctggcactgt

SEQ ID NO: 567 - RNA, 131 nt, synthetic construct
  source 1..131: mol_type = other RNA
gttgacatcatgtctggagtgttttagagctagaaatagcaagttaaaataaggctagtccgttatcaacttgaaaaagtggcaccgagtcggtgccggtctccgtcgtcaggatcatccagacatgatgt

SEQ ID NO: 568 - DNA, 86 bp, synthetic construct
  source 1..86: mol_type = other DNA
ataacttcgtataatgtatgctatacgaagttatgtggtttgtctggtcaaccaccgcggtctcagtggtgtacggtacaaaccca

SEQ ID NO: 569 - DNA, 86 bp, synthetic construct
  source 1..86: mol_type = other DNA
tattgaagcatattacatacgatatgcttcaatacaccaaacagaccagttggtggcgccagagtcaccacatgccatgtttgggt

SEQ ID NO: 570 - DNA, 86 bp, synthetic construct
  source 1..86: mol_type = other DNA
gaagttcctattctctagaaagtataggaacttcgtggtttgtctggtcaaccaccgcggtctcagtggtgtacggtacaaaccca

SEQ ID NO: 571 - DNA, 86 bp, synthetic construct
  source 1..86: mol_type = other DNA
cttcaaggataagagatctttcatatccttgaagcaccaaacagaccagttggtggcgccagagtcaccacatgccatgtttgggt

SEQ ID NO: 572 - DNA, 99 bp, synthetic construct
  source 1..99: mol_type = other DNA
tggccggcttgtcgacgacggcgccctccgtcgtcaggatcatccgggtggtttgtctggtcaaccaccgcggtctcagtggtgtacggtacaaaccca

SEQ ID NO: 573 - DNA, 99 bp, synthetic construct
  source 1..99: mol_type = other DNA
accggccgaacagctgctgccgcgggaggcagcagtcctagtaggcccaccaaacagaccagttggtggcgccagagtcaccacatgccatgtttgggt

SEQ ID NO: 574 - DNA, 98 bp, synthetic construct
  source 1..98: mol_type = other DNA
gtggtttgtctggtcaaccaccgcggtctcagtggtgtacggtacaaacccataggtcagtgaagagaagaacaaaaagcagcatattacagttagtt

SEQ ID NO: 575 - DNA, 98 bp, synthetic construct
  source 1..98: mol_type = other DNA
caccaaacagaccagttggtggcgccagagtcaccacatgccatgtttgggtatccagtcacttctcttcttgtttttcgtcgtataatgtcaatcaa

SEQ ID NO: 576 - DNA, 49 bp, synthetic construct
  source 1..49: mol_type = other DNA
gtggtttgtctggtcaaccaccgcgccctccgtcgtcaggatcatccgg

SEQ ID NO: 577 - DNA, 49 bp, synthetic construct
  source 1..49: mol_type = other DNA
caccaaacagaccagttggtggcgcgggaggcagcagtcctagtaggcc

(19) United States
(12) Patent Application Publication
(10) Pub. No.: US 2025/0057980 A1
(43) Pub. Date: Feb. 20, 2025

(54) Co-Delivery of a Gene Editor Construct and a Donor Template

(71) Applicant: TOME BIOSCIENCES, INC., Watertown, MA (US)

(72) Inventors: Jonathan Douglas Finn, Melrose, MA (US); Rahul Kakkar, Subury, MA (US); Xin Jenny Xie, Sudbury, MA (US); Brett Joseph Gordon Estes, Arlington, MA (US)

(21) Appl. No.: 18/721,630

(22) PCT Filed: Dec. 22, 2022
(86) PCT No.: PCT/US2022/082297

§ 371 Date: Jun. 18, 2024

Related U.S. Application Data
(60) Provisional application No. 63/292,698, filed on Dec. 22, 2021.
(60) Provisional application No. 63/318,343, filed on Mar. 9, 2022.
(60) Provisional application No. 63/355,235, filed on Jun. 24, 2022.

(51) Int. Cl. A61K 48/00; C12N 9/12; C12N 9/22; C12N 15/11; C12N 15/86; C12N 15/88; C12N 15/90

(52) U.S. Cl. CPC A61K 48/005; A61K 48/0083; C12N 9/1276; C12N 9/22; C12N 15/111; C12N 15/86; C12N 15/88; C12N 15/907; C12N 2310/20; C12N 2710/10343; C12N 2750/14143; C12N 2800/30; C12N 2800/40; C12Y 207/07049

(57) ABSTRACT

The present disclosure provides compositions, methods, and an overall platform for co-delivery of gene editor polynucleotides and template polynucleotides. The gene editor polynucleotide packaged in a LNP is co-delivered with a template polynucleotide (i.e., “cargo” or “payload”) packaged into a separate vector that is capable of localizing the donor template to a cell nucleus. In certain embodiments, the donor template vector is an AAV, a helper dependent adenovirus, or an integration deficient lentivirus. In typical embodiments, the template polynucleotide is integrated into the genomic integration recognition site by an integrase, optionally by an integrase fused/linked to a gene editor protein.

### 1. CROSS-REFERENCE TO RELATED APPLICATION

This application claims the benefit of U.S. Provisional Application No. 63/292,698, filed Dec. 22, 2021; U.S. Provisional Application No. 63/318,343, filed Mar. 9, 2022; and U.S. Provisional Application No. 63/355,235, filed on Jun. 24, 2022, each of which is hereby incorporated in its entirety by reference.

### 2. SEQUENCE LISTING

The instant application contains a Sequence Listing with 577, which has been submitted via Patent Center and is hereby incorporated by reference in its entirety. Said XML copy, created on Dec. 22, 2022, is named 50699PCT-SequenceListing.xml, and is 780,344 bytes in size.

### 3. BACKGROUND

Programmable, efficient, and multiplexed genome integration of large, diverse DNA cargo independent of DNA repair remains an unsolved challenge of genome editing. Current gene integration approaches require double strand breaks that evoke DNA damage responses and rely on repair pathways that are inactive in terminally differentiated cells. Furthermore, CRISPR-based approaches that bypass double stranded breaks, such as Prime editing, are limited to modification or insertion of short sequences.

There is a need in the art for techniques which address and overcome these shortcomings and enable the co-delivery of gene editor constructs and associated donor templates for the insertion and/or deletion of large sequences into cells for therapeutic and circuit-based uses for broad purposes, across eukaryotic as well as prokaryotic systems.

### 4. SUMMARY

The present disclosure describes co-delivering (i.e., “dual delivery”) to a cell a (i) gene editor construct and a (ii) donor (i.e., “cargo” or “payload”) template that enables in vivo beacon placement and in vivo integration of a template polynucleotide. In typical embodiments, the gene editor construct is comprised of a polynucleotide sequence that encodes the gene editor construct. In typical embodiments, the gene editor construct, upon polynucleotide expression or direct delivery of the gene editor protein and associated guide RNAs (gRNAs (e.g., atgRNA), can incorporate an integrase target recognition site (i.e., “beacon” or “landing pad”) or a recombinase target recognition site at a DNA locus. The gene editor polynucleotide construct is packaged within a lipid nanoparticle (LNP) that is capable of localizing the gene editor polynucleotide construct to a cell cytoplasm. The gene editor polynucleotide construct packaged in a LNP is co-delivered with a donor template (i.e., “cargo” or “payload”) polynucleotide construct packaged into a separate vector that is capable of localizing the donor template to a cell nucleus. In certain embodiments, the donor template vector is AAV, helper dependent adenovirus, or integration deficient lentivirus. In typical embodiments, the donor template is integrated into the genomic integrase target recognition site by an integrase, optionally by an integrase fused/linked to a gene editor protein. Also provided herein are methods using LNP mixtures, including a split LNP approach to deliver precise ratios of mRNA encoding the gene editor protein to atgRNAs. These ratios enable robust in vivo beacon placement in both neonatal and adult mice model systems.

The present disclosure provides a co-delivery platform for site-specific genetic engineering using Programmable Addition via Site-Specific Targeting Elements (PASTE) (see Ionnidi et al.; doi: 10.1101/2021.11.01.466786; the entirety of Ionnidi et al. is incorporated by reference), transposon-mediated gene editing, or other suitable gene editing or gene incorporation technology.

Described herein is a method of co-delivering (i.e., “dual delivery”) to a cell a (i) gene editor construct and a (ii) template polynucleotide (i.e., “cargo” or “payload”). In typical embodiments, the gene editor construct is comprised of a polynucleotide sequence that encodes the gene editor construct. In typical embodiments, the gene editor construct, upon polynucleotide expression or direct delivery of the gene editor protein and associated guide RNAs, can incorporate an integrase target recognition site (i.e., “beacon” or “landing pad”) or a recombinase target recognition site at a DNA locus. The gene editor polynucleotide construct is packaged within a lipid nanoparticle (LNP) that is capable of localizing the gene editor polynucleotide construct to a cell cytoplasm. The gene editor can be packaged into the LNP as a protein along with associated guide RNAs and delivered to the cell cytoplasm or to cell nucleus. The gene editor polynucleotide construct packaged in a LNP is co-delivered with a donor template (i.e., “cargo” or “payload”) polynucleotide construct packaged into a separate vector that is capable of localizing the donor template to a cell nucleus. In certain embodiments, the donor template vector is AAV, helper dependent adenovirus, or integration deficient lentivirus. In typical embodiments, the donor template is integrated into the genomic integrase target recognition site by an integrase, optionally by an integrase fused/linked to a gene editor protein.

The present disclosure provides a co-delivery platform for site-specific genetic engineering using Programmable Addition via Site-Specific Targeting Elements (PASTE) (see Ionnidi et al.; doi: 10.1101/2021.11.01.466786; U.S. application Ser. No. 17/649,308; PCT Publication No. WO 2022/087235A; each of which is herein incorporated by reference in its entirety), transposon-mediated gene editing, or other suitable gene editing or gene incorporation technology.

In one aspect, this disclosure features a method for delivering a system capable of site-specifically integrating a template polynucleotide into the genome of a cell, the method comprising:
delivering to a cell:
   (a) a lipid nanoparticle (LNP) comprising:
      (i) a gene editor polynucleotide; and
   (b) a vector comprising:
      (i) a template polynucleotide, and
      (ii) at least a first attachment site-containing guide RNA (atgRNA).

In some embodiments, the gene editor polynucleotide is capable of localizing to a cell cytoplasm.

In some embodiments, the template polynucleotide is capable of localizing to a cell nucleus.

In some embodiments, the gene editor polynucleotide comprises: a polynucleotide sequence encoding a prime editor system.

In some embodiments, the prime editor system comprises a nucleotide sequence encoding a nickase and a nucleotide sequence encoding a reverse transcriptase.

In some embodiments, the nucleotide sequence encoding the nickase and the nucleotide sequence encoding the reverse transcriptase are positioned in the gene editor polynucleotide such that when expressed the nickase is linked to the reverse transcriptase.

In some embodiments, the nickase is linked to the reverse transcriptase by in-frame fusion. In some embodiments, the nickase is linked to the reverse transcriptase by a linker. In some embodiments, the linker is a peptide fused in-frame between the nickase and reverse transcriptase.

In some embodiments, the gene editor polynucleotide further comprises: a polynucleotide sequence encoding at least a first integrase.

In some embodiments, the linked nickase-reverse transcriptase are further linked to the first integrase.

In some embodiments, the method also includes co-delivering a second vector.

In some embodiments, the second vector comprises a polynucleotide sequence encoding at least a first integrase.

In some embodiments, the first integrase is selected from BxB1, Bcec, Sscd, Sacd, Int10, or Pa01.

In some embodiments, the gene editor polynucleotide further comprises a polynucleotide sequence encoding a recombinase. In some embodiments, the recombinase is FLP or Cre.

In some embodiments, the first atgRNA comprises: (i) a domain that is capable of guiding the prime editor system to a target sequence; and (ii) a reverse transcriptase (RT) template that comprises at least a portion of an at least first integration recognition site.

In some embodiments, the RT template comprises the entirety of the first integration recognition site.

In some embodiments, the vector further comprises a second atgRNA.

In some embodiments, the first atgRNA and the second atgRNA are an at least first pair of atgRNAs, wherein the at least first pair of atgRNAs have domains that are capable of guiding the prime editor system to a target sequence; the first atgRNA further includes a first RT template that comprises at least a portion of an at least first integration recognition site; the second atgRNA further includes a second RT template that comprises at least a portion of the first integration recognition site, and the first atgRNA and the second atgRNAs collectively encode the entirety of the first integration recognition site.

In some embodiments, the vector further comprises a nicking gRNA.

In some embodiments, the LNPs further comprises a nicking gRNA.

In some embodiments, the template polynucleotide comprises at least one of the following: a gene, an expression cassette, a logic gate system, or any combination thereof.

In some embodiments, the template polynucleotide comprises a second integration recognition site.

In some embodiments, the second integration recognition site is a cognate pair with the first integration recognition site.

In some embodiments, the template polynucleotide comprises at least a third integration recognition site.

In some embodiments, the template polynucleotide further comprises at least a fourth integration recognition site.

In some embodiments, the third integration recognition site and the fourth integration recognition site are selected from attB, attB2, attP, or attP2.

In some embodiments, the vector further comprises a sub-sequence that is capable of self-circularizing to form a self-circular nucleic acid.

In some embodiments, the sub-sequence of the vector that is capable of self-circularizing includes the template polynucleotide, whereby upon self-circularizing the self-circular nucleic acid comprises the template polynucleotide.

In some embodiments, the sub-sequence is flanked by the third integration recognition site and the fourth integration recognition site.

In some embodiments, self-circularizing is mediated by recombination of the third integration recognition site and a fourth integration recognition site by the integrase.

In some embodiments, the self-circular nucleic acid comprises one or more additional integration recognition sites that enable integration of additional nucleic acid cargo.

In some embodiments, the vector is a vector selected from: an adenovirus, an AAV, a lentivirus, a HSV, an annelovirus, a retrovirus, a Doggybone™ DNA (dbDNA), a minicircle, a plasmid, a miniDNA, an exosome, a fusosome, or a nanoplasmid.

In some embodiments, the LNP and the vector are concurrently delivered.

In some embodiments, the LNP and the vector are delivered separately.

In some embodiments, the LNP and the vector are delivered at least 1 day, 2 days, 3 days, 4 days, 5 days, 6 days, 7 days, 2 weeks, 3 weeks, 4 weeks, 5 weeks, 6 weeks, 7 weeks, or 8 weeks apart.

In some embodiments, the cell is in vivo.

In another aspect, this disclosure features a method for delivering a system capable of site-specifically integrating a template polynucleotide into the genome of a cell, the method comprising:
delivering to a cell:
   (a) a lipid nanoparticle (LNP) comprising:
      (i) a gene editor polynucleotide, and
      (ii) a first attachment site-containing guide RNA (atgRNA); and
   (b) a vector comprising:
      (i) a template polynucleotide, and
      (ii) a second atgRNA.

In another aspect, this disclosure features a method for delivering a system capable of site-specifically integrating a template polynucleotide into the genome of a cell, the method comprising:
delivering:
   (a) a lipid nanoparticle (LNP) comprising:
      (i) a gene editor polynucleotide,
      (ii) a first attachment site-containing guide RNA (atgRNA), and
      (iii) a second atgRNA; and
   (b) a vector comprising:
      (i) a template polynucleotide.

In another aspect, this disclosure features a method for delivering a system capable of site-specifically integrating a template polynucleotide into the genome of a cell, the method comprising:
delivering:
   (a) a lipid nanoparticle (LNP) comprising:
      (i) a gene editor polynucleotide, and
      (ii) a first attachment site-containing guide RNA (atgRNA); and
   (b) a vector comprising:
      (i) a template polynucleotide, and
      (ii) a nicking atgRNA.

In some embodiments, the gene editor polynucleotide comprises:
a polynucleotide sequence encoding a prime editor system.

In some embodiments, the prime editor system comprises a nucleotide sequence encoding a nickase and a nucleotide sequence encoding a reverse transcriptase.

In some embodiments, the nucleotide sequence encoding the nickase and the nucleotide sequence encoding the reverse transcriptase are positioned in the gene editor polynucleotide such that when expressed the nickase is linked to the reverse transcriptase.

In some embodiments, the nickase is linked to the reverse transcriptase by in-frame fusion.

In some embodiments, the nickase is linked to the reverse transcriptase by a linker.

In some embodiments, the linker is a peptide fused in-frame between the nickase and reverse transcriptase.

In some embodiments, the gene editor polynucleotide construct further comprises: a polynucleotide sequence encoding at least a first integrase.

In some embodiments, the linked nickase-reverse transcriptase are further linked to the integrase.

In some embodiments, the method also includes delivering a second vector.

In some embodiments, the second vector comprises a polynucleotide sequence encoding at least a first integrase.

In some embodiments, the first integrase is selected from BxB1, Bcec, Sscd, Sacd, Int10, or Pa01.

In some embodiments, the gene editor polynucleotide construct further comprises a polynucleotide sequence encoding a recombinase.

In some embodiments, the recombinase is FLP or Cre.

In some embodiments, the first atgRNA comprises: (i) a domain that is capable of guiding the prime editor system to a target sequence; and (ii) a reverse transcriptase (RT) template that comprises at least a portion of an at least first integration recognition site.

In some embodiments, the RT template comprises the entirety of the first integration recognition site.

In some embodiments, the first atgRNA and the second atgRNA are an at least first pair of atgRNAs, wherein the at least first pair of atgRNAs have domains that are capable of guiding the prime editor system to a target sequence; the first atgRNA further includes a first RT template that comprises at least a portion of the first integration recognition site; the second atgRNA further includes a second RT template that comprises at least a portion of the first integration recognition site, and the first atgRNA and the second atgRNAs collectively encode the entirety of the first integration recognition site.

In some embodiments, the template polynucleotide comprises at least one of the following: a gene, an expression cassette, a logic gate system, or any combination thereof.

In some embodiments, the template polynucleotide comprises a second integration recognition site.

In some embodiments, the second integration recognition site is a cognate pair with the first integration recognition site.

In some embodiments, the template polynucleotide comprises at least a third integration recognition site.

In some embodiments, the template polynucleotide further comprises at least a fourth integration recognition site.

In some embodiments, the third integration recognition site and the fourth integration recognition site are selected from attB, attB2, attP, or attP2.

In some embodiments, the vector further comprises a sub-sequence that is capable of self-circularizing to form a self-circular nucleic acid.

In some embodiments, the sub-sequence of the vector that is capable of self-circularizing includes the template polynucleotide, whereby upon self-circularizing the self-circular nucleic acid comprises the template polynucleotide.

In some embodiments, the sub-sequence is flanked by the third integration recognition site and the fourth integration recognition site.

In some embodiments, self-circularizing is mediated by recombination of the third integration recognition site and the fourth integration recognition site by the integrase.

In some embodiments, the self-circular nucleic acid comprises one or more additional integration recognition sites that enable integration of additional nucleic acid cargo.

In some embodiments, the vector is a vector selected from: an adenovirus, an AAV, a lentivirus, a HSV, an annelovirus, a retrovirus, a Doggybone™ DNA (dbDNA), a minicircle, a plasmid, a miniDNA, a exosome, a fusosome, or a nanoplasmid.

In some embodiments, the LNP and the vector are concurrently delivered.

In some embodiments, the LNP and the vector are delivered separately.

In some embodiments, the LNP and the vector are delivered at least 1 day, 2 days, 3 days, 4 days, 5 days, 6 days, 7 days, 2 weeks, 3 weeks, 4 weeks, 5 weeks, 6 weeks, 7 weeks, or 8 weeks apart.

In some embodiments, the cell is in vivo.

In another aspect, this disclosure features a method of co-delivering a system capable of site-specifically integrating at least a first integration recognition site into the genome of a cell, the method comprising:
co-delivering to a cell:
   (a) a first lipid nanoparticle (LNP) comprising:
      (i) a first gene editor polynucleotide, and
      (ii) a first attachment site-containing guide RNA (atgRNA); and
   (b) a second lipid nanoparticle (LNP) comprising:
      (i) a second gene editor polynucleotide, and
      (ii) a second attachment site-containing guide RNA (atgRNA),
   wherein the first atgRNA and the second atgRNA are an at least first pair of atgRNAs.

In some embodiments, the method also includes mixing the first LNP and the second LNP prior to co-delivering to the cell.

In some embodiments, the first LNP and the second LNP are mixed at a ratio of 1:0.25, 1:0.5, 1:0.75, 1:1, 0.75:1, 0.5:1, or 0.25:1.

In some embodiments, the first gene editor polynucleotide construct, the second gene editor polynucleotide construct, or both comprise: a polynucleotide sequence encoding a prime editor system.

In some embodiments, the prime editor system comprises a nucleotide sequence encoding a nickase and a nucleotide sequence encoding a reverse transcriptase.

In some embodiments, the nucleotide sequence encoding the nickase and the nucleotide sequence encoding the reverse transcriptase are positioned in the gene editor polynucleotide such that when expressed the nickase is linked to the reverse transcriptase.

In some embodiments, the nickase is linked to the reverse transcriptase by in-frame fusion.

In some embodiments, the nickase is linked to the reverse transcriptase by a linker.

In some embodiments, the linker is a peptide fused in-frame between the nickase and reverse transcriptase.

In some embodiments, the first gene editor polynucleotide, construct, the second gene editor polynucleotide construct, or both, further comprise:
a polynucleotide sequence encoding an integrase.

In some embodiments, the linked nickase-reverse transcriptase are further linked to the integrase.

In some embodiments, the first gene editor polynucleotide, the second gene editor polynucleotide, or both, further comprise: a polynucleotide sequence encoding a recombinase.

In some embodiments, the linked nickase-reverse transcriptase are further linked to the recombinase.

In some embodiments, the first gene editor polynucleotide and the second gene editor polynucleotide are the same.

In some embodiments, the first gene editor polynucleotide is mRNA, the second gene editor polynucleotide is mRNA, or both the first and second gene editor polynucleotides are mRNA.

In some embodiments, the first LNP comprises a ratio of mRNA to atgRNA of 1:0.25, 1:0.5, 1:0.75, 1:1, 0.75:1, 0.5:1, or 0.25:1.

In some embodiments, the second LNP comprises a ratio of mRNA to atgRNA of 1:0.25, 1:0.5, 1:0.75, 1:1, 0.75:1, 0.5:1, or 0.25:1.

In some embodiments, the method also includes delivering an integrase.

In some embodiments, delivering the integrase comprises co-delivering the integrase with (a) and (b).

In some embodiments, the method comprises delivering a polynucleotide sequence encoding the integrase.

In some embodiments, the polynucleotide sequence is encoded in a first vector.

In some embodiments, the first vector is a vector selected from: an adenovirus, an AAV, a lentivirus, a HSV, an annelovirus, a retrovirus, a Doggybone™ DNA (dbDNA), a minicircle, a plasmid, a miniDNA, an exosome, a fusosome, or a nanoplasmid.

In some embodiments, the first vector further comprises a template polynucleotide and a sequence that is an integration cognate with the first integration recognition site.

In some embodiments, the method also includes delivering a recombinase.

In some embodiments, delivering the recombinase comprises co-delivering the recombinase with (a) and (b).

In some embodiments, the method comprises delivering a polynucleotide sequence encoding the recombinase.

In some embodiments, the polynucleotide sequence is encoded in the first vector.

In some embodiments, the method also includes delivering a second vector.

In some embodiments, the second vector comprises a template polynucleotide and a sequence that is an integration cognate with the first integration recognition site.

In some embodiments, the second vector is a vector selected from: an adenovirus, an AAV, a lentivirus, an HSV, an annelovirus, a retrovirus, Doggybone™ DNA (dbDNA), a minicircle, a plasmid, a miniDNA, an exosome, a fusosome, or a nanoplasmid.

In some embodiments, the template polynucleotide comprises at least one of the following: a gene, an expression cassette, a logic gate system, or any combination thereof.

In some embodiments, the template polynucleotide comprises a second integration recognition site.

In some embodiments, the second integration recognition site is a cognate pair with the first integration recognition site.

In some embodiments, the template polynucleotide comprises at least a third integration recognition site.

In some embodiments, the template polynucleotide further comprises at least a fourth integration recognition site.

In some embodiments, the third integration recognition site and the fourth integration recognition site are selected from attB, attB2, attP, or attP2.

In some embodiments, the vector further comprises a sub-sequence that is capable of self-circularizing to form a self-circular nucleic acid.

In some embodiments, the sub-sequence of the vector that is capable of self-circularizing includes the template polynucleotide, whereby upon self-circularizing the self-circular nucleic acid comprises the template polynucleotide.

In some embodiments, the sub-sequence is flanked by the third integration recognition site and the fourth integration recognition site.

In some embodiments, self-circularizing is mediated by recombination of the third integration recognition site and a fourth integration recognition site by the integrase.

In some embodiments, the self-circular nucleic acid comprises one or more additional integration recognition sites that enable integration of additional nucleic acid cargo.

In some embodiments, the at least first pair of atgRNAs have domains that are capable of guiding the prime editor system to a target sequence; the first atgRNA further includes a first RT template that comprises at least a portion of a first integration recognition site; the second atgRNA further includes a second RT template that comprises at least a portion of the first integration recognition site, and the first atgRNA and the second atgRNAs collectively encode the entirety of the first integration recognition site.

In some embodiments, the first integration site is an AttB sequence, a FRT sequence, or a VOX sequence.

In some embodiments, the first atgRNA, the second atgRNA or both are synthetic.

In some embodiments, the integrase is selected from BxB1, Bcec, Sscd, Sacd, Int10, or Pa01.

In some embodiments, the cell is in vivo.

In another aspect, this disclosure features a system capable of site-specifically integrating a template polynucleotide into the genome of a cell, the system comprising:
(a) a lipid nanoparticle (LNP) comprising:
   (i) a gene editor polynucleotide construct; and
(b) a vector comprising:
   (i) a template polynucleotide, and
   (ii) at least a first attachment site-containing guide RNA (atgRNA).

In some embodiments of the system, the gene editor polynucleotide construct comprises a polynucleotide sequence encoding a prime editor system.

In some embodiments of the system, the prime editor system comprises a nucleotide sequence encoding a nickase and a nucleotide sequence encoding a reverse transcriptase.

In some embodiments of the system, the nucleotide sequence encoding the nickase and the nucleotide sequence encoding the reverse transcriptase are positioned in the gene editor polynucleotide such that when expressed the nickase is linked to the reverse transcriptase. In some embodiments of the system, the nickase is linked to the reverse transcriptase by in-frame fusion. In some embodiments of the system, the nickase is linked to the reverse transcriptase by a linker.

In some embodiments of the system, the linker is a peptide fused in-frame between the nickase and reverse transcriptase.

In some embodiments of the system, the gene editor polynucleotide construct further comprises: a polynucleotide sequence encoding at least a first integrase.

In some embodiments of the system, the linked nickase-reverse transcriptase are further linked to the first integrase.

In some embodiments of the system, the system also includes a second vector.

In some embodiments of the system, the second vector comprises a polynucleotide sequence encoding at least a first integrase. In some embodiments of the system, the first integrase is selected from BxB1, Bcec, Sscd, Sacd, Int10, or Pa01.

In some embodiments of the system, the gene editor polynucleotide construct further comprises a polynucleotide sequence encoding a recombinase. In some embodiments of the system, the recombinase is FLP or Cre.

In some embodiments of the system, the first atgRNA comprises: (i) a domain that is capable of guiding the prime editor system to a target sequence; and (ii) a reverse transcriptase (RT) template that comprises at least a portion of an at least first integration recognition site.

In some embodiments of the system, the RT template comprises the entirety of the first integration recognition site.

In some embodiments of the system, the vector further comprises a second atgRNA.

In some embodiments of the system, the first atgRNA and the second atgRNA are an at least first pair of atgRNAs, wherein the at least first pair of atgRNAs have domains that are capable of guiding the prime editor system to a target sequence; the first atgRNA further includes a first RT template that comprises at least a portion of the first integration recognition site; the second atgRNA further includes a second RT template that comprises at least a portion of the first integration recognition site, and the first atgRNA and the second atgRNAs collectively encode the entirety of the first integration recognition site.

In some embodiments of the system, the vector further comprises a nicking gRNA.

In some embodiments of the system, the LNP further comprises a nicking gRNA.

In some embodiments of the system, the template polynucleotide comprises at least one of the following: a gene, an expression cassette, a logic gate system, or any combination thereof.

In some embodiments of the system, the template polynucleotide comprises a second integration recognition site.

In some embodiments of the system, the second integration recognition site is a cognate pair with the first integration recognition site.

In some embodiments of the system, the template polynucleotide comprises at least a third integration recognition site.

In some embodiments of the system, the template polynucleotide construct further comprises at least a fourth integration recognition site.

In some embodiments of the system, the third integration recognition site and the fourth integration recognition site are selected from attB, attB2, attP, or attP2.

In some embodiments of the system, the vector further comprises a sub-sequence that is capable of self-circularizing to form a self-circular nucleic acid.

In some embodiments of the system, the sub-sequence of vector that is capable of self-circularizing includes the template polynucleotide, whereby upon self-circularizing the self-circular nucleic acid comprises the template polynucleotide.

In some embodiments of the system, the sub-sequence is flanked by the third integration recognition site and the fourth integration recognition site.

In some embodiments of the system, self-circularizing is mediated by recombination of the third integration recognition site and the fourth integration recognition site by the integrase.

In some embodiments of the system, the self-circular nucleic acid comprises one or more additional integration recognition sites that enable integration of additional nucleic acid cargo.

In some embodiments of the system, the vector is a recombinant adenovirus, a helper dependent adenovirus, or an adeno-associated virus.

In another aspect, this disclosure features a system capable of site-specifically integrating a template polynucleotide into the genome of a cell, the system comprising:
(a) a lipid nanoparticle (LNP) comprising:
   (i) a gene editor polynucleotide, and
   (ii) a first attachment site-containing guide RNA (atgRNA); and
(b) a vector comprising:
   (i) a template polynucleotide, and
   (ii) a second atgRNA.

In another aspect, this disclosure features a system capable of site-specifically integrating a template polynucleotide into the genome of a cell, the system comprising:
(a) a lipid nanoparticle (LNP) comprising
   (i) a gene editor polynucleotide,
   (ii) a first attachment site-containing guide RNA (atgRNA), and
   (iii) a second atgRNA; and
(b) a vector comprising:
   (i) a template polynucleotide.

In another aspect, this disclosure features a system capable of site-specifically integrating a template polynucleotide into the genome of a cell, the system comprising:
(a) a lipid nanoparticle (LNP) comprising
   (i) a gene editor polynucleotide, and
   (ii) a first attachment site-containing guide RNA (atgRNA); and
(b) a vector comprising:
   (i) a template polynucleotide, and
   (ii) a nicking gRNA.

In some embodiments of the system, the gene editor polynucleotide comprises: a polynucleotide sequence encoding a prime editor system.

In some embodiments of the system, the prime editor system comprises a nucleotide sequence encoding a nickase and a nucleotide sequence encoding a reverse transcriptase.

In some embodiments of the system, the nucleotide sequence encoding the nickase and the nucleotide sequence encoding the reverse transcriptase are positioned in the gene editor polynucleotide such that when expressed the nickase is linked to the reverse transcriptase. In some embodiments of the system, the nickase is linked to the reverse transcriptase by in-frame fusion. In some embodiments of the system, the nickase is linked to the reverse transcriptase by a linker.

In some embodiments of the system, the linker is a peptide fused in-frame between the nickase and reverse transcriptase.

In some embodiments of the system, the gene editor polynucleotide further comprises:
a polynucleotide sequence encoding at least a first integrase.

In some embodiments of the system, the linked nickase-reverse transcriptase are further linked to the first integrase.

In some embodiments of the system, the system also includes a second vector.

In some embodiments of the system, the second vector comprises a polynucleotide sequence encoding at least a first integrase.

In some embodiments of the system, the first integrase is selected from BxB1, Bcec, Sscd, Sacd, Int10, or Pa01.

In some embodiments of the system, the gene editor polynucleotide further comprises a polynucleotide sequence encoding a recombinase.

In some embodiments of the system, the recombinase is FLP or Cre.

In some embodiments of the system, the first atgRNA comprises: (i) a domain that is capable of guiding the prime editor system to a target sequence; and (ii) a reverse transcriptase (RT) template that comprises at least a portion of an at least first integration recognition site.

In some embodiments of the system, the RT template comprises the entirety of the first integration recognition site.

In some embodiments of the system, the first atgRNA and the second atgRNA are an at least first pair of atgRNAs, wherein the at least first pair of atgRNAs have domains that are capable of guiding the prime editor system to a target sequence; the first atgRNA further includes a first RT template that comprises at least a portion of the first integration recognition site; the second atgRNA further includes a second RT template that comprises at least a portion of the first integration recognition site, and the first atgRNA and the second atgRNAs collectively encode the entirety of the first integration recognition site.

In some embodiments of the system, the template polynucleotide comprises at least one of the following: a gene, an expression cassette, a logic gate system, or any combination thereof.

In some embodiments of the system, the template polynucleotide comprises a second integration recognition site.

In some embodiments of the system, the second integration recognition site is a cognate pair with the first integration recognition site.

In some embodiments of the system, the template polynucleotide comprises at least a third integration recognition site.

In some embodiments of the system, the template polynucleotide construct further comprises at least a fourth integration recognition site.

In some embodiments of the system, the third integration recognition site and the fourth integration recognition site are selected from attB, attB2, attP, or attP2.

In some embodiments of the system, the vector further comprises a sub-sequence that is capable of self-circularizing to form a self-circular nucleic acid.

In some embodiments of the system, the sub-sequence of vector that is capable of self-circularizing includes the template polynucleotide, whereby upon self-circularizing the self-circular nucleic acid comprises the template polynucleotide.

In some embodiments of the system, the sub-sequence is flanked by the third integration recognition site and the fourth integration recognition site.

In some embodiments of the system, self-circularizing is mediated by recombination of the third integration recognition site and the fourth integration recognition site by the integrase.

In some embodiments of the system, the self-circular nucleic acid comprises one or more additional integration recognition sites that enable integration of additional nucleic acid cargo.

In some embodiments of the system, the vector is recombinant adenovirus, helper dependent adenovirus, or an adeno-associated virus.

In another aspect, this disclosure features a system capable of site-specifically integrating at least a first integration recognition site into the genome of a cell, the system comprising:
(a) a first lipid nanoparticle (LNP) comprising:
   (i) a first gene editor polynucleotide, and
   (ii) a first attachment site-containing guide RNA (atgRNA); and
(b) a second lipid nanoparticle (LNP) comprising:
   (i) a second gene editor polynucleotide, and
   (ii) a second attachment site-containing guide RNA (atgRNA).

In some embodiments of the system, the first atgRNA and the second atgRNA are an at least first pair of atgRNAs.

In some embodiments of the system, the first LNP and the second LNP are mixed at a ratio of 1:0.25, 1:0.5, 1:0.75, 1:1, 0.75:1, 0.5:1, or 0.25:1.

In some embodiments of the system, the first gene editor polynucleotide, the second gene editor polynucleotide, or both comprise:
a polynucleotide sequence encoding a prime editor system.

In some embodiments of the system, the prime editor system comprises a nucleotide sequence encoding a nickase and a nucleotide sequence encoding a reverse transcriptase.

In some embodiments of the system, the nucleotide sequence encoding the nickase and the nucleotide sequence encoding the reverse transcriptase are positioned in the gene editor polynucleotide such that when expressed the nickase is linked to the reverse transcriptase.

In some embodiments of the system, the nickase is linked to the reverse transcriptase by in-frame fusion.

In some embodiments of the system, the nickase is linked to the reverse transcriptase by a linker.

In some embodiments of the system, the linker is a peptide fused in-frame between the nickase and reverse transcriptase.

In some embodiments of the system, the first gene editor polynucleotide, the second gene editor polynucleotide, or both, further comprise: a polynucleotide sequence encoding an integrase.

In some embodiments of the system, the linked nickase-reverse transcriptase are further linked to the integrase.

In some embodiments of the system, the first gene editor polynucleotide, the second gene editor polynucleotide, or both, further comprise: a polynucleotide sequence encoding a recombinase.

In some embodiments of the system, the nickase-reverse transcriptase are further linked to the recombinase.

In some embodiments of the system, the first gene editor polynucleotide and the second gene editor polynucleotide are the same.

In some embodiments of the system, the first gene editor polynucleotide is mRNA, the second gene editor polynucleotide is mRNA, or both the first and second gene editor polynucleotides are mRNA.

In some embodiments of the system, the first LNP comprises a ratio of mRNA to atgRNA of 1:0.25, 1:0.5, 1:0.75, 1:1, 0.75:1, 0.5:1, or 0.25:1.

In some embodiments of the system, the second LNP comprises a ratio of mRNA to atgRNA of 1:0.25, 1:0.5, 1:0.75, 1:1, 0.75:1, 0.5:1, or 0.25:1.

In some embodiments of the system, the system also includes an integrase.

In some embodiments of the system, the system comprises a polynucleotide sequence encoding the integrase.

In some embodiments of the system, the polynucleotide sequence is encoded in a first vector.

In some embodiments of the system, the first vector is a vector selected from: an adenovirus, an AAV, a lentivirus, a HSV, an annelovirus, a retrovirus, a Doggybone™ DNA (dbDNA), a minicircle, a plasmid, a miniDNA, an exosome, a fusosome, or a nanoplasmid.

In some embodiments of the system, the first vector further comprises a template polynucleotide and a sequence that is an integration cognate with the first integration recognition site.

In some embodiments of the system, the system also includes delivering a recombinase.

In some embodiments of the system, delivering the recombinase comprises co-delivering the recombinase with (a) and (b).

In some embodiments of the system, the system comprises delivering a polynucleotide sequence encoding the recombinase.

In some embodiments of the system, the polynucleotide sequence is encoded in the first vector.

In some embodiments of the system, the system also includes co-delivering a second vector.

In some embodiments of the system, the second vector comprises a template polynucleotide and a sequence that is an integration cognate with the first integration recognition site.

In some embodiments of the system, the second vector is a vector selected from: an adenovirus, an AAV, a lentivirus, a HSV, an annelovirus, a retrovirus, a Doggybone™ DNA (dbDNA), a minicircle, a plasmid, a miniDNA, an exosome, a fusosome, or a nanoplasmid.

In some embodiments of the system, the template polynucleotide comprises at least one of the following: a gene, an expression cassette, a logic gate system, or any combination thereof.

In some embodiments of the system, the template polynucleotide comprises a second integration recognition site.

In some embodiments of the system, the second integration recognition site is a cognate pair with the first integration recognition site.

In some embodiments of the system, the template polynucleotide comprises at least a third integration recognition site.

In some embodiments of the system, the template polynucleotide further comprises at least a fourth integration recognition site.

In some embodiments of the system, the third integration recognition site and the fourth integration recognition site are selected from attB, attB2, attP, or attP2.

In some embodiments of the system, the vector further comprises a sub-sequence that is capable of self-circularizing to form a self-circular nucleic acid.

In some embodiments of the system, the sub-sequence of the vector that is capable of self-circularizing includes the template polynucleotide, whereby upon self-circularizing the self-circular nucleic acid comprises the template polynucleotide.

In some embodiments of the system, the sub-sequence is flanked by the third integration recognition site and the fourth integration recognition site.

In some embodiments of the system, self-circularizing is mediated by recombination of the third integration recognition site and the fourth integration recognition site by the integrase.

In some embodiments of the system, the self-circular nucleic acid comprises one or more additional integration recognition sites that enable integration of additional nucleic acid cargo.

In some embodiments of the system, the at least first pair of atgRNAs have domains that are capable of guiding the prime editor system to a target sequence; the first atgRNA further includes a first RT template that comprises at least a portion of a first integration recognition site; the second atgRNA further includes a second RT template that comprises at least a portion of the first integration recognition site, and the first atgRNA and the second atgRNAs collectively encode the entirety of the first integration recognition site.

In some embodiments, the first integration site is an AttB sequence, a FRT sequence, or a VOX sequence.

In some embodiments of the system, the first atgRNA, the second atgRNA or both are synthetic.

In some embodiments of the system, the integrase is selected from BxB1, Bcec, Sscd, Sacd, Int10, or Pa01.

In some embodiments of the system, the recombinase is FLP or Cre.

In another aspect, this disclosure features a cell comprising any of the delivery systems or any of the co-delivery systems described herein.

In another aspect, this disclosure features a pharmaceutical composition comprising the any of the delivery systems described herein or any of the co-delivery systems described herein.

In another aspect, this disclosure features a method of treating a patient in need thereof, the method comprising administering an effective amount of any of the systems described herein, any of the cells described herein, or any of the pharmaceutical compositions described herein.

In another aspect, this disclosure features a method of treating a patient in need thereof, the method comprising: administering an effective amount of any of the LNPs described herein, any of the first vectors described herein, or any of the second vectors described herein as a first dose and an effective amount of any of the LNPs described herein, any of the first vectors described herein, or any of the second vectors described herein as a second dose.

In some embodiments, the first dose and the second dose are separately administered by multiple administrations.

In some embodiments, the first dose and the second dose are administered at least 1 day, 2 days, 3 days, 4 days, 5 days, 6 days, or 7 days apart.

In some embodiments, the first dose and the second dose are administered at least 2 weeks, 3 weeks, 4 weeks, 5 weeks, 6 weeks, 7 weeks, or 8 weeks apart.

### 5. BRIEF DESCRIPTION OF THE DRAWINGS

These and other features, aspects, and advantages of the present invention will become better understood with regard to the following description, and accompanying drawings, where:

FIG. 1 shows a non-limiting illustration of a gene editor construct packaged within a lipid nanoparticle (LNP).

FIG. 2 illustrates the donor template (i.e., “cargo” or “payload” or “template polynucleotide”)) packaged within a vector.

FIG. 3 illustrates integrase-mediated self-circularization of the donor template (template polynucleotide) within viral genome. The circularized donor template is capable of being genomically incorporated into an orthogonal integrase target recognition site (i.e., “beacon”).

FIG. 4 shows non-limiting illustrations of a gene editor construct packaged within a lipid nanoparticle and an atgRNA, ngRNA, and donor template (i.e., template polynucleotide encoding a gene of interest) packaged within a vector. GOI=gene of interest. PGI=programmable gene insertion. U6=U6 promoter. atgRNA=attachment site-containing guide RNA (atgRNA).

FIG. 5 shows non-limiting illustrations of a gene editor construct (e.g., mRNA encoding PE2-BxB1) and a nicking guide RNA (ngRNA) packaged within a lipid nanoparticle (LNP) and an atgRNA and donor template (i.e., template polynucleotide encoding a gene of interest) packaged within a vector.

FIGS. 6A-6B show non-limiting illustrations of three self-complementary AAV (scAAV) genomes capable of recombinase/integrase-mediated self-circularization. FIG. 6A shows the structure of the three self-complementary AAV (scAAV) genomes capable of recombinase/integrase-mediated self-circularization. FIG. 6B shows non-limiting examples of sequences that enable self-circularization (e.g., LoxP AttP GT (SEQ ID NO: 568 and SEQ ID NO: 569); FRT AttP GT (SEQ ID NO: 570 and SEQ ID NO: 571); and AttB CC AttP GT (SEQ ID NO: 572 and SEQ ID NO: 573)). GT indicates an AttP site with a GT dinucleotide. AttB CC indicates an AttB site with a CC dinucleotide. LoxP=a LoxP recombinase recognition site. FRT=a FRT recombinase recognition site.

FIG. 7 shows a non-limiting illustration of recombinase/integrase-mediated intramolecular circularization products.

FIGS. 8A-8B show non-limiting illustrations of a ddPCR assay and intramolecular circularization ddPCR detection probes. FIG. 8A shows a non-limiting illustration of the ddPCR strategy. FIG. 8B shows non-limiting examples of the universal probe (SEQ ID NO: 574 and SEQ ID NO: 575) and an AttR probe (SEQ ID NO: 576 and SEQ ID NO: 577) that can be used in the assay shown in FIG. 8A.

FIG. 9 shows a non-limiting illustration of a pDNA genome and AAV transfection and screening protocol.

FIG. 10 shows data for circularization of AAV pDNA and packaged AAV genomic DNA with Bxb1.

FIG. 11 shows data for Cre-, FLPe-, and Bxb1-mediated circularization of AAV pDNA confirmed by ddPCR.

FIG. 12 shows Cre-, FLPe-, and Bxb1-mediated circularization of packaged AAV confirmed by ddPCR

FIG. 13 shows percent circularization between the Bxb1-mediated attR scar ddPCR probe (“attR probe” described in FIG. 8B) and the universal ddPCR probe (“universal probe” described in FIG. 8B).

FIGS. 14A-14E shows analysis of AttP variants. FIG. 14A shows a non-limiting schematic of AttP mutations tested for improving integration efficiency (SEQ ID NOS: 394 and 540-542, respectively, in order of appearance). FIG. 14B shows integration efficiencies of wildtype and mutant AttP sites across a panel of AttB lengths. FIG. 14C shows a non-limiting schematic of multiplexed integration of different cargo sets at specific genomic loci. Three fluorescent cargos (GFP, mCherry, and YFP) are inserted orthogonally at three different loci (ACTB, LMNB1, NOLC1) for in-frame gene tagging. FIG. 14D shows orthogonality of top 4 AttB/AttP dinucleotide pairs evaluated for GFP integration with PASTE at the ACTB locus. FIG. 14E shows efficiency of multiplexed PASTE insertion of combinations of fluorophores at ACTB, LMNB1, and NOLC1 loci. Data are mean (n=3) s.e.m.

FIG. 15 illustrates a schematic of single atgRNA and dual atgRNA approaches for beacon placement (“integration recognition site”).

FIG. 16 shows percent beacon placement in primary mouse hepatocytes (PMH) following delivery of mRNA to deliver a polynucleotide encoding a gene editor polynucleotide construct and an AAV to deliver the first and second atgRNA according to the following conditions: (i) concurrent delivery (“co-dose”), (ii) AAV delivery followed by a “1-day delay” before delivery of the mRNA, or (iii) AAV delivery followed by a “2-day delay” before delivery of the mRNA.

FIG. 17 shows percent beacon placement in primary human hepatocytes (PHH) following delivering of mRNA to deliver a polynucleotide encoding a gene editor polynucleotide construct and an AAV to deliver the first and second atgRNA. The mRNA and AAV were delivered concurrently.

FIG. 18 shows percent in vivo beacon placement in the Nolc1 locus of mice following delivery of a polynucleotide encoding a gene editor polynucleotide construct using a lipid nanoparticle (LNP) and a first atgRNA and second atgRNA using an AAV. % BP=% beacon placement. LNP were administered at doses of 0.5 mg/kg, 1.5 mg/kg, 3 mg/kg, and 5 mg/kg. AAV was administered at 1E11, 3E11, or 1E12 viral genomes (vg) per animal. LNP #F1=LNP formulation #1. LNP #F2=LNP formulation #F2. LNP #F3=LNP formulation #F3.

FIG. 19 show percent in vivo integration of a template polynucleotide in AttP mice following delivering of the Bxb1 using adenovirus (AdV) and the template polynucleotide using an AAV (“AAV Cargo”). Bxb1 Adv was administered to the mice at a dose of either 3E10 or 1E11 vector genomes (vg) per animal. AAV Cargo was administered to the mice at a dose of 1E12.

FIG. 20A shows ddPCR data for percent in vivo beacon placement in the Nolc1 locus of neonatal mice at eight days post-delivery of a single dose of a mixture of two LNPs. First LNP contained mRNA encoding a prime editing system and a first synthetic atgRNA (atgRNA1) at a 1:1 ratio. Second LNP contained mRNA encoding a prime editing system and a second synthetic atgRNA (atgRNA2) at a 1:1 ratio. Each of the first and second atgRNAs targeted the mouse Nolc1 locus, encoded a portion of an integration recognition site (“beacon”), and together included a 6 bp overlap. The first and second LNPs were combined 1:1 as mixture and administered at either 1 mg/kg or 3 mg/kg. LNP #F2=LNP formulation #F2.

FIG. 20B show NGS data for percent in vivo beacon placement in the Nolc1 locus of the same neonatal mice and treatment conditions as described in FIG. 20A. NGS data shows beacon placement eight days after administration of the LNP mixture. LNP #F2=LNP formulation #F2.

FIG. 20C shows NGS data for percentage of in vivo beacons placed in the Nolc1 NGS data is for the same mice with the same treatment conditions as described in FIG. 20A. NGS data shows data for eight days after administration of the LNP mixture. LNP #F2=LNP formulation #F2.

FIG. 21A shows ddPCR data for percent in vivo beacon placement in the Nolc1 locus of neonatal mice at 6 weeks post-delivery of a single dose of a mixture of two LNPs. First LNP contained mRNA encoding a prime editing system and a first synthetic atgRNA (atgRNA1) at a 1:1 ratio. Second LNP contained mRNA encoding a prime editing system and a second synthetic atgRNA (atgRNA2) at a 1:1 ratio. Each of the first and second atgRNAs targeted the mouse Nolc1 locus, encoded a portion of an integration recognition site (“beacon”), and together included a 6 bp overlap. The first and second LNPs were combined 1:1 as mixture and administered at either 1 mg/kg or 3 mg/kg. LNP #F2=LNP formulation #F2.

FIG. 21B shows NGS data for percent in vivo beacon placement in the Nolc1 locus of the same neonatal mice and treatment conditions as described in FIG. 21A. NGS data shows beacon placement 6 weeks after administration of the LNP mixture. LNP #F2=LNP formulation #F2.

FIG. 21C shows NGS data for percentage of in vivo beacons placed in the Nolc1 locus that included the expected integration recognition site. Data is from the same mice with the same treatment conditions as described in FIG. 22A. NGS data shows data at 6 weeks after administration of the LNP mixture. LNP #F2=LNP formulation #F2.

FIG. 22A shows ddPCR data for percent in vivo beacon placement in the Factor IX (“mF9”) locus of 6-8 week old mice at day 8 post-delivery of a single dose of a mixture of two LNPs. First LNP contained mRNA encoding a prime editing system and a first synthetic atgRNA (atgRNA1) at a ratio of 1:0.5, 1:1, or 1:2. Second LNP contained mRNA encoding a prime editing system and a second synthetic atgRNA (atgRNA2) at a ratio of 1:1, 1:0.5, or 1:2. Each of the first and second atgRNAs targeted the mouse Factor IX locus, encoded a portion of an integration recognition site (“beacon”), and together included a 6 bp overlap. The first and second LNPs were combined 1:1 as mixture with the final ratio of mRNA:atgRNA1:atgRNA2 at 1:0.25:0.25; 1:0.5:0.5, or 1:1:1. LNP #F2=LNP formulation #F2.

FIG. 22B shows NGS data for percent in vivo beacon placement in the mF9 locus of the same neonatal mice and treatment conditions as described in FIG. 22A. NGS data shows beacon placement 8 days after administration of the LNP mixture. LNP #F2=LNP formulation #F2.

FIG. 22C shows NGS data for percent of in vivo beacons placed in the mF9 locus that included the expected integration recognition site. Data is from the same mice with the same treatment conditions as described in FIG. 22A. NGS data shows data at 8 days after administration of the LNP mixture. LNP #F2=LNP formulation #F2.

### 6. DETAILED DESCRIPTION

Described herein is a method of co-delivering (i.e., “dual delivery”) to a cell a (i) gene editor construct and a (ii) donor (i.e., “cargo” or “payload”) template. The gene editor construct is comprised of a polynucleotide sequence that encodes the gene editor construct. In typical embodiments, the gene editor construct, upon polynucleotide expression or direct delivery of the gene editor protein and associated guide RNAs, can incorporate an integrase target recognition site (i.e., “beacon” or “landing pad”) or a recombinase target recognition site at a DNA locus. The gene editor polynucleotide construct is packaged within a lipid nanoparticle (LNP) that is capable of localizing the gene editor polynucleotide construct to a cell cytoplasm. The gene editor polynucleotide construct packaged in a LNP is co-delivered with a donor template (i.e., “cargo” or “payload”) polynucleotide construct packaged into a separate vector that is capable of localizing the donor template to a cell nucleus. In certain embodiments, the donor template vector is AAV, helper dependent adenovirus, or integration deficient lentivirus. In typical embodiments, the donor template is integrated into the genomic integrase target recognition site by an integrase, optionally by an integrase fused/linked to a gene editor protein.

### 6.1. Terminology

Unless defined otherwise, all technical and scientific terms used herein have the meaning commonly understood by a person skilled in the art to which this invention belongs. As used herein, the following terms have the meanings ascribed to them below.

“Gene editor” as used herein, is a protein that that can be used to perform gene editing, gene modification, gene insertion, gene deletion, or gene inversion. As used herein, the terms “gene editor polynucleotide” refers to polynucleotide sequence encoding the gene editor protein. Such an enzyme or enzyme fusion may contain DNA or RNA targetable nuclease protein (i.e., Cas protein, ADAR, or ADAT), wherein target specificity is mediated by a complexed nucleic acid (i.e., guide RNA). Such an enzyme or enzyme fusion may be a DNA/RNA targetable protein, wherein target specificity is mediated by internal, conjugated, fused, or linked amino acids, such as within TALENs, ZFNs, or meganucleases. The skilled person in the art would appreciate that the gene editor can demonstrate targeted nuclease activity, targeted binding with no nuclease activity, or targeted nickase activity (or cleavase activity). A gene editor comprising a targetable protein may be fused, linked, complexed, operate in cis or trans to one or more proteins or protein fragment motifs. Gene editors may be fused or linked to one or more integrase, recombinase, polymerase, telomerase, reverse transcriptase, or invertase. A gene editor can be a prime editor fusion protein or a gene writer fusion protein.

“Prime editor fusion protein” as used herein, describes a protein that is used in prime editing. “Prime editor system” as used herein describes the components used in prime editing. Prime editing uses CRISPR enzyme that nicks or cuts only single strand of double stranded DNA, i.e., a nickase; the nickase can occur either naturally or by mutation or modification of a nuclease that makes double stranded cuts. The nickase is programmed (directed) with a prime-editing guide RNA (pegRNA). The skilled person in the art would appreciate that the pegRNA both specifies the target site and encodes the desired edit. Described herein are attachment site containing guide RNA (atgRNA) that both specifies the target and encodes for the desired integrase target recognition site. The nickase may be programmed (directed) with an atgRNA. Advantageously the nickase is a catalytically impaired Cas9 endonuclease, a Cas9 nickase, that is fused to the reverse transcriptase. During genetic editing, the Cas9 nickase part of the protein is guided to the DNA target site by the atgRNA (or pegRNA), whereby a nick or single stranded cut occurs. The reverse transcriptase domain then uses the atgRNA (or pegRNA) to template reverse transcription of the desired edit, directly polymerizing DNA onto the nicked target DNA strand. The edited DNA strand replaces the original DNA strand, creating a heteroduplex containing one edited strand and one unedited strand. Afterward, optionally, the prime editor (PE) guides resolution of the heteroduplex to favor copying the edit onto the unedited strand, completing the process (typically achieved with a nickase gRNA). Other enzymes that can be used to nick or cut only a single strand of double stranded DNA includes a cleavase (e.g., cleavase I enzyme).

In some embodiments, an additional agent or agents may be added that improve the efficiency and outcome purity of the prime edit. In some embodiments, the agent may be chemical or biological and disrupt DNA mismatch repair (MMR) processes at or near the edit site (i.e., PE4 and PE5 and PEmax architecture by Chen et al. Cell, 184, 1-18, Oct. 28, 2021; Chen et al. is incorporated herein by reference). In typical embodiments, the agent is a MMR-inhibiting protein. In certain embodiments, the MMR-inhibiting protein is dominant negative MMR protein. In certain embodiments, the dominant negative MMR protein is MLH1dn. In particular embodiments, the MMR-inhibiting agent is incorporated into the co-delivery method described herein. In some embodiments, the MMR-inhibiting agent is linked or fused to the prime editor protein fusion, which may or may not have a linked or fused integrase. In some embodiments, the MMR-inhibiting agent is linked or fused to the Gene Writer™ protein, which may or may not have a linked or fused integrase.

The prime editor or gene editor system can be used to achieve DNA deletion and replacement. In some embodiments, the DNA deletion replacement is induced using a pair of atgRNAs or pegRNA that target opposite DNA strands, programming not only the sites that are nicked but also the outcome of the repair (i.e., PrimeDel by Choi et al. Nat. Biotechnology, Oct. 14, 2021; Choi et al. is incorporated herein by reference and TwinPE by Anzalone et al. BioRxiv, Nov. 2, 2021; Anzalone et al. is incorporated herein by reference). In some embodiments described herein, the DNA deletion is induced using a single atgRNA. In some embodiments, the DNA deletion and replacement is induced using a wild type Cas9 prime editor (PE-Cas9) system (i.e., PEDAR by Jiang et al. Nat. Biotechnology, Oct. 14, 2021; Jiang et al. is incorporated herein by reference in its entirety). In some embodiments, the DNA replacement is an integrase target recognition site or recombinase target recognition site. In certain embodiments, the constructs and methods described herein may be utilized to incorporate the pair of pegRNAs (or atgRNAs) used in PrimeDel, TwinPE (WO2021226558 incorporated by reference herein in its entirety), or PEDAR, the prime editor fusion protein or Gene Writer protein, optionally a nickase guide RNA (ngRNA), an integrase, a nucleic acid cargo, and optionally a recombinase into a LNP delivery system or vector delivery system (e.g., AAV or Adenovirus). The integrase may be directly linked, for example by a peptide linker, to the prime editor fusion or gene writer protein.

In some embodiments, the prime editors can refer to a retrovirus or lentivirus reverse transcriptase such as a Moloney Murine Leukemia Virus (M-MLV) reverse transcriptase (RT) fused to a CRISPR enzyme nickase such as a Cas9 H840A nickase, a Cas9nickase. In some embodiments, the prime editors can refer to a retrovirus or lentivirus reverse transcriptase such as a Moloney Murine Leukemia Virus (M-MLV) reverse transcriptase (RT) fused to a cleavase. In some embodiments the RT can be fused at, near or to the C-terminus of a Cas9nickase, e.g., Cas9 H840A. Fusing the RT to the C-terminus region, e.g., to the C-terminus, of the Cas9 nickase may result in higher editing efficiency. Such a complex is called PEI. In some embodiments, the CRISPR enzyme nickase, e.g., Cas9(H840A), i.e., a Cas9nickase, can be linked to a non-M-MLV reverse transcriptase such as an AMV-RT or XRT (Cas9(H840A)-AMV-RT or XRT). In some embodiments, instead of the CRISPR enzyme nickase being a Cas9 (H840A), i.e., instead of being a Cas9 nickase, the CRISPR enzyme nickase instead can be a CRISPR enzyme that naturally is a nickase or cuts a single strand of double stranded DNA; for instance, the CRISPR enzyme nickase can be Cas12a/b. Alternatively, the CRISPR enzyme nickase can be another mutation of Cas9, such as Cas9(D10A). A CRISPR enzyme, such as a CRISPR enzyme nickase, such as Cas9 (wild type), Cas9(H840A), Cas9(D10A) or Cas 12a/b nickase can be fused in some embodiments to a pentamutant of M-MLV RT (D200N/L603W/T330P/T306K/W313F), whereby there can be up to about 45-fold higher efficiency, and this is called PE2. In some embodiments, the M-MLV RT comprise one or more of the mutations Y8H, P51L, S56A, S67R, E69K, V129P, L139P, T197A, H204R, V223H, T246E, N249D, E286R, Q2911, E302K, E302R, F309N, M320L, P330E, L435G, L435R, N454K, D524A, D524G, D524N, E562Q, D583N, H594Q, E607K, D653N, and L671P. Specific M-MLV RT mutations are shown in Table 1.

TABLE 1

| | | Forward Sequence |
|---|---|---|
| SEQ ID NO | Description | (5′-3′) |
| SEQ ID NO: 01 | RT_mut_L139P | ttgagcgggCCC |
| | | ccaccgt |
| SEQ ID NO: 02 | RT_mut_E562Q | cagcgggctCAG |
| | | ctgatagca |
| SEQ ID NO: 03 | RT_mut_D653N | cggatggctAAC |
| | | caagcggcc |

In some embodiments, the reverse transcriptase can also be a wild-type or modified transcription xenopolymerase (RTX), avian myeloblastosis virus reverse transcriptase (AMV RT), Feline Immunodeficiency Virus reverse transcriptase (FIV-RT), FeLV-RT (Feline leukemia virus reverse transcriptase), HIV-RT (Human Immunodeficiency Virus reverse transcriptase). In some embodiments, the reverse transcriptase can be a fusion of MMuLV to the Sto7d DNA binding domain (see Ionnidi et al.; https://doi.org/10.1101/2021.11.01.466786). The fusion of MMuLV to the Sto7d DNA binding domain sequence is given in Table 2.

TABLE 2

| | | SEQ |
|---|---|---|
| | Forward Sequence | ID |
| Description | (5′-3′) | NO: |
| RT | atgactcactatcaggcctt | 4 |
| (1-478)_ | gcttttggacacggaccggg | |
| Sto7d | tccagttcggaccggtggta | |
| fusion | gccctgaacccggctacgct | |
| [MMulv | gctcccactgcctgaggaag | |
| sequence | ggctgcaacacaactgcctt | |
| (in | gatGGGACAGGTGGCGGTGG | |
| bold), | TGTCACCGTCAAGTTCAAGT | |
| Sto7d | ACAAGGGTGAGGAACTTGAA | |
| sequence] | GTTGATATTAGCAAAATCAA | |
| | GAAGGTTTGGCGCGTTGGTA | |
| | AAATGATATCTTTTACTTAT | |
| | GACGACAACGGCAAGACAGG | |
| | TAGAGGGGCAGTGTCTGAGA | |
| | AAGACGCCCCCAAGGAGCTG | |
| | TTGCAAATGTTGGAAAAGTC | |
| | TGGGAAAAAGtctggcggct | |
| | caaaaagaaccgccgacggc | |
| | agegaattcgagcccaagaa | |
| | gaagaggaaagtc | |

PE3, PE3b, PE4, PE5, and/or PEmax, which a skilled person can incorporate into the co-delivery system described herein, involves nicking the non-edited strand, potentially causing the cell to remake that strand using the edited strand as the template to induce HR. The nicking of the non-edited strand can involve the use of a nicking guide RNA (ngRNA).

The skilled person can readily incorporate into the co-delivery system described herein described herein a prime editing or CRISPR system. Examples of prime editors can be found in the following: WO2020/191153, WO2020/191171, WO2020/191233, WO2020/191234, WO2020/191239, WO2020/191241, WO2020/191242, WO2020/191243, WO2020/191245, WO2020/191246, WO2020/191248, WO2020/191249, each of which is incorporated by reference herein in its entirety. In addition, mention is made, and can be used herein, of CRISPR Patent Applications and Patents of the Zhang laboratory and/or Broad Institute, Inc. and Massachusetts Institute of Technology and/or Broad Institute, Inc., Massachusetts Institute of Technology and President and Fellows of Harvard College and/or Editas Medicine, Inc. Broad Institute, Inc., The University of Iowa Research Foundation and Massachusetts Institute of Technology, including those claiming priority to U.S. Application 61/736,527, filed Dec. 12, 2012, including U.S. Pat. Nos. 11,104,937, 11,091,798, 11,060,115, 11,041,173, 11,021,740, 11,008,588, 11,001,829, 10,968,257, 10,954,514, 10,946,108, 10,930,367, 10,876,100, 10,851,357, 10,781,444, 10,711,285, 10,689,691, 10,648,020, 10,640,788, 10,577,630, 10,550,372, 10,494,621, 10,377,998, 10,266,887, 10,266,886, 10,190,137, 9,840,713, 9,822,372, 9,790,490, 8,999,641, 8,993,233, 8,945,839, 8,932,814, 8,906,616, 8,895,308, 8,889,418, 8,889,356, 8,871,445, 8,865,406, 8,795,965, 8,771,945, and 8,697,359; CRISPR Patent Applications and Patents of the Doudna laboratory and/or of Regents of the University of California, the University of Vienna and Emmanuelle Charpentier, including those claiming priority to U.S. application 61/652,086, filed May 25, 2012, and/or 61/716,256, filed Oct. 19, 2012, and/or 61/757,640, filed Jan. 28, 2013, and/or 61/765,576, filed Feb. 15, 2013 and/or Ser. No. 13/842,859, including U.S. Pat. Nos. 11,028,412, 11,008,590, 11,008,589, 11,001,863, 10,988,782, 10,988,780, 10,982,231, 10,982,230, 10,900,054, 10,793,878, 10,774,344, 10,752,920, 10,676,759, 10,669,560, 10,640,791, 10,626,419, 10,612,045, 10,597,680, 10,577,631, 10,570,419, 10,563,227, 10,550,407, 10,533,190, 10,526,619, 10,519,467, 10,513,712, 10,487,341, 10,443,076, 10,428,352, 10,421,980, 10,415,061, 10,407,697, 10,400,253, 10,385,360, 10,358,659, 10,358,658, 10,351,878, 10,337,029, 10,308,961, 10,301,651, 10,266,850, 10,227,611, 10,113,167, and 10,000,772; CRISPR Patent Applications and Patents of Vilnius University and/or the Siksnys laboratory, including those claiming priority to U.S. application 62/046,384 and/or 61/625,420 and/or 61/613,373 and/or PCT/IB2015/056756, including U.S. Pat. No. 10,385,336; CRISPR Patent Applications and Patents of the President and Fellows of Harvard College, including those of George Church's laboratory and/or claiming priority to U.S. application 61/738,355, filed Dec. 17, 2012, including 11,111,521, 11,085,072, 11,064,684, 10,959,413, 10,925,263, 10,851,369, 10,787,684, 10,767,194, 10,717,990, 10,683,490, 10,640,789, 10,563,225, 10,435,708, 10,435,679, 10,375,938, 10,329,587, 10,273,501, 10,100,291, 9,970,024, 9,914,939, 9,777,262, 9,587,252, 9,267,135, 9,260,723, 9,074,199, 9,023,649; CRISPR Patent Applications and Patents of the President and Fellows of Harvard College, including those of David Liu's laboratory, including 11,111,472, 11,104,967, 11,078,469, 11,071,790, 11,053,481, 11,046,948, 10,954,548, 10,947,530, 10,912,833, 10,858,639, 10,745,677, 10,704,062, 10,682,410, 10,612,011, 10,597,679, 10,508,298, 10,465,176, 10,323,236, 10,227,581, 10,167,457, 10,113,163, 10,077,453, 9,999,671, 9,840,699, 9,737,604, 9,526,784, 9,388,430, 9,359,599, 9,340,800, 9,340,799, 9,322,037, 9,322,006, 9,228,207, 9,163,284, and 9,068,179; and CRISPR Patent Applications and Patents of Toolgen Incorporated and/or the Kim laboratory and/or claiming priority to U.S. application 61/717,324, filed Oct. 23, 2012 and/or 61/803,599, filed Mar. 20, 2013 and/or 61/837,481, filed Jun. 20, 2013 and/or 62/033,852, filed Aug. 6, 2014 and/or PCT/KR2013/009488 and/or PCT/KR2015/008269, including U.S. Pat. Nos. 10,851,380, and 10,519,454; and CRISPR Patent Applications and Patents of Sigma and/or Millipore and/or the Chen laboratory and/or claiming priority to U.S. application 61/734,256, filed Dec. 6, 2012 and/or 61/758,624, filed Jan. 30, 2013 and/or 61/761,046, filed Feb. 5, 2013 and/or 61/794,422, filed Mar. 15, 2013, including U.S. Pat. No. 10,731,181, each of which is hereby incorporated herein by reference, and from the disclosures of the foregoing, the skilled person can readily make and use a prime editing or CRISPR system, and can especially appreciate impaired endonucleases, such as a mutated Cas9 that only nicks a single strand of DNA and is hence a nickase, or a CRISPR enzyme that only makes a single-stranded cut that can be employed in a PASTE system of the invention. Further, from the disclosures of the foregoing, the skilled person can incorporate the selected CRISPR enzyme, as part of the prime editor fusion or gene editor fusion, into the co-delivery method described herein.

Prior to RT-mediated edit incorporation, the prime editor protein (or system) (1) site-specifically targets a genomic locus and (2) performs a catalytic cut or nick. These steps are typically performed by a CRISPR-Cas. However, in some embodiments the Cas protein may be substituted by other nucleic acid programmable DNA binding proteins (napDNAbp) such as zinc finger nucleases (ZFNs), transcription activator-like effector nucleases (TALENs), or meganucleases. In addition, to the extent the “targeting rules” of other napDNAbp are known or are newly determined, it becomes possible to use new napDNAbp, beyond Cas9, to site specifically target and modify genomic sites of interest.

Similar to a prime editor protein, a Gene Writer can introduce novel DNA elements, such as an integration target site, into a DNA locus. A Gene Writer protein comprises: (A) a polypeptide or a nucleic acid encoding a polypeptide, wherein the polypeptide comprises (i) a reverse transcriptase domain, and either (x) an endonuclease domain that contains DNA binding functionality or (y) an endonuclease domain and separate DNA binding domain; and (B) a template RNA comprising (i) a sequence that binds the polypeptide and (ii) a heterologous insert sequence. Examples of such Gene Writer™ proteins and related systems can be found in US20200109398, which is incorporated by reference herein in its entirety.

In some embodiments, the prime editor or Gene Writer protein fusion or prime editor protein linked or fused to an integrase is expressed as a split construct. In typical embodiments, the split construct in reconstituted in a cell. In some embodiments, the split construct can be fused or ligated via intein protein splicing. In some embodiments, the split construct can be reconstituted via protein-protein inter-molecular bonding and/or interactions. In some embodiments, the split construct can be reconstituted via chemical, biological, or environmental induced oligomerization. In certain embodiments, the split construct can be adapted into one or more delivery vectors described herein.

In some embodiments, an integrase or recombinase is directly linked or fused, for example by a peptide linker, which may be cleavable or non-cleavable, to the prime editor fusion protein (i.e., fused Cas9 nickase-reverse transcriptase) or Gene Writer protein. Suitable linkers, for example between the Cas9, RT, and integrase, may be selected from Table 3:

TABLE 3

| | | SEQ ID | | SEQ ID |
|---|---|---|---|---|
| | Sequence (5′-3′) | NO: | Amino acid sequence | NO: |
| A-P2A | GGAAGCGGAGCTACTAACTTC | 5 | GSGATNFSLLKQAG | 13 |
| | AGCCTGCTGAAGCAGGCTGGC | | DVEENPGP | |
| | GACGTGGAGGAGAACCCTGGA | | | |
| | CCT | | | |
| B- | GGGGGAGGAGGTTCTGGAGGC | 6 | GGGGSGGGGSGGGG | 14 |
| (GGGS)3 | GGAGGCTCCGGAGGCGGAGGG | | S | |
| | TCA | | | |
| C- | GGAGGTGGCGGGAGC | 7 | GGGGS | 15 |
| GGGGS | | | | |
| D- | CCCGCACCAGCGCCT | 8 | PAPAP | 16 |
| PAPAP | | | | |
| E- | GAGGCAGCTGCCAAGGAAGCC | 9 | EAAAKEAAAKEAAA | 17 |
| (EAAAK)3 | GCTGCCAAGGAGGCGGCCGCA | | K | |
| | AAG | | | |
| F-XTEN | AGTGGGAGCGAGACCCCTGGG | 10 | SGSETPGTSESATPES | 18 |
| | ACTAGCGAGTCAGCTACACCC | | | |
| | GAAAGC | | | |
| G- | GGGGGGTCAGGTGGATCCGGC | 11 | GGSGGSGGSGGSGG | 19 |
| (GGS)6 | GGAAGTGGCGGATCCGGTGGA | | SGGS | |
| | TCTGGCGGCAGT | | | |
| H- | GAAGCTGCTGCTAAG | 12 | EAAAK | 20 |
| EAAAK | | | | |
| (GGGGS)4 | GGCGGCGGCGGCAGCGGCGGC | 543 | GGGGSGGGGSGGGG | 551 |
| | GGCGGCAGCGGCGGCGGCGGC | | SGGGGS | |
| | AGCGGCGGCGGCGGCAGC | | | |
| PAS8 | GGCGGCGCGAGCCCGGCGGGC | 544 | GGASPAGG | 552 |
| | GGC | | | |
| PAS12 | GGCGGCGCGAGCCCGGCGGCG | 545 | GGASPAAPAPAG | 553 |
| | CCGGCGCCGGCGGGC | | | |
| A(EAAK) | GCGGAAGCGGCGAAAGAAGCG | 546 | AEAAKEAAKEAAKE | 554 |
| 4ALEA(E | GCGAAAGAAGCGGCGAAAGAA | | AAKALEAEAAAKEA | |
| AAAK)4A | GCGGCGAAAGCGCTGGAAGCG | | AAKEAAAKEAAAK | |
| | GAAGCGGCGGCGAAAGAAGCG | | A | |
| | GCGGCGAAAGAAGCGGCGGCG | | | |
| | AAAGAAGCGGCGGCGAAAGCG | | | |
| Camel | GCGCATCATAGCGAAGATCCG | 547 | AHHSEDPGGGGSGG | 555 |
| | GGCGGCGGCGGCAGCGGCGGC | | GGSGGGGS | |
| | GGCGGCAGCGGCGGCGGCGGC | | | |
| | AGC | | | |
| FRF | GGCGGCGGCGGCAGCGAAGCG | 548 | GGGGSEAAAKGGGG | 556 |
| | GCGGCGAAAGGCGGCGGCGGC | | S | |
| | AGC | | | |
| RFR | GAAGCGGCGGCGAAAGGCGGC | 549 | EAAAKGGGGSEAAA | 557 |
| | GGCGGCAGCGAAGCGGCGGCG | | K | |
| | AAA | | | |
| Modified | AGCGGCGGCAGCAGCGGCGGC | 550 | SGGSSGGSSGSETPG | 558 |
| XTEN | AGCAGCGGCAGCGAAACCCCG | | TSESATPESSGGSSG | |
| (mXTEN) | GGCACCAGCGAAAGCGCGACC | | GSST | |
| | CCGGAAAGCAGCGGCGGCAGC | | | |
| | AGCGGCGGCAGCAGCACC | | | |

In some embodiments, the prime editor or Gene Writer protein fusion or prime editor protein linked or fused to an integrase is expressed as a split construct. In typical embodiments, the split construct in reconstituted in a cell. In some embodiments, the split construct can be fused or ligated via intein protein splicing. In some embodiments, the split construct can be reconstituted via protein-protein inter-molecular bonding and/or interactions. In some embodiments, the split construct can be reconstituted via chemical, biological, or environmental induced oligomerization. In certain embodiments, the split construct can be adapted into one or more nucleic acid constructs described herein.

### 6.2. Type II CRISPR Proteins

The skilled person can incorporate a selected CRISPR enzyme, described below, as part of the prime editor fusion, into the co-delivery method described herein. Streptococcus pyogenes Cas9 (SpCas9), the most common enzyme used in genome-editing applications, is a large nuclease of 1368 amino acid residues. Advantages of SpCas9 include its short, 5′-NGG-3′ PAM and very high average editing efficiency. SpCas9 consists of two lobes: a recognition (REC) lobe and a nuclease (NUC) lobe. The REC lobe can be divided into three regions, a long a helix referred to as the bridge helix (residues 60-93), the REC1 (residues 94-179 and 308-713) domain, and the REC2 (residues 180-307) domain. The NUC lobe consists of the RuvC (residues 1-59, 718-769, and 909-1098), HNH (residues 775-908), and PAM-interacting (PI) (residues 1099-1368) domains. The negatively charged sgRNA:target DNA heteroduplex is accommodated in a positively charged groove at the interface between the REC and NUC lobes. In the NUC lobe, the RuvC domain is assembled from the three split RuvC motifs (RuvC I-III) and interfaces with the PI domain to form a positively charged surface that interacts with the 30 tail of the sgRNA. The HNH domain lies between the RuvC II-III motifs and forms only a few contacts with the rest of the protein. Structural aspects of SpCas9 are described by Nishimasu et al., Crystal Structure of Cas9 in Complex with Guide RNA and Target DNA, Cell 156, 935-949, Feb. 27, 2014.

REC lobe: The REC lobe includes the REC1 and REC2 domains. The REC2 domain does not contact the bound guide:target heteroduplex, indicating that truncation of REC lobe may be tolerated by SpCas9. Further, SpCas9 mutant lacking the REC2 domain (D175-307) retained ˜50% of the wild-type Cas9 activity, indicating that the REC2 domain is not critical for DNA cleavage. In striking contrast, the deletion of either the repeat-interacting region (D97-150) or the anti-repeat-interacting region (D312-409) of the REC1 domain abolished the DNA cleavage activity, indicating that the recognition of the repeat:anti-repeat duplex by the REC1 domain is critical for the Cas9 function.

PAM-Interacting domain: The NUC lobe contains the PAM-interacting (PI) domain that is positioned to recognize the PAM sequence on the noncomplementary DNA strand. The PI domain of SpCas9 is required for the recognition of 5′-NGG-3′ PAM, and deletion of the PI domain (A1099-1368) abolished the cleavage activity, indicating that the PI domain is critical for SpCas9 function and a major determinant for the PAM specificity.

RuvC domain: The RuvC nucleases of SpCas9 have an RNase H fold and four catalytic residues, Asp10 (Ala), Glu762, His983, and Asp986, that are critical for the two-metal cleavage of the noncomplementary strand of the target DNA. In addition to the conserved RNase H fold, the Cas9 RuvC domain has other structural elements involved in interactions with the guide:target heteroduplex (an end-capping loop between α42 and α43) and the PI domain/stem loop 3 (β hairpin formed by β3 and β4).

HNH domain: SpCas9 HNH nucleases have three catalytic residues, Asp839, His840, and Asn863 and cleave the complementary strand of the target DNA through a single-metal mechanism.

sgRNA:DNA recognition: The sgRNA guide region is primarily recognized by the REC lobe. The backbone phosphate groups of the guide region (nucleotides 2, 4-6, and 13-20) interact with the REC1 domain (Arg165, Gly166, Arg403, Asn407, Lys510, Tyr515, and Arg661) and the bridge helix (Arg63, Arg66, Arg70, Arg71, Arg74, and Arg78). The 20-hydroxyl groups of G1, C15, U16, and G19 hydrogen bond with Val1009, Tyr450, Arg447/Ile448, and Thr404, respectively.

A mutational analysis demonstrated that the R66A, R70A, and R74A mutations on the bridge helix markedly reduced the DNA cleavage activities, highlighting the functional significance of the recognition of the sgRNA “seed” region by the bridge helix. Although Arg78 and Arg165 also interact with the “seed” region, the R78A and R165A mutants showed only moderately decreased activities. These results are consistent with the fact that Arg66, Arg70, and Arg74 form multiple salt bridges with the sgRNA backbone, whereas Arg78 and Arg165 form a single salt bridge with the sgRNA backbone. Moreover, the alanine mutations of the repeat:anti-repeat duplex-interacting residues (Arg75 and Lys163) and the stemloop-1-interacting residue (Arg69) resulted in decreased DNA cleavage activity, confirming the functional importance of the recognition of the repeat:anti-repeat duplex and stem loop 1 by Cas9.

RNA-guided DNA targeting: SpCas9 recognizes the guide:target heteroduplex in a sequence-independent manner. The backbone phosphate groups of the target DNA (nucleotides 1, 9-11, 13, and 20) interact with the REC1 (Asn497, Trp659, Arg661, and Gln695), RuvC (Gln926), and PI (Glu1108) domains. The C2′ atoms of the target DNA (nucleotides 5, 7, 8, 11, 19, and 20) form van der Waals interactions with the REC1 domain (Leu169, Tyr450, Met495, Met694, and His698) and the RuvC domain (Ala728). The terminal base pair of the guide:target heteroduplex (G1:C20′) is recognized by the RuvC domain via end-capping interactions; the sgRNA G1 and target DNA C20′ nucleobases interact with the Tyr1013 and Val1015 side chains, respectively, whereas the 20-hydroxyl and phosphate groups of sgRNA G1 interact with Val1009 and Gln926, respectively.

Repeat:Anti-Repeat duplex recognition: The nucleobases of U23/A49 and A42/G43 hydrogen bond with the side chain of Arg1122 and the main-chain carbonyl group of Phe351, respectively. The nucleobase of the flipped U44 is sandwiched between Tyr325 and His328, with its N3 atom hydrogen bonded with Tyr325, whereas the nucleobase of the unpaired G43 stacks with Tyr359 and hydrogen bonds with Asp364.

The nucleobases of G21 and U50 in the G21:U50 wobble pair stack with the terminal C20:G10 pair in the guide:target heteroduplex and Tyr72 on the bridge helix, respectively, with the U50 O4 atom hydrogen bonded with Arg75. Notably, A51 adopts the syn conformation and is oriented in the direction opposite to U50. The nucleobase of A51 is sandwiched between Phe1105 and U63, with its N1, N6, and N7 atoms hydrogen bonded with G62, Gly1103, and Phe1105, respectively.

Stem-loop recognition: Stem loop 1 is primarily recognized by the REC lobe, together with the PI domain. The backbone phosphate groups of stem loop 1 (nucleotides 52, 53, and 59-61) interact with the REC1 domain (Leu455, Ser460, Arg467, Thr472, and Ile473), the PI domain (Lys1123 and Lys1124), and the bridge helix (Arg70 and Arg74), with the 20-hydroxyl group of G58 hydrogen bonded with Leu455. A52 interacts with Phe1105 through a face-to-edge p-p stacking interaction, and the flipped U59 nucleobase hydrogen bonds with Asn77.

The single-stranded linker and stem loops 2 and 3 are primarily recognized by the NUC lobe. The backbone phosphate groups of the linker (nucleotides 63-65 and 67) interact with the RuvC domain (Glu57, Lys742, and Lys1097), the PI domain (Thr1102), and the bridge helix (Arg69), with the 20-hydroxyl groups of U64 and A65 hydrogen bonded with Glu57 and His721, respectively. The C67 nucleobase forms two hydrogen bonds with Val1100.

Stem loop 2 is recognized by Cas9 via the interactions between the NUC lobe and the non-Watson-Crick A68:G81 pair, which is formed by direct (between the A68 N6 and G81 O6 atoms) and water-mediated (between the A68 N1 and G81 N1 atoms) hydrogen-bonding interactions. The A68 and G81 nucleobases contact Ser1351 and Tyr1356, respectively, whereas the A68:G81 pair interacts with Thr1358 via a water-mediated hydrogen bond. The 20-hydroxyl group of A68 hydrogen bonds with His1349, whereas the G81 nucleobase hydrogen bonds with Lys33.

Stem loop 3 interacts with the NUC lobe more extensively, as compared to stem loop 2. The backbone phosphate group of G92 interacts with the RuvC domain (Arg40 and Lys44), whereas the G89 and U90 nucleobases hydrogen bond with Gln1272 and Glu1225/Ala1227, respectively. The A88 and C91 nucleobases are recognized by Asn46 via multiple hydrogen-bonding interactions.

Cas9 proteins smaller than SpCas9 allow more efficient packaging of nucleic acids encoding CRISPR systems, e.g., Cas9 and sgRNA into one rAAV (“all-in-one-AAV”) particle. In addition, efficient packaging of CRISPR systems can be achieved in other viral vector systems (i.e., lentiviral, integration deficient lentiviral, hd-AAV, etc.) and non-viral vector systems (i.e., lipid nanoparticle). Small Cas9 proteins can be advantageous for multidomain-Cas-nuclease-based systems for prime editing. Well characterized smaller Cas9 proteins include Staphylococcus aureus (SauCas9, 1053 amino acid residues) and Campylobacter jejuni (CjCas9, 984 amino residues). However, both recognize longer PAMs, 5′-NNGRRT-3′ for SauCas9 (R=A or G) and 5′-NNNNRYAC-3′ for CjCas9 (Y=C or T), which reduces the number of uniquely addressable target sites in the genome, in comparison to the NGG SpCas9 PAM. Among smaller Cas9s, Schmidt et al. identified Staphylococcus lugdunensis (Slu) Cas9 as having genome-editing activity and provided homology mapping to SpCas9 and SauCas9 to facilitate generation of nickases and inactive (“dead”) enzymes (Schmidt et al., 2021, Improved CRISPR genome editing using small highly active and specific engineered RNA-guided nucleases. Nat Commun 12, 4219. doi.org/10.1038/s41467-021-24454-5) and engineered nucleases with higher cleavage activity by fragmenting and shuffling Cas9 DNAs. The small Cas9s and nickases are useful in the instant invention.

Besides dead Cas9 and Cas9 nickase variants, the Cas9 proteins used herein may also include other “Cas9 variants” having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SEQ ID NO: 18).

In some embodiments, the disclosure also may utilize Cas9 fragments that retain their functionality and that are fragments of any herein disclosed Cas9 protein. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In various embodiments, the prime editors disclosed herein may comprise one of the Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants.

TABLE 4
Cas9 orthologs

| | | |
|---|---|---|
| Streptococcus | MDKKYSIGLD IGTNSVGWAV ITDEYKVPSK KFKVLGNTDR HSIKKNLIGA | (SEQ |
| pyogenes | LLFDSGETAE ATRLKRTARR RYTRRKNRIC YLQEIFSNEM AKVDDSFFHR | ID |
| AJN60024.1 | LEESFLVEED KKHERHPIFG NIVDEVAYHE KYPTIYHLRK KLVDSTDKAD | NO: |
| GI: | LRLIYLALAH MIKFRGHFLI EGDLNPDNSD VDKLFIQLVQ TYNQLFEENP | 21) |
| 757015980 | INASGVDAKA ILSARLSKSR RLENLIAQLP GEKKNGLFGN LIALSLGLTP | |
| WP_010922251.1 | NFKSNFDLAE DAKLQLSKDT YDDDLDNLLA QIGDQYADLF LAAKNLSDAI | |
| | LLSDILRVNT EITKAPLSAS MIKRYDEHHQ DLTLLKALVR QQLPEKYKEI | |
| | FFDQSKNGYA GYIDGGASQE EFYKFIKPIL EKMDGTEELL VKLNREDLLR | |
| | KQRTFDNGSI PHQIHLGELH AILRRQEDFY PFLKDNREKI EKILTFRIPY | |
| | YVGPLARGNS RFAWMTRKSE ETITPWNFEE VVDKGASAQS FIERMTNEDK | |
| | NLPNEKVLPK HSLLYEYFTV YNELTKVKYV TEGMRKPAFL SGEQKKAIVD | |
| | LLFKTNRKVT VKQLKEDYFK KIECFDSVEI SGVEDRFNAS LGTYHDLLKI | |
| | IKDKDFLDNE ENEDILEDIV LTLTLFEDRE MIEERLKTYA HLFDDKVMKQ | |
| | LKRRRYTGWG RLSRKLINGI RDKQSGKTIL DFLKSDGFAN RNFMQLIHDD | |
| | SLTFKEDIQK AQVSGQGDSL HEHIANLAGS PAIKKGILQT VKVVDELVKV | |
| | MGRHKPENIV IEMARENQTT QKGQKNSRER MKRIEEGIKE LGSQILKEHP | |
| | VENTQLQNEK LYLYYLQNGR DMYVDQELDI NRLSDYDVDH IVPQSFLKDD | |
| | SIDNKVLTRS DKNRGKSDNV PSEEVVKKMK NYWRQLLNAK LITQRKEDNL | |
| | TKAERGGLSE LDKAGFIKRQ LVETRQITKH VAQILDSRMN TKYDENDKLI | |
| | REVKVITLKS KLVSDFRKDF QFYKVREINN YHHAHDAYLN AVVGTALIKK | |
| | YPKLESEFVY GDYKVYDVRK MIAKSEQEIG KATAKYFFYS NIMNFFKTEI | |
| | TLANGEIRKR PLIETNGETG EIVWDKGRDF ATVRKVLSMP QVNIVKKTEV | |
| | QTGGFSKESI LPKRNSDKLI ARKKDWDPKK YGGFDSPTVA YSVLVVAKVE | |
| | KGKSKKLKSV KELLGITIME RSSFEKNPID FLEAKGYKEV KKDLIIKLPK | |
| | YSLFELENGR KRMLASAGEL QKGNELALPS KYVNFLYLAS HYEKLKGSPE | |
| | DNEQKQLFVE QHKHYLDEII EQISEFSKRV ILADANLDKV LSAYNKHRDK | |
| | PIREQAENII HLFTLINLGA PAAFKYFDTT IDRKRYTSTK EVLDATLIHQ | |
| | SITGLYETRI DLS | |
| AJN60021.1 | MKRNYILGLD IGITSVGYGI IDYETRDVID AGVRLFKEAN VENNEGRRSK | (SEQ |
| GI: | RGARRLKRRR RHRIQRVKKL LFDYNLLTDH SELSGINPYE ARVKGLSQKL | ID |
| 757015977 | SEEEFSAALL HLAKRRGVHN VNEVEEDTGN ELSTKEQISR NSKALEEKYV | NO: |
| J7RUA5.1 | AELQLERLKK DGEVRGSINR FKTSDYVKEA KQLLKVQKAY HQLDQSFIDT | 22) |
| WP_053019794.1 | YIDLLETRRT YYEGPGEGSP FGWKDIKEWY EMLMGHCTYF PEELRSVKYA | |
| Staphylococcus | YNADLYNALN DLNNLVITRD ENEKLEYYEK FQIIENVFKQ KKKPTLKQIA | |
| aureus | KEILVNEEDI KGYRVTSTGK PEFTNLKVYH DIKDITARKE IIENAELLDQ | |
| | IAKILTIYQS SEDIQEELIN LNSELTQEEI EQISNLKGYT GTHNLSLKAI | |
| | NLILDELWHT NDNQIAIFNR LKLVPKKVDL SQQKEIPTTL VDDFILSPVV | |
| | KRSFIQSIKV INAIIKKYGL PNDIIIELAR EKNSKDAQKM INEMQKRNRQ | |
| | TNERIEEIIR TTGKENAKYL IEKIKLHDMQ EGKCLYSLEA IPLEDLLNNP | |
| | FNYEVDHIIP RSVSFDNSEN NKVLVKQEEN SKKGNRTPFQ YLSSSDSKIS | |
| | YETFKKHILN LAKGKGRISK TKKEYLLEER DINRFSVQKD FINRNLVDTR | |
| | YATRGLMNLL RSYFRVNNLD VKVKSINGGF TSFLRRKWKF KKERNKGYKH | |
| | HAEDALIIAN ADFIFKEWKK LDKAKKVMEN QMFEEKQAES MPEIETEQEY | |
| | KEIFITPHQI KHIKDFKDYK YSHRVDKKPN RELINDTLYS TRKDDKGNTL | |
| | IVNNLNGLYD KDNDKLKKLI NKSPEKLLMY HHDPQTYQKL KLIMEQYGDE | |
| | KNPLYKYYEE TGNYLTKYSK KDNGPVIKKI KYYGNKLNAH LDITDDYPNS | |
| | RNKVVKLSLK PYRFDVYLDN GVYKFVTVKN LDVIKKENYY EVNSKCYEEA | |
| | KKLKKISNQA EFIASFYNND LIKINGELYR VIGVNNDLLN RIEVNMIDIT | |
| | YREYLENMND KRPPRIIKTI ASKTQSIKKY STDILGNLYE VKSKKHPQII | |
| | KKG | |
| AJN60008.1 | MARILAFDIG ISSIGWAFSE NDELKDCGVR IFTKVENPKT GESLALPRRL | (SEQ |
| GI: | ARSARKRLAR RKARLNHLKH LIANEFKLNY EDYQSFDESL AKAYKGSLIS | ID |
| 757015964 | PYELRFRALN ELLSKQDFAR VILHIAKRRG YDDIKNSDDK EKGAILKAIK | NO: |
| WP_002864485.1 | QNEEKLANYQ SVGEYLYKEY FQKFKENSKE FTNVRNKKES YERCIAQSFL | 23) |
| Campylobacter | KDELKLIFKK QREFGFSFSK KFEEEVLSVA FYKRALKDFS HLVGNCSFFT | |
| jejuni | DEKRAPKNSP LAFMFVALTR IINLLNNLKN TEGILYTKDD LNALLNEVLK | |
| subsp. jejuni | NGTLTYKQTK KLLGLSDDYE FKGEKGTYFI EFKKYKEFIK ALGEHNLSQD | |
| NCTC | DLNEIAKDIT LIKDEIKLKK ALAKYDLNQN QIDSLSKLEF KDHLNISFKA | |
| 11168 = | LKLVTPLMLE GKKYDEACNE LNLKVAINED KKDFLPAFNE TYYKDEVINP | |
| ATCC | VVLRAIKEYR KVLNALLKKY GKVHKINIEL AREVGKNHSQ RAKIEKEQNE | |
| 700819 | NYKAKKDAEL ECEKLGLKIN SKNILKLRLF KEQKEFCAYS GEKIKISDLQ | |
| | DEKMLEIDHI YPYSRSFDDS YMNKVLVFTK QNQEKLNQTP FEAFGNDSAK | |
| | WQKIEVLAKN LPTKKQKRIL DKNYKDKEQK NEKDRNLNDT RYIARLVLNY | |
| | TKDYLDFLPL SDDENTKLND TQKGSKVHVE AKSGMLTSAL RHTWGFSAKD | |
| | RNNHLHHAID AVIIAYANNS IVKAFSDEKK EQESNSAELY AKKISELDYK | |
| | NKRKFFEPFS GFRQKVLDKI DEIFVSKPER KKPSGALHEE TERKEEEFYQ | |
| | SYGGKEGVLK ALELGKIRKV NGKIVKNGDM FRVDIFKHKK TNKFYAVPIY | |
| | TMDFALKVLP NKAVARSKKG EIKDWILMDE NYEFCESLYK DSLILIQTKD | |
| | MQEPEFVYYN AFTSSTVSLI VSKHDNKFET LSKNQKILFK NANEKEVIAK | |
| | SIGIQNLKVF EKYIVSALGE VTKAEFRQRE DEKK | |
| Streptococcus | MSDLVLGLDI GIGSVGVGIL NKVTGEIIHK NSRIFPAAQA ENNLVRRTNR | (SEQ |
| thermophilus | QGRRLARRKK HRRVRLNRLF EESGLITDFT KISININPYQ LRVKGLTDEL | ID |
| LMD-9 | SNEELFIALK NMVKHRGISY LDDASDDGNS SVGDYAQIVK ENSKQLETKT | NO: |
| AJN60026.1 | PGQIQLERYQ TYGQLRGDET VEKDGKKHRL INVFPTSAYR SEALRILQTQ | 24) |
| GI: | QEFNPQITDE FINRYLEILT GKRKYYHGPG NEKSRTDYGR YRTSGETLDN | |
| 757015982 | IFGILIGKCT FYPDEFRAAK ASYTAQEFNL LNDLNNLTVP TETKKLSKEQ | |
| WP_011680957.1 | KNQIINYVKN EKAMGPAKLF KYIAKLLSCD VADIKGYRID KSGKAEIHTF | |
| | EAYRKMKTLE TLDIEQMDRE TLDKLAYVLT LNTEREGIQE ALEHEFADGS | |
| | FSQKQVDELV QFRKANSSIF GKGWHNFSVK LMMELIPELY ETSEEQMTIL | |
| | TRLGKQKTTS SSNKTKYIDE KLLTEEIYNP VVAKSVRQAI KIVNAAIKEY | |
| | GDFDNIVIEM ARETNEDDEK KAIQKIQKAN KDEKDAAMLK AANQYNGKAE | |
| | LPHSVFHGHK QLATKIRLWH QQGERCLYTG KTISIHDLIN NSNQFEVDHI | |
| | LPLSITFDDS LANKVLVYAT ANQEKGQRTP YQALDSMDDA WSFRELKAFV | |
| | RESKTLSNKK KEYLLTEEDI SKFDVRKKFI ERNLVDTRYA SRVVLNALQE | |
| | HFRAHKIDTK VSVVRGQFTS QLRRHWGIEK TRDTYHHHAV DALIIAASSQ | |
| | LNLWKKQKNT LVSYSEDQLL DIETGELISD DEYKESVFKA PYQHFVDTLK | |
| | SKEFEDSILF SYQVDSKENR KISDATIYAT RQAKVGKDKA DETYVLGKIK | |
| | DIYTQDGYDA FMKIYKKDKS KFLMYRHDPQ TFEKVIEPIL ENYPNKQINE | |
| | KGKEVPCNPF LKYKEEHGYI RKYSKKGNGP EIKSLKYYDS KLGNHIDITP | |
| | KDSNNKVVLQ SVSPWRADVY FNKTTGKYEI LGLKYADLQF EKGTGTYKIS | |
| | QEKYNDIKKK EGVDSDSEFK FTLYKNDLLL VKDTETKEQQ LFRFLSRTMP | |
| | KQKHYVELKP YDKQKFEGGE ALIKVLGNVA NSGQCKKGLG KSNISIYKVR | |
| | TDVLGNQHII KNEGDKPKLD F | |
| Parvibaculum | MERIFGFDIG TTSIGFSVID YSSTQSAGNI QRLGVRIFPE ARDPDGTPLN | (SEQ |
| lavamentivorans | QQRRQKRMMR RQLRRRRIRR KALNETLHEA GFLPAYGSAD WPVVMADEPY | ID |
| DS-1 | ELRRRGLEEG LSAYEFGRAI YHLAQHRHFK GRELEESDTP DPDVDDEKEA | NO: |
| AJN60020.1 | ANERAATLKA LKNEQTTLGA WLARRPPSDR KRGIHAHRNV VAEEFERLWE | 25) |
| GI: | VQSKFHPALK SEEMRARISD TIFAQRPVFW RKNTLGECRF MPGEPLCPKG | |
| 757015976 | SWLSQQRRML EKLNNLAIAG GNARPLDAEE RDAILSKLQQ QASMSWPGVR | |
| WP_011995013.1 | SALKALYKQR GEPGAEKSLK FNLELGGESK LLGNALEAKL ADMFGPDWPA | |
| | HPRKQEIRHA VHERLWAADY GETPDKKRVI ILSEKDRKAH REAAANSEVA | |
| | DFGITGEQAA QLQALKLPTG WEPYSIPALN LFLAELEKGE RFGALVNGPD | |
| | WEGWRRINFP HRNQPTGEIL DKLPSPASKE ERERISQLRN PTVVRTQNEL | |
| | RKVVNNLIGL YGKPDRIRIE VGRDVGKSKR EREEIQSGIR RNEKQRKKAT | |
| | EDLIKNGIAN PSRDDVEKWI LWKEGQERCP YTGDQIGENA LFREGRYEVE | |
| | HIWPRSRSFD NSPRNKTLCR KDVNIEKGNR MPFEAFGHDE DRWSAIQIRL | |
| | QGMVSAKGGT GMSPGKVKRF LAKTMPEDFA ARQLNDTRYA AKQILAQLKR | |
| | LWPDMGPEAP VKVEAVTGQV TAQLRKLWTL NNILADDGEK TRADHRHHAI | |
| | DALTVACTHP GMTNKLSRYW QLRDDPRAEK PALTPPWDTI RADAEKAVSE | |
| | IVVSHRVRKK VSGPLHKETT YGDTGTDIKT KSGTYRQFVT RKKIESLSKG | |
| | ELDEIRDPRI KEIVAAHVAG RGGDPKKAFP PYPCVSPGGP EIRKVRLTSK | |
| | QQLNLMAQTG NGYADLGSNH HIAIYRLPDG KADFEIVSLF DASRRLAQRN | |
| | PIVQRTRADG ASFVMSLAAG EAIMIPEGSK KGIWIVQGVW ASGQVVLERD | |
| | TDADHSTTTR PMPNPILKDD AKKVSIDPIG RVRPSND | |
| Corynebacterium | MKYHVGIDVG TFSVGLAAIE VDDAGMPIKT LSLVSHIHDS GLDPDEIKSA | (SEQ |
| diphtheriae | VTRLASSGIA RRTRRLYRRK RRRLQQLDKF IQRQGWPVIE LEDYSDPLYP | ID |
| NCTC | WKVRAELAAS YIADEKERGE KLSVALRHIA RHRGWRNPYA KVSSLYLPDG | NO: |
| 13129 | PSDAFKAIRE EIKRASGQPV PETATVGQMV TLCELGTLKL RGEGGVLSAR | 26) |
| AJN60012.1 | LQQSDYAREI QEICRMQEIG QELYRKIIDV VFAAESPKGS ASSRVGKDPL | |
| GI: | QPGKNRALKA SDAFQRYRIA ALIGNLRVRV DGEKRILSVE EKNLVEDHLV | |
| 757015968 | NLTPKKEPEW VTIAEILGID RGQLIGTATM TDDGERAGAR PPTHDTNRSI | |
| WP_010933968.1 | VNSRIAPLVD WWKTASALEQ HAMVKALSNA EVDDFDSPEG AKVQAFFADL | |
| | DDDVHAKLDS LHLPVGRAAY SEDTLVRLTR RMLSDGVDLY TARLQEFGIE | |
| | PSWTPPTPRI GEPVGNPAVD RVLKTVSRWL ESATKTWGAP ERVIIEHVRE | |
| | GFVTEKRARE MDGDMRRRAA RNAKLFQEMQ EKLNVQGKPS RADLWRYQSV | |
| | QRQNCQCAYC GSPITFSNSE MDHIVPRAGQ GSTNTRENLV AVCHRCNQSK | |
| | GNTPFAIWAK NTSIEGVSVK EAVERTRHWV TDTGMRSTDF KKFTKAVVER | |
| | FQRATMDEEI DARSMESVAW MANELRSRVA QHFASHGTTV RVYRGSLTAE | |
| | ARRASGISGK LKFFDGVGKS RLDRRHHAID AAVIAFTSDY VAETLAVRSN | |
| | LKQSQAHRQE APQWREFTGK DAEHRAAWRV WCQKMEKLSA LLTEDLRDDR | |
| | VVVMSNVRLR LGNGSAHKET IGKLSKVKLS SQLSVSDIDK ASSEALWCAL | |
| | TREPGFDPKE GLPANPERHI RVNGTHVYAG DNIGLFPVSA GSIALRGGYA | |
| | ELGSSFHHAR VYKITSGKKP AFAMLRVYTI DLLPYRNQDL FSVELKPQTM | |
| | SMRQAEKKLR DALATGNAEY LGWLVVDDEL VVDTSKIATD QVKAVEAELG | |
| | TIRRWRVDGF FSPSKLRLRP LQMSKEGIKK ESAPELSKII DRPGWLPAVN | |
| | KLFSDGNVTV VRRDSLGRVR LESTAHLPVT WKVQ | |
| Streptococcus | MTNGKILGLD IGIASVGVGI IEAKTGKVVH ANSRLFSAAN AENNAERRGE | (SEQ |
| pasteurianus | RGSRRLNRRK KHRVKRVRDL FEKYGIVTDF RNLNLNPYEL RVKGLTEQLK | ID |
| WP_013852048.1 | NEELFAALRT ISKRRGISYL DDAEDDSTGS TDYAKSIDEN RRLLKNKTPG | NO: |
| | QIQLERLEKY GQLRGNFTVY DENGEAHRLI NVESTSDYEK EARKILETQA | 27) |
| | DYNKKITAEF IDDYVEILTQ KRKYYHGPGN EKSRTDYGRF RTDGTTLENI | |
| | FGILIGKCNF YPDEYRASKA SYTAQEYNFL NDLNNLKVST ETGKLSTEQK | |
| | ESLVEFAKNT ATLGPAKLLK EIAKILDCKV DEIKGYREDD KGKPDLHTFE | |
| | PYRKLKFNLE SINIDDLSRE VIDKLADILT LNTEREGIED AIKRNLPNQF | |
| | TEEQISEIIK VRKSQSTAFN KGWHSFSAKL MNELIPELYA TSDEQMTILT | |
| | RLEKFKVNKK SSKNTKTIDE KEVTDEIYNP VVAKSVRQTI KIINAAVKKY | |
| | GDFDKIVIEM PRDKNADDEK KFIDKRNKEN KKEKDDALKR AAYLYNSSDK | |
| | LPDEVFHGNK QLETKIRLWY QQGERCLYSG KPISIQELVH NSNNFEIDHI | |
| | LPLSLSFDDS LANKVLVYAW TNQEKGQKTP YQVIDSMDAA WSFREMKDYV | |
| | LKQKGLGKKK RDYLLTTENI DKIEVKKKFI ERNLVDTRYA SRVVLNSLQS | |
| | ALRELGKDTK VSVVRGQFTS QLRRKWKIDK SRETYHHHAV DALIIAASSQ | |
| | LKLWEKQDNP MFVDYGKNQV VDKQTGEILS VSDDEYKELV FQPPYQGFVN | |
| | TISSKGFEDE ILFSYQVDSK YNRKVSDATI YSTRKAKIGK DKKEETYVLG | |
| | KIKDIYSQNG FDTFIKKYNK DKTQFLMYQK DSLTWENVIE VILRDYPTTK | |
| | KSEDGKNDVK CNPFEEYRRE NGLICKYSKK GKGTPIKSLK YYDKKLGNCI | |
| | DITPEESRNK VILQSINPWR ADVYFNPETL KYELMGLKYS DLSFEKGTGN | |
| | YHISQEKYDA IKEKEGIGKK SEFKFTLYRN DLILIKDIAS GEQEIYRFLS | |
| | RTMPNVNHYV ELKPYDKEKF DNVQELVEAL GEADKVGRCI KGLNKPNISI | |
| | YKVRTDVLGN KYFVKKKGDK PKLDFKNNK K | |
| Neisseria | MAAFKPNPMN YILGLDIGIA SVGWAIVEID EEENPIRLID LGVRVFERAE | (SEQ |
| cinerea | VPKTGDSLAA ARRLARSVRR LTRRRAHRLL RARRLLKREG VLQAADFDEN | ID |
| ATCC | GLIKSLPNTP WQLRAAALDR KLTPLEWSAV LLHLIKHRGY LSQRKNEGET | NO: |
| 14685 | ADKELGALLK GVADNTHALQ TGDFRTPAEL ALNKFEKESG HIRNQRGDYS | 28) |
| AJN60019.1 | HTFNRKDLQA ELNLLFEKQK EFGNPHVSDG LKEGIETLLM TQRPALSGDA | |
| GI: | VQKMLGHCTF EPTEPKAAKN TYTAERFVWL TKLNNLRILE QGSERPLTDT | |
| 757015975 | ERATLMDEPY RKSKLTYAQA RKLLDLDDTA FFKGLRYGKD NAEASTLMEM | |
| WP_003676410.1 | KAYHAISRAL EKEGLKDKKS PLNLSPELQD EIGTAFSLFK TDEDITGRLK | |
| | DRVQPEILEA LLKHISFDKF VQISLKALRR IVPLMEQGNR YDEACTEIYG | |
| | DHYGKKNTEE KIYLPPIPAD EIRNPVVLRA LSQARKVING VVRRYGSPAR | |
| | IHIETAREVG KSFKDRKEIE KRQEENRKDR EKSAAKFREY FPNFVGEPKS | |
| | KDILKLRLYE QQHGKCLYSG KEINLGRLNE KGYVEIDHAL PFSRTWDDSF | |
| | NNKVLALGSE NQNKGNQTPY EYENGKDNSR EWQEFKARVE TSRFPRSKKQ | |
| | RILLQKFDED GFKERNLNDT RYINRFLCQF VADHMLLTGK GKRRVFASNG | |
| | QITNLLRGFW GLRKVRAEND RHHALDAVVV ACSTIAMQQK ITRFVRYKEM | |
| | NAFDGKTIDK ETGEVLHQKA HFPQPWEFFA QEVMIRVFGK PDGKPEFEEA | |
| | DTPEKLRTLL AEKLSSRPEA VHKYVTPLFI SRAPNRKMSG QGHMETVKSA | |
| | KRLDEGISVL RVPLTQLKLK DLEKMVNRER EPKLYEALKA RLEAHKDDPA | |
| | KAFAEPFYKY DKAGNRTQQV KAVRVEQVQK TGVWVHNHNG IADNATIVRV | |
| | DVFEKGGKYY LVPIYSWQVA KGILPDRAVV QGKDEEDWTV MDDSFEFKFV | |
| | LYANDLIKLT AKKNEFLGYF VSLNRATGAI DIRTHDTDST KGKNGIFQSV | |
| | GVKTALSFQK YQIDELGKEI RPCRLKKRPP VR | |
| AJN60009.1 | MSDLVLGLDI GIGSVGVGIL NKVTGEIIHK NSRIFPAAQA ENNLVRRTNR | (SEQ |
| GI: | QGRRLARRKK HRRVRLNRLF EESGLITDFT KISININPYQ LRVKGLTDEL | ID |
| 757015965 | SNEELFIALK NMVKHRGISY LDDASDDGNS SVGDYAQIVK ENSKQLETKT | NO: |
| St1Cas9 + | PGQIQLERYQ TYGQLRGDFT VEKDGKKHRL INVFPTSAYR SEALRILQTQ | 29) |
| SpCas9 | QEFNPQITDE FINRYLEILT GKRKYYHGPG NEKSRTDYGR YRTSGETLDN | |
| | IFGILIGKCT FYPDEFRAAK ASYTAQEFNL LNDLNNLTVP TETKKLSKEQ | |
| | KNQIINYVKN EKAMGPAKLF KYIAKLLSCD VADIKGYRID KSGKAEIHTF | |
| | EAYRKMKTLE TLDIEQMDRE TLDKLAYVLT LNTEREGIQE ALEHEFADGS | |
| | FSQKQVDELV QFRKANSSIF GKGWHNFSVK LMMELIPELY ETSEEQMTIL | |
| | TRLGKQKTTS SSNKTKYIDE KLLTEEIYNP VVAKSVRQAI KIVNAAIKEY | |
| | GDFDNIVIEM ARENQTTQKG QKNSRERMKR IEEGIKELGS QILKEHPVEN | |
| | TQLQNEKLYL YYLQNGRDMY VDQELDINRL SDYDVDHIVP QSFLKDDSID | |
| | NKVLTRSDKN RGKSDNVPSE EVVKKMKNYW RQLLNAKLIT QRKFDNLTKA | |
| | ERGGLSELDK AGFIKRQLVE TRQITKHVAQ ILDSRMNTKY DENDKLIREV | |
| | KVITLKSKLV SDFRKDFQFY KVREINNYHH AHDAYLNAVV GTALIKKYPK | |
| | LESEFVYGDY KVYDVRKMIA KSEQEIGKAT AKYFFYSNIM NFFKTEITLA | |
| | NGEIRKRPLI ETNGETGEIV WDKGRDFATV RKVLSMPQVN IVKKTEVQTG | |
| | GFSKESILPK RNSDKLIARK KDWDPKKYGG FDSPTVAYSV LVVAKVEKGK | |
| | SKKLKSVKEL LGITIMERSS FEKNPIDFLE AKGYKEVKKD LIIKLPKYSL | |
| | FELENGRKRM LASAGELQKG NELALPSKYV NFLYLASHYE KLKGSPEDNE | |
| | QKQLFVEQHK HYLDEIIEQI SEFSKRVILA DANLDKVLSA YNKHRDKPIR | |
| | EQAENIIHLF TLINLGAPAA FKYFDTTIDR KRYTSTKEVL DATLIHQSIT | |
| | GLYETRIDLS QLGGD | |
| Campylobacter | MRILGFDIGI NSIGWAFVEN DELKDCGVRI FTKAENPKNK ESLALPRRNA | (SEQ |
| lari Cas9 | RSSRRRLKRR KARLIAIKRI LAKELKLNYK DYVAADGELP KAYEGSLASV | ID |
| BAK69486.1 | YELRYKALTQ NLETKDLARV ILHIAKHRGY MNKNEKKSND AKKGKILSAL | NO: |
| | KNNALKLENY QSVGEYFYKE FFQKYKKNTK NFIKIRNTKD NYNNCVLSSD | 30) |
| | LEKELKLILE KQKEFGYNYS EDFINEILKV AFFQRPLKDF SHLVGACTFF | |
| | EEEKRACKNS YSAWEFVALT KIINEIKSLE KISGEIVPTQ TINEVLNLIL | |
| | DKGSITYKKF RSCINLHESI SFKSLKYDKE NAENAKLIDF RKLVEFKKAL | |
| | GVHSLSRQEL DQISTHITLI KDNVKLKTVL EKYNLSNEQI NNLLEIEFND | |
| | YINLSFKALG MILPLMREGK RYDEACEIAN LKPKTVDEKK DELPAFCDSI | |
| | FAHELSNPVV NRAISEYRKV LNALLKKYGK VHKIHLELAR DVGLSKKARE | |
| | KIEKEQKENQ AVNAWALKEC ENIGLKASAK NILKLKLWKE QKEICIYSGN | |
| | KISIEHLKDE KALEVDHIYP YSRSFDDSFI NKVLVETKEN QEKLNKTPFE | |
| | AFGKNIEKWS KIQTLAQNLP YKKKNKILDE NFKDKQQEDF ISRNLNDTRY | |
| | IATLIAKYTK EYLNFLLLSE NENANLKSGE KGSKIHVQTI SGMLTSVLRH | |
| | TWGFDKKDRN NHLHHALDAI IVAYSINSII KAFSDFRKNQ ELLKARFYAK | |
| | ELTSDNYKHQ VKFFEPFKSF REKILSKIDE IFVSKPPRKR ARRALHKDTF | |
| | HSENKIIDKC SYNSKEGLQI ALSCGRVRKI GTKYVENDTI VRVDIFKKQN | |
| | KFYAIPIYAM DFALGILPNK IVITGKDKNN NPKQWQTIDE SYEFCESLYK | |
| | NDLILLQKKN MQEPEFAYYN DESISTSSIC VEKHDNKFEN LTSNQKLLES | |
| | NAKEGSVKVE SLGIQNLKVF EKYIITPLGD KIKADFQPRE NISLKTSKKY | |
| | GLR | |
| AJN60010.1 | MDKKYSIGLD IGTNSVGWAV ITDEYKVPSK KFKVLGNTDR HSIKKNLIGA | (SEQ |
| GI: | LLFDSGETAE ATRLKRTARR RYTRRKNRIC YLQEIFSNEM AKVDDSFFHR | ID |
| 757015966 | LEESFLVEED KKHERHPIFG NIVDEVAYHE KYPTIYHLRK KLVDSTDKAD | NO: |
| SpCas9 + | LRLIYLALAH MIKFRGHFLI EGDLNPDNSD VDKLFIQLVQ TYNQLFEENP | 31) |
| St1Cas9 | INASGVDAKA ILSARLSKSR RLENLIAQLP GEKKNGLFGN LIALSLGLTP | |
| | NFKSNFDLAE DAKLQLSKDT YDDDLDNLLA QIGDQYADLF LAAKNLSDAI | |
| | LLSDILRVNT EITKAPLSAS MIKRYDEHHQ DLTLLKALVR QQLPEKYKEI | |
| | FFDQSKNGYA GYIDGGASQE EFYKFIKPIL EKMDGTEELL VKLNREDLLR | |
| | KQRTFDNGSI PHQIHLGELH AILRRQEDFY PFLKDNREKI EKILTFRIPY | |
| | YVGPLARGNS RFAWMTRKSE ETITPWNFEE VVDKGASAQS FIERMTNEDK | |
| | NLPNEKVLPK HSLLYEYFTV YNELTKVKYV TEGMRKPAFL SGEQKKAIVD | |
| | LLFKTNRKVT VKQLKEDYFK KIECFDSVEI SGVEDRENAS LGTYHDLLKI | |
| | IKDKDFLDNE ENEDILEDIV LTLTLFEDRE MIEERLKTYA HLEDDKVMKQ | |
| | LKRRRYTGWG RLSRKLINGI RDKQSGKTIL DELKSDGFAN RNFMQLIHDD | |
| | SLTFKEDIQK AQVSGQGDSL HEHIANLAGS PAIKKGILQT VKVVDELVKV | |
| | MGRHKPENIV IEMARETNED DEKKAIQKIQ KANKDEKDAA MLKAANQYNG | |
| | KAELPHSVFH GHKQLATKIR LWHQQGERCL YTGKTISIHD LINNSNQFEV | |
| | DHILPLSITF DDSLANKVLV YATANQEKGQ RTPYQALDSM DDAWSFRELK | |
| | AFVRESKTLS NKKKEYLLTE EDISKEDVRK KFIERNLVDT RYASRVVLNA | |
| | LQEHFRAHKI DTKVSVVRGQ FTSQLRRHWG IEKTRDTYHH HAVDALIIAA | |
| | SSQLNLWKKQ KNTLVSYSED QLLDIETGEL ISDDEYKESV FKAPYQHFVD | |
| | TLKSKEFEDS ILFSYQVDSK FNRKISDATI YATRQAKVGK DKADETYVLG | |
| | KIKDIYTQDG YDAFMKIYKK DKSKFLMYRH DPQTFEKVIE PILENYPNKQ | |
| | INEKGKEVPC NPFLKYKEEH GYIRKYSKKG NGPEIKSLKY YDSKLGNHID | |
| | ITPKDSNNKV VLQSVSPWRA DVYENKTTGK YEILGLKYAD LQFEKGTGTY | |
| | KISQEKYNDI KKKEGVDSDS EFKFTLYKND LLLVKDTETK EQQLFRFLSR | |
| | TMPKQKHYVE LKPYDKQKFE GGEALIKVLG NVANSGQCKK GLGKSNISIY | |
| | KVRTDVLGNQ HIIKNEGDKP KLDE | |
| SpCas9 | MDKKYSIGLD IGTNSVGWAV ITDEYKVPSK KFKVLGNTDR HSIKKNLIGA | (SEQ |
| inactive | LLFDSGETAE ATRLKRTARR RYTRRKNRIC YLQEIFSNEM AKVDDSFFHR | ID |
| AJN60011.1 | LEESELVEED KKHERHPIFG NIVDEVAYHE KYPTIYHLRK KLVDSTDKAD | NO: |
| GI: | LRLIYLALAH MIKFRGHFLI EGDLNPDNSD VDKLFIQLVQ TYNQLFEENP | 32) |
| 757015967 | INASGVDAKA ILSARLSKSR RLENLIAQLP GEKKNGLFGN LIALSLGLTP | |
| | NFKSNFDLAE DAKLQLSKDT YDDDLDNLLA QIGDQYADLF LAAKNLSDAI | |
| | LLSDILRVNT EITKAPLSAS MIKRYDEHHQ DLTLLKALVR QQLPEKYKEI | |
| | FFDQSKNGYA GYIDGGASQE EFYKFIKPIL EKMDGTEELL VKLNREDLLR | |
| | KQRTFDNGSI PHQIHLGELH AILRRQEDFY PFLKDNREKI EKILTFRIPY | |
| | YVGPLARGNS RFAWMTRKSE ETITPWNFEE VVDKGASAQS FIERMTNEDK | |
| | NLPNEKVLPK HSLLYEYFTV YNELTKVKYV TEGMRKPAFL SGEQKKAIVD | |
| | LLFKTNRKVT VKQLKEDYFK KIECFDSVEI SGVEDRFNAS LGTYHDLLKI | |
| | IKDKDELDNE ENEDILEDIV LTLTLFEDRE MIEERLKTYA HLFDDKVMKQ | |
| | LKRRRYTGWG RLSRKLINGI RDKQSGKTIL DFLKSDGFAN RNFMQLIHDD | |
| | SLTFKEDIQK AQVSGQGDSL HEHIANLAGS PAIKKGILQT VKVVDELVKV | |
| | MGRHKPENIV IAMARENQTT QKGQKNSRER MKRIEEGIKE LGSQILKEHP | |
| | VENTQLQNEK LYLYYLQNGR DMYVDQELDI NRLSDYDVDA IVPQSFLKDD | |
| | SIDAKVLTRS DKARGKSDNV PSEEVVKKMK NYWRQLLNAK LITQRKEDNL | |
| | TKAERGGLSE LDKAGFIKRQ LVETRQITKH VAQILDSRMN TKYDENDKLI | |
| | REVKVITLKS KLVSDFRKDF QFYKVREINN YHHAHAAYLN AVVGTALIKK | |
| | YPKLESEFVY GDYKVYDVRK MIAKSEQEIG KATAKYFFYS NIMNFFKTEI | |
| | TLANGEIRKR PLIETNGETG EIVWDKGRDF ATVRKVLSMP QVNIVKKTEV | |
| | QTGGFSKESI LPKRNSDKLI ARKKDWDPKK YGGFDSPTVA YSVLVVAKVE | |
| | KGKSKKLKSV KELLGITIME RSSFEKNPID FLEAKGYKEV KKDLIIKLPK | |
| | YSLFELENGR KRMLASAGEL QKGNELALPS KYVNFLYLAS HYEKLKGSPE | |
| | DNEQKQLFVE QHKHYLDEII EQISEFSKRV ILADANLDKV LSAYNKHRDK | |
| | PIREQAENII HLFTLINLGA PAAFKYFDTT IDRKRYTSTK EVLDATLIHQ | |
| | SITGLYETRI DLSQLGGD | |
| AJN60013.1 | MTQSERRFSC SIGIDMGAKY TGVFYALFDR EELPTNLNSK AMTLVMPETG | (SEQ |
| GI: | PRYVQAQRTA VRHRLRGQKR YTLARKLAFL VVDDMIKKQE KRLTDEEWKR | ID |
| 757015969 | GREALSGLLK RRGYSRPNAD GEDLTPLENV RADVFAAHPA FSTYFSEVRS | NO: |
| WP_005430658.1 | LAEQWEEFTA NISNVEKFLG DPNIPADKEF IEFAVAEGLI DKTEKKAYQS | 33) |
| Sutterella | ALSTLRANAN VLTGLRQMGH KPRSEYFKAI EADLKKDSRL AKINEAFGGA | |
| wadsworthensis | ERLARLLGNL SNLQLRAERW YFNAPDIMKD RGWEPDRFKK TLVRAFKFFH | |
| 3_1_45B | PAKDQNKQHL ELIKQIENSE DIIETLCTLD PNRTIPPYED QNNRRPPLDQ | |
| | TLLLSPEKLT RQYGEIWKTW SARLTSAEPT LAPAAEILER STDRKSRVAV | |
| | NGHEPLPTLA YQLSYALQRA FDRSKALDPY ALRALAAGSK SNKLTSARTA | |
| | LENCIGGQNV KTFLDCARRY YREADDAKVG LWFDNADGLL ERSDLHPPMK | |
| | KKILPLLVAN ILQTDETTGQ KFLDEIWRKQ IKGRETVASR CARIETVRKS | |
| | FGGGFNIAYN TAQYREVNKL PRNAQDKELL TIRDRVAETA DFIAANLGLS | |
| | DEQKRKFANP FSLAQFYTLI ETEVSGFSAT TLAVHLENAW RMTIKDAVIN | |
| | GETVRAAQCS RLPAETARPF DGLVRRLVDR QAWEIAKRVS TDIQSKVDES | |
| | NGIVDVSIFV EENKFEFSAS VADLKKNKRV KDKMLSEAEK LETRWLIKNE | |
| | RIKKASRGTC PYTGDRLAEG GEIDHILPRS LIKDARGIVE NAEPNLIYAS | |
| | SRGNQLKKNQ RYSLSDLKAN YRNEIFKTSN IAAITAEIED VVTKLQQTHR | |
| | LKFFDLLNEH EQDCVRHALF LDDGSEARDA VLELLATQRR TRVNGTQIWM | |
| | IKNLANKIRE ELQNWCKTTN NRLHFQAAAT NVSDAKNLRL KLAQNQPDFE | |
| | KPDIQPIASH SIDALCSFAV GSADAERDQN GFDYLDGKTV LGLYPQSCEV | |
| | IHLQAKPQEE KSHFDSVAIF KEGIYAEQFL PIFTLNEKIW IGYETLNAKG | |
| | ERCGAIEVSG KQPKELLEML APFFNKPVGD LSAHATYRIL KKPAYEFLAK | |
| | AALQPLSAEE KRLAALLDAL RYCTSRKSLM SLFMAANGKS LKKREDVLKP | |
| | KLFQLKVELK GEKSFKLNGS LTLPVKQDWL RICDSPELAD AFGKPCSADE | |
| | LTSKLARIWK RPVMRDLAHA PVRREFSLPA IDNPSGGFRI RRTNLFGNEL | |
| | YQVHAINAKK YRGFASAGSN VDWSKGILEN ELQHENLTEC GGRFITSADV | |
| | TPMSEWRKVV AEDNLSIWIA PGTEGRRYVR VETTFIQASH WFEQSVENWA | |
| | ITSPLSLPAS FKVDKPAEFQ KAVGTELSEL LGQPRSEIFI ENVGNAKHIR | |
| | FWYIVVSSNK KMNESYNNVS KS | |
| AJN60014.1 | MESSQILSPI GIDLGGKFTG VCLSHLEAFA ELPNHANTKY SVILIDHNNF | (SEQ |
| GI: | QLSQAQRRAT RHRVRNKKRN QFVKRVALQL FQHILSRDLN AKEETALCHY | ID |
| 757015970 | LNNRGYTYVD TDLDEYIKDE TTINLLKELL PSESEHNFID WFLQKMQSSE | NO: |
| WP_011212792.1 | FRKILVSKVE EKKDDKELKN AVKNIKNFIT GFEKNSVEGH RHRKVYFENI | 34) |
| Legionella | KSDITKDNQL DSIKKKIPSV CLSNLLGHLS NLQWKNLHRY LAKNPKQFDE | |
| pneumophila | QTFGNEFLRM LKNFRHLKGS QESLAVRNLI QQLEQSQDYI SILEKTPPEI | |
| str. Paris | TIPPYEARTN TGMEKDQSLL LNPEKLNNLY PNWRNLIPGI IDAHPFLEKD | |
| | LEHTKLRDRK RIISPSKQDE KRDSYILQRY LDLNKKIDKF KIKKQLSFLG | |
| | QGKQLPANLI ETQKEMETHF NSSLVSVLIQ IASAYNKERE DAAQGIWEDN | |
| | AFSLCELSNI NPPRKQKILP LLVGAILSED FINNKDKWAK FKIFWNTHKI | |
| | GRTSLKSKCK EIEEARKNSG NAFKIDYEEA LNHPEHSNNK ALIKIIQTIP | |
| | DIIQAIQSHL GHNDSQALIY HNPFSLSQLY TILETKRDGF HKNCVAVTCE | |
| | NYWRSQKTEI DPEISYASRL PADSVRPFDG VLARMMQRLA YEIAMAKWEQ | |
| | IKHIPDNSSL LIPIYLEQNR FEFEESFKKI KGSSSDKTLE QAIEKQNIQW | |
| | EEKFQRIINA SMNICPYKGA SIGGQGEIDH IYPRSLSKKH FGVIFNSEVN | |
| | LIYCSSQGNR EKKEEHYLLE HLSPLYLKHQ FGTDNVSDIK NFISQNVANI | |
| | KKYISFHLLT PEQQKAARHA LFLDYDDEAF KTITKFLMSQ QKARVNGTQK | |
| | FLGKQIMEFL STLADSKQLQ LEFSIKQITA EEVHDHRELL SKQEPKLVKS | |
| | RQQSFPSHAI DATLTMSIGL KEFPQFSQEL DNSWFINHLM PDEVHLNPVR | |
| | SKEKYNKPNI SSTPLFKDSL YAERFIPVWV KGETFAIGFS EKDLFEIKPS | |
| | NKEKLFTLLK TYSTKNPGES LQELQAKSKA KWLYFPINKT LALEFLHHYF | |
| | HKEIVTPDDT TVCHFINSLR YYTKKESITV KILKEPMPVL SVKFESSKKN | |
| | VLGSFKHTIA LPATKDWERL FNHPNFLALK ANPAPNPKEF NEFIRKYFLS | |
| | DNNPNSDIPN NGHNIKPQKH KAVRKVFSLP VIPGNAGTMM RIRRKDNKGQ | |
| | PLYQLQTIDD TPSMGIQINE DRLVKQEVLM DAYKIRNLST IDGINNSEGQ | |
| | AYATFDNWLT LPVSTFKPEI IKLEMKPHSK TRRYIRITQS LADFIKTIDE | |
| | ALMIKPSDSI DDPLNMPNEI VCKNKLFGNE LKPRDGKMKI VSTGKIVTYE | |
| | FESDSTPQWI QTLYVTQLKK QP | |
| AJN60015.1 | MKKEIKDYFL GLDVGTGSVG WAVTDTDYKL LKANRKDLWG MRCFETAETA | (SEQ |
| GI: | EVRRLHRGAR RRIERRKKRI KLLQELFSQE IAKTDEGFFQ RMKESPFYAE | ID |
| 757015971 | DKTILQENTL FNDKDFADKT YHKAYPTINH LIKAWIENKV KPDPRLLYLA | NO: |
| WP_002681289.1 | CHNIIKKRGH FLFEGDFDSE NQFDTSIQAL FEYLREDMEV DIDADSQKVK | 35) |
| Treponema | EILKDSSLKN SEKQSRLNKI LGLKPSDKQK KAITNLISGN KINFADLYDN | |
| denticola | PDLKDAEKNS ISFSKDDFDA LSDDLASILG DSFELLLKAK AVYNCSVLSK | |
| ATCC | VIGDEQYLSF AKVKIYEKHK TDLTKLKNVI KKHFPKDYKK VFGYNKNEKN | |
| 35405 | NNNYSGYVGV CKTKSKKLII NNSVNQEDFY KELKTILSAK SEIKEVNDIL | |
| | TEIETGTFLP KQISKSNAEI PYQLRKMELE KILSNAEKHF SFLKQKDEKG | |
| | LSHSEKIIML LTFKIPYYIG PINDNHKKFF PDRCWVVKKE KSPSGKTTPW | |
| | NFFDHIDKEK TAEAFITSRT NFCTYLVGES VLPKSSLLYS EYTVLNEINN | |
| | LQIIIDGKNI CDIKLKQKIY EDLFKKYKKI TQKQISTFIK HEGICNKTDE | |
| | VIILGIDKEC TSSLKSYIEL KNIFGKQVDE ISTKNMLEEI IRWATIYDEG | |
| | EGKTILKTKI KAEYGKYCSD EQIKKILNLK FSGWGRLSRK FLETVTSEMP | |
| | GFSEPVNIIT AMRETQNNLM ELLSSEFTFT ENIKKINSGF EDAEKQFSYD | |
| | GLVKPLFLSP SVKKMLWQTL KLVKEISHIT QAPPKKIFIE MAKGAELEPA | |
| | RTKTRLKILQ DLYNNCKNDA DAFSSEIKDL SGKIENEDNL RLRSDKLYLY | |
| | YTQLGKCMYC GKPIEIGHVF DTSNYDIDHI YPQSKIKDDS ISNRVLVCSS | |
| | CNKNKEDKYP LKSEIQSKQR GFWNFLQRNN FISLEKLNRL TRATPISDDE | |
| | TAKFIARQLV ETRQATKVAA KVLEKMFPET KIVYSKAETV SMFRNKFDIV | |
| | KCREINDFHH AHDAYLNIVV GNVYNTKFTN NPWNFIKEKR DNPKIADTYN | |
| | YYKVFDYDVK RNNITAWEKG KTIITVKDML KRNTPIYTRQ AACKKGELEN | |
| | QTIMKKGLGQ HPLKKEGPFS NISKYGGYNK VSAAYYTLIE YEEKGNKIRS | |
| | LETIPLYLVK DIQKDQDVLK SYLTDLLGKK EFKILVPKIK INSLLKINGF | |
| | PCHITGKIND SELLRPAVQF CCSNNEVLYF KKIIRFSEIR SQREKIGKTI | |
| | SPYEDLSFRS YIKENLWKKT KNDEIGEKEF YDLLQKKNLE IYDMLLTKHK | |
| | DTIYKKRPNS ATIDILVKGK EKFKSLIIEN QFEVILEILK LFSATRNVSD | |
| | LQHIGGSKYS GVAKIGNKIS SLDNCILIYQ SITGIFEKRI DLLKV | |
| AJN60016.1 | MTKEYYLGLD VGTNSVGWAV TDSQYNLCKF KKKDMWGIRL FESANTAKDR | (SEQ |
| GI: | RLQRGNRRRL ERKKQRIDLL QEIFSPEICK IDPTFFIRLN ESRLHLEDKS | ID |
| 757015972 | NDFKYPLFIE KDYSDIEYYK EFPTIFHLRK HLIESEEKQD IRLIYLALHN | NO: |
| EFE28295.1 | IIKTRGHFLI DGDLQSAKQL RPILDTELLS LQEEQNLSVS LSENQKDEYE | 36) |
| Filifactor | EILKNRSIAK SEKVKKLKNL FEISDELEKE EKKAQSAVIE NFCKFIVGNK | |
| alocis | GDVCKFLRVS KEELEIDSFS FSEGKYEDDI VKNLEEKVPE KVYLFEQMKA | |
| ATCC | MYDWNILVDI LETEEYISFA KVKQYEKHKT NLRLLRDIIL KYCTKDEYNR | |
| 35896 | MFNDEKEAGS YTAYVGKLKK NNKKYWIEKK RNPEEFYKSL GKLLDKIEPL | |
| | KEDLEVLTMM IEECKNHTLL PIQKNKDNGV IPHQVHEVEL KKILENAKKY | |
| | YSFLTETDKD GYSVVQKIES IFRFRIPYYV GPLSTRHQEK GSNVWMVRKP | |
| | GREDRIYPWN MEEIIDFEKS NENFITRMTN KCTYLIGEDV LPKHSLLYSK | |
| | YMVLNELNNV KVRGKKLPTS LKQKVFEDLF ENKSKVTGKN LLEYLQIQDK | |
| | DIQIDDLSGF DKDFKTSLKS YLDFKKQIFG EEIEKESIQN MIEDIIKWIT | |
| | IYGNDKEMLK RVIRANYSNQ LTEEQMKKIT GFQYSGWGNF SKMFLKGISG | |
| | SDVSTGETFD IITAMWETDN NLMQILSKKF TFMDNVEDEN SGKVGKIDKI | |
| | TYDSTVKEMF LSPENKRAVW QTIQVAEEIK KVMGCEPKKI FIEMARGGEK | |
| | VKKRTKSRKA QLLELYAACE EDCRELIKEI EDRDERDENS MKLFLYYTQF | |
| | GKCMYSGDDI DINELIRGNS KWDRDHIYPQ SKIKDDSIDN LVLVNKTYNA | |
| | KKSNELLSED IQKKMHSFWL SLLNKKLITK SKYDRLTRKG DFTDEELSGF | |
| | IARQLVETRQ STKAIADIFK QIYSSEVVYV KSSLVSDERK KPLNYLKSRR | |
| | VNDYHHAKDA YLNIVVGNVY NKKFTSNPIQ WMKKNRDTNY SLNKVFEHDV | |
| | VINGEVIWEK CTYHEDTNTY DGGTLDRIRK IVERDNILYT EYAYCEKGEL | |
| | FNATIQNKNG NSTVSLKKGL DVKKYGGYFS ANTSYFSLIE FEDKKGDRAR | |
| | HIIGVPIYIA NMLEHSPSAF LEYCEQKGYQ NVRILVEKIK KNSLLIINGY | |
| | PLRIRGENEV DTSFKRAIQL KLDQKNYELV RNIEKFLEKY VEKKGNYPID | |
| | ENRDHITHEK MNQLYEVLLS KMKKENKKGM ADPSDRIEKS KPKFIKLEDL | |
| | IDKINVINKM LNLLRCDNDT KADLSLIELP KNAGSFVVKK NTIGKSKIIL | |
| | VNQSVTGLYE NRREL | |
| AJN60017.1 | MGRKPYILSL DIGTGSVGYA CMDKGENVLK YHDKDALGVY LFDGALTAQE | (SEQ |
| GI: | RRQFRTSRRR KNRRIKRLGL LQELLAPLVQ NPNFYQFQRQ FAWKNDNMDE | ID |
| 757015973 | KNKSLSEVLS FLGYESKKYP TIYHLQEALL LKDEKFDPEL IYMALYHLVK | NO: |
| WP_014613259.1 | YRGHFLFDHL KIENLINNDN MHDFVELIET YENLNNIKLN LDYEKTKVIY | 37) |
| Staphylococcus | EILKDNEMTK NDRAKRVKNM EKKLEQFSIM LLGLKENEGK LENHADNAEE | |
| pseudintermedius | LKGANQSHTF ADNYEENLTP FLTVEQSEFI ERANKIYLSL TLQDILKGKK | |
| ED99 | SMAMSKVAAY DKERNELKQV KDIVYKADST RTQFKKIFVS SKKSLKQYDA | |
| | TPNDQTFSSL CLFDQYLIRP KKQYSLLIKE LKKIIPQDSE LYFEAENDTL | |
| | LKVLNTTDNA SIPMQINLYE AETILRNQQK YHAEITDEMI EKVLSLIQFR | |
| | IPYYVGPLVN DHTASKFGWM ERKSNESIKP WNFDEVVDRS KSATQFIRRM | |
| | TNKCSYLINE DVLPKNSLLY QEMEVLNELN ATQIRLQTDP KNRKYRMMPQ | |
| | IKLFAVEHIF KKYKTVSHSK FLEIMLNSNH RENFMNHGEK LSIFGTQDDK | |
| | KFASKLSSYQ DMTKIFGDIE GKRAQIEEII QWITIFEDKK ILVQKLKECY | |
| | PELTSKQINQ LKKLNYSGWG RLSEKLLTHA YQGHSIIELL RHSDENEMEI | |
| | LINDVYGFQN FIKEENQVQS NKIQHQDIAN LTTSPALKKG IWSTIKLVRE | |
| | LTSIFGEPEK IIMEFATEDQ QKGKKQKSRK QLWDDNIKKN KLKSVDEYKY | |
| | IIDVANKLNN EQLQQEKLWL YLSQNGKCMY SGQSIDLDAL LSPNATKHYE | |
| | VDHIFPRSFI KDDSIDNKVL VIKKMNQTKG DQVPLQFIQQ PYERIAYWKS | |
| | LNKAGLISDS KLHKLMKPEF TAMDKEGFIQ RQLVETRQIS VHVRDELKEE | |
| | YPNTKVIPMK AKMVSEFRKK FDIPKIRQMN DAHHAIDAYL NGVVYHGAQL | |
| | AYPNVDLFDF NFKWEKVREK WKALGEFNTK QKSRELFFFK KLEKMEVSQG | |
| | ERLISKIKLD MNHFKINYSR KLANIPQQFY NQTAVSPKTA ELKYESNKSN | |
| | EVVYKGLTPY QTYVVAIKSV NKKGKEKMEY QMIDHYVFDF YKFQNGNEKE | |
| | LALYLAQREN KDEVLDAQIV YSLNKGDLLY INNHPCYFVS RKEVINAKQF | |
| | ELTVEQQLSL YNVMNNKETN VEKLLIEYDF IAEKVINEYH HYLNSKLKEK | |
| | RVRTFFSESN QTHEDFIKAL DELFKVVTAS ATRSDKIGSR KNSMTHRAFL | |
| | GKGKDVKIAY TSISGLKTTK PKSLFKLAES RNEL | |
| AJN60018.1 | MTKIKDDYIV GLDIGTDSCG WVAMNSNNDI LKLQGKTAIG SRLFEGGKSA | (SEQ |
| GI: | AERRLFRTTH RRIKRRRWRL KLLEEFFDPY MAEVDPYFFA RLKESGLSPL | ID |
| 757015974 | DKRKTVSSIV FPTSAEDKKF YDDYPTIYHL RYKLMTEDEK FDLREVYLAI | NO: |
| WP_014567561.1 | HHIIKYRGNF LYNTSVKDFK ASKIDVKSSI EKLNELYENI GLDLNVEFNI | 38) |
| Lactobacillus | SNTAEIEKVL KDKQIFKRDK VKKIAELFAI KTDNKEQSKR IKDISKQVAN | |
| johnsonii | AVLGYKTRED TIALKEISKD ELSDWNFKLS DIDADSKFEA LMGNLDENEQ | |
| DPC 6026 | AILLTIKELF NEVTLNGIVE DGNTLSESMI NKYNDHRDDL KLLKEVIENH | |
| | IDRKKAKELA LAYDLYVNNR HGQLLQAKKK LGKIKPRSKE DFYKVVNKNL | |
| | DDSRASKEIK KKIELDSEMP KQRTNANGVI PYQLQQLELD KIIENQSKYY | |
| | PFLKEINPVS SHLKEAPYKL DELIRFRVPY YVGPLISPNE STKDIQTKKN | |
| | QNFAWMIRKE EGRITPWNED QKVDRIESAN KFIKRMTTKD TYLFGEDVLP | |
| | ANSLLYQKFT VLNELNNIRI NGKRISVDLK QEIYENLEKK HTTVTVKKLE | |
| | NYLKENHNLV KVEIKGLADE KKENSGLTTY NRFKNLNIFD NQIDDLKYRN | |
| | DFEKIIEWST IFEDKSIYKE KLRSIDWLNE KQINALSNIR LQGWGRLSKK | |
| | LLAQLHDHNG QTIIEQLWDS QNNFMQIVTQ ADFKDAIAKA NQNLLVATSV | |
| | EDILNNAYTS PANKKAIRQV IKVVDDIVKA ASGKVPKQIA IEFTRDADEN | |
| | PKRSQTRGSK LQKVYKDLST ELASKTIAEE LNEAIKDKKL VQDKYYLYFM | |
| | QLGRDAYTGE PINIDEIQKY DIDHILPQSF IKDDALDNRV LVSRAVNNGK | |
| | SDNVPVKLFG NEMAANLGMT IRKMWEEWKN IGLISKTKYN NLLTDPDHIN | |
| | KYKSAGFIRR QLVETSQIIK LVSTILQSRY PNTEIITVKA KYNHYLREKF | |
| | DLYKSREVND YHHAIDAYLS AICGNLLYQN YPNLRPFFVY GQYKKFSSDP | |
| | DKEKAIFNKT RKESFISQLL KNKSENSKEI AKKLKRAYQF KYMLVSRETE | |
| | TRDQEMFKMT VYPRFSHDTV KAPRNLIPKK MGMSPDIYGG YTNNSDAYMV | |
| | IVRIDKKKGT EYKILGIPTR ELVNLKKAEK EDHYKSYLKE ILTPRILYNK | |
| | NGKRDKKITS FEIVKSKIPY KQVIQDGDKK FMLGSSTYVY NAKQLTLSTE | |
| | SMKAITNNFD KDSDENDALI KAYDEILDKV DKYLPLFDIN KFREKLHSGR | |
| | EKFIKLSLED KKDTILKVLE GLHDNAVMTK IPTIGLSTPL GFMQFPNGVI | |
| | LSENAKLIYQ SPTGLFKKSV KISDL | |
| Mycoplasma | MNNSIKSKPE VTIGLDLGVG SVGWAIVDNE TNIIHHLGSR LFSQAKTAED | (SEQ |
| gallisepticum | RRSFRGVRRL IRRRKYKLKR FVNLIWKYNS YFGFKNKEDI LNNYQEQQKL | ID |
| str. F | HNTVLNLKSE ALNAKIDPKA LSWILHDYLK NRGHFYEDNR DENVYPTKEL | NO: |
| AJN60022.1 | AKYFDKYGYY KGIIDSKEDN DNKLEEELTK YKFSNKHWLE EVKKVLSNQT | 39) |
| GI: | GLPEKFKEEY ESLFSYVRNY SEGPGSINSV SPYGIYHLDE KEGKVVQKYN | |
| 757015978 | NIWDKTIGKC NIFPDEYRAP KNSPIAMIEN EINELSTIRS YSIYLTGWFI | |
| WP_014574789.1 | NQEFKKAYLN KLLDLLIKTN GEKPIDARQF KKLREETIAE SIGKETLKDV | |
| | ENEEKLEKED HKWKLKGLKL NINGKIQYND LSSLAKFVHK LKQHLKLDEL | |
| | LEDQYATLDK INFLQSLFVY LGKHLRYSNR VDSANLKEFS DSNKLFERIL | |
| | QKQKDGLFKL FEQTDKDDEK ILAQTHSLST KAMLLAITRM TNLDNDEDNQ | |
| | KNNDKGWNFE AIKNFDQKFI DITKKNNNLS LKQNKRYLDD RFINDAILSP | |
| | GVKRILREAT KVENAILKQF SEEYDVTKVV IELARELSEE KELENTKNYK | |
| | KLIKKNGDKI SEGLKALGIS EDEIKDILKS PTKSYKFLLW LQQDHIDPYS | |
| | LKEIAFDDIF TKTEKFEIDH IIPYSISFDD SSSNKLLVLA ESNQAKSNQT | |
| | PYEFISSGNA GIKWEDYEAY CRKFKDGDSS LLDSTQRSKK FAKMMKTDTS | |
| | SKYDIGFLAR NLNDTRYATI VFRDALEDYA NNHLVEDKPM FKVVCINGSV | |
| | TSFLRKNFDD SSYAKKDRDK NIHHAVDASI ISIFSNETKT LFNQLTQFAD | |
| | YKLFKNTDGS WKKIDPKTGV VTEVTDENWK QIRVRNQVSE IAKVIEKYIQ | |
| | DSNIERKARY SRKIENKTNI SLFNDTVYSA KKVGYEDQIK RKNLKTLDIH | |
| | ESAKENKNSK VKRQFVYRKL VNVSLLNNDK LADLFAEKED ILMYRANPWV | |
| | INLAEQIFNE YTENKKIKSQ NVFEKYMLDL TKEFPEKFSE FLVKSMLRNK | |
| | TAIIYDDKKN IVHRIKRLKM LSSELKENKL SNVIIRSKNQ SGTKLSYQDT | |
| | INSLALMIMR SIDPTAKKQY IRVPLNTLNL HLGDHDFDLH NMDAYLKKPK | |
| | FVKYLKANEI GDEYKPWRVL TSGTLLIHKK DKKLMYISSF QNLNDVIEIK | |
| | NLIETEYKEN DDSDSKKKKK ANRFLMTLST ILNDYILLDA KDNFDILGLS | |
| | KNRIDEILNS KLGLDKIVK | |
| AJN60023.1 | MRILGFDIGI NSIGWAFVEN DELKDCGVRI FTKAENPKNK ESLALPRRNA | (SEQ |
| GI: | RSSRRRLKRR KARLIAIKRI LAKELKLNYK DYVAADGELP KAYEGSLASV | ID |
| 757015979 | YELRYKALTQ NLETKDLARV ILHIAKHRGY MNKNEKKSND AKKGKILSAL | NO: |
| | KNNALKLENY QSVGEYFYKE FFQKYKKNTK NFIKIRNTKD NYNNCVLSSD | 30) |
| | LEKELKLILE KQKEFGYNYS EDFINEILKV AFFQRPLKDF SHLVGACTFF | |
| | EEEKRACKNS YSAWEFVALT KIINEIKSLE KISGEIVPTQ TINEVLNLIL | |
| | DKGSITYKKF RSCINLHESI SFKSLKYDKE NAENAKLIDF RKLVEFKKAL | |
| | GVHSLSRQEL DQISTHITLI KDNVKLKTVL EKYNLSNEQI NNLLEIEEND | |
| | YINLSFKALG MILPLMREGK RYDEACEIAN LKPKTVDEKK DFLPAFCDSI | |
| | FAHELSNPVV NRAISEYRKV LNALLKKYGK VHKIHLELAR DVGLSKKARE | |
| | KIEKEQKENQ AVNAWALKEC ENIGLKASAK NILKLKLWKE QKEICIYSGN | |
| | KISIEHLKDE KALEVDHIYP YSRSFDDSFI NKVLVFTKEN QEKLNKTPFE | |
| | AFGKNIEKWS KIQTLAQNLP YKKKNKILDE NFKDKQQEDF ISRNLNDTRY | |
| | IATLIAKYTK EYLNFLLLSE NENANLKSGE KGSKIHVQTI SGMLTSVLRH | |
| | TWGFDKKDRN NHLHHALDAI IVAYSINSII KAFSDFRKNQ ELLKARFYAK | |
| | ELTSDNYKHQ VKFFEPFKSF REKILSKIDE IFVSKPPRKR ARRALHKDTF | |
| | HSENKIIDKC SYNSKEGLQI ALSCGRVRKI GTKYVENDTI VRVDIFKKQN | |
| | KFYAIPIYAM DEALGILPNK IVITGKDKNN NPKQWQTIDE SYEFCFSLYK | |
| | NDLILLQKKN MQEPEFAYYN DFSISTSSIC VEKHDNKFEN LTSNQKLLES | |
| | NAKEGSVKVE SLGIQNLKVF EKYIITPLGD KIKADFQPRE NISLKTSKKY | |
| | GLR | |
| AJN60025.1 | MSDLVLGLDI GIGSVGVGIL NKVTGEIIHK NSRIFPAAQA ENNLVRRTNR | (SEQ |
| GI: | QGRRLARRKK HRRVRLNRLF EESGLITDFT KISININPYQ LRVKGLTDEL | ID |
| 757015981 | SNEELFIALK NMVKHRGISY LDDASDDGNS SVGDYAQIVK ENSKQLETKT | NO: |
| | PGQIQLERYQ TYGQLRGDFT VEKDGKKHRL INVFPTSAYR SEALRILQTQ | 41) |
| | QEFNPQITDE FINRYLEILT GKRKYYHGPG NEKSRTDYGR YRTSGETLDN | |
| | IFGILIGKCT FYPDEFRAAK ASYTAQEFNL LNDLNNLTVP TETKKLSKEQ | |
| | KNQIINYVKN EKAMGPAKLF KYIAKLLSCD VADIKGYRID KSGKAEIHTF | |
| | EAYRKMKTLE TLDIEQMDRE TLDKLAYVLT LNTEREGIQE ALEHEFADGS | |
| | FSQKQVDELV QFRKANSSIF GKGWHNFSVK LMMELIPELY ETSEEQMTIL | |
| | TRLGKQKTTS SSNKTKYIDE KLLTEEIYNP VVAKSVRQAI KIVNAAIKEY | |
| | GDFDNIVIEM ARETNEDDEK KAIQKIQKAN KDEKDAAMLK AANQYNGKAE | |
| | LPHSVFHGHK QLATKIRLWH QQGERCLYTG KTISIHDLIN NSNQFEVDHI | |
| | LPLSITFDDS LANKVLVYAT ANQEKGQRTP YQALDSMDDA WSFRELKAFV | |
| | RESKTLSNKK KEYLLTEEDI SKFDVRKKFI ERNLVDTRYA SRVVLNALQE | |
| | HFRAHKIDTK VSVVRGQFTS QLRRHWGIEK TRDTYHHHAV DALIIAASSQ | |
| | LNLWKKQKNT LVSYSEDQLL DIETGELISD DEYKESVFKA PYQHFVDTLK | |
| | SKEFEDSILF SYQVDSKFNR KISDATIYAT RQAKVGKDKA DETYVLGKIK | |
| | DIYTQDGYDA FMKIYKKDKS KFLMYRHDPQ TFEKVIEPIL ENYPNKQINE | |
| | KGKEVPCNPF LKYKEEHGYI RKYSKKGNGP EIKSLKYYDS KLGNHIDITP | |
| | KDSNNKVVLQ SVSPWRADVY FNKTTGKYEI LGLKYADLQF EKGTGTYKIS | |
| | QEKYNDIKKK EGVDSDSEFK FTLYKNDLLL VKDTETKEQQ LFRFLSRTMP | |
| | KQKHYVELKP YDKQKFEGGE ALIKVLGNVA NSGQCKKGLG KSNISIYKVR | |
| | TDVLGNQHII KNEGDKPKLM | |
| WP_002664048.1 | MKHILGLDLG TNSIGWALIE RNIEEKYGKI IGMGSRIVPM GAELSKFEQG | (SEQ |
| Bergeyella | QAQTKNADRR TNRGARRLNK RYKQRRNKLI YILQKLDMLP SQIKLKEDES | ID |
| zoohelcum | DPNKIDKITI LPISKKQEQL TAFDLVSLRV KALTEKVGLE DLGKIIYKYN | NO |
| ATCC | QLRGYAGGSL EPEKEDIFDE EQSKDKKNKS FIAFSKIVFL GEPQEEIFKN | 42) |
| 43767 | KKLNRRAIIV ETEEGNFEGS TFLENIKVGD SLELLINISA SKSGDTITIK | |
| | LPNKTNWRKK MENIENQLKE KSKEMGREFY ISEFLLELLK ENRWAKIRNN | |
| | TILRARYESE FEAIWNEQVK HYPFLENLDK KTLIEIVSFI FPGEKESQKK | |
| | YRELGLEKGL KYIIKNQVVF YQRELKDQSH LISDCRYEPN EKAIAKSHPV | |
| | FQEYKVWEQI NKLIVNTKIE AGTNRKGEKK YKYIDRPIPT ALKEWIFEEL | |
| | QNKKEITFSA IFKKLKAEFD LREGIDFLNG MSPKDKLKGN ETKLQLQKSL | |
| | GELWDVLGLD SINRQIELWN ILYNEKGNEY DLTSDRISKV LEFINKYGNN | |
| | IVDDNAEETA IRISKIKFAR AYSSLSLKAV ERILPLVRAG KYFNNDESQQ | |
| | LQSKILKLLN ENVEDPFAKA AQTYLDNNQS VLSEGGVGNS IATILVYDKH | |
| | TAKEYSHDEL YKSYKEINLL KQGDLRNPLV EQIINEALVL IRDIWKNYGI | |
| | KPNEIRVELA RDLKNSAKER ATIHKRNKDN QTINNKIKET LVKNKKELSL | |
| | ANIEKVKLWE AQRHLSPYTG QPIPLSDLED KEKYDVDHII PISRYFDDSF | |
| | TNKVISEKSV NQEKANRTAM EYFEVGSLKY SIFTKEQFIA HVNEYFSGVK | |
| | RKNLLATSIP EDPVQRQIKD TQYIAIRVKE ELNKIVGNEN VKTTTGSITD | |
| | YLRNHWGLTD KFKLLLKERY EALLESEKFL EAEYDNYKKD FDSRKKEYEE | |
| | KEVLFEEQEL TREEFIKEYK ENYIRYKKNK LIIKGWSKRI DHRHHAIDAL | |
| | IVACTEPAHI KRLNDLNKVL QDWLVEHKSE FMPNFEGSNS ELLEEILSLP | |
| | ENERTEIFTQ IEKFRAIEMP WKGFPEQVEQ KLKEIIISHK PKDKLLLQYN | |
| | KAGDRQIKLR GQLHEGTLYG ISQGKEAYRI PLTKFGGSKF ATEKNIQKIV | |
| | SPFLSGFIAN HLKEYNNKKE EAFSAEGIMD LNNKLAQYRN EKGELKPHTP | |
| | ISTVKIYYKD PSKNKKKKDE EDLSLQKLDR EKAFNEKLYV KTGDNYLFAV | |
| | LEGEIKTKKT SQIKRLYDII SFFDATNFLK EEFRNAPDKK TFDKDLLFRQ | |
| | YFEERNKAKL LFTLKQGDFV YLPNENEEVI LDKESPLYNQ YWGDLKERGK | |
| | NIYVVQKFSK KQIYFIKHTI ADIIKKDVEF GSQNCYETVE GRSIKENCFK | |
| | LEIDRLGNIV KVIKR | |
| CBK78998.1 | MKQEYFLGLD MGTGSLGWAV TDSTYQVMRK HGKALWGTRL FESASTAEER | (SEQ |
| Coprococcus | RMFRTARRRL DRRNWRIQVL QEIFSEEISK VDPGFFLRMK ESKYYPEDKR | ID |
| catus | DAEGNCPELP YALFVDDNYT DKNYHKDYPT IYHLRKMLME TTEIPDIRLV | NO: |
| GD/7 | YLVLHHMMKH RGHFLLSGDI SQIKEFKSTF EQLIQNIQDE ELEWHISLDD | 43) |
| | AAIQFVEHVL KDRNLTRSTK KSRLIKQLNA KSACEKAILN LLSGGTVKLS | |
| | DIFNNKELDE SERPKVSFAD SGYDDYIGIV EAELAEQYYI IASAKAVYDW | |
| | SVLVEILGNS VSISEAKIKV YQKHQADLKT LKKIVRQYMT KEDYKRVFVD | |
| | TEEKLNNYSA YIGMTKKNGK KVDLKSKQCT QADFYDFLKK NVIKVIDHKE | |
| | ITQEIESEIE KENFLPKQVT KDNGVIPYQV HDYELKKILD NLGTRMPFIK | |
| | ENAEKIQQLF EFRIPYYVGP LNRVDDGKDG KFTWSVRKSD ARIYPWNFTE | |
| | VIDVEASAEK FIRRMTNKCT YLVGEDVLPK DSLVYSKFMV LNELNNLRLN | |
| | GEKISVELKQ RIYEELFCKY RKVTRKKLER YLVIEGIAKK GVEITGIDGD | |
| | FKASLTAYHD FKERLTDVQL SQRAKEAIVL NVVLFGDDKK LLKQRLSKMY | |
| | PNLTTGQLKG ICSLSYQGWG RLSKTFLEEI TVPAPGTGEV WNIMTALWQT | |
| | NDNLMQLLSR NYGFTNEVEE FNTLKKETDL SYKTVDELYV SPAVKRQIWQ | |
| | TLKVVKEIQK VMGNAPKRVF VEMAREKQEG KRSDSRKKQL VELYRACKNE | |
| | ERDWITELNA QSDQQLRSDK LFLYYIQKGR CMYSGETIQL DELWDNTKYD | |
| | IDHIYPQSKT MDDSLNNRVL VKKNYNAIKS DTYPLSLDIQ KKMMSFWKML | |
| | QQQGFITKEK YVRLVRSDEL SADELAGFIE RQIVETRQST KAVATILKEA | |
| | LPDTEIVYVK AGNVSNFRQT YELLKVREMN DLHHAKDAYL NIVVGNAYFV | |
| | KFTKNAAWFI RNNPGRSYNL KRMFEFDIER SGEIAWKAGN KGSIVTVKKV | |
| | MQKNNILVTR KAYEVKGGLF DQQIMKKGKG QVPIKGNDER LADIEKYGGY | |
| | NKAAGTYFML VKSLDKKGKE IRTIEFVPLY LKNQIEINHE SAIQYLAQER | |
| | GLNSPEILLS KIKIDTLFKV DGFKMWLSGR TGNQLIFKGA NQLILSHQEA | |
| | AILKGVVKYV NRKNENKDAK LSERDGMTEE KLLQLYDTFL DKLSNTVYSI | |
| | RLSAQIKTLT EKRAKFIGLS NEDQCIVLNE ILHMFQCQSG SANLKLIGGP | |
| | GSAGILVMNN NITACKQISV INQSPTGIYE KEIDLIKL | |
| WP_002235162.1 | MAAFKPNPIN YILGLDIGIA SVGWAMVEID EDENPICLID LGVRVFERAE | (SEQ |
| Neisseria | VPKTGDSLAM ARRLARSVRR LTRRRAHRLL RARRLLKREG VLQAADFDEN | ID |
| meningitidis | GLIKSLPNTP WQLRAAALDR KLTPLEWSAV LLHLIKHRGY LSQRKNEGET | NO: |
| Z2491 | ADKELGALLK GVADNAHALQ TGDERTPAEL ALNKFEKESG HIRNQRGDYS | 44) |
| | HTFSRKDLQA ELILLFEKQK EFGNPHVSGG LKEGIETLLM TQRPALSGDA | |
| | VQKMLGHCTF EPAEPKAAKN TYTAERFIWL TKLNNLRILE QGSERPLTDT | |
| | ERATLMDEPY RKSKLTYAQA RKLLGLEDTA FFKGLRYGKD NAEASTLMEM | |
| | KAYHAISRAL EKEGLKDKKS PLNLSPELQD EIGTAFSLFK TDEDITGRLK | |
| | DRIQPEILEA LLKHISFDKF VQISLKALRR IVPLMEQGKR YDEACAEIYG | |
| | DHYGKKNTEE KIYLPPIPAD EIRNPVVLRA LSQARKVING VVRRYGSPAR | |
| | IHIETAREVG KSFKDRKEIE KRQEENRKDR EKAAAKFREY FPNFVGEPKS | |
| | KDILKLRLYE QQHGKCLYSG KEINLGRLNE KGYVEIDHAL PFSRTWDDSF | |
| | NNKVLVLGSE NQNKGNQTPY EYFNGKDNSR EWQEFKARVE TSRFPRSKKQ | |
| | RILLQKFDED GFKERNLNDT RYVNRFLCQF VADRMRLTGK GKKRVFASNG | |
| | QITNLLRGFW GLRKVRAEND RHHALDAVVV ACSTVAMQQK ITRFVRYKEM | |
| | NAFDGKTIDK ETGEVLHQKT HFPQPWEFFA QEVMIRVFGK PDGKPEFEEA | |
| | DTPEKLRTLL AEKLSSRPEA VHEYVTPLFV SRAPNRKMSG QGHMETVKSA | |
| | KRLDEGVSVL RVPLTQLKLK DLEKMVNRER EPKLYEALKA RLEAHKDDPA | |
| | KAFAEPFYKY DKAGNRTQQV KAVRVEQVQK TGVWVRNHNG IADNATMVRV | |
| | DVFEKGDKYY LVPIYSWQVA KGILPDRAVV QGKDEEDWQL IDDSENFKES | |
| | LHPNDLVEVI TKKARMFGYF ASCHRGTGNI NIRIHDLDHK IGKNGILEGI | |
| | GVKTALSFQK YQIDELGKEI RPCRLKKRPP VR | |
| WP_012414420.1 | MQKNINTKQN HIYIKQAQKI KEKLGDKPYR IGLDLGVGSI GFAIVSMEEN | (SEQ |
| Elusimicrobium | DGNVLLPKEI IMVGSRIFKA SAGAADRKLS RGQRNNHRHT RERMRYLWKV | ID |
| minutum | LAEQKLALPV PADLDRKENS SEGETSAKRF LGDVLQKDIY ELRVKSLDER | NO: |
| Pei191 | LSLQELGYVL YHIAGHRGSS AIRTFENDSE EAQKENTENK KIAGNIKRLM | 45) |
| | AKKNYRTYGE YLYKEFFENK EKHKREKISN AANNHKFSPT RDLVIKEAEA | |
| | ILKKQAGKDG FHKELTEEYI EKLTKAIGYE SEKLIPESGF CPYLKDEKRL | |
| | PASHKLNEER RLWETLNNAR YSDPIVDIVT GEITGYYEKQ FTKEQKQKLF | |
| | DYLLTGSELT PAQTKKLLGL KNTNFEDIIL QGRDKKAQKI KGYKLIKLES | |
| | MPFWARLSEA QQDSFLYDWN SCPDEKLLTE KLSNEYHLTE EEIDNAFNEI | |
| | VLSSSYAPLG KSAMLIILEK IKNDLSYTEA VEEALKEGKL TKEKQAIKDR | |
| | LPYYGAVLQE STQKIIAKGF SPQFKDKGYK TPHTNKYELE YGRIANPVVH | |
| | QTLNELRKLV NEIIDILGKK PCEIGLETAR ELKKSAEDRS KLSREQNDNE | |
| | SNRNRIYEIY IRPQQQVIIT RRENPRNYIL KFELLEEQKS QCPFCGGQIS | |
| | PNDIINNQAD IEHLFPIAES EDNGRNNLVI SHSACNADKA KRSPWAAFAS | |
| | AAKDSKYDYN RILSNVKENI PHKAWRENQG AFEKFIENKP MAARFKTDNS | |
| | YISKVAHKYL ACLFEKPNII CVKGSLTAQL RMAWGLQGLM IPFAKQLITE | |
| | KESESENKDV NSNKKIRLDN RHHALDAIVI AYASRGYGNL LNKMAGKDYK | |
| | INYSERNWLS KILLPPNNIV WENIDADLES FESSVKTALK NAFISVKHDH | |
| | SDNGELVKGT MYKIFYSERG YTLTTYKKLS ALKLTDPQKK KTPKDFLETA | |
| | LLKFKGRESE MKNEKIKSAI ENNKRLEDVI QDNLEKAKKL LEEENEKSKA | |
| | EGKKEKNIND ASIYQKAISL SGDKYVQLSK KEPGKFFAIS KPTPTTTGYG | |
| | YDTGDSLCVD LYYDNKGKLC GEIIRKIDAQ QKNPLKYKEQ GFTLFERIYG | |
| | GDILEVDEDI HSDKNSERNN TGSAPENRVF IKVGIFTEIT NNNIQIWFGN | |
| | IIKSTGGQDD SFTINSMQQY NPRKLILSSC GFIKYRSPIL KNKEG | |
| WP_009105777.1 | MIMKLEKWRL GLDLGTNSIG WSVFSLDKDN SVQDLIDMGV RIFSDGRDPK | (SEQ |
| Treponema | TKEPLAVARR TARSQRKLIY RRKLRRKQVF KFLQEQGLFP KTKEECMTLK | ID |
| sp. JC4 | SLNPYELRIK ALDEKLEPYE LGRALFNLAV RRGFKSNRKD GSREEVSEKK | NO: |
| | SPDEIKTQAD MQTHLEKAIK ENGCRTITEF LYKNQGENGG IRFAPGRMTY | 46) |
| | YPTRKMYEEE FNLIRSKQEK YYPQVDWDDI YKAIFYQRPL KPQQRGYCIY | |
| | ENDKERTFKA MPCSQKLRIL QDIGNLAYYE GGSKKRVELN DNQDKVLYEL | |
| | LNSKDKVTED QMRKALCLAD SNSFNLEENR DFLIGNPTAV KMRSKNRFGK | |
| | LWDEIPLEEQ DLIIETIITA DEDDAVYEVI KKYDLTQEQR DFIVKNTILQ | |
| | SGTSMLCKEV SEKLVKRLEE IADLKYHEAV ESLGYKFADQ TVEKYDLLPY | |
| | YGKVLPGSTM EIDLSAPETN PEKHYGKISN PTVHVALNQT RVVVNALIKE | |
| | YGKPSQIAIE LSRDLKNNVE KKAEIARKQN QRAKENIAIN DTISALYHTA | |
| | FPGKSFYPNR NDRMKYRLWS ELGLGNKCIY CGKGISGAEL FTKEIEIEHI | |
| | LPFSRTLLDA ESNLTVAHSS CNAFKAERSP FEAFGINPSG YSWQEIIQRA | |
| | NQLKNTSKKN KFSPNAMDSF EKDSSFIARQ LSDNQYIAKA ALRYLKCLVE | |
| | NPSDVWTTNG SMTKLLRDKW EMDSILCRKF TEKEVALLGL KPEQIGNYKK | |
| | NRFDHRHHAI DAVVIGLTDR SMVQKLATKN SHKGNRIEIP EFPILRSDLI | |
| | EKVKNIVVSF KPDHGAEGKL SKETLLGKIK LHGKETFVCR ENIVSLSEKN | |
| | LDDIVDEKIK SKVKDYVAKH KGQKIEAVLS DESKENGIKK VRCVNRVQTP | |
| | IEITSGKISR YLSPEDYFAA VIWEIPGEKK TFKAQYIRRN EVEKNSKGLN | |
| | VVKPAVLENG KPHPAAKQVC LLHKDDYLEF SDKGKMYFCR IAGYAATNNK | |
| | LDIRPVYAVS YCADWINSTN ETMLTGYWKP TPTQNWVSVN VLEDKQKARL | |
| | VTVSPIGRVF RK | |
| WP_002460848.1 | MNQKFILGLD IGITSVGYGL IDYETKNIID AGVRLFPEAN VENNEGRRSK | (SEQ |
| Staphylococcus | RGSRRLKRRR IHRLERVKKL LEDYNLLDQS QIPQSTNPYA IRVKGLSEAL | ID |
| lugdunensis | SKDELVIALL HIAKRRGIHK IDVIDSNDDV GNELSTKEQL NKNSKLLKDK | NO: |
| M23590 | FVCQIQLERM NEGQVRGEKN RFKTADIIKE IIQLLNVQKN FHQLDENFIN | 47) |
| | KYIELVEMRR EYFEGPGKGS PYGWEGDPKA WYETLMGHCT YFPDELRSVK | |
| | YAYSADLENA LNDLNNLVIQ RDGLSKLEYH EKYHIIENVF KQKKKPTLKQ | |
| | IANEINVNPE DIKGYRITKS GKPQFTEFKL YHDLKSVLFD QSILENEDVL | |
| | DQIAEILTIY QDKDSIKSKL TELDILLNEE DKENIAQLTG YTGTHRLSLK | |
| | CIRLVLEEQW YSSRNQMEIF THLNIKPKKI NLTAANKIPK AMIDEFILSP | |
| | VVKRTFGQAI NLINKIIEKY GVPEDIIIEL ARENNSKDKQ KFINEMQKKN | |
| | ENTRKRINEI IGKYGNQNAK RLVEKIRLHD EQEGKCLYSL ESIPLEDLLN | |
| | NPNHYEVDHI IPRSVSFDNS YHNKVLVKQS ENSKKSNLTP YQYFNSGKSK | |
| | LSYNQFKQHI LNLSKSQDRI SKKKKEYLLE ERDINKFEVQ KEFINRNLVD | |
| | TRYATRELTN YLKAYFSANN MNVKVKTING SFTDYLRKVW KFKKERNHGY | |
| | KHHAEDALII ANADELFKEN KKLKAVNSVL EKPEIESKQL DIQVDSEDNY | |
| | SEMFIIPKQV QDIKDERNFK YSHRVDKKPN RQLINDTLYS TRKKDNSTYI | |
| | VQTIKDIYAK DNTTLKKQFD KSPEKFLMYQ HDPRTFEKLE VIMKQYANEK | |
| | NPLAKYHEET GEYLTKYSKK NNGPIVKSLK YIGNKLGSHL DVTHQFKSST | |
| | KKLVKLSIKP YRFDVYLTDK GYKFITISYL DVLKKDNYYY IPEQKYDKLK | |
| | LGKAIDKNAK FIASFYKNDL IKLDGEIYKI IGVNSDTRNM IELDLPDIRY | |
| | KEYCELNNIK GEPRIKKTIG KKVNSIEKLT TDVLGNVFTN TQYTKPQLLE | |
| | KRGN | |
| WP_011681470.1 | MTKPYSIGLD IGTNSVGWAV TTDNYKVPSK KMKVLGNTSK KYIKKNLLGV | (SEQ |
| Streptococcus | LLFDSGITAE GRRLKRTARR RYTRRRNRIL YLQEIFSTEM ATLDDAFFQR | ID |
| thermophilus | LDDSFLVPDD KRDSKYPIFG NLVEEKAYHD EFPTIYHLRK YLADSTKKAD | NO: |
| LMD-9 | LRLVYLALAH MIKYRGHFLI EGEFNSKNND IQKNFQDELD TYNAIFESDL | 48) |
| | SLENSKQLEE IVKDKISKLE KKDRILKLFP GEKNSGIFSE FLKLIVGNQA | |
| | DERKCFNLDE KASLHESKES YDEDLETLLG YIGDDYSDVF LKAKKLYDAI | |
| | LLSGFLTVTD NETEAPLSSA MIKRYNEHKE DLALLKEYIR NISLKTYNEV | |
| | FKDDTKNGYA GYIDGKTNQE DFYVYLKKLL AEFEGADYFL EKIDREDFLR | |
| | KQRTFDNGSI PYQIHLQEMR AILDKQAKFY PFLAKNKERI EKILTFRIPY | |
| | YVGPLARGNS DFAWSIRKRN EKITPWNFED VIDKESSAEA FINRMTSFDL | |
| | YLPEEKVLPK HSLLYETFNV YNELTKVRFI AESMRDYQFL DSKQKKDIVR | |
| | LYFKDKRKVT DKDIIEYLHA IYGYDGIELK GIEKQFNSSL STYHDLLNII | |
| | NDKEFLDDSS NEAIIEEIIH TLTIFEDREM IKQRLSKFEN IFDKSVLKKL | |
| | SRRHYTGWGK LSAKLINGIR DEKSGNTILD YLIDDGISNR NFMQLIHDDA | |
| | LSFKKKIQKA QIIGDEDKGN IKEVVKSLPG SPAIKKGILQ SIKIVDELVK | |
| | VMGGRKPESI VVEMARENQY TNQGKSNSQQ RLKRLEKSLK ELGSKILKEN | |
| | IPAKLSKIDN NALQNDRLYL YYLQNGKDMY TGDDLDIDRL SNYDIDHIIP | |
| | QAFLKDNSID NKVLVSSASN RGKSDDVPSL EVVKKRKTFW YQLLKSKLIS | |
| | QRKFDNLTKA ERGGLSPEDK AGFIQRQLVE TRQITKHVAR LLDEKFNNKK | |
| | DENNRAVRTV KIITLKSTLV SQFRKDFELY KVREINDFHH AHDAYLNAVV | |
| | ASALLKKYPK LEPEFVYGDY PKYNSFRERK SATEKVYFYS NIMNIFKKSI | |
| | SLADGRVIER PLIEVNEETG ESVWNKESDL ATVRRVLSYP QVNVVKKVEE | |
| | QNHGLDRGKP KGLFNANLSS KPKPNSNENL VGAKEYLDPK KYGGYAGISN | |
| | SFTVLVKGTI EKGAKKKITN VLEFQGISIL DRINYRKDKL NELLEKGYKD | |
| | IELIIELPKY SLFELSDGSR RMLASILSTN NKRGEIHKGN QIFLSQKFVK | |
| | LLYHAKRISN TINENHRKYV ENHKKEFEEL FYYILEFNEN YVGAKKNGKL | |
| | LNSAFQSWQN HSIDELCSSF IGPTGSERKG LFELTSRGSA ADFEFLGVKI | |
| | PRYRDYTPSS LLKDATLIHQ SVTGLYETRI DLAKLGEG | |
| WP_009293010.1 | MKRILGLDLG TNSIGWALVN EAENKDERSS IVKLGVRVNP LTVDELTNFE | (SEQ |
| Bacteroides | KGKSITTNAD RTLKRGMRRN LQRYKLRRET LTEVLKEHKL ITEDTILSEN | ID |
| fragilis | GNRTTFETYR LRAKAVTEEI SLEEFARVLL MINKKRGYKS SRKAKGVEEG | NO: |
| NCTC 9343 | TLIDGMDIAR ELYNNNLTPG ELCLQLLDAG KKFLPDFYRS DLQNELDRIW | 49) |
| Cas9 | EKQKEYYPEI LTDVLKEELR GKKRDAVWAI CAKYFVWKEN YTEWNKEKGK | |
| | TEQQEREHKL EGIYSKRKRD EAKRENLQWR VNGLKEKLSL EQLVIVFQEM | |
| | NTQINNSSGY LGAISDRSKE LYFNKQTVGQ YQMEMLDKNP NASLRNMVFY | |
| | RQDYLDEFNM LWEKQAVYHK ELTEELKKEI RDIIIFYQRR LKSQKGLIGF | |
| | CEFESRQIEV DIDGKKKIKT VGNRVISRSS PLFQEFKIWQ ILNNIEVTVV | |
| | GKKRKRRKLK ENYSALFEEL NDAEQLELNG SRRLCQEEKE LLAQELFIRD | |
| | KMTKSEVLKL LFDNPQELDL NEKTIDGNKT GYALFQAYSK MIEMSGHEPV | |
| | DFKKPVEKVV EYIKAVEDLL NWNTDILGEN SNEELDNQPY YKLWHLLYSE | |
| | EGDNTPTGNG RLIQKMTELY GFEKEYATIL ANVSFQDDYG SLSAKAIHKI | |
| | LPHLKEGNRY DVACVYAGYR HSESSLTREE IANKVLKDRL MLLPKNSLHN | |
| | PVVEKILNQM VNVINVIIDI YGKPDEIRVE LARELKKNAK EREELTKSIA | |
| | QTTKAHEEYK TLLQTEFGLT NVSRTDILRY KLYKELESCG YKTLYSNTYI | |
| | SREKLFSKEF DIEHIIPQAR LEDDSESNKT LEARSVNIEK GNKTAYDFVK | |
| | EKFGESGADN SLEHYLNNIE DLFKSGKISK TKYNKLKMAE QDIPDGFIER | |
| | DLRNTQYIAK KALSMLNEIS HRVVATSGSV TDKLREDWQL IDVMKELNWE | |
| | KYKALGLVEY FEDRDGRQIG RIKDWTKRND HRHHAMDALT VAFTKDVFIQ | |
| | YFNNKNASLD PNANEHAIKN KYFQNGRAIA PMPLREFRAE AKKHLENTLI | |
| | SIKAKNKVIT GNINKTRKKG GVNKNMQQTP RGQLHLETIY GSGKQYLTKE | |
| | EKVNASFDMR KIGTVSKSAY RDALLKRLYE NDNDPKKAFA GKNSLDKQPI | |
| | WLDKEQMRKV PEKVKIVTLE AIYTIRKEIS PDLKVDKVID VGVRKILIDR | |
| | LNEYGNDAKK AFSNLDKNPI WLNKEKGISI KRVTISGISN AQSLHVKKDK | |
| | DGKPILDENG RNIPVDFVNT GNNHHVAVYY RPVIDKRGQL VVDEAGNPKY | |
| | ELEEVVVSFF EAVTRANLGL PIIDKDYKTT EGWQFLFSMK QNEYFVEPNE | |
| | KTGFNPKEID LLDVENYGLI SPNLFRVQKF SLKNYVFRHH LETTIKDTSS | |
| | ILRGITWIDF RSSKGLDTIV KVRVNHIGQI VSVGEY | |
| AOL40912.1 | METQTSNQLI TSHLKDYPKQ DYFVGLDIGT NSVGWAVTNT SYELLKFHSH | (SEQ |
| Veillonella | KMWGSRLFEE GESAVTRRGF RSMRRRLERR KLRLKLLEEL FADAMAQVDS | ID |
| atypica | TFFIRLHESK YHYEDKTTGH SSKHILFIDE DYTDQDYFTE YPTIYHLRKD | NO: |
| ACS-134- | LMENGTDDIR KLFLAVHHIL KYRGNFLYEG ATFNSNAFTF EDVLKQALVN | 50) |
| V-Col7a | ITFNCFDTNS AISSISNILM ESGKTKSDKA KAIERLVDTY TVFDEVNTPD | |
| | KPQKEQVKED KKTLKAFANL VLGLSANLID LFGSVEDIDD DLKKLQIVGD | |
| | TYDEKRDELA KVWGDEIHII DDCKSVYDAI ILMSIKEPGL TISQSKVKAF | |
| | DKHKEDLVIL KSLLKLDRNV YNEMFKSDKK GLHNYVHYIK QGRTEETSCS | |
| | REDFYKYTKK IVEGLADSKD KEYILNEIEL QTLLPLQRIK DNGVIPYQLH | |
| | LEELKVILDK CGPKFPFLHT VSDGFSVTEK LIKMLEFRIP YYVGPLNTHH | |
| | NIDNGGFSWA VRKQAGRVTP WNFEEKIDRE KSAAAFIKNL TNKCTYLFGE | |
| | DVLPKSSLLY SEFMLLNELN NVRIDGKALA QGVKQHLIDS IFKQDHKKMT | |
| | KNRIELFLKD NNYITKKHKP EITGLDGEIK NDLTSYRDMV RILGNNEDVS | |
| | MAEDIITDIT IFGESKKMLR QTLRNKFGSQ LNDETIKKLS KLRYRDWGRL | |
| | SKKLLKGIDG CDKAGNGAPK TIIELMRNDS YNLMEILGDK FSFMECIEEE | |
| | NAKLAQGQVV NPHDIIDELA LSPAVKRAVW QALRIVDEVA HIKKALPSRI | |
| | FVEVARTNKS EKKKKDSRQK RLSDLYSAIK KDDVLQSGLQ DKEFGALKSG | |
| | LANYDDAALR SKKLYLYYTQ MGRCAYTGNI IDLNQLNTDN YDIDHIYPRS | |
| | LTKDDSFDNL VLCERTANAK KSDIYPIDNR IQTKQKPFWA FLKHQGLISE | |
| | RKYERLTRIA PLTADDLSGF IARQLVETNQ SVKATTILLR RLYPDIDVVE | |
| | VKAENVSDER HNNNFIKVRS LNHHHHAKDA YLNIVVGNVY HEKFTRNERL | |
| | FFKKNGANRT YNLAKMFNYD VICTNAQDGK AWDVKTSMNT VKKMMASNDV | |
| | RVTRRLLEQS GALADATIYK ASVAAKAKDG AYIGMKTKYS VFADVTKYGG | |
| | MTKIKNAYSI IVQYTGKKGE EIKEIVPLPI YLINRNATDI ELIDYVKSVI | |
| | PKAKDISIKY RKLCINQLVK VNGFYYYLGG KINDKIYIDN AIELVVPHDI | |
| | ATYIKLLDKY DLLRKENKTL KASSITTSIY NINTSTVVSL SNKVGIDVED | |
| | YFMSKLRTPL YMKMKGNKVD ELSSTGRSKF IKMTLEEQSI YLLEVLNLLT | |
| | NSKTTFDVKP LGITGSRSTI GVKIHNLDEF KIINESITGL YSNEVTIV | |
| WP_013389026.1 | MKYSIGLDIG IASVGWSVIN KDKERIEDMG VRIFQKAENP KDGSSLASSR | (SEQ |
| Ilyobacter | REKRGSRRRN RRKKHRLDRI KNILCESGLV KKNEIEKIYK NAYLKSPWEL | ID |
| polytropus | RAKSLEAKIS NKEIAQILLH IAKRRGFKSF RKTDRNADDT GKLLSGIQEN | NO: |
| DSM 2926 | KKIMEEKGYL TIGDMVAKDP KENTHVRNKA GSYLFSFSRK LLEDEVRKIQ | 51) |
| | AKQKELGNTH FTDDVLEKYI EVENSQRNED EGPSKPSPYY SEIGQIAKMI | |
| | GNCTFESSEK RTAKNTWSGE RFVFLQKLNN FRIVGLSGKR PLTEEERDIV | |
| | EKEVYLKKEV RYEKLRKILY LKEEERFGDL NYSKDEKQDK KTEKTKFISL | |
| | IGNYTIKKLN LSEKLKSEIE EDKSKLDKII EILTENKSDK TIESNLKKLE | |
| | LSREDIEILL SEEFSGTLNL SLKAIKKILP YLEKGLSYNE ACEKADYDYK | |
| | NNGIKFKRGE LLPVVDKDLI ANPVVLRAIS QTRKVVNAII RKYGTPHTIH | |
| | VEVARDLAKS YDDRQTIIKE NKKRELENEK TKKFISEEFG IKNVKGKLLL | |
| | KYRLYQEQEG RCAYSRKELS LSEVILDESM TDIDHIIPYS RSMDDSYSNK | |
| | VLVLSGENRK KSNLLPKEYF DRQGRDWDTF VLNVKAMKIH PRKKSNLLKE | |
| | KFTREDNKDW KSRALNDTRY ISRFVANYLE NALEYRDDSP KKRVFMIPGQ | |
| | LTAQLRARWR LNKVRENGDL HHALDAAVVA VTDQKAINNI SNISRYKELK | |
| | NCKDVIPSIE YHADEETGEV YFEEVKDTRF PMPWSGEDLE LQKRLESENP | |
| | REEFYNLLSD KRYLGWFNYE EGFIEKLRPV FVSRMPNRGV KGQAHQETIR | |
| | SSKKISNQIA VSKKPLNSIK LKDLEKMQGR DTDRKLYEAL KNRLEEYDDK | |
| | PEKAFAEPFY KPTNSGKRGP LVRGIKVEEK QNVGVYVNGG QASNGSMVRI | |
| | DVFRKNGKFY TVPIYVHQTL LKELPNRAIN GKPYKDWDLI DGSFEFLYSF | |
| | YPNDLIEIEF GKSKSIKNDN KLTKTEIPEV NLSEVLGYYR GMDTSTGAAT | |
| | IDTQDGKIQM RIGIKTVKNI KKYQVDVLGN VYKVKREKRQ TF | |
| WP_005864263.1 | MKKIVGLDLG TNSIGWALIN AYINKEHLYG IEACGSRIIP MDAAILGNFD | (SEQ |
| Parabacteroides | KGNSISQTAD RTSYRGIRRL RERHLLRRER LHRILDLLGF LPKHYSDSLN | ID |
| sp. 20_3 | RYGKFLNDIE CKLPWVKDET GSYKFIFQES FKEMLANFTE HHPILIANNK | NO: |
| | KVPYDWTIYY LRKKALTQKI SKEELAWILL NFNQKRGYYQ LRGEEEETPN | 52) |
| | KLVEYYSLKV EKVEDSGERK GKDTWYNVHL ENGMIYRRTS NIPLDWEGKT | |
| | KEFIVTTDLE ADGSPKKDKE GNIKRSFRAP KDDDWTLIKK KTEADIDKIK | |
| | MTVGAYIYDT LLQKPDQKIR GKLVRTIERK YYKNELYQIL KTQSEFHEEL | |
| | RDKQLYIACL NELYPNNEPR RNSISTRDFC HLFIEDIIFY QRPLKSKKSL | |
| | IDNCPYEENR YIDKESGEIK HASIKCIAKS HPLYQEFRLW QFIVNLRIYR | |
| | KETDVDVTQE LLPTEADYVT LFEWLNEKKE IDQKAFFKYP PFGFKKTTSN | |
| | YRWNYVEDKP YPCNETHAQI IARLGKAHIP KAFLSKEKEE TLWHILYSIE | |
| | DKQEIEKALH SFANKNNLSE EFIEQFKNEP PFKKEYGSYS AKAIKKLLPL | |
| | MRMGKYWSIE NIDNGTRIRI NKIIDGEYDE NIRERVRQKA INLTDITHER | |
| | ALPLWLACYL VYDRHSEVKD IVKWKTPKDI DLYLKSFKQH SLRNPIVEQV | |
| | ITETLRTVRD IWQQVGHIDE IHIELGREMK NPADKRARMS QQMIKNENTN | |
| | LRIKALLTEF LNPEFGIENV RPYSPSQQDL LRIYEEGVLN SILELPEDIG | |
| | IILGKFNQTD TLKRPTRSEI LRYKLWLEQK YRSPYTGEMI PLSKLFTPAY | |
| | EIEHIIPQSR YFDDSLSNKV ICESEINKLK DRSLGYEFIK NHHGEKVELA | |
| | FDKPVEVLSV EAYEKLVHES YSHNRSKMKK LLMEDIPDQF IERQLNDSRY | |
| | ISKVVKSLLS NIVREENEQE AISKNVIPCT GGITDRLKKD WGINDVWNKI | |
| | VLPRFIRLNE LTESTRETSI NINNTMIPSM PLELQKGENK KRIDHRHHAM | |
| | DAIIIACANR NIVNYLNNVS ASKNTKITRR DLQTLLCHKD KTDNNGNYKW | |
| | VIDKPWETFT QDTLTALQKI TVSFKQNLRV INKTTNHYQH YENGKKIVSN | |
| | QSKGDSWAIR KSMHKETVHG EVNLRMIKTV SFNEALKKPQ AIVEMDLKKK | |
| | ILAMLELGYD TKRIKNYFEE NKDTWQDINP SKIKVYYFTK ETKDRYFAVR | |
| | KPIDTSFDKK KIKESITDTG IQQIMLRHLE TKDNDPTLAF SPDGIDEMNR | |
| | NILILNKGKK HQPIYKVRVY EKAEKFTVGQ KGNKRTKFVE AAKGTNLFFA | |
| | IYETEEIDKD TKKVIRKRSY STIPLNVVIE RQKQGLSSAP EDENGNLPKY | |
| | ILSPNDLVYV PTQEEINKGE VVMPIDRDRI YKMVDSSGIT ANFIPASTAN | |
| | LIFALPKATA EIYCNGENCI QNEYGIGSPQ SKNQKAITGE MVKEICFPIK | |
| | VDRLGNIIQV GSCILTN | |
| GAP01010.1 | MVYDVGLDIG TGSVGWVALD ENGKLARAKG KNLVGVRLFD TAQTAADRRG | (SEQ |
| Fructobacillus | FRTTRRRLSR RKWRLRLLDE LFSAEINEID SSFFQRLKYS YVHPKDEENK | ID |
| fructosus | AHYYGGYLFP TEEETKKFHR SYPTIYHLRQ ELMAQPNKRF DIREIYLAIH | NO: |
| KCTC 3544 | HLVKYRGHFL SSQEKITIGS TYNPEDLANA IEVYADEKGL SWELNNPEQL | 53) |
| | TEIISGEAGY GLNKSMKADE ALKLFEFDNN QDKVAIKTLL AGLTGNQIDF | |
| | AKLFGKDISD KDEAKLWKLK LDDEALEEKS QTILSQLTDE EIELFHAVVQ | |
| | AYDGFVLIGL LNGADSVSAA MVQLYDQHRE DRKLLKSLAQ KAGLKHKRFS | |
| | EIYEQLALAT DEATIKNGIS TARELVEESN LSKEVKEDTL RRLDENEFLP | |
| | KQRTKANSVI PHQLHLAELQ KILQNQGQYY PFLLDTFEKE DGQDNKIEEL | |
| | LRFRIPYYVG PLVTKKDVEH AGGDADNHWV ERNEGFEKSR VTPWNEDKVF | |
| | NRDKAARDFI ERLTGNDTYL IGEKTLPQNS LRYQLFTVLN ELNNVRVNGK | |
| | KFDSKTKADL INDLFKARKT VSLSALKDYL KAQGKGDVTI TGLADESKEN | |
| | SSLSSYNDLK KTFDAEYLEN EDNQETLEKI IEIQTVFEDS KIASRELSKL | |
| | PLDDDQVKKL SQTHYTGWGR LSEKLLDSKI IDERGQKVSI LDKLKSTSQN | |
| | FMSIINNDKY GVQAWITEQN TGSSKLTFDE KVNELTTSPA NKRGIKQSFA | |
| | VLNDIKKAMK EEPRRVYLEF AREDQTSVRS VPRYNQLKEK YQSKSLSEEA | |
| | KVLKKTLDGN KNKMSDDRYF LYFQQQGKDM YTGRPINFER LSQDYDIDHI | |
| | IPQAFTKDDS LDNRVLVSRP ENARKSDSFA YTDEVQKQDG SLWTSLLKSG | |
| | FINRKKYERL TKAGKYLDGQ KTGFIARQLV ETRQIIKNVA SLIEGEYENS | |
| | KAVAIRSEIT ADMRLLVGIK KHREINSFHH AFDALLITAA GQYMQNRYPD | |
| | RDSTNVYNEF DRYTNDYLKN LRQLSSRDEV RRLKSFGFVV GTMRKGNEDW | |
| | SEENTSYLRK VMMFKNILTT KKTEKDRGPL NKETIFSPKS GKKLIPLNSK | |
| | RSDTALYGGY SNVYSAYMTL VRANGKNLLI KIPISIANQI EVGNLKINDY | |
| | IVNNPAIKKE EKILISKLPL GQLVNEDGNL IYLASNEYRH NAKQLWLSTT | |
| | DADKIASISE NSSDEELLEA YDILTSENVK NRFPFFKKDI DKLSQVRDEF | |
| | LDSDKRIAVI QTILRGLQID AAYQAPVKII SKKVSDWHKL QQSGGIKLSD | |
| | NSEMIYQSAT GIFETRVKIS DLL | |
| Bacillus | MNYKMGLDIG IASVGWAVIN LDLKRIEDLG VRIFDKAEHP QNGESLALPR | (SEQ |
| smithii | RIARSARRRL RRRKHRLERI RRLLVSENVL TKEEMNLLFK QKKQIDVWQL | ID |
| WP_003354196.1 | RVDALERKLN NDELARVLLH LAKRRGFKSN RKSERNSKES SEFLKNIEEN | NO: |
| | QSILAQYRSV GEMIVKDSKF AYHKRNKLDS YSNMIARDDL EREIKLIFEK | 54) |
| | QREFNNPVCT ERLEEKYLNI WSSQRPFASK EDIEKKVGFC TFEPKEKRAP | |
| | KATYTFQSFI VWEHINKLRL VSPDETRALT EIERNLLYKQ AFSKNKMTYY | |
| | DIRKLLNLSD DIHFKGLLYD PKSSLKQIEN IRFLELDSYH KIRKCIENVY | |
| | GKDGIRMFNE TDIDTFGYAL TIFKDDEDIV AYLQNEYITK NGKRVSNLAN | |
| | KVYDKSLIDE LLNLSFSKFA HLSMKAIRNI LPYMEQGEIY SKACELAGYN | |
| | FTGPKKKEKA LLLPVIPNIA NPVVMRALTQ SRKVVNAIIK KYGSPVSIHI | |
| | ELARDLSHSF DERKKIQKDQ TENRKKNETA IKQLIEYELT KNPTGLDIVK | |
| | FKLWSEQQGR CMYSLKPIEL ERLLEPGYVE VDHILPYSRS LDDSYANKVL | |
| | VLTKENREKG NHTPVEYLGL GSERWKKFEK FVLANKQFSK KKKQNLLRLR | |
| | YEETEEKEFK ERNLNDTRYI SKFFANFIKE HLKFADGDGG QKVYTINGKI | |
| | TAHLRSRWDF NKNREESDLH HAVDAVIVAC ATQGMIKKIT EFYKAREQNK | |
| | ESAKKKEPIF PQPWPHFADE LKARLSKFPQ ESIEAFALGN YDRKKLESLR | |
| | PVFVSRMPKR SVTGAAHQET LRRCVGIDEQ SGKIQTAVKT KLSDIKLDKD | |
| | GHFPMYQKES DPRTYEAIRQ RLLEHNNDPK KAFQEPLYKP KKNGEPGPVI | |
| | RTVKIIDTKN KVVHLDGSKT VAYNSNIVRT DVFEKDGKYY CVPVYTMDIM | |
| | KGTLPNKAIE ANKPYSEWKE MTEEYTFQFS LFPNDLVRIV LPREKTIKTS | |
| | TNEEIIIKDI FAYYKTIDSA TGGLELISHD RNFSLRGVGS KTLKRFEKYQ | |
| | VDVLGNIHKV KGEKRVGLAA PTNQKKGKTV DSLQSVSD | |
| Mycoplasma | MEKKRKVTLG FDLGIASVGW AIVDSETNQV YKLGSRLFDA PDTNLERRTQ | (SEQ |
| canis PG | RGTRRLLRRR KYRNQKFYNL VKRTEVFGLS SREAIENRFR ELSIKYPNII | ID |
| 14 | ELKTKALSQE VCPDEIAWIL HDYLKNRGYF YDEKETKEDF DQQTVESMPS | NO: |
| EIE39736.1 | YKLNEFYKKY GYFKGALSQP TESEMKDNKD LKEAFFFDES NKEWLKEINY | 55) |
| WP_004794730.1 | FENVQKNILS ETFIEEFKKI FSFTRDISKG PGSDNMPSPY GIFGEFGDNG | |
| | QGGRYEHIWD KNIGKCSIFT NEQRAPKYLP SALIFNFLNE LANIRLYSTD | |
| | KKNIQPLWKL SSVDKLNILL NLFNLPISEK KKKLTSTNIN DIVKKESIKS | |
| | IMISVEDIDM IKDEWAGKEP NVYGVGLSGL NIEESAKENK FKFQDLKILN | |
| | VLINLLDNVG IKFEFKDRND IIKNLELLDN LYLFLIYQKE SNNKDSSIDL | |
| | FIAKNESLNI ENLKLKLKEF LLGAGNEFEN HNSKTHSLSK KAIDEILPKL | |
| | LDNNEGWNLE AIKNYDEEIK SQIEDNSSLM AKQDKKYLND NFLKDAILPP | |
| | NVKVTFQQAI LIFNKIIQKF SKDFEIDKVV IELAREMTQD QENDALKGIA | |
| | KAQKSKKSLV EERLEANNID KSVENDKYEK LIYKIFLWIS QDFKDPYTGA | |
| | QISVNEIVNN KVEIDHIIPY SLCFDDSSAN KVLVHKQSNQ EKSNSLPYEY | |
| | IKQGHSGWNW DEFTKYVKRV FVNNVDSILS KKERLKKSEN LLTASYDGYD | |
| | KLGFLARNLN DTRYATILFR DQLNNYAEHH LIDNKKMFKV IAMNGAVTSF | |
| | IRKNMSYDNK LRLKDRSDFS HHAYDAAIIA LFSNKTKTLY NLIDPSLNGI | |
| | ISKRSEGYWV IEDRYTGEIK ELKKEDWTSI KNNVQARKIA KEIEEYLIDL | |
| | DDEVFFSRKT KRKTNRQLYN ETIYGIATKT DEDGITNYYK KEKFSILDDK | |
| | DIYLRLLRER EKFVINQSNP EVIDQIIEII ESYGKENNIP SRDEAINIKY | |
| | TKNKINYNLY LKQYMRSLTK SLDQFSEEFI NQMIANKTFV LYNPTKNTTR | |
| | KIKFLRLVND VKINDIRKNQ VINKENGKNN EPKAFYENIN SLGAIVEKNS | |
| | ANNFKTLSIN TQIAIFGDKN WDIEDFKTYN MEKIEKYKEI YGIDKTYNFH | |
| | SFIFPGTILL DKQNKEFYYI SSIQTVRDII EIKFLNKIEF KDENKNQDTS | |
| | KTPKRLMFGI KSIMNNYEQV DISPFGINKK IFE | |
| Odoribacter | METTLGIDLG TNSIGLALVD QEEHQILYSG VRIFPEGINK DTIGLGEKEE | (SEQ |
| laneus YIT | SRNATRRAKR QMRRQYFRKK LRKAKLLELL IAYDMCPLKP EDVRRWKNWD | ID |
| EHP49880.1 | KQQKSTVRQF PDTPAFREWL KQNPYELRKQ AVTEDVTRPE LGRILYQMIQ | NO: |
| | RRGFLSSRKG KEEGKIFTGK DRMVGIDETR KNLQKQTLGA YLYDIAPKNG | 56) |
| | EKYRFRTERV RARYTLRDMY IREFEIIWQR QAGHLGLAHE QATRKKNIFL | |
| | EGSATNVRNS KLITHLQAKY GRGHVLIEDT RITVTFQLPL KEVLGGKIEI | |
| | EEEQLKFKSN ESVLFWQRPL RSQKSLLSKC VFEGRNFYDP VHQKWIIAGP | |
| | TPAPLSHPEF EEFRAYQFIN NIIYGKNEHL TAIQREAVFE LMCTESKDEN | |
| | FEKIPKHLKL FEKFNEDDTT KVPACTTISQ LRKLFPHPVW EEKREEIWHC | |
| | FYFYDDNTLL FEKLQKDYAL QTNDLEKIKK IRLSESYGNV SLKAIRRINP | |
| | YLKKGYAYST AVLLGGIRNS FGKRFEYFKE YEPEIEKAVC RILKEKNAEG | |
| | EVIRKIKDYL VHNRFGFAKN DRAFQKLYHH SQAITTQAQK ERLPETGNLR | |
| | NPIVQQGLNE LRRTVNKLLA TCREKYGPSF KFDHIHVEMG RELRSSKTER | |
| | EKQSRQIREN EKKNEAAKVK LAEYGLKAYR DNIQKYLLYK EIEEKGGTVC | |
| | CPYTGKTLNI SHTLGSDNSV QIEHIIPYSI SLDDSLANKT LCDATENREK | |
| | GELTPYDFYQ KDPSPEKWGA SSWEEIEDRA FRLLPYAKAQ RFIRRKPQES | |
| | NEFISRQLND TRYISKKAVE YLSAICSDVK AFPGQLTAEL RHLWGLNNIL | |
| | QSAPDITFPL PVSATENHRE YYVITNEQNE VIRLFPKQGE TPRTEKGELL | |
| | LTGEVERKVF RCKGMQEFQT DVSDGKYWRR IKLSSSVTWS PLFAPKPISA | |
| | DGQIVLKGRI EKGVFVCNQL KQKLKTGLPD GSYWISLPVI SQTFKEGESV | |
| | NNSKLTSQQV QLFGRVREGI FRCHNYQCPA SGADGNEWCT LDTDTAQPAF | |
| | TPIKNAPPGV GGGQIILTGD VDDKGIFHAD DDLHYELPAS LPKGKYYGIF | |
| | TVESCDPTLI PIELSAPKTS KGENLIEGNI WVDEHTGEVR FDPKKNREDQ | |
| | RHHAIDAIVI ALSSQSLFQR LSTYNARREN KKRGLDSTEH FPSPWPGFAQ | |
| | DVRQSVVPLL VSYKQNPKTL CKISKTLYKD GKKIHSCGNA VRGQLHKETV | |
| | YGQRTAPGAT EKSYHIRKDI RELKTSKHIG KVVDITIRQM LLKHLQENYH | |
| | IDITQEFNIP SNAFFKEGVY RIFLPNKHGE PVPIKKIRMK EELGNAERLK | |
| | DNINQYVNPR NNHHVMIYQD ADGNLKEEIV SFWSVIERQN QGQPIYQLPR | |
| | EGRNIVSILQ INDTFLIGLK EEEPEVYRND LSTLSKHLYR VQKLSGMYYT | |
| | FRHHLASTLN NEREEFRIQS LEAWKRANPV KVQIDEIGRI TFLNGPLC | |
| Akkermansia | MSRSLTFSFD IGYASIGWAV IASASHDDAD PSVCGCGTVL FPKDDCQAFK | (SEQ |
| muciniphila | RREYRRLRRN IRSRRVRIER IGRLLVQAQI ITPEMKETSG HPAPFYLASE | ID |
| ATCC | ALKGHRTLAP IELWHVLRWY AHNRGYDNNA SWSNSLSEDG GNGEDTERVK | NO: |
| BAA-835 | HAQDLMDKHG TATMAETICR ELKLEEGKAD APMEVSTPAY KNLNTAFPRL | 57) |
| WP_012421034.1 | IVEKEVRRIL ELSAPLIPGL TAEIIELIAQ HHPLTTEQRG VLLQHGIKLA | |
| | RRYRGSLLFG QLIPREDNRI ISRCPVTWAQ VYEAELKKGN SEQSARERAE | |
| | KLSKVPTANC PEFYEYRMAR ILCNIRADGE PLSAEIRREL MNQARQEGKL | |
| | TKASLEKAIS SRLGKETETN VSNYFTLHPD SEEALYLNPA VEVLQRSGIG | |
| | QILSPSVYRI AANRLRRGKS VTPNYLLNLL KSRGESGEAL EKKIEKESKK | |
| | KEADYADTPL KPKYATGRAP YARTVLKKVV EEILDGEDPT RPARGEAHPD | |
| | GELKAHDGCL YCLLDTDSSV NQHQKERRLD TMTNNHLVRH RMLILDRLLK | |
| | DLIQDFADGQ KDRISRVCVE VGKELTTESA MDSKKIQREL TLRQKSHTDA | |
| | VNRLKRKLPG KALSANLIRK CRIAMDMNWT CPFTGATYGD HELENLELEH | |
| | IVPHSFRQSN ALSSLVLTWP GVNRMKGQRT GYDFVEQEQE NPVPDKPNLH | |
| | ICSLNNYREL VEKLDDKKGH EDDRRRKKKR KALLMVRGLS HKHQSQNHEA | |
| | MKEIGMTEGM MTQSSHLMKL ACKSIKTSLP DAHIDMIPGA VTAEVRKAWD | |
| | VFGVFKELCP EAADPDSGKI LKENLRSLTH LHHALDACVL GLIPYIIPAH | |
| | HNGLLRRVLA MRRIPEKLIP QVRPVANQRH YVINDDGRMM LRDLSASLKE | |
| | NIREQLMEQR VIQHVPADMG GALLKETMQR VLSVDGSGED AMVSLSKKKD | |
| | GKKEKNQVKA SKLVGVFPEG PSKLKALKAA IEIDGNYGVA LDPKPVVIRH | |
| | IKVFKRIMAL KEQNGGKPVR ILKKGMLIHL TSSKDPKHAG VWRIESIQDS | |
| | KGGVKLDLQR AHCAVPKNKT HECNWREVDL ISLLKKYQMK RYPTSYTGTP | |
| | R | |
| Dinoroseobacter | MRLGLDIGTS SIGWWLYETD GAGSDARITG VVDGGVRIFS DGRDPKSGAS | (SEQ |
| shibae | LAVDRRAARA MRRRRDRYLR RRATLMKVLA ETGLMPADPA EAKALEALDP | ID |
| DFL 12 = | FALRAAGLDE PLPLPHLGRA LFHLNQRRGF KSNRKTDRGD NESGKIKDAT | NO: |
| DSM 16493 | ARLDMEMMAN GARTYGEFLH KRRQKATDPR HVPSVRTRLS IANRGGPDGK | 58) |
| WP_012177079.1 | EEAGYDFYPD RRHLEEEFHK LWAAQGAHHP ELTETLRDLL FEKIFFQRPL | |
| | KEPEVGLCLF SGHHGVPPKD PRLPKAHPLT QRRVLYETVN QLRVTADGRE | |
| | ARPLTREERD QVIHALDNKK PTKSLSSMVL KLPALAKVLK LRDGERFTLE | |
| | TGVRDAIACD PLRASPAHPD RFGPRWSILD ADAQWEVISR IRRVQSDAEH | |
| | AALVDWLTEA HGLDRAHAEA TAHAPLPDGY GRLGLTATTR ILYQLTADVV | |
| | TYADAVKACG WHHSDGRTGE CFDRLPYYGE VLERHVIPGS YHPDDDDITR | |
| | FGRITNPTVH IGLNQLRRLV NRIIETHGKP HQIVVELARD LKKSEEQKRA | |
| | DIKRIRDTTE AAKKRSEKLE ELEIEDNGRN RMLLRLWEDL NPDDAMRRFC | |
| | PYTGTRISAA MIFDGSCDVD HILPYSRTLD DSFPNRTLCL REANRQKRNQ | |
| | TPWQAWGDTP HWHAIAANLK NLPENKRWRF APDAMTRFEG ENGFLDRALK | |
| | DTQYLARISR SYLDTLFTKG GHVWVVPGRF TEMLRRHWGL NSLLSDAGRG | |
| | AVKAKNRTDH RHHAIDAAVI AATDPGLLNR ISRAAGQGEA AGQSAELIAR | |
| | DTPPPWEGFR DDLRVRLDRI IVSHRADHGR IDHAARKQGR DSTAGQLHQE | |
| | TAYSIVDDIH VASRTDLLSL KPAQLLDEPG RSGQVRDPQL RKALRVATGG | |
| | KTGKDFENAL RYFASKPGPY QAIRRVRIIK PLQAQARVPV PAQDPIKAYQ | |
| | GGSNHLFEIW RLPDGEIEAQ VITSFEAHTL EGEKRPHPAA KRLLRVHKGD | |
| | MVALERDGRR VVGHVQKMDI ANGLFIVPHN EANADTRNND KSDPFKWIQI | |
| | GARPAIASGI RRVSVDEIGR LRDGGTRPI | |
| Wolinella | MIERILGVDL GISSLGWAIV EYDKDDEAAN RIIDCGVRLF TAAETPKKKE | (SEQ |
| succinogenes | SPNKARREAR GIRRVLNRRR VRMNMIKKLF LRAGLIQDVD LDGEGGMFYS | ID |
| DSM 1740 | KANRADVWEL RHDGLYRLLK GDELARVLIH IAKHRGYKFI GDDEADEESG | NO: |
| WP_011139289.1 | KVKKAGVVLR QNFEAAGCRT VGEWLWRERG ANGKKRNKHG DYEISIHRDL | 59) |
| | LVEEVEAIFV AQQEMRSTIA TDALKAAYRE IAFFVRPMQR IEKMVGHCTY | |
| | FPEERRAPKS APTAEKFIAI SKFFSTVIID NEGWEQKIIE RKTLEELLDF | |
| | AVSREKVEFR HLRKELDLSD NEIFKGLHYK GKPKTAKKRE ATLFDPNEPT | |
| | ELEFDKVEAE KKAWISLRGA AKLREALGNE FYGRFVALGK HADEATKILT | |
| | YYKDEGQKRR ELTKLPLEAE MVERLVKIGF SDFLKLSLKA IRDILPAMES | |
| | GARYDEAVLM LGVPHKEKSA ILPPLNKTDI DILNPTVIRA FAQFRKVANA | |
| | LVRKYGAFDR VHFELAREIN TKGEIEDIKE SQRKNEKERK EAADWIAETS | |
| | FQVPLTRKNI LKKRLYIQQD GRCAYTGDVI ELERLFDEGY CEIDHILPRS | |
| | RSADDSFANK VLCLARANQQ KTDRTPYEWF GHDAARWNAF ETRTSAPSNR | |
| | VRTGKGKIDR LLKKNFDENS EMAFKDRNLN DTRYMARAIK TYCEQYWVFK | |
| | NSHTKAPVQV RSGKLTSVLR YQWGLESKDR ESHTHHAVDA IIIAFSTQGM | |
| | VQKLSEYYRF KETHREKERP KLAVPLANER DAVEEATRIE NTETVKEGVE | |
| | VKRLLISRPP RARVTGQAHE QTAKPYPRIK QVKNKKKWRL APIDEEKFES | |
| | FKADRVASAN QKNFYETSTI PRVDVYHKKG KFHLVPIYLH EMVLNELPNL | |
| | SLGTNPEAMD ENFFKFSIFK DDLISIQTQG TPKKPAKIIM GYFKNMHGAN | |
| | MVLSSINNSP CEGFTCTPVS MDKKHKDKCK LCPEENRIAG RCLQGFLDYW | |
| | RSAKKLVKK EFECDQGVKF ALDVKKYQID PLGYYYEVKQ EKRLGTIPQM | |
| | SQEGLRPPRK | |
| Parasutterella | MGKTHIIGVG LDLGGTYTGT FITSHPSDEA EHRDHSSAFT VVNSEKLSES | (SEQ |
| excrementihominis | SKSRTAVRHR VRSYKGFDLR RRLLLLVAEY QLLQKKQTLA PEERENLRIA | ID |
| YIT | LSGYLKRRGY ARTEAETDTS VLESLDPSVF SSAPSFTNFF NDSEPLNIQW | NO: |
| 11859 | EAIANSPETT KALNKELSGQ KEADFKKYIK TSFPEYSAKE ILANYVEGRR | 60) |
| WP_008864843.1 | AILDASKYIA NLQSLGHKHR SKYLSDILQD MKRDSRITRL SEAFGSTDNL | |
| | WRIIGNISNL QERAVRWYFN DAKFEQGQEQ LDAVKLKNVL VRALKYLRSD | |
| | DKEWSASQKQ IIQSLEQSGD VLDVLAGLDP DRTIPPYEDQ NNRRPPEDQT | |
| | LYLNPKALSS EYGEKWKSWA NKFAGAYPLL TEDLTEILKN TDRKSRIKIR | |
| | SDVLPDSDYR LAYILQRAFD RSIALDECSI RRTAEDFENG VVIKNEKLED | |
| | VLSGHQLEEF LEFANRYYQE TAKAKNGLWF PENALLERAD LHPPMKNKIL | |
| | NVIVGQALGV SPAEGTDFIE EIWNSKVKGR STVRSICNAI ENERKTYGPY | |
| | FSEDYKFVKT ALKEGKTEKE LSKKFAAVIK VLKMVSEVVP FIGKELRLSD | |
| | EAQSKFDNLY SLAQLYNLIE TERNGFSKVS LAAHLENAWR MTMTDGSAQC | |
| | CRLPADCVRP FDGFIRKAID RNSWEVAKRI AEEVKKSVDF TNGTVKIPVA | |
| | IEANSENFTA SLTDLKYIQL KEQKLKKKLE DIQRNEENQE KRWLSKEERI | |
| | RADSHGICAY TGRPLDDVGE IDHIIPRSLT LKKSESIYNS EVNLIFVSAQ | |
| | GNQEKKNNIY LLSNLAKNYL AAVFGTSDLS QITNEIESTV LQLKAAGRLG | |
| | YFDLLSEKER ACARHALFLN SDSEARRAVI DVLGSRRKAS VNGTQAWFVR | |
| | SIFSKVRQAL AAWTQETGNE LIFDAISVPA ADSSEMRKRF AEYRPEFRKP | |
| | KVQPVASHSI DAMCIYLAAC SDPFKTKRMG SQLAIYEPIN FDNLFTGSCQ | |
| | VIQNTPRNFS DKINIANSPI FKETIYAERF LDIIVSRGEI FIGYPSNMPF | |
| | EEKPNRISIG GKDPFSILSV LGAYLDKAPS SEKEKLTIYR VVKNKAFELF | |
| | SKVAGSKFTA EEDKAAKILE ALHFVTVKQD VAATVSDLIK SKKELSKDSI | |
| | ENLAKQKGCL KKVEYSSKEF KFKGSLIIPA AVEWGKVLWN VFKENTAEEL | |
| | KDENALRKAL EAAWPSSFGT RNLHSKAKRV FSLPVVATQS GAVRIRRKTA | |
| | FGDFVYQSQD TNNLYSSFPV KNGKLDWSSP IIHPALQNRN LTAYGYRFVD | |
| | HDRSISMSEF REVYNKDDLM RIELAQGTSS RRYLRVEMPG EKFLAWFGEN | |
| | SISLGSSFKE SVSEVFDNKI YTENAEFTKF LPKPREDNKH NGTIFFELVG | |
| | PRVIFNYIVG GAASSLKEIF SEAGKERS | |
| Streptococcus | MTKFNKNYSI GLDIGVSSVG YAVVTEDYRV PAFKFKVLGN TEKEKIKKNL | (SEQ |
| sanguinis | IGSTTFVSAQ PAKGTRVFRV NRRRIDRRNH RITYLRDIFQ KEIEKVDKNF | ID |
| SK49 | YRRLDESFRV LGDKSEDLQI KQPFFGDKEL ETAYHKKYPT IYHLRKHLAD | NO: |
| WP_002933589.1 | ADKNSPVADI REVYMAISHI LKYRGHELTL DKINPNNINM QNSWIDFIES | 61) |
| | CQEVEDLEIS DESKNIADIF KSSENRQEKV KKILPYFQQE LLKKDKSIFK | |
| | QLLQLLFGLK TKFKDCFELE EEPDLNESKE NYDENLENFL GSLEEDFSDV | |
| | FAKLKVLRDT ILLSGMLTYT GATHARFSAT MVERYEEHRK DLQRFKFFIK | |
| | QNLSEQDYLD IFGRKTQNGF DVDKETKGYV GYITNKMVLT NPQKQKTIQQ | |
| | NFYDYISGKI TGIEGAEYFL NKISDGTFLR KLRTSDNGAI PNQIHAYELE | |
| | KIIERQGKDY PFLLENKDKL LSILTFKIPY YVGPLAKGSN SRFAWIKRAT | |
| | SSDILDDNDE DTRNGKIRPW NYQKLINMDE TRDAFITNLI GNDIILLNEK | |
| | VLPKRSLIYE EVMLQNELTR VKYKDKYGKA HFFDSELRQN IINGLFKNNS | |
| | KRVNAKSLIK YLSDNHKDLN AIEIVSGVEK GKSENSTLKT YNDLKTIFSE | |
| | ELLDSEIYQK ELEEIIKVIT VEDDKKSIKN YLTKFFGHLE ILDEEKINQL | |
| | SKLRYSGWGR YSAKLLLDIR DEDTGENLLQ FLRNDEENRN LTKLISDNTL | |
| | SFEPKIKDIQ SKSTIEDDIF DEIKKLAGSP AIKRGILNSI KIVDELVQII | |
| | GYPPHNIVIE MARENMTTEE GQKKAKTRKT KLESALKNIE NSLLENGKVP | |
| | HSDEQLQSEK LYLYYLQNGK DMYTLDKTGS PAPLYLDQLD QYEVDHIIPY | |
| | SFLPIDSIDN KVLTHRENNQ QKLNNIPDKE TVANMKPFWE KLYNAKLISQ | |
| | TKYQRLTTSE RTPDGVLTES MKAGFIERQL VETRQIIKHV ARILDNRFSD | |
| | TKIITLKSQL ITNFRNTFHI AKIRELNDYH HAHDAYLAVV VGQTLLKVYP | |
| | KLAPELIYGH HAHFNRHEEN KATLRKHLYS NIMRFFNNPD SKVSKDIWDC | |
| | NRDLPIIKDV IYNSQINFVK RTMIKKGAFY NQNPVGKENK QLAANNRYPL | |
| | KTKALCLDTS IYGGYGPMNS ALSIIIIAER FNEKKGKIET VKEFHDIFII | |
| | DYEKENNNPF QFLNDTSENG FLKKNNINRV LGFYRIPKYS LMQKIDGTRM | |
| | LFESKSNLHK ATQFKLIKTQ NELFFHMKRL LTKSNLMDLK SKSAIKESQN | |
| | FILKHKEEFD NISNQLSAFS QKMLGNTTSL KNLIKGYNER KIKEIDIRDE | |
| | TIKYFYDNFI KMFSFVKSGA PKDINDFFDN KCTVARMRPK PDKKLLNATL | |
| | IHQSITGLYE TRIDLSKLGE D | |
| Actinomyces | MLHCIAVIRV PPSEEPGFFE THADSCALCH HGCMTYAAND KAIRYRVGID | (SEQ |
| sp. oral | VGLRSIGFCA VEVDDEDHPI RILNSVVHVH DAGTGGPGET ESLRKRSGVA | ID |
| taxon 180 | ARARRRGRAE KQRLKKLDVL LEELGWGVSS NELLDSHAPW HIRKRLVSEY | NO: |
| str. F0310 | IEDETERRQC LSVAMAHIAR HRGWRNSFSK VDTLLLEQAP SDRMQGLKER | 62) |
| AOL41039.1 | VEDRTGLQFS EEVTQGELVA TLLEHDGDVT IRGFVRKGGK ATKVHGVLEG | |
| | KYMQSDLVAE LRQICRTQRV SETTFEKLVL SIFHSKEPAP SAARQRERVG | |
| | LDELQLALDP AAKQPRAERA HPAFQKFKVV ATLANMRIRE QSAGERSLTS | |
| | EELNRVARYL LNHTESESPT WDDVARKLEV PRHRLRGSSR ASLETGGGLT | |
| | YPPVDDTTVR VMSAEVDWLA DWWDCANDES RGHMIDAISN GCGSEPDDVE | |
| | DEEVNELISS ATAEDMLKLE LLAKKLPSGR VAYSLKTLRE VTAAILETGD | |
| | DLSQAITRLY GVDPGWVPTP APIEAPVGNP SVDRVLKQVA RWLKFASKRW | |
| | GVPQTVNIEH TREGLKSASL LEEERERWER FEARREIRQK EMYKRLGISG | |
| | PFRRSDQVRY EILDLQDCAC LYCGNEINFQ TFEVDHIIPR VDASSDSRRT | |
| | NLAAVCHSCN SAKGGLAFGQ WVKRGDCPSG VSLENAIKRV RSWSKDRLGL | |
| | TEKAMGKRKS EVISRLKTEM PYEEFDGRSM ESVAWMAIEL KKRIEGYENS | |
| | DRPEGCAAVQ VNAYSGRLTA CARRAAHVDK RVRLIRLKGD DGHHKNRFDR | |
| | RNHAMDALVI ALMTPAIART IAVREDRREA QQLTRAFESW KNFLGSEERM | |
| | QDRWESWIGD VEYACDRLNE LIDADKIPVT ENLRLRNSGK LHADQPESLK | |
| | KARRGSKRPR PQRYVLGDAL PADVINRVTD PGLWTALVRA PGFDSQLGLP | |
| | ADLNRGLKLR GKRISADFPI DYFPTDSPAL AVQGGYVGLE FHHARLYRII | |
| | GPKEKVKYAL LRVCAIDLCG IDCDDLFEVE LKPSSISMRT ADAKLKEAMG | |
| | NGSAKQIGWL VLGDEIQIDP TKFPKQSIGK FLKECGPVSS WRVSALDTPS | |
| | KITLKPRLLS NEPLLKTSRV GGHESDLVVA ECVEKIMKKT GWVVEINALC | |
| | QSGLIRVIRR NALGEVRTSP KSGLPISLNL R | |
| Rhodovulum | MGIRFAFDLG TNSIGWAVWR TGPGVFGEDT AASLDGSGVL IFKDGRNPKD | (SEQ |
| sp. PH10 | GQSLATMRRV PRQSRKRRDR FVLRRRDLLA ALRKAGLFPV DVEEGRRLAA | ID |
| WP_008386983.1 | TDPYHLRAKA LDESLTPHEM GRVIFHLNQR RGERSNRKAD RQDREKGKIA | NO: |
| | EGSKRLAETL AATNCRTLGE FLWSRHRGTP RTRSPTRIRM EGEGAKALYA | 63) |
| | FYPTREMVRA EFERLWTAQS RFAPDLLTPE RHEEIAGILF RQRDLAPPKI | |
| | GCCTFEPSER RLPRALPSVE ARGIYERLAH LRITTGPVSD RGLTRPERDV | |
| | LASALLAGKS LTFKAVRKTL KILPHALVNF EEAGEKGLDG ALTAKLLSKP | |
| | DHYGAAWHGL SFAEKDTFVG KLLDEADEER LIRRLVTENR LSEDAARRCA | |
| | SIPLADGYGR LGRTANTEIL AALVEETDET GTVVTYAEAV RRAGERTGRN | |
| | WHHSDERDGV ILDRLPYYGE ILQRHVVPGS GEPEEKNEAA RWGRLANPTV | |
| | HIGLNQLRKV VNRLIAAHGR PDQIVVELAR ELKLNREQKE RLDRENRKNR | |
| | EENERRTAIL AEHGQRDTAE NKIRLRLFEE QARANAGIAL CPYTGRAIGI | |
| | AELFTSEVEI DHILPVSLTL DDSLANRVLC RREANREKRR QTPFQAFGAT | |
| | PAWNDIVARA AKLPPNKRWR FDPAALERFE REGGELGRQL NETKYLSRLA | |
| | KIYLGKICDP DRVYVTPGTL TGLLRARWGL NSILSDSNFK NRSDHRHHAV | |
| | DAVVIGVLTR GMIQRIAHDA ARAEDQDLDR VERDVPVPFE DERDHVRERV | |
| | STITVAVKPE HGKGGALHED TSYGLVPDTD PNAALGNLVV RKPIRSLTAG | |
| | EVDRVRDRAL RARLGALAAP FRDESGRVRD AKGLAQALEA FGAENGIRRV | |
| | RILKPDASVV TIADRRTGVP YRAVAPGENH HVDIVQMRDG SWRGFAASVE | |
| | EVNRPGWRPE WEVKKLGGKL VMRLHKGDMV ELSDKDGQRR VKVVQQIEIS | |
| | ANRVRLSPHN DGGKLQDRHA DADDPFRWDL ATIPLLKDRG CVAVRVDPIG | |
| | VVTLRRSNV | |
| Bifidobacterium | MSRKNYVDDY AISLDIGNAS VGWSAFTPNY RLVRAKGHEL IGVRLFDPAD | (SEQ |
| bifidum | TAESRRMART TRRRYSRRRW RLRLLDALED QALSEIDPSF LARRKYSWVH | ID |
| S17 | PDDENNADCW YGSVLEDSNE QDKRFYEKYP TIYHLRKALM EDDSQHDIRE | NO: |
| WP_013362995.1 | IYLAIHHMVK YRGNFLVEGT LESSNAFKED ELLKLLGRIT RYEMSEGEQN | 64) |
| | SDIEQDDENK LVAPANGQLA DALCATRGSR SMRVDNALEA LSAVNDLSRE | |
| | QRAIVKAIFA GLEGNKLDLA KIFVSKEFSS ENKKILGIYF NKSDYEEKCV | |
| | QIVDSGLLDD EEREFLDRMQ GQYNAIALKQ LLGRSTSVSD SKCASYDAHR | |
| | ANWNLIKLQL RTKENEKDIN ENYGILVGWK IDSGQRKSVR GESAYENMRK | |
| | KANVFFKKMI ETSDLSETDK NRLIHDIEED KLFPIQRDSD NGVIPHQLHQ | |
| | NELKQIIKKQ GKYYPFLLDA FEKDGKQINK IEGLLTFRVP YFVGPLVVPE | |
| | DLQKSDNSEN HWMVRKKKGE ITPWNFDEMV DKDASGRKFI ERLVGTDSYL | |
| | LGEPTLPKNS LLYQEYEVLN ELNNVRLSVR TGNHWNDKRR MRLGREEKTL | |
| | LCQRLFMKGQ TVTKRTAENL LRKEYGRTYE LSGLSDESKF TSSLSTYGKM | |
| | CRIFGEKYVN EHRDLMEKIV ELQTVFEDKE TLLHQLRQLE GISEADCALL | |
| | VNTHYTGWGR LSRKLLTTKA GECKISDDFA PRKHSIIEIM RAEDRNLMEI | |
| | ITDKQLGFSD WIEQENLGAE NGSSLMEVVD DLRVSPKVKR GIIQSIRLID | |
| | DISKAVGKRP SRIFLELADD IQPSGRTISR KSRLQDLYRN ANLGKEFKGI | |
| | ADELNACSDK DLQDDRLFLY YTQLGKDMYT GEELDLDRLS SAYDIDHIIP | |
| | QAVTQNDSID NRVLVARAEN ARKTDSFTYM PQIADRMRNF WQILLDNGLI | |
| | SRVKFERLTR QNEFSEREKE RFVQRSLVET RQIMKNVATL MRQRYGNSAA | |
| | VIGLNAELTK EMHRYLGFSH KNRDINDYHH AQDALCVGIA GQFAANRGFF | |
| | ADGEVSDGAQ NSYNQYLRDY LRGYREKLSA EDRKQGRAFG FIVGSMRSQD | |
| | EQKRVNPRTG EVVWSEEDKD YLRKVMNYRK MLVTQKVGDD FGALYDETRY | |
| | AATDPKGIKG IPFDGAKQDT SLYGGFSSAK PAYAVLIESK GKTRLVNVTM | |
| | QEYSLLGDRP SDDELRKVLA KKKSEYAKAN ILLRHVPKMQ LIRYGGGLMV | |
| | IKSAGELNNA QQLWLPYEEY CYFDDLSQGK GSLEKDDLKK LLDSILGSVQ | |
| | CLYPWHRFTE EELADLHVAF DKLPEDEKKN VITGIVSALH ADAKTANLSI | |
| | VGMTGSWRRM NNKSGYTFSD EDEFIFQSPS GLFEKRVTVG ELKRKAKKEV | |
| | NSKYRTNEKR LPTLSGASQP | |
| Barnesiella | MKNILGLDLG LSSIGWSVIR ENSEEQELVA MGSRVVSLTA AELSSFTQGN | (SEQ |
| intestinihominis | GVSINSQRTQ KRTQRKGYDR YQLRRTLLRN KLDTLGMLPD DSLSYLPKLQ | ID |
| YIT | LWGLRAKAVT QRIELNELGR VLLHLNQKRG YKSIKSDFSG DKKITDYVKT | NO: |
| 11860 | VKTRYDELKE MRLTIGELFF RRLTENAFFR CKEQVYPRQA YVEEFDCIMN | 65) |
| WP_008863245.1 | CQRKFYPDIL TDETIRCIRD EIIYYQRPLK SCKYLVSRCE FEKRFYLNAA | |
| | GKKTEAGPKV SPRTSPLFQV CRLWESINNI VVKDRRNEIV FISAEQRAAL | |
| | FDFLNTHEKL KGSDLLKLLG LSKTYGYRLG EQFKTGIQGN KTRVEIERAL | |
| | GNYPDKKRLL QFNLQEESSS MVNTETGEII PMISLSFEQE PLYRLWHVLY | |
| | SIDDREQLQS VLRQKFGIDD DEVLERLSAI DLVKAGFGNK SSKAIRRILP | |
| | FLQLGMNYAE ACEAAGYNHS NNYTKAENEA RALLDRLPAI KKNELRQPVV | |
| | EKILNQMVNV VNALMEKYGR FDEIRVELAR ELKQSKEERS NTYKSINKNQ | |
| | RENEQIAKRI VEYGVPTRSR IQKYKMWEES KHCCIYCGQP VDVGDELRGF | |
| | DVEVEHIIPK SLYFDDSFAN KVCSCRSCNK EKNNRTAYDY MKSKGEKALS | |
| | DYVERVNTMY TNNQISKTKW QNLLTPVDKI SIDFIDRQLR ESQYIARKAK | |
| | EILTSICYNV TATSGSVTSF LRHVWGWDTV LHDLNEDRYK KVGLTEVIEV | |
| | NHRGSVIRRE QIKDWSKRED HRHHAIDALT IACTKQAYIQ RLNNLRAEEG | |
| | PDFNKMSLER YIQSQPHFSV AQVREAVDRI LVSFRAGKRA VTPGKRYIRK | |
| | NRKRISVQSV LIPRGALSEE SVYGVIHVWE KDEQGHVIQK QRAVMKYPIT | |
| | SINREMLDKE KVVDKRIHRI LSGRLAQYND NPKEAFAKPV YIDKECRIPI | |
| | RTVRCFAKPA INTLVPLKKD DKGNPVAWVN PGNNHHVAIY RDEDGKYKER | |
| | TVTFWEAVDR CRVGIPAIVT QPDTIWDNIL QRNDISENVL ESLPDVKWQF | |
| | VLSLQQNEMF ILGMNEEDYR YAMDQQDYAL LNKYLYRVQK LSKSDYSFRY | |
| | HTETSVEDKY DGKPNLKLSM QMGKLKRVSI KSLLGLNPHK VHISVLGEIK | |
| | EIS | |
| Aminomonas | MIGEHVRGGC LFDDHWTPNW GAFRLPNTVR TFTKAENPKD GSSLAEPRRQ | (SEQ |
| paucivorans | ARGLRRRLRR KTQRLEDLRR LLAKEGVLSL SDLETLFRET PAKDPYQLRA | ID |
| DSM 12260 | EGLDRPLSFP EWVRVLYHIT KHRGFQSNRR NPVEDGQERS RQEEEGKLLS | NO |
| WP_006299850.1 | GVGENERLLR EGGYRTAGEM LARDPKFQDH RRNRAGDYSH TLSRSLLLEE | 66) |
| | ARRLFQSQRT LGNPHASSNL EEAFLHLVAF QNPFASGEDI RNKAGHCSLE | |
| | PDQIRAPRRS ASAETFMLLQ KTGNLRLIHR RTGEERPLTD KEREQIHLLA | |
| | WKQEKVTHKT LRRHLEIPEE WLFTGLPYHR SGDKAEEKLF VHLAGIHEIR | |
| | KALDKGPDPA VWDTLRSRRD LLDSIADTLT FYKNEDEILP RLESLGLSPE | |
| | NARALAPLSF SGTAHLSLSA LGKLLPHLEE GKSYTQARAD AGYAAPPPDR | |
| | HPKLPPLEEA DWRNPVVFRA LTQTRKVVNA LVRRYGPPWC IHLETARELS | |
| | QPAKVRRRIE TEQQANEKKK QQAEREFLDI VGTAPGPGDL LKMRLWREQG | |
| | GFCPYCEEYL NPTRLAEPGY AEMDHILPYS RSLDNGWHNR VLVHGKDNRD | |
| | KGNRTPFEAF GGDTARWDRL VAWVQASHLS APKKRNLLRE DEGEEAEREL | |
| | KDRNLTDTRF ITKTAATLLR DRLTFHPEAP KDPVMTLNGR LTAFLRKQWG | |
| | LHKNRKNGDL HHALDAAVLA VASRSFVYRL SSHNAAWGEL PRGREAENGE | |
| | SLPYPAFRSE VLARLCPTRE EILLRLDQGG VGYDEAFRNG LRPVFVSRAP | |
| | SRRLRGKAHM ETLRSPKWKD HPEGPRTASR IPLKDLNLEK LERMVGKDRD | |
| | RKLYEALRER LAAFGGNGKK AFVAPFRKPC RSGEGPLVRS LRIFDSGYSG | |
| | VELRDGGEVY AVADHESMVR VDVYAKKNRF YLVPVYVADV ARGIVKNRAI | |
| | VAHKSEEEWD LVDGSFDFRF SLFPGDLVEI EKKDGAYLGY YKSCHRGDGR | |
| | LLLDRHDRMP RESDCGTFYV STRKDVLSMS KYQVDPLGEI RLVGSEKPPF | |
| | VL | |
| Ralstonia | MAEKQHRWGL DIGINSIGWA VIALIEGRPA GLVATGSRIF SDGRNPKDGS | (SEQ |
| syzygii R24 | SLAVERRGPR QMRRRRDRYL RRRDREMQAL INVGLMPGDA AARKALVTEN | ID |
| CCA84553.1 | PYVLRQRGLD QALTLPEFGR ALFHLNQRRG FQSNRKTDRA TAKESGKVKN | NO: |
| | AIAAFRAGMG NARTVGEALA RRLEDGRPVR ARMVGQGKDE HYELYIAREW | 67) |
| | IAQEFDALWA SQQRFHAEVL ADAARDRLRA ILLFQRKLLP VPVGKCFLEP | |
| | NQPRVAAALP SAQRFRLMQE LNHLRVMTLA DKRERPLSFQ ERNDLLAQLV | |
| | ARPKCGFDML RKIVFGANKE AYRFTIESER RKELKGCDTA AKLAKVNALG | |
| | TRWQALSLDE QDRLVCLLLD GENDAVLADA LREHYGLTDA QIDTLLGLSF | |
| | EDGHMRLGRS ALLRVLDALE SGRDEQGLPL SYDKAVVAAG YPAHTADLEN | |
| | GERDALPYYG ELLWRYTQDA PTAKNDAERK FGKIANPTVH IGLNQLRKLV | |
| | NALIQRYGKP AQIVVELARN LKAGLEEKER IKKQQTANLE RNERIRQKLQ | |
| | DAGVPDNREN RLRMRLFEEL GQGNGLGTPC IYSGRQISLQ RLFSNDVQVD | |
| | HILPFSKTLD DSFANKVLAQ HDANRYKGNR GPFEAFGANR DGYAWDDIRA | |
| | RAAVLPRNKR NRFAETAMQD WLHNETDFLA RQLTDTAYLS RVARQYLTAI | |
| | CSKDDVYVSP GRLTAMLRAK WGLNRVLDGV MEEQGRPAVK NRDDHRHHAI | |
| | DAVVIGATDR AMLQQVATLA ARAREQDAER LIGDMPTPWP NFLEDVRAAV | |
| | ARCVVSHKPD HGPEGGLHND TAYGIVAGPF EDGRYRVRHR VSLEDLKPGD | |
| | LSNVRCDAPL QAELEPIFEQ DDARAREVAL TALAERYRQR KVWLEELMSV | |
| | LPIRPRGEDG KTLPDSAPYK AYKGDSNYCY ELFINERGRW DGELISTFRA | |
| | NQAAYRRFRN DPARFRRYTA GGRPLLMRLC INDYIAVGTA AERTIFRVVK | |
| | MSENKITLAE HFEGGTLKQR DADKDDPFKY LTKSPGALRD LGARRIFVDL | |
| | IGRVLDPGIK GD | |
| Catenibacterium | IVDYCIGLDL GTGSVGWAVV DMNHRLMKRN GKHLWGSRLF SNAETAANRR | (SEQ |
| mitsuokai | ASRSIRRRYN KRRERIRLLR AILQDMVLEK DPTFFIRLEH TSFLDEEDKA | ID |
| DSM 15897 | KYLGTDYKDN YNLFIDEDEN DYTYYHKYPT IYHLRKALCE STEKADPRLI | NO: |
| WP_006506696.1 | YLALHHIVKY RGNFLYEGQK FNMDASNIED KLSDIFTQFT SENNIPYEDD | 68) |
| | EKKNLEILEI LKKPLSKKAK VDEVMTLIAP EKDYKSAFKE LVTGIAGNKM | |
| | NVTKMILCEP IKQGDSEIKL KFSDSNYDDQ FSEVEKDLGE YVEFVDALHN | |
| | VYSWVELQTI MGATHTDNAS ISEAMVSRYN KHHDDLKLLK DCIKNNVPNK | |
| | YFDMFRNDSE KSKGYYNYIN RPSKAPVDEF YKYVKKCIEK VDTPEAKQIL | |
| | NDIELENFLL KQNSRINGSV PYQMQLDEMI KIIDNQAEYY PILKEKREQL | |
| | LSILTFRIPY YFGPLNETSE HAWIKRLEGK ENQRILPWNY QDIVDVDATA | |
| | EGFIKRMRSY CTYFPDEEVL PKNSLIVSKY EVYNELNKIR VDDKLLEVDV | |
| | KNDIYNELFM KNKTVTEKKL KNWLVNNQCC SKDAEIKGFQ KENQFSTSLT | |
| | PWIDFTNIFG KIDQSNFDLI ENIIYDLTVF EDKKIMKRRL KKKYALPDDK | |
| | VKQILKLKYK DWSRLSKKLL DGIVADNRFG SSVTVLDVLE MSRLNLMEII | |
| | NDKDLGYAQM IEEATSCPED GKFTYEEVER LAGSPALKRG IWQSLQIVEE | |
| | ITKVMKCRPK YIYIEFERSE EAKERTESKI KKLENVYKDL DEQTKKEYKS | |
| | VLEELKGFDN TKKISSDSLF LYFTQLGKCM YSGKKLDIDS LDKYQIDHIV | |
| | PQSLVKDDSF DNRVLVVPSE NQRKLDDLVV PEDIRDKMYR FWKLLFDHEL | |
| | ISPKKFYSLI KTEYTERDEE RFINRQLVET RQITKNVTQI IEDHYSTTKV | |
| | AAIRANLSHE FRVKNHIYKN RDINDYHHAH DAYIVALIGG FMRDRYPNMH | |
| | DSKAVYSEYM KMFRKNKNDQ KRWKDGFVIN SMNYPYEVDG KLIWNPDLIN | |
| | EIKKCFYYKD CYCTTKLDQK SGQLFNLTVL SNDAHADKGV TKAVVPVNKN | |
| | RSDVHKYGGF SGLQYTIVAI EGQKKKGKKT ELVKKISGVP LHLKAASINE | |
| | KINYIEEKEG LSDVRIIKDN IPVNQMIEMD GGEYLLTSPT EYVNARQLVL | |
| | NEKQCALIAD IYNAIYKQDY DNLDDILMIQ LYIELTNKMK VLYPAYRGIA | |
| | EKFESMNENY VVISKEEKAN IIKQMLIVMH RGPQNGNIVY DDFKISDRIG | |
| | RLKTKNHNLN NIVFISQSPT GIYTKKYKL | |
| Mycoplasma | MLRLYCANNL VLNNVQNLWK YLLLLIFDKK IIFLFKIKVI LIRRYMENNN | (SEQ |
| synoviae | KEKIVIGFDL GVASVGWSIV NAETKEVIDL GVRLFSEPEK ADYRRAKRTT | ID |
| 53 | RRLLRRKKFK REKFHKLILK NAEIFGLQSR NEILNVYKDQ SSKYRNILKL | NO: |
| AOL40776.1 | KINALKEEIK PSELVWILRD YLQNRGYFYK NEKLTDEFVS NSFPSKKLHE | 69) |
| | HYEKYGFFRG SVKLDNKLDN KKDKAKEKDE EEESDAKKES EELIFSNKQW | |
| | INEIVKVFEN QSYLTESFKE EYLKLFNYVR PFNKGPGSKN SRTAYGVFST | |
| | DIDPETNKFK DYSNIWDKTI GKCSLFEEEI RAPKNLPSAL IFNLQNEICT | |
| | IKNEFTEFKN WWLNAEQKSE ILKFVFTELF NWKDKKYSDK KFNKNLQDKI | |
| | KKYLLNFALE NFNLNEEILK NRDLENDTVL GLKGVKYYEK SNATADAALE | |
| | FSSLKPLYVF IKFLKEKKLD LNYLLGLENT EILYFLDSIY LAISYSSDLK | |
| | ERNEWFKKLL KELYPKIKNN NLEIIENVED IFEITDQEKF ESFSKTHSLS | |
| | REAFNHIIPL LLSNNEGKNY ESLKHSNEEL KKRTEKAELK AQQNQKYLKD | |
| | NFLKEALVPL SVKTSVLQAI KIFNQIIKNF GKKYEISQVV IEMARELTKP | |
| | NLEKLLNNAT NSNIKILKEK LDQTEKFDDF TKKKFIDKIE NSVVFRNKLF | |
| | LWFEQDRKDP YTQLDIKINE IEDETEIDHV IPYSKSADDS WFNKLLVKKS | |
| | TNQLKKNKTV WEYYQNESDP EAKWNKFVAW AKRIYLVQKS DKESKDNSEK | |
| | NSIFKNKKPN LKFKNITKKL FDPYKDLGFL ARNLNDTRYA TKVERDQLNN | |
| | YSKHHSKDDE NKLFKVVCMN GSITSFLRKS MWRKNEEQVY RENFWKKDRD | |
| | QFFHHAVDAS IIAIFSLLTK TLYNKLRVYE SYDVQRREDG VYLINKETGE | |
| | VKKADKDYWK DQHNFLKIRE NAIEIKNVLN NVDFQNQVRY SRKANTKLNT | |
| | QLFNETLYGV KEFENNFYKL EKVNLFSRKD LRKFILEDLN EESEKNKKNE | |
| | NGSRKRILTE KYIVDEILQI LENEEFKDSK SDINALNKYM DSLPSKESEF | |
| | FSQDFINKCK KENSLILTED AIKHNDPKKV IKIKNLKFFR EDATLKNKQA | |
| | VHKDSKNQIK SFYESYKCVG FIWLKNKNDL EESIFVPINS RVIHFGDKDK | |
| | DIFDEDSYNK EKLLNEINLK RPENKKENSI NEIEFVKFVK PGALLLNFEN | |
| | QQIYYISTLE SSSLRAKIKLLNKMDKGKAVS MKKITNPDEY KIIEHVNPL | |
| | GINLNWTKKL ENNN | |
| Flavobacterium | MAKILGLDLG TNSIGWAVVE RENIDFSLID KGVRIFSEGV KSEKGIESSR | (SEQ |
| branchiophilum | AAERTGYRSA RKIKYRRKLR KYETLKVLSL NRMCPLSIEE VEEWKKSGFK | ID |
| FL-15 | DYPLNPEFLK WLSTDEESNV NPYFFRDRAS KHKVSLFELG RAFYHIAQRR | NO: |
| WP_014084151.1 | GFLSNRLDQS AEGILEEHCP KIEAIVEDLI SIDEISTNIT DYFFETGILD | 70) |
| | SNEKNGYAKD LDEGDKKLVS LYKSLLAILK KNESDFENCK SEIIERLNKK | |
| | DVLGKVKGKI KDISQAMLDG NYKTLGQYFY SLYSKEKIRN QYTSREEHYL | |
| | SEFITICKVQ GIDQINEEEK INEKKEDGLA KDLYKAIFFQ RPLKSQKGLI | |
| | GKCSFEKSKS RCAISHPDFE EYRMWTYLNT IKIGTQSDKK LRFLTQDEKL | |
| | KLVPKFYRKN DENFDVLAKE LIEKGSSFGF YKSSKKNDFF YWFNYKPTDT | |
| | VAACQVAASL KNAIGEDWKT KSFKYQTINS NKEQVSRTVD YKDLWHLLTV | |
| | ATSDVYLYEF AIDKLGLDEK NAKAFSKTKL KKDFASLSLS AINKILPYLK | |
| | EGLLYSHAVE VANIENIVDE NIWKDEKQRD YIKTQISEII ENYTLEKSRF | |
| | EIINGLLKEY KSENEDGKRV YYSKEAEQSF ENDLKKKLVL FYKSNEIENK | |
| | EQQETIFNEL LPIFIQQLKD YEFIKIQRLD QKVLIFLKGK NETGQIFCTE | |
| | EKGTAEEKEK KIKNRLKKLY HPSDIEKFKK KIIKDEFGNE KIVLGSPLTP | |
| | SIKNPMAMRA LHQLRKVLNA LILEGQIDEK TIIHIEMARE LNDANKRKGI | |
| | QDYQNDNKKF REDAIKEIKK LYFEDCKKEV EPTEDDILRY QLWMEQNRSE | |
| | IYEEGKNISI CDIIGSNPAY DIEHTIPRSR SQDNSQMNKT LCSQRENREV | |
| | KKQSMPIELN NHLEILPRIA HWKEEADNLT REIEIISRSI KAAATKEIKD | |
| | KKIRRRHYLT LKRDYLQGKY DRFIWEEPKV GFKNSQIPDT GIITKYAQAY | |
| | LKSYFKKVES VKGGMVAEFR KIWGIQESFI DENGMKHYKV KDRSKHTHHT | |
| | IDAITIACMT KEKYDVLAHA WTLEDQQNKK EARSIIEASK PWKTFKEDLL | |
| | KIEEEILVSH YTPDNVKKQA KKIVRVRGKK QFVAEVERDV NGKAVPKKAA | |
| | SGKTIYKLDG EGKKLPRLQQ GDTIRGSLHQ DSIYGAIKNP LNTDEIKYVI | |
| | RKDLESIKGS DVESIVDEVV KEKIKEAIAN KVLLLSSNAQ QKNKLVGTVW | |
| | MNEEKRIAIN KVRIYANSVK NPLHIKEHSL LSKSKHVHKQ KVYGQNDENY | |
| | AMAIYELDGK RDFELINIFN LAKLIKQGQG FYPLHKKKEI KGKIVFVPIE | |
| | KRNKRDVVLK RGQQVVFYDK EVENPKDISE IVDFKGRIYI IEGLSIQRIV | |
| | RPSGKVDEYG VIMLRYFKEA RKADDIKQDN FKPDGVFKLG ENKPTRKMNH | |
| | NQFTAFVEGI DFKVLPSGKF EKI | |
| Eubacterium | MENKQYYIGL DVGTNSVGWA VIDTSYNLLR AKGKDMWGAR LFEKANTAAE | (SEQ |
| yurii | RRTKRTSRRR SEREKARKAM LKELFADEIN RVDPSFFIRL EESKFFLDDR | ID |
| subsp. | SENNRQRYTL FNDATFTDKD YYEKYKTIFH LRSALINSDE KFDVRLVFLA | NO: |
| margaretiae | ILNLFSHRGH FLNASLKGDG DIQGMDVFYN DLVESCEYFE IELPRITNID | 71) |
| ATCC | NFEKILSQKG KSRTKILEEL SEELSISKKD KSKYNLIKLI SGLEASVVEL | |
| 43715 | YNIEDIQDEN KKIKIGFRES DYEESSLKVK EIIGDEYFDL VERAKSVHDM | |
| EFM38267.1 | GLLSNIIGNS KYLCEARVEA YENHHKDLLK IKELLKKYDK KAYNDMFRKM | |
| | TDKNYSAYVG SVNSNIAKER RSVDKRKIED LYKYIEDTAL KNIPDDNKDK | |
| | IEILEKIKLG EFLKKQLTAS NGVIPNQLQS RELRAILKKA ENYLPFLKEK | |
| | GEKNLTVSEM IIQLFEFQIP YYVGPLDKNP KKDNKANSWA KIKQGGRILP | |
| | WNFEDKVDVK GSRKEFIEKM VRKCTYISDE HTLPKQSLLY EKFMVLNEIN | |
| | NIKIDGEKIS VEAKQKIYND LFVKGKKVSQ KDIKKELISL NIMDKDSVLS | |
| | GTDTVCNAYL SSIGKFTGVF KEEINKQSIV DMIEDIIFLK TVYGDEKRFV | |
| | KEEIVEKYGD EIDKDKIKRI LGFKFSNWGN LSKSFLELEG ADVGTGEVRS | |
| | IIQSLWETNF NLMELLSSRF TYMDELEKRV KKLEKPLSEW TIEDLDDMYL | |
| | SSPVKRMIWQ SMKIVDEIQT VIGYAPKRIF VEMTRSEGEK VRTKSRKDRL | |
| | KELYNGIKED SKQWVKELDS KDESYFRSKK MYLYYLQKGR CMYSGEVIEL | |
| | DKLMDDNLYD IDHIYPRSFV KDDSLDNLVL VKKEINNRKQ NDPITPQIQA | |
| | SCQGFWKILH DQGEMSNEKY SRLTRKTQEF SDEEKLSFIN RQIVETGQAT | |
| | KCMAQILQKS MGEDVDVVES KARLVSEFRH KFELFKSRLI NDFHHANDAY | |
| | LNIVVGNSYF VKFTRNPANF IKDARKNPDN PVYKYHMDRF FERDVKSKSE | |
| | VAWIGQSEGN SGTIVIVKKT MAKNSPLITK KVEEGHGSIT KETIVGVKEI | |
| | KFGRNKVEKA DKTPKKPNLQ AYRPIKTSDE RLCNILRYGG RTSISISGYC | |
| | LVEYVKKRKT IRSLEAIPVY LGRKDSLSEE KLLNYFRYNL NDGGKDSVSD | |
| | IRLCLPFIST NSLVKIDGYL YYLGGKNDDR IQLYNAYQLK MKKEEVEYIR | |
| | KIEKAVSMSK FDEIDREKNP VLTEEKNIEL YNKIQDKFEN TVFSKRMSLV | |
| | KYNKKDLSFG DFLKNKKSKF EEIDLEKQCK VLYNIIFNLS NLKEVDLSDI | |
| | GGSKSTGKCR CKKNITNYKE FKLIQQSITG LYSCEKDLMT I | |
| Acidovorax | MAQHVFGLDI GIASVGWAIL GEQRIIDLGV RCFDKAETAK EGDPLNLTRR | (SEQ |
| ebreus | QARLLRRRLY RRAWRLTQLS RLLKRKGLIA DAKLFAKAPS YGDSAWELRR | ID |
| WP_012655176.1 | QGLDRLLTPL EWARVIYHQC KHRGFHWTSK AEEAKADSDA EGGRVKQGLA | NO: |
| | HTKALMQAKN YRSAAEMVLA EFPDAQRNKR GQYDKALSRV LLGEELALLF | 72) |
| | ATQRRLGNPH ASDFFEKLIL GDGDRKSGLF WQQKPALSGA DLLKMLGKCT | |
| | FEKGEYRAPK ASFSVERHVW LTRLNNLRIV VDGRSRPLNE AERQAALLLP | |
| | YQTETSKYKT LKNAFIKAGL WGDGVREGGL AYPSQAQIDA EKTKDPEDQF | |
| | LVKLPAWHEL RKAFKAAGHE ALWQQISTPA LDGDPTLLDQ IATVLSVYKD | |
| | GAEVVQQLRQ LALPEPAASI AVLEKISFDK FSSLSLKALR RIVPLMQSGL | |
| | RYDEAVAQIP EYGHHSQRIE PGAAKHLYLP PFYEAQRKYA GKGDHIGSMQ | |
| | FRDDADIPRN PVVLRALNQA RKVVNALIRE YGSPIAVNIE MARDLSRPLD | |
| | ERNKVKRAQE EFRDRNDRAR SEFERDFGYK PKAAAFEKWM LYREQLGQCA | |
| | YSQQPLDIQR VLDDHNYAQV DHALPYSRSY DDSKNNKVLV LTHENQNKGN | |
| | RTAFEYLTSF PDGEDGERWR TFVAWVQGNK AYRMAKRNRL LRKNYGVDES | |
| | KGFIDRNLND TRYICKFFKN YVEEHLQLAA RADGDTARRC VVVNGQLTAF | |
| | LRARWGLTKV RGDSDRHHAL DAAVVAACTH GMVKALADYS RRKEISFLQE | |
| | GFPDPETGEI LNPAAFDRAR QHFPEPWTHF AHELKARLFT DDLAALREDM | |
| | QRLGSYTTED LGRLRTLFVS RAPQRRSGGA VHKETIYAQP ESLKQQGGVI | |
| | EKILLTSLKL QDFDKLLNPE SNDHFVEPHR NERLYAAIRQ RLEQFGGRAD | |
| | KAFGPDNLFH KPDKNNQPTG PVVRSIKLVR GKQTGIPIRG GLAKNDSMLR | |
| | VDIFTKAGKF HLVPVYVHHR VTGLPNRAIV AFKDEDEWTL IDESFAFLFS | |
| | VYPNDYVKVT LKKEQQSGYY SGADRSTGAM NLWAHDRAAS VGKDGLIRGI | |
| | GVKTALSVEK FNVDVLGRIY LAPPETRSGL A | |
| Porphyromonas | MLMSKHVLGL DLGVGSIGWC LIALDAQGDP AEILGMGSRV VPLNNATKAI | (SEQ |
| sp. oral | EAFNAGAAFT ASQERTARRT MRRGFARYQL RRYRLRRELE KVGMLPDAAL | ID |
| taxon 279 | IQLPLLELWE LRERAATAGR RLTLPELGRV LCHINQKRGY RHVKSDAAAI | NO: |
| str. F0450 | VGDEGEKKKD SNSAYLAGIR ANDEKLQAEH KTVGQYFAEQ LRQNQSESPT | 73) |
| WP_009433518.1 | GGISYRIKDQ IFSRQCYIDE YDQIMAVQRV HYPDILTDEF IRMLRDEVIF | |
| | MQRPLKSCKH LVSLCEFEKQ ERVMRVQQDD GKGGWQLVER RVKFGPKVAP | |
| | KSSPLFQLCC IYEAVNNIRL TRPNGSPCDI TPEERAKIVA HLQSSASLSF | |
| | AALKKLLKEK ALIADQLTSK SGLKGNSTRV ALASALQPYP QYHHLLDMEL | |
| | ETRMMTVQLT DEETGEVTER EVAVVIDSYV RKPLYRLWHI LYSIEEREAM | |
| | RRALITQLGM KEEDLDGGLL DQLYRLDFVK PGYGNKSAKF ICKLLPQLQQ | |
| | GLGYSEACAA VGYRHSNSPT SEEITERTLL EKIPLLQRNE LRQPLVEKIL | |
| | NQMINLVNAL KAEYGIDEVR VELARELKMS REERERMARN NKDREERNKG | |
| | VAAKIRECGL YPTKPRIQKY MLWKEAGRQC LYCGRSIEEE QCLREGGMEV | |
| | EHIIPKSVLY DDSYGNKTCA CRRCNKEKGN RTALEYIRAK GREAEYMKRI | |
| | NDLLKEKKIS YSKHQRLRWL KEDIPSDFLE RQLRLTQYIS RQAMAILQQG | |
| | IRRVSASEGG VTARLRSLWG YGKILHTLNL DRYDSMGETE RVSREGEATE | |
| | ELHITNWSKR MDHRHHAIDA LVVACTRQSY IQRLNRLSSE FGREDKKKED | |
| | QEAQEQQATE TGRLSNLERW LTQRPHESVR TVSDKVAEIL ISYRPGQRVV | |
| | TRGRNIYRKK MADGREVSCV QRGVLVPRGE LMEASFYGKI LSQGRVRIVK | |
| | RYPLHDLKGE VVDPHLRELI TTYNQELKSR EKGAPIPPLC LDKDKKQEVR | |
| | SVRCYAKTLS LDKAIPMCFD EKGEPTAFVK SASNHHLALY RTPKGKLVES | |
| | IVTFWDAVDR ARYGIPLVIT HPREVMEQVL QRGDIPEQVL SLLPPSDWVF | |
| | VDSLQQDEMV VIGLSDEELQ RALEAQNYRK ISEHLYRVQK MSSSYYVERY | |
| | HLETSVADDK NTSGRIPKFH RVQSLKAYEE RNIRKVRVDL LGRISLL | |
| Mycoplasma | MHNKKNITIG FDLGIASIGW AIIDSTTSKI LDWGTRTFEE RKTANERRAF | (SEQ |
| ovipneumoniae | RSTRRNIRRK AYRNQRFINL ILKYKDLFEL KNISDIQRAN KKDTENYEKI | ID |
| SC01 | ISFFTEIYKK CAAKHSNILE VKVKALDSKI EKLDLIWILH DYLENRGFFY | NO: |
| WP_010320922.1 | DLEEENVADK YEGIEHPSIL LYDFFKKNGF FKSNSSIPKD LGGYSFSNLQ | 74) |
| | WVNEIKKLFE VQEINPEFSE KFLNLFTSVR DYAKGPGSEH SASEYGIFQK | |
| | DEKGKVFKKY DNIWDKTIGK CSFFVEENRS PVNYPSYEIF NLLNQLINLS | |
| | TDLKTINKKI WQLSSNDRNE LLDELLKVKE KAKIISISLK KNEIKKIILK | |
| | DFGFEKSDID DQDTIEGRKI IKEEPTTKLE VTKHLLATIY SHSSDSNWIN | |
| | INNILEFLPY LDAICIILDR EKSRGQDEVL KLTEKNIFE VLKIDREKQL | |
| | DFVKSIFSNT KFNFKKIGNF SLKAIREFLP KMFEQNKNSE YLKWKDEEIR | |
| | RKWEEQKSKL GKTDKKTKYL NPRIFQDEII SPGTKNTFEQ AVLVLNQIIK | |
| | KYSKENIIDA IIIESPREKN DKKTIEEIKK RNKKGKGKTL EKLFQILNLE | |
| | NKGYKLSDLE TKPAKLLDRL RFYHQQDGID LYTLDKINID QLINGSQKYE | |
| | IEHIIPYSMS YDNSQANKIL TEKAENLKKG KLIASEYIKR NGDEFYNKYY | |
| | EKAKELFINK YKKNKKLDSY VDLDEDSAKN RFRFLTLQDY DEFQVEFLAR | |
| | NLNDTRYSTK LFYHALVEHF ENNEFFTYID ENSSKHKVKI STIKGHVTKY | |
| | FRAKPVQKNN GPNENLNNNK PEKIEKNREN NEHHAVDAAI VAIIGNKNPQ | |
| | IANLLTLADN KTDKKELLHD ENYKENIETG ELVKIPKFEV DKLAKVEDLK | |
| | KIIQEKYEEA KKHTAIKFSR KTRTILNGGL SDETLYGFKY DEKEDKYFKI | |
| | IKKKLVTSKN EELKKYFENP FGKKADGKSE YTVLMAQSHL SEFNKLKEIF | |
| | EKYNGFSNKT GNAFVEYMND LALKEPTLKA EIESAKSVEK LLYYNFKPSD | |
| | QFTYHDNINN KSFKRFYKNI RIIEYKSIPI KFKILSKHDG GKSFKDTLFS | |
| | LYSLVYKVYE NGKESYKSIP VISQMRNFGI DEFDELDENL YNKEKLDIYK | |
| | SDFAKPIPVN CKPVFVLKKG SILKKKSLDI DDFKETKETE EGNYYFISTI | |
| | SKRENRDTAY GLKPLKLSVV KPVAEPSTNP IFKEYIPIHL DELGNEYPVK | |
| | IKEHTDDEKL MCTIK | |
| Wolinella | MLVSPISVDL GGKNTGFFSF TDSLDNSQSG TVIYDESFVL SQVGRRSKRH | (SEQ |
| succinogenes | SKRNNLRNKL VKRLFLLILQ EHHGLSIDVL PDEIRGLENK RGYTYAGFEL | ID |
| WP_011139431.1 | DEKKKDALES DTLKEFLSEK LQSIDRDSDV EDFLNQIASN AESFKDYKKG | NO: |
| | FEAVFASATH SPNKKLELKD ELKSEYGENA KELLAGLRVT KEILDEFDKQ | 75) |
| | ENQGNLPRAK YFEELGEYIA TNEKVKSFFD SNSLKLTDMT KLIGNISNYQ | |
| | LKELRRYEND KEMEKGDIWI PNKLHKITER FVRSWHPKND ADRQRRAELM | |
| | KDLKSKEIME LLTTTEPVMT IPPYDDMNNR GAVKCQTLRL NEEYLDKHLP | |
| | NWRDIAKRLN HGKENDDLAD STVKGYSEDS TLLHRLLDTS KEIDIYELRG | |
| | KKPNELLVKT LGQSDANRLY GFAQNYYELI RQKVRAGIWV PVKNKDDSLN | |
| | LEDNSNMLKR CNHNPPHKKN QIHNLVAGIL GVKLDEAKFA EFEKELWSAK | |
| | VGNKKLSAYC KNIEELRKTH GNTFKIDIEE LRKKDPAELS KEEKAKLRLT | |
| | DDVILNEWSQ KIANFFDIDD KHRQRFNNLF SMAQLHTVID TPRSGFSSTC | |
| | KRCTAENRFR SETAFYNDET GEFHKKATAT CQRLPADTQR PFSGKIERYI | |
| | DKLGYELAKI KAKELEGMEA KEIKVPIILE QNAFEYEESL RKSKTGSNDR | |
| | VINSKKDRDG KKLAKAKENA EDRLKDKDKR IKAFSSGICP YCGDTIGDDG | |
| | EIDHILPRSH TLKIYGTVEN PEGNLIYVHQ KCNQAKADSI YKLSDIKAGV | |
| | SAQWIEEQVA NIKGYKTFSV LSAEQQKAFR YALFLQNDNE AYKKVVDWLR | |
| | TDQSARVNGT QKYLAKKIQE KLTKMLPNKH LSFEFILADA TEVSELRRQY | |
| | ARQNPLLAKA EKQAPSSHAI DAVMAFVARY QKVFKDGTPP NADEVAKLAM | |
| | LDSWNPASNE PLTKGLSTNQ KIEKMIKSGD YGQKNMREVE GKSIFGENAI | |
| | GERYKPIVVQ EGGYYIGYPA TVKKGYELKN CKVVTSKNDI AKLEKIIKNQ | |
| | DLISLKENQY IKIFSINKQT ISELSNRYFN MNYKNLVERD KEIVGLLEFI | |
| | VENCRYYTKK VDVKFAPKYI HETKYPFYDD WRRFDEAWRY LQENQNKTSS | |
| | KDRFVIDKSS LNEYYQPDKN EYKLDVDTQP IWDDFCRWYF LDRYKTANDK | |
| | KSIRIKARKT FSLLAESGVQ GKVFRAKRKI PTGYAYQALP MDNNVIAGDY | |
| | ANILLEANSK TLSLVPKSGI SIEKQLDKKL DVIKKTDVRG LAIDNNSFFN | |
| | ADFDTHGIRL IVENTSVKVG NFPISAIDKS AKRMIFRALF EKEKGKRKKK | |
| | TTISFKESGP VQDYLKVFLK KIVKIQLRTD GSISNIVVRK NAADFTLSER | |
| | SEHIQKLLK | |
| Streptococcus | MKKPYSIGLD IGTNSVGWAV VTDDYKVPAK KMKVLGNTDK SHIEKNLLGA | (SEQ |
| mutans | LLFDSGNTAE DRRLKRTARR RYTRRRNRIL YLQEIFSEEM GKVDDSFFHR | ID |
| UA159 | LEDSFLVTED KRGERHPIFG NLEEEVKYHE NFPTIYHLRQ YLADNPEKVD | NO: |
| WP_002263549.1 | LRLVYLALAH IIKFRGHFLI EGKFDTRNND VQRLFQEFLA VYDNTFENSS | 76) |
| | LQEQNVQVEE ILTDKISKSA KKDRVLKLFP NEKSNGRFAE FLKLIVGNQA | |
| | DFKKHFELEE KAPLQFSKDT YEEELEVLLA QIGDNYAELF LSAKKLYDSI | |
| | LLSGILTVTD VGTKAPLSAS MIQRYNEHQM DLAQLKQFIR QKLSDKYNEV | |
| | FSDVSKDGYA GYIDGKTNQE AFYKYLKGLL NKIEGSGYFL DKIEREDFLR | |
| | KQRTFDNGSI PHQIHLQEMR AIIRRQAEFY PFLADNQDRI EKLLTFRIPY | |
| | YVGPLARGKS DFAWLSRKSA DKITPWNFDE IVDKESSAEA FINRMTNYDL | |
| | YLPNQKVLPK HSLLYEKFTV YNELTKVKYK TEQGKTAFFD ANMKQEIFDG | |
| | VFKVYRKVTK DKLMDFLEKE FDEFRIVDLT GLDKENKVEN ASYGTYHDLC | |
| | KILDKDFLDN SKNEKILEDI VLTLTLFEDR EMIRKRLENY SDLLTKEQVK | |
| | KLERRHYTGW GRLSAELIHG IRNKESRKTI LDYLIDDGNS NRNEMQLIND | |
| | DALSFKEEIA KAQVIGETDN LNQVVSDIAG SPAIKKGILQ SLKIVDELVK | |
| | IMGHQPENIV VEMARENQFT NQGRRNSQQR LKGLTDSIKE FGSQILKEHP | |
| | VENSQLQNDR LFLYYLQNGR DMYTGEELDI DYLSQYDIDH IIPQAFIKDN | |
| | SIDNRVLTSS KENRGKSDDV PSKDVVRKMK SYWSKLLSAK LITQRKEDNL | |
| | TKAERGGLTD DDKAGFIKRQ LVETRQITKH VARILDEREN TETDENNKKI | |
| | RQVKIVILKS NLVSNERKEF ELYKVREIND YHHAHDAYLN AVIGKALLGV | |
| | YPQLEPEFVY GDYPHFHGHK ENKATAKKFF YSNIMNFFKK DDVRTDKNGE | |
| | IIWKKDEHIS NIKKVLSYPQ VNIVKKVEEQ TGGFSKESIL PKGNSDKLIP | |
| | RKTKKFYWDT KKYGGFDSPI VAYSILVIAD IEKGKSKKLK TVKALVGVTI | |
| | MEKMTFERDP VAFLERKGYR NVQEENIIKL PKYSLFKLEN GRKRLLASAR | |
| | ELQKGNEIVL PNHLGTLLYH AKNIHKVDEP KHLDYVDKHK DEFKELLDVV | |
| | SNFSKKYTLA EGNLEKIKEL YAQNNGEDLK ELASSFINLL TFTAIGAPAT | |
| | FKFEDKNIDR KRYTSTTEIL NATLIHQSIT GLYETRIDLN KLGGD | |
| Prevotella | MNKRILGLDT GTNSLGWAVV DWDEHAQSYE LIKYGDVIFQ EGVKIEKGIE | (SEQ |
| timonensis | SSKAAERSGY KAIRKQYFRR RLRKIQVLKV LVKYHLCPYL SDDDLRQWHL | ID |
| CRIS 5C- | QKQYPKSDEL MLWQRTSDEE GKNPYYDRHR CLHEKLDLTV EADRYTLGRA | NO: |
| B1 | LYHLTQRRGF LSNRLDTSAD NKEDGVVKSG ISQLSTEMEE AGCEYLGDYF | 77) |
| WP_008122718.1 | YKLYDAQGNK VRIRQRYTDR NKHYQHEFDA ICEKQELSSE LIEDLQRAIF | |
| | FQLPLKSQRH GVGRCTFERG KPRCADSHPD YEEFRMLCFV NNIQVKGPHD | |
| | LELRPLTYEE REKIEPLFFR KSKPNFDFED IAKALAGKKN YAWIHDKEER | |
| | AYKFNYRMTQ GVPGCPTIAQ LKSIFGDDWK TGIAETYTLI QKKNGSKSLQ | |
| | EMVDDVWNVL YSFSSVEKLK EFAHHKLQLD EESAEKFAKI KLSHSFAALS | |
| | LKAIRKFLPF LRKGMYYTHA SFFANIPTIV GKEIWNKEQN RKYIMENVGE | |
| | LVFNYQPKHR EVQGTIEMLI KDFLANNFEL PAGATDKLYH PSMIETYPNA | |
| | QRNEFGILQL GSPRINAIRN PMAMRSLHIL RRVVNQLLKE SIIDENTEVH | |
| | VEYARELNDA NKRRAIADRQ KEQDKQHKKY GDEIRKLYKE ETGKDIEPTQ | |
| | TDVLKFQLWE EQNHHCLYTG EQIGITDFIG SNPKFDIEHT IPQSVGGDST | |
| | QMNLTLCDNR FNREVKKAKL PTELANHEEI LTRIEPWKNK YEQLVKERDK | |
| | QRTFAGMDKA VKDIRIQKRH KLQMEIDYWR GKYERFTMTE VPEGFSRRQG | |
| | TGIGLISRYA GLYLKSLFHQ ADSRNKSNVY VVKGVATAEF RKMWGLQSEY | |
| | EKKCRDNHSH HCMDAITIAC IGKREYDLMA EYYRMEETFK QGRGSKPKFS | |
| | KPWATFTEDV LNIYKNLLVV HDTPNNMPKH TKKYVQTSIG KVLAQGDTAR | |
| | GSLHLDTYYG AIERDGEIRY VVRRPLSSFT KPEELENIVD ETVKRTIKEA | |
| | IADKNFKQAI AEPIYMNEEK GILIKKVRCF AKSVKQPINI RQHRDLSKKE | |
| | YKQQYHVMNE NNYLLAIYEG LVKNKVVREF EIVSYIEAAK YYKRSQDRNI | |
| | FSSIVPTHST KYGLPLKTKL LMGQLVLMFE ENPDEIQVDN TKDLVKRLYK | |
| | VVGIEKDGRI KFKYHQEARK EGLPIFSTPY KNNDDYAPIF RQSINNINIL | |
| | VDGIDFTIDI LGKVTLKE | |
| Clostridium | MKYTLGLDVG IASVGWAVID KDNNKIIDLG VRCFDKAEES KTGESLATAR | (SEQ |
| cellulolyticum | RIARGMRRRI SRRSQRLRLV KKLFVQYEII KDSSEFNRIF DTSRDGWKDP | ID |
| H10 | WELRYNALSR ILKPYELVQV LTHITKRRGF KSNRKEDLST TKEGVVITSI | NO: |
| ACL77411.1 | KNNSEMLRTK NYRTIGEMIF METPENSNKR NKVDEYIHTI AREDLLNEIK | 78) |
| | YIFSIQRKLG SPFVTEKLEH DELNIWEFQR PFASGDSILS KVGKCTLLKE | |
| | ELRAPTSCYT SEYFGLLQSI NNLVLVEDNN TLTLNNDQRA KIIEYAHFKN | |
| | EIKYSEIRKL LDIEPEILFK AHNLTHKNPS GNNESKKFYE MKSYHKLKST | |
| | LPTDIWGKLH SNKESLDNLF YCLTVYKNDN EIKDYLQANN LDYLIEYIAK | |
| | LPTFNKFKHL SLVAMKRIIP FMEKGYKYSD ACNMAELDFT GSSKLEKCNK | |
| | LTVEPIIENV TNPVVIRALT QARKVINAII QKYGLPYMVN IELAREAGMT | |
| | RQDRDNLKKE HENNRKAREK ISDLIRQNGR VASGLDILKW RLWEDQGGRC | |
| | AYSGKPIPVC DLLNDSLTQI DHIYPYSRSM DDSYMNKVLV LTDENQNKRS | |
| | YTPYEVWGST EKWEDFEARI YSMHLPQSKE KRLLNRNFIT KDLDSFISRN | |
| | LNDTRYISRF LKNYIESYLQ FSNDSPKSCV VCVNGQCTAQ LRSRWGLNKN | |
| | REESDLHHAL DAAVIACADR KIIKEITNYY NERENHNYKV KYPLPWHSFR | |
| | QDLMETLAGV FISRAPRRKI TGPAHDETIR SPKHFNKGLT SVKIPLTTVT | |
| | LEKLETMVKN TKGGISDKAV YNVLKNRLIE HNNKPLKAFA EKIYKPLKNG | |
| | TNGAIIRSIR VETPSYTGVF RNEGKGISDN SLMVRVDVFK KKDKYYLVPI | |
| | YVAHMIKKEL PSKAIVPLKP ESQWELIDST HEFLFSLYQN DYLVIKTKKG | |
| | ITEGYYRSCH RGTGSLSLMP HFANNKNVKI DIGVRTAISI EKYNVDILGN | |
| | KSIVKGEPRR GMEKYNSFKS N | |
| Francisella | MNFKILPIAI DLGVKNTGVF SAFYQKGTSL ERLDNKNGKV YELSKDSYTL | (SEQ |
| tularensis | LMNNRTARRH QRRGIDRKQL VKRLFKLIWT EQLNLEWDKD TQQAISFLEN | ID |
| subsp. | RRGFSFITDG YSPEYLNIVP EQVKAILMDI FDDYNGEDDL DSYLKLATEQ | NO: |
| novicida | ESKISEIYNK LMQKILEFKL MKLCTDIKDD KVSTKTLKEI TSYEFELLAD | 79) |
| U112 | YLANYSESLK TQKFSYTDKQ GNLKELSYYH HDKYNIQEFL KRHATINDRI | |
| WP_003038941.1 | LDTLLTDDLD IWNFNFEKED FDKNEEKLQN QEDKDHIQAH LHHFVFAVNK | |
| | IKSEMASGGR HRSQYFQEIT NVLDENNHQE GYLKNFCENL HNKKYSNLSV | |
| | KNLVNLIGNL SNLELKPLRK YFNDKIHAKA DHWDEQKFTE TYCHWILGEW | |
| | RVGVKDQDKK DGAKYSYKDL CNELKQKVTK AGLVDELLEL DPCRTIPPYL | |
| | DNNNRKPPKC QSLILNPKFL DNQYPNWQQY LQELKKLQSI QNYLDSFETD | |
| | LKVLKSSKDQ PYFVEYKSSN QQIASGQRDY KDLDARILQF IFDRVKASDE | |
| | LLLNEIYFQA KKLKQKASSE LEKLESSKKL DEVIANSQLS QILKSQHING | |
| | IFEQGTFLHL VCKYYKQRQR ARDSRLYIMP EYRYDKKLHK YNNTGRFDDD | |
| | NQLLTYCNHK PRQKRYQLLN DLAGVLQVSP NFLKDKIGSD DDLFISKWLV | |
| | EHIRGFKKAC EDSLKIQKDN RGLLNHKINI ARNTKGKCEK EIFNLICKIE | |
| | GSEDKKGNYK HGLAYELGVL LFGEPNEASK PEFDRKIKKF NSIYSFAQIQ | |
| | QIAFAERKGN ANTCAVCSAD NAHRMQQIKI TEPVEDNKDK IILSAKAQRL | |
| | PAIPTRIVDG AVKKMATILA KNIVDDNWQN IKQVLSAKHQ LHIPIITESN | |
| | AFEFEPALAD VKGKSLKDRR KKALERISPE NIFKDKNNRI KEFAKGISAY | |
| | SGANLTDGDF DGAKEELDHI IPRSHKKYGT LNDEANLICV TRGDNKNKGN | |
| | RIFCLRDLAD NYKLKQFETT DDLEIEKKIA DTIWDANKKD FKFGNYRSFI | |
| | NLTPQEQKAF RHALFLADEN PIKQAVIRAI NNRNRTFVNG TQRYFAEVLA | |
| | NNIYLRAKKE NLNTDKISFD YFGIPTIGNG RGIAEIRQLY EKVDSDIQAY | |
| | AKGDKPQASY SHLIDAMLAF CIAADEHRND GSIGLEIDKN YSLYPLDKNT | |
| | GEVFTKDIFS QIKITDNEFS DKKLVRKKAI EGENTHRQMT RDGIYAENYL | |
| | PILIHKELNE VRKGYTWKNS EEIKIFKGKK YDIQQLNNLV YCLKFVDKPI | |
| | SIDIQISTLE ELRNILTINN IAATAEYYYI NLKTQKLHEY YIENYNTALG | |
| | YKKYSKEMEF LRSLAYRSER VKIKSIDDVK QVLDKDSNFI IGKITLPFKK | |
| | EWQRLYREWQ NTTIKDDYEF LKSFFNVKSI TKLHKKVRKD FSLPISTNEG | |
| | KFLVKRKTWD NNFIYQILND SDSRADGTKP FIPAFDISKN EIVEAIIDSF | |
| | TSKNIFWLPK NIELQKVDNK NIFAIDTSKW FEVETPSDLR DIGIATIQYK | |
| | IDNNSRPKVR VKLDYVIDDD SKINYFMNHS LLKSRYPDKV LEILKQSTII | |
| | EFESSGFNKT IKEMLGMKLA GIYNETSNN | |
| Azospirillum | MARPAFRAPR REHVNGWTPD PHRISKPFFI LVSWHLLSRV VIDSSSGCFP | (SEQ |
| sp. B510 | GTSRDHTDKF AEWECAVQPY RLSFDLGINS IGWGLLNLDR QGKPREIRAL | ID |
| AOL40891.1 | GSRIFSDGRD PQDKASLAVA RRLARQMRRR RDRYLTRRTR LMGALVRFGL | NO: |
| | MPADPAARKR LEVAVDPYLA RERATRERLE PFEIGRALFH LNQRRGYKPV | 80) |
| | RTATKPDEEA GKVKEAVERL EAAIAAAGAP TLGAWFAWRK TRGETLRARL | |
| | AGKGKEAAYP FYPARRMLEA EFDTLWAEQA RHHPDLLTAE AREILRHRIF | |
| | HQRPLKPPPV GRCTLYPDDG RAPRALPSAQ RLRLFQELAS LRVIHLDLSE | |
| | RPLTPAERDR IVAFVQGRPP KAGRKPGKVQ KSVPFEKLRG LLELPPGTGF | |
| | SLESDKRPEL LGDETGARIA PAFGPGWTAL PLEEQDALVE LLLTEAEPER | |
| | AIAALTARWA LDEATAAKLA GATLPDFHGR YGRRAVAELL PVLERETRGD | |
| | PDGRVRPIRL DEAVKLLRGG KDHSDFSREG ALLDALPYYG AVLERHVAFG | |
| | TGNPADPEEK RVGRVANPTV HIALNQLRHL VNAILARHGR PEEIVIELAR | |
| | DLKRSAEDRR REDKRQADNQ KRNEERKRLI LSLGERPTPR NLLKLRLWEE | |
| | QGPVENRRCP YSGETISMRM LLSEQVDIDH ILPFSVSLDD SAANKVVCLR | |
| | EANRIKRNRS PWEAFGHDSE RWAGILARAE ALPKNKRWRF APDALEKLEG | |
| | EGGLRARHLN DTRHLSRLAV EYLRCVCPKV RVSPGRLTAL LRRRWGIDAI | |
| | LAEADGPPPE VPAETLDPSP AEKNRADHRH HALDAVVIGC IDRSMVQRVQ | |
| | LAAASAEREA AAREDNIRRV LEGFKEEPWD GFRAELERRA RTIVVSHRPE | |
| | HGIGGALHKE TAYGPVDPPE EGENLVVRKP IDGLSKDEIN SVRDPRLRRA | |
| | LIDRLAIRRR DANDPATALA KAAEDLAAQP ASRGIRRVRV LKKESNPIRV | |
| | EHGGNPSGPR SGGPFHKLLL AGEVHHVDVA LRADGRRWVG HWVTLFEAHG | |
| | GRGADGAAAP PRLGDGERFL MRLHKGDCLK LEHKGRVRVM QVVKLEPSSN | |
| | SVVVVEPHQV KTDRSKHVKI SCDQLRARGA RRVTVDPLGR VRVHAPGARV | |
| | GIGGDAGRTA MEPAEDIS | |
| Peptoniphilus | MKNLKEYYIG LDIGTASVGW AVTDESYNIP KFNGKKMWGV RLFDDAKTAE | (SEQ |
| duerdenii | ERRTQRGSRR RLNRRKERIN LLQDLFATEI SKVDPNFFLR LDNSDLYRED | ID |
| ATCC | KDEKLKSKYT LENDKDFKDR DYHKKYPTIH HLIMDLIEDE GKKDIRLLYL | NO: |
| BAA-1640 | ACHYLLKNRG HFIFEGQKED TKNSFDKSIN DLKIHLRDEY NIDLEFNNED | 81) |
| WP_008901059.1 | LIEIITDTTL NKTNKKKELK NIVGDTKFLK AISAIMIGSS QKLVDLFEDG | |
| | EFEETTVKSV DESTTAFDDK YSEYEEALGD TISLLNILKS IYDSSILENL | |
| | LKDADKSKDG NKYISKAFVK KFNKHGKDLK TLKRIIKKYL PSEYANIFRN | |
| | KSINDNYVAY TKSNITSNKR TKASKFTKQE DFYKFIKKHL DTIKETKLNS | |
| | SENEDLKLID EMLTDIEFKT FIPKLKSSDN GVIPYQLKLM ELKKILDNQS | |
| | KYYDFLNESD EYGTVKDKVE SIMEFRIPYY VGPLNPDSKY AWIKRENTKI | |
| | TPWNFKDIVD LDSSREEFID RLIGRCTYLK EEKVLPKASL IYNEFMVLNE | |
| | LNNLKLNEFL ITEEMKKAIF EELFKTKKKV TLKAVSNLLK KEFNLTGDIL | |
| | LSGTDGDFKQ GLNSYIDFKN IIGDKVDRDD YRIKIEEIIK LIVLYEDDKT | |
| | YLKKKIKSAY KNDFTDDEIK KIAALNYKDW GRLSKRFLTG IEGVDKTTGE | |
| | KGSIIYFMRE YNLNLMELMS GHYTFTEEVE KLNPVENREL CYEMVDELYL | |
| | SPSVKRMLWQ SLRVVDEIKR IIGKDPKKIF IEMARAKEAK NSRKESRKNK | |
| | LLEFYKFGKK AFINEIGEER YNYLLNEINS EEESKFRWDN LYLYYTQLGR | |
| | CMYSLEPIDL ADLKSNNIYD QDHIYPKSKI YDDSLENRVL VKKNLNHEKG | |
| | NQYPIPEKVL NKNAYGFWKI LFDKGLIGQK KYTRLTRRTP FEERELAEFI | |
| | ERQIVETRQA TKETANLLKN ICQDSEIVYS KAENASRFRQ EFDIIKCRTV | |
| | NDLHHMHDAY LNIVVGNVYN TKFTKNPLNF IKDKDNVRSY NLENMFKYDV | |
| | VRGSYTAWIA DDSEGNVKAA TIKKVKRELE GKNYRFTRMS YIGTGGLYDQ | |
| | NLMRKGKGQI PQKENTNKSN IEKYGGYNKA SSAYFALIES DGKAGRERTL | |
| | ETIPIMVYNQ EKYGNTEAVD KYLKDNLELQ DPKILKDKIK INSLIKLDGF | |
| | LYNIKGKTGD SLSIAGSVQL IVNKEEQKLI KKMDKFLVKK KDNKDIKVTS | |
| | FDNIKEEELI KLYKTLSDKL NNGIYSNKRN NQAKNISEAL DKFKEISIEE | |
| | KIDVLNQIIL LFQSYNNGCN LKSIGLSAKT GVVFIPKKLN YKECKLINQS | |
| | ITGLFENEVD LLNL | |
| Lactobacillus | MGYRIGLDVG ITSTGYAVLK TDKNGLPYKI LTLDSVIYPR AENPQTGASL | (SEQ |
| coryniformis | AEPRRIKRGL RRRTRRTKFR KQRTQQLFIH SGLLSKPEIE QILATPQAKY | ID |
| subsp. | SVYELRVAGL DRRLTNSELF RVLYFFIGHR GFKSNRKAEL NPENEADKKQ | NO: |
| torquens | MGQLLNSIEE IRKAIAEKGY RTVGELYLKD PKYNDHKRNK GYIDGYLSTP | 82) |
| KCTC 3535 | NRQMLVDEIK QILDKQRELG NEKLTDEFYA TYLLGDENRA GIFQAQRDFD | |
| WP_010014406.1 | EGPGAGPYAG DQIKKMVGKD IFEPTEDRAA KATYTFQYEN LLQKMTSLNY | |
| | QNTTGDTWHT LNGLDRQAII DAVFAKAEKP TKTYKPTDFG ELRKLLKLPD | |
| | DARFNLVNYG SLQTQKEIET VEKKTRFVDF KAYHDLVKVL PEEMWQSRQL | |
| | LDHIGTALTL YSSDKRRRRY FAEELNLPAE LIEKLLPLNF SKFGHLSIKS | |
| | MQNIIPYLEM GQVYSEATTN TGYDERKKQI SKDTIREEIT NPVVRRAVTK | |
| | TIKIVEQIIR RYGKPDGINI ELARELGRNF KERGDIQKRQ DKNRQTNDKI | |
| | AAELTELGIP VNGQNIIRYK LHKEQNGVDP YTGDQIPFER AFSEGYEVDH | |
| | IIPYSISWDD SYTNKVLTSA KCNREKGNRI PMVYLANNEQ RLNALTNIAD | |
| | NIIRNSRKRQ KLLKQKLSDE ELKDWKQRNI NDTRFITRVL YNYFRQAIEF | |
| | NPELEKKQRV LPLNGEVTSK IRSRWGFLKV REDGDLHHAI DATVIAAITP | |
| | KFIQQVTKYS QHQEVKNNQA LWHDAEIKDA EYAAEAQRMD ADLENKIFNG | |
| | FPLPWPEFLD ELLARISDNP VEMMKSRSWN TYTPIEIAKL KPVFVVRLAN | |
| | HKISGPAHLD TIRSAKLFDE KGIVLSRVSI TKLKINKKGQ VATGDGIYDP | |
| | ENSNNGDKVV YSAIRQALEA HNGSGELAFP DGYLEYVDHG TKKLVRKVRV | |
| | AKKVSLPVRL KNKAAADNGS MVRIDVENTG KKFVFVPIYI KDTVEQVLPN | |
| | KAIARGKSLW YQITESDQFC FSLYPGDMVH IESKTGIKPK YSNKENNTSV | |
| | VPIKNFYGYF DGADIATASI LVRAHDSSYT ARSIGIAGLL KFEKYQVDYF | |
| | GRYHKVHEKK RQLFVKRDE | |
| Ignavibacterium | MEFKKVLGLD IGINSIGCAL LSLPKSIQDY GKGGRLEWLT SRVIPLDADY | (SEQ |
| album | MKAFIDGKNG LPQVITPAGK RRQKRGSRRL KHRYKLRRSR LIRVEKTLNW | ID |
| JCM 16511 | LPEDFPLDNP KRIKETISTE GKFSFRISDY VPISDESYRE FYREFGYPEN | NO: |
| WP_014561873.1 | EIEQVIEEIN FRRKTKGKNK NPMIKLLPED WVVYYLRKKA LIKPTTKEEL | 83) |
| | IRIIYLENQR RGFKSSRKDL TETAILDYDE FAKRLAEKEK YSAENYETKF | |
| | VSITKVKEVV ELKTDGRKGK KRFKVILEDS RIEPYEIERK EKPDWEGKEY | |
| | TFLVTQKLEK GKFKQNKPDL PKEEDWALCT TALDNRMGSK HPGEFFFDEL | |
| | LKAFKEKRGY KIRQYPVNRW RYKKELEFIW TKQCQLNPEL NNLNINKEIL | |
| | RKLATVLYPS QSKFFGPKIK EFENSDVLHI ISEDIIYYQR DLKSQKSLIS | |
| | ECRYEKRKGI DGEIYGLKCI PKSSPLYQEF RIWQDIHNIK VIRKESEVNG | |
| | KKKINIDETQ LYINENIKEK LFELFNSKDS LSEKDILELI SLNIINSGIK | |
| | ISKKEEETTH RINLFANRKE LKGNETKSRY RKVFKKLGFD GEYILNHPSK | |
| | LNRLWHSDYS NDYADKEKTE KSILSSLGWK NRNGKWEKSK NYDVENLPLE | |
| | VAKAIANLPP LKKEYGSYSA LAIRKMLVVM RDGKYWQHPD QIAKDQENTS | |
| | LMLFDKNLIQ LTNNQRKVLN KYLLTLAEVQ KRSTLIKQKL NEIEHNPYKL | |
| | ELVSDQDLEK QVLKSFLEKK NESDYLKGLK TYQAGYLIYG KHSEKDVPIV | |
| | NSPDELGEYI RKKLPNNSLR NPIVEQVIRE TIFIVRDVWK SFGIIDEIHI | |
| | ELGRELKNNS EERKKTSESQ EKNFQEKERA RKLLKELLNS SNFEHYDENG | |
| | NKIFSSFTVN PNPDSPLDIE KFRIWKNQSG LTDEELNKKL KDEKIPTEIE | |
| | VKKYILWLTQ KCRSPYTGKI IPLSKLFDSN VYEIEHIIPR SKMKNDSTNN | |
| | LVICELGVNK AKGDRLAANF ISESNGKCKF GEVEYTLLKY GDYLQYCKDT | |
| | FKYQKAKYKN LLATEPPEDF IERQINDTRY IGRKLAELLT PVVKDSKNII | |
| | FTIGSITSEL KITWGLNGVW KDILRPRFKR LESIINKKLI FQDEDDPNKY | |
| | HFDLSINPQL DKEGLKRLDH RHHALDATII AATTREHVRY LNSLNAADND | |
| | EEKREYFLSL CNHKIRDFKL PWENFTSEVK SKLLSCVVSY KESKPILSDP | |
| | FNKYLKWEYK NGKWQKVFAI QIKNDRWKAV RRSMFKEPIG TVWIKKIKEV | |
| | SLKEAIKIQA IWEEVKNDPV RKKKEKYIYD DYAQKVIAKI VQELGLSSSM | |
| | RKQDDEKLNK FINEAKVSAG VNKNLNTINK TIYNLEGRFY EKIKVAEYVL | |
| | YKAKRMPLNK KEYIEKLSLQ KMENDLPNFI LEKSILDNYP EILKELESDN | |
| | KYIIEPHKKN NPVNRLLLEH ILEYHNNPKE AFSTEGLEKL NKKAINKIGK | |
| | PIKYITRLDG DINEEEIFRG AVFETDKGSN VYFVMYENNQ TKDREFLKPN | |
| | PSISVLKAIE HKNKIDFFAP NRLGFSRIIL SPGDLVYVPT NDQYVLIKDN | |
| | SSNETIINWD DNEFISNRIY QVKKFTGNSC YFLKNDIASL ILSYSASNGV | |
| | GEFGSQNISE YSVDDPPIRI KDVCIKIRVD RLGNVRPL | |
| uncultured | MSSKAIDSLE QLDLFKPQEY TLGLDLGIKS IGWAILSGER IANAGVYLFE | (SEQ |
| delta | TAEELNSTGN KLISKAAERG RKRRIRRMLD RKARRGRHIR YLLEREGLPT | ID |
| proteobacterium | DELEEVVVHQ SNRTLWDVRA EAVERKLTKQ ELAAVLFHLV RHRGYFPNTK | NO: |
| HF0070_07 | KLPPDDESDS ADEEQGKINR ATSRLREELK ASDCKTIGQF LAQNRDRQRN | 84) |
| E19 | REGDYSNLMA RKLVFEEALQ ILAFQRKQGH ELSKDFEKTY LDVLMGQRSG | |
| ADI19058.1 | RSPKLGNCSL IPSELRAPSS APSTEWFKFL QNLGNLQISN AYREEWSIDA | |
| | PRRAQIIDAC SQRSTSSYWQ IRRDFQIPDE YRENLVNYER RDPDVDLQEY | |
| | LQQQERKTLA NERNWKQLEK IIGTGHPIQT LDEAARLITL IKDDEKLSDQ | |
| | LADLLPEASD KAITQLCELD FTTAAKISLE AMYRILPHMN QGMGFFDACQ | |
| | QESLPEIGVP PAGDRVPPED EMYNPVVNRV LSQSRKLINA VIDEYGMPAK | |
| | IRVELARDLG KGRELRERIK LDQLDKSKQN DQRAEDFRAE FQQAPRGDQS | |
| | LRYRLWKEQN CTCPYSGRMI PVNSVLSEDT QIDHILPISQ SFDNSLSNKV | |
| | LCFTEENAQK SNRTPFEYLD AADFQRLEAI SGNWPEAKRN KLLHKSFGKV | |
| | AEEWKSRALN DTRYLTSALA DHLRHHLPDS KIQTVNGRIT GYLRKQWGLE | |
| | KDRDKHTHHA VDAIVVACTT PAIVQQVTLY HQDIRRYKKL GEKRPTPWPE | |
| | TFRQDVLDVE EEIFITRQPK KVSGGIQTKD TLRKHRSKPD RQRVALTKVK | |
| | LADLERLVEK DASNRNLYEH LKQCLEESGD QPTKAFKAPF YMPSGPEAKQ | |
| | RPILSKVILL REKPEPPKQL TELSGGRRYD SMAQGRLDIY RYKPGGKRKD | |
| | EYRVVLQRMI DLMRGEENVH VFQKGVPYDQ GPEIEQNYTF LFSLYFDDLV | |
| | EFQRSADSEV IRGYYRTFNI ANGQLKISTY LEGRQDFDFF GANRLAHFAK | |
| | VQVNLLGKVI K | |
| Ruminococcus | MGNYYLGLDV GIGSIGWAVI NIEKKRIEDF NVRIFKSGEI QEKNRNSRAS | (SEQ |
| albus 8 | QQCRRSRGLR RLYRRKSHRK LRLKNYLSII GLTTSEKIDY YYETADNNVI | ID |
| WP_002846926.1 | QLRNKGLSEK LTPEEIAACL IHICNNRGYK DFYEVNVEDI EDPDERNEYK | NO: |
| | EEHDSIVLIS NLMNEGGYCT PAEMICNCRE FDEPNSVYRK FHNSAASKNH | 85) |
| | YLITRHMLVK EVDLILENQS KYYGILDDKT IAKIKDIIFA QRDFEIGPGK | |
| | NERFRRFTGY LDSIGKCQFF KDQERGSRFT VIADIYAFVN VLSQYTYTNN | |
| | RGESVFDTSF ANDLINSALK NGSMDKRELK AIAKSYHIDI SDKNSDTSLT | |
| | KCFKYIKVVK PLFEKYGYDW DKLIENYTDT DNNVLNRIGI VLSQAQTPKR | |
| | RREKLKALNI GLDDGLINEL TKLKLSGTAN VSYKYMQGSI EAFCEGDLYG | |
| | KYQAKFNKEI PDIDENAKPQ KLPPFKNEDD CEFFKNPVVF RSINETRKLI | |
| | NAIIDKYGYP AAVNIETADE LNKTFEDRAI DTKRNNDNQK ENDRIVKEII | |
| | ECIKCDEVHA RHLIEKYKLW EAQEGKCLYS GETITKEDML RDKDKLFEVD | |
| | HIVPYSLILD NTINNKALVY AEENQKKGQR TPLMYMNEAQ AADYRVRVNT | |
| | MFKSKKCSKK KYQYLMLPDL NDQELLGGWR SRNLNDTRYI CKYLVNYLRK | |
| | NLRFDRSYES SDEDDLKIRD HYRVFPVKSR FTSMFRRWWL NEKTWGRYDK | |
| | AELKKLTYLD HAADAIIIAN CRPEYVVLAG EKLKLNKMYH QAGKRITPEY | |
| | EQSKKACIDN LYKLFRMDRR TAEKLLSGHG RLTPIIPNLS EEVDKRLWDK | |
| | NIYEQFWKDD KDKKSCEELY RENVASLYKG DPKFASSLSM PVISLKPDHK | |
| | YRGTITGEEA IRVKEIDGKL IKLKRKSISE ITAESINSIY TDDKILIDSL | |
| | KTIFEQADYK DVGDYLKKIN QHFFTTSSGK RVNKVTVIEK VPSRWLRKEI | |
| | DDNNFSLLND SSYYCIELYK DSKGDNNLQG IAMSDIVHDR KTKKLYLKPD | |
| | FNYPDDYYTH VMYIFPGDYL RIKSTSKKSG EQLKFEGYFI SVKNVNENSF | |
| | RFISDNKPCA KDKRVSITKK DIVIKLAVDL MGKVQGENNG KGISCGEPLS | |
| | LLKEKN | |
| Lactobacillus | MTKKEQPYNI GLDIGTSSVG WAVINDNYDL LNIKKKNLWG VRLFEEAQTA | (SEQ |
| farciminis | KETRINRSTR RRYRRRKNRI NWLNEIFSEE LAKTDPSFLI RLQNSWVSKK | ID |
| KCTC 3681 | DPDRKRDKYN LFIDGPYTDK EYYREFPTIF HLRKELILNK DKADIRLIYL | NO: |
| WP_010018949.1 | ALHNILKYRG NFTYEHQKEN ISNLNNNLSK ELIELNQQLI KYDISFPDDC | 86) |
| | DWNHISDILI GRGNATQKSS NILKDFTLDK ETKKLLKEVI NLILGNVAHL | |
| | NTIFKTSLTK DEEKLNFSGK DIESKLDDLD SILDDDQFTV LDAANRIYST | |
| | ITLNEILNGE SYFSMAKVNQ YENHAIDLCK LRDMWHTTKN EEAVEQSRQA | |
| | YDDYINKPKY GTKELYTSLK KFLKVALPTN LAKEAEEKIS KGTYLVKPRN | |
| | SENGVVPYQL NKIEMEKIID NQSQYYPFLK ENKEKLLSIL SFRIPYYVGP | |
| | LQSAEKNPFA WMERKSNGHA RPWNFDEIVD REKSSNKFIR RMTVTDSYLV | |
| | GEPVLPKNSL IYQRYEVLNE LNNIRITENL KTNPIGSRLT VETKQRIYNE | |
| | LFKKYKKVTV KKLTKWLIAQ GYYKNPILIG LSQKDEFNST LTTYLDMKKI | |
| | FGSSFMEDNK NYDQIEELIE WLTIFEDKQI LNEKLHSSKY SYTPDQIKKI | |
| | SNMRYKGWGR LSKKILMDIT TETNTPQLLQ LSNYSILDLM WATNNNFISI | |
| | MSNDKYDFKN YIENHNLNKN EDQNISDLVN DIHVSPALKR GITQSIKIVQ | |
| | EIVKFMGHAP KHIFIEVTRE TKKSEITTSR EKRIKRLQSK LLNKANDFKP | |
| | QLREYLVPNK KIQEELKKHK NDLSSERIML YFLQNGKSLY SEESLNINKL | |
| | SDYQVDHILP RTYIPDDSLE NKALVLAKEN QRKADDLLLN SNVIDRNLER | |
| | WTYMLNNNMI GLKKFKNLTR RVITDKDKLG FIHRQLVQTS QMVKGVANIL | |
| | DNMYKNQGTT CIQARANLST AFRKALSGQD DTYHFKHPEL VKNRNVNDFH | |
| | HAQDAYLASF LGTYRLRRFP TNEMLLMNGE YNKFYGQVKE LYSKKKKLPD | |
| | SRKNGFIISP LVNGTTQYDR NTGEIIWNVG FRDKILKIFN YHQCNVTRKT | |
| | EIKTGQFYDQ TIYSPKNPKY KKLIAQKKDM DPNIYGGFSG DNKSSITIVK | |
| | IDNNKIKPVA IPIRLINDLK DKKTLQNWLE ENVKHKKSIQ IIKNNVPIGQ | |
| | IIYSKKVGLL SLNSDREVAN RQQLILPPEH SALLRLLQIP DEDLDQILAF | |
| | YDKNILVEIL QELITKMKKF YPFYKGEREF LIANIENENQ ATTSEKVNSL | |
| | EELITLLHAN STSAHLIFNN IEKKAFGRKT HGLTLNNTDF IYQSVTGLYE | |
| | TRIHIE | |
| Eubacterium | MMEVFMGRLV LGLDIGITSV GFGIIDLDES EIVDYGVRLF KEGTAAENET | (SEQ |
| dolichum | RRTKRGGRRL KRRRVTRRED MLHLLKQAGI ISTSFHPLNN PYDVRVKGLN | ID |
| DSM 3991 | ERLNGEELAT ALLHLCKHRG SSVETIEDDE AKAKEAGETK KVLSMNDQLL | NO: |
| WP_004800457.1 | KSGKYVCEIQ KERLRTNGHI RGHENNEKTR AYVDEAFQIL SHQDLSNELK | 87) |
| | SAIITIISRK RMYYDGPGGP LSPTPYGRYT YFGQKEPIDL IEKMRGKCSL | |
| | FPNEPRAPKL AYSAELENLL NDLNNLSIEG EKLTSEQKAM ILKIVHEKGK | |
| | ITPKQLAKEV GVSLEQIRGF RIDTKGSPLL SELTGYKMIR EVLEKSNDEH | |
| | LEDHVFYDEI AEILTKTKDI EGRKKQISEL SSDLNEESVH QLAGLTKFTA | |
| | YHSLSFKALR LINEEMLKTE LNQMQSITLF GLKQNNELSV KGMKNIQADD | |
| | TAILSPVAKR AQRETFKVVN RLREIYGEFD SIVVEMAREK NSEEQRKAIR | |
| | ERQKFFEMRN KQVADIIGDD RKINAKLREK LVLYQEQDGK TAYSLEPIDL | |
| | KLLIDDPNAY EVDHIIPISI SLDDSITNKV LVTHRENQEK GNLTPISAFV | |
| | KGRFTKGSLA QYKAYCLKLK EKNIKTNKGY RKKVEQYLLN ENDIYKYDIQ | |
| | KEFINRNLVD TSYASRVVLN TLTTYFKQNE IPTKVFTVKG SLTNAFRRKI | |
| | NLKKDRDEDY GHHAIDALII ASMPKMRLLS TIFSRYKIED IYDESTGEVF | |
| | SSGDDSMYYD DRYFAFIASL KAIKVRKFSH KIDTKPNRSV ADETIYSTRV | |
| | IDGKEKVVKK YKDIYDPKFT ALAEDILNNA YQEKYLMALH DPQTFDQIVK | |
| | VVNYYFEEMS KSEKYFTKDK KGRIKISGMN PLSLYRDEHG MLKKYSKKGD | |
| | GPAITQMKYF DGVLGNHIDI SAHYQVRDKK VVLQQISPYR TDFYYSKENG | |
| | YKFVTIRYKD VRWSEKKKKY VIDQQDYAMK KAEKKIDDTY EFQFSMHRDE | |
| | LIGITKAEGE ALIYPDETWH NFNFFFHAGE TPEILKFTAT NNDKSNKIEV | |
| | KPIHCYCKMR LMPTISKKIV RIDKYATDVV GNLYKVKKNT LKFEFD | |
| Nitratifractor | MKKILGVDLG ITSFGYAILQ ETGKDLYRCL DNSVVMRNNP YDEKSGESSQ | (SEQ |
| salsuginis | SIRSTQKSMR RLIEKRKKRI RCVAQTMERY GILDYSETMK INDPKNNPIK | ID |
| DSM 16511 | NRWQLRAVDA WKRPLSPQEL FAIFAHMAKH RGYKSIATED LIYELELELG | NO: |
| ADV46720.1 | LNDPEKESEK KADERRQVYN ALRHLEELRK KYGGETIAQT IHRAVEAGDL | 88) |
| | RSYRNHDDYE KMIRREDIEE EIEKVLLRQA ELGALGLPEE QVSELIDELK | |
| | ACITDQEMPT IDESLFGKCT FYKDELAAPA YSYLYDLYRL YKKLADLNID | |
| | GYEVTQEDRE KVIEWVEKKI AQGKNLKKIT HKDLRKILGL APEQKIFGVE | |
| | DERIVKGKKE PRTFVPFFFL ADIAKFKELI ASIQKHPDAL QIFRELAEIL | |
| | QRSKTPQEAL DRLRALMAGK GIDTDDRELL ELFKNKRSGT RELSHRYILE | |
| | ALPLFLEGYD EKEVQRILGF DDREDYSRYP KSLRHLHLRE GNLFEKEENP | |
| | INNHAVKSLA SWALGLIADL SWRYGPFDEI ILETTRDALP EKIRKEIDKA | |
| | MREREKALDK IIGKYKKEFP SIDKRLARKI QLWERQKGLD LYSGKVINLS | |
| | QLLDGSADIE HIVPQSLGGL STDYNTIVTL KSVNAAKGNR LPGDWLAGNP | |
| | DYRERIGMLS EKGLIDWKKR KNLLAQSLDE IYTENTHSKG IRATSYLEAL | |
| | VAQVLKRYYP FPDPELRKNG IGVRMIPGKV TSKTRSLLGI KSKSRETNFH | |
| | HAEDALILST LTRGWQNRLH RMLRDNYGKS EAELKELWKK YMPHIEGLTL | |
| | ADYIDEAFRR FMSKGEESLF YRDMEDTIRS ISYWVDKKPL SASSHKETVY | |
| | SSRHEVPTLR KNILEAFDSL NVIKDRHKLT TEEFMKRYDK EIRQKLWLHR | |
| | IGNTNDESYR AVEERATQIA QILTRYQLMD AQNDKEIDEK FQQALKELIT | |
| | SPIEVTGKLL RKMRFVYDKL NAMQIDRGLV ETDKNMLGIH ISKGPNEKLI | |
| | FRRMDVNNAH ELQKERSGIL CYLNEMLFIF NKKGLIHYGC LRSYLEKGQG | |
| | SKYIALFNPR FPANPKAQPS KFTSDSKIKQ VGIGSATGII KAHLDLDGHV | |
| | RSYEVFGTLP EGSIEWFKEE SGYGRVEDDP HH | |
| Rhodospirillum | MRPIEPWILG LDIGTDSLGW AVFSCEEKGP PTAKELLGGG VRLFDSGRDA | (SEQ |
| rubrum | KDHTSRQAER GAFRRARRQT RTWPWRRDRL IALFQAAGLT PPAAETRQIA | ID |
| ATCC | LALRREAVSR PLAPDALWAA LLHLAHHRGF RSNRIDKRER AAAKALAKAK | NO: |
| 11170 | PAKATAKATA PAKEADDEAG FWEGAEAALR QRMAASGAPT VGALLADDLD | 89) |
| WP_011388212.1 | RGQPVRMRYN QSDRDGVVAP TRALIAEELA EIVARQSSAY PGLDWPAVTR | |
| | LVLDQRPLRS KGAGPCAFLP GEDRALRALP TVQDFIIRQT LANLRLPSTS | |
| | ADEPRPLTDE EHAKALALLS TARFVEWPAL RRALGLKRGV KFTAETERNG | |
| | AKQAARGTAG NLTEAILAPL IPGWSGWDLD RKDRVFSDLW AARQDRSALL | |
| | ALIGDPRGPT RVTEDETAEA VADAIQIVLP TGRASLSAKA ARAIAQAMAP | |
| | GIGYDEAVTL ALGLHHSHRP RQERLARLPY YAAALPDVGL DGDPVGPPPA | |
| | EDDGAAAEAY YGRIGNISVH IALNETRKIV NALLHRHGPI LRLVMVETTR | |
| | ELKAGADERK RMIAEQAERE RENAEIDVEL RKSDRWMANA RERRQRVRLA | |
| | RRQNNLCPYT STPIGHADLL GDAYDIDHVI PLARGGRDSL DNMVLCQSDA | |
| | NKTKGDKTPW EAFHDKPGWI AQRDDFLARL DPQTAKALAW RFADDAGERV | |
| | ARKSAEDEDQ GFLPRQLTDT GYIARVALRY LSLVTNEPNA VVATNGRLTG | |
| | LLRLAWDITP GPAPRDLLPT PRDALRDDTA ARRFLDGLTP PPLAKAVEGA | |
| | VQARLAALGR SRVADAGLAD ALGLTLASLG GGGKNRADHR HHFIDAAMIA | |
| | VTTRGLINQI NQASGAGRIL DLRKWPRINF EPPYPTFRAE VMKQWDHIHP | |
| | SIRPAHRDGG SLHAATVFGV RNRPDARVLV QRKPVEKLFL DANAKPLPAD | |
| | KIAEIIDGFA SPRMAKRFKA LLARYQAAHP EVPPALAALA VARDPAFGPR | |
| | GMTANTVIAG RSDGDGEDAG LITPFRANPK AAVRTMGNAV YEVWEIQVKG | |
| | RPRWTHRVLT RFDRTQPAPP PPPENARLVM RLRRGDLVYW PLESGDRLFL | |
| | VKKMAVDGRL ALWPARLATG KATALYAQLS CPNINLNGDQ GYCVQSAEGI | |
| | RKEKIRTTSC TALGRLRLSK KAT | |
| Finegoldia | MKSEKKYYIG LDVGTNSVGW AVTDEFYNIL RAKGKDLWGV RLFEKADTAA | (SEQ |
| magna | NTRIFRSGRR RNDRKGMRLQ ILREIFEDEI KKVDKDFYDR LDESKFWAED | ID |
| ATCC | KKVSGKYSLF NDKNFSDKQY FEKFPTIFHL RKYLMEEHGK VDIRYYFLAI | NO: |
| 29328 | NQMMKRRGHF LIDGQISHVT DDKPLKEQLI LLINDLLKIE LEEELMDSIF | 90) |
| WP_012290141.1 | EILADVNEKR TDKKNNLKEL IKGQDENKQE GNILNSIFES IVTGKAKIKN | |
| | IISDEDILEK IKEDNKEDFV LTGDSYEENL QYFEEVLQEN ITLFNTLKST | |
| | YDFLILQSIL KGKSTLSDAQ VERYDEHKKD LEILKKVIKK YDEDGKLFKQ | |
| | VFKEDNGNGY VSYIGYYLNK NKKITAKKKI SNIEFTKYVK GILEKQCDCE | |
| | DEDVKYLLGK IEQENFLLKQ ISSINSVIPH QIHLFELDKI LENLAKNYPS | |
| | FNNKKEEFTK IEKIRKTFTF RIPYYVGPLN DYHKNNGGNA WIFRNKGEKI | |
| | RPWNFEKIVD LHKSEEEFIK RMLNQCTYLP EETVLPKSSI LYSEYMVLNE | |
| | LNNLRINGKP LDTDVKLKLI EELFKKKTKV TLKSIRDYMV RNNFADKEDF | |
| | DNSEKNLEIA SNMKSYIDEN NILEDKEDVE MVEDLIEKIT IHTGNKKLLK | |
| | KYIEETYPDL SSSQIQKIIN LKYKDWGRLS RKLLDGIKGT KKETEKTDTV | |
| | INFLRNSSDN LMQIIGSQNY SFNEYIDKLR KKYIPQEISY EVVENLYVSP | |
| | SVKKMIWQVI RVTEEITKVM GYDPDKIFIE MAKSEEEKKT TISRKNKLLD | |
| | LYKAIKKDER DSQYEKLLTG LNKLDDSDLR SRKLYLYYTQ MGRDMYTGEK | |
| | IDLDKLFDST HYDKDHIIPQ SMKKDDSIIN NLVLVNKNAN QTTKGNIYPV | |
| | PSSIRNNPKI YNYWKYLMEK EFISKEKYNR LIRNTPLTNE ELGGFINRQL | |
| | VETRQSTKAI KELFEKFYQK SKIIPVKASL ASDLRKDMNT LKSREVNDLH | |
| | HAHDAFLNIV AGDVWNREFT SNPINYVKEN REGDKVKYSL SKDFTRPRKS | |
| | KGKVIWTPEK GRKLIVDTLN KPSVLISNES HVKKGELFNA TIAGKKDYKK | |
| | GKIYLPLKKD DRLQDVSKYG GYKAINGAFF FLVEHTKSKK RIRSIELFPL | |
| | HLLSKFYEDK NTVLDYAINV LQLQDPKIII DKINYRTEII IDNESYLIST | |
| | KSNDGSITVK PNEQMYWRVD EISNLKKIEN KYKKDAILTE EDRKIMESYI | |
| | DKIYQQFKAG KYKNRRTTDT IIEKYEIIDL DTLDNKQLYQ LLVAFISLSY | |
| | KTSNNAVDFT VIGLGTECGK PRITNLPDNT YLVYKSITGI YEKRIRIK | |
| Eubacterium | MNYTEKEKLF MKYILALDIG IASVGWAILD KESETVIEAG SNIFPEASAA | (SEQ |
| rectale | DNQLRRDMRG AKRNNRRLKT RINDFIKLWE NNNLSIPQFK STEIVGLKVR | ID |
| ATCC | AITEEITLDE LYLILYSYLK HRGISYLEDA LDDTVSGSSA YANGLKLNAK | NO: |
| 33656 | ELETHYPCEI QQERLNTIGK YRGQSQIINE NGEVLDLSNV FTIGAYRKEI | 91) |
| WP_012742555.1 | QRVFEIQKKY HPELTDEFCD GYMLIFNRKR KYYEGPGNEK SRTDYGRFTT | |
| | KLDANGNYIT EDNIFEKLIG KCSVYPDELR AAAASYTAQE YNVLNDLNNL | |
| | TINGRKLEEN EKHEIVERIK SSNTINMRKI ISDCMGENID DFAGARIDKS | |
| | GKEIFHKFEV YNKMRKALLE IGIDISNYSR EELDEIGYIM TINTDKEAMM | |
| | EAFQKSWIDL SDDVKQCLIN MRKTNGALEN KWQSFSLKIM NELIPEMYAQ | |
| | PKEQMTLLTE MGVTKGTQEE FAGLKYIPVD VVSEDIFNPV VRRSVRISFK | |
| | ILNAVLKKYK ALDTIVIEMP RDRNSEEQKK RINDSQKLNE KEMEYIEKKL | |
| | AVTYGIKLSP SDFSSQKQLS LKLKLWNEQD GICLYSGKTI DPNDIINNPQ | |
| | LFEIDHIIPR SISEDDARSN KVLVYRSENQ KKGNQTPYYY LTHSHSEWSF | |
| | EQYKATVMNL SKKKEYAISR KKIQNLLYSE DITKMDVLKG FINRNINDTS | |
| | YASRLVLNTI QNFFMANEAD TKVKVIKGSY THQMRCNLKL DKNRDESYSH | |
| | HAVDAMLIGY SELGYEAYHK LQGEFIDFET GEILRKDMWD ENMSDEVYAD | |
| | YLYGKKWANI RNEVVKAEKN VKYWHYVMRK SNRGLCNQTI RGTREYDGKQ | |
| | YKINKLDIRT KEGIKVFAKL AFSKKDSDRE RLLVYLNDRR TFDDLCKIYE | |
| | DYSDAANPFV QYEKETGDII RKYSKKHNGP RIDKLKYKDG EVGACIDISH | |
| | KYGFEKGSKK VILESLVPYR MDVYYKEENH SYYLVGVKQS DIKFEKGRNV | |
| | IDEEAYARIL VNEKMIQPGQ SRADLENLGF KFKLSFYKND IIEYEKDGKI | |
| | YTERLVSRTM PKQRNYIETK PIDKAKFEKQ NLVGLGKTKF IKKYRYDILG | |
| | NKYSCSEEKF TSFC | |
| Corynebacterium | MKYHVGIDVG TFSVGLAAIE VDDAGMPIKT LSLVSHIHDS GLDPDKIKSA | (SEQ |
| diphtheriae | VTRLASSGIA RRTRRLYRRK RRRLQQLDKF IQRQGWPVIE LEDYSDPLYP | ID |
| C7 (beta) | WKVRAELAAS YIADEKERGE KLSVALRHIA RHRGWRNPYA KVSSLYLPDE | NO: |
| AEX66236.1 | PSDAFKAIRE EIKRASGQPV PETATVGQMV TLCELGTLKL RGEGGVLSAR | 92) |
| WP_014318431.1 | LQQSDHAREI QEICRMQEIG QELYRKIIDV VFAAESPKGS ASSRVGKDPL | |
| | QPGKNRALKA SDAFQRYRIA ALIGNLRVRV DGEKRILSVE EKNLVEDHLV | |
| | NLAPKKEPEW VTIAEILGID RGQLIGTATM TDDGERAGAR PPTHDTNRSI | |
| | VNSRIAPLVD WWKTASALEQ HAMVKALSNA EVDDFDSPEG AKVQAFFADL | |
| | DDDVHAKLDS LHLPVGRAAY SEDTLVRLTR RMLADGVDLY TARLQEFGIE | |
| | PSWTPPAPRI GEPVGNPAVD RVLKTVSRWL ESATKTWGAP ERVIIEHVRE | |
| | GFVTEKRARE MDGDMRRRAA RNAKLFQEMQ EKLNVQGKPS RADLWRYQSV | |
| | QRQNCQCAYC GSPITFSNSE MDHIVPRAGQ GSTNTRENLV AVCHRCNQSK | |
| | GNTPFAIWAK NTSIEGVSVK EAVERTRHWV TDTGMRSTDF KKFTKAVVER | |
| | FQRATMDEEI DARSMESVAW MANELRSRVA QHFASHGTTV RVYRGSLTAE | |
| | ARRASGISGK LEFLDGVGKS RLDRRHHAID AAVIAFTSDY VAETLAVRSN | |
| | LKQSQAHRQE APQWREFTGK DAEHRAAWRV WCQKMEKLSA LLTEDLRDDR | |
| | VVVMSNVRLR LGNGSAHEET IGKLSKVKLG SQLSVSDIDK ASSEALWCAL | |
| | TREPDFDPKD GLPANPERHI RVNGTHVYAG DNIGLFPVSA GSIALRGGYA | |
| | ELGSSFHHAR VYKITSGKKP AFAMLRVYTI DLLPYRNQDL FSVELKPQTM | |
| | SMRQAEKKLR DALATGNAEY LGWLVVDDEL VVDTSKIATD QVKAVEAELG | |
| | TIRRWRVDGF FGDTRLRLRP LQMSKEGIKK ESAPELSKII DRPGWLPAVN | |
| | KLFSEGNVTV VRRDSLGRVR LESTAHLPVT WKVQ | |
| Roseburia | MNAEHGKEGL LIMEENFQYR IGLDIGITSV GWAVLQNNSQ DEPVRITDLG | (SEQ |
| inulinivorans | VRIFDVAENP KNGDALAAPR RDARTTRRRL RRRRHRLERI KFLLQENGLI | ID |
| DSM | EMDSFMERYY KGNLPDVYQL RYEGLDRKLK DEELAQVLIH IAKHRGERST | NO: |
| 16841 | RKAETKEKEG GAVLKATTEN QKIMQEKGYR TVGEMLYLDE AFHTECLWNE | 93) |
| WP_007889305.1 | KGYVLTPRNR PDDYKHTILR SMLVEEVHAI FAAQRAHGNQ KATEGLEEAY | |
| | VEIMTSQRSF DMGPGLQPDG KPSPYAMEGF GDRVGKCTFE KDEYRAPKAT | |
| | YTAELFVALQ KINHTKLIDE FGTGRFFSEE ERKTIIGLLL SSKELKYGTI | |
| | RKKLNIDPSL KENSLNYSAK KEGETEEERV LDTEKAKFAS MFWTYEYSKC | |
| | LKDRTEEMPV GEKADLFDRI GEILTAYKND DSRSSRLKEL GLSGEEIDGL | |
| | LDLSPAKYQR VSLKAMRKMQ PYLEDGLIYD KACEAAGYDF RALNDGNKKH | |
| | LLKGEEINAI VNDITNPVVK RSVSQTIKVI NAIIQKYGSP QAVNIELARE | |
| | MSKNFQDRIN LEKEMKKRQQ ENERAKQQII ELGKQNPTGQ DILKYRLWND | |
| | QGGYCLYSGK KIPLEELFDG GYDIDHILPY SITEDDSYRN KVLVTAQENR | |
| | QKGNRTPYEY FGADEKRWED YEASVRLLVR DYKKQQKLLK KNFTEEERKE | |
| | FKERNLNDTK YITRVVYNMI RQNLELEPEN HPEKKKQVWA VNGAVTSYLR | |
| | KRWGLMQKDR STDRHHAMDA VVIACCTDGM IHKISRYMQG RELAYSRNFK | |
| | FPDEETGEIL NRDNFTREQW DEKFGVKVPL PWNSERDELD IRLLNEDPKN | |
| | FLLTHADVQR ELDYPGWMYG EEESPIEEGR YINYIRPLFV SRMPNHKVTG | |
| | SAHDATIRSA RDYETRGVVI TKVPLTDLKL NKDNEIEGYY DKDSDRLLYQ | |
| | ALVRQLLLHG NDGKKAFAED FHKPKADGTE GPVVRKVKIE KKQTSGVMVR | |
| | GGTGIAANGE MVRIDVFREN GKYYFVPVYT ADVVRKVLPN RAATHTKPYS | |
| | EWRVMDDANF VFSLYSRDLI HVKSKKDIKT NLVNGGLLLQ KEIFAYYTGA | |
| | DIATASIAGF ANDSNFKFRG LGIQSLEIFE KCQVDILGNI SVVRHENRQE | |
| | FH | |
| Alicycliphilus | MRSLRYRLAL DLGSTSLGWA LFRLDACNRP TAVIKAGVRI FSDGRNPKDG | (SEQ |
| denitrificans | SSLAVTRRAA RAMRRRRDRL LKRKTRMQAK LVEHGFFPAD AGKRKALEQL | ID |
| K601 | NPYALRAKGL QEALLPGEFA RALFHINQRR GFKSNRKTDK KDNDSGVLKK | NO: |
| WP_013517127.1 | AIGQLRQQMA EQGSRTVGEY LWTRLQQGQG VRARYREKPY TTEEGKKRID | 94) |
| | KSYDLYIDRA MIEQEFDALW AAQAAFNPTL FHEAARADLK DTLLHQRPLR | |
| | PVKPGRCTLL PEEERAPLAL PSTQRFRIHQ EVNHLRLLDE NLREVALTLA | |
| | QRDAVVTALE TKAKLSFEQI RKLLKLSGSV QFNLEDAKRT ELKGNATSAA | |
| | LARKELFGAA WSGFDEALQD EIVWQLVTEE GEGALIAWLQ THTGVDEARA | |
| | QAIVDVSLPE GYGNLSRKAL ARIVPALRAA VITYDKAVQA AGFDHHSQLG | |
| | FEYDASEVED LVHPETGEIR SVFKQLPYYG KALQRHVAFG SGKPEDPDEK | |
| | RYGKIANPTV HIGLNQVRMV VNALIRRYGR PTEVVIELAR DLKQSREQKV | |
| | EAQRRQADNQ RRNARIRRSI AEVLGIGEER VRGSDIQKWI CWEELSFDAA | |
| | DRRCPYSGVQ ISAAMLLSDE VEVEHILPFS KTLDDSLNNR TVAMRQANRI | |
| | KRNRTPWDAR AEFEAQGWSY EDILQRAERM PLRKRYRFAP DGYERWLGDD | |
| | KDFLARALND TRYLSRVAAE YLRLVCPGTR VIPGQLTALL RGKFGLNDVL | |
| | GLDGEKNRND HRHHAVDACV IGVTDQGLMQ RFATASAQAR GDGLTRLVDG | |
| | MPMPWPTYRD HVERAVRHIW VSHRPDHGFE GAMMEETSYG IRKDGSIKQR | |
| | RKADGSAGRE ISNLIRIHEA TQPLRHGVSA DGQPLAYKGY VGGSNYCIEI | |
| | TVNDKGKWEG EVISTFRAYG VVRAGGMGRL RNPHEGQNGR KLIMRLVIGD | |
| | SVRLEVDGAE RTMRIVKISG SNGQIFMAPI HEANVDARNT DKQDAFTYTS | |
| | KYAGSLQKAK TRRVTISPIGEVRDPGFKG | |
| Sphaerochaeta | MSKKVSRRYE EQAQEICQRL GSRPYSIGLD LGVGSIGVAV AAYDPIKKQP | (SEQ |
| lobosa | SDLVFVSSRI FIPSTGAAER RQKRGQRNSL RHRANRLKFL WKLLAERNLM | ID |
| str. Buddy | LSYSEQDVPD PARLRFEDAV VRANPYELRL KGLNEQLTLS ELGYALYHIA | NO: |
| WP_013607849.1 | NHRGSSSVRT FLDEEKSSDD KKLEEQQAMT EQLAKEKGIS TFIEVLTAFN | 95) |
| | TNGLIGYRNS ESVKSKGVPV PTRDIISNEI DVLLQTQKQF YQEILSDEYC | |
| | DRIVSAILFE NEKIVPEAGC CPYFPDEKKL PRCHELNEER RLWEAINNAR | |
| | IKMPMQEGAA KRYQSASFSD EQRHILFHIA RSGTDITPKL VQKEFPALKT | |
| | SIIVLQGKEK AIQKIAGFRF RRLEEKSFWK RLSEEQKDDF FSAWTNTPDD | |
| | KRLSKYLMKH LLLTENEVVD ALKTVSLIGD YGPIGKTATQ LLMKHLEDGL | |
| | TYTEALERGM ETGEFQELSV WEQQSLLPYY GQILTGSTQA LMGKYWHSAF | |
| | KEKRDSEGFF KPNTNSDEEK YGRIANPVVH QTLNELRKLM NELITILGAK | |
| | PQEITVELAR ELKVGAEKRE DIIKQQTKQE KEAVLAYSKY CEPNNLDKRY | |
| | IERFRLLEDQ AFVCPYCLEH ISVADIAAGR ADVDHIFPRD DTADNSYGNK | |
| | VVAHRQCNDI KGKRTPYAAF SNTSAWGPIM HYLDETPGMW RKRRKFETNE | |
| | EEYAKYLQSK GFVSRFESDN SYIAKAAKEY LRCLENPNNV TAVGSLKGME | |
| | TSILRKAWNL QGIDDLLGSR HWSKDADTSP TMRKNRDDNR HHGLDAIVAL | |
| | YCSRSLVQMI NTMSEQGKRA VEIEAMIPIP GYASEPNLSF EAQRELFRKK | |
| | ILEFMDLHAF VSMKTDNDAN GALLKDTVYS ILGADTQGED LVFVVKKKIK | |
| | DIGVKIGDYE EVASAIRGRI TDKQPKWYPM EMKDKIEQLQ SKNEAALQKY | |
| | KESLVQAAAV LEESNRKLIE SGKKPIQLSE KTISKKALEL VGGYYYLISN | |
| | NKRTKTFVVK EPSNEVKGFA FDTGSNLCLD FYHDAQGKLC GEIIRKIQAM | |
| | NPSYKPAYMK QGYSLYVRLY QGDVCELRAS DLTEAESNLA KTTHVRLPNA | |
| | KPGRTFVIII TFTEMGSGYQ IYFSNLAKSK KGQDTSFTLT TIKNYDVRKV | |
| | QLSSAGLVRY VSPLLVDKIE KDEVALCGE | |
| Fusobacterium | MKKQKFSDYY LGFDIGTNSV GWCVTDLDYN VLRFNKKDMW GSRLFDEAKT | (SEQ |
| nucleatum | AAERRVQRNS RRRLKRRKWR LNLLEEIFSD EIMKIDSNFF RRLKESSLWL | ID |
| subsp. | EDKNSKEKFT LENDDNYKDY DFYKQYPTIF HLRDELIKNP EKKDIRLIYL | NO: |
| vincentii | ALHSIFKSRG HELFEGQNLK EIKNFETLYN NLISFLEDNG INKSIDKDNI | 96) |
| ATCC | EKLEKIICDS GKGLKDKEKE FKGIFNSDKQ LVAIFKLSVG SSVSLNDLED | |
| 49256 | TDEYKKEEVE KEKISFREQI YEDDKPIYYS ILGEKIELLD IAKSFYDEMV | |
| WP_005888649.1 | LNNILSDSNY ISEAKVKLYE EHKKDLKNLK YIIRKYNKEN YDKLFKDKNE | |
| | NNYPAYIGLN KEKDKKEVVE KSRLKIDDLI KVIKGYLPKP ERIEEKDKTI | |
| | FNEILNKIEL KTILPKQRIS DNGTLPYQIH EVELEKILEN QSKYYDELNY | |
| | EENGVSTKDK LLKTFKFRIP YYVGPLNSYH KDKGGNSWIV RKEEGKILPW | |
| | NFEQKVDIEK SAEEFIKRMT NKCTYLNGED VIPKDSFLYS EYIILNELNK | |
| | VQVNDEFLNE ENKRKIIDEL FKENKKVSEK KFKEYLLVNQ IANRTVELKG | |
| | IKDSFNSNYV SYIKFKDIFG EKLNLDIYKE ISEKSILWKC LYGDDKKIFE | |
| | KKIKNEYGDI LNKDEIKKIN SFKFNTWGRL SEKLLTGIEF INLETGECYS | |
| | SVMEALRRTN YNLMELLSSK FTLQESIDNE NKEMNEVSYR DLIEESYVSP | |
| | SLKRAILQTL KIYEEIKKIT GRVPKKVFIE MARGGDESMK NKKIPARQEQ | |
| | LKKLYDSCGN DIANFSIDIK EMKNSLSSYD NNSLRQKKLY LYYLQFGKCM | |
| | YTGREIDLDR LLQNNDTYDI DHIYPRSKVI KDDSFDNLVL VLKNENAEKS | |
| | NEYPVKKEIQ EKMKSFWRFL KEKNFISDEK YKRLTGKDDF ELRGFMARQL | |
| | VNVRQTTKEV GKILQQIEPE IKIVYSKAEI ASSFREMFDF IKVRELNDTH | |
| | HAKDAYLNIV AGNVYNTKFT EKPYRYLQEI KENYDVKKIY NYDIKNAWDK | |
| | ENSLEIVKKN MEKNTVNITR FIKEEKGELF NLNPIKKGET SNEIISIKPK | |
| | LYDGKDNKLN EKYGYYTSLK AAYFIYVEHE KKNKKVKTFE RITRIDSTLI | |
| | KNEKNLIKYL VSQKKLLNPK IIKKIYKEQT LIIDSYPYTF TGVDSNKKVE | |
| | LKNKKQLYLE KKYEQILKNA LKFVEDNQGE TEENYKFIYL KKRNNNEKNE | |
| | TIDAVKERYN IEFNEMYDKF LEKLSSKDYK NYINNKLYTN FLNSKEKFKK | |
| | LKLWEKSLIL REFLKIFNKN TYGKYEIKDS QTKEKLFSFP EDTGRIRLGQ | |
| | SSLGNNKELL EESVTGLFVK KIKL | |
| Pasteurella | MQTTNLSYIL GLDLGIASVG WAVVEINENE DPIGLIDVGV RIFERAEVPK | (SEQ |
| multocida | TGESLALSRR LARSTRRLIR RRAHRLLLAK RFLKREGILS TIDLEKGLPN | ID |
| subsp. | QAWELRVAGL ERRLSAIEWG AVLLHLIKHR GYLSKRKNES QTNNKELGAL | NO: |
| multocida | LSGVAQNHQL LQSDDYRTPA ELALKKFAKE EGHIRNQRGA YTHTFNRLDL | 97) |
| str. Pm70 | LAELNLLFAQ QHQFGNPHCK EHIQQYMTEL LMWQKPALSG EAILKMLGKC | |
| WP_010907033.1 | THEKNEFKAA KHTYSAERFV WLTKLNNLRI LEDGAERALN EEERQLLINH | |
| | PYEKSKLTYA QVRKLLGLSE QAIFKHLRYS KENAESATFM ELKAWHAIRK | |
| | ALENQGLKDT WQDLAKKPDL LDEIGTAFSL YKTDEDIQQY LINKVPNSVI | |
| | NALLVSLNED KFIELSLKSL RKILPLMEQG KRYDQACREI YGHHYGEANQ | |
| | KTSQLLPAIP AQEIRNPVVL RTLSQARKVI NAIIRQYGSP ARVHIETGRE | |
| | LGKSFKERRE IQKQQEDNRT KRESAVQKFK ELFSDESSEP KSKDILKERL | |
| | YEQQHGKCLY SGKEINIHRL NEKGYVEIDH ALPFSRTWDD SFNNKVLVLA | |
| | SENQNKGNQT PYEWLQGKIN SERWKNFVAL VLGSQCSAAK KQRLLTQVID | |
| | DNKFIDRNLN DTRYIARFLS NYIQENLLLV GKNKKNVFTP NGQITALLRS | |
| | RWGLIKAREN NNRHHALDAI VVACATPSMQ QKITRFIRFK EVHPYKIENR | |
| | YEMVDQESGE IISPHFPEPW AYFRQEVNIR VFDNHPDTVL KEMLPDRPQA | |
| | NHQFVQPLFV SRAPTRKMSG QGHMETIKSA KRLAEGISVL RIPLTQLKPN | |
| | LLENMVNKER EPALYAGLKA RLAEFNQDPA KAFATPFYKQ GGQQVKAIRV | |
| | EQVQKSGVLV RENNGVADNA SIVRTDVFIK NNKFFLVPIY TWQVAKGILP | |
| | NKAIVAHKNE DEWEEMDEGA KFKFSLFPND LVELKTKKEY FFGYYIGLDR | |
| | ATGNISLKEH DGEISKGKDG VYRVGVKLAL SFEKYQVDEL GKNRQICRPQ | |
| | QRQPVR | |
| Alcanivorax | MRYRVGLDLG TASVGAAVFS MDEQGNPMEL IWHYERLFSE PLVPDMGQLK | (SEQ |
| pacificus | PKKAARRLAR QQRRQIDRRA SRLRRIAIVS RRLGIAPGRN DSGVHGNDVP | ID |
| W11-5 | TLRAMAVNER IELGQLRAVL LRMGKKRGYG GTFKAVRKVG EAGEVASGAS | NO: |
| WP_008738269.1 | RLEEEMVALA SVQNKDSVTV GEYLAARVEH GLPSKLKVAA NNEYYAPEYA | 98) |
| | LFRQYLGLPA IKGRPDCLPN MYALRHQIEH EFERIWATQS QFHDVMKDHG | |
| | VKEEIRNAIF FQRPLKSPAD KVGRCSLQTN LPRAPRAQIA AQNFRIEKQM | |
| | ADLRWGMGRR AEMLNDHQKA VIRELLNQQK ELSFRKIYKE LERAGCPGPE | |
| | GKGLNMDRAA LGGRDDLSGN TTLAAWRKLG LEDRWQELDE VTQIQVINFL | |
| | ADLGSPEQLD TDDWSCRFMG KNGRPRNFSD EFVAFMNELR MTDGFDRLSK | |
| | MGFEGGRSSY SIKALKALTE WMIAPHWRET PETHRVDEEA AIRECYPESL | |
| | ATPAQGGRQS KLEPPPLTGN EVVDVALRQV RHTINMMIDD LGSVPAQIVV | |
| | EMAREMKGGV TRRNDIEKQN KRFASERKKA AQSIEENGKT PTPARILRYQ | |
| | LWIEQGHQCP YCESNISLEQ ALSGAYTNFE HILPRTLTQI GRKRSELVLA | |
| | HRECNDEKGN RTPYQAFGHD DRRWRIVEQR ANALPKKSSR KTRLLLLKDF | |
| | EGEALTDESI DEFADRQLHE SSWLAKVTTQ WLSSLGSDVY VSRGSLTAEL | |
| | RRRWGLDTVI PQVRFESGMP VVDEEGAEIT PEEFEKFRLQ WEGHRVTREM | |
| | RTDRRPDKRI DHRHHLVDAI VTALTSRSLY QQYAKAWKVA DEKQRHGRVD | |
| | VKVELPMPIL TIRDIALEAV RSVRISHKPD RYPDGRFFEA TAYGIAQRLD | |
| | ERSGEKVDWL VSRKSLTDLA PEKKSIDVDK VRANISRIVG EAIRLHISNI | |
| | FEKRVSKGMT PQQALREPIE FQGNILRKVR CFYSKADDCV RIEHSSRRGH | |
| | HYKMLLNDGF AYMEVPCKEG ILYGVPNLVR PSEAVGIKRA PESGDFIRFY | |
| | KGDTVKNIKT GRVYTIKQIL GDGGGKLILT PVTETKPADL LSAKWGRLKV | |
| | GGRNIHLLRL CAE | |
| Mycoplasma | MYFYKNKENK LNKKVVLGLD LGIASVGWCL TDISQKEDNK FPIILHGVRL | (SEQ |
| mobile | FETVDDSDDK LLNETRRKKR GQRRRNRRLF TRKRDFIKYL IDNNIIELEF | ID |
| 163K | DKNPKILVRN FIEKYINPFS KNLELKYKSV TNLPIGFHNL RKAAINEKYK | NO: |
| AAT27519.1 | LDKSELIVLL YFYLSLRGAF FDNPEDTKSK EMNKNEIEIF DKNESIKNAE | 99) |
| | FPIDKIIEFY KISGKIRSTI NLKFGHQDYL KEIKQVFEKQ NIDEMNYEKF | |
| | AMEEKSFFSR IRNYSEGPGN EKSFSKYGLY ANENGNPELI INEKGQKIYT | |
| | KIFKTLWESK IGKCSYDKKL YRAPKNSFSA KVEDITNKLT DWKHKNEYIS | |
| | ERLKRKILLS RFLNKDSKSA VEKILKEENI KFENLSEIAY NKDDNKINLP | |
| | IINAYHSLTT IFKKHLINFE NYLISNENDL SKLMSFYKQQ SEKLFVPNEK | |
| | GSYEINQNNN VLHIFDAISN ILNKFSTIQD RIRILEGYFE FSNLKKDVKS | |
| | SEIYSEIAKL REFSGTSSLS FGAYYKFIPN LISEGSKNYS TISYEEKALQ | |
| | NQKNNFSHSN LFEKTWVEDL IASPTVKRSL RQTMNLLKEI FKYSEKNNLE | |
| | IEKIVVEVTR SSNNKHERKK IEGINKYRKE KYEELKKVYD LPNENTTLLK | |
| | KLWLLRQQQG YDAYSLRKIE ANDVINKPWN YDIDHIVPRS ISFDDSESNL | |
| | VIVNKLDNAK KSNDLSAKQF IEKIYGIEKL KEAKENWGNW YLRNANGKAF | |
| | NDKGKFIKLY TIDNLDEFDN SDFINRNLSD TSYITNALVN HLTFSNSKYK | |
| | YSVVSVNGKQ TSNLRNQIAF VGIKNNKETE REWKRPEGFK SINSNDFLIR | |
| | EEGKNDVKDD VLIKDRSENG HHAEDAYFIT IISQYFRSFK RIERLNVNYR | |
| | KETRELDDLE KNNIKFKEKA SFDNFLLINA LDELNEKLNQ MRFSRMVITK | |
| | KNTQLFNETL YSGKYDKGKN TIKKVEKLNL LDNRTDKIKK IEEFFDEDKL | |
| | KENELTKLHI FNHDKNLYET LKIIWNEVKI EIKNKNLNEK NYFKYFVNKK | |
| | LQEGKISFNE WVPILDNDFK IIRKIRYIKF SSEEKETDEI IFSQSNFLKI | |
| | DQRQNFSFHN TLYWVQIWVY KNQKDQYCFI SIDARNSKFE KDEIKINYEK | |
| | LKTQKEKLQI INEEPILKIN KGDLFENEEK ELFYIVGRDE KPQKLEIKYI | |
| | LGKKIKDQKQ IQKPVKKYFP NWKKVNLTYM GEIFKK | |
| gamma | MTKNYISPIA IDLGAKFTGV ALYQYLEGAD CTQEVAKGLL VDDRGNVTWS | (SEQ |
| proteobacterium | QEGRRGKRHQ VRGYKRRKMA KRLLWLILDS EYGIKREEVT EPLLKFINGL | ID |
| HTCC5015 | LNRRGYTYIS EEVDEESMNV SPLPFSEMMP DYFNSSAPLL EQLAKLLSDK | NO: |
| WP_008284239.1 | NKLVRFRAEG KIPSNKNEFK KLLDTALDGK YKDEKKELSE AWGNILIASE | 100) |
| | NVLKSTVDGH KSRSEYLANI KEDIKSNEEL EKQISSKEID GFYNLVGHLS | |
| | NFQLRLLRKY FNDPNMSGVS YWDEKRLEKY FYQWVQGWHT KGGTDEAEKK | |
| | NIILKTKGAP LLKTLKSLSA DLTIPPYEDQ NNRRPPKCQS VLLSDEKLTM | |
| | HYPKWKEWVG QLVKQNDNAY LNENVTLANA LHRIVERSRS IDPYQLRLLI | |
| | SITDAEKRND LAGYKRLKLS LGSEVDEFLL LVKNIVDETK EAREGLWFET | |
| | ENKLFFKCGK TPPRKEKLKS TLLSAVLGKN LSDDEQSSFI EEFWKSGTPK | |
| | IERRNVRGWC RLASQVQKTY GVYLKEYGLQ QLHKLEAGKK LDDKPLALLY | |
| | KNSGLIASKI GEALNIEPDE VSRFASPHSL AQIFNIIEGD VAGENKTCRA | |
| | CTYENIWRMQ EEKVESLLIN QLLSEIHGER KVPLKSAMCT RLSADSTRPF | |
| | DGQMASIIEH IARKIAQHKI AQINDVPKEF SIDIPIIIES NQFSFTAELE | |
| | EIKRGRGSAK AKKAKELGEK SKAGWVSKTE RIKTSSEGIC PYTGAPLGGS | |
| | GEIDHIIPRS LTGRTKKTVF NSEANLIYCS SKGNHDKGNR VYVIEQLNDK | |
| | YLKKQFSTSD VNLIKKKIKT TIQRFTEGGE KLRSFSELSR EDQKAFRHAL | |
| | FVPELKSEVT SLLAVKNITR VNGTQAWLAK KIASLLAEHL DKQGRDYTLS | |
| | AHQIDPWSVS KQRKMLASAE PIWAKKDPQP AASHVVDAVC TFLEALEQPH | |
| | TASRLKTISS TSFEKTGWRS ALIPDLIKVD ALDRRPKYRR YNIGSTSLFK | |
| | DGIYAERFLP ILIDENGLMA GYDIDNSLKA KGADVVFESL SPFLLFKGEE | |
| | VGAQSLSDWQ ERIDGRYLYM SIDKVKAFDY LQEKVGEKDI AAELLNSIHF | |
| | TQRKTELRAK FSDDSGKKMK TLDAIRKSLK LTVTVNEIGK RKEKCGFSGT | |
| | IGIPAKSAWE NLLDEPLLET YWGTKMPPQE IWEKVYRKHF PRNIPNQAHR | |
| | KVRKDFSLPV VDSVSGGFRV KRKTPNGYNY QLLAIDGYSA VGFKKEGDNV | |
| | DFKSPALVPQ IAESKSVTPI SSELVHLDKN EIVYFDEWRK IDISDSDLKQ | |
| | FVSSLELAPG SQNRFYIRFT VDEDQFERHF KSALRVNGIQ DLDTVNKTFD | |
| | WNREIPSLLI PPRSNLELLE TGQKITFEYI ANGANAEVKK AYSLRRA | |
| Planococcus | MKNYTIGLDI GVASVGWVCI DENYKILNYN NRHAFGVHEF ESAESAAGRR | (SEQ |
| antarcticus | LKRGMRRRYN RRKKRLQLLQ SLFDSYITDS GFFSKTDSQH FWKNNNEFEN | ID |
| DSM 14505 | RSLTEVLSSL RISSRKYPTI YHLRSDLIES NKKMDLRLVY LALHNLVKYR | NO: |
| ANU10858.1 | GHFLQEGNWS EAASAEGMDD QLLELVTRYA ELENLSPLDL SESQWKAAET | 101) |
| | LLLNRNLTKT DQSKELTAMF GKEYEPFCKL VAGLGVSLHQ LFPSSEQALA | |
| | YKETKTKVQL SNENVEEVME LLLEEESALL EAVQPFYQQV VLYELLKGET | |
| | YVAKAKVSAF KQYQKDMASL KNLLDKTFGE KVYRSYFISD KNSQREYQKS | |
| | HKVEVLCKLD QFNKEAKFAE TFYKDLKKLL EDKSKTSIGT TEKDEMLRII | |
| | KAIDSNQFLQ KQKGIQNAAI PHQNSLYEAE KILRNQQAHY PFITTEWIEK | |
| | VKQILAFRIP YYIGPLVKDT TQSPFSWVER KGDAPITPWN FDEQIDKAAS | |
| | AEAFISRMRK TCTYLKGQEV LPKSSLTYER FEVLNELNGI QLRTTGAESD | |
| | FRHRLSYEMK CWIIDNVEKQ YKTVSTKRLL QELKKSPYAD ELYDEHTGEI | |
| | KEVFGTQKEN AFATSLSGYI SMKSILGAVV DDNPAMTEEL IYWIAVFEDR | |
| | EILHLKIQEK YPSITDVQRQ KLALVKLPGW GRFSRLLIDG LPLDEQGQSV | |
| | LDHMEQYSSV FMEVLKNKGF GLEKKIQKMN QHQVDGTKKI RYEDIEELAG | |
| | SPALKRGIWR SVKIVEELVS IFGEPANIVL EVAREDGEKK RTKSRKDQWE | |
| | ELTKTTLKND PDLKSFIGEI KSQGDQRFNE QRFWLYVTQQ GKCLYTGKAL | |
| | DIQNLSMYEV DHILPQNFVK DDSLDNLALV MPEANQRKNQ VGQNKMPLEI | |
| | IEANQQYAMR TLWERLHELK LISSGKLGRL KKPSFDEVDK DKFIARQLVE | |
| | TRQIIKHVRD LLDERFSKSD IHLVKAGIVS KFRRFSEIPK IRDYNNKHHA | |
| | MDALFAAALI QSILGKYGKN FLAFDLSKKD RQKQWRSVKG SNKEFFLFKN | |
| | FGNLRLQSPV TGEEVSGVEY MKHVYFELPW QTTKMTQTGD GMFYKESIFS | |
| | PKVKQAKYVS PKTEKFVHDE VKNHSICLVE FTFMKKEKEV QETKFIDLKV | |
| | IEHHQFLKEP ESQLAKFLAE KETNSPIIHA RIIRTIPKYQ KIWIEHFPYY | |
| | FISTRELHNA RQFEISYELM EKVKQLSERS SVEELKIVFG LLIDQMNDNY | |
| | PIYTKSSIQD RVQKFVDTQL YDFKSFEIGF EELKKAVAAN AQRSDTFGSR | |
| | ISKKPKPEEV AIGYESITGL KYRKPRSVVG TKR | |
| Prevotella | MTQKVLGLDL GTNSIGSAVR NLDLSDDLQW QLEFFSSDIF RSSVNKESNG | (SEQ |
| sp. C561 | REYSLAAQRS AHRRSRGLNE VRRRRLWATL NLLIKHGFCP MSSESLMRWC | ID |
| WP_009013303.1 | TYDKRKGLFR EYPIDDKDEN AWILLDENGD GRPDYSSPYQ LRRELVTRQF | NO: |
| | DFEQPIERYK LGRALYHIAQ HRGFKSSKGE TLSQQETNSK PSSTDEIPDV | 102) |
| | AGAMKASEEK LSKGLSTYMK EHNLLTVGAA FAQLEDEGVR VRNNNDYRAI | |
| | RSQFQHEIET IFKFQQGLSV ESELYERLIS EKKNVGTIFY KRPLRSQRGN | |
| | VGKCTLERSK PRCAIGHPLF EKFRAWTLIN NIKVRMSVDT LDEQLPMKLR | |
| | LDLYNECFLA FVRTEFKFED IRKYLEKRLG IHFSYNDKTI NYKDSTSVAG | |
| | CPITARFRKM LGEEWESFRV EGQKERQAHS KNNISFHRVS YSIEDIWHFC | |
| | YDAEEPEAVL AFAQETLRLE RKKAEELVRI WSAMPQGYAM LSQKAIRNIN | |
| | KILMLGLKYS DAVILAKVPE LVDVSDEELL SIAKDYYLVE AQVNYDKRIN | |
| | SIVIGLIAKY KSVSEEYRFA DHNYEYLLDE SDEKDIIRQI ENSLGARRWS | |
| | LMDANEQTDI LQKVRDRYQD FFRSHERKFV ESPKLGESFE NYLTKKFPMV | |
| | EREQWKKLYH PSQITIYRPV SVGKDRSVLR LGNPDIGAIK NPTVLRVLNT | |
| | LRRRVNQLLD DGVISPDETR VVVETARELN DANRKWALDT YNRIRHDENE | |
| | KIKKILEEFY PKRDGISTDD IDKARYVIDQ REVDYFTGSK TYNKDIKKYK | |
| | FWLEQGGQCM YTGRTINLSN LFDPNAFDIE HTIPESLSFD SSDMNLTLCD | |
| | AHYNRFIKKN HIPTDMPNYD KAITIDGKEY PAITSQLQRW VERVERLNRN | |
| | VEYWKGQARR AQNKDRKDQC MREMHLWKME LEYWKKKLER FTVTEVTDGF | |
| | KNSQLVDTRV ITRHAVLYLK SIFPHVDVQR GDVTAKFRKI LGIQSVDEKK | |
| | DRSLHSHHAI DATTLTIIPV SAKRDRMLEL FAKIEEINKM LSFSGSEDRT | |
| | GLIQELEGLK NKLQMEVKVC RIGHNVSEIG TFINDNIIVN HHIKNQALTP | |
| | VRRRLRKKGY IVGGVDNPRW QTGDALRGEI HKASYYGAIT QFAKDDEGKV | |
| | LMKEGRPQVN PTIKFVIRRE LKYKKSAADS GFASWDDLGK AIVDKELFAL | |
| | MKGQFPAETS FKDACEQGIY MIKKGKNGMP DIKLHHIRHV RCEAPQSGLK | |
| | IKEQTYKSEK EYKRYFYAAV GDLYAMCCYT NGKIREFRIY SLYDVSCHRK | |
| | SDIEDIPEFI TDKKGNRLML DYKLRTGDMI LLYKDNPAEL YDLDNVNLSR | |
| | RLYKINRFES QSNLVLMTHH LSTSKERGRS LGKTVDYQNL PESIRSSVKS | |
| | LNFLIMGENR DFVIKNGKII FNHR | |
| Alicyclobacillus | MAYRLGLDIG ITSVGWAVVA LEKDESGLKP VRIQDLGVRI FDKAEDSKTG | (SEQ |
| hesperidum | ASLALPRREA RSARRRTRRR RHRLWRVKRL LEQHGILSME QIEALYAQRT | ID |
| URH17-3- | SSPDVYALRV AGLDRCLIAE EIARVLIHIA HRRGFQSNRK SEIKDSDAGK | NO: |
| 68 | LLKAVQENEN LMQSKGYRTV AEMLVSEATK TDAEGKLVHG KKHGYVSNVR | 103) |
| WP_006446566.1 | NKAGEYRHTV SRQAIVDEVR KIFAAQRALG NDVMSEELED SYLKILCSQR | |
| | NFDDGPGGDS PYGHGSVSPD GVRQSIYERM VGSCTFETGE KRAPRSSYSF | |
| | ERFQLLTKVV NLRIYRQQED GGRYPCELTQ TERARVIDCA YEQTKITYGK | |
| | LRKLLDMKDT ESFAGLTYGL NRSRNKTEDT VFVEMKFYHE VRKALQRAGV | |
| | FIQDLSIETL DQIGWILSVW KSDDNRRKKL STLGLSDNVI EELLPLNGSK | |
| | FGHLSLKAIR KILPFLEDGY SYDVACELAG YQFQGKTEYV KQRLLPPLGE | |
| | GEVTNPVVRR ALSQAIKVVN AVIRKHGSPE SIHIELAREL SKNLDERRKI | |
| | EKAQKENQKN NEQIKDEIRE ILGSAHVTGR DIVKYKLFKQ QQEFCMYSGE | |
| | KLDVTRLFEP GYAEVDHIIP YGISFDDSYD NKVLVKTEQN RQKGNRTPLE | |
| | YLRDKPEQKA KFIALVESIP LSQKKKNHLL MDKRAIDLEQ EGFRERNLSD | |
| | TRYITRALMN HIQAWLLEDE TASTRSKRVV CVNGAVTAYM RARWGLTKDR | |
| | DAGDKHHAAD AVVVACIGDS LIQRVTKYDK FKRNALADRN RYVQQVSKSE | |
| | GITQYVDKET GEVFTWESFD ERKFLPNEPL EPWPFERDEL LARLSDDPSK | |
| | NIRAIGLLTY SETEQIDPIF VSRMPTRKVT GAAHKETIRS PRIVKVDDNK | |
| | GTEIQVVVSK VALTELKLTK DGEIKDYFRP EDDPRLYNTL RERLVQFGGD | |
| | AKAAFKEPVY KISKDGSVRT PVRKVKIQEK LTLGVPVHGG RGIAENGGMV | |
| | RIDVFAKGGK YYFVPIYVAD VLKRELPNRL ATAHKPYSEW RVVDDSYQFK | |
| | FSLYPNDAVM IKPSREVDIT YKDRKEPVGC RIMYFVSANI ASASISLRTH | |
| | DNSGELEGLG IQGLEVFEKY VVGPLGDTHP VYKERRMPFR VERKMN | |
| Lactobacillus | MTKLNQPYGI GLDIGSNSIG FAVVDANSHL LRLKGETAIG ARLFREGQSA | (SEQ |
| rhamnosus | ADRRGSRTTR RRLSRTRWRL SFLRDFFAPH ITKIDPDFFL RQKYSEISPK | ID |
| GG | DKDRFKYEKR LENDRTDAEF YEDYPSMYHL RLHLMTHTHK ADPREIFLAI | NO: |
| WP_014569977.1 | HHILKSRGHF LTPGAAKDEN TDKVDLEDIF PALTEAYAQV YPDLELTFDL | 104) |
| | AKADDFKAKL LDEQATPSDT QKALVNLLLS SDGEKEIVKK RKQVLTEFAK | |
| | AITGLKTKEN LALGTEVDEA DASNWQFSMG QLDDKWSNIE TSMTDQGTEI | |
| | FEQIQELYRA RLINGIVPAG MSLSQAKVAD YGQHKEDLEL FKTYLKKLND | |
| | HELAKTIRGL YDRYINGDDA KPFLREDFVK ALTKEVTAHP NEVSEQLLNR | |
| | MGQANFMLKQ RTKANGAIPI QLQQRELDQI IANQSKYYDW LAAPNPVEAH | |
| | RWKMPYQLDE LLNFHIPYYV GPLITPKQQA ESGENVFAWM VRKDPSGNIT | |
| | PYNFDEKVDR EASANTFIQR MKTTDTYLIG EDVLPKQSLL YQKYEVLNEL | |
| | NNVRINNECL GTDQKQRLIR EVFERHSSVT IKQVADNLVA HGDFARRPEI | |
| | RGLADEKRFL SSLSTYHQLK EILHEAIDDP TKLLDIENII TWSTVFEDHT | |
| | IFETKLAEIE WLDPKKINEL SGIRYRGWGQ FSRKLLDGLK LGNGHTVIQE | |
| | LMLSNHNLMQ ILADETLKET MTELNQDKLK TDDIEDVIND AYTSPSNKKA | |
| | LRQVLRVVED IKHAANGQDP SWLFIETADG TGTAGKRTQS RQKQIQTVYA | |
| | NAAQELIDSA VRGELEDKIA DKASFTDRLV LYFMQGGRDI YTGAPLNIDQ | |
| | LSHYDIDHIL PQSLIKDDSL DNRVLVNATI NREKNNVFAS TLFAGKMKAT | |
| | WRKWHEAGLI SGRKLRNLML RPDEIDKFAK GFVARQLVET RQIIKLTEQI | |
| | AAAQYPNTKI IAVKAGLSHQ LREELDEPKN RDVNHYHHAF DAFLAARIGT | |
| | YLLKRYPKLA PFFTYGEFAK VDVKKFREFN FIGALTHAKK NIIAKDTGEI | |
| | VWDKERDIRE LDRIYNFKRM LITHEVYFET ADLFKQTIYA AKDSKERGGS | |
| | KQLIPKKQGY PTQVYGGYTQ ESGSYNALVR VAEADTTAYQ VIKISAQNAS | |
| | KIASANLKSR EKGKQLLNEI VVKQLAKRRK NWKPSANSFK IVIPRFGMGT | |
| | LFQNAKYGLF MVNSDTYYRN YQELWLSREN QKLLKKLFSI KYEKTQMNHD | |
| | ALQVYKAIID QVEKFFKLYD INQFRAKLSD AIERFEKLPI NTDGNKIGKT | |
| | ETLRQILIGL QANGTRSNVK NLGIKTDLGL LQVGSGIKLD KDTQIVYQSP | |
| | SGLFKRRIPL ADL | |
| Enterococcus | MYSIGLDLGI SSVGWSVIDE RTGNVIDLGV RLFSAKNSEK NLERRTNRGG | (SEQ |
| faecalis | RRLIRRKTNR LKDAKKILAA VGFYEDKSLK NSCPYQLRVK GLTEPLSRGE | ID |
| TX0012 | IYKVTLHILK KRGISYLDEV DTEAAKESQD YKEQVRKNAQ LLTKYTPGQI | NO: |
| WP_002408901.1 | QLQRLKENNR VKTGINAQGN YQLNVFKVSA YANELATILK TQQAFYPNEL | 105) |
| EFT93846.1 | TDDWIALFVQ PGIAEEAGLI YRKRPYYHGP GNEANNSPYG RWSDFQKTGE | |
| | PATNIFDKLI GKDFQGELRA SGLSLSAQQY NLLNDLTNLK IDGEVPLSSE | |
| | QKEYILTELM TKEFTRFGVN DVVKLLGVKK ERLSGWRLDK KGKPEIHTLK | |
| | GYRNWRKIFA EAGIDLATLP TETIDCLAKV LTLNTEREGI ENTLAFELPE | |
| | LSESVKLLVL DRYKELSQSI STQSWHRFSL KTLHLLIPEL MNATSEQNTL | |
| | LEQFQLKSDV RKRYSEYKKL PTKDVLAEIY NPTVNKTVSQ AFKVIDALLV | |
| | KYGKEQIRYI TIEMPRDDNE EDEKKRIKEL HAKNSQRKND SQSYFMQKSG | |
| | WSQEKFQTTI QKNRRFLAKL LYYYEQDGIC AYTGLPISPE LLVSDSTEID | |
| | HIIPISISLD DSINNKVLVL SKANQVKGQQ TPYDAWMDGS FKKINGKFSN | |
| | WDDYQKWVES RHFSHKKENN LLETRNIFDS EQVEKFLARN LNDTRYASRL | |
| | VLNTLQSFFT NQETKVRVVN GSFTHTLRKK WGADLDKTRE THHHHAVDAT | |
| | LCAVTSFVKV SRYHYAVKEE TGEKVMREID FETGEIVNEM SYWEFKKSKK | |
| | YERKTYQVKW PNFREQLKPV NLHPRIKFSH QVDRKANRKL SDATIYSVRE | |
| | KTEVKTLKSG KQKITTDEYT IGKIKDIYTL DGWEAFKKKQ DKLLMKDLDE | |
| | KTYERLLSIA ETTPDFQEVE EKNGKVKRVK RSPFAVYCEE NDIPAIQKYA | |
| | KKNNGPLIRS LKYYDGKLNK HINITKDSQG RPVEKTKNGR KVTLQSLKPY | |
| | RYDIYQDLET KAYYTVQLYY SDLRFVEGKY GITEKEYMKK VAEQTKGQVV | |
| | RFCFSLQKND GLEIEWKDSQ RYDVRFYNFQ SANSINFKGL EQEMMPAENQ | |
| | FKQKPYNNGA INLNIAKYGK EGKKLRKENT DILGKKHYLF YEKEPKNIIK | |
| Candidatus | MRRLGLDLGT NSIGWCLLDL GDDGEPVSIF RTGARIFSDG RDPKSLGSLK | (SEQ |
| Puniceispirillum | ATRREARLTR RRRDRFIQRQ KNLINALVKY GLMPADEIQR QALAYKDPYP | ID |
| marinum | IRKKALDEAI DPYEMGRAIF HINQRRGFKS NRKSADNEAG VVKQSIADLE | NO: |
| IMCC1322 | MKLGEAGART IGEFLADRQA TNDTVRARRL SGTNALYEFY PDRYMLEQEF | 106) |
| WP_013047413.1 | DTLWAKQAAF NPSLYIEAAR ERLKEIVFFQ RKLKPQEVGR CIFLSDEDRI | |
| | SKALPSFQRF RIYQELSNLA WIDHDGVAHR ITASLALRDH LFDELEHKKK | |
| | LTFKAMRAIL RKQGVVDYPV GENLESDNRD HLIGNLTSCI MRDAKKMIGS | |
| | AWDRLDEEEQ DSFILMLQDD QKGDDEVRSI LTQQYGLSDD VAEDCLDVRL | |
| | PDGHGSLSKK AIDRILPVLR DQGLIYYDAV KEAGLGEANL YDPYAALSDK | |
| | LDYYGKALAG HVMGASGKFE DSDEKRYGTI SNPTVHIALN QVRAVVNELI | |
| | RLHGKPDEVV IEIGRDLPMG ADGKRELERF QKEGRAKNER ARDELKKLGH | |
| | IDSRESRQKF QLWEQLAKEP VDRCCPFTGK MMSISDLFSD KVEIEHLLPF | |
| | SLTLDDSMAN KTVCFRQANR DKGNRAPFDA FGNSPAGYDW QEILGRSQNL | |
| | PYAKRWRFLP DAMKRFEADG GFLERQLNDT RYISRYTTEY ISTIIPKNKI | |
| | WVVTGRLTSL LRGFWGLNSI LRGHNTDDGT PAKKSRDDHR HHAIDAIVVG | |
| | MTSRGLLQKV SKAARRSEDL DLTRLFEGRI DPWDGFRDEV KKHIDAIIVS | |
| | HRPRKKSQGA LHNDTAYGIV EHAENGASTV VHRVPITSLG KQSDIEKVRD | |
| | PLIKSALLNE TAGLSGKSFE NAVQKWCADN SIKSLRIVET VSIIPITDKE | |
| | GVAYKGYKGD GNAYMDIYQD PTSSKWKGEI VSREDANQKG FIPSWQSQFP | |
| | TARLIMRLRI NDLLKLQDGE IEEIYRVQRL SGSKILMAPH TEANVDARDR | |
| | DKNDTFKLTS KSPGKLQSAS ARKVHISPTG LIREG | |
| Oenococcus | MARDYSVGLD IGTSSVGWAA IDNKYHLIRA KSKNLIGVRL FDSAVTAEKR | (SEQ |
| kitaharae | RGYRTTRRRL SRRHWRLRLL NDIFAGPLTD FGDENFLARL KYSWVHPQDQ | ID |
| DSM 17330 | SNQAHFAAGL LFDSKEQDKD FYRKYPTIYH LRLALMNDDQ KHDLREVYLA | NO: |
| EHN59352.1 | IHHLVKYRGH FLIEGDVKAD SAFDVHTFAD AIQRYAESNN SDENLLGKID | 107) |
| | EKKLSAALTD KHGSKSQRAE TAETAFDILD LQSKKQIQAI LKSVVGNQAN | |
| | LMAIFGLDSS AISKDEQKNY KFSFDDADID EKIADSEALL SDTEFEFLCD | |
| | LKAAFDGLTL KMLLGDDKTV SAAMVRRFNE HQKDWEYIKS HIRNAKNAGN | |
| | GLYEKSKKFD GINAAYLALQ SDNEDDRKKA KKIFQDEISS ADIPDDVKAD | |
| | FLKKIDDDQF LPIQRTKNNG TIPHQLHRNE LEQIIEKQGI YYPFLKDTYQ | |
| | ENSHELNKIT ALINFRVPYY VGPLVEEEQK IADDGKNIPD PTNHWMVRKS | |
| | NDTITPWNLS QVVDLDKSGR RFIERLTGTD TYLIGEPTLP KNSLLYQKED | |
| | VLQELNNIRV SGRRLDIRAK QDAFEHLFKV QKTVSATNLK DFLVQAGYIS | |
| | EDTQIEGLAD VNGKNENNAL TTYNYLVSVL GREFVENPSN EELLEEITEL | |
| | QTVFEDKKVL RRQLDQLDGL SDHNREKLSR KHYTGWGRIS KKLLTTKIVQ | |
| | NADKIDNQTF DVPRMNQSII DTLYNTKMNL MEIINNAEDD FGVRAWIDKQ | |
| | NTTDGDEQDV YSLIDELAGP KEIKRGIVQS FRILDDITKA VGYAPKRVYL | |
| | EFARKTQESH LTNSRKNQLS TLLKNAGLSE LVTQVSQYDA AALQNDRLYL | |
| | YFLQQGKDMY SGEKLNLDNL SNYDIDHIIP QAYTKDNSLD NRVLVSNITN | |
| | RRKSDSSNYL PALIDKMRPF WSVLSKQGLL SKHKFANLTR TRDEDDMEKE | |
| | RFIARSLVET RQIIKNVASL IDSHFGGETK AVAIRSSLTA DMRRYVDIPK | |
| | NRDINDYHHA FDALLFSTVG QYTENSGLMK KGQLSDSAGN QYNRYIKEWI | |
| | HAARLNAQSQ RVNPFGFVVG SMRNAAPGKL NPETGEITPE ENADWSIADL | |
| | DYLHKVMNER KITVTRRLKD QKGQLYDESR YPSVLHDAKS KASINEDKHK | |
| | PVDLYGGFSS AKPAYAALIK FKNKFRLVNV LRQWTYSDKN SEDYILEQIR | |
| | GKYPKAEMVL SHIPYGQLVK KDGALVTISS ATELHNFEQL WLPLADYKLI | |
| | NTLLKTKEDN LVDILHNRLD LPEMTIESAF YKAFDSILSF AFNRYALHQN | |
| | ALVKLQAHRD DENALNYEDK QQTLERILDA LHASPASSDL KKINLSSGFG | |
| | RLFSPSHFTL ADTDEFIFQS VTGLFSTQKT VAQLYQETK | |
| Helicobacter | MIRTLGIDIG IASIGWAVIE GEYTDKGLEN KEIVASGVRV FTKAENPKNK | (SEQ |
| mustelae | ESLALPRTLA RSARRRNARK KGRIQQVKHY LSKALGLDLE CFVQGEKLAT | ID |
| 12198 | LFQTSKDFLS PWELRERALY RVLDKEELAR VILHIAKRRG YDDITYGVED | NO: |
| WP_013022389.1 | NDSGKIKKAI AENSKRIKEE QCKTIGEMMY KLYFQKSLNV RNKKESYNRC | 108) |
| | VGRSELREEL KTIFQIQQEL KSPWVNEELI YKLLGNPDAQ SKQEREGLIF | |
| | YQRPLKGFGD KIGKCSHIKK GENSPYRACK HAPSAEEFVA LIKSINFLKN | |
| | LTNRHGLCFS QEDMCVYLGK ILQEAQKNEK GLTYSKLKLL LDLPSDFEFL | |
| | GLDYSGKNPE KAVFLSLPST FKLNKITQDR KTQDKIANIL GANKDWEAIL | |
| | KELESLQLSK EQIQTIKDAK LNFSKHINLS LEALYHLLPL MREGKRYDEG | |
| | VEILQERGIF SKPQPKNRQL LPPLSELAKE ESYFDIPNPV LRRALSEFRK | |
| | VVNALLEKYG GFHYFHIELT RDVCKAKSAR MQLEKINKKN KSENDAASQL | |
| | LEVLGLPNTY NNRLKCKLWK QQEEYCLYSG EKITIDHLKD QRALQIDHAF | |
| | PLSRSLDDSQ SNKVLCLTSS NQEKSNKTPY EWLGSDEKKW DMYVGRVYSS | |
| | NFSPSKKRKL TQKNFKERNE EDFLARNLVD TGYIGRVTKE YIKHSLSFLP | |
| | LPDGKKEHIR IISGSMTSTM RSFWGVQEKN RDHHLHHAQD AIIIACIEPS | |
| | MIQKYTTYLK DKETHRLKSH QKAQILREGD HKLSLRWPMS NFKDKIQESI | |
| | QNIIPSHHVS HKVTGELHQE TVRTKEFYYQ AFGGEEGVKK ALKFGKIREI | |
| | NQGIVDNGAM VRVDIFKSKD KGKFYAVPIY TYDFAIGKLP NKAIVQGKKN | |
| | GIIKDWLEMD ENYEFCFSLF KNDCIKIQTK EMQEAVLAIY KSTNSAKATI | |
| | ELEHLSKYAL KNEDEEKMFT DTDKEKNKTM TRESCGIQGL KVFQKVKLSV | |
| | LGEVLEHKPR NRQNIALKTT PKHV | |
| Bradyrhizobium | MKRTSLRAYR LGVDLGANSL GWFVVWLDDH GQPEGLGPGG VRIFPDGRNP | (SEQ |
| sp. | QSKQSNAAGR RLARSARRRR DRYLQRRGKL MGLLVKHGLM PADEPARKRL | ID |
| BTAi1 | ECLDPYGLRA KALDEVLPLH HVGRALFHLN QRRGLFANRA IEQGDKDASA | NO: |
| WP_012044026.1 | IKAAAGRLQT SMQACGARTL GEFLNRRHQL RATVRARSPV GGDVQARYEF | 109) |
| | YPTRAMVDAE FEAIWAAQAP HHPTMTAEAH DTIREAIFSQ RAMKRPSIGK | |
| | CSLDPATSQD DVDGFRCAWS HPLAQRFRIW QDVRNLAVVE TGPTSSRLGK | |
| | EDQDKVARAL LQTDQLSFDE IRGLLGLPSD ARFNLESDRR DHLKGDATGA | |
| | ILSARRHFGP AWHDRSLDRQ IDIVALLESA LDEAAIIASL GTTHSLDEAA | |
| | AQRALSALLP DGYCRLGLRA IKRVLPLMEA GRTYAEAASA AGYDHALLPG | |
| | GKLSPTGYLP YYGQWLQNDV VGSDDERDTN ERRWGRLPNP TVHIGIGQLR | |
| | RVVNELIRWH GPPAEITVEL TRDLKLSPRR LAELEREQAE NQRKNDKRIS | |
| | LLRKLGLPAS THNLLKLRLW DEQGDVASEC PYTGEAIGLE RLVSDDVDID | |
| | HLIPFSISWD DSAANKVVCM RYANREKGNR TPFEAFGHRQ GRPYDWADIA | |
| | ERAARLPRGK RWRFGPGARA QFEELGDFQA RLLNETSWLA RVAKQYLAAV | |
| | THPHRIHVLP GRLTALLRAT WELNDLLPGS DDRAAKSRKD HRHHAIDALV | |
| | AALTDQALLR RMANAHDDTR RKIEVLLPWP TFRIDLETRL KAMLVSHKPD | |
| | HGLQARLHED TAYGTVEHPE TEDGANLVYR KTFVDISEKE IDRIRDRRLR | |
| | DLVRAHVAGE RQQGKTLKAA VLSFAQRRDI AGHPNGIRHV RLTKSIKPDY | |
| | LVPIRDKAGR IYKSYNAGEN AFVDILQAES GRWIARATTV FQANQANESH | |
| | DAPAAQPIMR VFKGDMLRID HAGAEKFVKI VRLSPSNNLL YLVEHHQAGV | |
| | FQTRHDDPED SFRWLFASED KLREWNAELV RIDTLGQPWR RKRGLETGSE | |
| | DATRIGWTRP KKWP | |
| Acidaminococcus | MGKMYYLGLD IGTNSVGYAV TDPSYHLLKF KGEPMWGAHV FAAGNQSAER | (SEQ |
| sp. | RSFRTSRRRL DRRQQRVKLV QEIFAPVISP IDPRFFIRLH ESALWRDDVA | ID |
| D21 | ETDKHIFEND PTYTDKEYYS DYPTIHHLIV DLMESSEKHD PRLVYLAVAW | NO: |
| WP_009016219.1 | LVAHRGHFLN EVDKDNIGDV LSFDAFYPEF LAFLSDNGVS PWVCESKALQ | 110) |
| | ATLLSRNSVN DKYKALKSLI FGSQKPEDNF DANISEDGLI QLLAGKKVKV | |
| | NKLFPQESND ASFTLNDKED AIEEILGTLT PDECEWIAHI RRLFDWAIMK | |
| | HALKDGRTIS ESKVKLYEQH HHDLTQLKYF VKTYLAKEYD DIFRNVDSET | |
| | TKNYVAYSYH VKEVKGTLPK NKATQEEFCK YVLGKVKNIE CSEADKVDFD | |
| | EMIQRLTDNS FMPKQVSGEN RVIPYQLYYY ELKTILNKAA SYLPELTQCG | |
| | KDAISNQDKL LSIMTFRIPY FVGPLRKDNS EHAWLERKAG KIYPWNENDK | |
| | VDLDKSEEAF IRRMINTCTY YPGEDVLPLD SLIYEKFMIL NEINNIRIDG | |
| | YPISVDVKQQ VFGLFEKKRR VTVKDIQNLL LSLGALDKHG KLTGIDTTIH | |
| | SNYNTYHHFK SLMERGVLTR DDVERIVERM TYSDDTKRVR LWLNNNYGTL | |
| | TADDVKHISR LRKHDFGRLS KMFLTGLKGV HKETGERASI LDEMWNTNDN | |
| | LMQLLSECYT FSDEITKLQE AYYAKAQLSL NDFLDSMYIS NAVKRPIYRT | |
| | LAVVNDIRKA CGTAPKRIFI EMARDGESKK KRSVTRREQI KNLYRSIRKD | |
| | FQQEVDFLEK ILENKSDGQL QSDALYLYFA QLGRDMYTGD PIKLEHIKDQ | |
| | SFYNIDHIYP QSMVKDDSLD NKVLVQSEIN GEKSSRYPLD AAIRNKMKPL | |
| | WDAYYNHGLI SLKKYQRLTR STPFTDDEKW DFINRQLVET RQSTKALAIL | |
| | LKRKFPDTEI VYSKAGLSSD FRHEFGLVKS RNINDLHHAK DAFLAIVTGN | |
| | VYHERFNRRW FMVNQPYSVK TKTLFTHSIK NGNFVAWNGE EDLGRIVKML | |
| | KQNKNTIHFT RFSFDRKEGL FDIQPLKAST GLVPRKAGLD VVKYGGYDKS | |
| | TAAYYLLVRF TLEDKKTQHK LMMIPVEGLY KARIDHDKEF LTDYAQTTIS | |
| | EILQKDKQKV INIMFPMGTR HIKLNSMISI DGFYLSIGGK SSKGKSVLCH | |
| | AMVPLIVPHK IECYIKAMES FARKFKENNK LRIVEKEDKI TVEDNLNLYE | |
| | LFLQKLQHNP YNKFFSTQFD VLINGRSTFT KLSPEEQVQT LLNILSIFKT | |
| | CRSSGCDLKS INGSAQAARI MISADLTGLS KKYSDIRLVE QSASGLFVSK | |
| | SQNLLEYL | |
| Methylosinus | MRVLGLDAGI ASLGWALIEI EESNRGELSQ GTIIGAGTWM FDAPEEKTQA | (SEQ |
| trichosporium | GAKLKSEQRR TFRGQRRVVR RRRQRMNEVR RILHSHGLLP SSDRDALKQP | ID |
| OB3b | GLDPWRIRAE ALDRLLGPVE LAVALGHIAR HRGFKSNSKG AKTNDPADDT | NO: |
| WP_003611034.1 | SKMKRAVNET REKLARFGSA AKMLVEDESF VLRQTPTKNG ASEIVRRERN | 111) |
| | REGDYSRSLL RDDLAAEMRA LFTAQARFQS AIATADLQTA FTKAAFFQRP | |
| | LQDSEKLVGP CPFEVDEKRA PKRGYSFELF RFLSRLNHVT LRDGKQERTL | |
| | TRDELALAAA DFGAAAKVSF TALRKKLKLP ETTVFVGVKA DEESKLDVVA | |
| | RSGKAAEGTA RLRSVIVDAL GELAWGALLC SPEKLDKIAE VISERSDIGR | |
| | ISEGLAQAGC NAPLVDALTA AASDGRFDPF TGAGHISSKA ARNILSGLRQ | |
| | GMTYDKACCA ADYDHTASRE RGAFDVGGHG REALKRILQE ERISRELVGS | |
| | PTARKALIES IKQVKAIVER YGVPDRIHVE LARDVGKSIE EREEITRGIE | |
| | KRNRQKDKLR GLFEKEVGRP PQDGARGKEE LLRFELWSEQ MGRCLYTDDY | |
| | ISPSQLVATD DAVQVDHILP WSRFADDSYA NKTLCMAKAN QDKKGRTPYE | |
| | WFKAEKTDTE WDAFIVRVEA LADMKGFKKR NYKLRNAEEA AAKFRNRNLN | |
| | DTRWACRLLA EALKQLYPKG EKDKDGKERR RVFSRPGALT DRLRRAWGLQ | |
| | WMKKSTKGDR IPDDRHHALD AIVIAATTES LLQRATREVQ EIEDKGLHYD | |
| | LVKNVTPPWP GFREQAVEAV EKVFVARAER RRARGKAHDA TIRHIAVREG | |
| | EQRVYERRKV AELKLADLDR VKDAERNARL IEKLRNWIEA GSPKDDPPLS | |
| | PKGDPIFKVR LVTKSKVNIA LDTGNPKRPG TVDRGEMARV DVFRKASKKG | |
| | KYEYYLVPIY PHDIATMKTP PIRAVQAYKP EDEWPEMDSS YEFCWSLVPM | |
| | TYLQVISSKG EIFEGYYRGM NRSVGAIQLS AHSNSSDVVQ GIGARTLTEF | |
| | KKFNVDRFGR KHEVERELRT WRGETWRGKA YI | |
| Actinomyces | MDNKNYRIGI DVGLNSIGFC AVEVDQHDTP LGFLNLSVYR HDAGIDPNGK | (SEQ |
| coleocanis | KTNTTRLAMS GVARRTRRLF RKRKRRLAAL DRFIEAQGWT LPDHADYKDP | ID |
| DSM 15436 | YTPWLVRAEL AQTPIRDEND LHEKLAIAVR HIARHRGWRS PWVPVRSLHV | NO: |
| WP_006546479.1 | EQPPSDQYLA LKERVEAKTL LQMPEGATPA EMVVALDLSV DVNLRPKNRE | 112) |
| | KTDTRPENKK PGFLGGKLMQ SDNANELRKI AKIQGLDDAL LRELIELVFA | |
| | ADSPKGASGE LVGYDVLPGQ HGKRRAEKAH PAFQRYRIAS IVSNLRIRHL | |
| | GSGADERLDV ETQKRVFEYL LNAKPTADIT WSDVAEEIGV ERNLLMGTAT | |
| | QTADGERASA KPPVDVINVA FATCKIKPLK EWWLNADYEA RCVMVSALSH | |
| | AEKLTEGTAA EVEVAEFLQN LSDEDNEKLD SFSLPIGRAA YSVDSLERLT | |
| | KRMIENGEDL FEARVNEFGV SEDWRPPAEP IGARVGNPAV DRVLKAVNRY | |
| | LMAAEAEWGA PLSVNIEHVR EGFISKRQAV EIDRENQKRY QRNQAVRSQI | |
| | ADHINATSGV RGSDVTRYLA IQRQNGECLY CGTAITFVNS EMDHIVPRAG | |
| | LGSTNTRDNL VATCERCNKS KSNKPFAVWA AECGIPGVSV AEALKRVDFW | |
| | IADGFASSKE HRELQKGVKD RLKRKVSDPE IDNRSMESVA WMARELAHRV | |
| | QYYFDEKHTG TKVRVFRGSL TSAARKASGF ESRVNFIGGN GKTRLDRRHH | |
| | AMDAATVAML RNSVAKTLVL RGNIRASERA IGAAETWKSF RGENVADRQI | |
| | FESWSENMRV LVEKENLALY NDEVSIFSSL RLQLGNGKAH DDTITKLQMH | |
| | KVGDAWSLTE IDRASTPALW CALTRQPDET WKDGLPANED RTIIVNGTHY | |
| | GPLDKVGIFG KAAASLLVRG GSVDIGSAIH HARIYRIAGK KPTYGMVRVF | |
| | APDLLRYRNE DLFNVELPPQ SVSMRYAEPK VREAIREGKA EYLGWLVVGD | |
| | ELLLDLSSET SGQIAELQQD FPGTTHWTVA GFFSPSRLRL RPVYLAQEGL | |
| | GEDVSEGSKS IIAGQGWRPA VNKVFGSAMP EVIRRDGLGR KRRFSYSGLP | |
| | VSWQG | |
| Caenispirillum | MPVLSPLSPN AAQGRRRWSL ALDIGEGSIG WAVAEVDAEG RVLQLTGTGV | (SEQ |
| salinarum | TLFPSAWSNE NGTYVAHGAA DRAVRGQQQR HDSRRRRLAG LARLCAPVLE | ID |
| AK4 | RSPEDLKDLT RTPPKADPRA IFFLRADAAR RPLDGPELFR VLHHMAAHRG | NO: |
| WP_009541330.1 | IRLAELQEVD PPPESDADDA APAATEDEDG TRRAAADERA FRRLMAEHMH | 113) |
| | RHGTQPTCGE IMAGRLRETP AGAQPVTRAR DGLRVGGGVA VPTRALIEQE | |
| | FDAIRAIQAP RHPDLPWDSL RRLVLDQAPI AVPPATPCLF LEELRRRGET | |
| | FQGRTITREA IDRGLTVDPL IQALRIRETV GNLRLHERIT EPDGRQRYVP | |
| | RAMPELGLSH GELTAPERDT LVRALMHDPD GLAAKDGRIP YTRLRKLIGY | |
| | DNSPVCFAQE RDTSGGGITV NPTDPLMARW IDGWVDLPLK ARSLYVRDVV | |
| | ARGADSAALA RLLAEGAHGV PPVAAAAVPA ATAAILESDI MQPGRYSVCP | |
| | WAAEAILDAW ANAPTEGFYD VTRGLFGFAP GEIVLEDLRR ARGALLAHLP | |
| | RTMAAARTPN RAAQQRGPLP AYESVIPSQL ITSLRRAHKG RAADWSAADP | |
| | EERNPFLRTW TGNAATDHIL NQVRKTANEV ITKYGNRRGW DPLPSRITVE | |
| | LAREAKHGVI RRNEIAKENR ENEGRRKKES AALDTFCQDN TVSWQAGGLP | |
| | KERAALRLRL AQRQEFFCPY CAERPKLRAT DLFSPAETEI DHVIERRMGG | |
| | DGPDNLVLAH KDCNNAKGKK TPHEHAGDLL DSPALAALWQ GWRKENADRL | |
| | KGKGHKARTP REDKDFMDRV GWRFEEDARA KAEENQERRG RRMLHDTARA | |
| | TRLARLYLAA AVMPEDPAEI GAPPVETPPS PEDPTGYTAI YRTISRVQPV | |
| | NGSVTHMLRQ RLLQRDKNRD YQTHHAEDAC LLLLAGPAVV QAFNTEAAQH | |
| | GADAPDDRPV DLMPTSDAYH QQRRARALGR VPLATVDAAL ADIVMPESDR | |
| | QDPETGRVHW RLTRAGRGLK RRIDDLTRNC VILSRPRRPS ETGTPGALHN | |
| | ATHYGRREIT VDGRTDTVVT QRMNARDLVA LLDNAKIVPA ARLDAAAPGD | |
| | TILKEICTEI ADRHDRVVDP EGTHARRWIS ARLAALVPAH AEAVARDIAE | |
| | LADLDALADA DRTPEQEARR SALRQSPYLG RAISAKKADG RARAREQEIL | |
| | TRALLDPHWG PRGLRHLIMR EARAPSLVRI RANKTDAFGR PVPDAAVWVK | |
| | TDGNAVSQLW RLTSVVTDDG RRIPLPKPIE KRIEISNLEY ARLNGLDEGA | |
| | GVTGNNAPPR PLRQDIDRLT PLWRDHGTAP GGYLGTAVGE LEDKARSALR | |
| | GKAMRQTLTD AGITAEAGWR LDSEGAVCDL EVAKGDTVKK DGKTYKVGVI | |
| | TQGIFGMPVD AAGSAPRTPE DCEKFEEQYG IKPWKAKGIP LA | |
| Coriobacterium | MKLRGIEDDY SIGLDMGTSS VGWAVTDERG TLAHFKRKPT WGSRLFREAQ | (SEQ |
| glomerans | TAAVARMPRG QRRRYVRRRW RLDLLQKLFE QQMEQADPDF FIRLRQSRLL | ID |
| PW2 | RDDRAEEHAD YRWPLENDCK FTERDYYQRF PTIYHVRSWL METDEQADIR | NO: |
| WP_013709575.1 | LIYLALHNIV KHRGNFLREG QSLSAKSARP DEALNHLRET LRVWSSERGF | 114) |
| | ECSIADNGSI LAMLTHPDLS PSDRRKKIAP LFDVKSDDAA ADKKLGIALA | |
| | GAVIGLKTEF KNIFGDFPCE DSSIYLSNDE AVDAVRSACP DDCAELFDRL | |
| | CEVYSAYVLQ GLLSYAPGQT ISANMVEKYR RYGEDLALLK KLVKIYAPDQ | |
| | YRMFFSGATY PGTGIYDAAQ ARGYTKYNLG PKKSEYKPSE SMQYDDERKA | |
| | VEKLFAKTDA RADERYRMMM DREDKQQFLR RLKTSDNGSI YHQLHLEELK | |
| | AIVENQGRFY PFLKRDADKL VSLVSFRIPY YVGPLSTRNA RTDQHGENRE | |
| | AWSERKPGMQ DEPIFPWNWE SIIDRSKSAE KFILRMTGMC TYLQQEPVLP | |
| | KSSLLYEEFC VLNELNGAHW SIDGDDEHRF DAADREGIIE ELFRRKRTVS | |
| | YGDVAGWMER ERNQIGAHVC GGQGEKGFES KLGSYIFFCK DVFKVERLEQ | |
| | SDYPMIERII LWNTLFEDRK ILSQRLKEEY GSRLSAEQIK TICKKRFTGW | |
| | GRLSEKFLIG ITVQVDEDSV SIMDVLREGC PVSGKRGRAM VMMEILRDEE | |
| | LGFQKKVDDF NRAFFAENAQ ALGVNELPGS PAVRRSLNQS IRIVDEIASI | |
| | AGKAPANIFI EVTRDEDPKK KGRRTKRRYN DLKDALEAFK KEDPELWREL | |
| | CETAPNDMDE RLSLYFMQRG KCLYSGRAID IHQLSNAGIY EVDHIIPRTY | |
| | VKDDSLENKA LVYREENQRK TDMLLIDPEI RRRMSGYWRM LHEAKLIGDK | |
| | KERNLLRSRI DDKALKGFIA RQLVETGQMV KLVRSLLEAR YPETNIISVK | |
| | ASISHDLRTA AELVKCREAN DFHHAHDAFL ACRVGLFIQK RHPCVYENPI | |
| | GLSQVVRNYV RQQADIFKRC RTIPGSSGFI VNSFMTSGED KETGEIFKDD | |
| | WDAEAEVEGI RRSLNFRQCF ISRMPFEDHG VFWDATIYSP RAKKTAALPL | |
| | KQGLNPSRYG SFSREQFAYF FIYKARNPRK EQTLFEFAQV PVRLSAQIRQ | |
| | DENALERYAR ELAKDQGLEF IRIERSKILK NQLIEIDGDR LCITGKEEVR | |
| | NACELAFAQD EMRVIRMLVS EKPVSRECVI SLENRILLHG DQASRRLSKQ | |
| | LKLALLSEAF SEASDNVQRN VVLGLIAIFN GSTNMVNLSD IGGSKFAGNV | |
| | RIKYKKELAS PKVNVHLIDQ SVTGMFERRT KIGL | |

In some embodiments, prime editors utilized herein comprise CRISPR-Cas system enzymes other than type II enzymes. In certain embodiments, prime editors comprise type V or type VI CRISPR-Cas system enzymes. It will be appreciated that certain CRISPR enzymes exhibit promiscuous ssDNA cleavage activity and appropriate precautions should be considered. In certain embodiments, prime editors comprise a nickase or a dead CRISPR with nuclease function comprised in a different component.

In various embodiments, the nucleic acid programmable DNA binding proteins utilized herein include, without limitation, Cas9 (e.g., dCas9 and nCas9), Cas12a (Cpf1), Cas12b1 (C2c1), Cas12b2, Cas12c (C2c3), Cas12d (CasY), Cas12e (CasX), C2c4, C2c5, C2c8, C2c9, C2c10, Cas13a (C2c2), Cas13b (C2c6), Cas13c (C2c7), Cas13d, and Argonaute. Cas-equivalents further include those described in Makarova et al., “C2c2 is a single-component programmable RNA-guided RNA-targeting CRISPR effector,” Science 2016; 353(6299) and Makarova et al., “Classification and Nomenclature of CRISPR-Cas Systems: Where from Here?,” The CRISPR Journal, Vol. 1. No. 5, 2018, the contents of which are incorporated herein by reference. One example of a nucleic acid programmable DNA-binding protein that has different PAM specificity than Cas9 is Clustered Regularly Interspaced Short Palindromic Repeats from Prevotella and Francisella 1 (i.e, Cas12a (Cpf1)). Similar to Cas9, Cas12a (Cpf1) is also a Class 2 CRISPR effector, but it is a member of type V subgroup of enzymes, rather than the type II subgroup. It has been shown that Cas12a (Cpf1) mediates robust DNA interference with features distinct from Cas9. Cas12a (Cpf1) is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference.

### 6.3. Type V CRISPR Proteins

In some embodiments, prime editors used herein comprise the type V CRISPR family includes Francisella novicida U112 Cpf1 (FnCpf1) also known as FnCas12a. FnCpf1 adopts a bilobed architecture with the two lobes connected by the wedge (WED) domain. The N-terminal REC lobe consists of two a-helical domains (REC1 and REC2) that have been shown to coordinate the crRNA-target DNA heteroduplex. The C-terminal NUC lobe consists of the C-terminal RuvC and Nuc domains involved in target cleavage, the arginine-rich bridge helix (BH), and the PAM-interacting (PI) domain. The repeat-derived segment of the crRNA forms a pseudoknot stabilized by intra-molecular base-pairing and hydrogen-bonding interactions. The pseudoknot is coordinated by residues from the WED, RuvC, and REC2 domains, as well as by two hydrated magnesium cations. Notably, nucleotides 1-5 of the crRNA are ordered in the central cavity of FnCas12a and adopt an A-form-like helical conformation. Conformational ordering of the seed sequence is facilitated by multiple interactions between the ribose and phosphate moieties of the crRNA backbone and FnCpf1 residues in the WED and REC1 domains. These include residues Thr16, Lys595, His804, and His881 from the WED domain and residues Tyr47, Lys51, Phe182, and Arg186 from the REC1 domain. The structure of the FnCas12a-crRNA complex further reveals that the bases of the seed sequence are solvent exposed and poised for hybridization with target DNA. Structural aspects of FnCpf1 are described by Swarts et al., Structural Basis for Guide RNA Processing and Seed-Dependent DNA Targeting by CRISPR-Cas12a, Molecular Cell 66, 221-233, Apr. 20, 2017.

Pre-crRNA processing: Essential residues for crRNA processing include His843, Lys852, and Lys869. Structural observations are consistent with an acid-base catalytic mechanism in which Lys869 acts as the general base catalyst to deprotonate the attacking 2′-hydroxyl group of U(−19), while His843 acts as a general acid to protonate the 5′-oxygen leaving group of A(−18). In turn, the side chain of Lys852 is involved in charge stabilization of the transition state. Collectively, these interactions facilitate the intra-molecular attack of the 20-hydroxyl group of U(−19) on the scissile phosphate and promote the formation of the 2′,3′-cyclic phosphate product.

R-loop formation: The crRNA-target DNA strand heteroduplex is enclosed in the central cavity formed by the REC and NUC lobes and interacts extensively with the REC1 and REC2 domains. The PAM-containing DNA duplex comprises target strand nucleotides dT0-dT8 and non-target strand nucleotides dA(8)*-dA0* and is contacted by the PI, WED, and REC1 domains. The 5′-TTN-3′ PAM is recognized in FnCas12a by a mechanism combining the shape-specific recognition of a narrowed minor groove, with base-specific recognition of the PAM bases by two invariant residues, Lys671 and Lys613. Directly downstream of the PAM, the duplex of the target DNA is disrupted by the side chain of residue Lys667, which is inserted between the DNA strands and forms a cation-n stacking interaction with the dA0-dT0* base pair. The phosphate group linking target strand residues dT(−1) and dT0 is coordinated by hydrogen-bonding interactions with the side chain of Lys823 and the backbone amide of Gly826. Target strand residue dT(−1) bends away from residue TO, allowing the target strand to interact with the seed sequence of the crRNA. The non-target strand nucleotides dT1*-dT5* interact with the Arg692-Ser702 loop in FnCas12a through hydrogen-bonding and ionic interactions between backbone phosphate groups and side chains of Arg692, Asn700, Ser702, and Gln704, as well as main-chain amide groups of Lys699, Asn700, and Ser702. Alanine substitution of Q704 or replacement of residues Thr698-Ser702 in FnCas12a with the sequence Ala-Gly3 (SEQ ID NO: 115) substantially reduced DNA cleavage activity, suggesting that these residues contribute to R-loop formation by stabilizing the displaced conformation of the nontarget DNA strand.

In the FnCas12a R-loop complex, the crRNA-target strand heteroduplex is terminated by a stacking interaction with a conserved aromatic residue (Tyr410). This prevents base pairing between the crRNA and the target strand beyond nucleotides U20 and dA(−20), respectively. Beyond this point, the target DNA strand nucleotides re-engage the non-target DNA strand, forming a PAM-distal DNA duplex comprising nucleotides dC(−21)-dA(−27) and dG21*-dT27*, respectively. The duplex is confined between the REC2 and Nuc domains at the end of the central channel formed by the REC and NUC lobes.

Target DNA cleavage: FnCpf1 can independently accommodate both the target and non-target DNA strands in the catalytic pocket of the RuvC domain. The RuvC active site contains three catalytic residues (D917, E1006, and D1255). Structural observations suggest that both the target and non-target DNA strands are cleaved by the same catalytic mechanism in a single active site in Cpf1/Cas12a enzymes.

Another type V CRISPR is AsCpf1 from Acidaminococcus sp BV3L6 (Yamano et al., Crystal structure of Cpf1 in complex with guide RNA and target DNA, Cell 165, 949-962, May 5, 2016)

In certain embodiments, the nuclease comprises a Cas12f effector. Small CRISPR-associated effector proteins belonging to the type V-F subtype have been identified through the mining of sequence databases and members classified into Cas12f1 (Cas14a and type V-U3), Cas12f2 (Cas14b) and Cas12f3 (Cas14c, type V-U2 and U4). (See, e.g., Karvelis et al., PAM recognition by miniature CRISPR-Cas12f nucleases triggers programmable double-stranded DNA target cleavage. Nucleic Acids Research, 21 May 2020, 48(9), 5016-23 doi.org/10.1093/nar/gkaa208). Xu et al. described development of a 529 amino acid Cas12f-based system for mammalian genome engineering through multiple rounds of iterative protein engineering and screening. (Xu, X. et al., Engineered Miniature CRISPR-Cas System for Mammalian Genome Regulation and Editing. Molecular Cell, Oct. 21, 2021, 81(20): 4333-45, doi.org/10.1016/j.molcel.2021.08.008).

Exemplary CRISPR-Cas proteins and enzymes used in the prime editors herein include the following without limitation.

TABLE 5
Cas12a orthologs

| | | |
|---|---|---|
| KKP36646_ | MSNFFKNFTN LYELSKTLRF ELKPVGDTLT NMKDHLEYDE KLQTFLKDQN | (SEQ |
| (modified) | IDDAYQALKP QFDEIHEEFI TDSLESKKAK EIDESEYLDL FQEKKELNDS | ID |
| hypothetical | EKKLRNKIGE TENKAGEKWK KEKYPQYEWK KGSKIANGAD ILSCQDMLQF | NO: |
| protein | IKYKNPEDEK IKNYIDDTLK GFFTYFGGEN QNRANYYETK KEASTAVATR | 116) |
| UR27_C0015G0004 | IVHENLPKFC DNVIQFKHII KRKKDGTVEK TERKTEYLNA YQYLKNNNKI | |
| [Candidatus | TQIKDAETEK MIESTPIAEK IFDVYYFSSC LSQKQIEEYN RIIGHYNLLI | |
| Peregrinibacteria | NLYNQAKRSE GKHLSANEKK YKDLPKFKTL YKQIGCGKKK DLFYTIKCDT | |
| bacterium | EEEANKSRNE GKESHSVEEI INKAQEAINK YFKSNNDCEN INTVPDFINY | |
| GW2011_GWA | ILTKENYEGV YWSKAAMNTI SDKYFANYHD LQDRLKEAKV FQKADKKSED | |
| 2_33_10] | DIKIPEAIEL SGLFGVLDSL ADWQTTLFKS SILSNEDKLK IITDSQTPSE | |
| | ALLKMIENDI EKNMESELKE TNDIITLKKY KGNKEGTEKI KQWFDYTLAI | |
| | NRMLKYFLVK ENKIKGNSLD TNISEALKTL IYSDDAEWFK WYDALRNYLT | |
| | QKPQDEAKEN KLKLNEDNPS LAGGWDVNKE CSNFCVILKD KNEKKYLAIM | |
| | KKGENTLFQK EWTEGRGKNL TKKSNPLFEI NNCEILSKME YDFWADVSKM | |
| | IPKCSTQLKA VVNHFKQSDN EFIFPIGYKV TSGEKFREEC KISKQDFELN | |
| | NKVFNKNELS VTAMRYDLSS TQEKQYIKAF QKEYWELLFK QEKRDTKLTN | |
| | NEIFNEWINF CNKKYSELLS WERKYKDALT NWINFCKYFL SKYPKTTLEN | |
| | YSFKESENYN SLDEFYRDVD ICSYKLNINT TINKSILDRL VEEGKLYLFE | |
| | IKNQDSNDGK SIGHKNNLHT IYWNAIFENF DNRPKLNGEA EIFYRKAISK | |
| | DKLGIVKGKK TKNGTEIIKN YRESKEKFIL HVPITLNFCS NNEYVNDIVN | |
| | TKFYNFSNLH FLGIDRGEKH LAYYSLVNKN GEIVDQGTLN LPFTDKDGNQ | |
| | RSIKKEKYFY NKQEDKWEAK EVDCWNYNDL LDAMASNRDM ARKNWQRIGT | |
| | IKEAKNGYVS LVIRKIADLA VNNERPAFIV LEDLNTGEKR SRQKIDKSVY | |
| | QKFELALAKK LNFLVDKNAK RDEIGSPTKA LQLTPPVNNY GDIENKKQAG | |
| | IMLYTRANYT SQTDPATGWR KTIYLKAGPE ETTYKKDGKI KNKSVKDQII | |
| | ETFTDIGFDG KDYYFEYDKG EFVDEKTGEI KPKKWRLYSG ENGKSLDRER | |
| | GEREKDKYEW KIDKIDIVKI LDDLFVNEDK NISLLKQLKE GVELTRNNEH | |
| | GTGESLRFAI NLIQQIRNTG NNERDNDFIL SPVRDENGKH FDSREYWDKE | |
| | TKGEKISMPS SGDANGAFNI ARKGIIMNAH ILANSDSKDL SLFVSDEEWD | |
| | LHLNNKTEWK KQLNIFSSRK AMAKRKK | |
| KKR91555_ | MLFFMSTDIT NKPREKGVED NFTNLYEFSK TLTFGLIPLK WDDNKKMIVE | (SEQ |
| (modified) | DEDESVLRKY GVIEEDKRIA ESIKIAKFYL NILHRELIGK VLGSLKFEKK | ID |
| hypothetical | NLENYDRLLG EIEKNNKNEN ISEDKKKEIR KNFKKELSIA QDILLKKVGE | NO: |
| protein | VFESNGSGIL SSKNCLDELT KRFTRQEVDK LRRENKDIGV EYPDVAYREK | 117) |
| UU43_C0004G0 | DGKEETKSFF AMDVGYLDDF HKNRKQLYSV KGKKNSLGRR ILDNFEIFCK | |
| 003 | NKKLYEKYKN LDIDESEIER NENLTLEKVF DEDNYNERLT QEGLDEYAKI | |
| [Parcubacteria | LGGESNKQER TANIHGLNQI INLYIQKKQS EQKAEQKETG KKKIKENKKD | |
| (Falkowbacteria) | YPTFTCLQKQ ILSQVERKEI IIESDRDLIR ELKFFVEESK EKVDKARGII | |
| bacterium | EFLLNHEEND IDLAMVYLPK SKINSFVYKV FKEPQDELSV FQDGASNLDE | |
| GW2011_GWA | VSEDKIKTHL ENNKLTYKIF FKTLIKENHD FESFLILLQQ EIDLLIDGGE | |
| 2_41_14] | TVTLGGKKES ITSLDEKKNR LKEKLGWFEG KVRENEKMKD EEEGEFCSTV | |
| | LAYSQAVLNI TKRAEIFWLN EKQDAKVGED NKDMIFYKKF DEFADDGFAP | |
| | FFYFDKFGNY LKRRSRNTTK EIKLHFGNDD LLEGWDMNKE PEYWSFILRD | |
| | RNQYYLGIGK KDGEIFHKKL GNSVEAVKEA YELENEADFY EKIDYKQLNI | |
| | DRFEGIAFPK KTKTEEAFRQ VCKKRADEFL GGDTYEFKIL LAIKKEYDDF | |
| | KARRQKEKDW DSKFSKEKMS KLIEYYITCL GKRDDWKREN LNFRQPKEYE | |
| | DRSDFVRHIQ RQAYWIDPRK VSKDYVDKKV AEGEMFLFKV HNKDFYDFER | |
| | KSEDKKNHTA NLFTQYLLEL FSCENIKNIK SKDLIESIFE LDGKAEIRFR | |
| | PKTDDVKLKI YQKKGKDVTY ADKRDGNKEK EVIQHRRFAK DALTLHLKIR | |
| | LNFGKHVNLF DENKLVNTEL FAKVPVKILG MDRGENNLIY YCFLDEHGEI | |
| | ENGKCGSLNR VGEQIITLED DKKVKEPVDY FQLLVDREGQ RDWEQKNWQK | |
| | MTRIKDLKKA YLGNVVSWIS KEMLSGIKEG VVTIGVLEDL NSNEKRTRFF | |
| | RERQVYQGFE KALVNKLGYL VDKKYDNYRN VYQFAPIVDS VEEMEKNKQI | |
| | GTLVYVPASY TSKICPHPKC GWRERLYMKN SASKEKIVGL LKSDGIKISY | |
| | DQKNDRFYFE YQWEQEHKSD GKKKKYSGVD KVESNVSRMR WDVEQKKSID | |
| | FVDGTDGSIT NKLKSLLKGK GIELDNINQQ IVNQQKELGV EFFQSIIFYF | |
| | NLIMQIRNYD KEKSGSEADY IQCPSCLFDS RKPEMNGKLS AITNGDANGA | |
| | YNIARKGFMQ LCRIRENPQE PMKLITNREW DEAVREWDIY SAAQKIPVLS | |
| | EEN | |
| KDN25524_ | MLFQDFTHLY PLSKTVRFEL KPIDRTLEHI HAKNFLSQDE TMADMHQKVK | (SEQ |
| (modified) | VILDDYHRDF IADMMGEVKL TKLAEFYDVY LKERKNPKDD ELQKQLKDLQ | ID |
| hypothetical | AVLRKEIVKP IGNGGKYKAG YDRLFGAKLF KDGKELGDLA KEVIAQEGES | NO: |
| protein | SPKLAHLAHF EKFSTYFTGF HDNRKNMYSD EDKHTAIAYR LIHENLPRFI | 118) |
| MBO_03467 | DNLQILTTIK QKHSALYDQI INELTASGLD VSLASHLDGY HKLLTQEGIT | |
| [Moraxella | AYNTLLGGIS GEAGSPKIQG INELINSHHN QHCHKSERIA KLRPLHKQIL | |
| bovoculi 237] | SDGMSVSFLP SKFADDSEMC QAVNEFYRHY ADVFAKVQSL FDGEDDHQKD | |
| > WP_052585281.1 | GIYVEHKNLN ELSKQAFGDF ALLGRVLDGY YVDVVNPEEN ERFAKAKTDN | |
| type V CRISPR- | AKAKLTKEKD KFIKGVHSLA SLEQAIEHYT ARHDDESVQA GKLGQYFKHG | |
| associated | LAGVDNPIQK IHNNHSTIKG FLERERPAGE RALPKIKSGK NPEMTQLRQL | |
| protein Cpf1 | KELLDNALNV AHFAKLLTTK TTLDNQDGNF YGEFGVLYDE LAKIPTLYNK | |
| [Moraxella | VRDYLSQKPF STEKYKLNFG NPTLLNGWDL NKEKDNFGVI LQKDGCYYLA | |
| bovoculi] | LLDKAHKKVF DNAPNTGKSI YQKMIYKYLE VRKQFPKVFF SKEAIAINYH | |
| | PSKELVEIKD KGRQRSDDER LKLYRFILEC LKIHPKYDKK FEGAIGDIQL | |
| | FKKDKKGREV PISEKDLEDK INGIFSSKPK LEMEDFFIGE FKRYNPSQDL | |
| | VDQYNIYKKI DSNDNRKKEN FYNNHPKEKK DLVRYYYESM CKHEEWEESF | |
| | EFSKKLQDIG CYVDVNELFT EIETRRLNYK ISFCNINADY IDELVEQGQL | |
| | YLFQIYNKDF SPKAHGKPNL HTLYFKALFS EDNLADPIYK LNGEAQIFYR | |
| | KASLDMNETT IHRAGEVLEN KNPDNPKKRQ FVYDIIKDKR YTQDKEMLHV | |
| | PITMNFGVQG MTIKEFNKKV NQSIQQYDEV NVIGIDRGER HLLYLTVINS | |
| | KGEILEQCSL NDITTASANG TQMTTPYHKI LDKREIERLN ARVGWGEIET | |
| | IKELKSGYLS HVVHQISQLM LKYNAIVVLE DLNFGFKRGR FKVEKQIYQN | |
| | FENALIKKLN HLVLKDKADD EIGSYKNALQ LTNNFTDLKS IGKQTGELFY | |
| | VPAWNTSKID PETGFVDLLK PRYENIAQSQ AFFGKEDKIC YNADKDYFEF | |
| | HIDYAKFTDK AKNSRQIWTI CSHGDKRYVY DKTANQNKGA AKGINVNDEL | |
| | KSLFARHHIN EKQPNLVMDI CQNNDKEFHK SLMYLLKTLL ALRYSNASSD | |
| | EDFILSPVAN DEGVFENSAL ADDTQPQNAD ANGAYHIALK GLWLLNELKN | |
| | SDDLNKVKLA IDNQTWLNFA QNR | |
| KKT48220_ | MENIFDQFIG KYSLSKTLRF ELKPVGKTED FLKINKVFEK DQTIDDSYNQ | (SEQ |
| (modified) | AKFYFDSLHQ KFIDAALASD KTSELSFQNF ADVLEKQNKI ILDKKREMGA | ID |
| hypothetical | LRKRDKNAVG IDRLQKEIND AEDIIQKEKE KIYKDVRTLF DNEAESWKTY | NO: |
| protein | YQEREVDGKK ITFSKADLKQ KGADELTAAG ILKVLKYEFP EEKEKEFQAK | 119) |
| UW39_C0001G | NQPSLFVEEK ENPGQKRYIF DSFDKFAGYL TKFQQTKKNL YAADGTSTAV | |
| 0044 | ATRIADNFII FHQNTKVERD KYKNNHTDLG FDEENIFEIE RYKNCLLQRE | |
| [Parcubacteria | IEHIKNENSY NKIIGRINKK IKEYRDQKAK DTKLTKSDFP FFKNLDKQIL | |
| bacterium | GEVEKEKQLI EKTREKTEED VLIERFKEFI ENNEERFTAA KKLMNAFCNG | |
| GW2011_GWC2_ | EFESEYEGIY LKNKAINTIS RRWFVSDRDF ELKLPQQKSK NKSEKNEPKV | |
| 44_17] | KKFISIAEIK NAVEELDGDI FKAVFYDKKI IAQGGSKLEQ FLVIWKYEFE | |
| | YLFRDIEREN GEKLLGYDSC LKIAKQLGIF PQEKEAREKA TAVIKNYADA | |
| | GLGIFQMMKY FSLDDKDRKN TPGQLSTNFY AEYDGYYKDF EFIKYYNEFR | |
| | NFITKKPFDE DKIKLNFENG ALLKGWDENK EYDEMGVILK KEGRLYLGIM | |
| | HKNHRKLFQS MGNAKGDNAN RYQKMIYKQI ADASKDVPRL LLTSKKAMEK | |
| | FKPSQEILRI KKEKTFKRES KNFSLRDLHA LIEYYRNCIP QYSNWSFYDF | |
| | QFQDTGKYQN IKEFTDDVQK YGYKISFRDI DDEYINQALN EGKMYLFEVV | |
| | NKDIYNTKNG SKNLHTLYFE HILSAENLND PVFKLSGMAE IFQRQPSVNE | |
| | REKITTQKNQ CILDKGDRAY KYRRYTEKKI MFHMSLVLNT GKGEIKQVQF | |
| | NKIINQRISS SDNEMRVNVI GIDRGEKNLL YYSVVKQNGE IIEQASLNEI | |
| | NGVNYRDKLI EREKERLKNR QSWKPVVKIK DLKKGYISHV IHKICQLIEK | |
| | YSAIVVLEDL NMRFKQIRGG IERSVYQQFE KALIDKLGYL VFKDNRDLRA | |
| | PGGVLNGYQL SAPFVSFEKM RKQTGILFYT QAEYTSKTDP ITGFRKNVYI | |
| | SNSASLDKIK EAVKKEDAIG WDGKEQSYFF KYNPYNLADE KYKNSTVSKE | |
| | WAIFASAPRI RRQKGEDGYW KYDRVKVNEE FEKLLKVWNF VNPKATDIKQ | |
| | EIIKKEKAGD LQGEKELDGR LRNEWHSFIY LENLVLELRN SESLQIKIKA | |
| | GEVIAVDEGV DFIASPVKPF FTTPNPYIPS NLCWLAVENA DANGAYNIAR | |
| | KGVMILKKIR EHAKKDPEFK KLPNLFISNA EWDEAARDWG KYAGTTALNL | |
| | DH | |
| WP_031492824_ | MSSLTKFTNK YSKQLTIKNE LIPVGKTLEN IKENGLIDGD EQLNENYQKA | (SEQ |
| (modified) | KIIVDDELRD FINKALNNTQ IGNWRELADA LNKEDEDNIE KLQDKIRGII | ID |
| hypothetical | VSKFETEDLF SSYSIKKDEK IIDDDNDVEE EELDLGKKTS SFKYIFKKNL | NO: |
| protein | FKLVLPSYLK TTNQDKLKII SSEDNESTYF RGFFENRKNI FTKKPISTSI | 120) |
| [Succinivibrio | AYRIVHDNFP KELDNIRCEN VWQTECPQLI VKADNYLKSK NVIAKDKSLA | |
| dextrinosolvens] | NYFTVGAYDY FLSQNGIDFY NNIIGGLPAF AGHEKIQGLN EFINQECQKD | |
| | SELKSKLKNR HAFKMAVLFK QILSDREKSF VIDEFESDAQ VIDAVKNFYA | |
| | EQCKDNNVIF NLLNLIKNIA FLSDDELDGI FIEGKYLSSV SQKLYSDWSK | |
| | LRNDIEDSAN SKQGNKELAK KIKTNKGDVE KAISKYEFSL SELNSIVHDN | |
| | TKFSDLLSCT LHKVASEKLV KVNEGDWPKH LKNNEEKQKI KEPLDALLEI | |
| | YNTLLIENCK SENKNGNFYV DYDRCINELS SVVYLYNKTR NYCTKKPYNT | |
| | DKFKLNENSP QLGEGFSKSK ENDCLTLLFK KDDNYYVGII RKGAKINEDD | |
| | TQAIADNTDN CIFKMNYFLL KDAKKFIPKC SIQLKEVKAH FKKSEDDYIL | |
| | SDKEKFASPL VIKKSTELLA TAHVKGKKGN IKKFQKEYSK ENPTEYRNSL | |
| | NEWIAFCKEF LKTYKAATIF DITTLKKAEE YADIVEFYKD VDNLCYKLEF | |
| | CPIKTSFIEN LIDNGDLYLF RINNKDESSK STGTKNLHTL YLQAIFDERN | |
| | LNNPTIMLNG GAELFYRKES IEQKNRITHK AGSILVNKVC KDGTSLDDKI | |
| | RNEIYQYENK FIDTLSDEAK KVLPNVIKKE ATHDITKDKR FTSDKFFFHC | |
| | PLTINYKEGD TKQFNNEVLS FLRGNPDINI IGIDRGERNL IYVTVINQKG | |
| | EILDSVSENT VTNKSSKIEQ TVDYEEKLAV REKERIEAKR SWDSISKIAT | |
| | LKEGYLSAIV HEICLLMIKH NAIVVLENLN AGFKRIRGGL SEKSVYQKFE | |
| | KMLINKLNYF VSKKESDWNK PSGLLNGLQL SDQFESFEKL GIQSGFIFYV | |
| | PAAYTSKIDP TTGFANVLNL SKVRNVDAIK SFFSNENEIS YSKKEALFKF | |
| | SEDLDSLSKK GFSSFVKESK SKWNVYTEGE RIIKPKNKQG YREDKRINLT | |
| | FEMKKLLNEY KVSEDLENNL IPNLTSANLK DTFWKELFFI FKTTLQLRNS | |
| | VTNGKEDVLI SPVKNAKGEF FVSGTHNKTL PQDCDANGAY HIALKGLMIL | |
| | ERNNLVREEK DTKKIMAISN VDWFEYVQKR RGVL | |
| KKT50231_ | MKPVGKTEDF LKINKVFEKD QTIDDSYNQA KFYFDSLHQK FIDAALASDK | (SEQ |
| (modified) | TSELSFQNFA DVLEKQNKII LDKKREMGAL RKRDKNAVGI DRLQKEINDA | ID |
| hypothetical | EDIIQKEKEK IYKDVRTLED NEAESWKTYY QEREVDGKKI TFSKADLKQK | NO: |
| protein | GADFLTAAGI LKVLKYEFPE EKEKEFQAKN QPSLEVEEKE NPGQKRYIFD | 121) |
| UW40_C0007G | SFDKFAGYLT KFQQTKKNLY AADGTSTAVA TRIADNFIIF HQNTKVERDK | |
| 0006 | YKNNHTDLGF DEENIFEIER YKNCLLQREI EHIKNENSYN KIIGRINKKI | |
| [Parcubacteria | KEYRDQKAKD TKLTKSDFPF FKNLDKQILG EVEKEKQLIE KTREKTEEDV | |
| bacterium | LIERFKEFIE NNEERFTAAK KLMNAFCNGE FESEYEGIYL KNKAINTISR | |
| GW2011_GWF2_ | RWFVSDRDFE LKLPQQKSKN KSEKNEPKVK KFISIAEIKN AVEELDGDIF | |
| 44_17] | KAVFYDKKII AQGGSKLEQF LVIWKYEFEY LERDIERENG EKLLGYDSCL | |
| | KIAKQLGIFP QEKEAREKAT AVIKNYADAG LGIFQMMKYF SLDDKDRKNT | |
| | PGQLSTNFYA EYDGYYKDFE FIKYYNEFRN FITKKPFDED KIKLNFENGA | |
| | LLKGWDENKE YDFMGVILKK EGRLYLGIMH KNHRKLFQSM GNAKGDNANR | |
| | YQKMIYKQIA DASKDVPRLL LTSKKAMEKF KPSQEILRIK KEKTEKRESK | |
| | NESLRDLHAL IEYYRNCIPQ YSNWSFYDFQ FQDTGKYQNI KEFTDDVQKY | |
| | GYKISFRDID DEYINQALNE GKMYLFEVVN KDIYNTKNGS KNLHTLYFEH | |
| | ILSAENLNDP VFKLSGMAEI FQRQPSVNER EKITTQKNQC ILDKGDRAYK | |
| | YRRYTEKKIM FHMSLVLNTG KGEIKQVQEN KIINQRISSS DNEMRVNVIG | |
| | IDRGEKNLLY YSVVKQNGEI IEQASLNEIN GVNYRDKLIE REKERLKNRQ | |
| | SWKPVVKIKD LKKGYISHVI HKICQLIEKY SAIVVLEDLN MRFKQIRGGI | |
| | ERSVYQQFEK ALIDKLGYLV FKDNRDLRAP GGVLNGYQLS APFVSFEKMR | |
| | KQTGILFYTQ AEYTSKTDPI TGERKNVYIS NSASLDKIKE AVKKEDAIGW | |
| | DGKEQSYFFK YNPYNLADEK YKNSTVSKEW AIFASAPRIR RQKGEDGYWK | |
| | YDRVKVNEEF EKLLKVWNFV NPKATDIKQE IIKKEKAGDL QGEKELDGRL | |
| | RNFWHSFIYL ENLVLELRNS FSLQIKIKAG EVIAVDEGVD FIASPVKPFF | |
| | TTPNPYIPSN LCWLAVENAD ANGAYNIARK GVMILKKIRE HAKKDPEFKK | |
| | LPNLFISNAE WDEAARDWGK YAGTTALNLD H | |
| WP_004356401_ | MKVMENYQEF TNLFQLNKTL RFELKPIGKT CELLEEGKIF ASGSFLEKDK | (SEQ |
| (modified) | VRADNVSYVK KEIDKKHKIF IEETLSSFSI SNDLLKQYFD CYNELKAFKK | ID |
| hypothetical | DCKSDEEEVK KTALRNKCTS IQRAMREAIS QAFLKSPQKK LLAIKNLIEN | NO: |
| protein | VEKADENVQH FSEFTSYFSG FETNRENFYS DEEKSTSIAY RLVHDNLPIF | 122) |
| [Prevotella | IKNIYIFEKL KEQFDAKTLS EIFENYKLYV AGSSLDEVES LEYENNTLTQ | |
| disiens] | KGIDNYNAVI GKIVKEDKQE IQGLNEHINL YNQKHKDRRL PFFISLKKQI | |
| | LSDREALSWL PDMEKNDSEV IKALKGFYIE DGFENNVLTP LATLLSSLDK | |
| | YNLNGIFIRN NEALSSLSQN VYRNFSIDEA IDANAELQTF NNYELIANAL | |
| | RAKIKKETKQ GRKSFEKYEE YIDKKVKAID SLSIQEINEL VENYVSEENS | |
| | NSGNMPRKVE DYFSLMRKGD FGSNDLIENI KTKLSAAEKL LGTKYQETAK | |
| | DIFKKDENSK LIKELLDATK QFQHFIKPLL GTGEEADRDL VFYGDELPLY | |
| | EKFEELTLLY NKVRNRLTQK PYSKDKIRLC FNKPKLMTGW VDSKTEKSDN | |
| | GTQYGGYLFR KKNEIGEYDY FLGISSKAQL FRKNEAVIGD YERLDYYQPK | |
| | ANTIYGSAYE GENSYKEDKK RINKVIIAYI EQIKQTNIKK SIIESISKYP | |
| | NISDDDKVTP SSLLEKIKKV SIDSYNGILS FKSFQSVNKE VIDNLLKTIS | |
| | PLKNKAEFLD LINKDYQIFT EVQAVIDEIC KQKTFIYFPI SNVELEKEMG | |
| | DKDKPLCLFQ ISNKDLSFAK TFSANLRKKR GAENLHTMLF KALMEGNQDN | |
| | LDLGSGAIFY RAKSLDGNKP THPANEAIKC RNVANKDKVS LFTYDIYKNR | |
| | RYMENKELFH LSIVQNYKAA NDSAQLNSSA TEYIRKADDL HIIGIDRGER | |
| | NLLYYSVIDM KGNIVEQDSL NIIRNNDLET DYHDLLDKRE KERKANRQNW | |
| | EAVEGIKDLK KGYLSQAVHQ IAQLMLKYNA IIALEDLGQM FVTRGQKIEK | |
| | AVYQQFEKSL VDKLSYLVDK KRPYNELGGI LKAYQLASSI TKNNSDKQNG | |
| | FLFYVPAWNT SKIDPVTGFT DLLRPKAMTI KEAQDFFGAF DNISYNDKGY | |
| | FEFETNYDKF KIRMKSAQTR WTICTEGNRI KRKKDKNYWN YEEVELTEEF | |
| | KKLFKDSNID YENCNLKEEI QNKDNRKFFD DLIKLLQLTL QMRNSDDKGN | |
| | DYIISPVANA EGQFFDSRNG DKKLPLDADA NGAYNIARKG LWNIRQIKQT | |
| | KNDKKLNLSI SSTEWLDFVR EKPYLK | |
| CCB70584_ | MTNKFTNQYS LSKTLRFELI PQGKTLEFIQ EKGLLSQDKQ RAESYQEMKK | (SEQ |
| (modified) | TIDKFHKYFI DLALSNAKLT HLETYLELYN KSAETKKEQK FKDDLKKVQD | ID |
| Protein of | NLRKEIVKSF SDGDAKSIFA ILDKKELITV ELEKWFENNE QKDIYEDEKF | NO: |
| unknown | KTFTTYFTGF HQNRKNMYSV EPNSTAIAYR LIHENLPKEL ENAKAFEKIK | 123) |
| function | QVESLQVNFR ELMGEFGDEG LIFVNELEEM FQINYYNDVL SQNGITIYNS | |
| [Flavobacterium | IISGFTKNDI KYKGLNEYIN NYNQTKDKKD RLPKLKQLYK QILSDRISLS | |
| branchiophilum | FLPDAFTDGK QVLKAIFDFY KINLLSYTIE GQEESQNLLL LIRQTIENLS | |
| FL-15] | SFDTQKIYLK NDTHLTTISQ QVFGDESVES TALNYWYETK VNPKFETEYS | |
| | KANEKKREIL DKAKAVFTKQ DYFSIAFLQE VLSEYILTLD HTSDIVKKHS | |
| | SNCIADYFKN HFVAKKENET DKTEDFIANI TAKYQCIQGI LENADQYEDE | |
| | LKQDQKLIDN LKFFLDAILE LLHFIKPLHL KSESITEKDT AFYDVFENYY | |
| | EALSLLTPLY NMVRNYVTQK PYSTEKIKLN FENAQLLNGW DANKEGDYLT | |
| | TILKKDGNYE LAIMDKKHNK AFQKFPEGKE NYEKMVYKLL PGVNKMLPKV | |
| | FFSNKNIAYF NPSKELLENY KKETHKKGDT FNLEHCHTLI DFFKDSLNKH | |
| | EDWKYFDFQF SETKSYQDLS GFYREVEHQG YKINEKNIDS EYIDGLVNEG | |
| | KLFLFQIYSK DESPESKGKP NMHTLYWKAL FEEQNLQNVI YKLNGQAEIF | |
| | FRKASIKPKN IILHKKKIKI AKKHFIDKKT KTSEIVPVQT IKNLNMYYQG | |
| | KISEKELTQD DLRYIDNESI FNEKNKTIDI IKDKRFTVDK FQFHVPITMN | |
| | FKATGGSYIN QTVLEYLQNN PEVKIIGLDR GERHLVYLTL IDQQGNILKQ | |
| | ESLNTITDSK ISTPYHKLLD NKENERDLAR KNWGTVENIK ELKEGYISQV | |
| | VHKIATLMLE ENAIVVMEDL NFGFKRGRFK VEKQIYQKLE KMLIDKLNYL | |
| | VLKDKQPQEL GGLYNALQLT NKFESFQKMG KQSGELFYVP AWNTSKIDPT | |
| | TGFVNYFYTK YENVDKAKAF FEKFEAIREN AEKKYFEFEV KKYSDENPKA | |
| | EGTQQAWTIC TYGERIETKR QKDQNNKFVS TPINLTEKIE DFLGKNQIVY | |
| | GDGNCIKSQI ASKDDKAFFE TLLYWFKMTL QMRNSETRTD IDYLISPVMN | |
| | DNGTFYNSRD YEKLENPTLP KDADANGAYH IAKKGLMLLN KIDQADLTKK | |
| | VDLSISNRDW LQFVQKNK | |
| WP_005398606_ | MFEKLSNIVS ISKTIRFKLI PVGKTLENIE KLGKLEKDFE RSDFYPILKN | (SEQ |
| (modified) | ISDDYYRQYI KEKLSDLNLD WQKLYDAHEL LDSSKKESQK NLEMIQAQYR | ID |
| hypothetical | KVLFNILSGE LDKSGEKNSK DLIKNNKALY GKLFKKQFIL EVLPDFVNNN | NO: |
| protein | DSYSEEDLEG LNLYSKFTTR LKNEWETRKN VFTDKDIVTA IPFRAVNENE | 124) |
| [Helcococcus | GFYYDNIKIF NKNIEYLENK IPNLENELKE ADILDDNRSV KDYFTPNGEN | |
| kunzii] | YVITQDGIDV YQAIRGGFTK ENGEKVQGIN EILNLTQQQL RRKPETKNVK | |
| | LGVLTKLRKQ ILEYSESTSF LIDQIEDDND LVDRINKENV SFFESTEVSP | |
| | SLFEQIERLY NALKSIKKEE VYIDARNTQK FSQMLFGQWD VIRRGYTVKI | |
| | TEGSKEEKKK YKEYLELDET SKAKRYLNIR EIEELVNLVE GFEEVDVESV | |
| | LLEKFKMNNI ERSEFEAPIY GSPIKLEAIK EYLEKHLEEY HKWKLLLIGN | |
| | DDLDTDETFY PLLNEVISDY YIIPLYNLTR NYLTRKHSDK DKIKVNEDEP | |
| | TLADGWSESK ISDNRSIILR KGGYYYLGIL IDNKLLINKK NKSKKIYEIL | |
| | IYNQIPEFSK SIPNYPFTKK VKEHFKNNVS DFQLIDGYVS PLIITKEIYD | |
| | IKKEKKYKKD FYKDNNTNKN YLYTIYKWIE FCKQFLYKYK GPNKESYKEM | |
| | YDFSTLKDTS LYVNLNDFYA DVNSCAYRVL ENKIDENTID NAVEDGKLLL | |
| | FQIYNKDFSP ESKGKKNLHT LYWLSMESEE NLRTRKLKLN GQAEIFYRKK | |
| | LEKKPIIHKE GSILLNKIDK EGNTIPENIY HECYRYLNKK IGREDLSDEA | |
| | IALENKDVLK YKEARFDIIK DRRYSESQFF FHVPITENWD IKTNKNVNQI | |
| | VQGMIKDGEI KHIIGIDRGE RHLLYYSVID LEGNIVEQGS LNTLEQNRED | |
| | NSTVKVDYQN KLRTREEDRD RARKNWTNIN KIKELKDGYL SHVVHKLSRL | |
| | IIKYEAIVIM ENLNQGFKRG RFKVERQVYQ KFELALMNKL SALSFKEKYD | |
| | ERKNLEPSGI LNPIQACYPV DAYQELQGQN GIVFYLPAAY TSVIDPVTGF | |
| | TNLFRLKSIN SSKYEEFIKK FKNIYEDNEE EDFKFIFNYK DFAKANLVIL | |
| | NNIKSKDWKI STRGERISYN SKKKEYFYVQ PTEFLINKLK ELNIDYENID | |
| | IIPLIDNLEE KAKRKILKAL FDTFKYSVQL RNYDFENDYI ISPTADDNGN | |
| | NEDWINFIIS NGAFNIARKG LLLKDRIVNS NESKVDLKIK | |
| | YYNSNEIDID KTNLPNNGDA | |
| WP_021736722_ | MTQFEGFTNL YQVSKTLRFE LIPQGKTLKH IQEQGFIEED KARNDHYKEL | (SEQ |
| (modified) | KPIIDRIYKT YADQCLQLVQ LDWENLSAAI DSYRKEKTEE TRNALIEEQA | ID |
| CRISPR- | TYRNAIHDYF IGRTDNLTDA INKRHAEIYK GLFKAELENG KVLKQLGTVT | NO: |
| associated | TTEHENALLR SFDKFTTYFS GFYENRKNVF SAEDISTAIP HRIVQDNFPK | 125) |
| protein Cpf1, | FKENCHIFTR LITAVPSLRE HFENVKKAIG IFVSTSIEEV FSFPFYNQLL | |
| subtype | TQTQIDLYNQ LLGGISREAG TEKIKGLNEV LNLAIQKNDE TAHIIASLPH | |
| PREFRAN | RFIPLFKQIL SDRNTLSFIL EEFKSDEEVI QSFCKYKTLL RNENVLETAE | |
| [Acidaminococcus | ALFNELNSID LTHIFISHKK LETISSALCD HWDTLRNALY ERRISELTGK | |
| sp. BV3L6] | ITKSAKEKVQ RSLKHEDINL QEIISAAGKE LSEAFKQKTS EILSHAHAAL | |
| | DQPLPTTLKK QEEKEILKSQ LDSLLGLYHL LDWFAVDESN EVDPEFSARL | |
| | TGIKLEMEPS LSFYNKARNY ATKKPYSVEK FKLNFQMPTL ASGWDVNKEK | |
| | NNGAILFVKN GLYYLGIMPK QKGRYKALSF EPTEKTSEGF DKMYYDYFPD | |
| | AAKMIPKCST QLKAVTAHFQ THTTPILLSN NFIEPLEITK EIYDLNNPEK | |
| | EPKKFQTAYA KKTGDQKGYR EALCKWIDFT RDELSKYTKT TSIDLSSLRP | |
| | SSQYKDLGEY YAELNPLLYH ISFQRIAEKE IMDAVETGKL YLFQIYNKDE | |
| | AKGHHGKPNL HTLYWTGLES PENLAKTSIK LNGQAELFYR PKSRMKRMAH | |
| | RLGEKMLNKK LKDQKTPIPD TLYQELYDYV NHRLSHDLSD EARALLPNVI | |
| | TKEVSHEIIK DRRFTSDKFF FHVPITLNYQ AANSPSKENQ RVNAYLKEHP | |
| | ETPIIGIDRG ERNLIYITVI DSTGKILEQR SLNTIQQFDY QKKLDNREKE | |
| | RVAARQAWSV VGTIKDLKQG YLSQVIHEIV DLMIHYQAVV VLENLNFGFK | |
| | SKRTGIAEKA VYQQFEKMLI DKLNCLVLKD YPAEKVGGVL NPYQLTDQFT | |
| | SFAKMGTQSG FLFYVPAPYT SKIDPLTGFV DPFVWKTIKN HESRKHFLEG | |
| | FDFLHYDVKT GDFILHFKMN RNLSFQRGLP GEMPAWDIVE EKNETQFDAK | |
| | GTPFIAGKRI VPVIENHRFT GRYRDLYPAN ELIALLEEKG IVERDGSNIL | |
| | PKLLENDDSH AIDTMVALIR SVLQMRNSNA ATGEDYINSP VRDLNGVCED | |
| | SRFQNPEWPM DADANGAYHI ALKGQLLLNH LKESKDLKLQ NGISNQDWLA | |
| | YIQELRN | |
| WP_004339290_ | MSIYQEFVNK YSLSKTLRFE LIPQGKTLEN IKARGLILDD EKRAKDYKKA | (SEQ |
| (modified) | KQIIDKYHQF FIEEILSSVC ISEDLLQNYS DVYFKLKKSD DDNLQKDEKS | ID |
| hypothetical | AKDTIKKQIS KYINDSEKFK NLFNQNLIDA KKGQESDLIL WLKQSKDNGI | NO: |
| protein | ELFKANSDIT DIDEALEIIK SFKGWTTYFK GEHENRKNVY SSNDIPTSII | 126 |
| [Francisella | YRIVDDNLPK FLENKAKYES LKDKAPEAIN YEQIKKDLAE ELTEDIDYKT | |
| tularensis] | SEVNQRVFSL DEVFEIANEN NYLNQSGITK FNTIIGGKFV NGENTKRKGI | |
| | NEYINLYSQQ INDKTLKKYK MSVLFKQILS DTESKSFVID KLEDDSDVVT | |
| | TMQSFYEQIA AFKTVEEKSI KETLSLLEDD LKAQKLDLSK IYFKNDKSLT | |
| | DLSQQVEDDY SVIGTAVLEY ITQQVAPKNL DNPSKKEQDL IAKKTEKAKY | |
| | LSLETIKLAL EEENKHRDID KQCRFEEILS NFAAIPMIED EIAQNKDNLA | |
| | QISIKYQNQG KKDLLQASAE EDVKAIKDLL DQTNNLLHRL KIFHISQSED | |
| | KANILDKDEH FYLVFEECYF ELANIVPLYN KIRNYITQKP YSDEKFKLNE | |
| | ENSTLASGWD KNKESANTAI LFIKDDKYYL GIMDKKHNKI FSDKAIEENK | |
| | GEGYKKIVYK QIADASKDIQ NLMIIDGKTV CKKGRKDRNG VNRQLLSLKR | |
| | KHLPENIYRI KETKSYLKNE ARFSRKDLYD FIDYYKDRLD YYDFEFELKP | |
| | SNEYSDENDF TNHIGSQGYK LTFENISQDY INSLVNEGKL YLFQIYSKDE | |
| | SAYSKGRPNL HTLYWKALFD ERNLQDVVYK LNGEAELFYR KQSIPKKITH | |
| | PAKETIANKN KDNPKKESVF EYDLIKDKRF TEDKFFFHCP ITINFKSSGA | |
| | NKENDEINLL LKEKANDVHI LSIDRGERHL AYYTLVDGKG NIIKQDNENI | |
| | IGNDRMKTNY HDKLAAIEKD RDSARKDWKK INNIKEMKEG YLSQVVHEIA | |
| | KLVIEYNAIV VFEDLNFGFK RGRFKVEKQV YQKLEKMLIE KLNYLVEKDN | |
| | EFDKTGGVLR AYQLTAPFET FKKMGKQTGI IYYVPAGFTS KICPVTGFVN | |
| | QLYPKYESVS KSQEFFSKED KICYNLDKGY FEFSFDYKNF GDKAAKGKWT | |
| | IASFGSRLIN FRNSDKNHNW DTREVYPTKE LEKLLKDYSI EYGHGECIKA | |
| | AICGESDKKF FAKLTSVLNT ILQMRNSKTG TELDYLISPV ADVNGNEEDS | |
| | RQAPKNMPQD ADANGAYHIG LKGLMLLDRI KNNQEGKKLN LVIKNEEYFE | |
| | FVQNRNN | |
| WP_022501477 | MNKAADNYTG GNYDEFIALS KVQKTLRNEL KPTPFTAEHI KQRGIISEDE | (SEQ |
| type V CRISPR- | YRAQQSLELK KIADEYYRNY ITHKLNDINN LDFYNLEDAI EEKYKKNDKD | ID |
| associated | NRDKLDLVEK SKRGEIAKML SADDNEKSMF EAKLITKLLP DYVERNYTGE | NO: |
| protein Cpf1 | DKEKALETLA LFKGFTTYFK GYFKTRKNMF SGEGGASSIC HRIVNVNASI | 127) |
| [Eubacterium sp. | FYDNLKTEMR IQEKAGDEIA LIEEELTEKL DGWRLEHIFS RDYYNEVLAQ | |
| CAG: 76] | KGIDYYNQIC GDINKHMNLY CQQNKFKANI FKMMKIQKQI MGISEKAFEI | |
| | PPMYQNDEEV YASFNEFISR LEEVKLTDRL INILQNINIY NTAKIYINAR | |
| | YYTNVSSYVY GGWGVIDSAI ERYLYNTIAG KGQSKVKKIE NAKKDNKEMS | |
| | VKELDSIVAE YEPDYENAPY IDDDDNAVKA FGGQGVLGYF NKMSELLADV | |
| | SLYTIDYNSD DSLIENKESA LRIKKQLDDI MSLYHWLQTF IIDEVVEKDN | |
| | AFYAELEDIC CELENVVTLY DRIRNYVTKK PYSTQKFKLN FASPTLAAGW | |
| | SRSKEFDNNA IILLRNNKYY IAIFNVNNKP DKQIIKGSEE QRLSTDYKKM | |
| | VYNLLPGPNK MLPKVFIKSD TGKRDYNPSS YILEGYEKNR HIKSSGNEDI | |
| | NYCHDLIDYY KACINKHPEW KNYGFKFKET NQYNDIGQFY KDVEKQGYSI | |
| | SWAYISEEDI NKLDEEGKIY LFEIYNKDLS AHSTGRDNLH TMYLKNIFSE | |
| | DNLKNICIEL NGEAELFYRK SSMKSNITHK KDTILVNKTY INETGVRVSL | |
| | SDEDYMKVYN YYNNNYVIDT ENDKNLIDII EKIGHRKSKI DIVKDKRYTE | |
| | DKYFLYLPIT INYGIEDENV NSKIIEYIAK QDNMNVIGID RGERNLIYIS | |
| | VIDNKGNIIE QKSENLVNNY DYKNKLKNME KTRDNARKNW QEIGKIKDVK | |
| | SGYLSGVISK IARMVIDYNA IIVMEDLNKG FKRGREKVER QVYQKFENML | |
| | ISKLNYLVFK ERKADENGGI LRGYQLTYIP KSIKNVGKQC GCIFYVPAAY | |
| | TSKIDPATGF INIFDFKKYS GSGINAKVKD KKEFLMSMNS IRYINECSEE | |
| | YEKIGHRELF AFSFDYNNFK TYNVSSPVNE WTAYTYGERI KKLYKDGRWL | |
| | RSEVLNLTEN LIKLMEQYNI EYKDGHDIRE DISHMDETRN ADFICSLFEE | |
| | LKYTVQLRNS KSEAEDENYD RLVSPILNSS NGFYDSSDYM ENENNTTHTM | |
| | PKDADANGAY CIALKGLYEI NKIKQNWSDD KKFKENELYI NVTEWLDYIQ | |
| | NRRFE | |
| WP_014550095 | MSIYQEFVNK YSLSKTLRFE LIPQGKTLEN IKARGLILDD EKRAKDYKKA | (SEQ |
| type V CRISPR- | KQIIDKYHQF FIEEILSSVC ISEDLLQNYS DVYFKLKKSD DDNLQKDEKS | ID |
| associated | AKDTIKKQIS EYIKDSEKFK NLFNQNLIDA KKGQESDLIL WLKQSKDNGI | NO: |
| protein Cpf1 | ELFKANSDIT DIDEALEIIK SFKGWTTYFK GFHENRKNVY SSNDIPTSII | 128) |
| [Francisella | YRIVDDNLPK FLENKAKYES LKDKAPEAIN YEQIKKDLAE ELTEDIDYKT | |
| tularensis] | SEVNQRVESL DEVFEIANEN NYLNQSGITK FNTIIGGKFV NGENTKRKGI | |
| | NEYINLYSQQ INDKTLKKYK MSVLFKQILS DTESKSFVID KLEDDSDVVT | |
| | TMQSFYEQIA AFKTVEEKSI KETLSLLEDD LKAQKLDLSK IYFKNDKSLT | |
| | DLSQQVEDDY SVIGTAVLEY ITQQVAPKNL DNPSKKEQDL IAKKTEKAKY | |
| | LSLETIKLAL EEENKHRDID KQCRFEEILA NFAAIPMIED EIAQNKDNLA | |
| | QISIKYQNQG KKDLLQASAE DDVKAIKDLL DQTNNLLHRL KIFHISQSED | |
| | KANILDKDEH FYLVFEECYF ELANIVPLYN KIRNYITQKP YSDEKFKLNE | |
| | ENSTLANGWD KNKEPDNTAI LFIKDDKYYL GVMNKKNNKI FDDKAIKENK | |
| | GEGYKKIVYK LLPGANKMLP KVFFSAKSIK FYNPSEDILR IRNHSTHTKN | |
| | GNPQKGYEKF EFNIEDCRKF IDFYKESISK HPEWKDEGER FSDTQRYNSI | |
| | DEFYREVENQ GYKLTFENIS ESYIDSVVNQ GKLYLFQIYN KDESAYSKGR | |
| | PNLHTLYWKA LEDERNLQDV VYKLNGEAEL FYRKKSIPKK ITHPAKEAIA | |
| | NKNKDNPKKE SFFEYDLIKD KRFTEDKFFF HCPITINEKS SGANKENDEI | |
| | NLLLKEKAND VHILSIDRGE RHLAYYTLVD GKGNIIKQDT ENIIGNDRMK | |
| | TNYHDKLAAI EKDRDSARKD WKKINNIKEM KEGYLSQVVH EIAKLVIEHN | |
| | AIVVFEDLNF GFKRGRFKVE KQVYQKLEKM LIEKLNYLVF KDNEEDKTGG | |
| | VLRAYQLTAP FETFKKMGKQ TGIIYYVPAG FTSKICPVTG FVNQLYPKYE | |
| | SVSKSQEFFS KEDKICYNLD KGYFEFSEDY KNFGDKAAKG KWTIASFGSR | |
| | LINERNSDKN HNWDTREVYP TKELEKLLKD YSIEYGHGEC IKAAICGESD | |
| | KKFFAKLTSI LNTILQMRNS KTGTELDYLI SPVADVNGNF FDSRQAPKNM | |
| | PQDADANGAY HIGLKGLMLL DRIKNNQEGK KLNLVIKNEE YFEFVQNRNN | |
| WP_003034647 | MSIYQEFVNK YSLSKTLRFE LIPQGKTLEN IKARGLILDD EKRAKDYKKA | (SEQ |
| type V CRISPR- | KQIIDKYHQF FIEEILSSVC ISEDLLQNYS DVYFKLKKSD DDNLQKDEKS | ID |
| associated | AKDTIKKQIS EYIKDSEKFK NLFNQNLIDA KKGQESDLIL WLKQSKDNGI | NO: |
| protein Cpf1 | ELFKANSDIT DIDEALEIIK SFKGWTTYFK GEHENRKNVY SSDDIPTSII | 129) |
| [Francisella | YRIVDDNLPK FLENKAKYES LKDKAPEAIN YEQIKKDLAE ELTEDIDYKT | |
| tularensis] | SEVNQRVFSL DEVFEIANEN NYLNQSGITK FNTIIGGKFV NGENTKRKGI | |
| | NEYINLYSQQ INDKTLKKYK MSVLFKQILS DTESKSFVID KLEDDSDVVT | |
| | TMQSFYEQIA AFKTVEEKSI KETLSLLEDD LKAQKLDLSK IYFKNDKSLT | |
| | DLSQQVEDDY SVIGTAVLEY ITQQVAPKNL DNPSKKEQDL IAKKTEKAKY | |
| | LSLETIKLAL EEFNKHRDID KQCRFEEILA NFAAIPMIED EIAQNKDNLA | |
| | QISLKYQNQG KKDLLQASAE EDVKAIKDLL DQTNNLLHRL KIFHISQSED | |
| | KANILDKDEH FYLVFEECYF ELANIVPLYN KIRNYITQKP YSDEKFKLNF | |
| | ENSTLANGWD KNKEPDNTAI LFIKDDKYYL GVMNKKNNKI FDDKAIKENK | |
| | GEGYKKIVYK LLPGANKMLP KVFFSAKSIK FYNPSEDILR IRNHSTHTKN | |
| | GNPQKGYEKF EFNIEDCRKF IDFYKESISK HPEWKDEGER FSDTQRYNSI | |
| | DEFYREVENQ GYKLTFENIS ESYIDSVVNQ GKLYLFQIYN KDESAYSKGR | |
| | PNLHTLYWKA LEDERNLQDV VYKLNGEAEL FYRKQSIPKK ITHPAKEAIA | |
| | NKNKDNPKKE SVFEYDLIKD KRFTEDKFFF HCPITINEKS SGANKENDEI | |
| | NLLLKEKAND VHILSIDRGE RHLAYYTLVD GKGNIIKQDT ENIIGNDRMK | |
| | TNYHDKLAAI EKDRDSARKD WKKINNIKEM KEGYLSQVVH EIAKLVIEHN | |
| | AIVVFEDLNF GFKRGRFKVE KQVYQKLEKM LIEKLNYLVF KDNEEDKTGG | |
| | VLRAYQLTAP FETFKKMGKQ TGIIYYVPAG FTSKICPVTG FVNQLYPKYE | |
| | SVSKSQEFFS KEDKICYNLD KGYFEFSEDY KNFGDKAAKG KWTIASFGSR | |
| | LINFRNSDKN HNWDTREVYP TKELEKLLKD YSIEYGHGEC IKAAICGESD | |
| | KKFFAKLTSV LNTILQMRNS KTGTELDYLI SPVADVNGNF FDSRQAPKNM | |
| | PQDADANGAY HIGLKGLMLL DRIKNNQEGK KLNLVIKNEE YFEFVQNRNN | |
| WP_003040289.1 | MSIYQEFVNK YSLSKTLRFE LIPQGKTLEN IKARGLILDD EKRAKDYKKA | (SEQ |
| type V CRISPR- | KQIIDKYHQF FIEEILSSVC ISEDLLQNYS DVYFKLKKSD DDNLQKDEKS | ID |
| associated | AKDTIKKQIS EYIKDSEKFK NLFNQNLIDA KKGQESDLIL WLKQSKDNGI | NO: |
| protein Cpf1 | ELFKANSDIT DIDEALEIIK SFKGWTTYFK GFHENRKNVY SSNDIPTSII | 130) |
| [Francisella | YRIVDDNLPK FLENKAKYES LKDKAPEAIN YEQIKKDLAE ELTFDIDYKT | |
| tularensis subsp. | SEVNQRVESL DEVFEIANEN NYLNQSGITK FNTIIGGKFV NGENTKRKGI | |
| novicida U112] | NEYINLYSQQ INDKTLKKYK MSVLFKQILS DTESKSFVID KLEDDSDVVT | |
| | TMQSFYEQIA AFKTVEEKSI KETLSLLEDD LKAQKLDLSK IYFKNDKSLT | |
| | DLSQQVEDDY SVIGTAVLEY ITQQIAPKNL DNPSKKEQEL IAKKTEKAKY | |
| | LSLETIKLAL EEENKHRDID KQCRFEEILA NFAAIPMIED EIAQNKDNLA | |
| | QISIKYQNQG KKDLLQASAE DDVKAIKDLL DQTNNLLHKL KIFHISQSED | |
| | KANILDKDEH FYLVFEECYF ELANIVPLYN KIRNYITQKP YSDEKFKLNF | |
| | ENSTLANGWD KNKEPDNTAI LFIKDDKYYL GVMNKKNNKI FDDKAIKENK | |
| | GEGYKKIVYK LLPGANKMLP KVFFSAKSIK FYNPSEDILR IRNHSTHTKN | |
| | GSPQKGYEKF EFNIEDCRKE IDFYKQSISK HPEWKDEGER FSDTQRYNSI | |
| | DEFYREVENQ GYKLTFENIS ESYIDSVVNQ GKLYLFQIYN KDESAYSKGR | |
| | PNLHTLYWKA LEDERNLQDV VYKLNGEAEL FYRKQSIPKK ITHPAKEAIA | |
| | NKNKDNPKKE SVFEYDLIKD KRFTEDKFFF HCPITINFKS SGANKENDEI | |
| | NLLLKEKAND VHILSIDRGE RHLAYYTLVD GKGNIIKQDT FNIIGNDRMK | |
| | TNYHDKLAAI EKDRDSARKD WKKINNIKEM KEGYLSQVVH EIAKLVIEYN | |
| | AIVVFEDLNE GFKRGRFKVE KQVYQKLEKM LIEKLNYLVF KDNEEDKTGG | |
| | VLRAYQLTAP FETFKKMGKQ TGIIYYVPAG FTSKICPVTG FVNQLYPKYE | |
| | SVSKSQEFFS KEDKICYNLD KGYFEFSEDY KNFGDKAAKG KWTIASFGSR | |
| | LINERNSDKN HNWDTREVYP TKELEKLLKD YSIEYGHGEC IKAAICGESD | |
| | KKFFAKLTSV LNTILQMRNS KTGTELDYLI SPVADVNGNF FDSRQAPKNM | |
| | PQDADANGAY HIGLKGLMLL GRIKNNQEGK KLNLVIKNEE YFEFVQNRNN | |
| KKQ38174 | MKSFDSFTNL YSLSKTLKFE MRPVGNTQKM LDNAGVFEKD KLIQKKYGKT | (SEQ |
| hypothetical | KPYFDRLHRE FIEEALTGVE LIGLDENERT LVDWQKDKKN NVAMKAYENS | ID |
| protein | LQRLRTEIGK IFNLKAEDWV KNKYPILGLK NKNTDILFEE AVFGILKARY | NO: |
| US54_C0016G0 | GEEKDTFIEV EEIDKTGKSK INQISIFDSW KGFTGYFKKF FETRKNFYKN | 131) |
| 015 [Candidatus | DGTSTAIATR IIDQNLKRFI DNLSIVESVR QKVDLAETEK SFSISLSQFF | |
| Roizmanbacteria | SIDFYNKCLL QDGIDYYNKI IGGETLKNGE KLIGLNELIN QYRQNNKDQK | |
| bacterium | IPFFKLLDKQ ILSEKILFLD EIKNDTELIE ALSQFAKTAE EKTKIVKKLE | |
| GW2011_GWA | ADFVENNSKY DLAQIYISQE AFNTISNKWT SETETFAKYL FEAMKSGKLA | |
| 2_37_7] | KYEKKDNSYK FPDFIALSQM KSALLSISLE GHFWKEKYYK ISKFQEKTNW | |
| | EQFLAIFLYE ENSLESDKIN TKDGETKQVG YYLFAKDLHN LILSEQIDIP | |
| | KDSKVTIKDF ADSVLTIYQM AKYFAVEKKR AWLAEYELDS TYTQPDTGYL | |
| | QFYDNAYEDI VQVYNKLRNY LTKKPYSEEK WKLNFENSTL ANGWDKNKES | |
| | DNSAVILQKG GKYYLGLITK GHNKIFDDRF QEKFIVGIEG GKYEKIVYKF | |
| | FPDQAKMFPK VCFSAKGLEF FRPSEEILRI YNNAEFKKGE TYSIDSMQKL | |
| | IDFYKDCLTK YEGWACYTER HLKPTEEYQN NIGEFERDVA EDGYRIDEQG | |
| | ISDQYIHEKN EKGELHLFEI HNKDWNLDKA RDGKSKTTQK NLHTLYFESL | |
| | FSNDNVVQNF PIKLNGQAEI FYRPKTEKDK LESKKDKKGN KVIDHKRYSE | |
| | NKIFFHVPLT LNRTKNDSYR FNAQINNFLA NNKDINIIGV DRGEKHLVYY | |
| | SVITQASDIL ESGSLNELNG VNYAEKLGKK AENREQARRD WQDVQGIKDL | |
| | KKGYISQVVR KLADLAIKHN AIIILEDLNM RFKQVRGGIE KSIYQQLEKA | |
| | LIDKLSFLVD KGEKNPEQAG HLLKAYQLSA PFETFQKMGK QTGIIFYTQA | |
| | SYTSKSDPVT GWRPHLYLKY FSAKKAKDDI AKFTKIEFVN DRFELTYDIK | |
| | DEQQAKEYPN KTVWKVCSNV ERFRWDKNLN QNKGGYTHYT NITENIQELF | |
| | TKYGIDITKD LLTQISTIDE KQNTSFFRDF IFYFNLICQI RNTDDSEIAK | |
| | KNGKDDFILS PVEPFFDSRK DNGNKLPENG DDNGAYNIAR KGIVILNKIS | |
| | QYSEKNENCE KMKWGDLYVS NIDWDNFVTQ ANARH | |
| WP_022097749 | MNGNRSIVYR EFVGVTPVAK TLRNELRPVG HTQEHIIQNG LIQEDELRQE | (SEQ |
| type V CRISPR- | KSTELKNIMD DYYREYIDKS LSGLTDLDFT LLFELMNSVQ SSLSKDNKKA | ID |
| associated | LEKEHNKMRE QICTHLQSDS DYKNMFNAKL FKEILPDFIK NYNQYDVKDK | NO: |
| protein Cpf1 | AGKLETLALF NGFSTYFTDF FEKRKNVFTK EAVSTSIAYR IVHENSLIFL | 132) |
| [Eubacterium | ANMTSYKKIS EKALDEIEVI EKNNQDKMGD WELNQIFNPD FYNMVLIQSG | |
| eligens CAG: 72] | IDFYNEICGV VNAHMNLYCQ QTKNNYNLFK MRKLHKQILA YTSTSFEVPK | |
| | MFEDDMSVYN AVNAFIDETE KGNIIGKLKD IVNKYDELDE KRIYISKDFY | |
| | ETLSCFMSGN WNLITGCVEN FYDENIHAKG KSKEEKVKKA VKEDKYKSIN | |
| | DVNDLVEKYI DEKERNEFKN SNAKQYIREI SNIITDTETA HLEYDEHISL | |
| | IESEEKADEI KKRLDMYMNM YHWVKAFIVD EVLDRDEMFY SDIDDIYNIL | |
| | ENIVPLYNRV RNYVTQKPYT SKKIKLNFQS PTLANGWSQS KEFDNNAIIL | |
| | IRDNKYYLAI FNAKNKPDKK IIQGNSDKKN DNDYKKMVYN LLPGANKMLP | |
| | KVFLSKKGIE TFKPSDYIIS GYNAHKHIKT SENFDISFCR DLIDYFKNSI | |
| | EKHAEWRKYE FKFSATDSYN DISEFYREVE MQGYRIDWTY ISEADINKLD | |
| | EEGKIYLFQI YNKDFAENST GKENLHTMYF KNIFSEENLK NIVIKINGQA | |
| | ELFYRKASVK NPVKHKKDSV LVNKTYKNQL DNGDVVRIPI PDDIYNEIYK | |
| | MYNGYIKESD LSEAAKEYLD KVEVRTAQKD IVKDYRYTVD KYFIHTPITI | |
| | NYKVTARNNV NDMAVKYIAQ NDDIHVIGID RGERNLIYIS VIDSHGNIVK | |
| | QKSYNILNNY DYKKKLVEKE KTREYARKNW KSIGNIKELK EGYISGVVHE | |
| | IAMLMVEYNA IIAMEDLNYG FKRGREKVER QVYQKFESML INKLNYFASK | |
| | GKSVDEPGGL LKGYQLTYVP DNIKNLGKQC GVIFYVPAAF TSKIDPSTGE | |
| | ISAFNFKSIS TNASRKQFFM QFDEIRYCAE KDMFSFGFDY NNEDTYNITM | |
| | GKTQWTVYTN GERLQSEENN ARRTGKTKSI NLTETIKLLL EDNEINYADG | |
| | HDVRIDMEKM YEDKNSEFFA QLLSLYKLTV QMRNSYTEAE EQEKGISYDK | |
| | IISPVINDEG EFFDSDNYKE SDDKECKMPK DADANGAYCI ALKGLYEVLK | |
| | IKSEWTEDGE DRNCLKLPHA EWLDFIQNKR YE | |
| WP_021739647 | MIKKTIDTVL NVRPIFVGIQ HLYFYEGPCR FGEGDELMPE YDAMMNQEMN | (SEQ |
| hypothetical | AAYVNEVVQH ETEGVHIMDP IYVERDDWER SPEAMYEKMA EDIDKVDFYL | ID |
| protein | FHFGIGRGDI YLEFAERYKK PVGAAPGLCC DGIGNTAAVK NRGLEAYAFM | NO: |
| [Eubacterium | SWDEFDTWMR VLRVRKCLKN TRVLLAVRWD SNRSYSSYDN FINQSDVTNK | 133) |
| ramulus] | WGIQFRHVNV HELLDQTHPV DPTTNPSTPG RKALNINDED MKEIEKITDE | |
| | LIANAEACTM EPDMVKKTIQ AYYTVQKLLD AYDCNAFTAP CPDLCSTRRE | |
| | SEEKFTLCMT HSLNDENGIS SACEYDINSV IGKVIMTNLS GKAPYMGNTN | |
| | AIVEDKEGHM IPFHKENDNT IEDIADKTNL YMTFHSTPNR NLKGLKAEKE | |
| | RYRLAPFAYS GFGATIRYDF AQDIGQVITM IRISPDATKI FIAKGTISGG | |
| | AGYEMKNCDQ GVFFNVADKV DFYHKQQYFG NHTVLAYGDY VEELKMLAEA | |
| | LGIEAVIA | |
| gi\|800943167 | MKNFSNLYQV SKTVRFELKP IGNTLENIKN KSLLKNDSIR AESYQKMKKT | (SEQ |
| WP_045971446.1 | IDEFHKYFID LALNNKKLSY LNEYIALYTQ SAEAKKEDKF KADFKKVQDN | ID |
| type V CRISPR- | LRKEIVSSFT EGEAKAIFSV LDKKELITIE LEKWKNENNL AVYLDESEKS | NO: |
| associated | FTTYFTGFHQ NRKNMYSAEA NSTAIAYRLI HENLPKFIEN SKAFEKSSQI | 134) |
| protein Cpf1 | AELQPKIEKL YKEFEAYLNV NSISELFEID YENEVLTQKG ITVYNNIIGG | |
| [Flavobacterium | RTATEGKQKI QGLNEIINLY NQTKPKNERL PKLKQLYKQI LSDRISLSEL | |
| sp. 316] | PDAFTEGKQV LKAVFEFYKI NLLSYKQDGV EESQNLLELI QQVVKNLGNQ | |
| | DVNKIYLKND TSLTTIAQQL FGDESVESAA LQYRYETVVN PKYTAEYQKA | |
| | NEAKQEKLDK EKIKFVKQDY FSIAFLQEVV ADYVKTLDEN LDWKQKYTPS | |
| | CIADYFTTHF IAKKENEADK TENFIANIKA KYQCIQGILE QADDYEDELK | |
| | QDQKLIDNIK FFLDAILEVV HFIKPLHLKS ESITEKDNAF YDVFENYYEA | |
| | LNVVTPLYNM VRNYVTQKPY STEKIKLNFE NAQLLNGWDA NKEKDYLTTI | |
| | LKRDGNYFLA IMDKKHNKTF QQFTEDDENY EKIVYKLLPG VNKMLPKVFF | |
| | SNKNIAFFNP SKEILDNYKN NTHKKGATEN LKDCHALIDF FKDSLNKHED | |
| | WKYFDFQFSE TKTYQDLSGF YKEVEHQGYK INFKKVSVSQ IDTLIEEGKM | |
| | YLFQIYNKDF SPYAKGKPNM HTLYWKALFE TQNLENVIYK LNGQAEIFFR | |
| | KASIKKKNII THKAHQPIAA KNPLTPTAKN TFAYDLIKDK RYTVDKFQFH | |
| | VPITMNFKAT GNSYINQDVL AYLKDNPEVN IIGLDRGERH LVYLTLIDQK | |
| | GTILLQESLN VIQDEKTHTP YHTLLDNKEI ARDKARKNWG SIESIKELKE | |
| | GYISQVVHKI TKMMIEHNAI VVMEDLNFGF KRGREKVEKQ IYQKLEKMLI | |
| | DKLNYLVLKD KQPHELGGLY NALQLTNKFE SFQKMGKQSG FLFYVPAWNT | |
| | SKIDPTTGFV NYFYTKYENV EKAKTFFSKF DSILYNKTKG YFEFVVKNYS | |
| | DENPKAADTR QEWTICTHGE RIETKRQKEQ NNNFVSTTIQ LTEQFVNFFE | |
| | KVGLDLSKEL KTQLIAQNEK SFFEELFHLL KLTLQMRNSE SHTEIDYLIS | |
| | PVANEKGIFY DSRKATASLP IDADANGAYH IAKKGLWIME QINKTNSEDD | |
| | LKKVKLAISN REWLQYVQQV QKK | |
| WP_044110123.1 | MKQFTNLYQL SKTLRFELKP IGKTLEHINA NGFIDNDAHR AESYKKVKKL | (SEQ |
| type V CRISPR- | IDDYHKDYIE NVLNNFKLNG EYLQAYFDLY SQDTKDKQFK DIQDKLRKSI | ID |
| associated | ASALKGDDRY KTIDKKELIR QDMKTFLKKD TDKALLDEFY EFTTYFTGYH | NO: |
| protein Cpfl | ENRKNMYSDE AKSTAIAYRL IHDNLPKFID NIAVFKKIAN TSVADNESTI | 135) |
| [Prevotella | YKNFEEYLNV NSIDEIFSLD YYNIVLTQTQ IEVYNSIIGG RTLEDDTKIQ | |
| brevis] | GINEFVNLYN QQLANKKDRL PKLKPLFKQI LSDRVQLSWL QEEENTGADV | |
| | LNAVKEYCTS YFDNVEESVK VLLTGISDYD LSKIYITNDL ALTDVSQRME | |
| | GEWSIIPNAI EQRLRSDNPK KTNEKEEKYS DRISKLKKLP KSYSLGYINE | |
| | CISELNGIDI ADYYATLGAI NTESKQEPSI PTSIQVHYNA LKPILDTDYP | |
| | REKNLSQDKL TVMQLKDLLD DFKALQHFIK PLLGNGDEAE KDEKFYGELM | |
| | QLWEVIDSIT PLYNKVRNYC TRKPFSTEKI KVNFENAQLL DGWDENKEST | |
| | NASIILRKNG MYYLGIMKKE YRNILTKPMP SDGDCYDKVV YKFFKDITTM | |
| | VPKCTTQMKS VKEHFSNSND DYTLFEKDKF IAPVVITKEI FDLNNVLYNG | |
| | VKKFQIGYLN NTGDSFGYNH AVEIWKSFCL KFLKAYKSTS IYDFSSIEKN | |
| | IGCYNDLNSF YGAVNLLLYN LTYRKVSVDY IHQLVDEDKM YLFMIYNKDF | |
| | STYSKGTPNM HTLYWKMLED ESNLNDVVYK LNGQAEVFYR KKSITYQHPT | |
| | HPANKPIDNK NVNNPKKQSN FEYDLIKDKR YTVDKEMFHV PITLNFKGMG | |
| | NGDINMQVRE YIKTTDDLHE IGIDRGERHL LYICVINGKG EIVEQYSLNE | |
| | IVNNYKGTEY KTDYHTLLSE RDKKRKEERS SWQTIEGIKE LKSGYLSQVI | |
| | HKITQLMIKY NAIVLLEDLN MGFKRGRQKV ESSVYQQFEK ALIDKLNYLV | |
| | DKNKDANEIG GLLHAYQLTN DPKLPNKNSK QSGELFYVPA WNTSKIDPVT | |
| | GFVNLLDTRY ENVAKAQAFF KKEDSIRYNK EYDRFEFKED YSNFTAKAED | |
| | TRTQWTLCTY GTRIETERNA EKNSNWDSRE IDLTTEWKTL FTQHNIPLNA | |
| | NLKEAILLQA NKNFYTDILH LMKLTLQMRN SVTGTDIDYM VSPVANECGE | |
| | FFDSRKVKEG LPVNADANGA YNIARKGLWL AQQIKNANDL SDVKLAITNK | |
| | EWLQFAQKKQ YLKD | |
| WP_036388671.1 | MLFQDFTHLY PLSKTMRFEL KPIGKTLEHI HAKNFLSQDE TMADMYQKVK | (SEQ |
| type V CRISPR- | AILDDYHRDF IADMMGEVKL TKLAEFYDVY LKERKNPKDD GLQKQLKDLQ | ID |
| associated | AVLRKEIVKP IGNGGKYKAG YDRLFGAKLF KDGKELGDLA KEVIAQEGES | NO: |
| protein Cpf1 | SPKLAHLAHF EKFSTYFTGF HDNRKNMYSD EDKHTAITYR LIHENLPRFI | 136) |
| [Moraxella | DNLQILATIK QKHSALYDQI INELTASGLD VSLASHLDGY HKLLTQEGIT | |
| caprae] | AYNTLLGGIS GEAGSRKIQG INELINSHHN QHCHKSERIA KLRPLHKQIL | |
| | SDGMGVSFLP SKFADDSEMC QAVNEFYRHY ADVFAKVQSL EDGEDDHQKD | |
| | GIYVEHKNLN ELSKQAFGDF ALLGRVLDGY YVDVVNPEEN ERFAKAKTDN | |
| | AKAKLTKEKD KFIKGVHSLA SLEQAIEHYT ARHDDESVQA GKLGQYFKHG | |
| | LAGVDNPIQK IHNNHSTIKG FLERERPAGE RALPKIKSGK NPEMTQLRQL | |
| | KELLDNALNV AHFAKLLTTK TTLDNQDGNF YGEFGALYDE LAKIPTLYNK | |
| | VRDYLSQKPF STEKYKLNFG NPTLLNGWDL NKEKDNFGII LQKDGCYYLA | |
| | LLDKAHKKVF DNAPNTGKNV YQKMIYKLLP GPNKMLPKVF FAKSNLDYYN | |
| | PSAELLDKYA QGTHKKGNNF NLKDCHALID FFKAGINKHP EWQHFGFKES | |
| | PTSSYQDLSD FYREVEPQGY QVKFVDINAD YINELVEQGQ LYLFQIYNKD | |
| | FSPKAHGKPN LHTLYFKALF SKDNLANPIY KLNGEAQIFY RKASLDMNET | |
| | TIHRAGEVLE NKNPDNPKKR QFVYDIIKDK RYTQDKEMLH VPITMNFGVQ | |
| | GMTIKEFNKK VNQSIQQYDE VNVIGIDRGE RHLLYLTVIN SKGEILEQRS | |
| | LNDITTASAN GTQMTTPYHK ILDKREIERL NARVGWGEIE TIKELKSGYL | |
| | SHVVHQISQL MLKYNAIVVL EDLNFGEKRG REKVEKQIYQ NFENALIKKL | |
| | NHLVLKDEAD DEIGSYKNAL QLTNNFTDLK SIGKQTGELF YVPAWNTSKI | |
| | DPETGFVDLL KPRYENIAQS QAFFGKEDKI CYNADKDYFE FHIDYAKFTD | |
| | KAKNSRQIWK ICSHGDKRYV YDKTANQNKG ATKGINVNDE LKSLFARHHI | |
| | NDKQPNLVMD ICQNNDKEFH KSLIYLLKTL LALRYSNASS DEDFILSPVA | |
| | NDEGMFENSA LADDTQPQNA DANGAYHIAL KGLWVLEQIK NSDDLNKVKL | |
| | AIDNQTWLNF AQNR | |
| WP_020988726.1 | MEDYSGFVNI YSIQKTLRFE LKPVGKTLEH IEKKGFLKKD KIRAEDYKAV | (SEQ |
| type V CRISPR- | KKIIDKYHRA YIEEVEDSVL HQKKKKDKTR FSTQFIKEIK EFSELYYKTE | ID |
| associated | KNIPDKERLE ALSEKLRKML VGAFKGEFSE EVAEKYKNLF SKELIRNEIE | NO: |
| protein Cpf1 | KFCETDEERK QVSNFKSFTT YFTGFHSNRQ NIYSDEKKST AIGYRIIHQN | 137) |
| [Leptospira | LPKFLDNLKI IESIQRRFKD FPWSDLKKNL KKIDKNIKLT EYFSIDGFVN | |
| inadai] | VLNQKGIDAY NTILGGKSEE SGEKIQGLNE YINLYRQKNN IDRKNLPNVK | |
| | ILFKQILGDR ETKSFIPEAF PDDQSVLNSI TEFAKYLKLD KKKKSIIAEL | |
| | KKFLSSENRY ELDGIYLAND NSLASISTEL FDDWSFIKKS VSFKYDESVG | |
| | DPKKKIKSPL KYEKEKEKWL KQKYYTISFL NDAIESYSKS QDEKRVKIRL | |
| | EAYFAEFKSK DDAKKQFDLL ERIEEAYAIV EPLLGAEYPR DRNLKADKKE | |
| | VGKIKDELDS IKSLQFFLKP LLSAEIFDEK DLGFYNQLEG YYEEIDSIGH | |
| | LYNKVRNYLT GKIYSKEKFK LNFENSTLLK GWDENREVAN LCVIFREDQK | |
| | YYLGVMDKEN NTILSDIPKV KPNELFYEKM VYKLIPTPHM QLPRIIFSSD | |
| | NLSIYNPSKS ILKIREAKSF KEGKNFKLKD CHKFIDFYKE SISKNEDWSR | |
| | FDFKESKTSS YENISEFYRE VERQGYNLDF KKVSKFYIDS LVEDGKLYLE | |
| | QIYNKDESIF SKGKPNLHTI YFRSLESKEN LKDVCLKLNG EAEMFFRKKS | |
| | INYDEKKKRE GHHPELFEKL KYPILKDKRY SEDKFQFHLP ISLNFKSKER | |
| | LNENLKVNEF LKRNKDINII GIDRGERNLL YLVMINQKGE ILKQTLLDSM | |
| | QSGKGRPEIN YKEKLQEKEI ERDKARKSWG TVENIKELKE GYLSIVIHQI | |
| | SKLMVENNAI VVLEDLNIGF KRGRQKVERQ VYQKFEKMLI DKLNFLVEKE | |
| | NKPTEPGGVL KAYQLTDEFQ SFEKLSKQTG FLFYVPSWNT SKIDPRTGFI | |
| | DFLHPAYENI EKAKQWINKF DSIRENSKMD WFEFTADTRK FSENLMLGKN | |
| | RVWVICTTNV ERYFTSKTAN SSIQYNSIQI TEKLKELFVD IPFSNGQDLK | |
| | PEILRKNDAV FFKSLLFYIK TTLSLRQNNG KKGEEEKDFI LSPVVDSKGR | |
| | FFNSLEASDD EPKDADANGA YHIALKGLMN LLVLNETKEE NLSRPKWKIK | |
| | NKDWLEFVWE RNR | |
| WP_023936172.1 | MPWIDLKDFT NLYPVSKTLR FELKPVGKTL ENIEKAGILK EDEHRAESYR | (SEQ |
| type V CRISPR- | RVKKIIDTYH KVFIDSSLEN MAKMGIENEI KAMLQSFCEL YKKDHRTEGE | ID |
| associated | DKALDKIRAV LRGLIVGAFT GVCGRRENTV QNEKYESLFK EKLIKEILPD | NO: |
| protein Cpf1 | FVLSTEAESL PFSVEEATRS LKEFDSFTSY FAGFYENRKN IYSTKPQSTA | 138) |
| [Porphyromonas | IAYRLIHENL PKFIDNILVF QKIKEPIAKE LEHIRADESA GGYIKKDERL | |
| crevioricanis] | EDIFSLNYYI HVLSQAGIEK YNALIGKIVT EGDGEMKGLN EHINLYNQQR | |
| | GREDRLPLER PLYKQILSDR EQLSYLPESF EKDEELLRAL KEFYDHIAED | |
| | ILGRTQQLMT SISEYDLSRI YVRNDSQLTD ISKKMLGDWN AIYMARERAY | |
| | DHEQAPKRIT AKYERDRIKA LKGEESISLA NLNSCIAFLD NVRDCRVDTY | |
| | LSTLGQKEGP HGLSNLVENV FASYHEAEQL LSFPYPEENN LIQDKDNVVL | |
| | IKNLLDNISD LQRFLKPLWG MGDEPDKDER FYGEYNYIRG ALDQVIPLYN | |
| | KVRNYLTRKP YSTRKVKLNF GNSQLLSGWD RNKEKDNSCV ILRKGQNFYL | |
| | AIMNNRHKRS FENKVLPEYK EGEPYFEKMD YKFLPDPNKM LPKVELSKKG | |
| | IEIYEPSPKL LEQYGHGTHK KGDTESMDDL HELIDFFKHS IEAHEDWKQF | |
| | GFKFSDTATY ENVSSFYREV EDQGYKLSFR KVSESYVYSL IDQGKLYLFQ | |
| | IYNKDFSPCS KGTPNLHTLY WRMLEDERNL ADVIYKLDGK AEIFFREKSL | |
| | KNDHPTHPAG KPIKKKSRQK KGEESLFEYD LVKDRRYTMD KFQFHVPITM | |
| | NFKCSAGSKV NDMVNAHIRE AKDMHVIGID RGERNLLYIC VIDSRGTILD | |
| | QISLNTINDI DYHDLLESRD KDRQQERRNW QTIEGIKELK QGYLSQAVHR | |
| | IAELMVAYKA VVALEDLNMG FKRGRQKVES SVYQQFEKQL IDKLNYLVDK | |
| | KKRPEDIGGL LRAYQFTAPF KSFKEMGKQN GELFYIPAWN TSNIDPTTGE | |
| | VNLFHAQYEN VDKAKSFFQK FDSISYNPKK DWFEFAFDYK NFTKKAEGSR | |
| | SMWILCTHGS RIKNERNSQK NGQWDSEEFA LTEAFKSLFV RYEIDYTADL | |
| | KTAIVDEKQK DFFVDLLKLF KLTVQMRNSW KEKDLDYLIS PVAGADGRFF | |
| | DTREGNKSLP KDADANGAYN IALKGLWALR QIRQTSEGGK LKLAISNKEW | |
| | LQFVQERSYE KD | |
| WP_009217842.1 | MRKFNEFVGL YPISKTLRFE LKPIGKTLEH IQRNKLLEHD AVRADDYVKV | (SEQ |
| type V CRISPR- | KKIIDKYHKC LIDEALSGFT FDTEADGRSN NSLSEYYLYY NLKKRNEQEQ | ID |
| associated | KTFKTIQNNL RKQIVNKLTQ SEKYKRIDKK ELITTDLPDF LTNESEKELV | NO: |
| protein Cpf1 | EKFKNFTTYF TEFHKNRKNM YSKEEKSTAI AFRLINENLP KFVDNIAAFE | 139) |
| [Bacteroidetes | KVVSSPLAEK INALYEDEKE YLNVEEISRV FRLDYYDELL TQKQIDLYNA | |
| oral taxon 274] | IVGGRTEEDN KIQIKGLNQY INEYNQQQTD RSNRLPKLKP LYKQILSDRE | |
| | SVSWLPPKED SDKNLLIKIK ECYDALSEKE KVEDKLESIL KSLSTYDLSK | |
| | IYISNDSQLS YISQKMFGRW DIISKAIRED CAKRNPQKSR ESLEKFAERI | |
| | DKKLKTIDSI SIGDVDECLA QLGETYVKRV EDYFVAMGES EIDDEQTDTT | |
| | SFKKNIEGAY ESVKELLNNA DNITDNNLMQ DKGNVEKIKT LLDAIKDLQR | |
| | FIKPLLGKGD EADKDGVFYG EFTSLWTKLD QVTPLYNMVR NYLTSKPYST | |
| | KKIKLNFENS TLMDGWDLNK EPDNTTVIFC KDGLYYLGIM GKKYNRVFVD | |
| | REDLPHDGEC YDKMEYKLLP GANKMLPKVF FSETGIQRFL PSEELLGKYE | |
| | RGTHKKGAGF DLGDCRALID FFKKSIERHD DWKKEDEKES DTSTYQDISE | |
| | FYREVEQQGY KMSFRKVSVD YIKSLVEEGK LYLFQIYNKD FSAHSKGTPN | |
| | MHTLYWKMLF DEENLKDVVY KLNGEAEVFF RKSSITVQSP THPANSPIKN | |
| | KNKDNQKKES KFEYDLIKDR RYTVDKFLFH VPITMNFKSV GGSNINQLVK | |
| | RHIRSATDLH IIGIDRGERH LLYLTVIDSR GNIKEQFSLN EIVNEYNGNT | |
| | YRTDYHELLD TREGERTEAR RNWQTIQNIR ELKEGYLSQV IHKISELAIK | |
| | YNAVIVLEDL NFGFMRSRQK VEKQVYQKFE KMLIDKLNYL VDKKKPVAET | |
| | GGLLRAYQLT GEFESFKTLG KQSGILFYVP AWNTSKIDPV TGFVNLEDTH | |
| | YENIEKAKVE FDKEKSIRYN SDKDWFEFVV DDYTRFSPKA EGTRRDWTIC | |
| | TQGKRIQICR NHQRNNEWEG QEIDLTKAFK EHFEAYGVDI SKDLREQINT | |
| | QNKKEFFEEL LRLLRLTLQM RNSMPSSDID YLISPVANDT GCFFDSRKQA | |
| | ELKENAVLPM NADANGAYNI ARKGLLAIRK MKQEENDSAK ISLAISNKEW | |
| | LKFAQTKPYL ED | |
| WP_036890108.1 | MDSLKDFTNL YPVSKTLRFE LKPVGKTLEN IEKAGILKED EHRAESYRRV | (SEQ |
| type V CRISPR- | KKIIDTYHKV FIDSSLENMA KMGIENEIKA MLQSFCELYK KDHRTEGEDK | ID |
| associated | ALDKIRAVLR GLIVGAFTGV CGRRENTVQN EKYESLFKEK LIKEILPDFV | NO: |
| protein Cpf1 | LSTEAESLPF SVEEATRSLK EFDSFTSYFA GFYENRKNIY STKPQSTAIA | 140) |
| [Porphyromonas | YRLIHENLPK FIDNILVFQK IKEPIAKELE HIRADESAGG YIKKDERLED | |
| crevioricanis] | IFSLNYYIHV LSQAGIEKYN ALIGKIVTEG DGEMKGLNEH INLYNQQRGR | |
| | EDRLPLERPL YKQILSDREQ LSYLPESFEK DEELLRALKE FYDHIAEDIL | |
| | GRTQQLMTSI SEYDLSRIYV RNDSQLTDIS KKMLGDWNAI YMARERAYDH | |
| | EQAPKRITAK YERDRIKALK GEESISLANL NSCIAFLDNV RDCRVDTYLS | |
| | TLGQKEGPHG LSNLVENVFA SYHEAEQLLS FPYPEENNLI QDKDNVVLIK | |
| | NLLDNISDLQ RFLKPLWGMG DEPDKDERFY GEYNYIRGAL DQVIPLYNKV | |
| | RNYLTRKPYS TRKVKLNFGN SQLLSGWDRN KEKDNSCVIL RKGQNFYLAI | |
| | MNNRHKRSFE NKMLPEYKEG EPYFEKMDYK FLPDPNKMLP KVFLSKKGIE | |
| | IYKPSPKLLE QYGHGTHKKG DTFSMDDLHE LIDFFKHSIE AHEDWKQFGF | |
| | KFSDTATYEN VSSFYREVED QGYKLSFRKV SESYVYSLID QGKLYLFQIY | |
| | NKDFSPCSKG TPNLHTLYWR MLEDERNLAD VIYKLDGKAE IFFREKSLKN | |
| | DHPTHPAGKP IKKKSRQKKG EESLFEYDLV KDRRYTMDKF QFHVPITMNF | |
| | KCSAGSKVND MVNAHIREAK DMHVIGIDRG ERNLLYICVI DSRGTILDQI | |
| | SLNTINDIDY HDLLESRDKD RQQEHRNWQT IEGIKELKQG YLSQAVHRIA | |
| | ELMVAYKAVV ALEDLNMGFK RGRQKVESSV YQQFEKQLID KLNYLVDKKK | |
| | RPEDIGGLLR AYQFTAPFKS FKEMGKQNGE LEYIPAWNTS NIDPTTGFVN | |
| | LFHVQYENVD KAKSFFQKED SISYNPKKDW FEFAFDYKNF TKKAEGSRSM | |
| | WILCTHGSRI KNERNSQKNG QWDSEEFALT EAFKSLFVRY EIDYTADLKT | |
| | AIVDEKQKDF FVDLLKLFKL TVQMRNSWKE KDLDYLISPV AGADGRFEDT | |
| | REGNKSLPKD ADANGAYNIA LKGLWALRQI RQTSEGGKLK LAISNKEWLQ | |
| | FVQERSYEKD | |
| WP_036887416.1 | MDSLKDFTNL YPVSKTLRFE LKPVGKTLEN IEKAGILKED EHRAESYRRV | (SEQ |
| type V CRISPR- | KKIIDTYHKV FIDSSLENMA KMGIENEIKA MLQSFCELYK KDHRTEGEDK | ID |
| associated | ALDKIRAVLR GLIVGAFTGV CGRRENTVQN EKYESLFKEK LIKEILPDFV | NO: |
| protein Cpf1 | LSTEAESLPF SVEEATRSLK EFDSFTSYFA GFYENRKNIY STKPQSTAIA | 141) |
| [Porphyromonas | YRLIHENLPK FIDNILVFQK IKEPIAKELE HIRADESAGG YIKKDERLED | |
| crevioricanis] | IFSLNYYIHV LSQAGIEKYN ALIGKIVTEG DGEMKGLNEH INLYNQQRGR | |
| | EDRLPLERPL YKQILSDREQ LSYLPESFEK DEELLRALKE FYDHIAEDIL | |
| | GRTQQLMTSI SEYDLSRIYV RNDSQLTDIS KKMLGDWNAI YMARERAYDH | |
| | EQAPKRITAK YERDRIKALK GEESISLANL NSCIAFLDNV RDCRVDTYLS | |
| | TLGQKEGPHG LSNLVENVFA SYHEAEQLLS FPYPEENNLI QDKDNVVLIK | |
| | NLLDNISDLQ RFLKPLWGMG DEPDKDERFY GEYNYIRGAL DQVIPLYNKV | |
| | RNYLTRKPYS TRKVKLNFGN SQLLSGWDRN KEKDNSCVIL RKGQNFYLAI | |
| | MNNRHKRSFE NKVLPEYKEG EPYFEKMDYK FLPDPNKMLP KVELSKKGIE | |
| | IYKPSPKLLE QYGHGTHKKG DTFSMDDLHE LIDFFKHSIE AHEDWKQFGE | |
| | KFSDTATYEN VSSFYREVED QGYKLSFRKV SESYVYSLID QGKLYLFQIY | |
| | NKDESPCSKG TPNLHTLYWR MLEDERNLAD VIYKLDGKAE IFFREKSLKN | |
| | DHPTHPAGKP IKKKSRQKKG EESLFEYDLV KDRHYTMDKF QFHVPITMNE | |
| | KCSAGSKVND MVNAHIREAK DMHVIGIDRG ERNLLYICVI DSRGTILDQI | |
| | SLNTINDIDY HDLLESRDKD RQQERRNWQT IEGIKELKQG YLSQAVHRIA | |
| | ELMVAYKAVV ALEDLNMGFK RGRQKVESSV YQQFEKQLID KLNYLVDKKK | |
| | RPEDIGGLLR AYQFTAPFKS FKEMGKQNGF LFYIPAWNTS NIDPTTGFVN | |
| | LFHAQYENVD KAKSFFQKED SISYNPKKDW FEFAFDYKNF TKKAEGSRSM | |
| | WILCTHGSRI KNFRNSQKNG QWDSEEFALT EAFKSLFVRY EIDYTADLKT | |
| | AIVDEKQKDF FVDLLKLFKL TVQMRNSWKE KDLDYLISPV AGADGRFEDT | |
| | REGNKSLPKD ADANGAYNIA LKGLWALRQI RQTSEGGKLK LAISNKEWLQ | |
| | FVQERSYEKD | |
| WP_023941260.1 | MDSLKDFTNL YPVSKTLRFE LKPVGKTLEN IEKAGILKED EHRAESYRRV | (SEQ |
| type V CRISPR- | KKIIDTYHKV FIDSSLENMA KMGIENEIKA MLQSFCELYK KDHRTEGEDK | ID |
| associated | ALDKIRAVLR GLIVGAFTGV CGRRENTVQN EKYESLFKEK LIKEILPDFV | NO: |
| protein Cpf1 | LSTEAESLPF SVEEATRSLK EFDSFTSYFA GFYENRKNIY STKPQSTAIA | 142) |
| [Porphyromonas | YRLIHENLPK FIDNILVFQK IKEPIAKELE HIRADFSAGG YIKKDERLED | |
| crevioricanis] | IFSLNYYIHV LSQAGIEKYN ALIGKIVTEG DGEMKGLNEH INLYNQQRGR | |
| | EDRLPLERPL YKQILSDREQ LSYLPESFEK DEELLRALKE FYDHIAEDIL | |
| | GRTQQLMTSI SEYDLSRIYV RNDSQLTDIS KKMLGDWNAI YMARERAYDH | |
| | EQAPKRITAK YERDRIKALK GEESISLANL NSCIAFLDNV RDCRVDTYLS | |
| | TLGQKEGPHG LSNLVENVFA SYHEAEQLLS FPYPEENNLI QDKDNVVLIK | |
| | NLLDNISDLQ RFLKPLWGMG DEPDKDERFY GEYNYIRGAL DQVIPLYNKV | |
| | RNYLTRKPYS TRKVKLNFGN SQLLSGWDRN KEKDNSCVIL RKGQNFYLAI | |
| | MNNRHKRSFE NKVLPEYKEG EPYFEKMDYK FLPDPNKMLP KVELSKKGIE | |
| | IYKPSPKLLE QYGHGTHKKG DTFSMDDLHE LIDFFKHSIE AHEDWKQFGF | |
| | KESDTATYEN VSSFYREVED QGYKLSFRKV SESYVYSLID QGKLYLFQIY | |
| | NKDFSPCSKG TPNLHTLYWR MLEDERNLAD VIYKLDGKAE IFFREKSLKN | |
| | DHPTHPAGKP IKKKSRQKKG EESLFEYDLV KDRRYTMDKF QFHVPITMNE | |
| | KCSAGSKVND MVNAHIREAK DMHVIGIDRG ERNLLYICVI DSRGTILDQI | |
| | SLNTINDIDY HDLLESRDKD RQQERRNWQT IEGIKELKQG YLSQAVHRIA | |
| | ELMVAYKAVV ALEDLNMGFK RGRQKVESSV YQQFEKQLID KLNYLVDKKK | |
| | RPEDIGGLLR AYQFTAPFKS FKEMGKQNGE LFYIPAWNTS NIDPTTGEVN | |
| | LFHAQYENVD KAKSFFQKED SISYNPKKDW FEFAFDYKNF TKKAEGSRSM | |
| | WILCTHGSRI KNFRNSQKNG QWDSEEFALT EAFKSLEVRY EIDYTADLKT | |
| | AIVDEKQKDF FVDLLKLFKL TVQMRNSWKE KDLDYLISPV AGADGRFEDT | |
| | REGNKSLPKD ADANGAYNIA LKGLWALRQI RQTSEGGKLK LAISNKEWLQ | |
| | FVQERSYEKD | |
| WP_037975888.1 | MANSLKDFTN IYQLSKTLRF ELKPIGKTEE HINRKLIIMH DEKRGEDYKS | (SEQ |
| type V CRISPR- | VTKLIDDYHR KFIHETLDPA HEDWNPLAEA LIQSGSKNNK ALPAEQKEMR | ID |
| associated | EKIISMFTSQ AVYKKLEKKE LFSELLPEMI KSELVSDLEK QAQLDAVKSF | NO: |
| protein Cpf1 | DKFSTYFTGF HENRKNIYSK KDTSTSIAFR IVHQNFPKEL ANVRAYTLIK | 143) |
| [Synergistes | ERAPEVIDKA QKELSGILGG KTLDDIFSIE SENNVLTQDK IDYYNQIIGG | |
| jonesii] | VSGKAGDKKL RGVNEFSNLY RQQHPEVASL RIKMVPLYKQ ILSDRTTLSF | |
| | VPEALKDDEQ AINAVDGLRS ELERNDIENR IKRLFGKNNL YSLDKIWIKN | |
| | SSISAFSNEL FKNWSFIEDA LKEFKENEEN GARSAGKKAE KWLKSKYFSF | |
| | ADIDAAVKSY SEQVSADISS APSASYFAKE TNLIETAAEN GRKFSYFAAE | |
| | SKAFRGDDGK TEIIKAYLDS LNDILHCLKP FETEDISDID TEFYSAFAEI | |
| | YDSVKDVIPV YNAVRNYTTQ KPFSTEKFKL NFENPALAKG WDKNKEQNNT | |
| | AIILMKDGKY YLGVIDKNNK LRADDLADDG SAYGYMKMNY KFIPTPHMEL | |
| | PKVFLPKRAP KRYNPSREIL LIKENKTFIK DKNFNRTDCH KLIDFFKDSI | |
| | NKHKDWRTFG FDESDTDSYE DISDFYMEVQ DQGYKLTFTR LSAEKIDKWV | |
| | EEGRLFLFQI YNKDFADGAQ GSPNLHTLYW KAIFSEENLK DVVLKLNGEA | |
| | ELFFRRKSID KPAVHAKGSM KVNRRDIDGN PIDEGTYVEI CGYANGKRDM | |
| | ASLNAGARGL IESGLVRITE VKHELVKDKR YTIDKYFFHV PFTINFKAQG | |
| | QGNINSDVNL FLRNNKDVNI IGIDRGERNL VYVSLIDRDG HIKLQKDENI | |
| | IGGMDYHAKL NQKEKERDTA RKSWKTIGTI KELKEGYLSQ VVHEIVRLAV | |
| | DNNAVIVMED LNIGFKRGRF KVEKQVYQKF EKMLIDKLNY LVFKDAGYDA | |
| | PCGILKGLQL TEKFESFTKL GKQCGIIFYI PAGYTSKIDP TTGFVNLENI | |
| | NDVSSKEKQK DFIGKLDSIR FDAKRDMFTF EFDYDKERTY QTSYRKKWAV | |
| | WTNGKRIVRE KDKDGKFRMN DRLLTEDMKN ILNKYALAYK AGEDILPDVI | |
| | SRDKSLASEI FYVEKNTLQM RNSKRDTGED FIISPVLNAK GRFFDSRKTD | |
| | AALPIDADAN GAYHIALKGS LVLDAIDEKL KEDGRIDYKD MAVSNPKWFE | |
| | FMQTRKFDF | |
| WP_081839471.1 | MENMANSLKD FTNIYQLSKT LRFELKPIGK TEEHINRKLI IMHDEKRGED | (SEQ |
| type V CRISPR- | YKSVTKLIDD YHRKFIHETL DPAHEDWNPL AEALIQSGSK NNKALPAEQK | ID |
| associated | EMREKIISME TSQAVYKKLF KKELFSELLP EMIKSELVSD LEKQAQLDAV | NO: |
| protein Cpf1 | KSFDKFSTYF TGFHENRKNI YSKKDTSTSI AFRIVHQNEP KFLANVRAYT | 144) |
| [Synergistes | LIKERAPEVI DKAQKELSGI LGGKTLDDIF SIESENNVLT QDKIDYYNQI | |
| jonesii] | IGGVSGKAGD KKLRGVNEFS NLYRQQHPEV ASLRIKMVPL YKQILSDRTT | |
| | LSFVPEALKD DEQAINAVDG LRSELERNDI FNRIKRLEGK NNLYSLDKIW | |
| | IKNSSISAFS NELFKNWSFI EDALKEFKEN EFNGARSAGK KAEKWLKSKY | |
| | FSFADIDAAV KSYSEQVSAD ISSAPSASYF AKFTNLIETA AENGRKESYF | |
| | AAESKAFRGD DGKTEIIKAY LDSLNDILHC LKPFETEDIS DIDTEFYSAF | |
| | AEIYDSVKDV IPVYNAVRNY TTQKPESTEK FKLNFENPAL AKGWDKNKEQ | |
| | NNTAIILMKD GKYYLGVIDK NNKLRADDLA DDGSAYGYMK MNYKFIPTPH | |
| | MELPKVELPK RAPKRYNPSR EILLIKENKT FIKDKNENRT DCHKLIDFFK | |
| | DSINKHKDWR TFGFDESDTD SYEDISDFYM EVQDQGYKLT FTRLSAEKID | |
| | GEAELFFRRK SIDKPAVHAK GAQGSPNLHT LYWKAIFSEE NLKDVVLKLN | |
| | KWVEEGRLFL FQIYNKDFAD GSMKVNRRDI DGNPIDEGTY VEICGYANGK | |
| | RDMASLNAGA RGLIESGLVR ITEVKHELVK DKRYTIDKYF FHVPFTINEK | |
| | AQGQGNINSD VNLFLRNNKD VNIIGIDRGE RNLVYVSLID RDGHIKLQKD | |
| | FNIIGGMDYH AKLNQKEKER DTARKSWKTI GTIKELKEGY LSQVVHEIVR | |
| | LAVQNNAVIV MEDLNIGFKR GRFKVEKQVY QKFEKMLIDK LNYLVFKDAG | |
| | YDAPCGILKG LQLTEKFESF TKLGKQCGII FYIPAGYTSK IDPTTGFVNL | |
| | FNINDVSSKE KQKDFIGKLD SIRFDAKRDM FTFEFDYDKF RTYQTSYRKK | |
| | WAVWINGKRI VREKDKDGKF RMNDRLLTED MKNILNKYAL AYKAGEDILP | |
| | DVISRDKSLA SEIFYVFKNT LQMRNSKRDT GEDFIISPVL NAKGRFFDSR | |
| | KTDAALPIDA DANGAYHIAL KGSLVLDAID EKLKEDGRID YKDMAVSNPK | |
| | WFEFMQTRKF DF | |
| WP_006283774.1 | MQINNLKIIY MKFTDFTGLY SLSKTLRFEL KPIGKTLENI KKAGLLEQDQ | (SEQ |
| type V CRISPR- | HRADSYKKVK KIIDEYHKAF IEKSLSNFEL KYQSEDKLDS LEEYLMYYSM | ID |
| associated | KRIEKTEKDK FAKIQDNLRK QIADHLKGDE SYKTIFSKDL IRKNLPDFVK | NO: |
| protein Cpf1 | SDEERTLIKE FKDFTTYFKG FYENRENMYS AEDKSTAISH HLDYFSMVMT | 145) |
| [Prevotella | VDNINAFSKI ILIPELREKL NQIYQDFEEY LNVESIDEIF RIIHENLPKF | |
| bryantii B14] | QKQIEVYNAI IGGKSTNDKK IQGLNEYINL YNQKHKDCKL PKLKLLFKQI | |
| | LSDRIAISWL PDNFKDDQEA LDSIDTCYKN LLNDGNVLGE GNLKLLLENI | |
| | DTYNLKGIFI RNDLQLTDIS QKMYASWNVI QDAVILDLKK QVSRKKKESA | |
| | EDYNDRLKKL YTSQESFSIQ YLNDCLRAYG KTENIQDYFA KLGAVNNEHE | |
| | QTINLFAQVR NAYTSVQAIL TTPYPENANL AQDKETVALI KNLLDSLKRL | |
| | QRFIKPLLGK GDESDKDERF YGDFTPLWET LNQITPLYNM VRNYMTRKPY | |
| | SQEKIKLNFE NSTLLGGWDL NKEHDNTAII LRKNGLYYLA IMKKSANKIF | |
| | DKDKLDNSGD CYEKMVYKLL PGANKMLPKV FFSKSRIDEF KPSENIIENY | |
| | KKGTHKKGAN FNLADCHNLI DFFKSSISKH EDWSKENFHF SDTSSYEDLS | |
| | DFYREVEQQG YSISFCDVSV EYINKMVEKG DLYLFQIYNK DFSEFSKGTP | |
| | NMHTLYWNSL FSKENLNNII YKLNGQAEIF FRKKSLNYKR PTHPAHQAIK | |
| | NKNKCNEKKE SIFDYDLVKD KRYTVDKFQF HVPITMNFKS TGNTNINQQV | |
| | IDYLRTEDDT HIIGIDRGER HLLYLVVIDS HGKIVEQFTL NEIVNEYGGN | |
| | IYRTNYHDLL DTREQNREKA RESWQTIENI KELKEGYISQ VIHKITDLMQ | |
| | KYHAVVVLED LNMGFMRGRQ KVEKQVYQKF EEMLINKLNY LVNKKADQNS | |
| | AGGLLHAYQL TSKFESFQKL GKQSGELFYI PAWNTSKIDP VTGFVNLEDT | |
| | RYESIDKAKA FFGKEDSIRY NADKDWFEFA FDYNNFTTKA EGTRTNWTIC | |
| | TYGSRIRTFR NQAKNSQWDN EEIDLTKAYK AFFAKHGINI YDNIKEAIAM | |
| | ETEKSFFEDL LHLLKLTLQM RNSITGTTTD YLISPVHDSK GNFYDSRICD | |
| | NSLPANADAN GAYNIARKGL MLIQQIKDST SSNRFKFSPI TNKDWLIFAQ | |
| | EKPYLND | |
| WP_024988992 | MNIKNFTGLY PLSKTLRFEL KPIGKTKENI EKNGILTKDE QRAKDYLIVK | (SEQ |
| type V CRISPR- | GFIDEYHKQF IKDRLWDEKL PLESEGEKNS LEEYQELYEL TKRNDAQEAD | ID |
| associated | FTEIKDNLRS SITEQLTKSG SAYDRIFKKE FIREDLVNEL EDEKDKNIVK | NO: |
| protein Cpf1 | QFEDFTTYFT GFYENRKNMY SSEEKSTAIA YRLIHQNLPK FMDNMRSFAK | 146) |
| [Prevotella | IANSSVSEHF SDIYESWKEY LNVNSIEEIF QLDYFSETLT QPHIEVYNYI | |
| albensis] | IGKKVLEDGT EIKGINEYVN LYNQQQKDKS KRLPFLVPLY KQILSDREKL | |
| | SWIAEEFDSD KKMLSAITES YNHLHNVLMG NENESLRNLL LNIKDYNLEK | |
| | INITNDLSLT EISQNLFGRY DVFTNGIKNK LRVLTPRKKK ETDENFEDRI | |
| | NKIFKTQKSF SIAFLNKLPQ PEMEDGKPRN IEDYFITQGA INTKSIQKED | |
| | IFAQIENAYE DAQVFLQIKD TDNKLSQNKT AVEKIKTLLD ALKELQHFIK | |
| | PLLGSGEENE KDELFYGSFL AIWDELDTIT PLYNKVRNWL TRKPYSTEKI | |
| | KLNFDNAQLL GGWDVNKEHD CAGILLRKND SYYLGIINKK TNHIFDTDIT | |
| | PSDGECYDKI DYKLLPGANK MLPKVFFSKS RIKEFEPSEA IINCYKKGTH | |
| | KKGKNFNLTD CHRLINFEKT SIEKHEDWSK FGFKFSDTET YEDISGFYRE | |
| | VEQQGYRLTS HPVSASYIHS LVKEGKLYLF QIWNKDESQF SKGTPNLHTL | |
| | YWKMLFDKRN LSDVVYKLNG QAEVFYRKSS IEHQNRIIHP AQHPITNKNE | |
| | LNKKHTSTFK YDIIKDRRYT VDKFQFHVPI TINFKATGQN NINPIVQEVI | |
| | RQNGITHIIG IDRGERHLLY LSLIDLKGNI IKQMTLNEII NEYKGVTYKT | |
| | NYHNLLEKRE KERTEARHSW SSIESIKELK DGYMSQVIHK ITDMMVKYNA | |
| | IVVLEDLNGG FMRGRQKVEK QVYQKFEKKL IDKLNYLVDK KLDANEVGGV | |
| | LNAYQLTNKF ESFKKIGKQS GELFYIPAWN TSKIDPITGF VNLENTRYES | |
| | IKETKVFWSK FDIIRYNKEK NWFEFVEDYN TFTTKAEGTR TKWTLCTHGT | |
| | RIQTERNPEK NAQWDNKEIN LTESFKALFE KYKIDITSNL KESIMQETEK | |
| | KFFQELHNLL HLTLQMRNSV TGTDIDYLIS PVADEDGNFY DSRINGKNEP | |
| | ENADANGAYN IARKGLMLIR QIKQADPQKK FKFETITNKD WLKFAQDKPY | |
| | LKD | |
| WP_039658684.1 | MQTLFENFTN QYPVSKTLRF ELIPQGKTKD FIEQKGLLKK DEDRAEKYKK | (SEQ |
| type V CRISPR- | VKNIIDEYHK DFIEKSLNGL KLDGLEKYKT LYLKQEKDDK DKKAFDKEKE | ID |
| associated | NLRKQIANAF RNNEKFKTLF AKELIKNDLM SFACEEDKKN VKEFEAFTTY | NO: |
| protein Cpf1 | FTGFHQNRAN MYVADEKRTA IASRLIHENL PKFIDNIKIF EKMKKEAPEL | 147) |
| [Smithella sp. | LSPFNQTLKD MKDVIKGTTL EEIFSLDYEN KTLTQSGIDI YNSVIGGRTP | |
| SC_K08D17] | EEGKTKIKGL NEYINTDENQ KQTDKKKRQP KFKQLYKQIL SDRQSLSFIA | |
| | EAFKNDTEIL EAIEKFYVNE LLHFSNEGKS TNVLDAIKNA VSNLESENLT | |
| | KMYFRSGASL TDVSRKVFGE WSIINRALDN YYATTYPIKP REKSEKYEER | |
| | KEKWLKQDEN VSLIQTAIDE YDNETVKGKN SGKVIADYFA KFCDDKETDL | |
| | IQKVNEGYIA VKDLLNTPCP ENEKLGSNKD QVKQIKAFMD SIMDIMHFVR | |
| | PLSLKDTDKE KDETFYSLFT PLYDHLTQTI ALYNKVRNYL TQKPYSTEKI | |
| | KLNFENSTLL GGWDLNKETD NTAIILRKDN LYYLGIMDKR HNRIFRNVPK | |
| | ADKKDFCYEK MVYKLLPGAN KMLPKVFFSQ SRIQEFTPSA KLLENYANET | |
| | HKKGDNFNLN HCHKLIDFFK DSINKHEDWK NEDFRESATS TYADLSGFYH | |
| | EVEHQGYKIS FQSVADSFID DLVNEGKLYL FQIYNKDESP FSKGKPNLHT | |
| | LYWKMLEDEN NLKDVVYKLN GEAEVFYRKK SIAEKNTTIH KANESIINKN | |
| | PDNPKATSTF NYDIVKDKRY TIDKFQFHIP ITMNFKAEGI FNMNQRVNQF | |
| | LKANPDINII GIDRGERHLL YYALINQKGK ILKQDTLNVI ANEKQKVDYH | |
| | NLLDKKEGDR ATARQEWGVI ETIKELKEGY LSQVIHKLTD LMIENNAIIV | |
| | MEDLNFGEKR GRQKVEKQVY QKFEKMLIDK LNYLVDKNKK ANELGGLLNA | |
| | FQLANKFESF QKMGKQNGFI FYVPAWNTSK TDPATGFIDF LKPRYENLNQ | |
| | AKDFFEKEDS IRLNSKADYF EFAFDEKNFT EKADGGRTKW TVCTTNEDRY | |
| | AWNRALNNNR GSQEKYDITA ELKSLEDGKV DYKSGKDLKQ QIASQESADE | |
| | FKALMKNLSI TLSLRHNNGE KGDNEQDYIL SPVADSKGRF FDSRKADDDM | |
| | PKNADANGAY HIALKGLWCL EQISKTDDLK KVKLAISNKE WLEFVQTLKG | |
| WP_037385181 | MQTLFENFTN QYPVSKTLRF ELIPQGKTKD FIEQKGLLKK DEDRAEKYKK | (SEQ |
| type V CRISPR- | VKNIIDEYHK DFIEKSLNGL KLDGLEEYKT LYLKQEKDDK DKKAFDKEKE | ID |
| associated | NLRKQIANAF RNNEKFKTLF AKELIKNDLM SFACEEDKKN VKEFEAFTTY | NO: |
| protein Cpf1 | FTGFHQNRAN MYVADEKRTA IASRLIHENL PKFIDNIKIF EKMKKEAPEL | 148) |
| [Smithella sp. | LSPFNQTLKD MKDVIKGTTL EEIFSLDYEN KTLTQSGIDI YNSVIGGRTP | |
| SCADC] | EEGKTKIKGL NEYINTDENQ KQTDKKKRQP KFKQLYKQIL SDRQSLSFIA | |
| | EAFKNDTEIL EAIEKFYVNE LLHFSNEGKS TNVLDAIKNA VSNLESENLT | |
| | KIYFRSGTSL TDVSRKVFGE WSIINRALDN YYATTYPIKP REKSEKYEER | |
| | KEKWLKQDEN VSLIQTAIDE YDNETVKGKN SGKVIVDYFA KFCDDKETDL | |
| | IQKVNEGYIA VKDLLNTPYP ENEKLGSNKD QVKQIKAFMD SIMDIMHFVR | |
| | PLSLKDTDKE KDETFYSLFT PLYDHLTQTI ALYNKVRNYL TQKPYSTEKI | |
| | KLNFENSTLL GGWDLNKETD NTAIILRKEN LYYLGIMDKR HNRIFRNVPK | |
| | ADKKDSCYEK MVYKLLPGAN KMLPKVFFSQ SRIQEFTPSA KLLENYENET | |
| | HKKGDNENLN HCHQLIDFFK DSINKHEDWK NEDFRESATS TYADLSGFYH | |
| | EVEHQGYKIS FQSIADSFID DLVNEGKLYL FQIYNKDESP FSKGKPNLHT | |
| | LYWKMLEDEN NLKDVVYKLN GEAEVFYRKK SIAEKNTTIH KANESIINKN | |
| | PDNPKATSTF NYDIVKDKRY TIDKFQFHVP ITMNEKAEGI FNMNQRVNQF | |
| | LKANPDINII GIDRGERHLL YYTLINQKGK ILKQDTLNVI ANEKQKVDYH | |
| | NLLDKKEGDR ATARQEWGVI ETIKELKEGY LSQVIHKLTD LMIENNAIIV | |
| | MEDLNFGFKR GRQKVEKQVY QKFEKMLIDK LNYLVDKNKK ANELGGLLNA | |
| | FQLANKFESE QKMGKQNGFI FYVPAWNTSK TDPATGFIDF LKPRYENLKQ | |
| | AKDFFEKFDS IRLNSKADYF EFAFDEKNFT GKADGGRTKW TVCTTNEDRY | |
| | AWNRALNNNR GSQEKYDITA ELKSLEDGKV DYKSGKDLKQ QIASQELADE | |
| | FRTLMKYLSV TLSLRHNNGE KGETEQDYIL SPVADSMGKF FDSRKAGDDM | |
| | PKNADANGAY HIALKGLWCL EQISKTDDLK KVKLAISNKE WLEFMQTLKG | |
| WP_039871282.1 | MKFTDETGLY SLSKTLRFEL KPIGKTLENI KKAGLLEQDQ HRADSYKKVK | (SEQ |
| type V | KIIDEYHKAF IEKSLSNFEL KYQSEDKLDS LEEYLMYYSM KRIEKTEKDK | ID |
| CRISPR- | FAKIQDNLRK QIADHLKGDE SYKTIFSKDL IRKNLPDFVK SDEERTLIKE | NO: |
| associated | FKDETTYFKG FYENRENMYS AEDKSTAISH RIIHENLPKF VDNINAFSKI | 149) |
| protein Cpf1 | ILIPELREKL NQIYQDFEEY LNVESIDEIF HLDYFSMVMT QKQIEVYNAI | |
| [Prevotella | IGGKSTNDKK IQGLNEYINL YNQKHKDCKL PKLKLLFKQI LSDRIAISWL | |
| bryantii B14] | PDNEKDDQEA LDSIDTCYKN LLNDGNVLGE GNLKLLLENI DTYNLKGIFI | |
| | RNDLQLTDIS QKMYASWNVI QDAVILDLKK QVSRKKKESA EDYNDRLKKL | |
| | YTSQESFSIQ YLNDCLRAYG KTENIQDYFA KLGAVNNEHE QTINLFAQVR | |
| | NAYTSVQAIL TTPYPENANL AQDKETVALI KNLLDSLKRL QRFIKPLLGK | |
| | GDESDKDERF YGDFTPLWET LNQITPLYNM VRNYMTRKPY SQEKIKLNFE | |
| | NSTLLGGWDL NKEHDNTAII LRKNGLYYLA IMKKSANKIF DKDKLDNSGD | |
| | CYEKMVYKLL PGANKMLPKV FFSKSRIDEF KPSENIIENY KKGTHKKGAN | |
| | FNLADCHNLI DFFKSSISKH EDWSKENFHF SDTSSYEDLS DFYREVEQQG | |
| | YSISFCDVSV EYINKMVEKG DLYLFQIYNK DFSEFSKGTP NMHTLYWNSL | |
| | FSKENLNNII YKLNGQAEIF FRKKSLNYKR PTHPAHQAIK NKNKCNEKKE | |
| | SIFDYDLVKD KRYTVDKFQF HVPITMNEKS TGNTNINQQV IDYLRTEDDT | |
| | HIIGIDRGER HLLYLVVIDS HGKIVEQFTL NEIVNEYGGN IYRTNYHDLL | |
| | DTREQNREKA RESWQTIENI KELKEGYISQ VIHKITDLMQ KYHAVVVLED | |
| | LNMGFMRGRQ KVEKQVYQKF EEMLINKLNY LVNKKADQNS AGGLLHAYQL | |
| | TSKFESFQKL GKQSGFLFYI PAWNTSKIDP VTGFVNLEDT RYESIDKAKA | |
| | FFGKEDSIRY NADKDWFEFA FDYNNFTTKA EGTRTNWTIC TYGSRIRTER | |
| | NQAKNSQWDN EEIDLTKAYK AFFAKHGINI YDNIKEAIAM ETEKSFFEDL | |
| | LHLLKLTLQM RNSITGTTTD YLISPVHDSK GNFYDSRICD NSLPANADAN | |
| | GAYNIARKGL MLIQQIKDST SSNRFKFSPI TNKDWLIFAQ EKPYLND | |
| EKE28449.1 | MFKGDAFTGL YEVQKTLRFE LVPIGLTQSY LENDWVIQKD KEVEENYGKI | (SEQ |
| hypothetical | KAYFDLIHKE FVRQSLENAW LCQLDDFYEK YIELHNSLET RKDKNLAKQF | ID |
| protein | EKVMKSLKKE FVSFFDAKWN EWKQKFSFLK KWWIDVLNEK EVLDLMAEFY | NO: |
| ACD_3C00058G | PDEKELFDKF DKFFTYFSNF KESRKNFYAD DGRAWAIATR AIDENLITFI | 150) |
| 0015 [uncultured | KNIEDFKKLN SSFREFVNDN FSEEDKQIFE IDFYNNCLLQ PWIDKYNKIV | |
| bacterium (gcode | WWYSLENWEK VQWLNEKINN FKQNQNKSNS KDLKFPRMKL LYKQILGDKE | |
| 4)] | KKVYIDEIRD DKNLIDLIDN SKRRNQIKID NANDIINDFI NNNAKFELDK | |
| | IYLTRQSINT ISSKYFSSWD YIRWYFWTGE LQEFVSFYDL KETFWKIEYE | |
| | TLENIFKDCY VKGINTESQN NIVFETQGIY ENFLNIFKFE FNQNISQISL | |
| | LEWELDKIQN EDIKKNEKQV EVIKNYFDSV MSVYKMTKYF SLEKWKKRVE | |
| | LDTDNNFYND FNEYLEGFEI WKDYNLVRNY ITKKQVNTDK IKLNEDNSQF | |
| | LTWWDKDKEN ERLGIILRRE WKYYLWILKK WNTLNFGDYL QKEWEIFYEK | |
| | MNYKQLNNVY RQLPRLLFPL TKKLNELKWD ELKKYLSKYI QNFWYNEEIA | |
| | QIKIEFDIFQ ESKEKWEKFD IDKLRKLIEY YKKWVLALYS DLYDLEFIKY | |
| | KNYDDLSIFY SDVEKKMYNL NFTKIDKSLI DGKVKSWELY LFQIYNKDES | |
| | ESKKEWSTEN IHTKYFKLLF NEKNLQNLVV KLSWWADIFF RDKTENLKFK | |
| | KDKNGQEILD HRRFSQDKIM FHISITLNAN CWDKYWENQY VNEYMNKERD | |
| | IKIIWIDRWE KHLAYYCVID KSWKIFNNEI WTLNELNWVN YLEKLEKIES | |
| | SRKDSRISWW EIENIKELKN GYISQVINKL TELIVKYNAI IVFEDLNIWE | |
| | KRWRQKIEKQ IYQKLELALA KKLNYLTQKD KKDDEILWNL KALQLVPKVN | |
| | DYQDIWNYKQ SWIMFYVRAN YTSVTCPNCW LRKNLYISNS ATKENQKKSL | |
| | NSIAIKYNDW KFSFSYEIDD KSWKQKQSLN KKKFIVYSDI ERFVYSPLEK | |
| | LTKVIDVNKK LLELERDENL SLDINKQIQE KDLDSVFFKS LTHLENLILQ | |
| | LRNSDSKDNK DYISCPSCYY HSNNWLQWFE ENWDANWAYN IARKGIILLD | |
| | RIRKNQEKPD LYVSDIDWDN FVQSNQFPNT IIPIQNIEKQ VPLNIKI | |
| WP_018359861.1 | MKTQHFFEDF TSLYSLSKTI RFELKPIGKT LENIKKNGLI RRDEQRLDDY | (SEQ |
| type V | EKLKKVIDEY HEDFIANILS SFSFSEEILQ SYIQNLSESE ARAKIEKTMR | ID |
| CRISPR- | DTLAKAFSED ERYKSIFKKE LVKKDIPVWC PAYKSLCKKF DNFTTSLVPF | NO: |
| associated | HENRKNLYTS NEITASIPYR IVHVNLPKFI QNIEALCELQ KKMGADLYLE | 151) |
| protein Cpf1 | MMENLRNVWP SFVKTPDDLC NLKTYNHLMV QSSISEYNRF VGGYSTEDGT | |
| [Porphyromonas | KHQGINEWIN IYRQRNKEMR LPGLVFLHKQ ILAKVDSSSF ISDTLENDDQ | |
| macacae] | VFCVLRQFRK LFWNTVSSKE DDAASLKDLF CGLSGYDPEA IYVSDAHLAT | |
| | ISKNIFDRWN YISDAIRRKT EVLMPRKKES VERYAEKISK QIKKRQSYSL | |
| | AELDDLLAHY SEESLPAGES LLSYFTSLGG QKYLVSDGEV ILYEEGSNIW | |
| | DEVLIAFRDL QVILDKDFTE KKLGKDEEAV SVIKKALDSA LRLRKFEDLL | |
| | SGTGAEIRRD SSFYALYTDR MDKLKGLLKM YDKVRNYLTK KPYSIEKEKL | |
| | HFDNPSLLSG WDKNKELNNL SVIFRQNGYY YLGIMTPKGK NLFKTLPKLG | |
| | AEEMFYEKME YKQIAEPMLM LPKVFFPKKT KPAFAPDQSV VDIYNKKTEK | |
| | TGQKGENKKD LYRLIDFYKE ALTVHEWKLE NESESPTEQY RNIGEFFDEV | |
| | REQAYKVSMV NVPASYIDEA VENGKLYLFQ IYNKDESPYS KGIPNLHTLY | |
| | WKALFSEQNQ SRVYKLCGGG ELFYRKASLH MQDTTVHPKG ISIHKKNLNK | |
| | KGETSLENYD LVKDKRFTED KFFFHVPISI NYKNKKITNV NQMVRDYIAQ | |
| | NDDLQIIGID RGERNLLYIS RIDTRGNLLE QFSLNVIESD KGDLRTDYQK | |
| | ILGDREQERL RRRQEWKSIE SIKDLKDGYM SQVVHKICNM VVEHKAIVVL | |
| | ENLNLSFMKG RKKVEKSVYE KFERMLVDKL NYLVVDKKNL SNEPGGLYAA | |
| | YQLTNPLESF EELHRYPQSG ILFFVDPWNT SLTDPSTGFV NLLGRINYTN | |
| | VGDARKFFDR FNAIRYDGKG NILFDLDLSR FDVRVETQRK LWTLTTFGSR | |
| | IAKSKKSGKW MVERIENLSL CFLELFEQEN IGYRVEKDLK KAILSQDRKE | |
| | FYVRLIYLEN LMMQIRNSDG EEDYILSPAL NEKNLQFDSR LIEAKDLPVD | |
| | ADANGAYNVA RKGLMVVQRI KRGDHESIHR IGRAQWLRYV QEGIVE | |
| WP_013282991 | MLLYENYTKR NQITKSLRLE LRPQGKTLRN IKELNLLEQD KAIYALLERL | (SEQ |
| type V CRISPR- | KPVIDEGIKD IARDTLKNCE LSFEKLYEHF LSGDKKAYAK ESERLKKEIV | ID |
| associated | KTLIKNLPEG IGKISEINSA KYLNGVLYDE IDKTHKDSEE KQNILSDILE | NO: |
| protein Cpf1 | TKGYLALFSK FLTSRITTLE QSMPKRVIEN FEIYAANIPK MQDALERGAV | 152) |
| [Butyrivibrio | SFAIEYESIC SVDYYNQILS QEDIDSYNRL ISGIMDEDGA KEKGINQTIS | |
| proteoclasticus] | EKNIKIKSEH LEEKPFRILK QLHKQILEER EKAFTIDHID SDEEVVQVTK | |
| | EAFEQTKEQW ENIKKINGFY AKDPGDITLF IVVGPNQTHV LSQLIYGEHD | |
| | RIRLLLEEYE KNTLEVLPRR TKSEKARYDK FVNAVPKKVA KESHTEDGLQ | |
| | KMTGDDRLFI LYRDELARNY MRIKEAYGTF ERDILKSRRG IKGNRDVQES | |
| | LVSFYDELTK FRSALRIINS GNDEKADPIF YNTEDGIFEK ANRTYKAENL | |
| | CRNYVTKSPA DDARIMASCL GTPARLRTHW WNGEENFAIN DVAMIRRGDE | |
| | YYYFVLTPDV KPVDLKTKDE TDAQIFVQRK GAKSELGLPK ALFKCILEPY | |
| | FESPEHKNDK NCVIEEYVSK PLTIDRRAYD IFKNGTFKKT NIGIDGLTEE | |
| | KFKDDCRYLI DVYKEFIAVY TRYSCENMSG LKRADEYNDI GEFFSDVDTR | |
| | LCTMEWIPVS FERINDMVDK KEGLLELVRS MFLYNRPRKP YERTFIQLES | |
| | DSNMEHTSML LNSRAMIQYR AASLPRRVTH KKGSILVALR DSNGEHIPMH | |
| | IREAIYKMKN NFDISSEDFI MAKAYLAEHD VAIKKANEDI IRNRRYTEDK | |
| | FFLSLSYTKN ADISARTLDY INDKVEEDTQ DSRMAVIVTR NLKDLTYVAV | |
| | VDEKNNVLEE KSLNEIDGVN YRELLKERTK IKYHDKTRLW QYDVSSKGLK | |
| | EAYVELAVTQ ISKLATKYNA VVVVESMSST FKDKESFLDE QIFKAFEARL | |
| | CARMSDLSFN TIKEGEAGSI SNPIQVSNNN GNSYQDGVIY FLNNAYTRTL | |
| | CPDTGFVDVF DKTRLITMQS KRQFFAKMKD IRIDDGEMLE TENLEEYPTK | |
| | RLLDRKEWTV KIAGDGSYFD KDKGEYVYVN DIVREQIIPA LLEDKAVEDG | |
| | NMAEKFLDKT AISGKSVELI YKWFANALYG IITKKDGEKI YRSPITGTEI | |
| | DVSKNTTYNF GKKFMFKQEY RGDGDFLDAF LNYMQAQDIA V | |
| WP_048112740.1 | MNNYDEFTKL YPIQKTIRFE LKPQGRTMEH LETENFFEED RDRAEKYKIL | (SEQ |
| type V CRISPR- | KEAIDEYHKK FIDEHLTNMS LDWNSLKQIS EKYYKSREEK DKKVELSEQK | ID |
| associated | RMRQEIVSEF KKDDREKDLF SKKLESELLK EEIYKKGNHQ EIDALKSEDK | NO: |
| protein Cpf1 | FSGYFIGLHE NRKNMYSDGD EITAISNRIV NENFPKELDN LQKYQEARKK | 153) |
| [Candidatus | YPEWIIKAES ALVAHNIKMD EVESLEYENK VLNQEGIQRY NLALGGYVTK | |
| Methanoplasma | SGEKMMGLND ALNLAHQSEK SSKGRIHMTP LFKQILSEKE SFSYIPDVET | |
| termitum] | EDSQLLPSIG GFFAQIENDK DGNIFDRALE LISSYAEYDT ERIYIRQADI | |
| | NRVSNVIFGE WGTLGGLMRE YKADSINDIN LERTCKKVDK WLDSKEFALS | |
| | DVLEAIKRTG NNDAFNEYIS KMRTAREKID AARKEMKFIS EKISGDEESI | |
| | HIIKTLLDSV QQFLHFFNLF KARQDIPLDG AFYAEFDEVH SKLFAIVPLY | |
| | NKVRNYLTKN NLNTKKIKLN FKNPTLANGW DQNKVYDYAS LIFLRDGNYY | |
| | LGIINPKRKK NIKFEQGSGN GPFYRKMVYK QIPGPNKNLP RVFLTSTKGK | |
| | KEYKPSKEII EGYEADKHIR GDKEDLDFCH KLIDFFKESI EKHKDWSKEN | |
| | FYFSPTESYG DISEFYLDVE KQGYRMHFEN ISAETIDEYV EKGDLFLFQI | |
| | YNKDFVKAAT GKKDMHTIYW NAAFSPENLQ DVVVKLNGEA ELFYRDKSDI | |
| | KEIVHREGEI LVNRTYNGRT PVPDKIHKKL TDYHNGRTKD LGEAKEYLDK | |
| | VRYFKAHYDI TKDRRYLNDK IYFHVPLTLN FKANGKKNLN KMVIEKELSD | |
| | EKAHIIGIDR GERNLLYYSI IDRSGKIIDQ QSLNVIDGED YREKLNQREI | |
| | EMKDARQSWN AIGKIKDLKE GYLSKAVHEI TKMAIQYNAI VVMEELNYGE | |
| | KRGREKVEKQ IYQKFENMLI DKMNYLVFKD APDESPGGVL NAYQLTNPLE | |
| | SFAKLGKQTG ILFYVPAAYT SKIDPTTGFV NLENTSSKIN AQERKEFLQK | |
| | FESISYSAKD GGIFAFAFDY RKFGTSKTDH KNVWTAYTNG ERMRYIKEKK | |
| | RNELFDPSKE IKEALTSSGI KYDGGQNILP DILRSNNNGL IYTMYSSFIA | |
| | AIQMRVYDGK EDYIISPIKN SKGEFFRTDP KRRELPIDAD ANGAYNIALR | |
| | GELTMRAIAE KEDPDSEKMA KLELKHKDWF EFMQTRGD | |
| WP_027407524.1 | MVAFIDEFVG QYPVSKTLRF EARPVPETKK WLESDQCSVL ENDQKRNEYY | (SEQ |
| type V CRISPR- | GVLKELLDDY YRAYIEDALT SFTLDKALLE NAYDLYCNRD TNAFSSCCEK | ID |
| associated | LRKDLVKAFG NLKDYLLGSD QLKDLVKLKA KVDAPAGKGK KKIEVDSRLI | NO: |
| protein Cpf1 | NWLNNNAKYS AEDREKYIKA IESFEGFVTY LTNYKQAREN MESSEDKSTA | 154) |
| [Anaerovibrio | IAFRVIDQNM VTYFGNIRIY EKIKAKYPEL YSALKGFEKF FSPTAYSEIL | |
| sp. RM50] | SQSKIDEYNY QCIGRPIDDA DEKGVNSLIN EYRQKNGIKA RELPVMSMLY | |
| | KQILSDRDNS FMSEVINRNE EAIECAKNGY KVSYALENEL LQLYKKIFTE | |
| | DNYGNIYVKT QPLTELSQAL FGDWSILRNA LDNGKYDKDI INLAELEKYF | |
| | SEYCKVLDAD DAAKIQDKEN LKDYFIQKNA LDATLPDLDK ITQYKPHLDA | |
| | MLQAIRKYKL FSMYNGRKKM DVPENGIDES NEFNAIYDKL SEFSILYDRI | |
| | RNFATKKPYS DEKMKLSFNM PTMLAGWDYN NETANGCFLF IKDGKYFLGV | |
| | ADSKSKNIFD FKKNPHLLDK YSSKDIYYKV KYKQVSGSAK MLPKVVFAGS | |
| | NEKIFGHLIS KRILEIREKK LYTAAAGDRK AVAEWIDEMK SAIAIHPEWN | |
| | EYFKFKFKNT AEYDNANKFY EDIDKQTYSL EKVEIPTEYI DEMVSQHKLY | |
| | LFQLYTKDES DKKKKKGTDN LHTMYWHGVF SDENLKAVTE GTQPIIKLNG | |
| | EAEMFMRNPS IEFQVTHEHN KPIANKNPLN TKKESVENYD LIKDKRYTER | |
| | KFYFHCPITL NFRADKPIKY NEKINREVEN NPDVCIIGID RGERHLLYYT | |
| | VINQTGDILE QGSLNKISGS YTNDKGEKVN KETDYHDLLD RKEKGKHVAQ | |
| | QAWETIENIK ELKAGYLSQV VYKLTQLMLQ YNAVIVLENL NVGFKRGRTK | |
| | VEKQVYQKFE KAMIDKLNYL VEKDRGYEMN GSYAKGLQLT DKFESEDKIG | |
| | KQTGCIYYVI PSYTSHIDPK TGFVNLLNAK LRYENITKAQ DTIRKEDSIS | |
| | YNAKADYFEF AFDYRSFGVD MARNEWVVCT CGDLRWEYSA KTRETKAYSV | |
| | TDRLKELFKA HGIDYVGGEN LVSHITEVAD KHELSTLLFY LRLVLKMRYT | |
| | VSGTENENDF ILSPVEYAPG KFFDSREATS TEPMNADANG AYHIALKGLM | |
| | TIRGIEDGKL HNYGKGGENA AWFKFMQNQE YKNNG | |
| WP_044910712.1 | MDYGNGQFER RAPLTKTITL RLKPIGETRE TIREQKLLEQ DAAFRKLVET | (SEQ |
| type V | VTPIVDDCIR KIADNALCHF GTEYDESCLG NAISKNDSKA IKKETEKVEK | ID |
| CRISPR- | LLAKVLTENL PDGLRKVNDI NSAAFIQDTL TSFVQDDADK RVLIQELKGK | NO: |
| associated | TVLMQRELTT RITALTVWLP DRVFENFNIF IENAEKMRIL LDSPLNEKIM | 155) |
| protein Cpf1 | KEDPDAEQYA SLEFYGQCLS QKDIDSYNLI ISGIYADDEV KNPGINEIVK | |
| [Lachnospiraceae | EYNQQIRGDK DESPLPKLKK LHKQILMPVE KAFFVRVLSN DSDARSILEK | |
| bacterium | ILKDTEMLPS KIIEAMKEAD AGDIAVYGSR LHELSHVIYG DHGKLSQIIY | |
| MC2017] | DKESKRISEL METLSPKERK ESKKRLEGLE EHIRKSTYTF DELNRYAEKN | |
| | VMAAYIAAVE ESCAEIMRKE KDLRTLLSKE DVKIRGNRHN TLIVKNYENA | |
| | WTVERNLIRI LRRKSEAEID SDFYDVLDDS VEVLSLTYKG ENLCRSYITK | |
| | KIGSDLKPEI ATYGSALRPN SRWWSPGEKF NVKFHTIVRR DGRLYYFILP | |
| | KGAKPVELED MDGDIECLQM RKIPNPTIFL PKLVFKDPEA FFRDNPEADE | |
| | FVFLSGMKAP VTITRETYEA YRYKLYTVGK LRDGEVSEEE YKRALLQVLT | |
| | AYKEFLENRM IYADLNFGFK DLEEYKDSSE FIKQVETHNT FMCWAKVSSS | |
| | QLDDLVKSGN GLLFEIWSER LESYYKYGNE KVLRGYEGVL LSILKDENLV | |
| | SMRTLLNSRP MLVYRPKESS KPMVVHRDGS RVVDREDKDG KYIPPEVHDE | |
| | LYRFENNLLI KEKLGEKARK ILDNKKVKVK VLESERVKWS KFYDEQFAVT | |
| | FSVKKNADCL DTTKDLNAEV MEQYSESNRL ILIRNTTDIL YYLVLDKNGK | |
| | VLKQRSLNII NDGARDVDWK ERFRQVTKDR NEGYNEWDYS RTSNDLKEVY | |
| | LNYALKEIAE AVIEYNAILI IEKMSNAFKD KYSELDDVTF KGFETKLLAK | |
| | LSDLHERGIK DGEPCSFTNP LQLCQNDSNK ILQDGVIFMV PNSMTRSLDP | |
| | DTGFIFAIND HNIRTKKAKL NFLSKEDQLK VSSEGCLIMK YSGDSLPTHN | |
| | TDNRVWNCCC NHPITNYDRE TKKVEFIEEP VEELSRVLEE NGIETDTELN | |
| | KLNERENVPG KVVDAIYSLV LNYLRGTVSG VAGQRAVYYS PVTGKKYDIS | |
| | FIQAMNLNRK CDYYRIGSKE RGEWTDEVAQ LIN | |
| WP_081834226 | MTMDYGNGQF ERRAPLTKTI TLRLKPIGET RETIREQKLL EQDAAFRKLV | (SEQ |
| type V CRISPR- | ETVTPIVDDC IRKIADNALC HFGTEYDESC LGNAISKNDS KAIKKETEKV | ID |
| associated | EKLLAKVLTE NLPDGLRKVN DINSAAFIQD TLTSFVQDDA DKRVLIQELK | NO: |
| protein Cpf1 | GKTVLMQRFL TTRITALTVW LPDRVFENEN IFIENAEKMR ILLDSPLNEK | 156) |
| [Lachnospiraceae | IMKFDPDAEQ YASLEFYGQC LSQKDIDSYN LIISGIYADD EVKNPGINEI | |
| bacterium | VKEYNQQIRG DKDESPLPKL KKLHKQILMP VEKAFFVRVL SNDSDARSIL | |
| MC2017]. | EKILKDTEML PSKITEAMKE ADAGDIAVYG SRLHELSHVI YGDHGKLSQI | |
| | IYDKESKRIS ELMETLSPKE RKESKKRLEG LEEHIRKSTY TFDELNRYAE | |
| | KNVMAAYIAA VEESCAEIMR KEKDLRTLLS KEDVKIRGNR HNTLIVKNYF | |
| | NAWTVERNLI RILRRKSEAE IDSDFYDVLD DSVEVLSLTY KGENLCRSYI | |
| | TKKIGSDLKP EIATYGSALR PNSRWWSPGE KENVKFHTIV RRDGRLYYFI | |
| | LPKGAKPVEL EDMDGDIECL QMRKIPNPTI FLPKLVEKDP EAFFRDNPEA | |
| | DEFVELSGMK APVTITRETY EAYRYKLYTV GKLRDGEVSE EEYKRALLQV | |
| | LTAYKEFLEN RMIYADLNFG FKDLEEYKDS SEFIKQVETH NTFMCWAKVS | |
| | SSQLDDLVKS GNGLLFEIWS ERLESYYKYG NEKVLRGYEG VLLSILKDEN | |
| | LVSMRTLLNS RPMLVYRPKE SSKPMVVHRD GSRVVDREDK DGKYIPPEVH | |
| | DELYRFENNL LIKEKLGEKA RKILDNKKVK VKVLESERVK WSKFYDEQFA | |
| | VTFSVKKNAD CLDTTKDLNA EVMEQYSESN RLILIRNTTD ILYYLVLDKN | |
| | GKVLKQRSLN IINDGARDVD WKERFRQVTK DRNEGYNEWD YSRTSNDLKE | |
| | VYLNYALKEI AEAVIEYNAI LIIEKMSNAF KDKYSFLDDV TFKGFETKLL | |
| | AKLSDLHFRG IKDGEPCSFT NPLQLCQNDS NKILQDGVIF MVPNSMTRSL | |
| | DPDTGFIFAI NDHNIRTKKA KLNFLSKEDQ LKVSSEGCLI MKYSGDSLPT | |
| | HNTDNRVWNC CCNHPITNYD RETKKVEFIE EPVEELSRVL EENGIETDTE | |
| | LNKLNERENV PGKVVDAIYS LVLNYLRGTV SGVAGQRAVY YSPVTGKKYD | |
| | ISFIQAMNLN RKCDYYRIGS KERGEWTDEV AQLIN | |
| WP_027216152.1 | MYYESLTKLY PIKKTIRNEL VPIGKTLENI KKNNILEADE DRKIAYIRVK | (SEQ |
| type V CRISPR- | AIMDDYHKRL INEALSGFAL IDLDKAANLY LSRSKSADDI ESFSRFQDKL | ID |
| associated | RKAIAKRLRE HENFGKIGNK DIIPLLQKLS ENEDDYNALE SFKNFYTYFE | NO: |
| protein Cpf1 | SYNDVRLNLY SDKEKSSTVA YRLINENLPR FLDNIRAYDA VQKAGITSEE | 157) |
| [Butyrivibrio | LSSEAQDGLF LVNTENNVLI QDGINTYNED IGKLNVAINL YNQKNASVQG | |
| fibrisolvens] | FRKVPKMKVL YKQILSDREE SFIDEFESDT ELLDSLESHY ANLAKYFGSN | |
| | KVQLLFTALR ESKGVNVYVK NDIAKTSFSN VVFGSWSRID ELINGEYDDN | |
| | NNRKKDEKYY DKRQKELKKN KSYTIEKIIT LSTEDVDVIG KYIEKLESDI | |
| | DDIRFKGKNF YEAVLCGHDR SKKLSKNKGA VEAIKGYLDS VKDFERDLKL | |
| | INGSGQELEK NLVVYGEQEA VLSELSGIDS LYNMTRNYLT KKPESTEKIK | |
| | LNFNKPTELD GWDYGNEEAY LGFFMIKEGN YFLAVMDANW NKEFRNIPSV | |
| | DKSDCYKKVI YKQISSPEKS IQNLMVIDGK TVKKNGRKEK EGIHSGENLI | |
| | LEELKNTYLP KKINDIRKRR SYLNGDTFSK KDLTEFIGYY KQRVIEYYNG | |
| | YSFYFKSDDD YASFKEFQED VGRQAYQISY VDVPVSFVDD LINSGKLYLF | |
| | RVYNKDFSEY SKGRLNLHTL YFKMLEDERN LKNVVYKLNG QAEVFYRPSS | |
| | IKKEELIVHR AGEEIKNKNP KRAAQKPTRR LDYDIVKDRR YSQDKFMLHT | |
| | SIIMNFGAEE NVSENDIVNG VLRNEDKVNV IGIDRGERNL LYVVVIDPEG | |
| | KILEQRSLNC ITDSNLDIET DYHRLLDEKE SDRKIARRDW TTIENIKELK | |
| | AGYLSQVVHI VAELVLKYNA IICLEDLNFG FKRGRQKVEK QVYQKFEKML | |
| | IDKLNYLVMD KSREQLSPEK ISGALNALQL TPDFKSFKVL GKQTGIIYYV | |
| | PAYLTSKIDP MTGFANLFYV KYENVDKAKE FFSKEDSIKY NKDGKNWNTK | |
| | GYFEFAFDYK KFTDRAYGRV SEWTVCTVGE RIIKFKNKEK NNSYDDKVID | |
| | LTNSLKELED SYKVTYESEV DLKDAILAID DPAFYRDLTR RLQQTLQMRN | |
| | SSCDGSRDYI ISPVKNSKGE FFCSDNNDDT TPNDADANGA FNIARKGLWV | |
| | LNEIRNSEEG SKINLAMSNA QWLEYAQDNT I | |
| WP_016301126.1 | MHENNGKIAD NFIGIYPVSK TLRFELKPVG KTQEYIEKHG ILDEDLKRAG | (SEQ |
| type V | DYKSVKKIID AYHKYFIDEA LNGIQLDGLK NYYELYEKKR DNNEEKEFQK | ID |
| CRISPR- | IQMSLRKQIV KRFSEHPQYK YLFKKELIKN VLPEFTKDNA EEQTLVKSFQ | NO: |
| associated | EFTTYFEGFH QNRKNMYSDE EKSTAIAYRV VHQNLPKYID NMRIFSMILN | 158) |
| protein Cpf1 | TDIRSDLTEL FNNLKTKMDI TIVEEYFAID GENKVVNQKG IDVYNTILGA | |
| [Lachnospiraceae | FSTDDNTKIK GLNEYINLYN QKNKAKLPKL KPLFKQILSD RDKISFIPEQ | |
| bacterium | FDSDTEVLEA VDMFYNRLLQ FVIENEGQIT ISKLLTNFSA YDLNKIYVKN | |
| COE1] | DTTISAISND LEDDWSYISK AVRENYDSEN VDKNKRAAAY EEKKEKALSK | |
| | IKMYSIEELN FFVKKYSCNE CHIEGYFERR ILEILDKMRY AYESCKILHD | |
| | KGLINNISLC QDRQAISELK DELDSIKEVQ WLLKPLMIGQ EQADKEEAFY | |
| | TELLRIWEEL EPITLLYNKV RNYVTKKPYT LEKVKLNFYK STLLDGWDKN | |
| | KEKDNLGIIL LKDGQYYLGI MNRRNNKIAD DAPLAKTDNV YRKMEYKLLT | |
| | KVSANLPRIF LKDKYNPSEE MLEKYEKGTH LKGENFCIDD CRELIDEFKK | |
| | GIKQYEDWGQ FDFKFSDTES YDDISAFYKE VEHQGYKITF RDIDETYIDS | |
| | LVNEGKLYLF QIYNKDESPY SKGTKNLHTL YWEMLESQQN LQNIVYKLNG | |
| | NAEIFYRKAS INQKDVVVHK ADLPIKNKDP QNSKKESMED YDIIKDKRFT | |
| | CDKYQFHVPI TMNFKALGEN HFNRKVNRLI HDAENMHIIG IDRGERNLIY | |
| | LCMIDMKGNI VKQISLNEII SYDKNKLEHK RNYHQLLKTR EDENKSARQS | |
| | WQTIHTIKEL KEGYLSQVIH VITDLMVEYN AIVVLEDLNE GFKQGRQKFE | |
| | RQVYQKFEKM LIDKLNYLVD KSKGMDEDGG LLHAYQLTDE FKSFKQLGKQ | |
| | SGFLYYIPAW NTSKLDPTTG FVNLFYTKYE SVEKSKEFIN NFTSILYNQE | |
| | REYFEFLFDY SAFTSKAEGS RLKWTVCSKG ERVETYRNPK KNNEWDTQKI | |
| | DLTFELKKLF NDYSISLLDG DLREQMGKID KADFYKKEMK LFALIVQMRN | |
| | SDEREDKLIS PVLNKYGAFF ETGKNERMPL DADANGAYNI ARKGLWIIEK | |
| | IKNTDVEQLD KVKLTISNKE WLQYAQEHIL | |
| WP_035635841.1 | MSKLEKFTNC YSLSKTLRFK AIPVGKTQEN IDNKRLLVED EKRAEDYKGV | (SEQ |
| type V CRISPR- | KKLLDRYYLS FINDVLHSIK LKNLNNYISL FRKKTRTEKE NKELENLEIN | ID |
| associated | LRKEIAKAFK GNEGYKSLFK KDIIETILPE FLDDKDEIAL VNSENGETTA | NO: |
| protein Cpf1 | FTGFEDNREN MESEEAKSTS IAFRCINENL TRYISNMDIF EKVDAIFDKH | 159) |
| [Lachnospiraceae | EVQEIKEKIL NSDYDVEDFF EGEFFNFVLT QEGIDVYNAI IGGFVTESGE | |
| bacterium | KIKGLNEYIN LYNQKTKQKL PKFKPLYKQV LSDRESLSFY GEGYTSDEEV | |
| ND2006] | LEVFRNTLNK NSEIFSSIKK LEKLFKNFDE YSSAGIFVKN GPAISTISKD | |
| | IFGEWNVIRD KWNAEYDDIH LKKKAVVTEK YEDDRRKSFK KIGSESLEQL | |
| | QEYADADLSV VEKLKEIIIQ KVDEIYKVYG SSEKLEDADE VLEKSLKKND | |
| | AVVAIMKDLL DSVKSFENYI KAFFGEGKET NRDESFYGDF VLAYDILLKV | |
| | DHIYDAIRNY VTQKPYSKDK FKLYFQNPQF MGGWDKDKET DYRATILRYG | |
| | SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS | |
| | NAYDENFSET EKYKDIAGFY REVEEQGYKV SFESASKKEV DKLVEEGKLY | |
| | MFQIYNKDES DKSHGTPNLH TMYFKLLEDE NNHGQIRLSG GAELEMRRAS | |
| | LKKEELVVHP ANSPIANKNP DNPKKTTTLS YDVYKDKRFS EDQYELHIPI | |
| | AINKCPKNIF KINTEVRVLL KHDDNPYVIG IDRGERNLLY IVVVDGKGNI | |
| | VEQYSLNEII NNENGIRIKT DYHSLLDKKE KERFEARQNW TSIENIKELK | |
| | AGYISQVVHK ICELVEKYDA VIALEDLNSG FKNSRVKVEK QVYQKFEKML | |
| | IDKLNYMVDK KSNPCATGGA LKGYQITNKF ESFKSMSTQN GFIFYIPAWL | |
| | TSKIDPSTGF VNLLKTKYTS IADSKKFISS FDRIMYVPEE DLFEFALDYK | |
| | NFSRTDADYI KKWKLYSYGN RIRIFRNPKK NNVEDWEEVC LTSAYKELEN | |
| | KYGINYQQGD IRALLCEQSD KAFYSSFMAL MSLMLQMRNS ITGRTDVDEL | |
| | ISPVKNSDGI FYDSRNYEAQ ENAILPKNAD ANGAYNIARK VLWAIGQFKK | |
| | AEDEKLDKVK IAISNKEWLE YAQTSVKH | |
| WP_051666128.1 | MLKNVGIDRL DVEKGRKNMS KLEKFTNCYS LSKTLREKAI PVGKTQENID | (SEQ |
| type V CRISPR- | NKRLLVEDEK RAEDYKGVKK LLDRYYLSFI NDVLHSIKLK NLNNYISLER | ID |
| associated | KKTRTEKENK ELENLEINLR KEIAKAFKGN EGYKSLFKKD IIETILPEFL | NO: |
| protein Cpf1 | DDKDEIALVN SENGFTTAFT GFFDNRENMF SEEAKSTSIA FRCINENLTR | 160) |
| [Lachnospiraceae | YISNMDIFEK VDAIFDKHEV QEIKEKILNS DYDVEDFFEG EFFNFVLTQE | |
| bacterium | GIDVYNAIIG GFVTESGEKI KGLNEYINLY NQKTKQKLPK FKPLYKQVLS | |
| ND2006] | DRESLSFYGE GYTSDEEVLE VFRNTLNKNS EIFSSIKKLE KLEKNEDEYS | |
| | SAGIFVKNGP AISTISKDIF GEWNVIRDKW NAEYDDIHLK KKAVVTEKYE | |
| | DDRRKSFKKI GSFSLEQLQE YADADLSVVE KLKEIIIQKV DEIYKVYGSS | |
| | EKLFDADFVL EKSLKKNDAV VAIMKDLLDS VKSFENYIKA FFGEGKETNR | |
| | DESFYGDFVL AYDILLKVDH IYDAIRNYVT QKPYSKDKFK LYFQNPQFMG | |
| | GWDKDKETDY RATILRYGSK YYLAIMDKKY AKCLQKIDKD DVNGNYEKIN | |
| | YKLLPGPNKM LPKVFFSKKW MAYYNPSEDI QKIYKNGTFK KGDMFNLNDC | |
| | HKLIDFFKDS ISRYPKWSNA YDFNFSETEK YKDIAGFYRE VEEQGYKVSF | |
| | ESASKKEVDK LVEEGKLYMF QIYNKDESDK SHGTPNLHTM YFKLLEDENN | |
| | HGQIRLSGGA ELFMRRASLK KEELVVHPAN SPIANKNPDN PKKTTTLSYD | |
| | VYKDKRFSED QYELHIPIAI NKCPKNIFKI NTEVRVLLKH DDNPYVIGID | |
| | RGERNLLYIV VVDGKGNIVE QYSLNEIINN ENGIRIKTDY HSLLDKKEKE | |
| | RFEARQNWTS IENIKELKAG YISQVVHKIC ELVEKYDAVI ALEDLNSGFK | |
| | NSRVKVEKQV YQKFEKMLID KLNYMVDKKS NPCATGGALK GYQITNKFES | |
| | FKSMSTQNGF IFYIPAWLTS KIDPSTGFVN LLKTKYTSIA DSKKFISSED | |
| | RIMYVPEEDL FEFALDYKNF SRTDADYIKK WKLYSYGNRI RIFRNPKKNN | |
| | VEDWEEVCLT SAYKELFNKY GINYQQGDIR ALLCEQSDKA FYSSEMALMS | |
| | LMLQMRNSIT GRTDVDFLIS PVKNSDGIFY DSRNYEAQEN AILPKNADAN | |
| | GAYNIARKVL WAIGQFKKAE DEKLDKVKIA ISNKEWLEYA QTSVKH | |
| WP_015504779.1 | MDAKEFTGQY PLSKTLRFEL RPIGRTWDNL EASGYLAEDR HRAECYPRAK | (SEQ |
| type V | ELLDDNHRAF LNRVLPQIDM DWHPIAEAFC KVHKNPGNKE LAQDYNLQLS | ID |
| CRISPR- | KRRKEISAYL QDADGYKGLF AKPALDEAMK IAKENGNESD IEVLEAFNGE | NO: |
| associated | SVYFTGYHES RENIYSDEDM VSVAYRITED NEPRFVSNAL IFDKLNESHP | 161) |
| protein Cpf1 | DIISEVSGNL GVDDIGKYFD VSNYNNFLSQ AGIDDYNHII GGHTTEDGLI | |
| [Candidatus | QAFNVVLNLR HQKDPGFEKI QFKQLYKQIL SVRTSKSYIP KQFDNSKEMV | |
| Methanomethylophilus | DCICDYVSKI EKSETVERAL KLVRNISSED LRGIFVNKKN LRILSNKLIG | |
| alvus] | DWDAIETALM HSSSSENDKK SVYDSAEAFT LDDIFSSVKK FSDASAEDIG | |
| | NRAEDICRVI SETAPFINDL RAVDLDSLND DGYEAAVSKI RESLEPYMDL | |
| | FHELEIFSVG DEFPKCAAFY SELEEVSEQL IEIIPLENKA RSFCTRKRYS | |
| | TDKIKVNLKF PTLADGWDLN KERDNKAAIL RKDGKYYLAI LDMKKDLSSI | |
| | RTSDEDESSF EKMEYKLLPS PVKMLPKIFV KSKAAKEKYG LTDRMLECYD | |
| | KGMHKSGSAF DLGFCHELID YYKRCIAEYP GWDVEDEKER ETSDYGSMKE | |
| | FNEDVAGAGY YMSLRKIPCS EVYRLLDEKS IYLFQIYNKD YSENAHGNKN | |
| | MHTMYWEGLF SPQNLESPVF KLSGGAELFF RKSSIPNDAK TVHPKGSVLV | |
| | PRNDVNGRRI PDSIYRELTR YFNRGDCRIS DEAKSYLDKV KTKKADHDIV | |
| | KDRRFTVDKM MFHVPIAMNE KAISKPNLNK KVIDGIIDDQ DLKIIGIDRG | |
| | ERNLIYVTMV DRKGNILYQD SLNILNGYDY RKALDVREYD NKEARRNWTK | |
| | VEGIRKMKEG YLSLAVSKLA DMIIENNAII VMEDLNHGFK AGRSKIEKQV | |
| | YQKFESMLIN KLGYMVLKDK SIDQSGGALH GYQLANHVTT LASVGKQCGV | |
| | IFYIPAAFTS KIDPTTGFAD LFALSNVKNV ASMREFFSKM KSVIYDKAEG | |
| | KFAFTEDYLD YNVKSECGRT LWTVYTVGER FTYSRVNREY VRKVPTDIIY | |
| | DALQKAGISV EGDLRDRIAE SDGDTLKSIF YAFKYALDMR VENREEDYIQ | |
| | SPVKNASGEF FCSKNAGKSL PQDSDANGAY NIALKGILQL RMLSEQYDPN | |
| | AESIRLPLIT NKAWLTEMQS GMKTWKN | |
| WP_044910713.1 | MGLYDGFVNR YSVSKTLRFE LIPQGRTREY IETNGILSDD EERAKDYKTI | (SEQ |
| type V CRISPR- | KRLIDEYHKD YISRCLKNVN ISCLEEYYHL YNSSNRDKRH EELDALSDQM | ID |
| associated | RGEIASFLTG NDEYKEQKSR DIIINERIIN FASTDEELAA VKRERKFTSY | NO: |
| protein Cpf1 | FTGFFTNREN MYSAEKKSTA IAHRIIDVNL PKYVDNIKAF NTAIEAGVED | 162) |
| [Lachnospiraceae | IAEFESNFKA ITDEHEVSDL LDITKYSRFI RNEDIIIYNT LLGGISMKDE | |
| bacterium | KIQGLNELIN LHNQKHPGKK VPLLKVLYKQ ILGDSQTHSF VDDQFEDDQQ | |
| MC2017] | VINAVKAVTD TFSETLLGSL KIIINNIGHY DLDRIYIKAG QDITTLSKRA | |
| | LNDWHIITEC LESEYDDKFP KNKKSDTYEE MRNRYVKSFK SFSIGRLNSL | |
| | VTTYTEQACF LENYLGSFGG DTDKNCLTDF TNSLMEVEHL LNSEYPVTNR | |
| | LITDYESVRI LKRLLDSEME VIHFLKPLLG NGNESDKDLV FYGEFEAEYE | |
| | KLLPVIKVYN RVRNYLTRKP FSTEKIKLNF NSPTLLCGWS QSKEKEYMGV | |
| | ILRKDGQYYL GIMTPSNKKI FSEAPKPDED CYEKMVLRYI PHPYQMLPKV | |
| | FFSKSNIAFF NPSDEILRIK KQESFKKGKS FNRDDCHKFI DFYKDSINRH | |
| | EEWRKENFKF SDTDSYEDIS RFYKEVENQA FSMSFTKIPT VYIDSLVDEG | |
| | KLYLFKLHNK DFSEHSKGKP NLHTVYWNAL FSEYNLQNTV YQLNGSAEIF | |
| | FRKASIPENE RVIHKKNVPI TRKVAELNGK KEVSVFPYDI IKNRRYTVDK | |
| | FQFHVPLKMN FKADEKKRIN DDVIEAIRSN KGIHVIGIDR GERNLLYLSL | |
| | INEEGRIIEQ RSLNIIDSGE GHTQNYRDLL DSREKDREKA RENWQEIQEI | |
| | KDLKTGYLSQ AIHTITKWMK EYNAIIVLED LNDRFTNGRK KVEKQVYQKF | |
| | EKMLIDKLNY YVDKDEEFDR MGGTHRALQL TEKFESFQKL GRQTGFIFYV | |
| | PAWNTSKLDP TTGFVDLLYP KYKSVDATKD FIKKEDFIRF NSEKNYFEFG | |
| | LHYSNFTERA IGCRDEWILC SYGNRIVNER NAAKNNSWDY KEIDITKQLL | |
| | DLFEKNGIDV KQENLIDSIC EMKDKPFFKS LIANIKLILQ IRNSASGTDI | |
| | DYMISPAMND RGEFFDTRKG LQQLPLDADA NGAYNIAKKG LWIVDQIRNT | |
| | TGNNVKMAMS NREWMHFAQE SRLA | |
| KKQ36153.1 | MKNVFGGFTN LYSLTKTLRF ELKPTSKTQK LMKRNNVIQT DEEIDKLYHD | (SEQ |
| hypothetical | EMKPILDEIH RRFINDALAQ KIFISASLDN FLKVVKNYKV ESAKKNIKQN | ID |
| protein | QVKLLQKEIT IKTLGLRREV VSGFITVSKK WKDKYVGLGI KLKGDGYKVL | NO: |
| US52_C0007G0 | TEQAVLDILK IEFPNKAKYI DKFRGFWTYF SGENENRKNY YSEEDKATSI | 163) |
| 008 [candidate | ANRIVNENLS RYIDNIIAFE EILQKIPNLK KFKQDLDITS YNYYLNQAGI | |
| division WS6 | DKYNKIIGGY IVDKDKKIQG INEKVNLYTQ QTKKKLPKLK FLFKQIGSER | |
| bacterium | KGFGIFEIKE GKEWEQLGDL FKLQRTKINS NGREKGLEDS LRTMYREFED | |
| GW2011_GWA | EIKRDSNSQA RYSLDKIYEN KASVNTISNS WFTNWNKFAE LLNIKEDKKN | |
| 2_37_6] | GEKKIPEQIS IEDIKDSLSI IPKENLEELF KLTNREKHDR TRFFGSNAWV | |
| | TELNIWQNEI EESENKLEEK EKDEKKNAAI KFQKNNLVQK NYIKEVCDRM | |
| | LAIERMAKYH LPKDSNLSRE EDFYWIIDNL SEQREIYKYY NAFRNYISKK | |
| | PYNKSKMKLN FENGNLLGGW SDGQERNKAG VILRNGNKYY LGVLINRGIF | |
| | RTDKINNEIY RTGSSKWERL ILSNLKFQTL AGKGFLGKHG VSYGNMNPEK | |
| | SVPSLQKFIR ENYLKKYPQL TEVSNTKELS KKDFDAAIKE ALKECFTMNF | |
| | INIAENKLLE AEDKGDLYLF EITNKDESGK KSGKDNIHTI YWKYLESESN | |
| | CKSPIIGLNG GAEIFFREGQ KDKLHTKLDK KGKKVEDAKR YSEDKLFFHV | |
| | SITINYGKPK NIKFRDIINQ LITSMNVNII GIDRGEKHLL YYSVIDSNGI | |
| | ILKQGSLNKI RVGDKEVDEN KKLTERANEM KKARQSWEQI GNIKNFKEGY | |
| | LSQAIHEIYQ LMIKYNAIIV LEDLNTEFKA KRLSKVEKSV YKKFELKLAR | |
| | KLNHLILKDR NTNEIGGVLK AYQLTPTIGG GDVSKFEKAK QWGMMFYVRA | |
| | NYTSTTDPVT GWRKHLYISN FSNNSVIKSF FDPTNRDTGI EIFYSGKYRS | |
| | WGFRYVQKET GKKWELFATK ELERFKYNQT TKLCEKINLY DKFEELFKGI | |
| | DKSADIYSQL CNVLDFRWKS LVYLWNLLNQ IRNVDKNAEG NKNDFIQSPV | |
| | YPFFDSRKTD GKTEPINGDA NGALNIARKG LMLVERIKNN PEKYEQLIRD | |
| | TEWDAWIQNF NKVN | |
| WP_044919442.1 | MYYESLTKQY PVSKTIRNEL IPIGKTLDNI RQNNILESDV KRKQNYEHVK | (SEQ |
| type V CRISPR- | GILDEYHKQL INEALDNCTL PSLKIAAEIY LKNQKEVSDR EDENKTQDLL | ID |
| associated | RKEVVEKLKA HENFTKIGKK DILDLLEKLP SISEDDYNAL ESFRNFYTYF | NO: |
| protein Cpf1 | TSYNKVRENL YSDKEKSSTV AYRLINENFP KELDNVKSYR FVKTAGILAD | 164) |
| [Lachnospiraceae | GLGEEEQDSL FIVETENKTL TQDGIDTYNS QVGKINSSIN LYNQKNQKAN | |
| bacterium | GFRKIPKMKM LYKQILSDRE ESFIDEFQSD EVLIDNVESY GSVLIESLKS | |
| MA2020] | SKVSAFFDAL RESKGKNVYV KNDLAKTAMS NIVFENWRTF DDLLNQEYDL | |
| | ANENKKKDDK YFEKRQKELK KNKSYSLEHL CNLSEDSCNL IENYIHQISD | |
| | DIENIIINNE TELRIVINEH DRSRKLAKNR KAVKAIKDEL DSIKVLEREL | |
| | KLINSSGQEL EKDLIVYSAH EELLVELKQV DSLYNMTRNY LTKKPESTEK | |
| | VKLNFNRSTL LNGWDRNKET DNLGVLLLKD GKYYLGIMNT SANKAFVNPP | |
| | VAKTEKVFKK VDYKLLPVPN QMLPKVFFAK SNIDFYNPSS EIYSNYKKGT | |
| | HKKGNMESLE DCHNLIDFFK ESISKHEDWS KFGFKESDTA SYNDISEFYR | |
| | EVEKQGYKLT YTDIDETYIN DLIERNELYL FQIYNKDFSM YSKGKLNLHT | |
| | LYFMMLFDQR NIDDVVYKLN GEAEVFYRPA SISEDELIIH KAGEEIKNKN | |
| | PNRARTKETS TFSYDIVKDK RYSKDKFTLH IPITMNFGVD EVKRENDAVN | |
| | SAIRIDENVN VIGIDRGERN LLYVVVIDSK GNILEQISLN SIINKEYDIE | |
| | TDYHALLDER EGGRDKARKD WNTVENIRDL KAGYLSQVVN VVAKLVLKYN | |
| | AIICLEDLNF GFKRGRQKVE KQVYQKFEKM LIDKLNYLVI DKSREQTSPK | |
| | ELGGALNALQ LTSKFKSFKE LGKQSGVIYY VPAYLTSKID PTTGFANLFY | |
| | MKCENVEKSK RFEDGEDFIR FNALENVFEF GFDYRSFTQR ACGINSKWTV | |
| | CTNGERIIKY RNPDKNNMED EKVVVVTDEM KNLFEQYKIP YEDGRNVKDM | |
| | IISNEEAEFY RRLYRLLQQT LQMRNSTSDG TRDYIISPVK NKREAYENSE | |
| | LSDGSVPKDA DANGAYNIAR KGLWVLEQIR QKSEGEKINL AMTNAEWLEY | |
| | AQTHLL | |
| WP_035798880.1 | MYYQNLTKKY PVSKTIRNEL IPIGKTLENI RKNNILESDV KRKQDYEHVK | (SEQ |
| type V | GIMDEYHKQL INEALDNYML PSLNQAAEIY LKKHVDVEDR EEFKKTQDLL | ID |
| CRISPR- | RREVTGRLKE HENYTKIGKK DILDLLEKLP SISEEDYNAL ESFRNFYTYF | NO: |
| associated | TSYNKVRENL YSDEEKSSTV AYRLINENLP KFLDNIKSYA FVKAAGVLAD | 165) |
| protein Cpf1 | CIEEEEQDAL FMVETENMTL TQEGIDMYNY QIGKVNSAIN LYNQKNHKVE | |
| [Butyrivibrio sp. | EFKKIPKMKV LYKQILSDRE EVFIGEFKDD ETLLSSIGAY GNVLMTYLKS | |
| NC3005] | EKINIFFDAL RESEGKNVYV KNDLSKTTMS NIVFGSWSAF DELLNQEYDL | |
| | ANENKKKDDK YFEKRQKELK KNKSYTLEQM SNLSKEDISP IENYIERISE | |
| | DIEKICIYNG EFEKIVVNEH DSSRKLSKNI KAVKVIKDYL DSIKELEHDI | |
| | KLINGSGQEL EKNLVVYVGQ EEALEQLRPV DSLYNLTRNY LTKKPESTEK | |
| | VKLNENKSTL LNGWDKNKET DNLGILFFKD GKYYLGIMNT TANKAFVNPP | |
| | AAKTENVEKK VDYKLLPGSN KMLPKVFFAK SNIGYYNPST ELYSNYKKGT | |
| | HKKGPSFSID DCHNLIDFFK ESIKKHEDWS KFGFEFSDTA DYRDISEFYR | |
| | EVEKQGYKLT FTDIDESYIN DLIEKNELYL FQIYNKDESE YSKGKLNLHT | |
| | LYFMMLEDQR NLDNVVYKLN GEAEVFYRPA SIAENELVIH KAGEGIKNKN | |
| | PNRAKVKETS TFSYDIVKDK RYSKYKFTLH IPITMNFGVD EVRRENDVIN | |
| | NALRTDDNVN VIGIDRGERN LLYVVVINSE GKILEQISLN SIINKEYDIE | |
| | TNYHALLDER EDDRNKARKD WNTIENIKEL KTGYLSQVVN VVAKLVLKYN | |
| | AIICLEDLNF GEKRGRQKVE KQVYQKFEKM LIEKLNYLVI DKSREQVSPE | |
| | KMGGALNALQ LTSKFKSFAE LGKQSGIIYY VPAYLTSKID PTTGFVNLFY | |
| | IKYENIEKAK QFFDGEDFIR ENKKDDMFEF SFDYKSFTQK ACGIRSKWIV | |
| | YTNGERIIKY PNPEKNNLED EKVINVTDEI KGLFKQYRIP YENGEDIKEI | |
| | IISKAEADFY KRLFRLLHQT LQMRNSTSDG TRDYIISPVK NDRGEFFCSE | |
| | FSEGTMPKDA DANGAYNIAR KGLWVLEQIR QKDEGEKVNL SMTNAEWLKY | |
| | AQLHLL | |
| WP_027109509.1 | MENYYDSLTR QYPVTKTIRQ ELKPVGKTLE NIKNAEIIEA DKQKKEAYVK | (SEQ |
| type V CRISPR- | VKELMDEFHK SIIEKSLVGI KLDGLSEFEK LYKIKTKTDE DKNRISELFY | ID |
| associated | YMRKQIADAL KNSRDYGYVD NKDLIEKILP ERVKDENSLN ALSCFKGFTT | NO: |
| protein Cpf1 | YFTDYYKNRK NIYSDEEKHS TVGYRCINEN LLIFMSNIEV YQIYKKANIK | 166) |
| [Lachnospiraceae | NDNYDEETLD KTFMIESFNE CLTQSGVEAY NSVVASIKTA TNLYIQKNNK | |
| bacterium | EENFVRVPKM KVLFKQILSD RTSLEDGLII ESDDELLDKL CSFSAEVDKF | |
| NC2008] | LPINIDRYIK TLMDSNNGTG IYVKNDSSLT TLSNYLTDSW SSIRNAFNEN | |
| | YDAKYTGKVN DKYEEKREKA YKSNDSFELN YIQNLLGINV IDKYIERINE | |
| | DIKEICEAYK EMTKNCFEDH DKTKKLQKNI KAVASIKSYL DSLKNIERDI | |
| | KLINGTGLES RNEFFYGEQS TVLEEITKVD ELYNITRNYL TKKPESTEKM | |
| | KLNENNPQLL GGWDVNKERD CYGVILIKDN NYYLGIMDKS ANKSFLNIKE | |
| | SKNENAYKKV NCKLLPGPNK MFPKVFFAKS NIDYYDPTHE IKKLYDKGTF | |
| | KKGNSFNLED CHKLIDFYKE SIKKNDDWKN FNFNFSDTKD YEDISGFFRE | |
| | VEAQNYKITY TNVSCDFIES LVDEGKLYLF QIYNKDESEY ATGNLNLHTL | |
| | YLKMLEDERN LKDLCIKMNG EAEVFYRPAS ILDEDKVVHK ANQKITNKNT | |
| | NSKKKESIFS YDIVKDKRYT VDKFFIHLPI TLNYKEQNVS RENDYIREIL | |
| | KKSKNIRVIG IDRGERNLLY VVVCDSDGSI LYQRSINEIV SGSHKTDYHK | |
| | LLDNKEKERL SSRRDWKTIE NIKDLKAGYM SQVVNEIYNL ILKYNAIVVL | |
| | EDLNIGFKNG RKKVEKQVYQ NFEKALIDKL NYLCIDKTRE QLSPSSPGGV | |
| | LNAYQLTAKF ESFEKIGKQT GCIFYVPAYL TSQIDPTTGF VNLFYQKDTS | |
| | KQGLQLFFRK FKKINFDKVA SNFEFVEDYN DETNKAEGTK TNWTISTQGT | |
| | RIAKYRSDDA NGKWISRTVH PTDIIKEALN REKINYNDGH DLIDEIVSIE | |
| | KSAVLKEIYY GFKLTLQLRN STLANEEEQE DYIISPVKNS SGNYFDSRIT | |
| | SKELPCDADA NGAYNIARKG LWALEQIRNS ENVSKVKLAI SNKEWFEYTQ | |
| | NNIPSL | |
| WP_049895985.1 | METEILKYDF FEREGKYMYY DGLTKQYALS KTIRNELVPI GKTLDNIKKN | (SEQ |
| type V CRISPR- | RILEADIKRK SDYEHVKKLM DMYHKKIINE ALDNFKLSVL EDAADIYENK | ID |
| associated | QNDERDIDAF LKIQDKLRKE IVEQLKGHTD YSKVGNKDEL GLLKAASTEE | NO: |
| protein Cpf1 | DRILIESFDN FYTYFTSYNK VRSNLYSAED KSSTVAYRLI NENLPKFFDN | 167) |
| [Oribacterium | IKAYRTVRNA GVISGDMSIV EQDELFEVDT FNHTLTQYGI DTYNHMIGQL | |
| sp. NK2B42] | NSAINLYNQK MHGAGSFKKL PKMKELYKQL LTEREEEFIE EYTDDEVLIT | |
| WP_029202018 | SVHNYVSYLI DYLNSDKVES FEDTLRKSDG KEVFIKNDVS KTTMSNILED | |
| | NWSTIDDLIN HEYDSAPENV KKTKDDKYFE KRQKDLKKNK SYSLSKIAAL | |
| | CRDTTILEKY IRRLVDDIEK IYTSNNVFSD IVLSKHDRSK KLSKNTNAVQ | |
| | AIKNMLDSIK DFEHDVMLIN GSGQEIKKNL NVYSEQEALA GILRQVDHIY | |
| | NLTRNYLTKK PFSTEKIKLN FNRPTELDGW DKNKEEANLG ILLIKDNRYY | |
| | LGIMNTSSNK AFVNPPKAIS NDIYKKVDYK LLPGPNKMLP KVFFATKNIA | |
| | YYAPSEELLS KYRKGTHKKG DSFSIDDCRN LIDFFKSSIN KNTDWSTFGF | |
| | NFSDTNSYND ISDFYREVEK QGYKLSFTDI DACYIKDLVD NNELYLFQIY | |
| | NKDFSPYSKG KLNLHTLYFK MLFDQRNLDN VVYKLNGEAE VFYRPASIES | |
| | DEQIIHKSGQ NIKNKNQKRS NCKKTSTEDY DIVKDRRYCK DKFMLHLPIT | |
| | VNFGTNESGK FNELVNNAIR ADKDVNVIGI DRGERNLLYV VVVDPCGKII | |
| | EQISLNTIVD KEYDIETDYH QLLDEKEGSR DKARKDWNTI ENIKELKEGY | |
| | LSQVVNIIAK LVLKYDAIIC LEDLNFGFKR GRQKVEKQVY QKFEKMLIDK | |
| | MNYLVLDKSR KQESPQKPGG ALNALQLTSA FKSFKELGKQ TGIIYYVPAY | |
| | LTSKIDPTTG FANLFYIKYE SVDKARDFFS KEDFIRYNQM DNYFEFGEDY | |
| | KSFTERASGC KSKWIACTNG ERIVKYRNSD KNNSFDDKTV ILTDEYRSLE | |
| | DKYLQNYIDE DDLKDQILQI DSADFYKNLI KLFQLTLQMR NSSSDGKRDY | |
| | IISPVKNYRE EFFCSEFSDD TEPRDADANG AYNIARKGLW VIKQIRETKS | |
| | GTKINLAMSN SEWLEYAQCN LL | |
| WP_028248456.1 | MYYQNLTKMY PISKTLRNEL IPVGKTLENI RKNGILEADI QRKADYEHVK | (SEQ |
| type V CRISPR- | KLMDNYHKQL INEALQGVHL SDLSDAYDLY ENLSKEKNSV DAFSKCQDKL | ID |
| associated | RKEIVSLLKN HENFPKIGNK EIIKLLQSLY DNDTDYKALD SESNFYTYES | NO: |
| protein Cpf1 | SYNEVRKNLY SDEEKSSTVA YRLINENLPK FLDNIKAYAI AKKAGVRAEG | 168) |
| [Pseudo- | LSEEDQDCLF IIETFERTLT QDGIDNYNAA IGKLNTAINL FNQQNKKQEG | |
| butyrivibrio | FRKVPQMKCL YKQILSDREE AFIDEFSDDE DLITNIESFA ENMNVELNSE | |
| ruminis] | IITDEKIALV ESDGSLVYIK NDVSKTSFSN IVEGSWNAID EKLSDEYDLA | |
| | NSKKKKDEKY YEKRQKELKK NKSYDLETII GLEDDNSDVI GKYIEKLESD | |
| | ITAIAEAKND FDEIVLRKHD KNKSLRKNTN AVEAIKSYLD TVKDFERDIK | |
| | LINGSGQEVE KNLVVYAEQE NILAEIKNVD SLYNMSRNYL TQKPESTEKF | |
| | KLNFNRATLL NGWDKNKETD NLGILFEKDG MYYLGIMNTK ANKIFVNIPK | |
| | ATSNDVYHKV NYKLLPGPNK MLPKVFFAQS NLDYYKPSEE LLAKYKAGTH | |
| | KKGDNFSLED CHALIDFFKA SIEKHPDWSS FGFEFSETCT YEDLSGFYRE | |
| | VEKQGYKITY TDVDADYITS LVERDELYLF QIYNKDESPY SKGNLNLHTI | |
| | YLQMLFDQRN LNNVVYKLNG EAEVFYRPAS INDEEVIIHK AGEEIKNKNS | |
| | KRAVDKPTSK FGYDIIKDRR YSKDKFMLHI PVTMNFGVDE TRRENDVVND | |
| | ALRNDEKVRV IGIDRGERNL LYVVVVDTDG TILEQISLNS IINNEYSIET | |
| | DYHKLLDEKE GDRDRARKNW TTIENIKELK EGYLSQVVNV IAKLVLKYNA | |
| | IICLEDLNFG FKRGRQKVEK QVYQKFEKML IDKLNYLVID KSRKQDKPEE | |
| | FGGALNALQL TSKFTSFKDM GKQTGIIYYV PAYLTSKIDP TTGFANLFYV | |
| | KYENVEKAKE FFSREDSISY NNESGYFEFA FDYKKETDRA CGARSQWTVC | |
| | TYGERIIKFR NTEKNNSEDD KTIVLSEEFK ELFSIYGISY EDGAELKNKI | |
| | MSVDEADFFR SLTRLFQQTM QMRNSSNDVT RDYIISPIMN DRGEFENSEA | |
| | CDASKPKDAD ANGAFNIARK GLWVLEQIRN TPSGDKLNLA MSNAEWLEYA | |
| | QRNQI | |
| WP_028830240 | MENFKNLYPI NKTLRFELRP YGKTLENFKK SGLLEKDAFK ANSRRSMQAI | (SEQ |
| type V CRISPR- | IDEKFKETIE ERLKYTEFSE CDLGNMTSKD KKITDKAATN LKKQVILSED | ID |
| associated | DEIFNNYLKP DKNIDALFKN DPSNPVISTF KGFTTYFVNF FEIRKHIFKG | NO: |
| protein Cpf1 | ESSGSMAYRI IDENLTTYLN NIEKIKKLPE ELKSQLEGID QIDKLNNYNE | 169) |
| [Proteocatella | FITQSGITHY NEIIGGISKS ENVKIQGINE GINLYCQKNK VKLPRLTPLY | |
| sphenisci] | KMILSDRVSN SFVLDTIEND TELIEMISDL INKTEISQDV IMSDIQNIFI | |
| | KYKQLGNLPG ISYSSIVNAI CSDYDNNFGD GKRKKSYEND RKKHLETNVY | |
| | SINYISELLT DTDVSSNIKM RYKELEQNYQ VCKENFNATN WMNIKNIKQS | |
| | EKTNLIKDLL DILKSIQRFY DLFDIVDEDK NPSAEFYTWL SKNAEKLDFE | |
| | FNSVYNKSRN YLTRKQYSDK KIKLNEDSPT LAKGWDANKE IDNSTIIMRK | |
| | FNNDRGDYDY FLGIWNKSTP ANEKIIPLED NGLFEKMQYK LYPDPSKMLP | |
| | KQFLSKIWKA KHPTTPEFDK KYKEGRHKKG PDFEKEFLHE LIDCFKHGLV | |
| | NHDEKYQDVF GENLRNTEDY NSYTEFLEDV ERCNYNLSEN KIADTSNLIN | |
| | DGKLYVFQIW SKDFSIDSKG TKNINTIYFE SLFSEENMIE KMFKLSGEAE | |
| | IFYRPASLNY CEDIIKKGHH HAELKDKEDY PIIKDKRYSQ DKFFFHVPMV | |
| | INYKSEKLNS KSLNNRTNEN LGQFTHIIGI DRGERHLIYL TVVDVSTGEI | |
| | VEQKHLDEII NTDTKGVEHK THYLNKLEEK SKTRDNERKS WEAIETIKEL | |
| | KEGYISHVIN EIQKLQEKYN ALIVMENLNY GFKNSRIKVE KQVYQKFETA | |
| | LIKKENYIID KKDPETYIHG YQLTNPITTL DKIGNQSGIV LYIPAWNTSK | |
| | IDPVTGFVNL LYADDLKYKN QEQAKSFIQK IDNIYFENGE FKFDIDESKW | |
| | NNRYSISKTK WTLTSYGTRI QTERNPQKNN KWDSAEYDLT EEFKLILNID | |
| | GTLKSQDVET YKKEMSLFKL MLQLRNSVTG TDIDYMISPV TDKTGTHEDS | |
| | RENIKNLPAD ADANGAYNIA RKGIMAIENI MNGISDPLKI SNEDYLKYIQ | |
| | NQQE | |
| WP_084502895.1 | MIILYISTSN MNMEGVFMEN FKNLYPINKT LRFELRPYGK TLENFKKSGL | (SEQ |
| type V CRISPR- | LEKDAFKANS RRSMQAIIDE KEKETIEERL KYTEFSECDL GNMTSKDKKI | ID |
| associated | TDKAATNLKK QVILSEDDEI ENNYLKPDKN IDALFKNDPS NPVISTEKGF | NO: |
| protein Cpf1 | TTYFVNFFEI RKHIFKGESS GSMAYRIIDE NLTTYLNNIE KIKKLPEELK | 170) |
| [Proteocatella | SQLEGIDQID KLNNYNEFIT QSGITHYNEI IGGISKSENV KIQGINEGIN | |
| sphenisci] | LYCQKNKVKL PRLTPLYKMI LSDRVSNSFV LDTIENDTEL IEMISDLINK | |
| | TEISQDVIMS DIQNIFIKYK QLGNLPGISY SSIVNAICSD YDNNFGDGKR | |
| | KKSYENDRKK HLETNVYSIN YISELLTDTD VSSNIKMRYK ELEQNYQVCK | |
| | ENFNATNWMN IKNIKQSEKT NLIKDLLDIL KSIQRFYDLF DIVDEDKNPS | |
| | AEFYTWLSKN AEKLDFEFNS VYNKSRNYLT RKQYSDKKIK LNFDSPTLAK | |
| | GWDANKEIDN STIIMRKENN DRGDYDYFLG IWNKSTPANE KIIPLEDNGL | |
| | FEKMQYKLYP DPSKMLPKQF LSKIWKAKHP TTPEFDKKYK EGRHKKGPDF | |
| | EKEFLHELID CFKHGLVNHD EKYQDVEGEN LRNTEDYNSY TEFLEDVERC | |
| | NYNLSENKIA DTSNLINDGK LYVFQIWSKD FSIDSKGTKN LNTIYFESLF | |
| | SEENMIEKMF KLSGEAEIFY RPASLNYCED IIKKGHHHAE LKDKFDYPII | |
| | KDKRYSQDKF FFHVPMVINY KSEKLNSKSL NNRTNENLGQ FTHIIGIDRG | |
| | ERHLIYLTVV DVSTGEIVEQ KHLDEIINTD TKGVEHKTHY LNKLEEKSKT | |
| | RDNERKSWEA IETIKELKEG YISHVINEIQ KLQEKYNALI VMENLNYGFK | |
| | NSRIKVEKQV YQKFETALIK KENYIIDKKD PETYIHGYQL TNPITTLDKI | |
| | GNQSGIVLYI PAWNTSKIDP VTGFVNLLYA DDLKYKNQEQ AKSFIQKIDN | |
| | IYFENGEFKF DIDFSKWNNR YSISKTKWTL TSYGTRIQTF RNPQKNNKWD | |
| | SAEYDLTEEF KLILNIDGTL KSQDVETYKK FMSLFKLMLQ LRNSVTGTDI | |
| | DYMISPVTDK TGTHEDSREN IKNLPADADA NGAYNIARKG IMAIENIMNG | |
| | ISDPLKISNE DYLKYIQNQQ E | |
| WP_055225123.1 | MNNGTNNFQN FIGISSLQKT LRNALIPTET TQQFIVKNGI IKEDELRGEN | (SEQ |
| Eubacterium | RQILKDIMDD YYRGFISETL SSIDDIDWTS LFEKMEIQLK NGDNKDTLIK | ID |
| rectale | EQTEYRKAIH KKFANDDREK NMESAKLISD ILPEFVIHNN NYSASEKEEK | NO: |
| | TQVIKLESRF ATSFKDYFKN RANCESADDI SSSSCHRIVN DNAEIFFSNA | 171) |
| | LVYRRIVKSL SNDDINKISG DMKDSLKEMS LEEIYSYEKY GEFITQEGIS | |
| | FYNDICGKVN SEMNLYCQKN KENKNLYKLQ KLHKQILCIA DTSYEVPYKF | |
| | ESDEEVYQSV NGELDNISSK HIVERLRKIG DNYNGYNLDK IYIVSKFYES | |
| | VSQKTYRDWE TINTALEIHY NNILPGNGKS KADKVKKAVK NDLQKSITEI | |
| | NELVSNYKLC SDDNIKAETY IHEISHILNN FEAQELKYNP EIHLVESELK | |
| | ASELKNVLDV IMNAFHWCSV FMTEELVDKD NNFYAELEEI YDEIYPVISL | |
| | YNLVRNYVTQ KPYSTKKIKL NFGIPTLADG WSKSKEYSNN AIILMRDNLY | |
| | YLGIFNAKNK PDKKIIEGNT SENKGDYKKM IYNLLPGPNK MIPKVFLSSK | |
| | TGVETYKPSA YILEGYKQNK HIKSSKDFDI TECHDLIDYF KNCIAIHPEW | |
| | KNFGFDFSDT STYEDISGFY REVELQGYKI DWTYISEKDI DLLQEKGQLY | |
| | LFQIYNKDFS KKSTGNDNLH TMYLKNLFSE ENLKDIVLKL NGEAEIFFRK | |
| | SSIKNPIIHK KGSILVNRTY EAEEKDQFGN IQIVRKNIPE NIYQELYKYF | |
| | NDKSDKELSD EAAKLKNVVG HHEAATNIVK DYRYTYDKYF LHMPITINFK | |
| | ANKTGFINDR ILQYIAKEKD LHVIGIDRGE RNLIYVSVID TCGNIVEQKS | |
| | FNIVNGYDYQ IKLKQQEGAR QIARKEWKEI GKIKEIKEGY LSLVIHEISK | |
| | MVIKYNAIIA MEDLSYGFKK GREKVERQVY QKFETMLINK LNYLVFKDIS | |
| | ITENGGLLKG YQLTYIPDKL KNVGHQCGCI FYVPAAYTSK IDPTTGFVNI | |
| | FKFKDLTVDA KREFIKKFDS IRYDSEKNLF CFTFDYNNFI TQNTVMSKSS | |
| | WSVYTYGVRI KRRFVNGRES NESDTIDITK DMEKTLEMTD INWRDGHDLR | |
| | QDIIDYEIVQ HIFEIFRLTV QMRNSLSELE DRDYDRLISP VLNENNIFYD | |
| | SAKAGDALPK DADANGAYCI ALKGLYEIKQ ITENWKEDGK FSRDKLKISN | |
| | KDWFDFIQNK RYL | |
| WP_055237260.1 | MNNGTNNFQN FIGISSLQKT LRNALIPTET TQQFIVKNGI IKEDELRGEN | (SEQ |
| Eubacterium | RQILKDIMDD YYRGFISETL SSIDDIDWTS LFEKMEIQLK NGDNKDTLIK | ID |
| rectale | EQAEKRKAIY KKFADDDRFK NMFSAKLISD ILPEFVIHNN NYSASEKEEK | NO: |
| | TQVIKLESRF ATSFKDYFKN RANCESADDI SSSSCHRIVN DNAEIFFSNA | 172) |
| | LVYRRIVKNL SNDDINKISG DMKDSLKEMS LDEIYSYEKY GEFITQEGIS | |
| | FYNDICGKVN SFMNLYCQKN KENKNLYKLR KLHKQILCIA DTSYEVPYKF | |
| | ESDEEVYQSV NGELDNISSK HIVERLRKIG DNYNGYNLDK IYIVSREYES | |
| | VSQKTYRDWE TINTALEIHY NNILPGNGKS KADKVKKAVK NDLQKSITEI | |
| | NELVSNYKLC PDDNIKAETY IHEISHILNN FEAQELKYNP EIHLVESELK | |
| | ASELKNVLDV IMNAFHWCSV FMTEELVDKD NNFYAELEEI YDEIYPVISL | |
| | YNLVRNYVTQ KPYSTKKIKL NEGIPTLADG WSKSKEYSNN AIILMRDNLY | |
| | YLGIFNAKNK PDKKIIEGNT SENKGDYKKM IYNLLPGPNK MIPKVFLSSK | |
| | TGVETYKPSA YILEGYKQNK HLKSSKDEDI TFCRDLIDYF KNCIAIHPEW | |
| | KNFGFDESDT STYEDISGFY REVELQGYKI DWTYISEKDI DLLQEKGQLY | |
| | LFQIYNKDES KKSTGNDNLH TMYLKNLESE ENLKDIVLKL NGEAEIFFRK | |
| | SSIKNPIIHK KGSILVNRTY EAEEKDQFGN IQIVRKTIPE NIYQELYKYF | |
| | NDKSDKELSD EAAKLKNVVG HHEAATNIVK DYRYTYDKYF LHMPITINFK | |
| | ANKTSFINDR ILQYIAKEND LHVIGIDRGE RNLIYVSVID TCGNIVEQKS | |
| | FNIVNGYDYQ IKLKQQEGAR QIARKEWKEI GKIKEIKEGY LSLVIHEISK | |
| | MVIKYNAIIA MEDLSYGFKK GRFKVERQVY QKFETMLINK LNYLVEKDIS | |
| | ITENGGLLKG YQLTYIPEKL KNVGHQCGCI FYVPAAYTSK IDPTTGFANI | |
| | FKFKDLTVDA KREFIKKFDS IRYDSEKNLF CFTEDYNNFI TQNTVMSKSS | |
| | WSVYTYGVRI KRRFVNGRES NESDTIDITK DMEKTLEMTD INWRDGHDLR | |
| | QDIIDYEIVQ HIFEIFKLTV QMRNSLSELE DRDYDRLISP VLNENNIFYD | |
| | SAKAGDALPK DADANGAYCI ALKGLYEIKQ ITENWKEDGK FSRDKLKISN | |
| | KDWFDFIQNK RYL | |
| WP_055272206.1 | MNNGTNNFQN FIGISSLQKT LRNALTPTET TQQFIVKNGI IKEDELRGEN | (SEQ |
| Eubacterium | RQILKDIMDD YYRGFISETL SSIDDIDWTS LFEKMEIQLK NGDNKDTLIK | ID |
| rectale | EQAEKRKAIY KKFADDDREK NMFSAKLISD ILPEFVIHNN NYSASEKEEK | NO: |
| | TQVIKLESRF ATSFKDYFKN RANCESADDI SSSSCHRIVN DNAEIFFSNA | 173) |
| | LVYRRIVKNL SNDDINKISG DMKDSLKKMS LEKIYSYEKY GEFITQEGIS | |
| | FYNDICGKVN SEMNLYCQKN KENKNLYKLR KLHKQILCIA DTSYEVPYKE | |
| | ESDEEVYQSV NGELDNISSK HIVERLRKIG DNYNGYNLDK IYIVSKFYES | |
| | VSQKTYRDWE TINTALEIHY NNILPGNGKS KADKVKKAVK NDLQKSITEI | |
| | NELVSNYKLC PDDNIKAETY IHEISHILNN FEAQELKYNP EIHLVESELK | |
| | ASELKNVLDV IMNAFHWCSV FMTEELVDKD NNFYAELEEI YDEIYPVISL | |
| | YNLVRNYVTQ KPYSTKKIKL NFGIPTLADG WSKSKEYSNN AIILMRDNLY | |
| | YLGIFNAKNK PEKKIIEGNT SENKGDYKKM IYNLLPGPNK MIPKVFLSSK | |
| | TGVETYKPSA YILEGYKQNK HLKSSKDEDI TFCRDLIDYF KNCIAIHPEW | |
| | KNFGFDESDT STYEDISGFY REVELQGYKI DWTYISEKDI DLLQEKGQLY | |
| | LFQIYNKDFS KKSTGNDNLH TMYLKNLFSE ENLKDVVLKL NGEAEIFFRK | |
| | SSIKNPIIHK KGSILVNRTY EAEEKDQFGN IQIVRKTIPE NIYQELYKYF | |
| | NDKSDKELSD EAAKLKNAVG HHEAATNIVK DYRYTYDKYF LHMPITINEK | |
| | ANKTSFINDR ILQYIAKEKD LHVIGIDRGE RNLIYVSVID TCGNIVEQKS | |
| | FNIVNGYDYQ IKLKQQEGAR QIARKEWKEI GKIKEIKEGY LSLVIHEISK | |
| | MVIKYNAIIA MEDLSYGEKK GREKVERQVY QKFETMLINK LNYLVEKDIS | |
| | ITENGGLLKG YQLTYIPEKL KNVGHQCGCI FYVPAAYTSK IDPTTGFVNI | |
| | FKFKDLTVDA KREFIKKFDS IRYDSDKNLF CFTEDYNNFI TQNTVMSKSS | |
| | WSVYTYGVRI KRRFVNGRES NESDTIDITK DMEKTLEMTD INWRDGHDLR | |
| | QDIIDYEIVQ HIFEIFKLTV QMRNSLSELE DRNYDRLISP VLNENNIFYD | |
| | SAKAGDALPK DADANGAYCI ALKGLYEIKQ ITENWKEDGK FSRDKLKISN | |
| | KDWEDFIQNK RYL | |
| OLA16049.1 | MNNGTNNFQN FIGISSLQKT LRNALIPTET TQQFIVKNGI IKEDELRGKN | (SEQ |
| Eubacterium sp. | RQILKDIMDD YYRGFISETL SSIDDIDWTS LFEKMEIQLK NGDNKDTLIK | ID |
| 41 20 | EQAEKRKAIY KKFADDDREK NMESAKLISD ILPEFVIHNN NYSASEKKEK | NO: |
| | TQVIKLESRF ATSFKDYFKN RANCESADDI SSSSCHRIVN DNAEIFFSNA | 174) |
| | LVYRRIVKNL SNDDINKISG DMKDSLKEMS LEEIYSYEKY GEFITQEGIS | |
| | FYNDICGKVN SEMNLYCQKN KENKNLYKLR KLHKQILCIA DTSYEVPYKE | |
| | ESDEEVYQSV NGELDNISSK HIVERLRKIG DNYNDYNLDK IYIVSKFYES | |
| | VSQKTYRDWE TINTALEIHY NNILPGNGKS KADKVKKAVK NDLQKSITEI | |
| | NELVSNYKLC SDDNIKAETY IHEISHILNN FEAHELKYNP EIHLVESELK | |
| | ASELKNVLDI IMNAFHWCSV FMTEELVDKD NNFYAELEEI YDEIYPVISL | |
| | YNLVRNYVTQ KPYSTKKIKL NEGIPTLADG WSKSKEYSNN AIILMRDNLY | |
| | YLGIFNAKNK PDKKIIEGNT SENKGDYKKM IYNLLPGPNK MIPKVFLSSK | |
| | TGVETYKPSA YILEGYKQNK HLKSSKDEDI TECHDLIDYF KNCIAIHPEW | |
| | KNFGFDFSDT SAYEDISGFY REVELQGYKI DWTYISEKDI DLLQEKGQLY | |
| | LFQIYNKDFS KKSTGNDNLH TMYLKNLFSE ENLKDIVLKL NGEAEIFFRK | |
| | SSIKNPIIHK KGSILVNRTY EAEEKDQFGN IQIVRKTIPE NIYQELYKYF | |
| | NDKSDKELSD EAAKLKNVVG HHEAATNIVK DYRYTYDKYF LHMPITINFK | |
| | ANKTSFINDR ILQYIAKEKD LHVIGIDRGE RNLIYVSVID TCGNIVEQKS | |
| | FNIVNGYDYQ IKLKQQEGAR QIARKEWKEI GKIKEIKEGY LSLVIHEISK | |
| | MVIKYNAIIA MEDLSYGFKK GRFKVERQVY QKFETMLINK LNYLVFKDIS | |
| | ITENGGLLKG YQLTYIPDKL KNVGHQCGCI FYVPAAYTSK IDPTTGFVNI | |
| | FKFKDLTVDA KREFIKKFDS IRYDSEKNLF CFTEDYNNFI TQNTVMSKSS | |
| | WSVYTYGVRI KRRFVNGRFS NESDTIDITK DMEKTLEMTD INWRDGHDLR | |
| | QDIIDYEIVQ HIFEIFKLTV QMRNSLSELE DRDYDRLISP VLNENNIFYD | |
| | SAKAGYALPK DADANGAYCI ALKGLYEIKQ ITENWKEDGK FSRDKLKISN | |
| | KDWFDFIQNK RYL | |

TABLE 6
Cas12b (C2c1) orthologs

| | | |
|---|---|---|
| Alicyclobacillus | MVAVKSIKVK LMLGHLPEIR EGLWHLHEAV NLGVRYYTEW LALLRQGNLY | (SEQ |
| macrosporangiidus | RRGKDGAQEC YMTAEQCRQE LLVRLRDRQK RNGHTGDPGT DEELLGVARR | ID |
| strain DSM | LYELLVPQSV GKKGQAQMLA SGELSPLADP KSEGGKGTSK SGRKPAWMGM | NQ: |
| 17980 | KEAGDSRWVE AKARYEANKA KDPTKQVIAS LEMYGLRPLF DVFTETYKTI | 175) |
| WP_074948407.1 | RWMPLGKHQG VRAWDRDMFQ QSLERLMSWE SWNERVGAEF ARLVDRRDRE | |
| | REKHETGQEH LVALAQRLEQ EMKEASPGFE SKSSQAHRIT KRALRGADGI | |
| | IDDWLKLSEG EPVDREDEIL RKRQAQNPRR FGSHDLFLKL AEPVFQPLWR | |
| | EDPSELSRWA SYNEVLNKLE DAKQFATFTL PSPCSNPVWA RFENAEGTNI | |
| | FKYDFLFDHF GKGRHGVRFQ RMIVMRDGVP TEVEGIVVPI APSRQLDALA | |
| | PNDAASPIDV FVGDPAAPGA FRGQFGGAKI QYRRSALVRK GRREEKAYLC | |
| | GFRLPSQRRT GTPADDAGEV FLNLSLRVES QSEQAGRRNP PYAAVFHISD | |
| | QTRRVIVRYG EIERYLAEHP DTGIPGSRGL TSGLRVMSVD LGLRTSAAIS | |
| | VERVAHRDEL TPDAHGRQPF FFPIHGMDHL VALHERSHLI RLPGETESKK | |
| | VRSIREQRLD RLNRLRSQMA SLRLLVRTGV LDEQKRDRNW ERLQSSMERG | |
| | GERMPSDWWD LFQAQVRYLA QHRDASGEAW GRMVQAAVRT LWRQLAKQVR | |
| | DWRKEVRRNA DKVKIRGIAR DVPGGHSLAQ LDYLERQYRF LRSWSAFSVQ | |
| | AGQVVRAERD SRFAVALREH IDNGKKDRLK KLADRILMEA LGYVYVTDGR | |
| | RAGQWQAVYP PCQLVLLEEL SEYRESNDRP PSENSQLMVW SHRGVLEELI | |
| | HQAQVHDVLV GTIPAAFSSR FDARTGAPGI RCRRVPSIPL KDAPSIPIWL | |
| | SHYLKQTERD AAALRPGELI PTGDGEFLVT PAGRGASGVR VVHADINAAH | |
| | NLQRRLWENF DLSDIRVRCD RREGKDGTVV LIPRLTNQRV KERYSGVIFT | |
| | SEDGVSFTVG DAKTRRRSSA SQGEGDDLSD EEQELLAEAD DARERSVVLE | |
| | RDPSGFVNGG RWTAQRAFWG MVHNRIETLL AERFSVSGAA EKVRG | |
| Bacillus hisashii | MATRSFILKI EPNEEVKKGL WKTHEVLNHG IAYYMNILKL IRQEAIYEHH | (SEQ |
| strain C4 | EQDPKNPKKV SKAEIQAELW DFVLKMQKCN SFTHEVDKDE VENILRELYE | ID |
| WP_095142515.1 | ELVPSSVEKK GEANQLSNKF LYPLVDPNSQ SGKGTASSGR KPRWYNLKIA | NO: |
| | GDPSWEEEKK KWEEDKKKDP LAKILGKLAE YGLIPLFIPY TDSNEPIVKE | 176) |
| | IKWMEKSRNQ SVRRLDKDMF IQALERFLSW ESWNLKVKEE YEKVEKEYKT | |
| | LEERIKEDIQ ALKALEQYEK ERQEQLLRDT LNTNEYRLSK RGLRGWREII | |
| | QKWLKMDENE PSEKYLEVEK DYQRKHPREA GDYSVYEFLS KKENHFIWRN | |
| | HPEYPYLYAT FCEIDKKKKD AKQQATFTLA DPINHPLWVR FEERSGSNLN | |
| | KYRILTEQLH TEKLKKKLTV QLDRLIYPTE SGGWEEKGKV DIVLLPSRQF | |
| | YNQIFLDIEE KGKHAFTYKD ESIKFPLKGT LGGARVQFDR DHLRRYPHKV | |
| | ESGNVGRIYF NMTVNIEPTE SPVSKSLKIH RDDEPKVVNF KPKELTEWIK | |
| | DSKGKKLKSG IESLEIGLRV MSIDLGQRQA AAASIFEVVD QKPDIEGKLF | |
| | FPIKGTELYA VHRASENIKL PGETLVKSRE VLRKAREDNL KLMNQKLNFL | |
| | RNVLHFQQFE DITEREKRVT KWISRQENSD VPLVYQDELI QIRELMYKPY | |
| | KDWVAFLKQL HKRLEVEIGK EVKHWRKSLS DGRKGLYGIS LKNIDEIDRT | |
| | RKFLLRWSLR PTEPGEVRRL EPGQRFAIDQ LNHLNALKED RLKKMANTII | |
| | MHALGYCYDV RKKKWQAKNP ACQIILFEDL SNYNPYEERS RFENSKLMKW | |
| | SRREIPRQVA LQGEIYGLQV GEVGAQFSSR FHAKTGSPGI RCSVVTKEKL | |
| | QDNRFFKNLQ REGRLTLDKI AVLKEGDLYP DKGGEKFISL SKDRKCVTTH | |
| | ADINAAQNLQ KRFWTRTHGF YKVYCKAYQV DGQTVYIPES KDQKQKIIEE | |
| | FGEGYFILKD GVYEWVNAGK LKIKKGSSKQ SSSELVDSDI LKDSEDLASE | |
| | LKGEKLMLYR DPSGNVFPSD KWMAAGVFFG KLERILISKL TNQYSISTIE | |
| | DDSSKQSM | |
| Candidatus | MPRDDLDLLT NLNSTAKGIR ERGKTKEGTD KKKSGRKSSW PMDKAAWETA | (SEQ |
| Lindowbacteria | KTSDSSAHFL EKLKQHPDLK DAFGNLSSGG SKKLEYYKKL AGSAPWKESQ | ID |
| bacterium | SVILEKAARW KEAKQEREEK EQDSSEHGSK AAYRRLEDAG CLPMPEFAKY | NO: |
| RIFCSPLOWO2 | IDENQIEFGD LKLSDCGAEW KRGMWNQAGQ RVRSHMGWQR RREKENAVYS | 177 |
| OGH55994.1 | LRKELFEKGG AIRRKKSEEL TPEDILPGKA APDQNDWQER PAYGNQMWFI | |
| | GLRSYEENEM AKYAEEAGMG SRSAPRIRRG TIKGWSKLRE RWLQILKRNP | |
| | QATRDDLIGE LNALRSQDPR AYGDARLEDW LSKTDQRFLW DGEDADGKIL | |
| | CGRDDRDCVS AFVAYNEEFA DEPSSITLTE TDERLHPVWP FFGESSAVPY | |
| | EIEYDLETAC PTAIRLPLLV GKENGGYAER QGTRIPLAEY ADLASSFQLP | |
| | TPVRLDVLVE IREVTRAGRK VTCPFSYFKQ NGVWYVREGE IPSGESIQIK | |
| | QTDRKIENGK IFISSKLRMA YRDDLMVSPA TGDEGSIKIL WERIELASHV | |
| | DQKKLPETAP ARSRVFVSFS CNVVERAPRK QLTRKPDAVV VTIPSGVDQG | |
| | LVVVSTDVRT GKSKSSSAPP LPPGSRLWPA DAVHGDPPLR ILSVDLGHRH | |
| | SAYAVWELGL QQKSWRAGVL KGSTQTPVYA DCTGTGLLCL PGDGEDTPAE | |
| | EESLRLRSRQ IRRRLNLQNS ILRVSRLLSL DKFEKTIFEQ SDVRDRPNKK | |
| | GLRIRRRCRT EKTPLSEAEV RKNCDKAAEI LIRWADTDAM AKSLAATGNA | |
| | DISFWKYMAV KNPPLSAVVD VAPSTIVPDD GPDRETLKKK RQEEEEKFAS | |
| | SIYENRVKLA GALCSGYDAD HRRPATGGLW HDLDRTLIRE ISYGDRGQKG | |
| | NPRKLNNEGI LRLLRRPPRA RPDWREFHRT LNDANRIPKG RTLRGGLSMG | |
| | RLNFLKEVGD FVKKWSCRPR WPGDRRHIPP GQLFDRQDAE HLEHLRDDRI | |
| | KRLAHLIVAQ ALGFEPDIRR GLWKYVDGST GEILWQHPET RRFFAEGAAG | |
| | ELREVSRPAE IDDDAAARPH TVSAPAHIVV FENLIRYRFQ SDRPKTENAG | |
| | LMQWAHRQIV HFTKQVASLY GLKVAMVYAA FSSKFCSRCG SPGARVSRED | |
| | PAWRNQEWFK RRTSNPRSKV DHSLKRASED PTADETRPWV LIEGGKEFVC | |
| | ANAKCSAHDE PLNADENAAA NIGLRFLRGV EDFRTKVNPA GALKGKLRFE | |
| | TGIHSFRPPV SGSPEWSPMA EPAQKKKIGA AAPGADVDEA GDADESGVVV | |
| | LFRDPSGAFR NKQYWYEGKI FWSNVMMAVE AKIAGASVGA KPVAASWGQA | |
| | QPQSGPGLAK PGGD | |
| Elusimicrobia | MNRIYQGRVT KVEVPDGKDE KGNIKWKKLE NWSDILWQHH MLFQDAVNYY | (SEQ |
| bacterium | TLALAAISGS AVGSDEKSII LREWAVQVQN IWEKAKKKAT VFEGPQKRLT | ID |
| RIFOXYA12 | SILGLEQNAS FDIAAKHILR TSEAKPEQRA SALIRLLEEI DKKNHNVVCG | NO: |
| OGS02326.1 | ERLPFFCPRN IQSKRSPTSK AVSSVQEQKR QEEVRRFHNM QPEEVVKNAV | 178) |
| | TLDISLEKSS PKIVELEDPK KARAELLKQF DNACKKHKEL VGIKKAFTES | |
| | IDKHGSSLKV PAPGSKPSGL YPSAIVFKYF PVDITKTVEL KATEKLAMGK | |
| | DREVTNDPIA DARVNDKPHE DYFTNIALIR EKEKNRAAWF EFDLAAFIEA | |
| | IMSPHRFYQD TQKRKEAARK LEEKIKAIEG KGGQFKESDS EDDDVDSLPG | |
| | FEGDTRIDLL RKLVTDTLGW LGESETPDNN EGKKTEYSIS ERTLRIFPDI | |
| | QKQWSELAEK GETTEGKLLE VLKHEQTEHQ SDFGSATLYQ HLAKPEFHPI | |
| | WLKSGTEEWH AENPLKAWLN YKELQYELTD KKRPIHFTPA HPVYSPRYED | |
| | FPKKSETEEK EVSKNTHSLT TSLASEHIKN SLQFTAGLIR KTNVGKKAIK | |
| | ARFSYSAPRL RRDCLRSENN ENLYKAPWLQ PMMRALGIDE EKADRQNFAN | |
| | TRITLMAKGL DDIQLGFPVE ANSQELQKEV SNGISWKGQF NWGGIASLSA | |
| | LRWPHEKKPK NPPEQPWWGI DSFSCLAVDL GQRYAGAFAR LDVSTIEKKG | |
| | KSRFIGEACD KKWYAKVSRM GLLRLPGEDV KVWRDASKID KENGFAFRKE | |
| | LFGEKGRSAT PLEAEETAEL IKLFGANEKD VMPDNWSKEL SFPEQNDKLL | |
| | IVARRAQAAV SRLHRWAWFF DEAKRSDDAI REILESDDTD LKQKVNKNEI | |
| | EKVKETIISL LKVKQELLPT LLTRLANRVL PLRGRSWEWK KHHQKNDGFI | |
| | LDQTGKAMPN VLIRGQRGLS MDRIEQITEL RKRFQALNQS LRRQIGKKAP | |
| | AKRDDSIPDC CPDLLEKLDH MKEQRVNQTA HMILAEALGL KLAEPPKDKK | |
| | ELNETCDMHG AYAKVDNPVS FIVIEDLSRY RSSQGRSPRE NSRLMKWCHR | |
| | AVRDKLKEMC EVFFPLCERR KAGSAWVSLP PLLETPAAYS SRFCSRSGVA | |
| | GFRAVEVIPG FELKYPWSWL KDKKDKAGNL AKEALNIRTV SEQLKAFNQD | |
| | KPEKPRTLLV PIAGGPIFVP ISEVGLSSFG LKPQVVQADI NAAINLGLRA | |
| | ISDPRIWEIH PRLRTEKRDG RLFAREKRKY GEEKVEVQPS KNEKAKKVKD | |
| | DRKPNYFADF SGKVDWGFGN IKNESGLTLV SGKALWWTIN QLQWERCEDI | |
| | NKRHIEDWSN KQKQ | |
| Omnitrophica | MNRIYQGRVT KVEKLKNGKS PDDREELKDW QTALWRHHEL FQDAVSYYTL | (SEQ |
| WOR_2 | ALAAMAEGLP DKHPINVLRK RMEEAWEEFP RKTVTPAKNL RDSVRPWLGL | ID |
| bacterium | SESASFGDAL KKILPPAPEN KEVRALAVAL LAEKARTLKP QKTSASYWGR | NO: |
| RIFCSPHIGHO | FCDDLKKKPN WDYSEEELAR KTGSGDWVAG LWSEDALNKI DELAKSLKLS | 179) |
| 2 | SLVKCVPDGQ INPEGARNLV KEALDHLEGV SNGTKKEKND PGPAKKTNNW | |
| OGX36711.1 | LRQHASDVRN FIHKNKNQFS SLPNGRLITE RARGGGININ KTYAGVLFKA | |
| | FPCPFTEDYV RAAVPEPKVK KVDQEKKSEQ SATWTELEKR ILRIGDDPIE | |
| | LARKNNKPIF KAFTALEKWS DQNSKSCWSD FDKCAFEEAL KTLNQFNQKT | |
| | EEREKRRSEA EAELKYMMDE NPEWKPKKET EGDDVREVPI LKGDPRYEKL | |
| | VKLFGDLDEE GSEHATGKIY GPSRASLRGF GKLRNEWVDL FTKANDNPRE | |
| | QDLQKAVTGF QREHKLDMGY TAFFLKLCER DYWDIWRDDT EVEVKKIREK | |
| | RWVKSVVYAA ADTRELAEEL ERLQEPVRYT PAEPQFSRRL FMFSDIKGKQ | |
| | GAKHIREGLV EVSLAVKDQS GKYGTCRVRL HYSAPRLIRD HLSDGSSSMW | |
| | LQPMMAALGL SSDARGCFTR DSKGNVKEPA VALMSDFVGR KRELRMLLNE | |
| | PVDLDISKLE ENIGKKARWE KQMNTAYEKN KLKQRFHLIW PGMELKETQE | |
| | PGQFWWDNPT IQKEGMYCLA IDLSQRRAAD YALLHAGVNR DSKTEVELGQ | |
| | AGGQSWFTKL CAAGSLRLPG EDTEVIREGK RQIELSGKKG RNATQSEYDQ | |
| | AIALAKQLLH NENSAELESA ARDWLGDNAK RESFPEQNDK LIDLYYGALS | |
| | RYKTWLRWSW RLTEQHKELW DKTLDEIRKV PYFASWGELA GNGTNEATVQ | |
| | QLQKLIADAA VDLRNFLEKA LLHIAYRALP LRENTWRWIE NGKDGKGKPL | |
| | HLLVSDGQSP AEIPWLRGQR GLSIARIEQL ENFRRAVLSL NRLLRHEIGT | |
| | KPEFGSSTCG ESLPDPCPDL TDKIVRLKEE RVNQTAHLII AQSLGVRLKG | |
| | HSLFTEEREK ADMHGEHEVI PGRSPVDFVV LEDLSRYTTD KSRSRSENSR | |
| | LMKWCHRKIN EKVKLLAEPF GIPVIEVFAS YSSKEDARTG APGFRAVEVT | |
| | SEDRPFWRKT IEKQSVAREV FDCLDNLVGK GLNGIHLVLP QNGGPLFIAA | |
| | VKEDQPLPAI RQADINAAVN IGLRAIAGPS CYHAHPKVRL IKGESGTDKG | |
| | KWLPRKGKEA NKRENAQFGN VDLDLEVKEN RLDIDSDVLK GDNTNLFHDP | |
| | LNIACYGFAT IQNLQHPFLA HASAVESRQK GAVARLQWEV CRAINSRRLE | |
| | AWQKKAEKAA VKR | |
| Phycisphaerae | MATKSYRARI LTDSRLAAAL DRTHVVFVES LKQMINTYLR MQNGKFGPDH | (SEQ |
| bacterium ST- | KKLAQIMLSR SNTFAHGVMD QITRDQPTST LDEEWTDLAR RIHKTTGPLF | ID |
| NAGAB-D1 | LQAERFATVK NRAIHTKSRG KVIPSPETLA VPAKFWHQVC DSASAYIRSN | NO: |
| (transposase) | RELMQQWRKD RAAWLKDKNE WQQKHPEFMQ FYNGPYQNFL KLCDDDRITS | 180) |
| AQT69685.1 | QLAAEQQPTA SKNNRPRKTG KRFARWHLWY KWLSENPEII EWRNKASASD | |
| | FKTVTDDVRK QIITKYPQQN KYITRLLDWL EDNNPELKTL ENLRRTYVKK | |
| | FDSFKRPPTL TLPSPYRHPY WFTMELDQFY KKADFENGTI QLLLIDEDDD | |
| | GNWFFNWMPA SLKPDPRLVP SWRAETFETE GREPPYLGGK IGKKLSRPAP | |
| | TDAERKAGIA GAKLMIKNNR SELLFTVFEQ DCPPRVKWAK TKNRKCPADN | |
| | AFSSDGKTRK PLRILSIDLG IRHIGAFALT QGTRNDSAWQ TESLKKGIIN | |
| | SPSIPPLRQV RRHDYDLKRK RRRHGKPVKG QRSNANLQAH RTNMAQDREK | |
| | KGASAIVSLA REHSADLILF ENLHSLKFSA FDERWMNRQL RDMNRRHIVE | |
| | LVSEQAPEFG ITVKDDINPW MTSRICSNCN LPGFRFSMKK KNPYREKLPR | |
| | EKCTDFGYPV WEPGGHLERC PHCDHRVNAD INAAANLANK FFGLGYWNNG | |
| | LKYDAETKTF TVHTDKKTPP LIFKPRPQFD LWADSVKTRK QLGPDPF | |
| Planctomycetes | MSVRSFQARV ECDKQTMEHL WRTHKVENER LPEIIKILFK MKRGECGQND | (SEQ |
| bacterium | KQKSLYKSIS QSILEANAQN ADYLLNSVSI KGWKPGTAKK YRNASFTWAD | ID |
| RBG_13_46_10 | DAAKLSSQGI HVYDKKQVLG DLPGMMSQMV CRQSVEAISG HIELTKKWEK | NO: |
| OHB62175.1 | EHNEWLKEKE KWESEDEHKK YLDLREKFEQ FEQSIGGKIT KRRGRWHLYL | 181) |
| | KWLSDNPDFA AWRGNKAVIN PLSEKAQIRI NKAKPNKKNS VERDEFFKAN | |
| | PEMKALDNLH GYYERNFVRR RKTKKNPDGF DHKPTFTLPH PTIHPRWFVE | |
| | NKPKTNPEGY RKLILPKKAG DLGSLEMRLL TGEKNKGNYP DDWISVKFKA | |
| | DPRLSLIRPV KGRRVVRKGK EQGQTKETDS YEFFDKHLKK WRPAKLSGVK | |
| | LIFPDKTPKA AYLYFTCDIP DEPLTETAKK IQWLETGDVT KKGKKRKKKV | |
| | LPHGLVSCAV DLSMRRGTTG FATLCRYENG KIHILRSRNL WVGYKEGKGC | |
| | HPYRWTEGPD LGHIAKHKRE IRILRSKRGK PVKGEESHID LQKHIDYMGE | |
| | DREKKAARTI VNFALNTENA ASKNGFYPRA DVLLLENLEG LIPDAEKERG | |
| | INRALAGWNR RHLVERVIEM AKDAGFKRRV FEIPPYGTSQ VCSKCGALGR | |
| | RYSIIRENNR REIRFGYVEK LFACPNCGYC ANADHNASVN LNRRELIEDS | |
| | FKSYYDWKRL SEKKQKEEIE TIESKLMDKL CAMHKISRGS ISK | |
| Spirochaetes | MSFTISYPFK LIIKNKDEAK ALLDTHQYMN EGVKYYLEKL LMFRQEKIFI | (SEQ |
| bacterium | GEDETGKRIY IEETEYKKQI EEFYLIKKTE LGRNLTLTLD EFKTLMRELY | ID |
| GWB1_27_13 | ICLVSSSMEN KKGFPNAQQA SLNIFSPLED AESKGYILKE ENNNISLIHK | NO: |
| OHD16008.1 | DYGKILLKRL RDNNLIPIFT KFTDIKKITA KLSPTALDRM IFAQAIEKLL | 182) |
| | SYESWCKLMI KERFDKEVKI KELENKCENK QERDKIFEIL EKYEEERQKT | |
| | FEQDSGFAKK GKFYITGRML KGFDEIKEKW LKEKDRSEQN LINILNKYQT | |
| | DNSKLVGDRN LFEFIIKLEN QCLWNGDIDY LKIKRDINKN QIWLDRPEMP | |
| | RFTMPDFKKH PLWYRYEDPS NSNFRNYKIE VVKDENYITI PLITERNNEY | |
| | FEENYTENLA KLKKLSENIT FIPKSKNKEF EFIDSNDEEE DKKDQKKSKQ | |
| | YIKYCDTAKN TSYGKSGGIR LYENRNELEN YKDGKKMDSY TVFTLSIRDY | |
| | KSLFAKEKLQ PQIFNTVDNK ITSLKIQKKF GNEEQTNELS YFTQNQITKK | |
| | DWMDEKTFQN VKELNEGIRV LSVDLGQRFF AAVSCFEIMS EIDNNKLFEN | |
| | LNDQNHKIIR INDKNYYAKH IYSKTIKLSG EDDDLYKERK INKNYKLSYQ | |
| | ERKNKIGIFT RQINKLNQLL KIIRNDEIDK EKFKELIETT KRYVKNTYND | |
| | GIIDWNNVDN KILSYENKED VINLHKELDK KLEIDFKEFI RECRKPIFRS | |
| | GGLSMQRIDE LEKLNKLKRK WVARTQKSAE SIVLTPKEGY KLKEHINELK | |
| | DNRVKQGVNY ILMTALGYIK DNEIKNDSKK KQKEDWVKKN RACQIILMEK | |
| | LTEYTFAEDR PREENSKLRM WSHRQIFNFL QQKASLWGIL VGDVFAPYTS | |
| | KCLSDNNAPG IRCHQVTKKD LIDNSWFLKI VVKDDAFCDL IEINKENVKN | |
| | KSIKINDILP LRGGELFASI KDGKLHIVQA DINASRNIAK RFLSQINPER | |
| | VVLKKDKDET FHLKNEPNYL KNYYSILNFV PTNEELTFFK VEENKDIKPT | |
| | KRIKMDKHEK ESTDEGDDYS KNQIALFRDD SGIFFDKSLW VDGKIFWSVV | |
| | KNKMTKLLRE RNNKKNGSK | |
| Verrucomicrobiaceae | MPLSRIYQGR TNSLIILTPT PQEPWDHKAL AREDSPLWRH HALFQDAVNY | (SEQ |
| bacterium | YQLCLVALAS SDGTRPLSKL HEQMKASWDE AKTDTEDSWR VRLARRLGIP | ID |
| UBA2429 | AASLFEAALA KVLEGNEAPE RARELAGELL LDKIEGDIQQ AGRGYWPRFC | NO: |
| GCA_002343505.1 | DPKANPTYDY SATARASASG LTKLAAVIHA ENVTEEALKQ VAAEMDLSWT | 183) |
| | VKLQPDKNFV GAEARARLLE AAHHFIKVAE SPPTKLAEVL ARFPDGLALW | |
| | QALPEKIAAL PEETQVPRNR NAVTQAVVIE EPEIDFAELG DDPIKLARGE | |
| | KPKSVKAPKV VEKVSARRKA KASPDLTFAT LLFQHFPSLF TAAVLGLSVG | |
| | RGFVFPAFTS LSFWAVPGPH VPVWKEFDIA AFKEALKTVN QFKLKTSERN | |
| | ALLAEAQRRL DYMDEKTHDW KTGDSDEPGH IPPRLKSDPN FTLIQALTQD | |
| | EGVSNKATGD QHIPKGVYTG GLRGFYAIKK DWCELWERKA DKSQGTPTEE | |
| | ELISIVTDYQ RDHVYDVGDV GLFRALCEPR FWPLWQPLTD EQEAERIKAG | |
| | RAKDMISAYR VWLELQEDVV RLAQPIRFTP AHAENSRRLF MESDISGSHG | |
| | AEFGSDGKSL EVSIAYDVDG KLQPVRAKLE FSAPRAARDE LEGLSGGSES | |
| | MRWFQPMMKA LDCPEVEMPA LEKCAVSLMP DVVKKGGGKW VRLLLNFPAT | |
| | LEPEGLIRHI GKQAMWYKQF NGTYKPRTQQ LDTGLHLYWP GLEKAPEAED | |
| | AAAWWNREEI RAKGFSVLSV DLGQRDAGAW ALLESRSDKA FSRNRQPFIE | |
| | LGEAGGKLWS TALLGLGMLR LPGEDARTGA LDDQGKRAVE FHGKAGRNAL | |
| | EAEWQEAREM ALLFGGEEAK SRLGPGEDHL SHSKQNEELL RILSRAQSRL | |
| | ARFHRWSCRI HEKPEATGDD VIDYGQVDEL LTKTAEAMLE NLKALYTNAG | |
| | GILDSKSKQP LTLVGLRKKL EAQKVEPEKI AAVLKPHAEI IFQRLGTLIP | |
| | ELKQHLRVSL ERLANRELPL RHREWVWNEA FEKLEQGNEK KEENPKWIRG | |
| | QRGLSMARIE QIENLRKREM SLRRQMSLIP GEQVKQGVED KGQRQPEPCE | |
| | DILNKLDRMK QQRVNQTAHL ILAQALGIRL RPHLANDAER EEKDIHGEYE | |
| | LIPGRKPVDF IVMEDLSRYL SSQGRAPSEN GRLMKWCHRA VLAKLKQMCE | |
| | PFGIPVLEVP AAYSSRFCAL TGVPGFRAVE VHDGNAEDER WKRLIKKAEK | |
| | DKSSKDAEAA AMLFDQLHDL NIEAREARKQ DKKLPLRTLF APVAGGPLFI | |
| | PMVGGGPRQA DMNAAINLGL RAIASPTCLR ARPKIRAELK DGKHQAMLGN | |
| | KLEKAAALTL EPPKEPTKEL AAQKRTNEFL DEKFVGKEDT AHVTTSGKKL | |
| | RLSGGMSLWK AIKDGAWQRV KKINDARIAK WKNNPPPEPD PDDEIQF | |
| Alicyclobacillus | MAVKSIKVKL RLSECPDILA GMWQLHRATN AGVRYYTEWV SLMRQEILYS | (SEQ |
| kakegawensis | RGPDGGQQCY MTAEDCQREL LRRLRNRQLH NGRQDQPGTD ADLLAISRRL | ID |
| WP_067936067.1 | YEILVLQSIG KRGDAQQIAS SFLSPLVDPN SKGGRGEAKS GRKPAWQKMR | NO: |
| | DQGDPRWVAA REKYEQRKAV DPSKEILNSL DALGLRPLFA VFTETYRSGV | 184) |
| | DWKPLGKSQG VRTWDRDMFQ QALERLMSWE SWNRRVGEEY ARLFQQKMKE | |
| | EQEHFAEQSH LVKLARALEA DMRAASQGFE AKRGTAHQIT RRALRGADRV | |
| | FEIWKSIPEE ALFSQYDEVI RQVQAEKRRD FGSHDLFAKL AEPKYQPLWR | |
| | ADETFLTRYA LYNGVLRDLE KARQFATFTL PDACVNPIWT RFESSQGSNL | |
| | HKYEFLEDHL GPGRHAVRFQ RLLVVESEGA KERDSVVVPV APSGQLDKLV | |
| | LREEEKSSVA LHLHDTARPD GFMAEWAGAK LQYERSTLAR KARRDKQGMR | |
| | SWRRQPSMLM SAAQMLEDAK QAGDVYLNIS VRVKSPSEVR GQRRPPYAAL | |
| | FRIDDKQRRV TVNYNKLSAY LEEHPDKQIP GAPGLLSGLR VMSVDLGLRT | |
| | SASISVERVA KKEEVEALGD GRPPHYYPIH GTDDLVAVHE RSHLIQMPGE | |
| | TETKQLRKLR EERQAVLRPL FAQLALLRLL VRCGAADERI RTRSWQRLTK | |
| | QGREFTKRLT PSWREALELE LTRLEAYCGR VPDDEWSRIV DRTVIALWRR | |
| | MGKQVRDWRK QVKSGAKVKV KGYQLDVVGG NSLAQIDYLE QQYKFLRRWS | |
| | FFARASGLVV RADRESHFAV ALRQHIENAK RDRLKKLADR ILMEALGYVY | |
| | EASGPREGQW TAQHPPCQLI ILEELSAYRE SDDRPPSENS KLMAWGHRGI | |
| | LEELVNQAQV HDVLVGTVYA AFSSRFDART GAPGVRCRRV PARFVGATVD | |
| | DSLPLWLTEF LDKHRLDKNL LRPDDVIPTG EGEFLVSPCG EEAARVRQVH | |
| | ADINAAQNLQ RRLWQNEDIT ELRLRCDVKM GGEGTVLVPR VNNARAKQLF | |
| | GKKVLVSQDG VTFFERSQTG GKPHSEKQTD LTDKELELIA EADEARAKSV | |
| | VLFRDPSGHI GKGHWIRQRE FWSLVKQRIE SHTAERIRVR GVGSSLD | |
| Bacillus sp._ | MAIRSIKLKM KTNSGTDSIY LRKALWRTHQ LINEGIAYYM NLLTLYRQEA | (SEQ |
| V3-13 | IGDKTKEAYQ AELINIIRNQ QRNNGSSEEH GSDQEILALL RQLYELIIPS | ID |
| WP_101661451.1 | SIGESGDANQ LGNKFLYPLV DPNSQSGKGT SNAGRKPRWK RLKEEGNPDW | NO: |
| | ELEKKKDEER KAKDPTVKIF DNLNKYGLLP LFPLETNIQK DIEWLPLGKR | 185) |
| | QSVRKWDKDM FIQAIERLLS WESWNRRVAD EYKQLKEKTE SYYKEHLTGG | |
| | EEWIEKIRKF EKERNMELEK NAFAPNDGYF ITSRQIRGWD RVYEKWSKLP | |
| | ESASPEELWK VVAEQQNKMS EGFGDPKVES FLANRENRDI WRGHSERIYH | |
| | IAAYNGLQKK LSRTKEQATF TLPDAIEHPL WIRYESPGGT NLNLFKLEEK | |
| | QKKNYYVTLS KIIWPSEEKW IEKENIEIPL APSIQFNRQI KLKQHVKGKQ | |
| | EISFSDYSSR ISLDGVLGGS RIQFNRKYIK NHKELLGEGD IGPVFFNLVV | |
| | DVAPLQETRN GRLQSPIGKA LKVISSDESK VIDYKPKELM DWMNTGSASN | |
| | SFGVASLLEG MRVMSIDMGQ RTSASVSIFE VVKELPKDQE QKLFYSINDT | |
| | ELFAIHKRSF LLNLPGEVVT KNNKQQRQER RKKRQFVRSQ IRMLANVLRL | |
| | ETKKTPDERK KAIHKLMEIV QSYDSWTASQ KEVWEKELNL LTNMAAFNDE | |
| | IWKESLVELH HRIEPYVGQI VSKWRKGLSE GRKNLAGISM WNIDELEDTR | |
| | RLLISWSKRS RTPGEANRIE TDEPFGSSLL QHIQNVKDDR LKQMANLIIM | |
| | TALGFKYDKE EKDRYKRWKE TYPACQIILF ENLNRYLENL DRSRRENSRL | |
| | MKWAHRSIPR TVSMQGEMFG LQVGDVRSEY SSRFHAKTGA PGIRCHALTE | |
| | EDLKAGSNTL KRLIEDGFIN ESELAYLKKG DIIPSQGGEL FVTLSKRYKK | |
| | DSDNNELTVI HADINAAQNL QKREWQQNSE VYRVPCQLAR MGEDKLYIPK | |
| | SQTETIKKYF GKGSFVKNNT EQEVYKWEKS EKMKIKTDTT FDLQDLDGFE | |
| | DISKTIELAQ EQQKKYLTMF RDPSGYFENN ETWRPQKEYW SIVNNIIKSC | |
| | LKKKILSNKV EL | |
| Desulfatirhabdium | MPLSNNPPVT QRAYTLRLRG ADPSDLSWRE ALWHTHEAVN KGAKVFGDWL | (SEQ |
| butyrativorans | LTLRGGLDHT LADTKVKGGK GKPDRDPTPE ERKARRILLA LSWLSVESKL | ID |
| WP_028326052.1 | GAPSSYIVAS GDEPAKDRND NVVSALEEIL QSRKVAKSEI DDWKRDCSAS | NO: |
| | LSAAIRDDAV WVNRSKVEDE AVKSVGSSLT REEAWDMLER FFGSRDAYLT | 186) |
| | PMKDPEDKSS ETEQEDKAKD LVQKAGQWLS SRYGTSEGAD FCRMSDIYGK | |
| | IAAWADNASQ GGSSTVDDLV SELRQHEDTK ESKATNGLDW IIGLSSYTGH | |
| | TPNPVHELLR QNTSLNKSHL DDLKKKANTR AESCKSKIGS KGQRPYSDAI | |
| | LNDVESVCGF TYRVDKDGQP VSVADYSKYD VDYKWGTARH YIFAVMLDHA | |
| | ARRISLAHKW IKRAEAERHK FEEDAKRIAN VPARAREWLD SFCKERSVTS | |
| | GAVEPYRIRR RAVDGWKEVV AAWSKSDCKS TEDRIAAARA LQDDSEIDKE | |
| | GDIQLFEALA EDDALCVWHK DGEATNEPDF QPLIDYSLAI EAEFKKRQFK | |
| | VPAYRHPDEL LHPVFCDFGK SRWKINYDVH KNVQAPFYRG LCLTLWTGSE | |
| | IKPVPLCWQS KRLTRDLALG NNHRNDAASA VTRADRLGRA ASNVTKSDMV | |
| | NITGLFEQAD WNGRLQAPRQ QLEAIAVVRD NPRLSEQERN LRMCGMIEHI | |
| | RWLVTFSVKL QPQGPWCAYA EQHGLNTNPQ YWPHADTNRD RKVHARLILP | |
| | RLPGLRVLSV DLGHRYAAAC AVWEAVNTET VKEACQNVGR DMPKEHDLYL | |
| | HIKVKKQGIG KQTEVDKTTI YRRIGADTLP LIDRLIASGW GLLKRQMARL | |
| | QGEEKDAREA SNEEIWALHQ MECKLDRTKP DGRPHPAPWA RLDRQFLIKL | |
| | DALKELGWIP APDSSENLSR EDGEAKDYRE SLAVDDLMES AVRTLRLALQ | |
| | RHGNRARIAY YLISEVKIRP GGIQEKLDEN GRIDLLQDAL ALWHELESSP | |
| | GWRDEAAKQL WDSRIATLAG YKAPEENGDN VSDVAYRKKQ QVYREQLRNV | |
| | AKTLSGDVIT CKELSDAWKE RWEDEDQRWK KLLRWFKDWV LPSGTQANNA | |
| | TIRNVGGLSL SRLATITEFR RKVQVGFFTR LRPDGTRHEI GEQFGQKTLD | |
| | ALELLREQRV KQLASRIAEA ALGIGSEGGK GWDGGKRPRQ RINDSRFAPC | |
| | HAVVIENLAN YRPDETRTRL ENRRLMTWSA SKVHKYLSEA CQLNGLYLCT | |
| | VSAWYTSRQD SRTGAPGIRC QDVSVREFMQ SPFWRKQVKQ AEAKHDENKG | |
| | DARERELCEL NKTWKAKTPA EWKKAGFVRI PLRGGEIFVS ADSKSPSAKG | |
| | IHADLNAAAN IGLRALTDPD WPGKWWYVPC DPVSFESKMD YVKGCAAVKV | |
| | GQPLRQPAQT NADGAASKIR KGKKNRTAGT SKEKVYLWRD ISAFPLESNE | |
| | IGEWKETSAY QNDVQYRVIR MLKEHIKSLD NRTGDNVEG | |
| Desulfonatronum | MVLGRKDDTA ELRRALWITH EHVNLAVAEV ERVLLRCRGR SYWTLDRRGD | (SEQ |
| thiodismutans | PVHVPESQVA EDALAMAREA QRRNGWPVVG EDEEILLALR YLYEQIVPSC | ID |
| WP_031386437.1 | LLDDLGKPLK GDAQKIGTNY AGPLFDSDTC RRDEGKDVAC CGPFHEVAGK | NO: |
| | YLGALPEWAT PISKQEFDGK DASHLRFKAT GGDDAFFRVS IEKANAWYED | 187) |
| | PANQDALKNK AYNKDDWKKE KDKGISSWAV KYIQKQLQLG QDPRTEVRRK | |
| | LWLELGLLPL FIPVEDKTMV GNLWNRLAVR LALAHLLSWE SWNHRAVQDQ | |
| | ALARAKRDEL AALFLGMEDG FAGLREYELR RNESIKQHAF EPVDRPYVVS | |
| | GRALRSWTRV REEWLRHGDT QESRKNICNR LQDRLRGKFG DPDVFHWLAE | |
| | DGQEALWKER DCVTSFSLLN DADGLLEKRK GYALMTFADA RLHPRWAMYE | |
| | APGGSNLRTY QIRKTENGLW ADVVLLSPRN ESAAVEEKTE NVRLAPSGQL | |
| | SNVSFDQIQK GSKMVGRCRY QSANQQFEGL LGGAEILEDR KRIANEQHGA | |
| | TDLASKPGHV WFKLTLDVRP QAPQGWLDGK GRPALPPEAK HEKTALSNKS | |
| | KFADQVRPGL RVLSVDLGVR SFAACSVFEL VRGGPDQGTY FPAADGRTVD | |
| | DPEKLWAKHE RSFKITLPGE NPSRKEEIAR RAAMEELRSL NGDIRRLKAI | |
| | LRLSVLQEDD PRTEHLRLFM EAIVDDPAKS ALNAELFKGF GDDRERSTPD | |
| | LWKQHCHFFH DKAEKVVAER FSRWRTETRP KSSSWQDWRE RRGYAGGKSY | |
| | WAVTYLEAVR GLILRWNMRG RTYGEVNRQD KKQFGTVASA LLHHINQLKE | |
| | DRIKTGADMI IQAARGFVPR KNGAGWVQVH EPCRLILFED LARYRERTDR | |
| | SRRENSRLMR WSHREIVNEV GMQGELYGLH VDTTEAGESS RYLASSGAPG | |
| | VRCRHLVEED FHDGLPGMHL VGELDWLLPK DKDRTANEAR RLLGGMVRPG | |
| | MLVPWDGGEL FATLNAASQL HVIHADINAA QNLQRREWGR CGEAIRIVCN | |
| | MTPTNAGKKY EMAKAPKARL LGALQQLKNG DAPFHLTSIP NSQKPENSYV | |
| | QLSVDGSTRY RAGPGEKSSG EEDELALDIV EQAEELAQGR KTFFRDPSGV | |
| | FFAPDRWLPS EIYWSRIRRR IWQVTLERNS SGRQERAEMD EMPY | |
| Lentisphaeria | MAVELNRIYQ GRVNHVYIFD ENQNQVSVDN GDDLLFVHHE LYQDAINYYL | (SEQ |
| bacterium | VALAAMALDS KDSLFGKEKM QIRAVWNDFY RNGQLRPGLK HSLIRSLGHA | ID |
| DCFZ01000012.1 | AELNTSNGAD IAMNLILEDG GIPSEILNAA LEHLAEKCTG DVSQLGKTFF | NO: |
| | PRFCDTAYHG NWDVDAKSES EKKGRQRLVD ALYSLHPVQA VQELAPEIEI | 188) |
| | GWGGVKTQTG KFFTGDEAKA SLKKAISYFL QDTGKNSPEL QEYFSVAGKQ | |
| | PLEQYLGKID TFPEISFGRI SSHQNINISN AMWILKFFPD QYSVDLIKNL | |
| | IPNKKYEIGI APQWGDDPVK LSRGKRGYTF RAFTDLAMWE KNWKVEDRAA | |
| | FSDALKTINQ FRNKTQERND QLKRYCAALN WMDGESSDKK PPVEPADADA | |
| | VDEAATSVLP ILAGDKRWNA LLQLQKELGI CNDFTENELM DYGLSLRTIR | |
| | GYQKLRSMML EKEEKMRAKT ADDEEISQAL QEIIIKFQSS HRDTIGSVSL | |
| | FLKLAEPKYF CVWHDADKNQ NFASVDMVAD AVRYYSYQEE KARLEEPIQI | |
| | TPADARYSRR VSDLYALVYK NAKECKTGYG LRPDGNFVFE IAQKNAKGYA | |
| | PAKVVLAFSA PRLKRDGLID KEFSAYYPPV LQAFLREEEA PKQSFKTTAV | |
| | ILMPDWDKNG KRRILLNFPI KLDVSAIHQK TDHRFENQFY FANNTNTCLL | |
| | WPSYQYKKPV TWYQGKKPFD VVAVDLGQRS AGAVSRITVS TEKREHSVAI | |
| | GEAGGTQWYA YRKESGLLRL PGEDATVIRD GQRTEELSGN AGRLSTEEET | |
| | VQACVLCKML IGDATLLGGS DEKTIRSFPK QNDKLLIAFR RATGRMKQLQ | |
| | RWLWMLNENG LCDKAKTEIS NSDWLVNKNI DNVLKEEKQH REMLPAILLQ | |
| | IADRVLPLRG RKWDWVLNPQ SNSFVLQQTA HGSGDPHKKI CGQRGLSFAR | |
| | IEQLESLRMR CQALNRILMR KTGEKPATLA EMRNNPIPDC CPDILMRLDA | |
| | MKEQRINQTA NLILAQALGL RHCLHSESAT KRKENGMHGE YEKIPGVEPA | |
| | AFVVLEDLSR YRESQDRSSY ENSRLMKWSH RKILEKLALL CEVENVPILQ | |
| | VGAAYSSKES ANAIPGFRAE ECSIDQLSFY PWRELKDSRE KALVEQIRKI | |
| | GHRLLTFDAK ATIIMPRNGG PVFIPFVPSD SKDTLIQADI NASENIGLRG | |
| | VADATNLLCN NRVSCDRKKD CWQVKRSSNF SKMVYPEKLS LSFDPIKKQE | |
| | GAGGNFFVLG CSERILTGTS EKSPVFTSSE MAKKYPNLME GSALWRNEIL | |
| | KLERCCKINQ SRLDKFIAKK EVQNEL | |
| Laceyella | MSIRSFKLKI KTKSGVNAEE LRRGLWRTHQ LINDGIAYYM NWLVLLRQED | (SEQ |
| sediminis | LFIRNEETNE IEKRSKEEIQ GELLERVHKQ QQRNQWSGEV DDQTLLQTLR | ID |
| WP_106341859.1 | HLYEEIVPSV IGKSGNASLK ARFFLGPLVD PNNKTTKDVS KSGPTPKWKK | NO: |
| | MKDAGDPNWV QEYEKYMAER QTLVRLEEMG LIPLFPMYTD EVGDIHWLPQ | 189) |
| | ASGYTRTWDR DMFQQAIERL LSWESWNRRV RERRAQFEKK THDFASRESE | |
| | SDVQWMNKLR EYEAQQEKSL EENAFAPNEP YALTKKALRG WERVYHSWMR | |
| | LDSAASEEAY WQEVATCQTA MRGEFGDPAI YQFLAQKENH DIWRGYPERV | |
| | IDFAELNHLQ RELRRAKEDA TFTLPDSVDH PLWVRYEAPG GTNIHGYDLV | |
| | QDTKRNLTLI LDKFILPDEN GSWHEVKKVP FSLAKSKQFH RQVWLQEEQK | |
| | QKKREVVFYD YSTNLPHLGT LAGAKLQWDR NELNKRTQQQ IEETGEIGKV | |
| | FFNISVDVRP AVEVKNGRLQ NGLGKALTVL THPDGTKIVT GWKAEQLEKW | |
| | VGESGRVSSL GLDSLSEGLR VMSIDLGQRT SATVSVFEIT KEAPDNPYKF | |
| | FYQLEGTELF AVHQRSFLLA LPGENPPQKI KQMREIRWKE RNRIKQQVDQ | |
| | LSAILRLHKK VNEDERIQAI DKLLQKVASW QLNEEIATAW NQALSQLYSK | |
| | AKENDLQWNQ AIKNAHHQLE PVVGKQISLW RKDLSTGRQG IAGLSLWSIE | |
| | ELEATKKLLT RWSKRSREPG VVKRIERFET FAKQIQHHIN QVKENRLKQL | |
| | ANLIVMTALG YKYDQEQKKW IEVYPACQVV LFENLRSYRE SYERSRRENK | |
| | KLMEWSHRSI PKLVQMQGEL FGLQVADVYA AYSSRYHGRT GAPGIRCHAL | |
| | TEADLRNETN IIHELIEAGF IKEEHRPYLQ QGDLVPWSGG ELFATLQKPY | |
| | DNPRILTLHA DINAAQNIQK RFWHPSMWER VNCESVMEGE IVTYVPKNKT | |
| | VHKKQGKTFR FVKVEGSDVY EWAKWSKNRN KNTFSSITER KPPSSMILER | |
| | DPSGTFFKEQ EWVEQKTFWG KVQSMIQAYM KKTIVQRMEE | |
| Methylobacterium | MYEAIVLADD ANAQLANAFL GPLTDPNSAG FLEAFNKVDR PAPSWLDQVP | (SEQ |
| nodulans | ASDPIDPAVL AEANAWLDTD AGRAWLVDTG APPRWRSLAA KQDPIWPREF | ID |
| (long form) | ARKLGELRKE AASGTSAIIK ALKRDFGVLP LFQPSLAPRI LGSRSSLTPW | NO: |
| | DRLAFRLAVG HLLSWESWCT RARDEHTARV QRLEQFSSAH LKGDLATKVS | 190) |
| | TLREYERARK EQIAQLGLPM GERDFLITVR MTRGWDDLRE KWRRSGDKGQ | |
| | EALHAIIATE QTRKRGREGD PDLERWLARP ENHHVWADGH ADAVGVLARV | |
| | NAMERLVERS RDTALMTLPD PVAHPRSAQW EAEGGSNLRN YQLEAVGGEL | |
| | QITLPLLKAA DDGRCIDTPL SFSLAPSDQL QGVVLTKQDK QQKITYCTNM | |
| | NEVFEAKLGS ADLLLNWDHL RGRIRDRVDA GDIGSAFLKL ALDVAHVLPD | |
| | GVDDQLARAA FHFQSAKGAK SKHADSVQAG LRVLSIDLGV RSFATCSVFE | |
| | LKDTAPTTGV AFPLAEFRLW AVHERSFTLE LPGENVGAAG QQWRAQADAE | |
| | LRQLRGGLNR HRQLLRAATV QKGERDAYLT DLREAWSAKE LWPFEASLLS | |
| | ELERCSTVAD PLWQDTCKRA ARLYRTEFGA VVSEWRSRTR SREDRKYAGK | |
| | SMWSVQHLTD VRRFLQSWSL AGRASGDIRR LDRERGGVFA KDLLDHIDAL | |
| | KDDRLKTGAD LIVQAARGFQ RNEFGYWVQK HAPCHVILFE DLSRYRMRTD | |
| | RPRRENSQLM QWAHRGVPDM VGMQGEIYGI QDRRDPDSAR KHARQPLAAF | |
| | CLDTPAAFSS RYHASTMTPG IRCHPLRKRE FEDQGFLELL KRENEGLDLN | |
| | GYKPGDLVPL PGGEVFVCLN ANGLSRIHAD INAAQNLQRR FWTQHGDAFR | |
| | LPCGKSAVQG QIRWAPLSMG KRQAGALGGF GYLEPTGHDS GSCQWRKTTE | |
| | AEWRRLSGAQ KDRDEAAAAE DEELQGLEEE LLERSGERVV FFRDPSGVVL | |
| | PTDLWFPSAA FWSIVRAKTV GRLRSHLDAQ AEASYAVAAG L | |
| Opitutaceae | MSLNRIYQGR VAAVETGTAL AKGNVEWMPA AGGDEVLWQH HELFQAAINY | (SEQ |
| bacterium | YLVALLALAD KNNPVLGPLI SQMDNPQSPY HVWGSFRRQG RQRTGLSQAV | ID |
| WP_009513281.1 | APYITPGNNA PTLDEVERSI LAGNPTDRAT LDAALMQLLK ACDGAGAIQQ | NO: |
| | EGRSYWPKFC DPDSTANFAG DPAMLRREQH RLLLPQVLHD PAITHDSPAL | 191) |
| | GSFDTYSIAT PDTRTPQLTG PKARARLEQA ITLWRVRLPE SAADEDRLAS | |
| | SLKKIPDDDS RLNLQGYVGS SAKGEVQARL FALLLFRHLE RSSFTLGLLR | |
| | SATPPPKNAE TPPPAGVPLP AASAADPVRI ARGKRSFVER AFTSLPCWHG | |
| | GDNIHPTWKS FDIAAFKYAL TVINQIEEKT KERQKECAEL ETDEDYMHGR | |
| | LAKIPVKYTT GEAEPPPILA NDLRIPLLRE LLQNIKVDTA LTDGEAVSYG | |
| | LQRRTIRGFR ELRRIWRGHA PAGTVESSEL KEKLAGELRQ FQTDNSTTIG | |
| | SVQLENELIQ NPKYWPIWQA PDVETARQWA DAGFADDPLA ALVQEAELQE | |
| | DIDALKAPVK LTPADPEYSR RQYDENAVSK FGAGSRSANR HEPGQTERGH | |
| | NTFTTEIAAR NAADGNRWRA THVRIHYSAP RLLRDGLRRP DTDGNEALEA | |
| | VPWLQPMMEA LAPLPTLPQD LTGMPVELMP DVTLSGERRI LLNLPVTLEP | |
| | AALVEQLGNA GRWQNQFFGS REDPFALRWP ADGAVKTAKG KTHIPWHQDR | |
| | DHFTVLGVDL GTRDAGALAL LNVTAQKPAK PVHRIIGEAD GRTWYASLAD | |
| | ARMIRLPGED ARLFVRGKLV QEPYGERGRN ASLLEWEDAR NIILRLGQNP | |
| | DELLGADPRR HSYPEINDKL LVALRRAQAR LARLQNRSWR LRDLAESDKA | |
| | LDEIHAERAG EKPSPLPPLA RDDAIKSTDE ALLSQRDIIR RSFVQIANLI | |
| | LPLRGRRWEW RPHVEVPDCH ILAQSDPGTD DTKRIVAGQR GISHERIEQI | |
| | EELRRRCQSL NRALRHKPGE RPVLGRPAKG EEIADPCPAL LEKINRLRDQ | |
| | RVDQTAHAIL AAALGVRLRA PSKDRAERRH RDIHGEYERF RAPADFVVIE | |
| | NLSRYLSSQD RARSENTRLM QWCHRQIVQK LRQLCETYGI PVLAVPAAYS | |
| | SRESSRDGSA GFRAVHLTPD HRHRMPWSRI LARLKAHEED GKRLEKTVLD | |
| | EARAVRGLED RLDRENAGHV PGKPWRTLLA PLPGGPVFVP LGDATPMQAD | |
| | LNAAINIALR GIAAPDRHDI HHRLRAENKK RILSLRLGTQ REKARWPGGA | |
| | PAVTLSTPNN GASPEDSDAL PERVSNLFVD IAGVANFERV TIEGVSQKFA | |
| | TGRGLWASVK QRAWNRVARL NETVTDNNRN EEEDDIPM | |
| Thermomonas | MSEKTTQRAY TLRLNRASGE CAVCQNNSCD CWHDALWATH KAVNRGAKAF | (SEQ |
| hydrothermalis | GDWLLTLRGG LCHTLVEMEV PAKGNNPPQR PTDQERRDRR VLLALSWLSV | ID |
| WP_072754838.1 | EDEHGAPKEF IVATGRDSAD DRAKKVEEKL REILEKRDFQ EHEIDAWLQD | NO: |
| | CGPSLKAHIR EDAVWVNRRA LEDAAVERIK TLTWEEAWDF LEPFFGTQYF | 192) |
| | AGIGDGKDKD DAEGPARQGE KAKDLVQKAG QWLSARFGIG TGADEMSMAE | |
| | AYEKIAKWAS QAQNGDNGKA TIEKLACALR PSEPPTLDTV LKCISGPGHK | |
| | SATREYLKTL DKKSTVTQED LNQLRKLADE DARNCRKKVG KKGKKPWADE | |
| | VLKDVENSCE LTYLQDNSPA RHREFSVMLD HAARRVSMAH SWIKKAEQRR | |
| | RQFESDAQKL KNLQERAPSA VEWLDRFCES RSMTTGANTG SGYRIRKRAI | |
| | EGWSYVVQAW AEASCDTEDK RIAAARKVQA DPEIEKFGDI QLFEALAADE | |
| | AICVWRDQEG TQNPSILIDY VTGKTAEHNQ KRFKVPAYRH PDELRHPVEC | |
| | DFGNSRWSIQ FAIHKEIRDR DKGAKQDTRQ LQNRHGLKMR LWNGRSMTDV | |
| | NLHWSSKRLT ADLALDQNPN PNPTEVTRAD RLGRAASSAF DHVKIKNVEN | |
| | EKEWNGRLQA PRAELDRIAK LEEQGKTEQA EKLRKRLRWY VSFSPCLSPS | |
| | GPFIVYAGQH NIQPKRSGQY APHAQANKGR ARLAQLILSR LPDLRILSVD | |
| | LGHRFAAACA VWETLSSDAF RREIQGLNVL AGGSGEGDLF LHVEMTGDDG | |
| | KRRTVVYRRI GPDQLLDNTP HPAPWARLDR QFLIKLQGED EGVREASNEE | |
| | LWTVHKLEVE VGRTVPLIDR MVRSGFGKTE KQKERLKKLR ELGWISAMPN | |
| | EPSAETDEKE GEIRSISRSV DELMSSALGT LRLALKRHGN RARIAFAMTA | |
| | DYKPMPGGQK YYFHEAKEAS KNDDETKRRD NQIEFLQDAL SLWHDLESSP | |
| | DWEDNEAKKL WQNHIATLPN YQTPEEISAE LKRVERNKKR KENRDKLRTA | |
| | AKALAENDQL RQHLHDTWKE RWESDDQQWK ERLRSLKDWI FPRGKAEDNP | |
| | SIRHVGGLSI TRINTISGLY QILKAFKMRP EPDDLRKNIP QKGDDELENE | |
| | NRRLLEARDR LREQRVKQLA SRIIEAALGV GRIKIPKNGK LPKRPRTTVD | |
| | TPCHAVVIES LKTYRPDDLR TRRENRQLMQ WSSAKVRKYL KEGCELYGLH | |
| | FLEVPANYTS RQCSRTGLPG IRCDDVPTGD FLKAPWWRRA INTAREKNGG | |
| | DAKDRFLVDL YDHLNNLQSK GEALPATVRV PRQGGNLFIA GAQLDDTNKE | |
| | RRAIQADLNA AANIGLRALL DPDWRGRWWY VPCKDGTSEP ALDRIEGSTA | |
| | FNDVRSLPTG DNSSRRAPRE IENLWRDPSG DSLESGTWSP TRAYWDTVQS | |
| | RVIELLRRHA GLPTS | |
| Methylobacterium | MYEAIVLADD ANAQLANAFL GPLTDPNSAG FLEAFNKVDR PAPSWLDQVP | (SEQ |
| nodulans | ASDPIDPAVL AEANAWLDTD AGRAWLVDTG APPRWRSLAA KQDPIWPREF | ID |
| WP_043747912.1 | ARKLGELRKE AASGTSAIIK ALKRDFGVLP LFQPSLAPRI LGSRSSLTPW | NO: |
| | DRLAFRLAVG HLLSWESWCT RARDEHTARV QRLEQFSSAH LKGDLATKVS | 193) |
| | TLREYERARK EQIAQLGLPM GERDFLITVR MTRGWDDLRE KWRRSGDKGQ | |
| | EALHAIIATE QTRKRGREGD PDLFRWLARP ENHHVWADGH ADAVGVLARV | |
| | NAMERLVERS RDTALMTLPD PVAHPRSAQW EAEGGSNLRN YQLEAVGGEL | |
| | QITLPLLKAA DDGRCIDTPL | |
| Chloracidobacterium | MPQQAKPPVT QRAYTLRLRG ADSNDPSWRD ALWQTHEAVN RGAQAFGDWL | (SEQ |
| thermophilum | LTLRGGLDHT LADTPVKGGK GKPDPDPTDE ERKARRILLA LSWLSVESKL | ID |
| WP_058868187.1 | GAPAGLIIAF GTEAAEERNR KVVAALEEIL KSRGVDQNEI NAWKKDCSAS | NO: |
| | LSAAIRDDAV WVNRSKAFDE AVESIGSSGS SGSSLTREEP WDMLERFFGS | 194) |
| | RDAYLAPAKG SEDESSEAKQ EDQAKDLVQK AGQWLSSRFG TGKGADERRM | |
| | ATVYEAIAKW DGKASLEMAG DKAIADLATA LSEFNPASND LQGVLGLISG | |
| | PGYKSATRNF LNQLAAQTTV TQQDFVSLKD KANNDAQECK QNTGSKGQRP | |
| | YSNSILEKVE SVCGFTYLQD GGPARHSEFA VILDHAARRV SLAHTWIKLA | |
| | EAERRKFEED AKKIDQVPEA AKDWLDRFCL ERSGVSGALE PYRIRRRAVD | |
| | GWKEVVAEWS KSDCKTVEDR IAAARALQDD PEIDKFGDIQ LFEALAEDDA | |
| | VCVWHKDGDA AKAPDPQPLI DYALAAEAEF KKRHFKVPAY RHPDALLHPI | |
| | FCDFGKSRWD ICFDVHKNMQ TPFPRALCLT LWTGSEMKRI PLCWQSKRLA | |
| | RDLALGNNTG DAGASEVTRA DRLGRAASRA ASNVTKSDVV NIAGLFEQAD | |
| | WNGRLQAPRQ QLEAIARYVE KHDWDQKAEK MRNAIQWLVT FSARLQPQGP | |
| | WCAYAKIHGL KEDPQYWPHA DTNKNRKGHA RLILSRLPGL RVLAVDLGHR | |
| | YAAACAVWEA LSTEAFQREI KGRTILRGRT DGNALYCHTR HKANGKERVT | |
| | IYRRIGADTL PDGKPHPAPW ARLDRQFLIK LQGEEEGVRE ASNEEIWAVH | |
| | QLEAALGRPV SLIDRLVASG WGGSDKQKAR LEGLKQLGWD PADKPSLSVD | |
| | ELMSSAVRTM RLALKRHGDR ARIAHYLITD EKTTPGGIKE TLDEKGRIDL | |
| | LQDALVLWHD LFSSRGWRDD TAKQLWNAHV AKLHGYKAPE EPGEDSSGAE | |
| | RKKKQRENRE KLYDVAKALA QDVTLREALH DAWKKRWEND DERWKKQLRW | |
| | FKDWVFPRGN HASDPTIRKR QLINPSGGNG RRGNHASDPT IRKRQLINPS | |
| | GGNGRRGNHA SDPTIRKVGG LSLPRLATLT EFRRKVQVGF FTRLKPDGTR | |
| | AETKEQFGQS ALDALEHLRE QRVKQLASRI AEAALGVGRV RRPVEGKDPK | |
| | RPDVRVDEPC HAIVIEDLTH YRPEETRTRR ENRQLMTWSS SKVKKYLAEA | |
| | CQLHGLHLRE VSASYTSRQD SRTGAPGVRC QDVPVKEFMR SPFWRKQVKQ | |
| | AEAKQAANKG DARERLLCDL NARWKDRTAA DWEKAGAVRI PLQGGEIFVS | |
| | ADANSPAAKG IQADLNAAAN IGLRALTDPD WAGKWWYVPC DPASFRPVRD | |
| | KVDGSAVVNP DQPLRQSAQA QSGDAAKDKN GNKGAGKSKE VVNLWRDISS | |
| | SPLECIEFGE WKEYAAYQNE VQCRVIRILK EQIKGRDKQP HEGSKEDDIP | |
| | L | |
| Desulfovibrioinopinatus | MPTRTINLKL VLGKNPENAT LRRALFSTHR LVNQATKRIE EFLLLCRGEA | (SEQ |
| WP_027186183.1 | YRTVDNEGKE AEIPRHAVQE EALAFAKAAQ RHNGCISTYE DQEILDVLRQ | ID |
| | LYERLVPSVN ENNEAGDAQA ANAWVSPLMS AESEGGLSVY DKVLDPPPVW | NO: |
| | MKLKEEKAPG WEAASQIWIQ SDEGQSLLNK PGSPPRWIRK LRSGQPWQDD | 195) |
| | FVSDQKKKQD ELTKGNAPLI KQLKEMGLLP LVNPFFRHLL DPEGKGVSPW | |
| | DRLAVRAAVA HFISWESWNH RTRAEYNSLK LRRDEFEAAS DEFKDDETLL | |
| | RQYEAKRHST LKSIALADDS NPYRIGVRSL RAWNRVREEW IDKGATEEQR | |
| | VTILSKLQTQ LRGKFGDPDL FNWLAQDRHV HLWSPRDSVT PLVRINAVDK | |
| | VLRRRKPYAL MTFAHPRFHP RWILYEAPGG SNLRQYALDC TENALHITLP | |
| | LLVDDAHGTW IEKKIRVPLA PSGQIQDLTL EKLEKKKNRL YYRSGFQQFA | |
| | GLAGGAEVLF HRPYMEHDER SEESLLERPG AVWEKLTLDV ATQAPPNWLD | |
| | GKGRVRTPPE VHHFKTALSN KSKHTRTLQP GLRVLSVDLG MRTFASCSVE | |
| | ELIEGKPETG RAFPVADERS MDSPNKLWAK HERSEKLTLP GETPSRKEEE | |
| | ERSIARAEIY ALKRDIQRLK SLLRLGEEDN DNRRDALLEQ FFKGWGEEDV | |
| | VPGQAFPRSL FQGLGAAPFR STPELWRQHC QTYYDKAEAC LAKHISDWRK | |
| | RTRPRPTSRE MWYKTRSYHG GKSIWMLEYL DAVRKLLLSW SLRGRTYGAI | |
| | NRQDTARFGS LASRLLHHIN SLKEDRIKTG ADSIVQAARG YIPLPHGKGW | |
| | EQRYEPCQLI LFEDLARYRF RVDRPRRENS QLMQWNHRAI VAETTMQAEL | |
| | YGQIVENTAA GESSRFHAAT GAPGVRCREL LERDEDNDLP KPYLLRELSW | |
| | MLGNTKVESE EEKLRLLSEK IRPGSLVPWD GGEQFATLHP KRQTLCVIHA | |
| | DMNAAQNLQR RFFGRCGEAF RLVCQPHGDD VLRLASTPGA RLLGALQQLE | |
| | NGQGAFELVR DMGSTSQMNR FVMKSLGKKK IKPLQDNNGD DELEDVLSVL | |
| | PEEDDTGRIT VERDSSGIFF PCNVWIPAKQ FWPAVRAMIW KVMASHSLG | |
| Desulfonatronum | MVLGRKDDTA ELRRALWITH EHVNLAVAEV ERVLLRCRGR SYWTLDRRGD | (SEQ |
| thiodismutans | PVHVPESQVA EDALAMAREA QRRNGWPVVG EDEEILLALR YLYEQIVPSC | ID |
| WP_031386437.1 | LLDDLGKPLK GDAQKIGTNY AGPLFDSDTC RRDEGKDVAC CGPFHEVAGK | NO: |
| | YLGALPEWAT PISKQEFDGK DASHLRFKAT GGDDAFFRVS IEKANAWYED | 187) |
| | PANQDALKNK AYNKDDWKKE KDKGISSWAV KYIQKQLQLG QDPRTEVRRK | |
| | LWLELGLLPL FIPVEDKTMV GNLWNRLAVR LALAHLLSWE SWNHRAVQDQ | |
| | ALARAKRDEL AALFLGMEDG FAGLREYELR RNESIKQHAF EPVDRPYVVS | |
| | GRALRSWTRV REEWLRHGDT QESRKNICNR LQDRLRGKFG DPDVFHWLAE | |
| | DGQEALWKER DCVTSFSLLN DADGLLEKRK GYALMTFADA RLHPRWAMYE | |
| | APGGSNLRTY QIRKTENGLW ADVVLLSPRN ESAAVEEKTE NVRLAPSGQL | |
| | SNVSFDQIQK GSKMVGRCRY QSANQQFEGL LGGAEILEDR KRIANEQHGA | |
| | TDLASKPGHV WFKLTLDVRP QAPQGWLDGK GRPALPPEAK HFKTALSNKS | |
| | KFADQVRPGL RVLSVDLGVR SFAACSVFEL VRGGPDQGTY FPAADGRTVD | |
| | DPEKLWAKHE RSFKITLPGE NPSRKEEIAR RAAMEELRSL NGDIRRLKAI | |
| | LRLSVLQEDD PRTEHLRLFM EAIVDDPAKS ALNAELFKGF GDDRERSTPD | |
| | LWKQHCHFFH DKAEKVVAER FSRWRTETRP KSSSWQDWRE RRGYAGGKSY | |
| | WAVTYLEAVR GLILRWNMRG RTYGEVNRQD KKQFGTVASA LLHHINQLKE | |
| | DRIKTGADMI IQAARGFVPR KNGAGWVQVH EPCRLILFED LARYRERTDR | |
| | SRRENSRLMR WSHREIVNEV GMQGELYGLH VDTTEAGESS RYLASSGAPG | |
| | VRCRHLVEED FHDGLPGMHL VGELDWLLPK DKDRTANEAR RLLGGMVRPG | |
| | MLVPWDGGEL FATLNAASQL HVIHADINAA QNLQRREWGR CGEAIRIVCN | |
| | QLSVDGSTRY EMAKAPKARL LGALQQLKNG DAPFHLTSIP NSQKPENSYV | |
| | MTPTNAGKKY RAGPGEKSSG EEDELALDIV EQAEELAQGR KTFFRDPSGV | |
| | FFAPDRWLPS EIYWSRIRRR IWQVTLERNS SGRQERAEMD EMPY | |
| Tuberibacillus | MATKSFILKM KTKNNPQLRL SLWKTHELEN FGVAYYMDLL SLFRQKDLYM | (SEQ |
| calidus | HNDEDPDHPV VLKKEEIQER LWMKVRETQQ KNGFHGEVSK DEVLETLRAL | ID |
| WP_027726362.1 | YEELVPSAVG KSGEANQISN KYLYPLTDPA SQSGKGTANS GRKPRWKKLK | NO: |
| | EAGDPSWKDA YEKWEKERQE DPKLKILAAL QSFGLIPLER PFTENDHKAV | 196) |
| | ISVKWMPKSK NQSVRKEDKD MENQAIEREL SWESWNEKVA EDYEKTVSIY | |
| | ESLQKELKGI STKAFEIMER VEKAYEAHLR EITFSNSTYR IGNRAIRGWT | |
| | EIVKKWMKLD PSAPQGNYLD VVKDYQRRHP RESGDEKLFE LLSRPENQAA | |
| | WREYPEFLPL YVKYRHAEQR MKTAKKQATF TLCDPIRHPL WVRYEERSGT | |
| | NLNKYRLIMN EKEKVVQFDR LICLNADGHY EEQEDVTVPL APSQQFDDQI | |
| | KFSSEDTGKG KHNFSYYHKG INYELKGTLG GARIQFDREH LLRRQGVKAG | |
| | NVGRIFLNVT LNIEPMQPFS RSGNLQTSVG KALKVYVDGY PKVVNFKPKE | |
| | LTEHIKESEK NTLTLGVESL PTGLRVMSVD LGQRQAAAIS IFEVVSEKPD | |
| | DNKLFYPVKD TDLFAVHRTS FNIKLPGEKR TERRMLEQQK RDQAIRDLSR | |
| | KLKFLKNVLN MQKLEKTDER EKRVNRWIKD REREEENPVY VQEFEMISKV | |
| | LYSPHSVWVD QLKSIHRKLE EQLGKEISKW RQSISQGRQG VYGISLKNIE | |
| | DIEKTRRLLF RWSMRPENPG EVKQLQPGER FAIDQQNHLN HLKDDRIKKL | |
| | ANQIVMTALG YRYDGKRKKW IAKHPACQLV LFEDLSRYAF YDERSRLENR | |
| | NLMRWSRREI PKQVAQIGGL YGLLVGEVGA QYSSRFHAKS GAPGIRCRVV | |
| | KEHELYITEG GQKVRNQKFL DSLVENNIIE PDDARRLEPG DLIRDQGGDK | |
| | FATLDERGEL VITHADINAA QNLQKREWTR THGLYRIRCE SREIKDAVVL | |
| | VPSDKDQKEK MENLFGIGYL QPFKQENDVY KWVKGEKIKG KKTSSQSDDK | |
| | ELVSEILQEA SVMADELKGN RKTLERDPSG YVEPKDRWYT GGRYFGTLEH | |
| | LLKRKLAERR LEDGGSSRRG LENGTDSNTN VE | |
| Bacillus | MATRSFILKI EPNEEVKKGL WKTHEVLNHG IAYYMNILKL IRQEAIYEHH | (SEQ |
| thermoamylovorans | EQDPKNPKKV SKAEIQAELW DFVLKMQKCN SFTHEVDKDV VENILRELYE | ID |
| WP_041902512.1 | ELVPSSVEKK GEANQLSNKF LYPLVDPNSQ SGKGTASSGR KPRWYNLKIA | NO: |
| | GDPSWEEEKK KWEEDKKKDP LAKILGKLAE YGLIPLFIPF TDSNEPIVKE | 197) |
| | IKWMEKSRNQ SVRRLDKDMF IQALERFLSW ESWNLKVKEE YEKVEKEHKT | |
| | LEERIKEDIQ AFKSLEQYEK ERQEQLLRDT LNTNEYRLSK RGLRGWREII | |
| | QKWLKMDENE PSEKYLEVEK DYQRKHPREA GDYSVYEFLS KKENHFIWRN | |
| | HPEYPYLYAT FCEIDKKKKD AKQQATFTLA DPINHPLWVR FEERSGSNLN | |
| | KYRILTEQLH TEKLKKKLTV QLDRLIYPTE SGGWEEKGKV DIVLLPSRQF | |
| | YNQIFLDIEE KGKHAFTYKD ESIKFPLKGT LGGARVQFDR DHLRRYPHKV | |
| | ESGNVGRIYF NMTVNIEPTE SPVSKSLKIH RDDEPKFVNF KPKELTEWIK | |
| | DSKGKKLKSG IESLEIGLRV MSIDLGQRQA AAASIFEVVD QKPDIEGKLE | |
| | FPIKGTELYA VHRASFNIKL PGETLVKSRE VLRKAREDNL KLMNQKLNEL | |
| | RNVLHFQQFE DITEREKRVT KWISRQENSD VPLVYQDELI QIRELMYKPY | |
| | KDWVAFLKQL HKRLEVEIGK EVKHWRKSLS DGRKGLYGIS LKNIDEIDRT | |
| | RKFLLRWSLR PTEPGEVRRL EPGQRFAIDQ LNHLNALKED RLKKMANTII | |
| | MHALGYCYDV RKKKWQAKNP ACQIILFEDL SNYNPYEERS RFENSKLMKW | |
| | SRREIPRQVA LQGEIYGLQV GEVGAQFSSR FHAKTGSPGI RCSVVTKEKL | |
| | QDNRFFKNLQ REGRLTLDKI AVLKEGDLYP DKGGEKFISL SKDRKLVTTH | |
| | ADINAAQNLQ KRFWTRTHGF YKVYCKAYQV DGQTVYIPES KDQKQKIIEE | |
| | FGEGYFILKD GVYEWGNAGK LKIKKGSSKQ SSSELVDSDI LKDSEDLASE | |
| | DDSSKQSM DPSGNVFPSD KWMAAGVFFG KLERILISKL TNQYSISTIE | |
| | LKGEKLMLYR | |
| Bacillus sp. | MAIRSIKLKL KTHTGPEAQN LRKGIWRTHR LLNEGVAYYM KMLLLERQES | (SEQ |
| NSP2.1 | TGERPKEELQ EELICHIREQ QQRNQADKNT QALPLDKALE ALRQLYELLV | ID |
| WP_026557978.1 | PSSVGQSGDA QIISRKFLSP LVDPNSEGGK GTSKAGAKPT WQKKKEANDP | NO: |
| | TWEQDYEKWK KRREEDPTAS VITTLEEYGI RPIFPLYTNT VTDIAWLPLQ | 198) |
| | SNQFVRTWDR DMLQQAIERL LSWESWNKRV QEEYAKLKEK MAQLNEQLEG | |
| | GQEWISLLEQ YEENRERELR ENMTAANDKY RITKRQMKGW NELYELWSTE | |
| | PASASHEQYK EALKRVQQRL RGREGDAHFF QYLMEEKNRL IWKGNPQRIH | |
| | YFVARNELTK RLEEAKQSAT MTLPNARKHP LWVREDARGG NLQDYYLTAE | |
| | ADKPRSRRFV TFSQLIWPSE SGWMEKKDVE VELALSRQFY QQVKLLKNDK | |
| | GKQKIEFKDK GSGSTENGHL GGAKLQLERG DLEKEEKNFE DGEIGSVYLN | |
| | VVIDFEPLQE VKNGRVQAPY GQVLQLIRRP NEFPKVTTYK SEQLVEWIKA | |
| | SPQHSAGVES LASGERVMSI DLGLRAAAAT SIFSVEESSD KNAADFSYWI | |
| | EGTPLVAVHQ RSYMLRLPGE QVEKQVMEKR DERFQLHQRV KFQIRVLAQI | |
| | MRMANKQYGD RWDELDSLKQ AVEQKKSPLD QTDRTFWEGI VCDLTKVLPR | |
| | NEADWEQAVV QIHRKAEEYV GKAVQAWRKR FAADERKGIA GLSMWNIEEL | |
| | EGLRKLLISW SRRTRNPQEV NRFERGHTSH QRLLTHIQNV KEDRLKQLSH | |
| | AIVMTALGYV YDERKQEWCA EYPACQVILF ENLSQYRSNL DRSTKENSTL | |
| | MKWAHRSIPK YVHMQAEPYG IQIGDVRAEY SSRFYAKTGT PGIRCKKVRG | |
| | QDLQGRRFEN LQKRLVNEQF LTEEQVKQLR PGDIVPDDSG ELEMTLTDGS | |
| | GSKEVVELQA DINAAHNLQK REWQRYNELF KVSCRVIVRD EEEYLVPKTK | |
| | SVQAKLGKGL FVKKSDTAWK DVYVWDSQAK LKGKTTFTEE SESPEQLEDE | |
| | QEIIEEAEEA KGTYRTLERD PSGVFFPESV WYPQKDEWGE VKRKLYGKLR | |
| | ERELTKAR | |
| Alicyclobacillus | MAVKSIKVKL RLDDMPEIRA GLWKLHKEVN AGVRYYTEWL SLLRQENLYR | (SEQ |
| acidoterrestris | RSPNGDGEQE CDKTAEECKA ELLERLRARQ VENGHRGPAG SDDELLQLAR | ID |
| WP_021296342.1 | QLYELLVPQA IGAKGDAQQI ARKELSPLAD KDAVGGLGIA KAGNKPRWVR | NO: |
| | MREAGEPGWE EEKEKAETRK SADRTADVLR ALADFGLKPL MRVYTDSEMS | 199) |
| | SVEWKPLRKG QAVRTWDRDM FQQAIERMMS WESWNQRVGQ EYAKLVEQKN | |
| | RFEQKNFVGQ EHLVHLVNQL QQDMKEASPG LESKEQTAHY VTGRALRGSD | |
| | KVFEKWGKLA PDAPFDLYDA EIKNVQRRNT RRFGSHDLFA KLAEPEYQAL | |
| | WREDASFLTR YAVYNSILRK LNHAKMFATF TLPDATAHPI WTREDKLGGN | |
| | LHQYTFLENE FGERRHAIRF HKLLKVENGV AREVDDVTVP ISMSEQLDNL | |
| | LPRDPNEPIA LYFRDYGAEQ HETGEFGGAK IQCRRDQLAH MHRRRGARDV | |
| | YLNVSVRVQS QSEARGERRP PYAAVFRLVG DNHRAFVHED KLSDYLAEHP | |
| | DDGKLGSEGL LSGLRVMSVD LGLRTSASIS VERVARKDEL KPNSKGRVPF | |
| | FFPIKGNDNL VAVHERSQLL KLPGETESKD LRAIREERQR TLRQLRTQLA | |
| | YLRLLVRCGS EDVGRRERSW AKLIEQPVDA ANHMTPDWRE AFENELQKLK | |
| | SLHGICSDKE WMDAVYESVR RVWRHMGKQV RDWRKDVRSG ERPKIRGYAK | |
| | DVVGGNSIEQ IEYLERQYKF LKSWSFFGKV SGQVIRAEKG SRFAITLREH | |
| | IDHAKEDRLK KLADRIIMEA LGYVYALDER GKGKWVAKYP PCQLILLEEL | |
| | SEYQFNNDRP PSENNQLMQW SHRGVFQELI NQAQVHDLLV GTMYAAFSSR | |
| | FDARTGAPGI RCRRVPARCT QEHNPEPFPW WINKFVVEHT LDACPLRADD | |
| | LIPTGEGEIF VSPESAEEGD FHQIHADLNA AQNLQQRLWS DEDISQIRLR | |
| | CDWGEVDGEL VLIPRLTGKR TADSYSNKVF YTNTGVTYYE RERGKKRRKV | |
| | FAQEKLSEEE AELLVEADEA REKSVVLMRD PSGIINRGNW TRQKEFWSMV | |
| | NQRIEGYLVK QIRSRVPLQD SACENTGDI | |
| Alicyclobacillus | MTVRSIRVKL AVGSPQYRDV RRGLWKTHEI MNQGVRYYCE WLVLMRQEPI | (SEQ |
| hesperidum | YDEDEHGLTV VQRTREDIQA ELLSRLRTLQ SAHQHSGDMG TDEELLSLMR | ID |
| WP_074693942.1 | QLYEQLVPSS VDKNKSGDAR MIARNFENPL TNPNSQGGLG ISNAGRKPKW | NO: |
| | LLKKLSGDPT WEEDYKKAME QKQESSVSFL LLELRRFGLH PIFLPYTDTV | 200) |
| | LEVSWAPKKA RQWVRKWDYD LFQQSIERML SWESWTRRVK ERFEKLVESE | |
| | KKFYDENFAT DPEFIKLAET LEGELQASSQ GFVAVDEHAF QIRPRSMRGF | |
| | DRVADEWCKL ADDAPIEEYE AAIKRVQARL GRNFGSYVLF AHLAKPEYWS | |
| | LWRSDPTKIL RFARLRALQR AVARAKRHAR LTLPDAIHHP IWIRYDAKGK | |
| | NIYSYRLLIP EKRSKRYYVE FSSLIMPDGE NRWAEHRNIR VPLAFSRQWE | |
| | RLHFSIMEDG SLCVQYRDPG VDEPLRAELG GAKIQFDRRY LIRRSSTLSA | |
| | GECGPVYLNV SVDVNPAHRP DVQVLQSAKL VSVSRDTNRI YLRPENLSAY | |
| | WKSQGDGTLP LRVMSVDLGV RSSAAVVICR LEHRDSVVSS GRRTATIYRI | |
| | AGTDEFVAVQ ERAFLLRLPG EGKGTNEDAP LRDVYAQLGT IRQGIQILRS | |
| | LLRLCDTKTP DERQEALHGL AQSLEPSGAW KDELHPHLVM LQGVVHDSVD | |
| | NWKQKVISVH RQMERILGHA VREWKVARKN AGKPPIRRGA GGLSLRRIRQ | |
| | LEQERRTLVA WSNHAREPGQ VVRIKRGTQV AQWLVERVNH LKEDRLKKLA | |
| | DLLIMTALGY VYDETKPSGH KWDKRYPPCQ IILMEDLSRY RFQSDRPPSE | |
| | NSQLMAWSHR RLLEILKLQA DLHKLIVGTV FPAFSSREDA QSGAPGVRCR | |
| | SVKKQDIENA AQGKGWLARE LQRLNWTLEW LQPNDLIPTG DGELFVTPAC | |
| | CDRQKGIKIV HADLNAAQNL QRRFWGGHAE SLCRVTCDVV ERDGRRYAVP | |
| | RISNAFADSF YKVFGQGVFV STDEEDVYRW MVGEKISSRG RSRGRTSDEE | |
| | AEAETWIDEA REQQGKVIAL FRDASGQIHG GDWLVAKVEW GWVERLVTAR | |
| | LLSRMSEREA AAHKE | |
| Alicyclobacillus | MAVKSMKVKL RLDNMPEIRA GLWKLHTEVN AGVRYYTEWL SLLRQENLYR | (SEQ |
| acidiphilus | RSPNGDGEQE CYKTAEECKA ELLERLRARQ VENGHCGPAG SDDELLQLAR | ID |
| WP_067623834.1 | QLYELLVPQA IGAKGDAQQI ARKELSPLAD KDAVGGLGIA KAGNKPRWVR | NO: |
| | MREAGEPGWE EEKAKAEARK STDRTADVLR ALADFGLKPL MRVYTDSDMS | 201) |
| | SVQWKPLRKG QAVRTWDRDM FQQAIERMMS WESWNQRVGE AYAKLVEQKS | |
| | RFEQKNFVGQ EHLVQLVNQL QQDMKEASHG LESKEQTAHY LTGRALRGSD | |
| | KVFEKWEKLD PDAPFDLYDT EIKNVQRRNT RRFGSHDLFA KLAEPKYQAL | |
| | WREDASELTR YAVYNSIVRK LNHAKMFATF TLPDATAHPI WTREDKLGGN | |
| | LHQYTFLENE FGEGRHAIRF QKLLTVEDGV AKEVDDVTVP ISMSAQLDDL | |
| | LPRDPHELVA LYFQDYGAEQ HLAGEFGGAK IQYRRDQLNH LHARRGARDV | |
| | YLNLSVRVQS QSEARGERRP PYAAVERLVG DNHRAFVHED KLSDYLAEHP | |
| | DDGKLGSEGL LSGLRVMSVD LGLRTSASIS VERVARKDEL KPNSEGRVPF | |
| | CFPIEGNENL VAVHERSQLL KLPGETESKD LRAIREERQR TLRQLRTQLA | |
| | YLRLLVRCGS EDVGRRERSW AKLIEQPMDA NQMTPDWREA FEDELQKLKS | |
| | LYGICGDREW TEAVYESVRR VWRHMGKQVR DWRKDVRSGE RPKIRGYQKD | |
| | VVGGNSIEQI EYLERQYKFL KSWSFFGKVS GQVIRAEKGS RFAITLREHI | |
| | DHAKEDRLKK LADRIIMEAL GYVYALDDER GKGKWVAKYP PCQLILLEEL | |
| | SEYQENNDRP PSENNQLMQW SHRGVFQELL NQAQVHDLLV GTMYAAFSSR | |
| | FDARTGAPGI RCRRVPARCA REQNPEPFPW WINKFVAEHK LDGCPLRADD | |
| | LIPTGEGEFF VSPESAEEGD FHQIHADLNA AQNLQRRLWS DEDISQIRLR | |
| | CDWGEVDGEP VLIPRTTGKR TADSYGNKVF YTKTGVTYYE RERGKKRRKV | |
| | FAQEELSEEE AELLVEADEA REKSVVLMRD PSGIINRGDW TRQKEFWSMV | |
| | NQRIEGYLVK QIRSRVRLQE SACENTGDI | |
| Alicyclobacillus | MAVKSIKVKL MLGHLPEIRE GLWHLHEAVN LGVRYYTEWL ALLRQGNLYR | (SEQ |
| macrosporangiidus | RGKDGAQECY MTAEQCRQEL LVRLRDRQKR NGHTGDPGTD EELLGVARRL | ID |
| SFU30094.1 | YELLVPQSVG KKGQAQMLAS GELSPLADPK SEGGKGTSKS GRKPAWMGMK | NO: |
| | EAGDSRWVEA KARYEANKAK DPTKQVIASL EMYGLRPLED VFTETYKTIR | 202) |
| | WMPLGKHQGV RAWDRDMFQQ SLERLMSWES WNERVGAEFA RLVDRRDRER | |
| | EKHFTGQEHL VALAQRLEQE MKEASPGFES KSSQAHRITK RALRGADGII | |
| | DDWLKLSEGE PVDREDEILR KRQAQNPRRF GSHDLFLKLA EPVFQPLWRE | |
| | DPSFLSRWAS YNEVLNKLED AKQFATFTLP SPCSNPVWAR FENAEGTNIF | |
| | KYDFLFDHFG KGRHGVRFQR MIVMRDGVPT EVEGIVVPIA PSRQLDALAP | |
| | NDAASPIDVF VGDPAAPGAF RGQFGGAKIQ YRRSALVRKG RREEKAYLCG | |
| | FRLPSQRRTG TPADDAGEVF LNLSLRVESQ SEQAGRRNPP YAAVFHISDQ | |
| | TRRVIVRYGE IERYLAEHPD TGIPGSRGLT SGLRVMSVDL GLRTSAAISV | |
| | FRVAHRDELT PDAHGRQPFF FPIHGMDHLV ALHERSHLIR LPGETESKKV | |
| | RSIREQRLDR LNRLRSQMAS LRLLVRTGVL DEQKRDRNWE RLQSSMERGG | |
| | ERMPSDWWDL FQAQVRYLAQ HRDASGEAWG RMVQAAVRTL WRQLAKQVRD | |
| | WRKEVRRNAD KVKIRGIARD VPGGHSLAQL DYLERQYRFL RSWSAFSVQA | |
| | GQVVRAERDS REAVALREHI DNGKKDRLKK LADRILMEAL GYVYVTDGRR | |
| | AGQWQAVYPP CQLVLLEELS EYRESNDRPP SENSQLMVWS HRGVLEELIH | |
| | QAQVHDVLVG TIPAAFSSRF DARTGAPGIR CRRVPSIPLK DAPSIPIWLS | |
| | HYLKQTERDA AALRPGELIP TGDGEFLVTP AGRGASGVRV VHADINAAHN | |
| | LQRRLWENED LSDIRVRCDR REGKDGTVVL IPRLTNQRVK ERYSGVIFTS | |
| | EDGVSFTVGD AKTRRRSSAS QGEGDDLSDE EQELLAEADD ARERSVVLFR | |
| | DPSGFVNGGR WTAQRAFWGM VHNRIETLLA ERFSVSGAAE KVRG | |
| Sulfobacillus | RQSREDASPQ IIISASDLKA DLLYHARQQQ KEHVPRITGS DAEVLGALRQ | (SEQ |
| thermosulfidooxidans | VYELIVPSSV GKSGDSKTIA RKELSPLTDP DSAGGRDQSA SGRKPTWTKM | ID |
| PSR34340.1 | KAEGNPLWEE KERQWKDRKD NDPTPFVLNQ LADYGLLPLI RLFTDVGENI | NO: |
| | FDPKKPGQFV RPWDRSMFQQ AIERLMSWES WNQRVRQEWE ALTQKHSAFY | 203) |
| | REQFTAEPDA ALYRVAQSLE EEMRKEHQGF ATDAPEAFRI RRVALKGEDR | |
| | LLERWQKTLG KNGQSATLLD DIRRVQSDLG DKFGSAPLYQ KLVDERWQRL | |
| | WTVDPTFLQR YAAFNDLTQR LQRAKRVANL TLPDAVAHPI WSRYEGPNAS | |
| | SGNRYHIHLP TTGQPSSVTF DRILWPDGDG GWYERKRVTV FLRPSHQVDR | |
| | IREAPTDSVV DNFPLVVEDQ SARTILRASW GGAKLEYDRN RLPRQLKKGV | |
| | PDSIYLSLTL NLDTTKPSGL FHMQQNGRVW IRKDVVMQYY NEIPGDNVQF | |
| | KPLYVMSVDL GIRSAAAVSI FSVQLKTGIE EHRLTYPVAD CPGLVAVHER | |
| | SVLLTMPGER REQRDRRYEQ QRQGLRELRT DMRGMNDLLR GAYVDGDRRE | |
| | EFLARLSKLE ETSPELWEPV YRSLNDSKMA PAAEWERLVV YCHRQVEQSL | |
| | SSRIQNLRSG RSAYRMSGGL SLDHVQDLER IRGIIASWTN HPRIPGSVVR | |
| | WQQGRSHTVA LGRHILELKR DRVKKVANYL IMTALGYAYD SKRARGEKWV | |
| | RRYPSCHLMV FEDLTRYRER TDRPRSENRQ LMRWTHQELI AVTGIQAEPH | |
| | GILVGTMYAG FSSRFDAVTK APGVRGATVR QILRTRGMVR LKEIAADVGV | |
| | DINTLRPHDV LPTGDGEYLL SVVRHRDSYR LKQVHADINA AHNLQRRLWT | |
| | QDEVERVSCR LALNSERVVA TPPPSYNKRY GKGFFEKGDN GVYIWKTGGK | |
| | IKISDMLEED MDIPEDTAEL LRGNSVTLER DPSGTIAGGN WLEAKEFWGR | |
| | VNSLVNKGVR DKILGGIPVD NSSAHAE | |
| Spirochaeta sp. | MGLLLPSLSR TVNVTIHLIL HPRKKGSRHR EYAVMLDHAV RKIFLAHNWI | (SEQ |
| LUC14_002_19_P3 | KRAEAERQKF EADLYKIDRV PQEARDWLDE FCRERTESTG SIDGYHIRRK | ID |
| OQX29950.1 | AVLGWEALVE AWDQKDCLSV EDRIAAARDL QDNPGMDKFG DIWLYEALAS | NO: |
| | APCVWQKDGE PNAQILLDYV DAGEAEYKRS HYKVPAYRHP DPLLHPIFCD | 204) |
| | FGQSRWSISF DIHEFKKNGE KNPVNIHALT MGLVSKKRIV KTELKWSSKR | |
| | LNSNLALSLE SPEDAIEVSR ATRLGRAAVG ASQDRAVNIA GLFESAGWNG | |
| | RLQAPRKQLE ALAKLEEDKS AEALAKALRN RIKWFITFSP KLQPHGPWME | |
| | YAERFSGEAP SRAAVIKGKY TVIHQDKTRR RPLAKLHLCR MPGLRVLSVD | |
| | LGHRHAAACA VWETLSSESM EKKCREAGCL PPAPEDLYLH LKKKNKTAVY | |
| | RRIGGNFLPD GNEHPAPWAK LDRQFIIDLQ GEEGCTRMAL AGEIWQVHCM | |
| | EKVFGRSIPL VDRLVRAGWG EKNKQPEILQ ELKQKGWVPL EVSKTNTGYH | |
| | YSLCVDSLMT LAVNTVRFAL RRHACRARIA YYMEGGAIPE GGLPENSGNK | |
| | DFIVEALMLW YELATDSRWN GSWEANFWDE NEDKKLAEIQ DAVNEREGDK | |
| | AKIIKQKERK ELLKKEFIPL AEGLLENSRR ISIASQWRMV WNEEDAIWQS | |
| | ELRSLRDWIL PKGTRGKKRT IRHVGGLSLS RLAVIKSLYR VQKSFYTRMK | |
| | PEGEPMDGTM AVGEGFGQKI LDDLETMKEQ RVKQLASRVV EAALGTGRIK | |
| | KPENNKTPKR PFTAVDEPCH AVVIENLTHY RPENKRTRRE NRQLMTWSSS | |
| | KVKKYLFESC QLHGLYLFEV QASYTSRQDS RTGAPGVRCS ELSVKKFLES | |
| | PFRQREIAHA EENMAQENPC NRYLIALHNK WKNREYDKTA PPLRIPHWGG | |
| | EIFVSALTGN TLQADLNAAA NIGLQALLDP DWPGRWWYVP AVKGCDGRRI | |
| | PHSKCSGAAC LDNWRVGLKN NLYTGVRTPL PGKNKGSTSG EDVHKSNAVE | |
| | KSTINLWRDI SVLPLTEGQW | |
| Bacillus hisashii | MATRSFILKI EPNEEVKKGL WKTHEVLNHG IAYYMNILKL IRQEAIYEHH | (SEQ |
| strain C4 v4 | EQDPKNPKKV SKAEIQAELW DFVLKMQKCN SFTHEVDKDE VENILRELYE | ID |
| mutant of | ELVPSSVEKK GEANQLSNKF LYPLVDPNSQ SGKGTASSGR KPRWYNLKIA | NO: |
| WP_095142515.1 | GDPSWEEEKK KWEEDKKKDP LAKILGKLAE YGLIPLFIPY TDSNEPIVKE | 205) |
| K846R | IKWMEKSRNQ SVRRLDKDMF IQALERFLSW ESWNLKVKEE YEKVEKEYKT | |
| S893R | LEERIKEDIQ ALKALEQYEK ERQEQLLRDT LNTNEYRLSK RGLRGWREII | |
| E837G | QKWLKMDENE PSEKYLEVEK DYQRKHPREA GDYSVYEFLS KKENHFIWRN | |
| | HPEYPYLYAT FCEIDKKKKD AKQQATFTLA DPINHPLWVR FEERSGSNLN | |
| | KYRILTEQLH TEKLKKKLTV QLDRLIYPTE SGGWEEKGKV DIVLLPSRQF | |
| | YNQIFLDIEE KGKHAFTYKD ESIKFPLKGT LGGARVQFDR DHLRRYPHKV | |
| | ESGNVGRIYF NMTVNIEPTE SPVSKSLKIH RDDEPKVVNF KPKELTEWIK | |
| | DSKGKKLKSG IESLEIGLRV MSIDLGQRQA AAASIFEVVD QKPDIEGKLE | |
| | FPIKGTELYA VHRASENIKL PGETLVKSRE VLRKAREDNL KLMNQKLNEL | |
| | RNVLHFQQFE DITEREKRVT KWISRQENSD VPLVYQDELI QIRELMYKPY | |
| | KDWVAFLKQL HKRLEVEIGK EVKHWRKSLS DGRKGLYGIS LKNIDEIDRT | |
| | RKFLLRWSLR PTEPGEVRRL EPGQRFAIDQ LNHLNALKED RLKKMANTII | |
| | MHALGYCYDV RKKKWQAKNP ACQIILFEDL SNYNPYGERS RFENSRLMKW | |
| | SRREIPRQVA LQGEIYGLQV GEVGAQFSSR FHAKTGSPGI RCRVVTKEKL | |
| | QDNRFFKNLQ REGRLTLDKI AVLKEGDLYP DKGGEKFISL SKDRKCVTTH | |
| | ADINAAQNLQ KREWTRTHGF YKVYCKAYQV DGQTVYIPES KDQKQKIIEE | |
| | FGEGYFILKD GVYEWVNAGK LKIKKGSSKQ SSSELVDSDI LKDSEDLASE | |
| | LKGEKLMLYR DPSGNVEPSD KWMAAGVFFG KLERILISKL TNQYSISTIE | |
| | DDSSKQSM | |

TABLE 7
Cas12c (C2c3) orthologs

| | | |
|---|---|---|
| OspCas12c | MTKLRHRQKK LTHDWAGSKK REVLGSNGKL QNPLLMPVKK GQVTEFRKAF | (SEQ ID |
| AWU30132.1 | SAYARATKGE MTDGRKNMFT HSFEPFKTKP SLHQCELADK AYQSLHSYLP | NO: 206) |
| KZX85786.1 | GSLAHFLLSA HALGFRIFSK SGEATAFQAS SKIEAYESKL ASELACVDLS | |
| | IQNLTISTLF NALTTSVRGK GEETSADPLI ARFYTLLTGK PLSRDTQGPE | |
| | RDLAEVISRK IASSFGTWKE MTANPLQSLQ FFEEELHALD ANVSLSPAFD | |
| | VLIKMNDLQG DLKNRTIVFD PDAPVFEYNA EDPADIIIKL TARYAKEAVI | |
| | KNQNVGNYVK NAITTTNANG LGWLLNKGLS LLPVSTDDEL LEFIGVERSH | |
| | PSCHALIELI AQLEAPELFE KNVFSDTRSE VQGMIDSAVS NHIARLSSSR | |
| | NSLSMDSEEL ERLIKSFQIH TPHCSLFIGA QSLSQQLESL PEALQSGVNS | |
| | ADILLGSTQY MLTNSLVEES IATYQRTLNR INYLSGVAGQ INGAIKRKAI | |
| | DGEKIHLPAA WSELISLPFI GQPVIDVESD LAHLKNQYQT LSNEFDTLIS | |
| | ALQKNFDLNF NKALLNRTQH FEAMCRSTKK NALSKPEIVS YRDLLARLTS | |
| | CLYRGSLVLR RAGIEVLKKH KIFESNSELR EHVHERKHFV FVSPLDRKAK | |
| | KLLRLTDSRP DLLHVIDEIL QHDNLENKDR ESLWLVRSGY LLAGLPDQLS | |
| | SSFINLPIIT QKGDRRLIDL IQYDQINRDA FVMLVTSAFK SNLSGLQYRA | |
| | NKQSFVVTRT LSPYLGSKLV YVPKDKDWLV PSQMFEGRFA DILQSDYMVW | |
| | KDAGRLCVID TAKHLSNIKK SVFSSEEVLA FLRELPHRTF IQTEVRGLGV | |
| | NVDGIAFNNG DIPSLKTFSN CVQVKVSRTN TSLVQTLNRW FEGGKVSPPS | |
| | IQFERAYYKK DDQIHEDAAK RKIRFQMPAT ELVHASDDAG WTPSYLLGID | |
| | PGEYGMGLSL VSINNGEVLD SGFIHINSLI NFASKKSNHQ TKVVPRQQYK | |
| | SPYANYLEQS KDSAAGDIAH ILDRLIYKLN ALPVFEALSG NSQSAADQVW | |
| | TKVLSFYTWG DNDAQNSIRK QHWFGASHWD IKGMLRQPPT EKKPKPYIAF | |
| | PGSQVSSYGN SQRCSCCGRN PIEQLREMAK DTSIKELKIR NSEIQLFDGT | |
| | IKLFNPDPST VIERRRHNLG PSRIPVADRT FKNISPSSLE FKELITIVSR | |
| | SIRHSPEFIA KKRGIGSEYF CAYSDCNSSL NSEANAAANV AQKFQKQLFF | |
| | EL | |
| QFN42172.1 | MRSNYHGGRN ARQWRKQISG LARRTKETVF TYKFPLETDA AEIDFDKAVQ | (SEQ ID |
| | TYGIAEGVGH GSLIGLVCAF HLSGFRLFSK AGEAMAFRNR SRYPTDAFAE | NO: 207) |
| | KLSAIMGIQL PTLSPEGLDL IFQSPPRSRD GIAPVWSENE VRNRLYTNWT | |
| | GRGPANKPDE HLLEIAGEIA KQVFPKFGGW DDLASDPDKA LAAADKYFQS | |
| | QGDFPSIASL PAAIMLSPAN STVDFEGDYI AIDPAAETLL HQAVSRCAAR | |
| | LGRERPDLDQ NKGPFVSSLQ DALVSSQNNG LSWLFGVGFQ HWKEKSPKEL | |
| | IDEYKVPADQ HGAVTQVKSF VDAIPLNPLF DTTHYGEFRA SVAGKVRSWV | |
| | ANYWKRLLDL KSLLATTEFT LPESISDPKA VSLFSGLLVD PQGLKKVADS | |
| | LPARLVSAEE AIDRLMGVGI PTAADIAQVE RVADEIGAFI GQVQQFNNQV | |
| | KQKLENLQDA DDEEFLKGLK IELPSGDKEP PAINRISGGA PDAAAEISEL | |
| | EEKLQRLLDA RSEHFQTISE WAEENAVTLD PIAAMVELER LRLAERGATG | |
| | DPEEYALRLL LQRIGRLANR VSPVSAGSIR ELLKPVFMEE REFNLFFHNR | |
| | LGSLYRSPYS TSRHQPFSID VGKAKAIDWI AGLDQISSDI EKALSGAGEA | |
| | LGDQLRDWIN LAGFAISQRL RGLPDTVPNA LAQVRCPDDV RIPPLLAMLL | |
| | EEDDIARDVC LKAFNLYVSA INGCLFGALR EGFIVRTRFQ RIGTDQIHYV | |
| | PKDKAWEYPD RLNTAKGPIN AAVSSDWIEK DGAVIKPVET VRNLSSTGFA | |
| | GAGVSEYLVQ APHDWYTPLD LRDVAHLVTG LPVEKNITKL KRLTNRTAFR | |
| | MVGASSFKTH LDSVLLSDKI KLGDFTIIID QHYRQSVTYG GKVKISYEPE | |
| | RLQVEAAVPV VDTRDRTVPE PDTLFDHIVA IDLGERSVGF AVFDIKSCLR | |
| | TGEVKPIHDN NGNPVVGTVA VPSIRRLMKA VRSHRRRRQP NQKVNQTYST | |
| | ALQNYRENVI GDVCNRIDTL MERYNAFPVL EFQIKNFQAG AKQLEIVYGS | |
| QFN42158.1 | MKKFELKQNF RNNYSGKTLR NFRQTLAQIA NKKSSDSILT IKFKLDCSKT | (SEQ ID |
| | GKLPKYENLI SLYDTIEDIK KGTLSYYLFT LIVSGFKFFG SASQAKAFST | NO: 208) |
| | KDIFKDNDFY NQFKIQSHLD LPDFVPSKIY QRLKKNVRST NGKDNAFKAS | |
| | VIVAEYRKEI GKLKNKDESS EHQCEELFKK IGTALETRFS SWQDLINNCS | |
| | TGCEIIDEIL NDSFGTLPSI KKMVLASTTQ SSDGEQDGIA IAYDPDSTFI | |
| | KSDELLNPYF AVATILKSMP PEIQQDKKSA YVKANLTTPT HNALSWIFGK | |
| | GLTLFQTEST EKLCAMFNVS DKRVIEQVQD AAKAVKLPAE LDLNHCTLKF | |
| | QDFRSSLGGH LDSWTTNYLK RLDELNDLLL NLPKNLSLPD IFMIDGKDFI | |
| | EYSGCNRDEI QQMIDFVVNE QNRIKLQESL NALLGKGNNQ ICSDDISTVK | |
| | DFSEIVNSLH SFVQQIDNSL EQSSNEANSI FSELKKKIEK NEKWDIWKNN | |
| | LKKIPKLNKL SGGVPDAWKE IREIEQKFHE ISENQKKHFT EVMEWIDAGN | |
| | GTIDIFESRF KYDELLKKSK KNNLQSADEL AFRSVLNKLG RFARQGNDLV | |
| | CEKIKNWFKE QNIFDSSKDF NRYFINQKGF IFKHPSSKKD NSPYNLSANL | |
| | LEKRYEVTNT VGALLEQCES DPAIVNDPFS MRSLVEFRAL WFSINISGIS | |
| | KEQHIPTKIA QPKLDDSTYQ ESVSPTLKYR LEKEQITSSE LNSIFTVYKS | |
| | LLSGLSIRLS RNSFYLRTKF SWIGNNSLIY CPKETTWKIP AAYFKSDLWN | |
| | EYKDKQILIV NEEYDVDVVK TFESVYKIVK SKDNNEKNRI LPLLKQLPHD | |
| | WMFKLPFGAS NAEKCKVLKL EKNNKKFKPL SVSKDSLARL SGPSTYFNQI | |
| | DEIMMNDESE LSEMTLLADE PVRQQMSNGK IEIIPDDYVM SLAIPITRSL | |
| | KKGNTESFPF KNIVSIDQGE AGFAYAVFKL SDCGNERAEP IATGLIPIPS | |
| | IRRLIHSVKK YRGKKQRIQN FNQKFDSTMF TLRENVTGDI CGLIVALMKK | |
| | YNAFPILEKQ VGNLESGSKQ LMLVYKAVNS KFLAAKVDMQ NDQRRSWWYQ | |
| | GNSWNTPILR ISNPNQSNNK NIVKNINGKK YEELKIYPGY SVSAYMTSCI | |
| | CHVCGRNALE LLKNDDSTGK VKKYQINQDG EVTIGGEVIK LYRKPDRLTP | |
| | VKNLAKKGNR ERTYASINER APMSKDTTQS RYFCVFKNCP CHNKEQHADV | |
| | NAAINIGRRF LKDCILDDNK EKD | |
| QFN42173.1 | MNARDWRKHV GVLAQQHKET TRTYTFPLDT TGSAIDFDAA LQAYNAVEGV | (SEQ ID |
| | GYGSLLGLAC AVHLSGFRLF STGKEAATER NRARYPNAAF QAALRKELGT | NO: 209) |
| | TITTLTPETL DRLFSSRPKR RNGVPLPWNQ DSIRDRLYTN WVKPRPGDTP | |
| | DAVLFQIATG IAQEITEDVS SWTDLAKNSD RGLKAAHRYF ARVGGFPAFD | |
| | NLTPPATVQP TDTTIDYDPN APFHLVSHAD QTLIHQSISL CAHRIRQEDP | |
| | ALDPNKSGFI KQLQNNELSQ TFYGLSWLFG AGYVHFRECT ANDLAIQYGI | |
| | PNNCRDGIHQ IKSFADAILP NTFFEKKHYR KDSRSVGKKA KSWISNYWQR | |
| | LLQLQTWVDD HTWVTLPQEL TEAQFKPLER GLLVDAVELM AIAERLPQRL | |
| | ADCRDSLDCL MGKGPQAATK NDVEIVEKVR EEIESFVGQI EQLGNQLRHQ | |
| | LENENNDQVH RDNLHQLKNR LPLDLRRPQA LNKISGGVPD VAKSIRGLET | |
| | QLDQVLKERR SHFGRLTKWA KECGITLDPL QPLIESEKQR VAERGSAHDA | |
| | KELAIRLLLQ RIGRLGHRLS PTNATAIQEL LRPVFAVKRE FNLFFHNHMG | |
| | ALYRSPYSTS RHQPFQINVD VAHGTDWIGT IETLIQNLFT QIQDDALLRD | |
| | LVQLEGFVFS HKLRALPGVI PSELARPNNL QQMGLPALLL VLLQADQVHR | |
| | ETVLRVFNLY GSAINGYLFQ ALRPGFIVRA GFQRLETKKL RYVPKAQSWQ | |
| | YPDRLHHAKS AIKNSLSAGW IKKNHQGAIL PQKTLTALVK QKSLKDTGVP | |
| | EYLVQAPHDW YVPIDLRGPA IPIEGLTVGT EGPELTQLGP MKDDCAFRAI | |
| | GPSSFKSKID AGLLPQDVKY GDMTLIFDQH YQQSISFANG TFSIQYQPTS | |
| | LQVKAAIPVV DKRPRDTRNN SHLYDRIVAI DLGERKIGYA IFDLKQVLKS | |
| | EQLEPMREDG KPLIGSISIR SIRGLMKAVQ THRNRRQPNY RIDQTYSKAL | |
| | MHYRESVIGD VCNAIDTLCA RYGGFPVLES SVRNFEVGSA QLKTVYGSVS | |
| | RRYTWSAVDA HKNQRQQYWL GGTKDKIPIW THPYLMTREW DEKNSKWSNR | |
| | SKPLKMHPGV EVHPAGTSQI CHQCKRNPIG ALWNVADTVV LDDQGQLDLD | |
| | DGTIRLNSGY IDTTEIKRAR RKKIRLPENK PLTGSHKTSH VRAVARRNLR | |
| | QPPKSTRAKD TTQSRYTCLY VDCGHECHAD ENAAINIGRK YLQERIHIEA | |
| | SRQALSTR | |
| QFN42174.1 | MVAGLKKIKR DGVTMKSNYH GGVKARAWRK RIGGLARRQK ETVFTYKFPL | (SEQ ID |
| | ETEEAGIDED KAVQTYGIAE GISQGSLIGL VCAFHLSGER LFSKADETKA | NO: 210) |
| | FCNQGRYPNQ AFAEKLRNEL SVTLPKLSPQ SLDVLFQSSP KSKNGVAPEW | |
| | SKNAIRNRLY TNWTGKGAGT NPDEHLLEIA EDIAAEIDSD LDGWKDLEEH | |
| | PEKGLSAADR YFQAQGDFPS LTGLPPSVPL TPQNSTVAFE GDPVCLNPSD | |
| | NTLLHQAVAR CAGRILQEQP NLSPDKNRFI NQLQDELVSS QNNGLSWLFG | |
| | VGFKYWKEMS VDQLADDYKV KSTDLDALKQ VKSFIDAIPL NPLFDTPHYG | |
| | EFRASVAGKM RSWVKNYWKR LLDLKSQLGT ANINLPEGLD EQRAENLESG | |
| | LLIDSKGLRQ VTDKLPSRLK KAEDTIDRLM GDGNPTSDDI EQVETVAAEI | |
| | SAFIGQVEQF NNQLEQRLEN PLEGDDETFL KQLKIDLPAE FKKPPAINRI | |
| | SGGSPDPTAE IAELEEKLDR LMSARKEHYE TIAEWASANK VTLDPMEAMT | |
| | TLEAQRLTER GAEGDQEEFA LRLLLQRIGR LANRLSPQGA TAIRDLLRPV | |
| | FTEKREFNLF FHNRMGSLYR SPYSTSRHQP FTIDVAVAKN TDWMDALDGI | |
| | AETIMKGLSQ AGDELSLRQL EEDEVSREVC LKAFNLYVSA INGCLFRALR | |
| | EGFIVRTKFQ RLERDVLSYV PKTKLWNYPQ RLDTARGPIH SALAAAWINK | |
| | EGSVIDPVET VTALSDTGFS DDGIPEYLVQ APHDWYLRDW INISGFSLSQ | |
| | RLRGLPDTVP GELALVRSAD DVRIPPMLAL TPIDLRDISK PVSGLPVKKN | |
| | ITGLKRQKKQ TAFRMVGPSS FKSHLDSTLL SEEVKLGDFT LIFDQYYKQR | |
| | VSYNGRVKIT FEPDRLHVEA AVPVIDKRVR PSTEEDALFD HLLAIDLGEK | |
| | RVGYAVYDIK ACLRTGDIKP LEDGDGKPIV GSVAVPSIRR LMKAVRSHRQ | |
| | QRQPNQKVNQ TYSTALMNYR ENVIGDVCNR IDTLMEKYNA FPVLESSVMN | |
| | FEAGSRQLEM VYGSVLHRYT YSKIDAHTAK RKEYWYTGEY WDHPYLMAHK | |
| | WNERTRSYSG SLSALTLYPG VMVHPAGTSQ RCHQCKRNPM VEIKQLTGQV | |
| | EINADGSLEL DDGTICLYEG YDYSPEEYKK AKREKRRLDP NVPLSGRHQA | |
| | KHVSAVAKRN LRRPTVSMMS GDTTQARYVC LYTDCDFTGH ADENAAINIG | |
| | WKYLTERIAL SESKDKAGV | |

TABLE 8
Cas12e (CasY) orthologs

| | | |
|---|---|---|
| APG80656.1 | MSKRHPRISG VKGYRLHAQR LEYTGKSGAM RTIKYPLYSS PSGGRTVPRE | (SEQ ID |
| GI: | IVSAINDDYV GLYGLSNFDD LYNAEKRNEE KVYSVLDFWY DCVQYGAVFS | NO: 211) |
| 1110962136 | YTAPGLLKNV AEVRGGSYEL TKTLKGSHLY DELQIDKVIK FLNKKEISRA | |
| QFN42175.1 | NGSLDKLKKD IIDCFKAEYR ERHKDQCNKL ADDIKNAKKD AGASLGERQK | |
| | KLFRDFFGIS EQSENDKPSF TNPLNLTCCL LPFDTVNNNR NRGEVLFNKL | |
| | KEYAQKLDKN EGSLEMWEYI GIGNSGTAFS NFLGEGFLGR LRENKITELK | |
| | KAMMDITDAW RGQEQEEELE KRLRILAALT IKLREPKFDN HWGGYRSDIN | |
| | GKLSSWLQNY INQTVKIKED LKGHKKDLKK AKEMINRFGE SDTKEEAVVS | |
| | SLLESIEKIV PDDSADDEKP DIPAIAIYRR FLSDGRLTLN RFVQREDVQE | |
| | ALIKERLEAE KKKKPKKRKK KSDAEDEKET IDFKELFPHL AKPLKLVPNF | |
| | YGDSKRELYK KYKNAAIYTD ALWKAVEKIY KSAFSSSLKN SFFDTDFDKD | |
| | FFIKRLQKIF SVYRRFNTDK WKPIVKNSFA PYCDIVSLAE NEVLYKPKQS | |
| | RSRKSAAIDK NRVRLPSTEN IAKAGIALAR ELSVAGFDWK DLLKKEEHEE | |
| | YIDLIELHKT ALALLLAVTE TQLDISALDF VENGTVKDFM KTRDGNLVLE | |
| | GRFLEMFSQS IVFSELRGLA GLMSRKEFIT RSAIQTMNGK QAELLYIPHE | |
| | FQSAKITTPK EMSRAFLDLA PAEFATSLEP ESLSEKSLLK LKQMRYYPHY | |
| | FGYELTRTGQ GIDGGVAENA LRLEKSPVKK REIKCKQYKT LGRGQNKIVL | |
| | YVRSSYYQTQ FLEWFLHRPK NVQTDVAVSG SFLIDEKKVK TRWNYDALTV | |
| | ALEPVSGSER VFVSQPFTIF PEKSAEEEGQ RYLGIDIGEY GIAYTALEIT | |
| | GDSAKILDQN FISDPQLKTL REEVKGLKLD QRRGTFAMPS TKIARIRESL | |
| | VHSLRNRIHH LALKHKAKIV YELEVSRFEE GKQKIKKVYA TLKKADVYSE | |
| | IDADKNLQTT VWGKLAVASE ISASYTSQFC GACKKLWRAE MQVDETITTQ | |
| | ELIGTVRVIK GGTLIDAIKD FMRPPIFDEN DTPFPKYRDF CDKHHISKKM | |
| | RGNSCLFICP FCRANADADI QASQTIALLR YVKEEKKVED YFERFRKLKN | |
| | IKVLGQMKKI | |

### 6.4. Protospacer Adjacent Motif

As used herein, the term “protospacer adjacent sequence” or “protospacer adjacent motif” or “PAM” refers to an approximately 2-6 base pair DNA sequence (or a 2-, 3-, 4-, 5-, 6-, 7-, 8-, 9-, 10-, 11-, 12-long nucleotide sequence) that is an important targeting component of a Cas9 nuclease. Typically, the PAM sequence is on either strand, and is downstream in the 5′ to 3′ direction of Cas9 cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease may be modified to alter the PAM specificity of the nuclease such that the nuclease recognizes alternative PAM sequence.

For example, with reference to the canonical SpCas9 amino acid sequence, the PAM specificity can be modified by introducing one or more mutations, including (a) D1135V, R1335Q, and T1337R “the VQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein.

It will also be appreciated that Cas9 enzymes from different bacterial species (i.e., Cas9 orthologs) can have varying PAM specificities and some embodiments are therefore chosen based on the desired PAM recognition. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These examples are not meant to be limiting. It will be further appreciated that non-SpCas9s bind a variety of PAM sequences, which makes them useful to expand the range of sequences that can be targeted according to the invention. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference may be made to Shah et al., “Protospacer recognition motifs: mixed identities and functional diversity,” RNA Biology, 10(5): 891-899 (which is incorporated herein by reference). Gasiunas used cell-free biochemical screens to identify protospacer adjacent motif (PAM) and guide RNA requirements of 79 Cas9 proteins. (Gasiunas et al., A catalogue of biochemically diverse CRISPR-Cas9 orthologs, Nature Communications 11:5512 doi.org/10.1038/s41467-020-19344-1) The authors described 7 classes of gRNA and 50 different PAM requirement.

Oh, Y. et al. describe linking reverse transcriptase to a Francisella novicida Cas9 [FnCas9(H969A)] nickase module. (Oh, Y. et al., Expansion of the prime editing modality with Cas9 from Francisella novicida, bioRxiv 2021.05.25.445577; doi.org/10.1101/2021.05.25.445577). By increasing the distance to the PAM, the FnCas9(H969A) nickase module expands the region of a reverse transcription template (RTT) following the primer binding site.

### 6.5. Prime Editors

“Prime editor fusion protein” describes a protein that is used in prime editing. Prime editing uses CRISPR enzyme that nicks or cuts only single strand of double stranded DNA, i.e., a nickase; and a nickase can occur either naturally or by mutation or modification of a nuclease that makes double stranded cuts. Such an enzyme can be a catalytically-impaired Cas9 endonuclease (a nickase). Such an enzyme can be a Cas12a/b, MAD7, or variant thereof. The nickase is fused to an engineered reverse transcriptase (RT). The nickase is programmed (directed) with a prime-editing guide RNA (pegRNA). The skilled person in the art would appreciate that the pegRNA both specifies the target site and encodes the desired edit. Advantageously the nickase is a catalytically-impaired Cas9 endonuclease, a Cas9 nickase, that is fused to the reverse transcriptase. During genetic editing, the Cas9 nickase part of the protein is guided to the DNA target site by the pegRNA, whereby a nick or single stranded cut occurs. The reverse transcriptase domain then uses the pegRNA to template reverse transcription of the desired edit, directly polymerizing DNA onto the nicked target DNA strand. The edited DNA strand replaces the original DNA strand, creating a heteroduplex containing one edited strand and one unedited strand. Afterward, optionally, the prime editor (PE) guides resolution of the heteroduplex to favor copying the edit onto the unedited strand, completing the process (typically achieved with a nickase gRNA).

As used herein, “PE1” refers to a PE complex comprising a fusion protein comprising Cas9(H840A) and a wild type MMLV RT having the following N-terminus to C-terminus structure: [NLS]-[Cas9(H840A)]-[linker]-[MMLV_RT(wt)]+a desired atgRNA (or PEgRNA). In various embodiments, the prime editors disclosed herein is comprised of PE1.

As used herein, “PE2” refers to a PE complex comprising a fusion protein comprising Cas9(H840A) and a variant MMLV RT having the following N-terminus to C-terminus structure:

[NLS]-[Cas9(H840A)]-[linker]-[MMLV_RT(D200N)(T330P)(L603W)(T306K)(W313F)]+a desired atgRNA (or PEgRNA). In various embodiments, the prime editors disclosed herein are comprised of PE2. In various embodiments, the prime editors disclosed herein is comprised of PE2 and co-expression of MMR protein MLH1dn, that is PE4.

As used herein, “PE3” refers to PE2 plus a second-strand nicking guide RNA that complexes with the PE2 and introduces a nick in the non-edited DNA strand. The induction of the second nick increases the chances of the unedited strand, rather than the edited strand, to be repaired. In various embodiments, the prime editors disclosed herein are comprised of PE3. In various embodiments, the prime editors disclosed herein are comprised of PE3 and co-expression of MMR protein MLH1dn, that is PE5.

As used herein, “PE3b” refers to PE3 but wherein the second-strand nicking guide RNA is designed for temporal control such that the second strand nick is not introduced until after the installation of the desired edit. This is achieved by designing a gRNA with a spacer sequence with mismatches to the unedited original allele that matches only the edited strand. Using this strategy, mismatches between the protospacer and the unedited allele should disfavor nicking by the sgRNA until after the editing event on the PAM strand takes place.

### 6.6. Guides for Prime Editing

Anzalone et al., 2019 (Nature 576:149) describes prime editing and a prime editing complex using a type II CRISPR and can be used herein. A prime editing complex consists of a type II CRISPR PE protein containing an RNA-guided DNA-nicking domain fused to a reverse transcriptase (RT) domain and complexed with a pegRNA. The pegRNA comprises (5′ to 3′) a spacer that is complementary to the target sequence of a genomic DNA, a nickase (e.g. Cas9) binding site, a reverse transcriptase template including editing positions, and primer binding site (PBS). The PE-pegRNA complex binds the target DNA and the CRISPR protein nicks the PAM-containing strand. The resulting 3′ end of the nicked target hybridizes to the primer-binding site (PBS) of the pegRNA, then primes reverse transcription of new DNA containing the desired edit using the RT template of the pegRNA. The overall structure of the pegRNA is like that of a typical type II sgRNA with a reverse transcriptase template/primer binding site appended to the 3′ end. The structure leaves the PBS at the 3′ end of the pegRNA free to bind to the nicked strand complementary to the target which forms the primer for reverse transcription.

Guide RNAs of CRISPRs differ in overall structure. For example, while the spacer of a type II gRNA is located at the 5′ end, the spacer of a type V gRNA is located towards the 3′ end, with the CRISPR protein (e.g. Cas12a) binding region located toward the 5′ end. Accordingly, the regions of a type V pegRNA are rearranged compared to a type II pegRNA. The overall structure of the pegRNA is like that of a typical type II sgRNA with a reverse transcriptase template/primer binding site appended to the 3′ end. The pegRNA comprises (5′ to 3′) a CRISPR protein-binding region, a spacer which is complementary to the target sequence of a genomic DNA, a reverse transcriptase template including editing positions, and primer binding site (PBS).

In typical embodiments, an atgRNA comprises a reverse transcriptase template that encodes, partially or in its entirety, an integration recognition site (also referred to as an integration target recognition site) or a recombinase recognition site (also referred to as a recombinase target recognition site). The integration target recognition site, which is to be placed at a desired location in the genome or intracellular nucleic acid, is referred to as a “beacon” site or an “attachment site” or a “landing pad” or “landing site.” An integration target recognition site or recombinase target recognition site incorporated into the pegRNA is referred to as an attachment site containing guide RNA (atgRNA).

### 6.7. Attachment Site-Containing Guide RNA (atgRNA)

As used herein, the term “attachment site-containing guide RNA” (atgRNA) and the like refer to an extended single guide RNA (sgRNA) comprising a primer binding site (PBS), a reverse transcriptase (RT) template sequence, and wherein the RT template encodes for an integration recognition site or a recombinase recognition site that can be recognized by a recombinase, integrase, or transposase. In some embodiments, the RT template comprises a clamp sequence and an integration recognition site. As referred to herein an atgRNA may be referred to as a guide RNA. An integration recognition site or recombinase target recognition site incorporated into the pegRNA is referred to as an attachment site containing guide RNA (atgRNA).

As used herein, the term “cognate integration recognition site” or “integration cognate” or “cognate pair” refers to a first integration recognition site (e.g., any of the integration recognition sites described herein) and a second integration recognition site (e.g., any of the integration recognition sites described herein) that can be recombined. Recombination between a first integration recognition site (e.g., any of the integration recognition sites described herein) and a second recognition site (e.g., any of the integration recognition sites described herein) is mediated by functional symmetry between the two integration recognition sites and the central dinucleotide of each of the two integration recognition sites. In some cases, a first integration recognition site (e.g., any of the integration recognition sites described herein) that can be recombined with a second integration recognition site (e.g., any of the integration recognition sites described herein) are referred to as a “cognate pair.” A non-limiting example of a cognate pair include an attB site and an attP site, whereby a serine integrase mediates recombination between the attB site and the attP site.

In typical embodiments, an atgRNA comprises a reverse transcriptase template that encodes, partially or in its entirety, an integration recognition site (also referred to as an integration target recognition site) or a recombinase recognition site (also referred to as a recombinase target recognition site). The integration target recognition site, which is to be placed at a desired location in the genome or intracellular nucleic acid, is referred to as a “beacon,” a “beacon” site or an “attachment site” or a “landing pad” or “landing site.” An integration target recognition site or recombinase target recognition site incorporated into the pegRNA is referred to as an attachment site containing guide RNA (atgRNA).

During genome editing, the primer binding site allows the 3′ end of the nicked DNA strand to hybridize to the atgRNA, while the RT template serves as a template for the synthesis of edited genetic information. The atgRNA is capable for instance, without limitation, of (i) identifying the target nucleotide sequence to be edited and (ii) encoding new genetic information that replaces (or in some cases adds) the targeted sequence. In some embodiments, the atgRNA is capable of (i) identifying the target nucleotide sequence to be edited and (ii) encoding an integration site that replaces (or inserts/deletes within) the targeted sequences.

In some embodiments, the co-delivery system described herein includes a polynucleotide sequence encoding an attachment site-containing guide RNA (atgRNA) packaged in an LNP. In some embodiments, the co-delivery system described herein includes a vector comprising a polynucleotide sequence encoding an atgRNA. In some embodiments, the atgRNA comprises a domain that is capable of guiding the prime editor fusion protein to a target sequence, thereby identifying the target nucleotide sequence to be edited; and a reverse transcriptase (RT) template that comprises a first integration recognition site. In some embodiments, the atgRNA comprises a domain that is capable of guiding the prime editor fusion protein (or prime editor system) to a target sequence, thereby identifying the target nucleotide sequence to be edited; and a reverse transcriptase (RT) template that comprises at least a portion first integration recognition site.

In some embodiments, the co-delivery system described herein includes a polynucleotide sequence encoding a first attachment site-containing guide RNA (atgRNA) and a polynucleotide nucleotide sequence encoding a second attachment site-containing guide RNA (atgRNA) packaged into the same LNP. In some embodiments, the co-delivery system described herein includes a polynucleotide sequence encoding a first attachment site-containing guide RNA (atgRNA) packaged into a first LNP and a polynucleotide nucleotide sequence encoding a second attachment site-containing guide RNA (atgRNA) packaged into a second LNP.

In some embodiments, the co-delivery system described herein includes a vector comprising a polynucleotide sequence encoding a first attachment site-containing guide RNA (atgRNA), a polynucleotide sequence encoding a second atgRNA, or both.

In some embodiments, the co-delivery system described herein includes a polynucleotide sequence encoding a first attachment site-containing guide RNA (atgRNA) packaged into a first LNP and a vector comprising a polynucleotide sequence encoding a second atgRNA.

In some embodiments, where the co-delivery system contains a first atgRNA and a second atgRNA, the first atgRNA and the second atgRNA are an at least first pair of atgRNAs, where the at least first pair of atgRNAs have domains that are capable of guiding the gene editor protein or prime editor fusion protein to a target sequence, the first atgRNA further includes a first RT template that comprises at least a portion of the first integration recognition site; and the second atgRNA further includes a second RT template that comprises at least a portion of the first integration recognition site, and the first atgRNA and the second atgRNAs collectively encode the entirety of the first integration recognition site.

In some embodiments, the first atgRNA's reverse transcriptase template encodes for a first single-stranded DNA sequence (i.e., a first DNA flap) that contains a complementary region to a second single-stranded DNA sequence (i.e., a second DNA flap) encoded by a second atgRNA comprising a second reverse transcriptase template. In certain embodiments, the complementary region between the first and second single-stranded DNA sequences is comprised of more than 5 consecutive bases of an integrase target recognition site. In certain embodiments, the complementary region between the first and second single-stranded DNA sequences is comprised of more than 10 consecutive bases of an integrase target recognition site. In certain embodiments, the complementary region between the first and second single-stranded DNA sequences is comprised of more than 20 consecutive bases of an integrase target recognition site. In certain embodiments, the complementary region between the first and second single-stranded DNA sequences is comprised of more than 30 consecutive bases of an integrase target recognition site. Use of two guide RNAs that are (or encode DNA that is) partially complementarity to each other and comprised of consecutive bases of an integrase target recognition site are referred to as dual, paired, annealing, complementary, or twin attachment site-containing guide RNAs (atgRNAs). In certain embodiments, use of two guide RNAs that are (or encode DNA that is) full complementarity to each other and comprised of consecutive bases of an integrase target recognition site are referred to as dual, paired, annealing, complementary, or twin attachment site-containing guide RNAs (atgRNAs).

In some embodiments, upon introducing the nucleic acid construct into a cell, the first atgRNA incorporates the first integration recognition site into the cell's genome at the target sequence.

Table 9 includes atgRNAs, sgRNAs and nicking guides that can be used herein. Spacers are labeled in capital font (SPACER), RT regions in bold capital (RT REGION), AttB sites in bold lower case (attB site), and PBS in capital italics (PBS). Unless otherwise denoted, the AttB is for Bxb1.

TABLE 9

| | | SEQ ID |
|---|---|---|
| Description | Sequence (5′-3′) | NO: |
| ACTB N-term | GCTATTCTCGCAGCTCACCAgttttagagctagaaatagcaagttaaaataaggct | 212 |
| PBS 13 RT | agtccgttatcaacttgaaaaagtggcaccgagtcggtgcGACGAGCGCGGCGAT | |
| 29_AttB 46 | ATCATCATCCATGGccggatgatcctgacgacggagaccgccgtcgtcgacaa | |
| atgRNA | gccggccTGAGCTGCGAGAA | |
| ACTB N-term | GCTATTCTCGCAGCTCACCAgtttgagagctatgctggaaacagcatagcaagtt | 213 |
| PBS 13 RT | caaataaggctagtccgttatcaacttgaaaaagtggcaccgagtcggtgcGACGAGCGC | |
| 29_AttB 46 | GGCGATATCATCATCCATGGccggatgatcctgacgacggagaccgccgt | |
| atgRNA with | cgtcgacaagccggccTGAGCTGCGA GAA | |
| v2 scaffold | | |
| ACTB N-term | GCTATTCTCGCAGCTCACCAgttttagagctagaaatagcaagttaaaataaggct | 214 |
| PBS_13_RT_29_ | agtccgttatcaacttgaaaaagtggcaccgagtcggtgcGAGTCGGTGCGACG | |
| with TP901-1 | AGCGCGGCGATATCATCATCCATGGcacaattaacatctcaatcaag | |
| minimal_AttB f | gtaaaTGCTTGAGCTGCGAGAA | |
| atgRNA | | |
| ACTB N-term | GCTATTCTCGCAGCTCACCAgttttagagctagaaatagcaagttaaaataaggct | \|215 |
| PBS_13_RT_29_ | agtccgttatcaacttgaaaaagtggcaccgagtcggtgcGAGTCGGTGCGACG | |
| with TP901-1 | AGCGCGGCGATATCATCATCCATGGagcatttaccttgattgagatgt | |
| minimal_AttB rc | taattgtgTGAGCTGCGAGAA | |
| atgRNA | | |
| ACTB N-term | GCTATTCTCGCAGCTCACCAgttttagagctagaaatagcaagttaaaataaggct | 216 |
| PBS_13_RT_29_ | agtccgttatcaacttgaaaaagtggcaccgagtcggtgcGAGTCGGTGCGACG | |
| with PhiBT1 | AGCGCGGCGATATCATCATCCATGGcaggtttttgacgaaagtgatc | |
| minimal_AttB f | cagatgatccagTGAGCTGCGAGAA | |
| atgRNA | | |
| ACTB N-term | GCTATTCTCGCAGCTCACCAgttttagagctagaaatagcaagttaaaataaggct | 217 |
| PBS_13_RT_29_ | agtccgttatcaacttgaaaaagtggcaccgagtcggtgcGAGTCGGTGCGACG | |
| with PhiBT1 | AGCGCGGCGATATCATCATCCATGGctggatcatctggatcactttcg | |
| minimal_AttB rc | tcaaaaacctgTGAGCTGCGAGAA | |
| atgRNA | | |
| ACTB N-term | GAAGCCGGCCTTGCACATGCgttttagagctagaaatagcaagttaaaataaggc | 218 |
| Nicking guide 1 | tagtccgttat caacttgaaaaagtggcaccgagtcggtgc | |
| +48 guide | | |
| ACTB N-term | GAAGCCGGCCTTGCACATGCgttttagagctagaaatagcaagttaaaataaggc | 219 |
| PBS_18_RT_16_ | tagtccgttatcaacttgaaaaagtggcaccgagtcggtgcATATCATCATCCATG | |
| with_Lo | GtaccgttcgtatagcatacattatacgaagttatTGAGCTGCGAGAATAGCC | |
| x71_Cre | | |
| atgRNA | | |
| ACTB N-term | GAAGCCGGCCTTGCACATGCgttttagagctagaaatagcaagttaaaataaggc | 220 |
| PBS_13_RT_29_ | tagtccgttatcaacttgaaaaagtggcaccgagtcggtgcGACGAGCGCGGCGA | |
| with_Lo | TATCATCATCCATGGtaccgttcgtatagcatacattatacgaagttatTGAG | |
| x71_Cre | CTGCGAGAA | |
| atgRNA | | |
| ACTB N-term | GCTATTCTCGCAGCTCACCAgttttagagctagaaatagcaagttaaaataaggct | 221 |
| PBS 13 RT | agtccgttatcaacttgaaaaagtggcaccgagtcggtgcTCGACGACGAGCGCG | |
| 34 atgRNA | GCGATATCATCATCCATGGccggatgatcctgacgacggagaccgccgtc | |
| | gtcgacaagccggccTGAGCTGCGAGAA | |
| ACTB N-term | GCTATTCTCGCAGCTCACCAgttttagagctagaaatagcaagttaaaataaggct | 222 |
| PBS 13 RT | agtccgttatcaacttgaaaaagtggcaccgagtcggtgcGAGCGCGGCGATATC | |
| 26 atgRNA | ATCATCCATGGccggatgatcctgacgacggagaccgccgtcgtcgacaagccgg | |
| | ccTGAGCTGCGAGAA | |
| ACTB N-term | GCTATTCTCGCAGCTCACCAgttttagagctagaaatagcaagttaaaataaggct | 223 |
| PBS 13 RT | agtccgttatcaacttgaaaaagtggcaccgagtcggtgcCGCGGCGATATCATC | |
| 23 atgRNA | ATCCATGGccggatgatcctgacgacggagaccgccgtcgtcgacaagccggccT | |
| | GAGCTGCGAGAA | |
| ACTB N-term | GCTATTCTCGCAGCTCACCAgttttagagctagaaatagcaagttaaaataaggct | 224 |
| PBS 13 RT | agtccgttatcaacttgaaaaagtggcaccgagtcggtgcGGCGATATCATCATC | |
| 20 atgRNA | CATGGccggatgatcctgacgacggagaccgccgtcgtcgacaagccggccTGAGC | |
| | TGCGAGAA | |
| ACTB N-term | GCTATTCTCGCAGCTCACCAgttttagagctagaaatagcaagttaaaataaggct | 225 |
| PBS 13 RT | agtccgttatcaacttgaaaaagtggcaccgagtcggtgcATATCATCATCCATG | |
| 16 atgRNA | GccggatgatcctgacgacggagaccgccgtcgtcgacaagccggccTGAGCTGCG | |
| | AGAA | |
| ACTB N-term | GCTATTCTCGCAGCTCACCAgttttagagctagaaatagcaagttaaaataaggct | 226 |
| PBS 18 RT | agtccgttatcaacttgaaaaagtggcaccgagtcggtgcTCGACGACGAGCGCG | |
| 34 atgRNA | GCGATATCATCATCCATGGccggatgatcctgacgacggagaccgccgtc | |
| | gtcgacaagccggccTGAGCTGCGAGAATAGCC | |
| ACTB N-term | GCTATTCTCGCAGCTCACCAgttttagagctagaaatagcaagttaaaataaggct | 227 |
| PBS 18 RT | agtccgttatcaacttgaaaaagtggcaccgagtcggtgcGACGAGCGCGGCGAT | |
| 29 atgRNA | ATCATCATCCATGGccggatgatcctgacgacggagaccgccgtcgtcgacaa | |
| | gccggccTGAGCTGCGAGAATAGCC | |
| ACTB N-term | GCTATTCTCGCAGCTCACCAgttttagagctagaaatagcaagttaaaataaggct | 228 |
| PBS 18 RT | agtccgttatcaacttgaaaaagtggcaccgagtcggtgcATATCATCATCCATG | |
| 16 atgRNA | GccggatgatcctgacgacggagaccgccgtcgtcgacaagccggccTGAGCTGCG | |
| | AGAATAGCC | |
| LMNB1 N-term | GCTGTCTCCGCCGCCCGCCAgttttagagctagaaatagcaagttaaaataaggc | 229 |
| PBS 13 RT 39 | tagtccgttatcaacttgaaaaagtggcaccgagtcggtgcCTGCCCATCCGCGGC | |
| atgRNA | GGCACGGGGGTCGCAGTCGCCATGccggatgatcctgacgacggag | |
| | accgccgtcgtcgacaagccggccCGGGCGGCGGAGA | |
| LMNB1 N-term | GCTGTCTCCGCCGCCCGCCAgttttagagctagaaatagcaagttaaaataaggc | 230 |
| PBS 13 RT 34 | tagtccgttatcaacttgaaaaagtggcaccgagtcggtgcCATCCGCGGCGGCAC | |
| atgRNA | GGGGGTCGCAGTCGCCATGccggatgatcctgacgacggagaccgccgt | |
| | cgtcgacaagccggccCGGGCGGCGGAGA | |
| LMNB1 N-term | GCTGTCTCCGCCGCCCGCCAgttttagagctagaaatagcaagttaaaataaggc | 231 |
| PBS 13 RT 29 | tagtccgttatcaacttgaaaaagtggcaccgagtcggtgcGCGGCGGCACGGGG | |
| atgRNA | GTCGCAGTCGCCATGccggatgatcctgacgacggagaccgccgtcgtcgac | |
| | aagccggccCGGGCGGCGGAGA | |
| LMNB1 N-term | GCTGTCTCCGCCGCCCGCCAgttttagagctagaaatagcaagttaaaataaggc | 232 |
| PBS 13 RT 24 | tagtccgttatcaacttgaaaaagtggcaccgagtcggtgcGGCACGGGGGTCGC | |
| atgRNA | AGTCGCCATGccggatgatcctgacgacggagaccgccgtcgtcgacaagccggc | |
| | cCGGGCGGCGGAGA | |
| LMNB1 N-term | GCTGTCTCCGCCGCCCGCCAgttttagagctagaaatagcaagttaaaataaggc | 233 |
| PBS 13 RT 19 | tagtccgttatcaacttgaaaaagtggcaccgagtcggtgcGGGGGTCGCAGTCG | |
| atgRNA | CCATGccggatgatcctgacgacggagaccgccgtcgtcgacaagccggccCGGG | |
| | CGGCGGAGA | |
| LMNB1 N-term | GCTGTCTCCGCCGCCCGCCAgttttagagctagaaatagcaagttaaaataaggc | 234 |
| PBS 18 RT 39 | tagtccgttatcaacttgaaaaagtggcaccgagtcggtgcCTGCCCATCCGCGGC | |
| atgRNA | GGCACGGGGGTCGCAGTCGCCATGccggatgatcctgacgacggag | |
| | accgccgtcgtcgacaagccggccCGGGCGGCGGAGACAGCG | |
| LMNB1 N-term | GCTGTCTCCGCCGCCCGCCAgttttagagctagaaatagcaagttaaaataaggc | 235 |
| PBS 18 RT 34 | tagtccgttatcaacttgaaaaagtggcaccgagtcggtgcCATCCGCGGCGGCAC | |
| atgRNA | GGGGGTCGCAGTCGCCATGccggatgatcctgacgacggagaccgccgt | |
| | cgtcgacaagccggccCGGGCGGCGGAGACAGCG | |
| LMNB1 N-term | GCTGTCTCCGCCGCCCGCCAgttttagagctagaaatagcaagttaaaataaggc | 236 |
| PBS 18 RT 29 | tagtccgttatcaacttgaaaaagtggcaccgagtcggtgcGCGGCGGCACGGGG | |
| atgRNA | GTCGCAGTCGCCATGccggatgatcctgacgacggagaccgccgtcgtcgac | |
| | aagccggccCGGGCGGCGGAGACAGCG | |
| LMNB1 N-term | GCTGTCTCCGCCGCCCGCCAgttttagagctagaaatagcaagttaaaataaggc | 237 |
| PBS 18 RT 24 | tagtccgttatcaacttgaaaaagtggcaccgagtcggtgcGGCACGGGGGTCGC | |
| atgRNA | AGTCGCCATGccggatgatcctgacgacggagaccgccgtcgtcgacaagccggc | |
| | cCGGGCGGCGGAGACAGCG | |
| LMNB1 N-term | GCTGTCTCCGCCGCCCGCCAgttttagagctagaaatagcaagttaaaataaggc | 238 |
| PBS 18 RT 19 | tagtccgttatcaacttgaaaaagtggcaccgagtcggtgcGGGGGTCGCAGTCG | |
| atgRNA | CCATGccggatgatcctgacgacggagaccgccgtcgtcgacaagccggccCGGG | |
| | CGGCGGAGACAGCG | |
| LMNB1 N-term | GCGTGGTGGGGCCGCCAGCGgttttagagctagaaatagcaagttaaaataagg | 239 |
| Nicking guide 1 | ctagtccgttatcaacttgaaaaagtggcaccgagtcggtgc | |
| +46 | | |
| ACTB N-term | GCTATTCTCGCAGCTCACCAgttttagagctagaaatagcaagttaaaataaggct | 240 |
| PBS 13 RT | agtccgttatcaacttgaaaaagtggcaccgagtcggtgcGACGAGCGCGGCGAT | |
| 29_AttB 42 | ATCATCATCCATGGggatgatcctgacgacggagaccgccgtcgtcgacaagc | |
| atgRNA | cggTGAGCTGCGAGAA | |
| ACTB N-term | GCTATTCTCGCAGCTCACCAgttttagagctagaaatagcaagttaaaataaggct | 241 |
| PBS 13 RT | agtccgttatcaacttgaaaaagtggcaccgagtcggtgcGACGAGCGCGGCGAT | |
| 29_AttB 40 | ATCATCATCCATGGgatgatcctgacgacggagaccgccgtcgtcgacaagcc | |
| atgRNA | gTGAGCTGCGAGAA | |
| ACTB N-term | GCTATTCTCGCAGCTCACCAgttttagagctagaaatagcaagttaaaataaggct | 242 |
| PBS 13 RT | agtccgttatcaacttgaaaaagtggcaccgagtcggtgcGACGAGCGCGGCGAT | |
| 29_AttB 38 | ATCATCATCCATGGatgatcctgacgacggagaccgccgtcgtcgacaagccT | |
| atgRNA | GAGCTGCGAGAA | |
| ACTB N-term | GCTATTCTCGCAGCTCACCAgttttagagctagaaatagcaagttaaaataaggct | 243 |
| PBS 13 RT | agtccgttatcaacttgaaaaagtggcaccgagtcggtgcGACGAGCGCGGCGAT | |
| 29_AttB 36 | ATCATCATCCATGGtgatcctgacgacggagaccgccgtcgtcgacaagcTG | |
| atgRNA | AGCTGCGAGAA | |
| LMNB1 N-term | GCTGTCTCCGCCGCCCGCCAgttttagagctagaaatagcaagttaaaataaggc | 244 |
| PBS 13 | tagtccgttatcaacttgaaaaagtggcaccgagtcggtgcGCGGCGGCACGGGG | |
| RT 29_AttB 44 | GTCGCAGTCGCCATGcggatgatcctgacgacggagaccgccgtcgtcgaca | |
| atgRNA v2 | agccggcCGGGCGGCGGAGA | |
| LMNB1 N-term | GCTGTCTCCGCCGCCCGCCAgttttagagctagaaatagcaagttaaaataaggc | 245 |
| PBS 13 | tagtccgttatcaacttgaaaaagtggcaccgagtcggtgcGCGGCGGCACGGGG | |
| RT 29_AttB 42 | GTCGCAGTCGCCATGggatgatcctgacgacggagaccgccgtcgtcgacaa | |
| atgRNA v2 | gccggCGGGCGGCGGAGA | |
| LMNB1 N-term | GCTGTCTCCGCCGCCCGCCAgttttagagctagaaatagcaagttaaaataaggc | 246 |
| PBS 13 | tagtccgttatcaacttgaaaaagtggcaccgagtcggtgcGCGGCGGCACGGGG | |
| RT 29_AttB 40 | GTCGCAGTCGCCATGgatgatcctgacgacggagaccgccgtcgtcgacaag | |
| atgRNA v2 | ccgCGGGCGGCGGAGA | |
| LMNB1 N-term | GCTGTCTCCGCCGCCCGCCAgttttagagctagaaatagcaagttaaaataaggc | 247 |
| PBS 13 | tagtccgttatcaacttgaaaaagtggcaccgagtcggtgcGCGGCGGCACGGGG | |
| RT 29_AttB 38 | GTCGCAGTCGCCATGatgatcctgacgacggagaccgccgtcgtcgacaagc | |
| atgRNA v2 | cCGGGCGGCGGAGA | |
| NOLC1 N-term | GCGTATTGCCTGGAGGATGGgttttagagctagaaatagcaagttaaaataaggc | 248 |
| PBS 18 | tagtccgttatcaacttgaaaaagtggcaccgagtcggtgcGAACCACGCGGCGA | |
| RT 29_AttB 46 | ATGCCGGCGTCCGCCccggatgatcctgacgacggagaccgccgtcgtcgac | |
| atgRNA | aagccggccTCCTCCAGGCAATACGCG | |
| NOLC1 N-term | GCGTATTGCCTGGAGGATGGgttttagagctagaaatagcaagttaaaataaggc | 249 |
| PBS 13 | tagtccgttatcaacttgaaaaagtggcaccgagtcggtgcGAACCACGCGGCGA | |
| RT 29_AttB 46 | ATGCCGGCGTCCGCCccggatgatcctgacgacggagaccgccgtcgtcgac | |
| atgRNA | aagccggccTCCTCCAGGCAAT | |
| NOLC1 N-term | GCGTATTGCCTGGAGGATGGgttttagagctagaaatagcaagttaaaataaggc | 250 |
| PBS 13 | tagtccgttatcaacttgaaaaagtggcaccgagtcggtgcGAACCACGCGGCGA | |
| RT 29_AttB 44 | ATGCCGGCGTCCGCCcggatgatcctgacgacggagaccgccgtcgtcgaca | |
| atgRNA | agccggcTCCTCCAGGCAAT | |
| NOLC1 N-term | GCGTATTGCCTGGAGGATGGgttttagagctagaaatagcaagttaaaataaggc | 251 |
| PBS 13 | tagtccgttatcaacttgaaaaagtggcaccgagtcggtgcGAACCACGCGGCGA | |
| RT 29_AttB 42 | ATGCCGGCGTCCGCCggatgatcctgacgacggagaccgccgtcgtcgacaa | |
| atgRNA | gccggTCCTCCAGGCAAT | |
| NOLC1 N-term | GCGTATTGCCTGGAGGATGGgttttagagctagaaatagcaagttaaaataaggc | 252 |
| PBS 13 | tagtccgttatcaacttgaaaaagtggcaccgagtcggtgcGAACCACGCGGCGA | |
| RT 29_AttB 40 | ATGCCGGCGTCCGCCgatgatcctgacgacggagaccgccgtcgtcgacaag | |
| atgRNA | ccgTCCTCCAGGCAAT | |
| NOLC1 N-term | GCGTATTGCCTGGAGGATGGgttttagagctagaaatagcaagttaaaataaggc | 253 |
| PBS 13 | tagtccgttatcaacttgaaaaagtggcaccgagtcggtgcGAACCACGCGGCGA | |
| RT 29_AttB 38 | ATGCCGGCGTCCGCCatgatcctgacgacggagaccgccgtcgtcgacaagc | |
| atgRNA | cTCCTCCAGGCAAT | |
| NOLC1 nicking | GAGCCGAGCACGAGGGGATACgttttagagctagaaatagcaagttaaaataa | 254 |
| guide -43 | ggctagtccgttatcaacttgaaaaagtggcaccgagtcggtgc | |
| ACTB N-term | GCTATTCTCGCAGCTCACCAgttttagagctagaaatagcaagttaaaataaggct | 255 |
| PBS 13 RT | agtccgttatcaacttgaaaaagtggcaccgagtcggtgcGGCGATATCATCATC | |
| 20_AttB 38 | CATGGatgatcctgacgacggagaccgccgtcgtcgacaagccTGAGCTGCGA | |
| atgRNA | GAA | |
| ACTB N-term | GCTATTCTCGCAGCTCACCAgttttagagctagaaatagcaagttaaaataaggct | 256 |
| PBS 13 RT | agtccgttatcaacttgaaaaagtggcaccgagtcggtgcTATCATCATCCATGGa | |
| 15_AttB 38 | tgatcctgacgacggagaccgccgtcgtcgacaagccTGAGCTGCGAGAA | |
| atgRNA | | |
| ACTB N-term | GCTATTCTCGCAGCTCACCAgttttagagctagaaatagcaagttaaaataaggct | 257 |
| PBS 13 RT | agtccgttatcaacttgaaaaagtggcaccgagtcggtgcTCATCCATGGatgatcctg | |
| 10_AttB 38 | acgacggagaccgccgtcgtcgacaagccTGAGCTGCGAGAA | |
| atgRNA | | |
| ACTB N-term | GCTATTCTCGCAGCTCACCAgttttagagctagaaatagcaagttaaaataaggct | 258 |
| PBS 9 RT | agtccgttatcaacttgaaaaagtggcaccgagtcggtgcGGCGATATCATCATC | |
| 20_AttB 38 | CATGGatgatcctgacgacggagaccgccgtcgtcgacaagccTGAGCTGCG | |
| atgRNA | | |
| ACTB N-term | GCTATTCTCGCAGCTCACCAgttttagagctagaaatagcaagttaaaataaggct | 259 |
| PBS 9 RT | agtccgttatcaacttgaaaaagtggcaccgagtcggtgcTATCATCATCCATGGa | |
| 15_AttB 38 | tgatcctgacgacggagaccgccgtcgtcgacaagccTGAGCTGCG | |
| atgRNA | | |
| ACTB N-term | GCTATTCTCGCAGCTCACCAgttttagagctagaaatagcaagttaaaataaggct | 260 |
| PBS 9 RT | agtccgttatcaacttgaaaaagtggcaccgagtcggtgcTCATCCATGGatgatcctg | |
| 10_AttB 38 | acgacggagaccgccgtcgtcgacaagccTGAGCTGCG | |
| atgRNA | | |
| LMNB1 N-term | GCTGTCTCCGCCGCCCGCCAgttttagagctagaaatagcaagttaaaataaggc | 261 |
| PBS 13 | tagtccgttatcaacttgaaaaagtggcaccgagtcggtgcCGGGGGTCGCAGTC | |
| RT 20_AttB 38 | GCCATGatgatcctgacgacggagaccgccgtcgtcgacaagccCGGGCGGCG | |
| atgRNA | GAGA | |
| LMNB1 N-term | GCTGTCTCCGCCGCCCGCCAgttttagagctagaaatagcaagttaaaataaggc | 262 |
| PBS 13 | tagtccgttatcaacttgaaaaagtggcaccgagtcggtgcGTCGCAGTCGCCATG | |
| RT 15_AttB 38 | atgatcctgacgacggagaccgccgtcgtcgacaagccCGGGCGGCGGAGA | |
| atgRNA | | |
| LMNB1 N-term | GCTGTCTCCGCCGCCCGCCAgttttagagctagaaatagcaagttaaaataaggc | 263 |
| PBS 13 | tagtccgttatcaacttgaaaaagtggcaccgagtcggtgcAGTCGCCATGatgatcct | |
| RT 10_AttB 38 | gacgacggagaccgccgtcgtcgacaagccCGGGCGGCGGAGA | |
| atgRNA | | |
| LMNB1 N-term | GCTGTCTCCGCCGCCCGCCAgttttagagctagaaatagcaagttaaaataaggc | 264 |
| PBS 9 | tagtccgttatcaacttgaaaaagtggcaccgagtcggtgcCGGGGGTCGCAGTC | |
| RT 20_AttB 38 | GCCATGatgatcctgacgacggagaccgccgtcgtcgacaagccCGGGCGGCG | |
| atgRNA | | |
| LMNB1 N-term | GCTGTCTCCGCCGCCCGCCAgttttagagctagaaatagcaagttaaaataaggc | 265 |
| PBS 9 | tagtccgttatcaacttgaaaaagtggcaccgagtcggtgcGTCGCAGTCGCCATG | |
| RT 15_AttB 38 | atgatcctgacgacggagaccgccgtcgtcgacaagccCGGGCGGCG | |
| atgRNA | | |
| LMNB1 N-term | GCTGTCTCCGCCGCCCGCCAgttttagagctagaaatagcaagttaaaataaggc | 266 |
| PBS 9 | tagtccgttatcaacttgaaaaagtggcaccgagtcggtgcAGTCGCCATGatgatcct | |
| RT 10_AttB 38 | gacgacggagaccgccgtcgtcgacaagccCGGGCGGCG | |
| atgRNA | | |
| SUPT16H N- | GAGAAGCGGCGTCCGGGGCTAgttttagagctagaaatagcaagttaaaataag | 267 |
| term PBS 13 | gctagtccgttatcaacttgaaaaagtggcaccgagtcggtgcTCTTTGTCCAGAGT | |
| RT 24 Bxb1- | CACAGCCATAccggatgatcctgacgacggagaccgccgtcgtcgacaagccggc | |
| GT_Initial | cCCCCGGACGCCGC | |
| length | | |
| SRRM2 N-term | GGGCACGGGGCCATGTACAAgttttagagctagaaatagcaagttaaaataagg | 268 |
| PBS 13 | ctagtccgttatcaacttgaaaaagtggcaccgagtcggtgcGGCGTCGGCAGCCC | |
| RT 24 Bxb1 | GATCCCGTTGccggatgatcctgacgacggagaccgccgtcgtcgacaagccggc | |
| Initial length | cTACATGGCCCCGT | |
| DEPDC4 N- | GTGTCAGGTGGGGCGGGGCTAgttttagagctagaaatagcaagttaaaataag | 269 |
| term PBS 18 | gctagtccgttatcaacttgaaaaagtggcaccgagtcggtgcGCTGGCTCCTCCCC | |
| RT 24 Bxb1 | TGGCACCATAccggatgatcctgacgacggagaccgccgtcgtcgacaagccggc | |
| Initial length | cCCCCGCCCCACCTGACAC | |
| NES N-term | GAGTGGGTCAGACGAGCAGGAgttttagagctagaaatagcaagttaaaataa | 270 |
| PBS 13 RT | ggctagtccgttatcaacttgaaaaagtggcaccgagtcggtgcCGACTCCTCCCCC | |
| 29 Bxb1 Initial | ATGCAGCCCTCCATCccggatgatcctgacgacggagaccgccgtcgtcgaca | |
| length | agccggccTGCTCGTCTGACC | |
| SUPT16H | GCAGCCACCCGCTCTCGGCCCgttttagagctagaaatagcaagttaaaataag | 271 |
| nicking guide - | gctagtccgttatcaacttgaaaaagtggcaccgagtcggtgc | |
| 53 | | |
| SRRM2 N-term | GTGTAGTCAGGCCGCTCACCCgttttagagctagaaatagcaagttaaaataag | 272 |
| nicking guide 1 | gctagtccgttatcaacttgaaaaagtggcaccgagtcggtgc | |
| +87 | | |
| DEPDC4 N- | GCTGACAAGTCTACGGAACCTgttttagagctagaaatagcaagttaaaataag | 273 |
| term Nicking | gctagtccgttatcaacttgaaaaagtggcaccgagtcggtgc | |
| guide 1 +59 | | |
| NES N-term | GCTCCTCCAGCGCCTTGACCgttttagagctagaaatagcaagttaaaataaggct | 274 |
| Nicking guide 2 | agtccgttatcaacttgaaaaagtggcaccgagtcggtgc | |
| +79 | | |
| HITI_ACTB_guide | GCTATTCTCGCAGCTCACCAgttttagagctagaaatagcaagttaaaataaggct | 275 |
| | agtccgttatcaacttgaaaaagtggcaccgagtcggtgc | |
| HITI_SUPTH16_ | AGAAGCGGCGTCCGGGGCTAgttttagagctagaaatagcaagttaaaataagg | 276 |
| guide | ctagtccgttatcaacttgaaaaagtggcaccgagtcggtgc | |
| HITI_SRRM2_ | GGGCACGGGGCCATGTACAAgttttagagctagaaatagcaagttaaaataagg | 277 |
| guide | ctagtccgttatcaacttgaaaaagtggcaccgagtcggtgc | |
| HITI_NOLC1_ | GCGTATTGCCTGGAGGATGGgttttagagctagaaatagcaagttaaaataaggc | 278 |
| guide | tagtccgttatcaacttgaaaaagtggcaccgagtcggtgc | |
| HITI_DEPDC4_ | TGTCAGGTGGGGGGGGCTAgttttagagctagaaatagcaagttaaaataagg | 279 |
| guide | ctagtccgttatcaacttgaaaaagtggcaccgagtcggtgc | |
| HITI_NES_guide | AGTGGGTCAGACGAGCAGGAgttttagagctagaaatagcaagttaaaataagg | 280 |
| | ctagtccgttatcaacttgaaaaagtggcaccgagtcggtgc | |
| HITI_LMNB1_ | GCTGTCTCCGCCGCCCGCCAgttttagagctagaaatagcaagttaaaataaggc | 281 |
| guide | tagtccgttatcaacttgaaaaagtggcaccgagtcggtgc | |
| HDR Cas9 | GCTATTCTCGCAGCTCACCAgttttagagctagaaatagcaagttaaaataaggct | 275 |
| ACTB guide | agtccgttatcaacttgaaaaagtggcaccgagtcggtgc | |
| HDR Cas9 | GGGGTCGCAGTCGCCATGGCgttttagagctagaaatagcaagttaaaataagg | 282 |
| LMNB1 guide | ctagtccgttatcaacttgaaaaagtggcaccgagtcggtgc | |
| ACTB N-term | GCTATTCTCGCAGCTCACCAgttttagagctagaaatagcaagttaaaataaggct | 283 |
| PBS 13 RT | agtccgttatcaacttgaaaaagtggcaccgagtcggtgcGACGAGCGCGGCGAT | |
| 29_AttB original | ATCATCATCCATGGccggatgatcctgacgacggagXXcgccgtcgtcgaca | |
| length atgRNAs | agccggccTGAGCTGCGAGAA | |
| for | XX: CG, GC, AT, TA, GG, TT, GA, AG, CC, TC, CT, AA, TG, GT, CA, AC | |
| dinucleotides | | |
| ACTB N-term | GCTATTCTCGCAGCTCACCAgttttagagctagaaatagcaagttaaaataaggct | 284 |
| PBS 13 RT | agtccgttatcaacttgaaaaagtggcaccgagtcggtgcGACGAGCGCGGCGAT | |
| 29 atgRNA | ATCATCATCCATGccggatgatcctgacgacggagACcgccgtcgtcgacaag | |
| with_AttB 46 | ccggccTGAGCTGCGAGAA | |
| GT for fusion | | |
| ACTB N-term | GCTATTCTCGCAGCTCACCAgttttagagctagaaatagcaagttaaaataaggct | 285 |
| PBS 13 RT | agtccgttatcaacttgaaaaagtggcaccgagtcggtgcGACGAGCGCGGCGAT | |
| 29 atgRNA | ATCATCATCCATGccggatgatcctgacgacggagAGcgccgtcgtcgacaag | |
| with_AttB 46 | ccggccTGAGCTGCGAGAA | |
| CT for | | |
| multiplexing | | |
| NOLC1 N-term | GCGTATTGCCTGGAGGATGGgttttagagctagaaatagcaagttaaaataaggc | 286 |
| PBS 18 | tagtccgttatcaacttgaaaaagtggcaccgagtcggtgcGAACCACGCGGCGA | |
| RT 29 atgRNA | ATGCCGGCGTCCGCCccggatgatcctgacgacggagTCcgccgtcgtcga | |
| with_AttB 46 | caagccggccTCCTCCAGGCAATACGCG | |
| GA for | | |
| multiplexing | | |
| LMNB1 N-term | GCTGTCTCCGCCGCCCGCCAgttttagagctagaaatagcaagttaaaataaggc | 287 |
| PBS 18 | tagtccgttatcaacttgaaaaagtggcaccgagtcggtgcGCGGCGGCACGGGG | |
| RT 29 atgRNA | GTCGCAGTCGCCATGccggatgatcctgacgacggagCTcgccgtcgtcga | |
| with_AttB 46 | caagccggccCGGGCGGCGGAGACAGCG | |
| AG for | | |
| multiplexing | | |
| EMX1 Cas9 | GTCACCTCCAATGACTAGGGgttttagagctagaaatagcaagttaaaataaggc | 288 |
| guide 1 | tagtccgttatcaacttgaaaaagtggcaccgagtcggtgc | |
| EMX1 Cas9 | GGGCAACCACAAACCCACGAgttttagagctagaaatagcaagttaaaataagg | 289 |
| guide 2 | ctagtccgttatcaacttgaaaaagtggcaccgagtcggtgc | |
| ACTB N-term | GCTATTCTCGCAGCTCACCAgttttagagctagaaatagcaagttaaaataaggct | 290 |
| PBS 13 RT | agtccgttatcaacttgaaaaagtggcaccgagtcggtgcGACGAGCGCGGCGAT | |
| 29_AttB 56 GA | ATCATCATCCATGGctatgccggatgatcctgacgacggagtccgccgtcgtcg | |
| atgRNA | acaagccggccctagcTGAGCTGCGAGAA | |
| ACTB N-term | GCTATTCTCGCAGCTCACCAgttttagagctagaaatagcaagttaaaataaggct | 291 |
| PBS 13 RT | agtccgttatcaacttgaaaaagtggcaccgagtcggtgcGACGAGCGCGGCGAT | |
| 29_AttB 51 GA | ATCATCATCCATGGtgccggatgatcctgacgacggagtccgccgtcgtcgaca | |
| atgRNA | agccggccctaTGAGCTGCGAGAA | |
| ACTB N-term | GCTATTCTCGCAGCTCACCAgttttagagctagaaatagcaagttaaaataaggct | 292 |
| PBS 13 RT | agtccgttatcaacttgaaaaagtggcaccgagtcggtgcGACGAGCGCGGCGAT | |
| 29_AttB 46 GA | ATCATCATCCATGGccggatgatcctgacgacggagtccgccgtcgtcgacaa | |
| atgRNA | gccggccTGAGCTGCGAGAA | |
| ACTB N-term | GCTATTCTCGCAGCTCACCAgttttagagctagaaatagcaagttaaaataaggct | \|293 |
| PBS 13 RT | agtccgttatcaacttgaaaaagtggcaccgagtcggtgcGACGAGCGCGGCGATA | |
| 29_AttB 41 GA | TCATCATCCATGGggatgatcctgacgacggagtccgccgtcgtcgacaagccgT | |
| atgRNA | GAGCTGCGAGAA | |
| ACTB N-term | GCTATTCTCGCAGCTCACCAgttttagagctagaaatagcaagttaaaataaggct | 294 |
| PBS 13 RT | agtccgttatcaacttgaaaaagtggcaccgagtcggtgcGACGAGCGCGGCGAT | |
| 29_AttB 36 GA | ATCATCATCCATGGtgatcctgacgacggagtccgccgtcgtcgacaagcTGA | |
| atgRNA | GCTGCGAGAA | |
| ACTB N-term | GCTATTCTCGCAGCTCACCAgttttagagctagaaatagcaagttaaaataaggct | 295 |
| PBS 13 RT | agtccgttatcaacttgaaaaagtggcaccgagtcggtgcGACGAGCGCGGCGAT | |
| 29_AttB 31 GA | ATCATCATCCATGGatcctgacgacggagtccgccgtcgtcgacaTGAGCT | |
| atgRNA | GCGAGAA | |
| ACTB N-term | GCTATTCTCGCAGCTCACCAgttttagagctagaaatagcaagttaaaataaggct | 296 |
| PBS 13 RT | agtccgttatcaacttgaaaaagtggcaccgagtcggtgcGACGAGCGCGGCGAT | |
| 29_AttB 26 GA | ATCATCATCCATGGcctgacgacggagtccgccgtcgtcgTGAGCTGCG | |
| atgRNA | AGAA | |
| ACTB N-term | GCTATTCTCGCAGCTCACCAgttttagagctagaaatagcaagttaaaataaggct | 297 |
| PBS 13 RT | agtccgttatcaacttgaaaaagtggcaccgagtcggtgcGACGAGCGCGGCGATA | |
| 29_AttB 21 GA | TCATCATCCATGGtgacgacggagtccgccgtcgTGAGCTGCGAGAA | |
| atgRNA | | |
| ACTB N-term | GCTATTCTCGCAGCTCACCAgttttagagctagaaatagcaagttaaaataaggct | 298 |
| PBS 13 RT | agtccgttatcaacttgaaaaagtggcaccgagtcggtgcGACGAGCGCGGCGAT | |
| 29_AttB 16 GA | ATCATCATCCATGGacgacggagtccgccgTGAGCTGCGAGAA | |
| atgRNA | | |
| ACTB N-term | GCTATTCTCGCAGCTCACCAgttttagagctagaaatagcaagttaaaataaggct | 299 |
| PBS 13 RT | agtccgttatcaacttgaaaaagtggcaccgagtcggtgcGACGAGCGCGGCGAT | |
| 29_AttB 11 GA | ATCATCATCCATGGgacggagtccgTGAGCTGCGAGAA | |
| atgRNA | | |
| ACTB N-term | GCTATTCTCGCAGCTCACCAgttttagagctagaaatagcaagttaaaataaggct | 300 |
| PBS 13 RT | agtccgttatcaacttgaaaaagtggcaccgagtcggtgcGACGAGCGCGGCGAT | |
| 29_AttB 6 GA | ATCATCATCCATGGcggagtTGAGCTGCGAGAA | |
| atgRNA | | |
| ACTB N-term | GAAGCCGGCCTTGCACATGCgttttagagctagaaatagcaagttaaaataaggc | 301 |
| PBS_18_RT_34_ | tagtccgttatcaacttgaaaaagtggcaccgagtcggtgcTCGACGACGAGCGC | |
| with Lo | GGCGATATCATCATCCATGGtaccgttcgtatagcatacattatacgaagt | |
| x71_Cre | tatTGAGCTGCGAGAATAGCC | |
| atgRNA | | |
| ACTB N-term | GAAGCCGGCCTTGCACATGCgttttagagctagaaatagcaagttaaaataaggc | 302 |
| PBS_18_RT_29_ | tagtccgttatcaacttgaaaaagtggcaccgagtcggtgcGACGAGCGCGGCGA | |
| with Lo | TATCATCATCCATGGtaccgttcgtatagcatacattatacgaagttatTGAG | |
| x71_Cre | CTGCGAGAATAGCC | |
| atgRNA | | |
| ACTB N-term | GAAGCCGGCCTTGCACATGCgttttagagctagaaatagcaagttaaaataaggc | 303 |
| PBS_13_RT_34_ | tagtccgttatcaacttgaaaaagtggcaccgagtcggtgcTCGACGACGAGCGC | |
| with Lo | GGCGATATCATCATCCATGGtaccgttcgtatagcatacattatacgaagt | |
| x71_Cre | tatTGAGCTGCGAGAA | |
| atgRNA | | |
| ACTB N-term | GAAGCCGGCCTTGCACATGCgttttagagctagaaatagcaagttaaaataaggc | 304 |
| PBS_13_RT_16_ | tagtccgttatcaacttgaaaaagtggcaccgagtcggtgcATATCATCATCCATG | |
| with Lo | GtaccgttcgtatagcatacattatacgaagttatTGAGCTGCGAGAA | |
| x71_Cre | | |
| atgRNA | | |
| ACTB N-term | CCCCACGATGGAGGGGAAGAgttttagagctagaaatagcaagttaaaataagg | 305 |
| Nicking guide 2 | ctagtccgttatcaacttgaaaaagtggcaccgagtcggtgc | |
| +93 guide | | |
| LMNB1 N-term | CCTTCTCCTGGAGCCGCGACgttttagagctagaaatagcaagttaaaataaggc | 306 |
| Nicking guide 2 | tagtccgttatcaacttgaaaaagtggcaccgagtcggtgc | |
| +87 guide | | |
| ACTB N-term | GCTATTCTCGCAGCTCACCAgttttagagctagaaatagcaagttaaaataaggct | 307 |
| PBS 13 RT 29 | agtccgttatcaacttgaaaaagtggcaccgagtcggtgcGACGAGCGCGGCGAT | |
| AttB 46 | ATCATCATCCATGGcattatatgttcttacagtatggcggcccggattgtaaaaa | |
| N191352_143_ | catataatgTGAGCTGCGAGAA | |
| 72 integrase | | |
| ACTB N-term | GCTATTCTCGCAGCTCACCAgttttagagctagaaatagcaagttaaaataaggct | 308 |
| PBS 13 RT | agtccgttatcaacttgaaaaagtggcaccgagtcggtgcGACGAGCGCGGCGAT | |
| 29_AttB 46 | ATCATCATCCATGGcgttatagggtattacagtatggcggtcggtactgcaatac | |
| N684346_90_69 | cctataacgTGAGCTGCGAGAA | |
| integrase | | |
| ACTB N-term | GCTATTCTCGCAGCTCACCAgttttagagctagaaatagcaagttaaaataaggct | 309 |
| PBS 13 RT 29 | agtccgttatcaacttgaaaaagtggcaccgagtcggtgcGACGAGCGCGGCGAT | |
| AttB 46 | ATCATCATCCATGGtgtatcattttcatatagttagcacctgcacactatatgaaa | |
| N675015_95_5 | atgatacaTGAGCTGCGAGAA | |
| integrase | | |
| ACTB N-term | GCTATTCTCGCAGCTCACCAgttttagagctagaaatagcaagttaaaataaggct | 310 |
| PBS 13 RT 29 | agtccgttatcaacttgaaaaagtggcaccgagtcggtgcGACGAGCGCGGCGAT | |
| AttB 46 | ATCATCATCCATGGtgtctactatctgtatatgcgacacatgtggcataaagaca | |
| N189929_49_54 | tagtagacaTGAGCTGCGAGAA | |
| integrase | | |
| ACTB N-term | GCTATTCTCGCAGCTCACCAgttttagagctagaaatagcaagttaaaataaggct | 311 |
| PBS 13 RT 29 | agtccgttatcaacttgaaaaagtggcaccgagtcggtgcGACGAGCGCGGCGAT | |
| AttB 46 | ATCATCATCCATGGcatcgaccctgacgcatgcggaggcggcgctccatgcgtc | |
| N203911_45186_ | tgacctcattTGAGCTGCGAGAA | |
| integrase | | |
| ACTB N-term | GCTATTCTCGCAGCTCACCAgttttagagctagaaatagcaagttaaaataaggct | 312 |
| PBS 13 RT 29 | agtccgttatcaacttgaaaaagtggcaccgagtcggtgcGACGAGCGCGGCGAT | |
| AttB 46 | ATCATCATCCATGGgttagtacccaaatgacaaaaggtcatccttttatcatttgg | |
| N687663_53_ | gtactaacTGAGCTGCGAGAA | |
| 29 | | |
| integrase | | |
| ACTB N-term | GCTATTCTCGCAGCTCACCAgttttagagctagaaatagcaagttaaaataaggct | 313 |
| PBS 13 RT 29 | agtccgttatcaacttgaaaaagtggcaccgagtcggtgcGACGAGCGCGGCGAT | |
| AttB 46 | ATCATCATCCATGGcttattaaaacccgttccgcttctgtcaaagcggcatcggtt | |
| N687611_90_ | ttataaacTGAGCTGCGAGAA | |
| 68 | | |
| integrase | | |
| ACTB N-term | GCTATTCTCGCAGCTCACCAgttttagagctagaaatagcaagttaaaataaggct | 314 |
| PBS 13 RT 29 | agtccgttatcaacttgaaaaagtggcaccgagtcggtgcGACGAGCGCGGCGAT | |
| AttB 46 | ATCATCATCCATGGggcgtgatggtcgtgaacctcaacatgacgacgaacacg | |
| N190156_234_ | acctcgcggccTGAGCTGCGAGAA | |
| 12 integrase | | |
| ACTB N-term | GCTATTCTCGCAGCTCACCAgttttagagctagaaatagcaagttaaaataaggct | 315 |
| PBS 13 RT 29 | agtccgttatcaacttgaaaaagtggcaccgagtcggtgcGACGAGCGCGGCGAT | |
| AttB 46 | ATCATCATCCATGGtctacatcttgaatatatcaagttataactttgaattatatca | |
| N191533_224_ | gtttataTGAGCTGCGAGAA | |
| 76 integrase | | |
| ACTB N-term | GCTATTCTCGCAGCTCACCAgttttagagctagaaatagcaagttaaaataaggct | 316 |
| PBS 13 RT 29 | agtccgttatcaacttgaaaaagtggcaccgagtcggtgcGACGAGCGCGGCGAT | |
| AttB 46 | ATCATCATCCATGGaattatatctaaaagcactaagctccgccatactgctttta | |
| N208621_9_15 | gatataataTGAGCTGCGAGAA | |
| integrase | | |
| ACTB N-term | GCTATTCTCGCAGCTCACCAgttttagagctagaaatagcaagttaaaataaggct | 317 |
| PBS 13 RT 29 | agtccgttatcaacttgaaaaagtggcaccgagtcggtgcGACGAGCGCGGCGAT | |
| AttB 46 | ATCATCATCCATGGgatatggggaagtgaatcagtacaaccgccacagtaccT | |
| Bacillus_cereus_ | GAGCTGCGAGAA | |
| AH187_38 | | |
| bp_Att | | |
| ACTB N-term | GCTATTCTCGCAGCTCACCAgttttagagctagaaatagcaagttaaaataaggct | 318 |
| PBS 13 RT 29 | agtccgttatcaacttgaaaaagtggcaccgagtcggtgcGACGAGCGCGGCGAT | |
| AttB 46 | ATCATCATCCATGGggtactgtggcggttgtactgattcacttccccatatcTGA | |
| Bacillus_cereus_ | GCTGCGAGAA | |
| AH187_38 | | |
| bp_Att_rc | | |
| ACTB N-term | GCTATTCTCGCAGCTCACCAgttttagagctagaaatagcaagttaaaataaggct | 319 |
| PBS 13 RT 29 | agtccgttatcaacttgaaaaagtggcaccgagtcggtgcGACGAGCGCGGCGAT | |
| AttB 46 | ATCATCATCCATGGtgggtggtacaggtgccacattagttgtaccatttatgTG | |
| Staphylococcus_ | AGCTGCGAGAA | |
| lugdunensis_ | | |
| N920143_38 bp_ | | |
| Att | | |
| ACTB N-term | GCTATTCTCGCAGCTCACCAgttttagagctagaaatagcaagttaaaataaggct | 320 |
| PBS 13 RT 29 | agtccgttatcaacttgaaaaagtggcaccgagtcggtgcGACGAGCGCGGCGAT | |
| AttB 46 | ATCATCATCCATGGcataaatggtacaactaatgtggcacctgtaccacccaT | |
| Staphylococcus_ | GAGCTGCGAGAA | |
| lugdunensis_ | | |
| N920143_38 bp_ | | |
| Att_rc | | |
| ACTB N-term | GCTATTCTCGCAGCTCACCAgttttagagctagaaatagcaagttaaaataaggct | 321 |
| PBS 13 RT 29 | agtccgttatcaacttgaaaaagtggcaccgagtcggtgcGACGAGCGCGGCGAT | |
| AttB 46 | ATCATCATCCATGGgttgtttttccagatccagttggtcctgtaaatataagTGA | |
| Bacillus_ | GCTGCGAGAA | |
| cytotoxicus_ | | |
| NVH_391- | | |
| 98_38 bp_Att | | |
| ACTB N-term | GCTATTCTCGCAGCTCACCAgttttagagctagaaatagcaagttaaaataaggct | 322 |
| PBS 13 RT 29 | agtccgttatcaacttgaaaaagtggcaccgagtcggtgcGACGAGCGCGGCGAT | |
| AttB 46 | ATCATCATCCATGGcttatatttacaggaccaactggatctggaaaaacaacT | |
| Bacillus_ | GAGCTGCGAGAA | |
| cytotoxicus_ | | |
| NVH_391- | | |
| 98_38 bp_Att_rc | | |
| ACTB N-term | GCTATTCTCGCAGCTCACCAgttttagagctagaaatagcaagttaaaataaggct | 323 |
| PBS 13 RT 29 | agtccgttatcaacttgaaaaagtggcaccgagtcggtgcGACGAGCGCGGCGAT | |
| AttB 46 | ATCATCATCCATGGgtactgtggcggttgtactgattcacttccccatatTGAG | |
| Bacillus_cereus_ | CTGCGAGAA | |
| AH187_Att | | |
| 36 bp | | |
| ACTB N-term | GCTATTCTCGCAGCTCACCAgttttagagctagaaatagcaagttaaaataaggct | 324 |
| PBS 13 RT 29 | agtccgttatcaacttgaaaaagtggcaccgagtcggtgcGACGAGCGCGGCGAT | |
| AttB 46 | ATCATCATCCATGGtactgtggcggttgtactgattcacttccccataTGAGC | |
| Bacillus_cereus_ | TGCGAGAA | |
| AH187_Att | | |
| 34 bp | | |
| ACTB N-term | GCTATTCTCGCAGCTCACCAgttttagagctagaaatagcaagttaaaataaggct | 325 |
| PBS 13 RT 29 | agtccgttatcaacttgaaaaagtggcaccgagtcggtgcGACGAGCGCGGCGAT | |
| AttB 46 | ATCATCATCCATGGactgtggcggttgtactgattcacttccccatTGAGCTG | |
| Bacillus_cereus_ | CGAGAA | |
| AH187_Att | | |
| 32 bp | | |
| ACTB N-term | GCTATTCTCGCAGCTCACCAgttttagagctagaaatagcaagttaaaataaggct | 326 |
| PBS 13 RT 29 | agtccgttatcaacttgaaaaagtggcaccgagtcggtgcGACGAGCGCGGCGAT | |
| AttB 46 | ATCATCATCCATGGatatggggaagtgaatcagtacaaccgccacagtacTG | |
| Bacillus_cereus_ | AGCTGCGAGAA | |
| AH187_Att_ | | |
| rc 36 bp | | |
| ACTB N-term | GCTATTCTCGCAGCTCACCAgttttagagctagaaatagcaagttaaaataaggct | 327 |
| PBS 13 RT 29 | agtccgttatcaacttgaaaaagtggcaccgagtcggtgcGACGAGCGCGGCGAT | |
| AttB 46 | ATCATCATCCATGGtatggggaagtgaatcagtacaaccgccacagtaTGAG | |
| Bacillus_cereus_ | CTGCGAGAA | |
| AH187_Att_ | | |
| rc 34 bp | | |
| ACTB N-term | GCTATTCTCGCAGCTCACCAgttttagagctagaaatagcaagttaaaataaggct | 328 |
| PBS 13 RT 29 | agtccgttatcaacttgaaaaagtggcaccgagtcggtgcGACGAGCGCGGCGAT | |
| AttB 46 | ATCATCATCCATGGatggggaagtgaatcagtacaaccgccacagtTGAGC | |
| Bacillus_cereus_ | TGCGAGAA | |
| AH187_Att_ | | |
| rc 32 bp | | |
| ACTB N-term | GCTATTCTCGCAGCTCACCAgttttagagctagaaatagcaagttaaaataaggct | 329 |
| PBS 13 RT 29 | agtccgttatcaacttgaaaaagtggcaccgagtcggtgcGACGAGCGCGGCGAT | |
| AttB 46 | ATCATCATCCATGGataaatggtacaactaatgtggcacctgtaccacccTGA | |
| Staphylococcus_ | GCTGCGAGAA | |
| lugdunensis_ | | |
| N920143_Att | | |
| 36 bp | | |
| ACTB N-term | GCTATTCTCGCAGCTCACCAgttttagagctagaaatagcaagttaaaataaggct | 330 |
| PBS 13 RT 29 | agtccgttatcaacttgaaaaagtggcaccgagtcggtgcGACGAGCGCGGCGAT | |
| AttB 46 | ATCATCATCCATGGtaaatggtacaactaatgtggcacctgtaccaccTGAG | |
| Staphylococcus_ | CTGCGAGAA | |
| lugdunensis_ | | |
| N920143_Att | | |
| 34 bp | | |
| ACTB N-term | GCTATTCTCGCAGCTCACCAgttttagagctagaaatagcaagttaaaataaggct | 331 |
| PBS 13 RT 29 | agtccgttatcaacttgaaaaagtggcaccgagtcggtgcGACGAGCGCGGCGAT | |
| AttB 46 | ATCATCATCCATGGaaatggtacaactaatgtggcacctgtaccacTGAGCT | |
| Staphylococcus_ | GCGAGAA | |
| lugdunensis_ | | |
| N920143_Att | | |
| 32 bp | | |
| ACTB N-term | GCTATTCTCGCAGCTCACCAgttttagagctagaaatagcaagttaaaataaggct | 332 |
| PBS 13 RT 29 | agtccgttatcaacttgaaaaagtggcaccgagtcggtgcGACGAGCGCGGCGAT | |
| AttB 46 | ATCATCATCCATGGgggtggtacaggtgccacattagttgtaccatttatTGA | |
| Staphylococcus_ | GCTGCGAGAA | |
| lugdunensis_ | | |
| N920143_Att | | |
| rc 36 bp | | |
| ACTB N-term | GCTATTCTCGCAGCTCACCAgttttagagctagaaatagcaagttaaaataaggct | 333 |
| PBS 13 RT 29 | agtccgttatcaacttgaaaaagtggcaccgagtcggtgcGACGAGCGCGGCGAT | |
| AttB 46 | ATCATCATCCATGGggtggtacaggtgccacattagttgtaccatttaTGAGC | |
| Staphylococcus_ | TGCGAGAA | |
| lugdunensis_ | | |
| N920143_Att | | |
| rc 34 bp | | |
| ACTB N-term | GCTATTCTCGCAGCTCACCAgttttagagctagaaatagcaagttaaaataaggct | 334 |
| PBS 13 RT 29 | agtccgttatcaacttgaaaaagtggcaccgagtcggtgcGACGAGCGCGGCGAT | |
| AttB 46 | ATCATCATCCATGGgtggtacaggtgccacattagttgtaccatttTGAGCT | |
| Staphylococcus_ | GCGAGAA | |
| lugdunensis_ | | |
| N920143_Att | | |
| rc 32 bp | | |
| ACTB N-term | GCTATTCTCGCAGCTCACCAgttttagagctagaaatagcaagttaaaataaggct | 335 |
| PBS 13 RT 29 | agtccgttatcaacttgaaaaagtggcaccgagtcggtgcGACGAGCGCGGCGAT | |
| AttB 46 | ATCATCATCCATGGttatatttacaggaccaactggatctggaaaaacaaTGA | |
| Bacillus_ | GCTGCGAGAA | |
| cytotoxicus_NVH_ | | |
| 391-98_Att | | |
| 36 bp | | |
| ACTB N-term | GCTATTCTCGCAGCTCACCAgttttagagctagaaatagcaagttaaaataaggct | 336 |
| PBS 13 RT 29 | agtccgttatcaacttgaaaaagtggcaccgagtcggtgcGACGAGCGCGGCGAT | |
| AttB 46 | ATCATCATCCATGGtatatttacaggaccaactggatctggaaaaacaTGAG | |
| Bacillus_ | CTGCGAGAA | |
| cytotoxicus_ | | |
| NVH_ | | |
| 391-98_Att | | |
| 34 bp | | |
| ACTB N-term | GCTATTCTCGCAGCTCACCAgttttagagctagaaatagcaagttaaaataaggct | 337 |
| PBS 13 RT 29 | agtccgttatcaacttgaaaaagtggcaccgagtcggtgcGACGAGCGCGGCGAT | |
| AttB 46 | ATCATCATCCATGGatatttacaggaccaactggatctggaaaaacTGAGC | |
| Bacillus_ | TGCGAGAA | |
| cytotoxicus_NVH_ | | |
| 391-98_Att | | |
| 32 bp | | |
| ACTB N-term | GCTATTCTCGCAGCTCACCAgttttagagctagaaatagcaagttaaaataaggct | 338 |
| PBS 13 RT 29 | agtccgttatcaacttgaaaaagtggcaccgagtcggtgcGACGAGCGCGGCGAT | |
| AttB 46 | ATCATCATCCATGGttgtttttccagatccagttggtcctgtaaatataaTGAG | |
| Bacillus_ | CTGCGAGAA | |
| cytotoxicus_NVH_ | | |
| 391-98_Att_rc | | |
| 36 bp | | |
| ACTB N-term | GCTATTCTCGCAGCTCACCAgttttagagctagaaatagcaagttaaaataaggct | 339 |
| PBS 13 RT 29 | agtccgttatcaacttgaaaaagtggcaccgagtcggtgcGACGAGCGCGGCGAT | |
| AttB 46 | ATCATCATCCATGGtgtttttccagatccagttggtcctgtaaatataTGAGCT | |
| Bacillus_ | GCGAGAA | |
| cytotoxicus_NVH_ | | |
| 391-98_Att_rc | | |
| 34 bp | | |
| ACTB N-term | GCTATTCTCGCAGCTCACCAgttttagagctagaaatagcaagttaaaataaggct | 340 |
| PBS 13 RT 29 | agtccgttatcaacttgaaaaagtggcaccgagtcggtgcGACGAGCGCGGCGAT | |
| AttB 46 | ATCATCATCCATGGgtttttccagatccagttggtcctgtaaatatTGAGCTG | |
| Bacillus_ | CGAGAA | |
| cytotoxicus_NVH_ | | |
| 391-98_Att_rc | | |
| 32 bp | | |
| Bacillus_cereus_ | GCTGTCTCCGCCGCCCGCCAgttttagagctagaaatagcaagttaaaataaggc | 341 |
| AH187_Att | tagtccgttatcaacttgaaaaagtggcaccgagtcggtgcAGTCGCCATGatatgggg | |
| rc 36 LMNB1 | aagtgaatcagtacaaccgccacagtacCGGGCGGCG | |
| PBS 9 RT | | |
| 10_AttB 36 | | |
| atgRNA | | |
| Bacillus_cereus_ | GCGTATTGCCTGGAGGATGGgttttagagctagaaatagcaagttaaaataaggc | 342 |
| AH187_Att_ | tagtccgttatcaacttgaaaaagtggcaccgagtcggtgcGAACCACGCGGCGA | |
| rc_36 NOLC1 | ATGCCGGCGTCCGCCatatggggaagtgaatcagtacaaccgccacagtacT | |
| PBS 18 RT | CCTCCAGGCAATACGCG | |
| 29_AttB 36 | | |
| atgRNA | | |
| Bacillus_cereus_ | GAGAAGCGGCGTCCGGGGCTAgttttagagctagaaatagcaagttaaaataag | 343 |
| AH187_Att_ | gctagtccgttatcaacttgaaaaagtggcaccgagtcggtgcTCTTTGTCCAGAGT | |
| rc_36 | CACAGCCATAatatggggaagtgaatcagtacaaccgccacagtacCCCCGG | |
| SUPT16H PBS | ACGCCGC | |
| 13 | | |
| RT 24_AttB 36 | | |
| atgRNA | | |
| Bacillus_cereus_ | GGGCACGGGGCCATGTACAAgttttagagctagaaatagcaagttaaaataagg | 344 |
| AH187_Att_ | ctagtccgttatcaacttgaaaaagtggcaccgagtcggtgcGGCGTCGGCAGCCC | |
| rc_36 SRRM2 | GATCCCGTTGatatggggaagtgaatcagtacaaccgccacagtacTACATGG | |
| PBS 13 RT | CCCCGT | |
| 24_AttB 36 | | |
| atgRNA | | |
| Bacillus_cereus_ | GTGTCAGGTGGGGCGGGGCTAgttttagagctagaaatagcaagttaaaataag | 345 |
| AH187_Att_ | gctagtccgttatcaacttgaaaaagtggcaccgagtcggtgcGCTGGCTCCTCCCC | |
| rc_36 | TGGCACCATAatatggggaagtgaatcagtacaaccgccacagtacCCCCGC | |
| DEPDC4 PBS | CCCACCTGACAC | |
| 18 | | |
| RT 24_AttB 36 | | |
| atgRNA | | |
| Bacillus_cereus_ | GAGTGGGTCAGACGAGCAGGAgttttagagctagaaatagcaagttaaaataa | 346 |
| AH187_Att_ | ggctagtccgttatcaacttgaaaaagtggcaccgagtcggtgcCGACTCCTCCCCC | |
| rc_ 36 NES | ATGCAGCCCTCCATCatatggggaagtgaatcagtacaaccgccacagtacT | |
| PBS 13 RT 28 | GCTCGTCTGACC | |
| AttB 36 | | |
| atgRNA | | |
| B. cereus_ | GCTGTCTCCGCCGCCCGCCAgttttagagctagaaatagcaagttaaaataaggc | 347 |
| LMNB1_PBS 9 | tagtccgttatca | |
| RT 20_AttB 36 | acttgaaaaagtggcaccgagtcggtgcCGGGGGTCGCAGTCGCCATGata | |
| atgRNA | tggggaagtgaatcagtacaaccgccacagtacCGGGCGGCG | |
| B. cereus_ | GGGCACGGGGCCATGTACAAgttttagagctagaaatagcaagttaaaataagg | 348 |
| LMNB1_PBS | ctagtccgttatcaacttgaaaaagtggcaccgagtcggtgcCGGGGGTCGCAGTCG | |
| 13 RT 20_AttB | CCATGatatggggaagtgaatcagtacaaccgccacagtacCGGGCGGCGGA | |
| 36 atgRNA | GA | |
| B. cereus_ | GGGCACGGGGCCATGTACAAgttttagagctagaaatagcaagttaaaataagg | 349 |
| LMNB1_PBS | ctagtccgttatcaacttgaaaaagtggcaccgagtcggtgcGCGGCGGCACGGGG | |
| 13 RT 29_AttB | GTCGCAGTCGCCATGatatggggaagtgaatcagtacaaccgccacagtacC | |
| 36 atgRNA | GGGCGGCGGAGA | |
| B. cereus_ | GCGTATTGCCTGGAGGATGGgttttagagctagaaatagcaagttaaaataaggc | 350 |
| NOLC1_PBS | tagtccgttatcaacttgaaaaagtggcaccgagtcggtgcGAACCACGCGGCGAA | |
| 13 RT 29_AttB | TGCCGGCGTCCGCCatatggggaagtgaatcagtacaaccgccacagtacTCC | |
| 36 atgRNA | TCCAGGCAAT | |
| B. cereus_ | GGGCACGGGGCCATGTACAAgttttagagctagaaatagcaagttaaaataagg | 351 |
| NOLC1_PBS | ctagtccgttatcaacttgaaaaagtggcaccgagtcggtgcGGCGAATGCCGGCG | |
| 13 RT 20_AttB | TCCGCCatatggggaagtgaatcagtacaaccgccacagtacTCCTCCAGGCA | |
| 36 atgRNA | AT | |
| B. cereus_ | GGGCACGGGGCCATGTACAAgttttagagctagaaatagcaagttaaaataagg | 352 |
| NOLC1_PBS | ctagtccgttatcaacttgaaaaagtggcaccgagtcggtgcGGCGAATGCCGGCG | |
| 18 RT 20_AttB | TCCGCCatatggggaagtgaatcagtacaaccgccacagtacTCCTCCAGGCA | |
| 36 atgRNA | ATACGCG | |
| B. cereus_ | GGGCACGGGGCCATGTACAAgttttagagctagaaatagcaagttaaaataagg | 353 |
| SRRM2_PBS 9 | ctagtccgttatcaacttgaaaaagtggcaccgagtcggtgcGGCGTCGGCAGCCC | |
| RT 24_AttB 36 | GATCCCGTTGatatggggaagtgaatcagtacaaccgccacagtacTACATGG | |
| atgRNA | CC | |
| B. cereus_ | GGGCACGGGGCCATGTACAAgttttagagctagaaatagcaagttaaaataagg | 354 |
| SRRM2_PBS 9 | ctagtccgttatcaacttgaaaaagtggcaccgagtcggtgcGATCCCGTTGatatggg | |
| RT 10_AttB 36 | gaagtgaatcagtacaaccgccacagtacTACATGGCC | |
| atgRNA | | |
| B. cereus_ | GGGCACGGGGCCATGTACAAgttttagagctagaaatagcaagttaaaataagg | 355 |
| SRRM2_PBS | ctagtccgttatcaacttgaaaaagtggcaccgagtcggtgcGATCCCGTTGatatggg | |
| 13 RT 10_AttB | gaagtgaatcagtacaaccgccacagtacTACATGGCCCCGT | |
| 36 atgRNA | | |
| Screen | GCTATTCTCGCAGCTCACCAgttttagagctagaaatagcaagttaaaataaggct | 356 |
| validation | agtccgttatcaacttgaaaaagtggcaccgagtcggtgcgcgcggcgatatcatcatccatggat | |
| guides | gatcctgacgacggagaccgccgtcgtcgacaagcctgagctgcgag | |
| ACTB_1_11_24_ | | |
| 38 | | |
| Screen | GCTATTCTCGCAGCTCACCAgttttagagctagaaatagcaagttaaaataaggct | 357 |
| validation | agtccgttatcaacttgaaaaagtggcaccgagtcggtgccgatatcatcatccatggoggatgatc | |
| guides | ctgacgacggagaccgccgtcgtcgacaagccggctgagctgcgagaatag | |
| ACTB_1_16_18_ | | |
| 43 | | |
| Screen | GCTGTCTCCGCCGCCCGCCAgttttagagctagaaatagcaagttaaaataaggc | 358 |
| validation | tagtccgttatcaacttgaaaaagtggcaccgagtcggtgcgcggcacgggggtcgcagtcgcca | |
| guides | tgatgatcctgacgacggagaccgccgtcgtcgacaagcccgggcggc | |
| LMNB1_1_8_26_ | | |
| 38 | | |
| Screen | GCGTATTGCCTGGAGGATGGgttttagagctagaaatagcaagttaaaataaggc | 359 |
| validation | tagtccgttatcaacttgaaaaagtggcaccgagtcggtgcaatgccggcgtccgcccggatgatc | |
| guides | ctgacgacggagaccgccgtcgtcgacaagccggctcctccaggcaatac | |
| NOLC1_1_15_ | | |
| 16_43 | | |
| Screen | GCGTATTGCCTGGAGGATGGgttttagagctagaaatagcaagttaaaataaggc | 360 |
| validation | tagtccgttatcaacttgaaaaagtggcaccgagtcggtgcggcgtccgccatgatcctgacgacg | |
| guides | gagaccgccgtcgtcgacaagcctcctccaggcaata | |
| NOLC1_1_14_ | | |
| 10_38 | | |
| Screen | GGGAAATGCATCTTGCACAAgttttagagctagaaatagcaagttaaaataagg | 361 |
| validation | ctagtccgttatcaacttgaaaaagtggcaccgagtcggtgcagcccctccatgctctctagctgttg | |
| guides | ccattgggcttgtcgacgacggcggtctccgtcgtcaggatcattgcaagatgcatt | |
| SERPIN_13_32_ | | |
| 38 | | |
| Screen | GTGTCAGGTGGGGCGGGGCTAgttttagagctagaaatagcaagttaaaataag | 362 |
| validation | gctagtccgttatcaacttgaaaaagtggcaccgagtcggtgctggcaccataatgatcctgacgac | |
| guides | ggagaccgccgtcgtcgacaagccccccgccc | |
| DEPDC4_8_10_ | | |
| 38 | | |
| SERPIN | GTGGGGACAGCCCCGTCTCTgttttagagctagaaatagcaagttaaaataaggc | 363 |
| Nicking guide - | tagtccgttatcaacttgaaaaagtggcaccgagtcggtgc | |
| 107 guide | | |
| SERPIN | GCTCTTGGGAAAAAAACCCTAgttttagagctagaaatagcaagttaaaataag | 364 |
| Nicking guide - | gctagtccgttatcaacttgaaaaagtggcaccgagtcggtgc | |
| 91 guide | | |
| SERPIN | GTCTTGGGAAAAAAACCCTAAgttttagagctagaaatagcaagttaaaataag | 365 |
| Nicking guide - | gctagtccgttatcaacttgaaaaagtggcaccgagtcggtgc | |
| 90 guide | | |
| SERPIN | GAAAAAAACCCTAAGGGCTGgttttagagctagaaatagcaagttaaaataagg | 366 |
| Nicking guide - | ctagtccgttatcaacttgaaaaagtggcaccgagtcggtgc | |
| 84 guide | | |
| SERPIN | GCTGAGGATCCTTGTGAGTGTgttttagagctagaaatagcaagttaaaataag | 367 |
| Nicking guide - | gctagtccgttatcaacttgaaaaagtggcaccgagtcggtgc | |
| 67 guide | | |
| SERPIN | GTGAGGATCCTTGTGAGTGTTgttttagagctagaaatagcaagttaaaataagg | 368 |
| Nicking guide - | ctagtccgttatcaacttgaaaaagtggcaccgagtcggtgc | |
| 66 guide | | |
| SERPIN | GGATCCTTGTGAGTGTTGGGgttttagagctagaaatagcaagttaaaataaggc | 369 |
| Nicking guide - | tagtccgttatcaacttgaaaaagtggcaccgagtcggtgc | |
| 63 guide | | |
| SERPIN | GATCCTTGTGAGTGTTGGGTgttttagagctagaaatagcaagttaaaataaggct | 370 |
| Nicking guide - | agtccgttatcaacttgaaaaagtggcaccgagtcggtgc | |
| 62 guide | | |
| SERPIN | GTTGGGTGGGAACAGCTCCCgttttagagctagaaatagcaagttaaaataaggc | 371 |
| Nicking guide - | tagtccgttatcaacttgaaaaagtggcaccgagtcggtgc | |
| 49 guide | | |
| SERPIN | GGGTGGGAACAGCTCCCAGGgttttagagctagaaatagcaagttaaaataagg | 372 |
| Nicking guide - | ctagtccgttatcaacttgaaaaagtggcaccgagtcggtgc | |
| 46 guide | | |
| SERPIN | GCTTCTGTGCAGCAGTTTCCCgttttagagctagaaatagcaagttaaaataagg | 373 |
| Nicking guide | ctagtccgttatc aacttgaaaaagtggcaccgagtcggtgc | |
| +34 guide | | |
| SERPIN | GTTTCCCTGGCCACTAAATAGgttttagagctagaaatagcaagttaaaataagg | 374 |
| Nicking guide | ctagtccgttatc aacttgaaaaagtggcaccgagtcggtgc | |
| +48 guide | | |
| SERPIN | GTTCCCTGGCCACTAAATAGTgttttagagctagaaatagcaagttaaaataagg | 375 |
| Nicking guide | ctagtccgttatc aacttgaaaaagtggcaccgagtcggtgc | |
| +49 guide | | |
| SERPIN | GATTAGATAGAAGCCCTCCAgttttagagctagaaatagcaagttaaaataaggc | 376 |
| Nicking guide | tagtccgttatca acttgaaaaagtggcaccgagtcggtgc | |
| +71 guide | | |
| SERPIN | GATTAGATAGAAGCCCTCCAAgttttagagctagaaatagcaagttaaaataag | 377 |
| Nicking guide | gctagtccgttat caacttgaaaaagtggcaccgagtcggtgc | |
| +72 guide | | |

### 6.8. Integrases/Recombinases and Integration/Recombination Sites

In typical embodiments, the co-delivery system described herein contains an integrase and/or a recombinase. In some embodiments, the co-delivery system includes an integrase and/or a recombinase packaged in a LNP. In one embodiment, the co-delivery system includes a polynucleotide encoding an integrase and/or a recombinase. In some embodiments, the co-delivery system includes an integrase or a recombinase packaged in a vector (e.g., a viral vector). In some embodiments, the co-delivery system includes at least a first integrase (e.g., a first integrase and a second integrase) and/or at least a first recombinase (e.g., a first recombinase and a second recombinase).

In some embodiments, the integration enzyme (e.g., the integrase or recombinase) is selected from the group consisting of Dre, Vika, Bxb1, φC31, RDF, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by a Tc1/mariner family member including but not limited to retrotransposases encoded by LI, Tol2, Tel, Tc3, Himar 1 (isolated from the horn fly, Haematobia irritans), Mos1 (Mosaic element of Drosophila mauritiana), and Minos, and any mutants thereof. As can be used herein, Xu et al describes methods for evaluating integrase activity in E. coli and mammalian cells and confirmed at least R4, φC31, φBT1, Bxb1, SPBc, TP901-1 and Wβ integrases to be active on substrates integrated into the genome of HT1080 cells (Xu et al., 2013, Accuracy and efficiency define Bxb1 integrase as the best of fifteen candidate serine recombinases for the integration of DNA into the human genome. BMC Biotechnol. 2013 Oct. 20; 13:87. doi: 10.1186/1472-6750-13-87). Durrant describes new large serine recombinases (LSRs) divided into three classes distinguished from one another by efficiency and specificity, including landing pad LSRs which outperform wild-type Bxb1 in episomal and chromosomal integration efficiency, LSRs that achieve both efficient and site-specific integration without a landing pad, and multi-targeting LSRs with minimal site-specificity. Additionally, embodiments can include any serine recombinase such as BceINT, SSCINT, SACINT, and INT10 (see Ionnidi et al., 2021; Drag-and-drop genome insertion without DNA cleavage with CRISPR directed integrases. bioRxiv 2021.11.01.466786, doi.org/10.1101/2021.11.01.466786). In some embodiments, the integration site can be selected from an attB site, an attP site, an attL site, an attR site, a lox71 site a Vox site, or a FRT site.

It will be appreciated that desired activity of integrases, transposases and the like can depend on nuclear localization. In certain embodiments, prokaryotic enzymes are adapted to modulate nuclear localization. In certain embodiments, eukaryotic or vertebrate enzymes are adapted to modulate nuclear localization. In certain embodiments, the invention provides fusion or hybrid proteins. Such modulation can comprise addition or removal of one or more nuclear localization signal (NLS) and/or addition or removal of one or more nuclear export signal (NES). Xu et al compared derivatives of fourteen serine integrases that either possess or lack a nuclear localization signal (NLS) to conclude that certain integrases benefit from addition of an NLS whereas others are transported efficiently without addition, and a major determinant of activity in yeast and vertebrate cells is avoidance of toxicity. (Xu et al., 2016, Comparison and optimization of ten phage encoded serine integrases for genome engineering in Saccharomyces cerevisiae. BMC Biotechnol. 2016 Feb. 9; 16:13. doi: 10.1186/s12896-016-0241-5). Ramakrishnan et al. systematically studied the effect of different NES mutants developed from mariner-like elements (MLEs) on transposase localization and activity and concluded that nuclear export provides a means of controlling transposition activity and maintaining genome integrity. (Ramakrishnan et al. Nuclear export signal (NES) of transposases affects the transposition activity of mariner-like elements Ppmar1 and Ppmar2 of moso bamboo. Mob DNA. 2019 Aug. 19; 10:35. doi:10.1186/s13100-019-0179-y). The methods and constructs are used to modulate nuclear localization of system components of the invention.

In typical embodiments, the integrase used herein is selected from below (Table 10).

TABLE 10
Integrases

| | | | | protein | | | | | | | | | |
|---|---|---|---|---|---|---|---|---|---|---|---|---|---|
| | | | nucleo- | accession | | | | | | | | | |
| | | | tide | or | internal | Pro- | Alter- | | | | SEQ | | |
| Data- | SRA | bioproject_ | acces- | ORF | protein | posed | native | organism/ | de- | | ID | | |
| base | accession | acc | sion | ID | ID | names | names | source | scription | Sequence | NO: | Length | Group |
| ENA | SRS1205298 | PRJEB26277 | NA | NA | N189929_ | SsuINT | NA | human | stool | MEKNRAVLYLRLSKEDVDKV | 378 | 527 | INT |
| | | | | | 49_54 | | | gut | sample | NKGDDSSSIKSQRLLLTDFALE | | | c |
| | | | | | | | | metagenome | from male | RGFKIVGVYSDDDESGLYDDR | | | |
| | | | | | | | | | in USA | PDFERMMTDAKLDEFDIIIAKT | | | |
| | | | | | | | | | | QSRFSRNMEHIEKYLHHDLPN | | | |
| | | | | | | | | | | LGIRFIGAVDGVDTESDENKKS | | | |
| | | | | | | | | | | RQINGLVNEWYCEDLSKNIRS | | | |
| | | | | | | | | | | AFKAKMKDGQFLGSSCPYGY | | | |
| | | | | | | | | | | KKDPQNHNHLVVDDYAAKVV | | | |
| | | | | | | | | | | QKIFNLYLEGYGKAKIGSILSSE | | | |
| | | | | | | | | | | GILIPTLYKKDILKQNYHNSKA | | | |
| | | | | | | | | | | LDTTQNWSYQTIHTILNNEVY | | | |
| | | | | | | | | | | LGHLIQNKVNTMSYKDKNKRI | | | |
| | | | | | | | | | | LPKEKWIIVRNTHEPIITEEMFQ | | | |
| | | | | | | | | | | DVQKLQKNRTRSVENIEPNGL | | | |
| | | | | | | | | | | FSGLIFCADCKHAMSRKYARR | | | |
| | | | | | | | | | | GEKGFVGYVCKTYKTQGKNF | | | |
| | | | | | | | | | | CESHSIDYDELEEAVLFSIKNE | | | |
| | | | | | | | | | | ARSILQQEEIDELRKVQAYDET | | | |
| | | | | | | | | | | KSYYEMQLENIKSRMEKIEKY | | | |
| | | | | | | | | | | KKKTYDNYMDDLISRDDYKK | | | |
| | | | | | | | | | | YVTEYDKEIGGLKQQQELINS | | | |
| | | | | | | | | | | KTDLEKEISTQYDEWVEAFINY | | | |
| | | | | | | | | | | VDIDKLTREIVIELIEKIEVNKD | | | |
| | | | | | | | | | | GSINIYYKFKNPYIS | | | |
| ENA | ERS396461 | PRJEB26280 | NA | NA | N190156_ | SssINT | NA | human | stool | MNTVIYARYSAGPRQTDQSID | 379 | 510 | INT |
| | | | | | 234_12 | | | gut | sample | GQLRVCTEFCKQRGLTVVDTY | | | d |
| | | | | | | | | metagenome | from Spain | CDRHISGRTDERPEFQRLIADA | | | |
| | | | | | | | | | | KAHKFEAVVVYKTDRFARNK | | | |
| | | | | | | | | | | YDSAIYKRELRRNGIQIFYAAE | | | |
| | | | | | | | | | | AIPEGPEGIILESLMEGLAEYYS | | | |
| | | | | | | | | | | AELAQKIKRGLNESALKCQSL | | | |
| | | | | | | | | | | GSGRPLGYTVDEQKHFQIDPES | | | |
| | | | | | | | | | | SQAVKTIFEMYIKGESNAAICD | | | |
| | | | | | | | | | | YLNARGLRTSQGNLFNKNSIN | | | |
| | | | | | | | | | | RIIKNRKYIGEYRYNDIVVEGG | | | |
| | | | | | | | | | | MPAIISKETFCMAQAEMERRR | | | |
| | | | | | | | | | | THRAPVSPKAEYLLAGKLFCG | | | |
| | | | | | | | | | | HCKGPMQGVSGTGKSGNKWY | | | |
| | | | | | | | | | | YYYCANTRGKERTCDKKQVS | | | |
| | | | | | | | | | | RDRLEKAVVDFTVRYILQENV | | | |
| | | | | | | | | | | LEELSKKVYAAQERQNNTASE | | | |
| | | | | | | | | | | IAFYEKKLAENKKAIANILRAI | | | |
| | | | | | | | | | | ESGAMTQALPARLQELENEQT | | | |
| | | | | | | | | | | VIQGELSYLKGARLAFTEDQIL | | | |
| | | | | | | | | | | FALLQHLDPRPGESERDYHRRI | | | |
| | | | | | | | | | | ITDFVSEVYLYDDRMLIYFNIS | | | |
| | | | | | | | | | | SADGKLKHADLSAIESGVFDA | | | |
| | | | | | | | | | | GLISSSSRASSFSTRCALI | | | |
| ENA | ERS1015837 | PRJEB26832 | NA | NA | N191352_ | SscINT | NA | human | stool | MNEKNLEIGAAYIRVSTDDQT | 380 | 482 | INT |
| | | | | | 143_72 | | | gut | sample | ELSPDAQLRVILEAAKKDGIIIP | | | d |
| | | | | | | | | metagenome | from China | QEFVFMEDRGRSGRRADNRPE | | | |
| | | | | | | | | | | FQRMISTARQNPSPFRYLYLW | | | |
| | | | | | | | | | | KFSRFARNQEESAFYKGILRKK | | | |
| | | | | | | | | | | CGVTIKSVSEPIMEGMFGRLVE | | | |
| | | | | | | | | | | MIIEWSDEFYSVNLSGEVLRG | | | |
| | | | | | | | | | | MTQKALEHGYQLTPCLGYDA | | | |
| | | | | | | | | | | VGHGRPYVINEEQYQIVEFIHR | | | |
| | | | | | | | | | | SFFDGKDMTWIAREANRRGY | | | |
| | | | | | | | | | | HTRRGNPFDTRAVRIILTNSFY | | | |
| | | | | | | | | | | VGLVKWNDVTFQGTHECRES | | | |
| | | | | | | | | | | VTSVFSANQERLNRIHRPRGRR | | | |
| | | | | | | | | | | QASSCKHWLSGLLKCSICGAS | | | |
| | | | | | | | | | | LGYNQTKDLTKRGHAFQCWK | | | |
| | | | | | | | | | | YTKGIHPGSCSVSSLKAEAAVL | | | |
| | | | | | | | | | | ESLQMILETGEVEYTYEQREK | | | |
| | | | | | | | | | | HLDDNKLTLIQKSLERLDTKEL | | | |
| | | | | | | | | | | RIREAYESGIDTLDEFKTNKAR | | | |
| | | | | | | | | | | LQRERDQLMEELEELHSQEEP | | | |
| | | | | | | | | | | EDVPGKEILIERIQNVYDLLQSP | | | |
| | | | | | | | | | | DVDNDDKGNAVRSIIKKIVYIK | | | |
| | | | | | | | | | | ESKTFCFYYYV | | | |
| ENA | ERS1289677 | PRJEB26924 | NA | NA | N191533_ | Ssc2INT | NA | human | stool | MERTIKVIQPGTVKIPTKKRVA | 381 | 406 | INT |
| | | | | | 224_76 | | | gut | sample | AYARVSSGKDAMLHSLSAQVS | | | c |
| | | | | | | | | metagenome | from China | YYSNMIQQKNEWSYVGIYADE | | | |
| | | | | | | | | | | AITGTKDRRVEFNRLIQDCTDG | | | |
| | | | | | | | | | | KIDMIITKSISRFARNTLTMLEV | | | |
| | | | | | | | | | | VRKLKNINVDVYFEKENIHSIS | | | |
| | | | | | | | | | | GDGELMLTILASFAQEESRSVS | | | |
| | | | | | | | | | | ENCKWRIRKGFEQGELINLRFL | | | |
| | | | | | | | | | | YGYRINKGKIEIYEKEAEIVRM | | | |
| | | | | | | | | | | IFDDYLNGEGCTRIGNKLRKM | | | |
| | | | | | | | | | | KVNKLRGGMWNSERVVDIIK | | | |
| | | | | | | | | | | NEKYTGNALLQKKYVKDHLS | | | |
| | | | | | | | | | | KKLVRNKGILTQYYAEGTHPA | | | |
| | | | | | | | | | | IIDIKTFEIAQKIMEANRTKFQG | | | |
| | | | | | | | | | | KCGSNRYLFTSKIECGICGKNY | | | |
| | | | | | | | | | | RHKDREGKSTWVCANHLKYG | | | |
| | | | | | | | | | | NSRCIAKPLNEEKLKKLINEAL | | | |
| | | | | | | | | | | ELKYFDEEIFIRNIKRIKVTGNQ | | | |
| | | | | | | | | | | TIEFILKDGKVIEEGMI | | | |
| ENA | ERS2655827 | PRJEB28245 | NA | NA | N203911_ | SsdINT | NA | human | stool | MKKIKIDRAIQERPATRKQTRN | 382 | 401 | INT |
| | | | | | 45186_6 | | | gut | sample | EKIRQSLTEHVDVQVIPAITDR | | | c |
| | | | | | | | | metagenome | from | EGYEKPKLRVCAYCRVSTDM | | | |
| | | | | | | | | | Denmark | DTQALSYELQVQNYTDYIRGN | | | |
| | | | | | | | | | | DEWRFAGIYADRGISGTSLKH | | | |
| | | | | | | | | | | RDEFNRMIEDCKAGKIDLIITK | | | |
| | | | | | | | | | | AVTRFARNVLDCISTIRMLKQL | | | |
| | | | | | | | | | | EHPVAVYFETERINTLDTTSET | | | |
| | | | | | | | | | | YLGLISLFAQGESESKSESLKW | | | |
| | | | | | | | | | | SYIRRWKRGTGIYPAWSLLGY | | | |
| | | | | | | | | | | EMGEDGKWQIVEAEAELVRII | | | |
| | | | | | | | | | | YDMYLNGYSSPQIAEILTRSGV | | | |
| | | | | | | | | | | PTATNQTVWSSGGVLGILRNE | | | |
| | | | | | | | | | | KYCGNVLCQKTMTVDVFSHK | | | |
| | | | | | | | | | | AIKNTGQKTQYFIEGHHDPIILR | | | |
| | | | | | | | | | | SDWDRVQQMIDEKYYRKRRG | | | |
| | | | | | | | | | | RRTKPRIVLKGCLAGFTQIDLD | | | |
| | | | | | | | | | | WDEDDIARIFYSTTPAAEVATP | | | |
| | | | | | | | | | | AMADHIEIIKVKGEN | | | |
| ENA | SRS294942 | PRJEB30046 | NA | NA | N208621_ | SmcINT | NA | human | sample | MKTAAAYIRVSTDDQVEYSPD | 383 | 476 | INT |
| | | | | | 9_15 | | | gut | from 72- | SQIKLIRDYAKRNDYILPDEFIF | | | d |
| | | | | | | | | metagenome | year-old | RDDGISGKSAKHRPEFTKMIAL | | | |
| | | | | | | | | | male from | AKSPEHPFDAILVWKFSRFARN | | | |
| | | | | | | | | | China | QEESIVFKNILRKIGVEVRSVSE | | | |
| | | | | | | | | | | PISEDPFGSLVERIIEWTDEYYI | | | |
| | | | | | | | | | | INLSGEVKRGMLEKISRGQPVV | | | |
| | | | | | | | | | | PPPVGYKMENGQYIPDENAHFI | | | |
| | | | | | | | | | | KEIFEAYAAGEGARHIAQRLA | | | |
| | | | | | | | | | | AQGCLTKRGNPIDNRFVDYVL | | | |
| | | | | | | | | | | HNPVYIGKLRWSVNSHAASSR | | | |
| | | | | | | | | | | HYDSADIIVFDGTHEPLISSEL | | | |
| | | | | | | | | | | WESVQKRLHEVKTLYPKYQR | | | |
| | | | | | | | | | | REQPVSFMLKGLVRCSSCGST | | | |
| | | | | | | | | | | LCYCRTSEPSLQCHSYARGSCR | | | |
| | | | | | | | | | | QSHSINIATANEAVIKGLQLAV | | | |
| | | | | | | | | | | DKLDFAIAPAKPHYSADAPGT | | | |
| | | | | | | | | | | NKLLAAEYKKMERIKAAYAN | | | |
| | | | | | | | | | | GTDTLEEYAANKKKISAEIARL | | | |
| | | | | | | | | | | EAELQQESNVKPINKKAFAKR | | | |
| | | | | | | | | | | VSEIIKYISDPHNSEAAKNQAL | | | |
| | | | | | | | | | | RTVISYIIFDRAATTFNIIFHF | | | |
| MetaSUB | NA | NA | NA | NA | N675015_ | UhmINT | NA | urban | NA | MKIAIYARKSKYSPTGESVENQ | 384 | 550 | INT |
| | | | | | 95_5 | | | human | | IQLCKEYLQAKYKSETLEIDEY | | | d |
| | | | | | | | | microbiome | | KDEGYSGGNTNRPDFKKLIAQI | | | |
| | | | | | | | | | | EDYDMLICYRLDRISRNVADFS | | | |
| | | | | | | | | | | STLTLLQNNKCDFVSIKEQFDT | | | |
| | | | | | | | | | | TSPMGRAMIYISSVFAQLERET | | | |
| | | | | | | | | | | IAERIRDNMMELAKMGRWLG | | | |
| | | | | | | | | | | GTIPMGFDSEPITFIDENMKERS | | | |
| | | | | | | | | | | MTKLIPNVEELKVIELIYEKYL | | | |
| | | | | | | | | | | QLGSMGKVVTYLLQNNIKTKK | | | |
| | | | | | | | | | | GKDFTLGSIKVILTNPIYVKAN | | | |
| | | | | | | | | | | QEVVNHLKTQGITICGDVDGK | | | |
| | | | | | | | | | | KALLTYNKTTGISNDVGTKTIV | | | |
| | | | | | | | | | | KDKSEWIAAVANHKGIIPADK | | | |
| | | | | | | | | | | WLQAQNIKDKNKDSFPALGRS | | | |
| | | | | | | | | | | NTTIASRVLRCDKCESTMGVT | | | |
| | | | | | | | | | | HGHINPVTGKKHYYYNCTLKK | | | |
| | | | | | | | | | | RSKGVRCDNKPAKAAEVDEAI | | | |
| | | | | | | | | | | LITLENMFKAKSSIIDNLKAKN | | | |
| | | | | | | | | | | KARRIEMISSNRVDVINKIIEDK | | | |
| | | | | | | | | | | TKQIDNLVNKLSLDDDLTDILF | | | |
| | | | | | | | | | | KKIKGLKAEIKELEDELLTLTS | | | |
| | | | | | | | | | | DNIKLNEDEVVLDFTEKLLEKC | | | |
| | | | | | | | | | | SIIRTLDILEQQQIVDALIPLVT | | | |
| | | | | | | | | | | WNGDTEVLNIYPLGSPELELKE | | | |
| | | | | | | | | | | AESKKK | | | |
| Segata- | NA | PRJNA422434 | NA | NA | N684346_ | SacINT | NA | human | stool | MKEKVSERKTGAIYIRVSTDK | 385 | 493 | INT |
| Pasolli | | | | | 90_69 | | | gut | sample | QEELSPDAQLRLLLDYAKKDSI | | | d |
| | | | | | | | | metagenome | from adult | DVPKEYIFQDNGISGRKANKRP | | | |
| | | | | | | | | | in China | AFQNMIALAKSKEHPIDTIIVW | | | |
| | | | | | | | | | | KFSRFARNQEESIVYKSLLKKN | | | |
| | | | | | | | | | | NVDVVSVSEPLIDGPFGSLIERI | | | |
| | | | | | | | | | | IEWMDEYYSIRLSGEVMRGMT | | | |
| | | | | | | | | | | QNAMRGHYQSDAPIGYTSPGD | | | |
| | | | | | | | | | | KKPPVINPDTVQIPLMIKDMFL | | | |
| | | | | | | | | | | SGSTQLQIARKLNDSGYRTKR | | | |
| | | | | | | | | | | GNLWDARGVRYVLENPFYIGK | | | |
| | | | | | | | | | | SRWNYTERGRRLKPADEVIYA | | | |
| | | | | | | | | | | DGNWEALWDEDTFKEIQKRL | | | |
| | | | | | | | | | | ALNMRKSKSRDISAAKHWLSG | | | |
| | | | | | | | | | | LLICSSCGGTLAFGGAHNMRG | | | |
| | | | | | | | | | | FQCWKYSKGFCSESHYISTGPI | | | |
| | | | | | | | | | | EKMVLEYLEAVMHSPALSYTV | | | |
| | | | | | | | | | | ISSSSVDASSKLSDLERQLQKID | | | |
| | | | | | | | | | | AKEKRIKAAYLNEIDTLEEYK | | | |
| | | | | | | | | | | ANKTALEEERRTVEKEIEELTL | | | |
| | | | | | | | | | | SDVKYSKEDLDKKMKQNISDL | | | |
| | | | | | | | | | | LRVLRDESADYIQKGNMMRN | | | |
| | | | | | | | | | | VVDHIVFNRKNTSLDVFLKLVV | | | |
| Segata- | ERR1136864 | PRJEB11532 | NA | NA | N687611_ | RsaINT | NA | human | rectal swab | MKITKKQPLRPRGRSEDKRQS | 386 | 404 | INT |
| PasoLi | | | | | 90_68 | | | gut | from adult | TKNVIRDAYINGPQKEVQIIPA | | | c |
| | | | | | | | | metagenome | in Isreal | KRDMEAETEKKKLRVCAYCR | | | |
| | | | | | | | | | | VSTDEDTQASSYELQVQNYTR | | | |
| | | | | | | | | | | MIRENPEWEFAGIFADEGISGT | | | |
| | | | | | | | | | | SVLHREHFLEMIEKCKAGEIDL | | | |
| | | | | | | | | | | IITKQVSRFARNVLDSLNYIFM | | | |
| | | | | | | | | | | LRKLDPPVGVYFETEKLNTLD | | | |
| | | | | | | | | | | KSSDMVITVLSLVAQSESEQKS | | | |
| | | | | | | | | | | NSLKWSFKRRRAQGLGIYPSW | | | |
| | | | | | | | | | | ALLGYRLDDEKNWEIVEDEAD | | | |
| | | | | | | | | | | IVRTIYSLYLDGYSSTQIAELLT | | | |
| | | | | | | | | | | KSGIPTVKGLSVWSSGSVLGIL | | | |
| | | | | | | | | | | KNEKFCGDALCQKTVTIDFFT | | | |
| | | | | | | | | | | HKSVKNNGIEPQYFVEGHHIPII | | | |
| | | | | | | | | | | EKNDWLLAQQIRKERRYRKRR | | | |
| | | | | | | | | | | STHRKPRIVVKGALSGFMIVDT | | | |
| | | | | | | | | | | SWDEEYVDSLLISATQKPEPAP | | | |
| | | | | | | | | | | VIAEEDENFIVIEKE | | | |
| Segata- | ERR1136737 | PRJEB11532 | NA | NA | N687663_ | Rsa2INT | NA | human | rectal swab | MADIQPVKNGALYIRVSTHLQ | 387 | 498 | INT |
| Pasolli | | | | | 53_29 | | | gut | from adult | EELSPDAQKRLLMEYAEAHNII | | | d |
| | | | | | | | | metagenome | in Isreal | VLKEHIYIDSGISGRSARQRPQF | | | |
| | | | | | | | | | | NNMIAEAKSKEHPFDVILVWK | | | |
| | | | | | | | | | | YSRFARNQEESIVYKSMLKRE | | | |
| | | | | | | | | | | NVDVISVSEPISDDPFGSLIERI | | | |
| | | | | | | | | | | IEWMDEYYSIRLSGEVSRGMAE | | | |
| | | | | | | | | | | NAMRGNYQARPPLGYRIPGYR | | | |
| | | | | | | | | | | QTPVIVPEEAELIQLIFDLYTEK | | | |
| | | | | | | | | | | KMGIFEIVRYLNEHGYQTGHK | | | |
| | | | | | | | | | | KPFQRRSVTYILKNPTYIGKTI | | | |
| | | | | | | | | | | WNQHDQDHKLRDKSEWIIAD | | | |
| | | | | | | | | | | GKHEPIISKEQFDKAQKRIEST | | | |
| | | | | | | | | | | YKPAYRKPTSVCHHWLSSLLK | | | |
| | | | | | | | | | | CSSCGRTLVVKRTASKKKDRM | | | |
| | | | | | | | | | | YVNFQCYGYQKGICNTNQSIS | | | |
| | | | | | | | | | | AIKLEPVIMHALEDAMTSGKIH | | | |
| | | | | | | | | | | FDVLNPTTLDSSQKQQFLTRLN | | | |
| | | | | | | | | | | EIEKKEERIKRAYRDGIDTLEE | | | |
| | | | | | | | | | | YKENKSIIQTEKEMLLKKIEHIE | | | |
| | | | | | | | | | | EPALSPEEAKPIMMDRIKNVYE | | | |
| | | | | | | | | | | IITNPDIGMEEKNKAARSIIEKI | | | |
| | | | | | | | | | | VFDRATGSVNIFFYLAHCP | | | |
| NCBI | NA | NA | NC_ | NP_ | NA | BxbINT | Bxb1 | Mycobacterium | NA | MRALVVIRLSRVTDATTSPER | 388 | 501 | INT |
| | | | 002656.1 | 75302.1 | | | integrase | phage | | QLESCQQLCAQRGWDVVGVA | | | a |
| | | | | | | | | Bxb1 | | EDLDVSGAVDPFDRKRRPNLA | | | |
| | | | | | | | | | | RWLAFEEQPFDVIVAYRVDRL | | | |
| | | | | | | | | | | TRSIRHLQQLVHWAEDHKKLV | | | |
| | | | | | | | | | | VSATEAHFDTTTPFAAVVIAL | | | |
| | | | | | | | | | | MGTVAQMELEAIKERNRSAA | | | |
| | | | | | | | | | | HFNIRAGKYRGSLPPWGYLPT | | | |
| | | | | | | | | | | RVDGEWRLVPDPVQRERILEV | | | |
| | | | | | | | | | | YHRVVDNHEPLHLVAHDLNR | | | |
| | | | | | | | | | | RGVLSPKDYFAQLQGREPQGR | | | |
| | | | | | | | | | | EWSATALKRSMISEAMLGYAT | | | |
| | | | | | | | | | | LNGKTVRDDDGAPLVRAEPIL | | | |
| | | | | | | | | | | TREQLEALRAELVKTSRAKPA | | | |
| | | | | | | | | | | VSTPSLLLRVLFCAVCGEPAYK | | | |
| | | | | | | | | | | FAGGGRKHPRYRCRSMGFPKH | | | |
| | | | | | | | | | | CGNGTVAMAEWDAFCEEQVL | | | |
| | | | | | | | | | | DLLGDAERLEKVWVAGSDSA | | | |
| | | | | | | | | | | VELAEVNAELVDLTSLIGSPAY | | | |
| | | | | | | | | | | RAGSPQREALDARIAALAARQ | | | |
| | | | | | | | | | | EELEGLEARPSGWEWRETGQR | | | |
| | | | | | | | | | | FGDWWREQDTAAKNTWLRS | | | |
| | | | | | | | | | | MNVRLTFDVRGGLTRTIDFGD | | | |
| | | | | | | | | | | LQEYEQHLRLGSVVERL | | | |
| | | | | | | | | | | HTGMS* | | | |
| NCBI | NA | NA | NC_ | NP_ | NA | Tp9INT | TP901-1 | Lactococcus | NA | MTKKVAIYTRVSTTNQAEEGF | 389 | 486 | INT |
| | | | 002747.1 | 112664.1 | | | integrase | phage | | SIDEQIDRLTKYAEAMGWQVS | | | d |
| | | | | | | | | TP901-1 | | DTYTDAGFSGAKLERPAMQRL | | | |
| | | | | | | | | | | INDIENKAFDTVLVYKLDRLSR | | | |
| | | | | | | | | | | SVRDTLYLVKDVFTKNKIDFIS | | | |
| | | | | | | | | | | LNESIDTSSAMGSLFLTILSAIN | | | |
| | | | | | | | | | | EFERENIKERMTMGKLGRAKS | | | |
| | | | | | | | | | | GKSMMWTKTAFGYYHNRKTG | | | |
| | | | | | | | | | | ILEIVPLQATIVEQIFTDYLSGI | | | |
| | | | | | | | | | | SLTKLRDKLNESGHIGKDIPWS | | | |
| | | | | | | | | | | YRTLRQTLDNPVYCGYIKFKD | | | |
| | | | | | | | | | | SLFEGMHKPIIPYETYLKVQKE | | | |
| | | | | | | | | | | LEERQQQTYERNNNPRPFQAK | | | |
| | | | | | | | | | | YMLSGMARCGYCGAPLKIVL | | | |
| | | | | | | | | | | GHKRKDGSRTMKYHCANRFP | | | |
| | | | | | | | | | | RKTKGITVYNDNKKCDSGTYD | | | |
| | | | | | | | | | | LSNLENTVIDNLIGFQENNDSL | | | |
| | | | | | | | | | | LKIINGNNQPILDTSSFKKQISQ | | | |
| | | | | | | | | | | IDKKIQKNSDLYLNDFITMDEL | | | |
| | | | | | | | | | | KDRTDSLQAEKKLLKAKISEN | | | |
| | | | | | | | | | | KFNDSTDVFELVKTQLGSIPIN | | | |
| | | | | | | | | | | ELSYDNKKKIVNNLVSKVDVT | | | |
| | | | | | | | | | | ADNVDIIFKFQLA* | | | |
| NCBI | NA | NA | NC_ | NP_ | NA | Bt1INT | PhiBT | Streptomyces | NA | MSPFIAPDVPEHLLDTVRVFLY | 390 | 595 | INT |
| | | | 004664.2 | 813744.2 | | | integrase | virus | | ARQSKGRSDGSDVSTEAQLAA | | | a |
| | | | | | | | | phiBT1 | | GRALVASRNAQGGARWVVAG | | | |
| | | | | | | | | | | EFVDVGRSGWDPNVTRADFER | | | |
| | | | | | | | | | | MMGEVRAGEGDVVVVNELSR | | | |
| | | | | | | | | | | LTRKGAHDALEIDNELKKHGV | | | |
| | | | | | | | | | | RFMSVLEPFLDTSTPIGVAIFAL | | | |
| | | | | | | | | | | IAALAKQDSDLKAERLKGAKD | | | |
| | | | | | | | | | | EIAALGGVHSSSAPFGMRAVR | | | |
| | | | | | | | | | | KKVDNLVISVLEPDEDNPDHV | | | |
| | | | | | | | | | | ELVERMAKMSFEGVSDNAIAT | | | |
| | | | | | | | | | | TFEKEKIPSPGMAERRATEKRL | | | |
| | | | | | | | | | | ASIKARRLNGAEKPIMWRAQT | | | |
| | | | | | | | | | | VRWILNHPAIGGFAFERVKHG | | | |
| | | | | | | | | | | KAHINVIRRDPGGKPLTPHTGI | | | |
| | | | | | | | | | | LSGSKWLELQEKRSGKNLSDR | | | |
| | | | | | | | | | | KPGAEVEPTLLSGWRFLGCRIC | | | |
| | | | | | | | | | | GGSMGQSQGGRKRNGDLAEG | | | |
| | | | | | | | | | | NYMCANPKGHGGLSVKRSEL | | | |
| | | | | | | | | | | DEFVASKVWARLRTADMEDE | | | |
| | | | | | | | | | | HDQAWIAAAAERFALQHDLA | | | |
| | | | | | | | | | | GVADERREQQAHLDNVRRSIK | | | |
| | | | | | | | | | | DLQADRKAGLYVGREELETW | | | |
| | | | | | | | | | | RSTVLQYRSYEAECTTRLAEL | | | |
| | | | | | | | | | | DEKMNGSTRVPSEWFSGEDPT | | | |
| | | | | | | | | | | AEGGIWASWDVYERREFLSFF | | | |
| | | | | | | | | | | LDSVMVDRGRHPETKKYIPLK | | | |
| | | | | | | | | | | DRVTLKWAELLKEEDEASEAT | | | |
| | | | | | | | | | | ERELAAL* | | | |
| NCBI | NA | NA | NC_ | WP_ | NA | BceINT | NA | Bacillus | NA | MYPYDVPDYAGSYRPESLDVC | 391 | 529 | INT |
| | | | 011658.1 | 000286206.1 | | | | cereus | | IYLRKSRKDVEEERRAIEEGSS | | | c |
| | | | | | | | | AH187 | | YNALERHRKRLFAIAKAENHN | | | |
| | | | | | | | | | | IIDIFEEVASGESIQERPQMQQL | | | |
| | | | | | | | | | | LRKLEGNEIDGVLVIDLDRLGR | | | |
| | | | | | | | | | | GDMLDAGMIDRAFRYSSTKIIT | | | |
| | | | | | | | | | | PTDVYDPDDESWELVFGIKSLI | | | |
| | | | | | | | | | | SRQELKSITKRLQNGRIDSVKE | | | |
| | | | | | | | | | | GKHIGKKPPYGYLKDENLRLY | | | |
| | | | | | | | | | | PDPEKAWIVKKIFELMCDGKG | | | |
| | | | | | | | | | | RQMIAAELDRLGIDPPVTKRG | | | |
| | | | | | | | | | | AWDSSTITSIIKNEVYTGVIVW | | | |
| | | | | | | | | | | GKFKHKKRNGKYTRHKNPQE | | | |
| | | | | | | | | | | KWIMYENAHEPIISKELFDAAN | | | |
| | | | | | | | | | | EAHSSRHKPAVITSKKLTNPLA | | | |
| | | | | | | | | | | GILKCKLCGYTMLIQTRKDRP | | | |
| | | | | | | | | | | HNYLRCNNPACKGKQKQSVF | | | |
| | | | | | | | | | | NLVEEKLLYSLQQIVDEYQAQ | | | |
| | | | | | | | | | | KVEEVEIDDSKLISFKEKAIISK | | | |
| | | | | | | | | | | EKELKELQAQKGNLHDLLEQG | | | |
| | | | | | | | | | | IYTVEIFLERQKNLVERITSIEN | | | |
| | | | | | | | | | | DIEVLQKEIETEQIKEHNKTEFI | | | |
| | | | | | | | | | | PALKTVIESYHKTTNIELKNQL | | | |
| | | | | | | | | | | LKTILSTVTYYRHPDWKTNEF | | | |
| | | | | | | | | | | EIQVYFKIS* | | | |
| NCBI | NA | NA | NC_ | WP_ | NA | BcyINT | NA | Bacillus | NA | MYPYDVPDYAGSAVGIYIRVS | 392 | 487 | INT |
| | | | 009674.1 | 012095429.1 | | | | cytotoxicus | | TQEQASEGHSIESQKKKLASYC | | | d |
| | | | | | | | | NVH391-98 | | EIQGWDDYRFYIEEGISGKNTN | | | |
| | | | | | | | | | | RPKLKLLMEHIEKGKINILLVY | | | |
| | | | | | | | | | | RLDRLTRSVIDLHKLLNFLQEH | | | |
| | | | | | | | | | | GCAFKSATETYDTTTANGRMS | | | |
| | | | | | | | | | | MGIVSLLAQWETENMSERIKL | | | |
| | | | | | | | | | | NLEHKVLVEGERVGAIPYGFD | | | |
| | | | | | | | | | | LSDDEKLVKNEKSAILLDMVE | | | |
| | | | | | | | | | | RVENGWSVNRIVNYLNLTNN | | | |
| | | | | | | | | | | DRNWSPNGVLRLLRNPALYG | | | |
| | | | | | | | | | | ATRWNDKIAENTHEGIISKERF | | | |
| | | | | | | | | | | NRLQQILADRSIHHRRDVKGT | | | |
| | | | | | | | | | | YIFQGVLRCPVCDQTLSVNRFI | | | |
| | | | | | | | | | | KKRKDGTEYCGVLYRCQPCIK | | | |
| | | | | | | | | | | QNKYNLAIGEARFLKALNEYM | | | |
| | | | | | | | | | | STVEFQTVEDEVIPKKSEREML | | | |
| | | | | | | | | | | ESQLQQIARKREKYQKAWASD | | | |
| | | | | | | | | | | LMSDDEFEKLMVETRETYDEC | | | |
| | | | | | | | | | | KQKLESCEDPIKIDETYLKEIV | | | |
| | | | | | | | | | | YMFHQTFNDLESEKQKEFISKF | | | |
| | | | | | | | | | | IRTIRYTVKEQQPIRPDKSKTG | | | |
| | | | | | | | | | | KGKQKVIITEVE FYQS* | | | |
| NCBI | NA | NA | NC_ | WP_ | NA | SluINT | NA | Staphylococcus | NA | MYPYDVPDYAGSKVAIYTRVS | 393 | 473 | INT |
| | | | 017353.1 | 014533238.1 | | | | lugdunensis | | SAEQANEGYSIHEQKKKLISYC | | | d |
| | | | | | | | | N920143 | | EIHDWNEYKVFTDAGISGGSM | | | |
| | | | | | | | | | | KRPALQKLMKHLSSFDLVLVY | | | |
| | | | | | | | | | | KLDRLTRNVRDLLDMLEEFEQ | | | |
| | | | | | | | | | | YNVSFKSATEVFDTTSAIGKLF | | | |
| | | | | | | | | | | ITMVGAMAEWERETIRERSLF | | | |
| | | | | | | | | | | GSRAAVREGNYIREAPFCYDNI | | | |
| | | | | | | | | | | EGKLHPNEYAKVIDLIVSMFK | | | |
| | | | | | | | | | | KGISANEIARRLNSSKVHVPNK | | | |
| | | | | | | | | | | KSWNRNSLIRLMRSPVLRGHT | | | |
| | | | | | | | | | | KYGDMLIENTHEPVLSEHDYN | | | |
| | | | | | | | | | | AINNAISSKTHKSKVKHHAIFR | | | |
| | | | | | | | | | | GALVCPQCNRRLHLYAGTVK | | | |
| | | | | | | | | | | DRKGYKYDVRRYKCETCSKN | | | |
| | | | | | | | | | | KDVKNVSFNESEVENKFVNLL | | | |
| | | | | | | | | | | KSYELNKFHIRKVEPVKKIEYD | | | |
| | | | | | | | | | | IDKINKQKINYTRSWSLGYIED | | | |
| | | | | | | | | | | DEYFELMEEINATKKMIEEQTT | | | |
| | | | | | | | | | | ENKQSVSKEQIQSINNFILKGWE | | | |
| | | | | | | | | | | ELTIKDKEELILSTVDKIEFNFI | | | |
| | | | | | | | | | | PKDKKHK TNTLDINNIHFKFS* | | | |

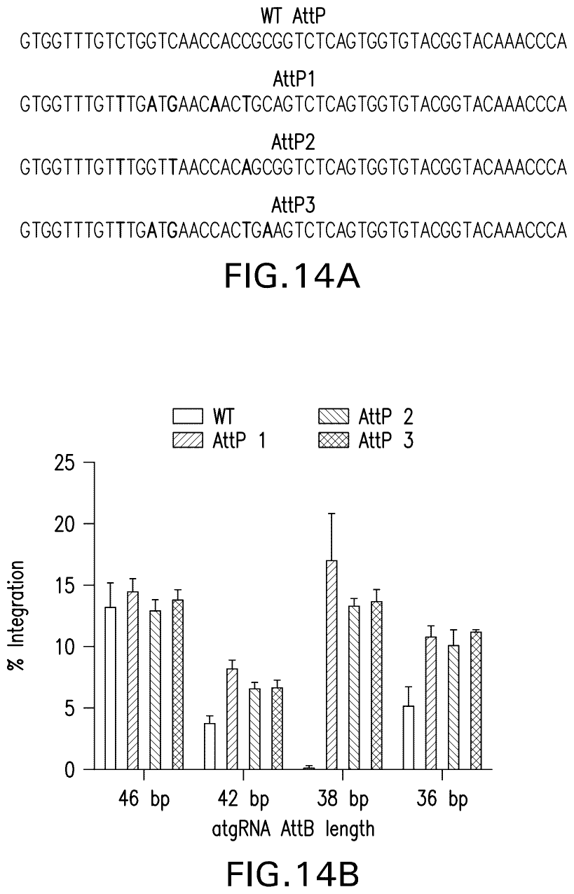

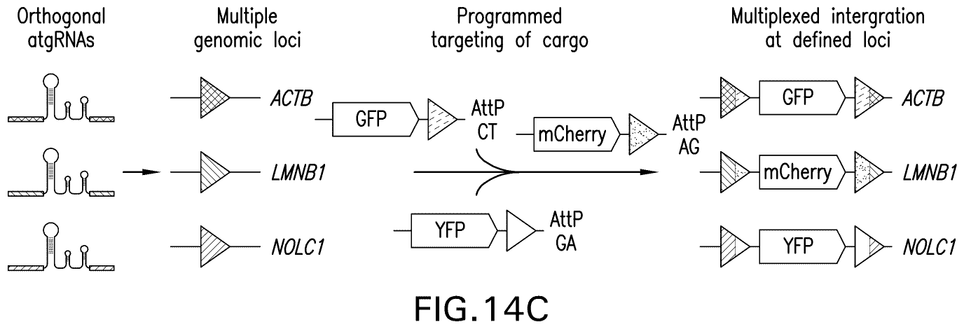

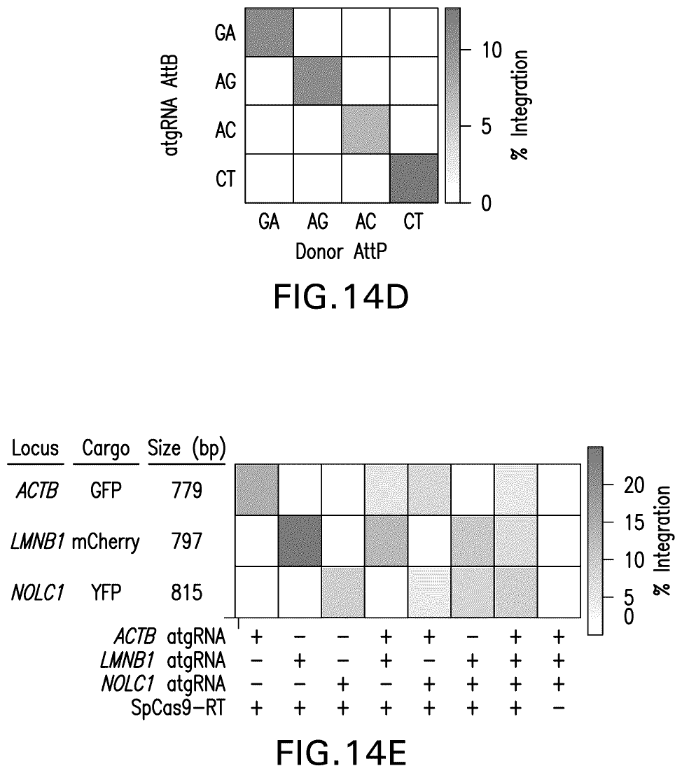

Sequences of insertion sites (i.e., recognition target sites) suitable for use in embodiments of the disclosure are presented below (Table 11). FIGS. 14A-14E shows analysis of effect of variant AttP sites on integration efficiency.

TABLE 11

| | Forward Sequence | SEQ ID | Reverse Sequence | SEQ ID |
|---|---|---|---|---|
| Description | (5′-3′) | NO: | (5′-3′) | NO: |
| Bxb1_AttP_ | GTGGTTTGTCTGGTCAAC | SEQ ID | TGGGTTTGTACCGTACACCACTGAGA | SEQ ID |
| GT_original_ | CACCGCGGTCTCAGTGGT | NO: | CCGCGGTGGTTGACCAGACAAACCAC | NO: |
| site | GTACGGTACAAACCCA | 394 | | 473 |
| Bxb1_AttP_ | GTGGTTTGTCTGGTCAAC | 395 | TGGGTTTGTACCGTACACCACTGAGC | 474 |
| CG_site | CACCGCGcgCTCAGTGGTG | | GCGCGGTGGTTGACCAGACAAACCAC | |
| | TACGGTACAAACCCA | | | |
| Bxb1_AttP_ | GTGGTTTGTCTGGTCAAC | 396 | TGGGTTTGTACCGTACACCACTGAGG | 475 |
| GC_site | CACCGCGgcCTCAGTGGTG | | CCGCGGTGGTTGACCAGACAAACCAC | |
| | TACGGTACAAACCCA | | | |
| Bxb1_AttP_ | GTGGTTTGTCTGGTCAAC | 397 | TGGGTTTGTACCGTACACCACTGAGA | 476 |
| AT_site | CACCGCGatCTCAGTGGTG | | TCGCGGTGGTTGACCAGACAAACCAC | |
| | TACGGTACAAACCCA | | | |
| Bxb1_AttP_ | GTGGTTTGTCTGGTCAAC | 398 | TGGGTTTGTACCGTACACCACTGAGT | 477 |
| TA_site | CACCGCGtaCTCAGTGGTG | | ACGCGGTGGTTGACCAGACAAACCAC | |
| | TACGGTACAAACCCA | | | |
| Bxb1_AttP_ | GTGGTTTGTCTGGTCAAC | 399 | TGGGTTTGTACCGTACACCACTGAGC | 478 |
| GG_site | CACCGCGggCTCAGTGGTG | | CCGCGGTGGTTGACCAGACAAACCAC | |
| | TACGGTACAAACCCA | | | |
| Bxb1_AttP_ | GTGGTTTGTCTGGTCAAC | 400 | TGGGTTTGTACCGTACACCACTGAGA | 479 |
| TT_site | CACCGCGttCTCAGTGGTG | | ACGCGGTGGTTGACCAGACAAACCAC | |
| | TACGGTACAAACCCA | | | |
| Bxb1_AttP_ | GTGGTTTGTCTGGTCAAC | 401 | TGGGTTTGTACCGTACACCACTGAGT | 480 |
| GA_site | CACCGCGgaCTCAGTGGTG | | CCGCGGTGGTTGACCAGACAAACCAC | |
| | TACGGTACAAACCCA | | | |
| Bxb1_AttP_ | GTGGTTTGTCTGGTCAAC | 402 | TGGGTTTGTACCGTACACCACTGAGC | 481 |
| AG_site | CACCGCGagCTCAGTGGTG | | TCGCGGTGGTTGACCAGACAAACCAC | |
| | TACGGTACAAACCCA | | | |
| Bxb1_AttP_ | GTGGTTTGTCTGGTCAAC | 403 | TGGGTTTGTACCGTACACCACTGAGG | 482 |
| CC_site | CACCGCGccCTCAGTGGTG | | GCGCGGTGGTTGACCAGACAAACCAC | |
| | TACGGTACAAACCCA | | | |
| Bxb1_AttP_ | GTGGTTTGTCTGGTCAAC | 404 | TGGGTTTGTACCGTACACCACTGAGG | 483 |
| TC_site | CACCGCGtcCTCAGTGGTG | | ACGCGGTGGTTGACCAGACAAACCAC | |
| | TACGGTACAAACCCA | | | |
| Bxb1_AttP_ | GTGGTTTGTCTGGTCAAC | 405 | TGGGTTTGTACCGTACACCACTGAGA | 484 |
| CT_site | CACCGCGctCTCAGTGGTG | | GCGCGGTGGTTGACCAGACAAACCAC | |
| | TACGGTACAAACCCA | | | |
| Bxb1_AttP_ | GTGGTTTGTCTGGTCAAC | 406 | TGGGTTTGTACCGTACACCACTGAGT | 485 |
| AA_site | CACCGCGaaCTCAGTGGTG | | TCGCGGTGGTTGACCAGACAAACCAC | |
| | TACGGTACAAACCCA | | | |
| Bxb1_AttP_ | GTGGTTTGTCTGGTCAAC | 407 | TGGGTTTGTACCGTACACCACTGAGT | 486 |
| CA_site | CACCGCGcaCTCAGTGGTG | | GCGCGGTGGTTGACCAGACAAACCAC | |
| | TACGGTACAAACCCA | | | |
| Bxb1_AttP_ | GTGGTTTGTCTGGTCAAC | 408 | TGGGTTTGTACCGTACACCACTGAGG | 487 |
| AC_site | CACCGCGacCTCAGTGGTG | | TCGCGGTGGTTGACCAGACAAACCAC | |
| | TACGGTACAAACCCA | | | |
| Bxb1_AttP_ | GTGGTTTGTCTGGTCAAC | 409 | TGGGTTTGTACCGTACACCACTGAGC | 488 |
| TG_site | CACCGCGtgCTCAGTGGTG | | ACGCGGTGGTTGACCAGACAAACCAC | |
| | TACGGTACAAACCCA | | | |
| Bxb1_AttB_ | GGCCGGCTTGTCGACGAC | 410 | CCGGATGATCCTGACGACGGAGACCG | 489 |
| 46_GT_ | GGCGGTCTCCGTCGTCAG | | CCGTCGTCGACAAGCCGGCC | |
| original_site | GATCATCCGG | | | |
| Bxb1_AttB_ | GGCCGGCTTGTCGACGAC | 411 | CCGGATGATCCTGACGACGGAGTTCG | 490 |
| 46_AA_site | GGCGaaCTCCGTCGTCAGG | | CCGTCGTCGACAAGCCGGCC | |
| | ATCATCCGG | | | |
| Bxb1_AttB_ | GGCCGGCTTGTCGACGAC | 412 | CCGGATGATCCTGACGACGGAGTCCG | 491 |
| 46_GA_site | GGCGgaCTCCGTCGTCAGG | | CCGTCGTCGACAAGCCGGCC | |
| | ATCATCCGG | | | |
| Bxb1_AttB_ | GGCCGGCTTGTCGACGAC | 413 | CCGGATGATCCTGACGACGGAGTGCG | 492 |
| 46_CA_site | GGCGcaCTCCGTCGTCAGG | | CCGTCGTCGACAAGCCGGCC | |
| | ATCATCCGG | | | |
| Bxb1_AttB_ | GGCCGGCTTGTCGACGAC | 414 | CCGGATGATCCTGACGACGGAGTACG | 493 |
| 46_TA_site | GGCGtaCTCCGTCGTCAGG | | CCGTCGTCGACAAGCCGGCC | |
| | ATCATCCGG | | | |
| Bxb1_AttB_ | GGCCGGCTTGTCGACGAC | 415 | CCGGATGATCCTGACGACGGAGCTCG | 494 |
| 46_AG_site | GGCGagCTCCGTCGTCAGG | | CCGTCGTCGACAAGCCGGCC | |
| | ATCATCCGG | | | |
| Bxb1_AttB | GGCCGGCTTGTCGACGAC | 416 | CCGGATGATCCTGACGACGGAGCCCG | 495 |
| 46_GG_site | GGCGggCTCCGTCGTCAGG | | CCGTCGTCGACAAGCCGGCC | |
| | ATCATCCGG | | | |
| Bxb1_AttB_ | GGCCGGCTTGTCGACGAC | 417 | CCGGATGATCCTGACGACGGAGCGCG | 496 |
| 46_CG_site | GGCGcgCTCCGTCGTCAGG | | CCGTCGTCGACAAGCCGGCC | |
| | ATCATCCGG | | | |
| Bxb1_AttB_ | GGCCGGCTTGTCGACGAC | 418 | CCGGATGATCCTGACGACGGAGCACG | 497 |
| 46_TG_site | GGCGtgCTCCGTCGTCAGG | | CCGTCGTCGACAAGCCGGCC | |
| | ATCATCCGG | | | |
| Bxb1_AttB_ | GGCCGGCTTGTCGACGAC | 419 | CCGGATGATCCTGACGACGGAGGTCG | 498 |
| 46_AC_site | GGCGacCTCCGTCGTCAGG | | CCGTCGTCGACAAGCCGGCC | |
| | ATCATCCGG | | | |
| Bxb1_AttB_ | GGCCGGCTTGTCGACGAC | 420 | CCGGATGATCCTGACGACGGAGGCCG | 499 |
| 46_GC_site | GGCGgcCTCCGTCGTCAGG | | CCGTCGTCGACAAGCCGGCC | |
| | ATCATCCGG | | | |
| Bxb1_AttB_ | GGCCGGCTTGTCGACGAC | 421 | CCGGATGATCCTGACGACGGAGGGC | 500 |
| 46_CC_site | GGCGccCTCCGTCGTCAGG | | GCCGTCGTCGACAAGCCGGCC | |
| | ATCATCCGG | | | |
| Bxb1_AttB_ | GGCCGGCTTGTCGACGAC | 422 | CCGGATGATCCTGACGACGGAGGAC | 501 |
| 46_TC_site | GGCGtcCTCCGTCGTCAGG | | GCCGTCGTCGACAAGCCGGCC | |
| | ATCATCCGG | | | |
| Bxb1_AttB_ | GGCCGGCTTGTCGACGAC | 423 | CCGGATGATCCTGACGACGGAGATCG | 502 |
| 46_AT_site | GGCGatCTCCGTCGTCAGG | | CCGTCGTCGACAAGCCGGCC | |
| | ATCATCCGG | | | |
| Bxb1_AttB_ | GGCCGGCTTGTCGACGAC | 424 | CCGGATGATCCTGACGACGGAGAGC | 503 |
| 46_CT_site | GGCGctCTCCGTCGTCAGG | | GCCGTCGTCGACAAGCCGGCC | |
| | ATCATCCGG | | | |
| Bxb1_AttB_ | GGCCGGCTTGTCGACGAC | 425 | CCGGATGATCCTGACGACGGAGAAC | 504 |
| 46_TT_site | GGCGttCTCCGTCGTCAGG | | GCCGTCGTCGACAAGCCGGCC | |
| | ATCATCCGG | | | |
| Bxb1_AttB_ | GGCTTGTCGACGACGGCG | 426 | ATGATCCTGACGACGGAGACCGCCGT | 505 |
| 38_GT_site | GTCTCCGTCGTCAGGATC | | CGTCGACAAGCC | |
| | AT | | | |
| Bxb1_AttB_ | GGCTTGTCGACGACGGCG | 427 | ATGATCCTGACGACGGAGTTCGCCGT | 506 |
| 38_AA_site | aaCTCCGTCGTCAGGATCA | | CGTCGACAAGCC | |
| | T | | | |
| Bxb1_AttB_ | GGCTTGTCGACGACGGCG | 428 | ATGATCCTGACGACGGAGTCCGCCGT | 507 |
| 38_GA_site | gaCTCCGTCGTCAGGATCA | | CGTCGACAAGCC | |
| | T | | | |
| Bxb1_AttB_ | GGCTTGTCGACGACGGCG | 429 | ATGATCCTGACGACGGAGTGCGCCGT | 508 |
| 38_CA_site | caCTCCGTCGTCAGGATCA | | CGTCGACAAGCC | |
| | T | | | |
| Bxb1_AttB_ | GGCTTGTCGACGACGGCG | 430 | ATGATCCTGACGACGGAGTACGCCGT | 509 |
| 38_TA_site | taCTCCGTCGTCAGGATCA | | CGTCGACAAGCC | |
| | T | | | |
| Bxb1_AttB_ | GGCTTGTCGACGACGGCG | 431 | ATGATCCTGACGACGGAGCTCGCCGT | 510 |
| 38_AG_site | agCTCCGTCGTCAGGATCA | | CGTCGACAAGCC | |
| | T | | | |
| Bxb1_AttB_ | GGCTTGTCGACGACGGCG | 432 | ATGATCCTGACGACGGAGCCCGCCGT | 511 |
| 38_GG_site | ggCTCCGTCGTCAGGATCA | | CGTCGACAAGCC | |
| | T | | | |
| Bxb1_AttB_ | GGCTTGTCGACGACGGCG | 433 | ATGATCCTGACGACGGAGCGCGCCGT | 512 |
| 38_CG_site | cgCTCCGTCGTCAGGATCA | | CGTCGACAAGCC | |
| | T | | | |
| Bxb1_AttB_ | GGCTTGTCGACGACGGCG | 434 | ATGATCCTGACGACGGAGCACGCCGT | 513 |
| 38_TG_site | tgCTCCGTCGTCAGGATCA | | CGTCGACAAGCC | |
| | T | | | |
| Bxb1_AttB_ | GGCTTGTCGACGACGGCG | 435 | ATGATCCTGACGACGGAGGTCGCCGT | 514 |
| 38_AC_site | acCTCCGTCGTCAGGATCA | | CGTCGACAAGCC | |
| | T | | | |
| Bxb1_AttB_ | GGCTTGTCGACGACGGCG | 436 | ATGATCCTGACGACGGAGGCCGCCGT | 515 |
| 38_GC_site | gcCTCCGTCGTCAGGATCA | | CGTCGACAAGCC | |
| | T | | | |
| Bxb1_AttB_ | GGCTTGTCGACGACGGCG | 437 | ATGATCCTGACGACGGAGGGCGCCGT | 516 |
| 38_CC_site | ccCTCCGTCGTCAGGATCA | | CGTCGACAAGCC | |
| | T | | | |
| Bxb1_AttB_ | GGCTTGTCGACGACGGCG | 438 | ATGATCCTGACGACGGAGGACGCCGT | 517 |
| 38_TC_site | tcCTCCGTCGTCAGGATCA | | CGTCGACAAGCC | |
| | T | | | |
| Bxb1_AttB_ | GGCTTGTCGACGACGGCG | 439 | ATGATCCTGACGACGGAGATCGCCGT | 518 |
| 38_AT_site | atCTCCGTCGTCAGGATCA | | CGTCGACAAGCC | |
| | T | | | |
| Bxb1_AttB_ | GGCTTGTCGACGACGGCG | 440 | ATGATCCTGACGACGGAGAGCGCCGT | 519 |
| 38_CT_site | ctCTCCGTCGTCAGGATCA | | CGTCGACAAGCC | |
| | T | | | |
| Bxb1_AttB_ | GGCTTGTCGACGACGGCG | 441 | ATGATCCTGACGACGGAGAACGCCGT | 520 |
| 38_TT_site | ttCTCCGTCGTCAGGATCA | | CGTCGACAAGCC | |
| | T | | | |
| Cre Lox 66 | TACCGTTCGTATAATGTA | 442 | ATAACTTCGTATAGCATACATTATAC | 521 |
| site | TGCTATACGAAGTTAT | | GAACGGTA | |
| Cre Lox 71 | ATAACTTCGTATAATGTA | 443 | TACCGTTCGTATAGCATACATTATAC | 522 |
| site | TGCTATACGAACGGTA | | GAAGTTAT | |
| TP901-1 | TTTACCTTGATTGAGATGT | 444 | CACAATTAACATCTCAATCAAGGTAA | 523 |
| minimal | TAATTGTG | | A | |
| AttB site | | | | |
| TP901-1 | GCGAGTTTTTATTTCGTTT | 445 | AAAGGAGTTTTTTAGTTACCTTAATT | 524 |
| minimal | ATTTCAATTAAGGTAACT | | GAAATAAACGAAATAAAAACTCGC | |
| AttP site | AAAAAACTCCTTT | | | |
| PhiBT1 | CTGGATCATCTGGATCAC | 446 | CAGGTTTTTGACGAAAGTGATCCAGA | 525 |
| minimal | TTTCGTCAAAAACCTG | | TGATCCAG | |
| AttB site | | | | |
| PhiBT1 | TTCGGGTGCTGGGTTGTT | 447 | TGGTGCTGAGTAGTTTCCCATGGATC | 526 |
| minimal | GTCTCTGGACAGTGATCC | | ACTGTCCAGAGACAACAACCCAGCAC | |
| AttP site | ATGGGAAACTACTCAGCA | | CCGAA | |
| | CCA | | | |
| Bacillus_ | gatatggggaagtgaatc | 448 | ggtactgtggcggttgtactgattca | 527 |
| cereus_ | agtacaaccgccacagta | | cttccccatatc | |
| AH187_Int30_ | cc | | | |
| 38 bp_Att | | | | |
| Staphylococcus_ | tgggtggtacaggtgcca | 449 | cataaatggtacaactaatgtggcac | 528 |
| lugdunensis_ | cattagttgtaccattta | | ctgtaccaccca | |
| N920143_ | tg | | | |
| Int12_ | | | | |
| 38 bp_Att | | | | |
| Bacillus_ | gttgtttttccagatcca | 450 | cttatatttacaggaccaactggatc | 529 |
| cytotoxicus_ | gttggtcctgtaaatata | | tggaaaaacaac | |
| NVH_391-98_ | ag | | | |
| Int13_ | | | | |
| 38 bp_Att | | | | |
| Bacillus_ | tggggaagtgaatcagta | 451 | ctgtggcggttgtactgattcacttc | 454 |
| cereus_ | caaccgccacag | | ccca | |
| AH187_Int30_ | | | | |
| Att_30 | | | | |
| Bacillus_ | ggggaagtgaatcagtac | 452 | tgtggcggttgtactgattcacttcc | 455 |
| cereus_ | aaccgccaca | | cc | |
| AH187_Int30_ | | | | |
| Att_28 | | | | |
| Bacillus_ | gggaagtgaatcagtaca | 453 | gtggcggttgtactgattcacttccc | 456 |
| cereus_ | accgccac | | | |
| AH187_Int30_ | | | | |
| Att_26 | | | | |
| Bacillus_ | ctgtggcggttgtactga | 454 | tggggaagtgaatcagtacaaccgcc | 451 |
| cereus_ | ttcacttcccca | | acag | |
| AH187_Int30_ | | | | |
| Att_rc_30 | | | | |
| Bacillus_ | tgtggcggttgtactgat | 455 | ggggaagtgaatcagtacaaccgcca | 452 |
| cereus_AH187_ | tcacttcccc | | ca | |
| Int30_Att_rc_ | | | | |
| 28 | | | | |
| Bacillus_ | gtggcggttgtactgatt | 456 | gggaagtgaatcagtacaaccgccac | 453 |
| cereus_AH187_ | cacttccc | | | |
| Int30_Att_rc_ | | | | |
| 26 | | | | |
| Bacillus_ | tttttccagatccagttg | 457 | tatttacaggaccaactggatctgga | 460 |
| cytotoxicus_ | gtcctgtaaata | | aaaa | |
| NVH_391-98_ | | | | |
| Int13_Att_30 | | | | |
| Bacillus_ | ttttccagatccagttgg | 458 | atttacaggaccaactggatctggaa | 461 |
| cytotoxicus_ | tcctgtaaat | | aa | |
| NVH_391-98_ | | | | |
| Int13_Att_28 | | | | |
| Bacillus_ | tttccagatccagttggt | 459 | tttacaggaccaactggatctggaaa | 462 |
| cytotoxicus_ | cctgtaaa | | | |
| NVH_391-98_ | | | | |
| Int13_Att_26 | | | | |
| Bacillus_ | tatttacaggaccaactg | 460 | tttttccagatccagttggtcctgta | 457 |
| cytotoxicus_ | gatctggaaaaa | | aata | |
| NVH_391-98_ | | | | |
| Int13_Att_ | | | | |
| rc_30 | | | | |
| Bacillus_ | atttacaggaccaactgg | 461 | ttttccagatccagttggtcctgtaa | 458 |
| cytotoxicus_ | atctggaaaa | | at | |
| NVH_391-98_ | | | | |
| Int13_Att_ | | | | |
| rc_28 | | | | |
| Bacillus_ | tttacaggaccaactgga | 462 | tttccagatccagttggtcctgtaaa | 459 |
| cytotoxicus_ | tctggaaa | | | |
| NVH_391-98_ | | | | |
| Int13_Att_ | | | | |
| rc_26 | | | | |
| N680429_ | CATTATATGTTTTTACAAT | 463 | cattatatgttcttacagtatggcgg | 530 |
| 560_31_50 bp | CCGGGCCGCCATACTGTA | | cccggattgtaaaaacatataatg | |
| | AGAACATATAATG | | | |
| N191607_ | CGTTATAGGGTATTGCAG | 464 | cgttatagggtattacagtatggcgg | 531 |
| 8_101_50 bp | TACCGACCGCCATACTGT | | tcggtactgcaataccctataacg | |
| | AATACCCTATAACG | | | |
| N674992_1_ | TGTATCATTTTCATATAGT | 465 | tgtatcattttcatatagttagcacc | 532 |
| 1308_50 bp | GTGCAGGTGCTAACTATA | | tgcacactatatgaaaatgataca | |
| | TGAAAATGATACA | | | |
| N684613_54_ | TGTCTACTATGTCTTTATG | 466 | tgtctactatctgtatatgcgacaca | 533 |
| 96_50 bp | CCACATGTGTCGCATATA | | tgtggcataaagacatagtagaca | |
| | CAGATAGTAGACA | | | |
| N252616_121_ | AATGAGGTCAGACGCATG | 467 | catcgaccctgacgcatgcggaggcg | 534 |
| 74_50 bp | GAGCGCCGCCTCCGCATG | | gcgctccatgcgtctgacctcatt | |
| | CGTCAGGGTCGATG | | | |
| N683040_222_ | GTTAGTACCCAAATGATA | 468 | gttagtacccaaatgacaaaaggtca | 535 |
| 19_50 bp | AAAGGATGACCTTTTGTC | | tccttttatcatttgggtactaac | |
| | ATTTGGGTACTAAC | | | |
| N687537_173_ | GTTTATAAAACCGATGCC | 469 | cttattaaaacccgttccgcttctgt | 536 |
| 59_50 bp | GCTTTGACAGAAGCGGAA | | caaagcggcatcggttttataaac | |
| | CGGGTTTTAATAAG | | | |
| N183629_47_ | GGCCGCGAGGTCGTGTTC | 470 | ggcgtgatggtcgtgaacctcaacat | 537 |
| 40_50 bp | GTCGTCATGTTGAGGTTC | | gacgacgaacacgacctcgcggcc | |
| | ACGACCATCACGCC | | | |
| N191533_224_ | TATAAACTGATATAATTC | 471 | tctacatcttgaatatatcaagttat | 538 |
| 76_50 bp | AAAGTTATAACTTGATAT | | aactttgaattatatcagtttata | |
| | ATTCAAGATGTAGA | | | |
| N682356_188_ | TATTATATCTAAAAGCAG | 472 | aattatatctaaaagcactaagctcc | 539 |
| 20_50 bp | TATGGCGGAGCTTAGTGC | | gccatactgcttttagatataata | |
| | TTTTAGATATAATT | | | |

### 6.9. Co-Delivery of Gene Editor and Donor DNA Template

This disclosure features methods of delivering (e.g., co-delivery or dual delivery) a system capable of site-specifically integrating a template polynucleotide into the genome of a cell, where the methods includes delivering to a (i) gene editor construct and a (ii) template polynucleotide, and (iii) at least a first attachment site-containing guide (atgRNA).

This disclosure also features a method for delivering a system capable of site-specifically integrating a template polynucleotide into the genome of a cell, where the method includes: delivering a lipid nanoparticle (LNP) comprising a gene editor polynucleotide (e.g., a gene editor polynucleotide construct) and a vector comprising a template polynucleotide and at least a first attachment site-containing guide RNA (atgRNA). In some embodiments, the first atgRNA comprises (i) a domain that is capable of guiding the prime editor system to a target sequence; and (ii) a reverse transcriptase (RT) template that comprises at least a portion of a first integration recognition site. In some embodiments, where the vector comprises a polynucleotide encoding a first atgRNA, the RT template comprises the entirety of the first integration recognition site. In some embodiments, where the vector comprises a polynucleotide encoding a first atgRNA, the vector also includes a sequence encoding a nicking guide RNA (ngRNA).

This disclosure also features a method for delivering a system capable of site-specifically integrating a template polynucleotide into the genome of a cell, where the method includes: delivering a lipid nanoparticle (LNP) comprising a gene editor polynucleotide (e.g., a gene editor polynucleotide construct) and a vector comprising a template polynucleotide and a first attachment site-containing guide RNA (atgRNA) and a second attachment site-containing guide RNA (atgRNA). In some embodiments, the first atgRNA and the second atgRNA are an at least first pair of atgRNAs, wherein the at least first pair of atgRNAs have domains that are capable of guiding the prime editor system to a target sequence, the first atgRNA further includes a first RT template that comprises at least a portion of the at least first integration recognition site; and the second atgRNA further includes a second RT template that comprises at least a portion of the first integration recognition site, and the first atgRNA and the second atgRNAs collectively encode the entirety of the first integration recognition site. In such embodiments, the first atgRNA and second atgRNA include at least a 6 bp overlap (e.g., 6 bp of complementarity).

This disclosure also features a method for delivering a system capable of site-specifically integrating a template polynucleotide into the genome of a cell, where the method includes: delivering into a cell a lipid nanoparticle (LNP) comprising: (i) a gene editor polynucleotide (e.g., a gene editor polynucleotide construct), and (ii) a first attachment site-containing guide RNA (atgRNA); and a vector comprising: (i) a template polynucleotide, and (ii) a second atgRNA. In some embodiments, the first atgRNA and the second atgRNA are an at least first pair of atgRNAs, wherein the at least first pair of atgRNAs have domains that are capable of guiding the prime editor system to a target sequence, the first atgRNA further includes a first RT template that comprises at least a portion of the a first integration recognition site; and the second atgRNA further includes a second RT template that comprises at least a portion of the first integration recognition site, and the first atgRNA and the second atgRNAs collectively encode the entirety of the first integration recognition site. In such embodiments, the first atgRNA and second atgRNA include at least a 6 bp overlap (e.g., 6 bp of complementarity).

This disclosure also features a method for delivering a system capable of site-specifically integrating a template polynucleotide into the genome of a cell, where the method includes delivering: a lipid nanoparticle (LNP) comprising: (i) a gene editor polynucleotide (e.g., a gene editor polynucleotide construct), (ii) a first attachment site-containing guide RNA (atgRNA), and (iii) a second atgRNA; and a vector comprising (i) a template polynucleotide. In some embodiments, the first atgRNA and the second atgRNA are an at least first pair of atgRNAs, wherein the at least first pair of atgRNAs have domains that are capable of guiding the prime editor system to a target sequence, the first atgRNA further includes a first RT template that comprises at least a portion of the at least first integration recognition site; and the second atgRNA further includes a second RT template that comprises at least a portion of the at least first integration recognition site, and the first atgRNA and the second atgRNAs collectively encode the entirety of the first integration recognition site. In such embodiments, the first atgRNA and second atgRNA include at least a 6 bp overlap (e.g., 6 bp of complementarity).

This disclosure also features a method for delivering a system capable of site-specifically integrating a template polynucleotide into the genome of a cell, where the method includes delivering: a lipid nanoparticle (LNP) comprising: (i) a gene editor polynucleotide (e.g., a gene editor polynucleotide construct) and (ii) a first attachment site-containing guide RNA (atgRNA); and a vector comprising: (i) a template polynucleotide, and (ii) a nicking atgRNA. In some embodiments, the first atgRNA comprises (i) a domain that is capable of guiding the prime editor system to a target sequence; and (ii) a reverse transcriptase (RT) template that comprises at least a portion of a first integration recognition site. In some embodiments, where the vector comprises a polynucleotide encoding a first atgRNA, the RT template comprises the entirety of the first integration recognition site.

In some embodiments, where the method includes delivering an LNP and a first vector, the LNP and the first vector are delivered at least 1 day, 2 days, 3 days, 4 days, 5 days, 6 days, 7 days, 2 weeks, 3 weeks, 4 weeks, 5 weeks, 6 weeks, 7 weeks, or 8 weeks apart. In some embodiments, where the method includes delivering an LNP and a second vector, the LNP and the second vector are delivered at least 1 day, 2 days, 3 days, 4 days, 5 days, 6 days, 7 days, 2 weeks, 3 weeks, 4 weeks, 5 weeks, 6 weeks, 7 weeks, or 8 weeks apart.

This disclosure also features a system capable of site-specifically integrating a template polynucleotide into the genome of a cell, the system comprising: a lipid nanoparticle (LNP) comprising a gene editor polynucleotide (e.g., a gene editor polynucleotide construct) and a vector comprising a template polynucleotide and at least a first attachment site-containing guide RNA (atgRNA). In some embodiments, the first atgRNA comprises (i) a domain that is capable of guiding the prime editor system to a target sequence; and (ii) a reverse transcriptase (RT) template that comprises at least a portion of a first integration recognition site. In some embodiments, where the vector comprises a polynucleotide encoding a first atgRNA, the RT template comprises the entirety of the first integration recognition site. In some embodiments, where the vector comprises a polynucleotide encoding a first atgRNA, the vector also includes a sequence encoding a nicking guide RNA (ngRNA).

This disclosure also features a system capable of site-specifically integrating a template polynucleotide into the genome of a cell, the system comprising: a lipid nanoparticle (LNP) comprising a gene editor polynucleotide (e.g., a gene editor polynucleotide construct) and a vector comprising a template polynucleotide and a first attachment site-containing guide RNA (atgRNA) and a second attachment site-containing guide RNA (atgRNA). In some embodiments, the first atgRNA and the second atgRNA are an at least first pair of atgRNAs, wherein the at least first pair of atgRNAs have domains that are capable of guiding the prime editor system to a target sequence, the first atgRNA further includes a first RT template that comprises at least a portion of the a first integration recognition site; and the second atgRNA further includes a second RT template that comprises at least a portion of the first integration recognition site, and the first atgRNA and the second atgRNAs collectively encode the entirety of the first integration recognition site. In such embodiments, the first atgRNA and second atgRNA include at least a 6 bp overlap.

This disclosure also features a system capable of site-specifically integrating a template polynucleotide into the genome of a cell, the system comprising: a lipid nanoparticle (LNP) comprising: (i) a gene editor polynucleotide (e.g., a gene editor polynucleotide construct), and (ii) a first attachment site-containing guide RNA (atgRNA); and a vector comprising: (i) a template polynucleotide, and (ii) a second atgRNA. In some embodiments, the first atgRNA and the second atgRNA are an at least first pair of atgRNAs, wherein the at least first pair of atgRNAs have domains that are capable of guiding the prime editor system to a target sequence, the first atgRNA further includes a first RT template that comprises at least a portion of the a first integration recognition site; and the second atgRNA further includes a second RT template that comprises at least a portion of the first integration recognition site, and the first atgRNA and the second atgRNAs collectively encode the entirety of the first integration recognition site. In such embodiments, the first atgRNA and second atgRNA include at least a 6 bp overlap.

This disclosure also features a system capable of site-specifically integrating a template polynucleotide into the genome of a cell, the system comprising: co-delivering: a lipid nanoparticle (LNP) comprising: (i) a gene editor polynucleotide (e.g., a gene editor polynucleotide construct), (ii) a first attachment site-containing guide RNA (atgRNA), and (iii) a second atgRNA; and a vector comprising (i) a template polynucleotide. In some embodiments, the first atgRNA and the second atgRNA are an at least first pair of atgRNAs, wherein the at least first pair of atgRNAs have domains that are capable of guiding the prime editor system to a target sequence, the first atgRNA further includes a first RT template that comprises at least a portion of the a first integration recognition site; and the second atgRNA further includes a second RT template that comprises at least a portion of the first integration recognition site, and the first atgRNA and the second atgRNAs collectively encode the entirety of the first integration recognition site. In such embodiments, the first atgRNA and second atgRNA include at least a 6 bp overlap.

This disclosure also features a system capable of site-specifically integrating a template polynucleotide into the genome of a cell, the system comprising: a lipid nanoparticle (LNP) comprising: (i) a gene editor polynucleotide (e.g., a gene editor polynucleotide construct) and (ii) a first attachment site-containing guide RNA (atgRNA); and a vector comprising: (i) a template polynucleotide, and (ii) a nicking atgRNA. In some embodiments, the first atgRNA comprises (i) a domain that is capable of guiding the prime editor system to a target sequence; and (ii) a reverse transcriptase (RT) template that comprises at least a portion of a first integration recognition site. In some embodiments, where the vector comprises a polynucleotide encoding a first atgRNA, the RT template comprises the entirety of the first integration recognition site.

In typical embodiments, the LNP comprising a gene editor polynucleotide construct is capable delivering to a cell cytoplasm the gene editor polynucleotide construct. In some embodiments, the LNP comprising a gene editor polynucleotide construct is capable delivering to a cell nucleus the gene editor polynucleotide construct. In some embodiments, the LNP comprises a gene editor protein and associated guide nucleic acids. In some embodiments, the LNP comprises a gene editor protein and associated guide nucleic acids that are capable of localizing to cell nucleus.

In some embodiments, a gene editor polynucleotide construct is delivered to a cell by a fusosome. In some embodiments, a gene editor polynucleotide construct is delivered to a cell cytoplasm by a fusosome. In some embodiments, the fusosome comprises a gene editor protein and associated guide nucleic acids.

In some embodiments, a gene editor polynucleotide construct is delivered to a cell by an exosome. In some embodiments, a gene editor polynucleotide construct is delivered to a cell cytoplasm by an exosome. In some embodiments, the exosome comprises a gene editor protein and associated guide nucleic acids.

In some embodiments, the prime editor or Gene Writer protein fusion, either of which may have a fused/linked integrase, is incorporated (i.e., packaged) into LNP as protein. Further, associated atgRNA and optional ngRNAs may be co-packaged with gene editor proteins in LNP.

In some embodiments, the gene editor polynucleotide construct comprises (a) a polynucleotide sequence encoding a prime editor fusion protein or a Gene Writer™ protein, (b) a polynucleotide sequence encoding an attachment site-containing guide RNA (atgRNA), (c) optionally, a polynucleotide sequence encoding a nickase guide RNA (ngRNA), (d) a polynucleotide sequence encoding an integrase, (e) and optionally, a polynucleotide sequence encoding a recombinase.

In some embodiments, the prime editor or Gene Writer protein fusion, either of which may have a fused/linked integrase, is expressed as a split construct. In typical embodiments, the split construct in reconstituted in a cell. In some embodiments, the split construct can be fused or ligated via intein protein splicing. In some embodiments, the split construct can be reconstituted via protein-protein inter-molecular bonding and/or interactions. In some embodiments, the split construct can be reconstituted via chemical, biological, or environmental induced oligomerization. In certain embodiments, the split construct can be adapted into one or more nucleic acid constructs described herein.

### 6.9.1. Gene Editor Polynucleotide

In some embodiments, the systems described include a gene editor polynucleotide that is delivered to a cell using the methods described herein. In some embodiments, the gene editor polynucleotide is delivered as a polynucleotide (e.g., an mRNA). In some embodiments, the gene editor polynucleotide is delivered as a protein. In some embodiments, the gene editor polynucleotide or protein is packaged, and thereby vectorized, within a lipid nanoparticle (LNP). In some embodiments, the gene editor polynucleotide or protein is packaged in a LNP and is co-delivered with a template polynucleotide (i.e., nucleic acid “cargo” or nucleic acid “payload”) packaged into a separate vector (e.g., a viral vector (e.g., an AAV or adenovirus)) or a second lipid nanoparticle (LNP).

In some embodiments, the gene editor polynucleotide is delivered to the cells as a polynucleotide. For example, the gene editor polynucleotide is delivered to the cells as an mRNA encoding the gene editor polynucleotide (e.g., the gene editor protein or the prime editor system). In some embodiments, the mRNA comprises one or more modified uridines. In some embodiments, the mRNA comprises a sequence where each of the uridines is a modified uridine. In some embodiments, the mRNA is uridine depleted. In some embodiments, the mRNA encoding the nickase comprises one or more modified uridines. In some embodiments, the mRNA encoding the reverse transcriptase comprises one or more modified uridines. In some embodiments, the mRNA encoding the nickase comprises one or more modified uridines, and the mRNA encoding the reverse transcriptase comprises one or more modified uridines. In some embodiments, where the integrase is encoded in an mRNA, the mRNA comprises modified uridines. In some embodiments, a modified uridine is a N1-Methylpseudouridine-5′-Triphosphate. In some embodiments, a modified uridine is a pseudouridine. In some embodiments, the mRNA comprises a 5′ cap. In some embodiments, the 5′ cap comprises a molecular formula of C₃₂H₄₃N₁₅O₂₄P₄(free acid).

In some embodiments, the gene editor polynucleotide (e.g., a gene editor polynucleotide construct) comprises a polynucleotide sequence encoding a primer editor system (e.g., any of the prime editor systems described herein). In some embodiments, the prime editor system comprises a nucleotide sequence encoding a nickase (e.g., any of the Cas proteins or variants thereof (e.g., nickases) and nickases described herein, see Tables 4-8) and a nucleotide sequence encoding a reverse transcriptase (e.g., any of the reverse transcriptases described herein). In some embodiments, the nucleotide sequence encoding the nickase and the nucleotide sequence encoding the reverse transcriptase are positioned in the construct such that when expressed the nickase is linked to the reverse transcriptase. In some embodiments, the nickase is linked to the reverse transcriptase by in-frame fusion. In some embodiments, the nickase is linked to the reverse transcriptase by a linker. In some embodiments, the linker is a peptide fused in-frame between the nickase and reverse transcriptase.

In some embodiments, the gene editor polynucleotide (e.g., a gene editor polynucleotide construct) further comprises a polynucleotide sequence encoding at least a first integrase (e.g., any of the integrases described herein, e.g., as described in Table 10 and also in Yarnall et al., Nat. Biotechnol., 2022, doi.org/10.1038/s41587-022-01527-4 and Durrant et al., Nat. Biotechnol., 2022, doi.org/10.1038/s41587-022-01494-w, each of which are herein incorporated by reference in their entireties). In some embodiments, the linked nickase-reverse transcriptase are further linked to the first integrase.

In some embodiments, the gene editor polynucleotide construct further comprises a polynucleotide sequence encoding at least a first recombinase (e.g., any of the recombinases described herein).

### 6.9.2. Vector

In some embodiments, the systems and methods described herein include a vector that is capable of co-delivering a template polynucleotide, one or more attachment site-containing gRNA, one or more integrases, one or more recombinases, a gene editor polynucleotide, one or more integration recognition sites, one or more recombinase recognition sites, or a combination thereof.

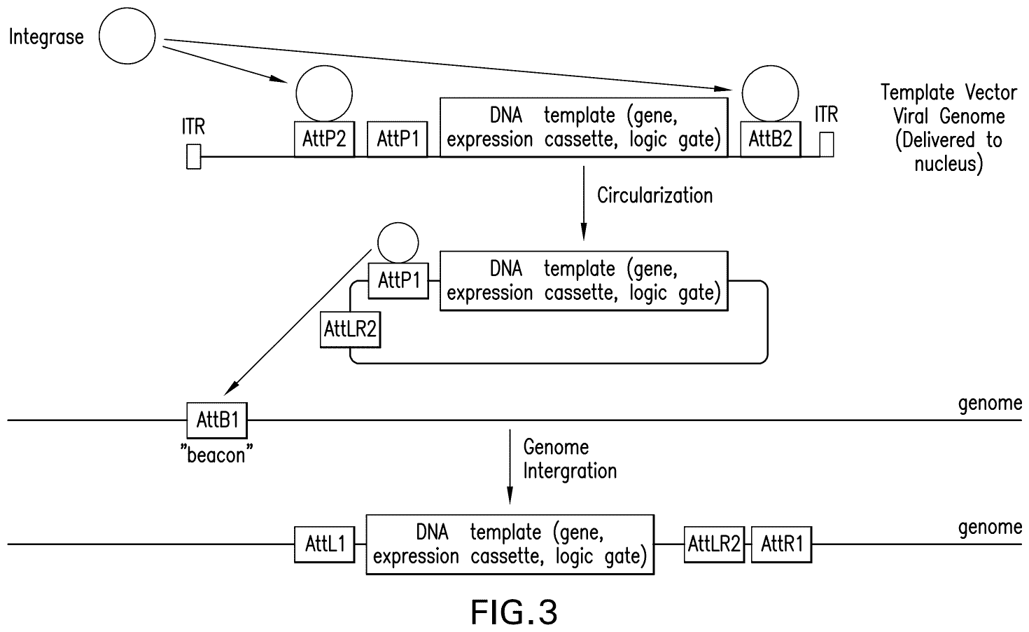

Non-limiting examples of vectors that can be used in the methods or systems described herein include the vectors described in FIGS. 3-6.

6.9.2.1 AtgRNA and/or ngRNA

In some embodiments, the vector includes a polynucleotide sequence encoding an attachment site-containing guide RNA (atgRNA). In such embodiments, the polynucleotide sequence encoding the attachment site-containing guide RNA (atgRNA) is operably linked to a regulatory element (e.g., a U6 promoter) that is capable of driving expression of the atgRNA. In such embodiments, the atgRNA comprises (i) a domain that is capable of guiding the prime editor system to a target sequence; and (ii) a reverse transcriptase (RT) template that comprises at least a portion of a first integration recognition site. In some embodiments, where the system, and thereby the vector, include a polynucleotide encoding only a first atgRNA, the RT template comprises the entirety of the first integration recognition site. In such embodiments, the vector or the LNP includes a polynucleotide sequence encoding a nicking gRNA.

In some embodiments, the vector includes a polynucleotide sequence encoding a first attachment site-containing guide RNA (atgRNA) and a polynucleotide sequence encoding a second attachment site-containing guide RNA (atgRNA). In such embodiments, the first atgRNA and the second atgRNA are an at least first pair of atgRNAs, wherein the at least first pair of atgRNAs have domains that are capable of guiding the prime editor system to a target sequence, the first atgRNA further includes a first RT template that comprises at least a portion of the a first integration recognition site; the second atgRNA further includes a second RT template that comprises at least a portion of the first integration recognition site, and the first atgRNA and the second atgRNAs collectively encode the entirety of the first integration recognition site. In such embodiments, the first atgRNA and second atgRNA include at least a 6 bp overlap.

### 6.9.2.2 Template Polynucleotide

In typical embodiments, the vector includes a template polynucleotide and a sequence that is an integration cognate of an integration recognition site site-specifically incorporated into the genome of a cell. For example, the vector includes a template polynucleotide and a second integration recognition site that is a cognate pair with the first integration recognition site site-specifically incorporated into the genome of the cell. In such embodiments, the sequence that is an integration cognate (e.g., a second integration recognition site) enables integration of the template polynucleotide or portion thereof when contacted with an integrase and the site-specifically incorporated first integration recognition site.

In typical embodiments, the vector comprising a template polynucleotide is a recombinant adenovirus, a helper dependent adenovirus, an AAV, a lentivirus, an HSV, an annelovirus, a retrovirus, a Doggybone™ DNA (dbDNA), a minicircle, a plasmid, a miniDNA, an exosome, a fusosome, or an nanoplasmid. In preferred embodiments, the vector is capable of localizing to the nucleus.

In certain embodiments, the template polynucleotide is delivered to the cytoplasm and localizes to the nucleus. In certain embodiments, the template polynucleotide is delivered to the cytoplasm by LNP. In certain embodiments, the donor template polynucleotide construct comprises a recognition sequence that is recognized by a DNA binding protein (DNA binding domain) or a transcription factor binding domain. In certain embodiments, the donor template polynucleotide construct is delivered to the nucleus by an integrase or recombinase.

In certain embodiments, the template polynucleotide is delivered to the mitochondria. In certain embodiments, the donor template polynucleotide construct comprises a mitochondria targeting sequence.

In certain embodiments, the vector comprising a template polynucleotide is AAV. In some embodiments, the AAV contains a 5′ inverted terminal repeat (ITR). In some embodiments, the AAV contains a 3′ inverted terminal repeat (ITR). In some embodiments, the AAV contains a 5′ and a 3′ ITR. In some embodiments, the 5′ and 3′ ITR are not derived from the same serotype of virus. In some embodiments, the ITRs are derived from adenovirus, AAV2, and/or AAV5.

In certain embodiments, the vector comprising a template polynucleotide is single stranded AAV (ssAAV). In certain embodiments, the vector comprising a donor template polynucleotide construct is self-complementary AAV (scAAV).

In some embodiments, a vector comprises an attachment site-containing guideRNA (atgRNA), a nicking-guideRNA (ngRNA), and template polynucleotide. In typical embodiments, the vector comprising an attachment site-containing guideRNA (atgRNA), a nicking-guideRNA (ngRNA), and template polynucleotide is recombinant adenovirus, helper dependent adenovirus, AAV, lentivirus, HSV, annelovirus, retrovirus, Doggybone™ DNA (dbDNA), minicircle, plasmid, miniDNA, exosome, fusosome, or nanoplasmid. In preferred embodiments, the vector is capable of localizing to the nucleus. In typical embodiments, the attachment site-containing guideRNA (atgRNA) sequence and the nicking-guideRNA (ngRNA) sequence contain a terminal poly dT.

In some embodiments, a vector comprises an attachment site-containing guideRNA (atgRNA), and donor template. In typical embodiments, the vector comprising an attachment site-containing guideRNA (atgRNA) and donor template is recombinant adenovirus, helper dependent adenovirus, AAV, lentivirus, HSV, annelovirus, retrovirus, Doggybone™ DNA (dbDNA), minicircle, plasmid, miniDNA, exosome, fusosome, or nanoplasmid. In preferred embodiments, the vector is capable of localizing to the nucleus. In typical embodiments, the attachment site-containing guideRNA (atgRNA) sequence contain a terminal poly dT.

In typical embodiments, the template polynucleotide is capable of being integrated into a genomic locus that contains an integrase target recognition site or a recombinase target recognition site.

In certain embodiments, the template polynucleotide comprises at least one of the following: a gene, a gene fragment, an expression cassette, a logic gate system, or any combination thereof. In some embodiments, the template polynucleotide comprises at least one intron or exon.

In typical embodiments, the template polynucleotide further comprises at least one integrase target recognition site or a recombinase target integrase site. In certain embodiments, at least one integrase target recognition site or a recombinase target integrase site is placed within the donor template vector inverted terminal repeat.

### 6.9.2.3 Integrase- or Recombinase-Mediated Self-Circularization of a Subsequence of a Vector Delivered as Part of the Co-Delivery System

In some embodiments, the delivery system (e.g., co-delivery system) includes a vector having a sub-sequence that is capable of self-circularizing to form a self-circular nucleic acid. In some embodiments, the vector comprises a physical portion or region of the vector that is capable of self-circularizing to form a circular construct. As used herein, the term “sub-sequence” refers to a portion of the vector that is capable of self-circularizing, where the sub-sequence is flanked by integration recognition sites or recombinase recognition sites positioned to enable self-circularization. As used herein, the term “self-circular nucleic acid” refers to a double-stranded, circular nucleic acid construct produced as a result of recombination of a cognate pair of integrase or recombinase recognition sites present on the vector. Recombination occurs when the vector is contacted with an integrase or a recombinase under conditions that allow for recombination of the cognate pair of integrase or recombinase recognition sites.

In some embodiments, the sub-sequence of the vector includes a first recombinase recognition site and a second recombinase recognition site, wherein the first and second recombinase recognition sites are capable of being recombined by a recombinase. In some embodiments, the sub-sequence of the vector includes a first recombinase recognition site, a second recombinase recognition site, and a second integration recognition site (e.g., the second integration recognition site is a cognate pair of the first integration recognition site), where the first and second recombinase recognition sites flank the integration recognition site. In such cases, the first recombinase recognition site, the second recombinase recognition, and a recombinase enable the self-circularizing and formation of the circular construct.

In some embodiments, the sub-sequence of the vector includes a third integration recognition site and a fourth integration recognition site, wherein the third and fourth integration recognition sites are a cognate pair. In some embodiments, the subsequence of the vector includes the second integration recognition site, the third integration recognition site, the fourth integration recognition site, where the third and fourth integration recognition sites flank the second integration recognition site (where the second integration recognition site is a cognate pair of the first integration recognition site). In such cases, the third integration recognition site, the fourth integration recognition site, and an integrase enable self-circularization and formation of the circular construct. In such cases, the third integration recognition site and/or the fourth integration recognition sites cannot recombine with the first integration recognition site and/or the second integration recognition site due, in part, to having different central dinucleotides than the first and second integration recognition sites.

In some embodiments where the subsequence includes three or more integration recognition sites, each integration recognition site or each pair of integration recognition is capable of being recognized by a different integrase. In some embodiments where the subsequence includes three or more integration recognition sites, each integration recognition site or each pair of integration recognition comprises a different central dinucleotide.

In some embodiments, self-circularizing is mediated at the integration recognition sites or recombinase recognition sites. In some embodiments, the self-circularizing is mediated by an integrase or a recombinase.

In some embodiments, upon introducing the vector into a cell and after self-circularizing to form the self-circular nucleic acid, the self-circular nucleic acid comprising the second integration recognition site is capable of being integrated into the cell's genome at the target sequence that contains the first integration recognition site.

In some embodiments, following self-circularization, the self-circular nucleic acid comprises one or more additional integration recognition sites that enable integration of an additional nucleic acid cargo. In such cases, the additional nucleic acid cargo includes a sequence that is a cognate pair with one or more of the additional integration recognition sites in the self-circular nucleic acid. For example, integration of the self-circular nucleic acid into the genome of a cell results in integration of the one or more additional integration recognition sites into the genome along with the nucleic acid cargo. The integrated one or more additional integration recognition sites serve as an integration recognition site (beacon) for placing the additional nucleic acid cargo. Upon contacting the cell harboring the integrated nucleic acid cargo and the one or more additional integration recognition sites with an integrase and the second additional nucleic acid cargo that includes a sequence that is an integration cognate to the one or more additional integration recognition sites the additional nucleic acid cargo is integrated into the cell's genome.

In typical embodiments, the self-circularized nucleic acid comprises a DNA cargo. embodiments, the DNA cargo is a gene or gene fragment. In some embodiments the DNA cargo is an expression cassette. In some embodiments, the DNA cargo is a logic gate or logic gate system. The logic gate or logic gate system may be DNA based, RNA based, protein based, or a mix of DNA, RNA, and protein. In some embodiments, the nucleic acid cargo is a genetic, protein, or peptide tag and/or barcode.

### 6.9.2.4 A Second Vector

In some embodiments, the system or methods described herein include a second vector. In some embodiments, where the gene editor polynucleotide encodes a prime editor system comprising a nickase (e.g., any of the Cas proteins or variants thereof (e.g., nickases) and nickases described herein, see Tables 4-8) and a reverse transcriptase (e.g., any of the reverse transcriptase described herein), the second vector comprises a polynucleotide sequence encoding an integrase (e.g., any of the integrases described herein, e.g., as described in Table 10 and also in Yarnall et al., Nat. Biotechnol., 2022, doi.org/10.1038/s41587-022-01527-4 and Durrant et al., Nat. Biotechnol., 2022, doi.org/10.1038/s41587-022-01494-w, each of which are herein incorporated by reference in their entireties).

In some embodiments, where the gene editor polynucleotide encodes a prime editor system comprising a nickase and a reverse transcriptase, the second vector comprises a polynucleotide sequence encoding at least a first recombinase. In some embodiments, where the gene editor polynucleotide encodes a prime editor system comprising a nickase, a reverse transcriptase, and an integrase, the second vector comprises a polynucleotide sequence encoding at least a first recombinase. In some embodiments, where the gene editor polynucleotide encodes a prime editor system comprising a nickase, a reverse transcriptase, and an integrase, the second vector comprises a polynucleotide sequence encoding at least a second integrase.

In some embodiments, the second vector includes a template polynucleotide and a sequence that is an integration cognate of an integration recognition site site-specifically incorporated into the genome of a cell. For example, the second vector includes a template polynucleotide and a second integration recognition site that is a cognate pair with the first integration recognition site site-specifically incorporated into the genome of the cell. In such embodiments, the sequence that is an integration cognate (e.g., a second integration recognition site) enables integration of the template polynucleotide or portion thereof when contacted with an integrase and the site-specifically incorporated first integration recognition site.

In some embodiments, the second vector is a vector selected from: adenovirus, AAV, lentivirus, HSV, annelovirus, retrovirus, Doggybone™ DNA (dbDNA), minicircle, plasmid, miniDNA, exosome, fusosome, or nanoplasmid.

In some embodiments, the polynucleotide sequence encoding the prime editor system is encoded on at least two different vectors. In one embodiment, a first vector comprises a polynucleotide sequence encoding a nickase and a second vector comprises a polynucleotide sequence encoding a reverse transcriptase. In such cases, the first vector and second are delivered concurrently.

In some embodiments, the polynucleotide sequence(s) encoding the prime editor system is encoded on at least two (non-contiguous) polynucleotide sequences. In one embodiment, a first polynucleotide sequence encodes a nickase and a second polynucleotide sequence encodes a reverse transcriptase. In such cases, the first vector and second are delivered concurrently (e.g., in a first LNP).

### 6.9.3. Split Lipid Nanoparticles (LNPs)

Also provided herein are methods of co-delivering a system capable of site-specifically integrating at least a first integration recognition site into the genome of a cell, where the method includes delivering to a cell a mixture of a first LNP and a second LNP (“split LNPs”). In one embodiment, the method includes co-delivering to a cell a first gene editor polynucleotide construct and a first attachment site-containing guide RNA (atgRNA) are packaged, and thereby vectorized, within the first LNP, and a second gene editor polynucleotide construct and a second attachment site containing guide RNR (atgRNA) are packaged, and thereby vectorized, within the second LNP, where the first atgRNA and the second atgRNA are an at least first pair of atgRNA. The at least first pair of atgRNAs comprise domains that are capable of guiding the prime editor system to a target sequence. The first atgRNA further includes a first RT template that comprises at least a portion of a first integration recognition site. The second atgRNA further includes a second RT template that comprises at least a portion of the first integration recognition site. The first atgRNA and the second atgRNAs collectively encode the entirety of the first integration recognition site. In such embodiments, the first atgRNA and second atgRNA include at least a 6 bp overlap.

In some embodiments, where the method includes delivering a first LNP (e.g., a first LNP comprising a first gene editor polynucleotide construct and a first atgRNA) and a second LNP (e.g., a second LNP comprising a second gene editor polynucleotide construct and a second atgRNA), the first LNP and the second LNP are mixed prior to delivering to a cell. In some embodiments, the first LNP and the second LNP are mixed at a ratio of first LNP to second LNP of 1:10, 1:9, 1:8, 1:7, 1:6, 1:5, 1:4, 1:3, 1:2, 1:1, 1:0.75, 0.75:1, 2:1, 3:1, 4:1, 5:1, 6:1, 7:1, 8:1, 9:1, or 10:1. In some embodiments, the first LNP and the second LNP are mixed at a ratio of 1:1.

In some embodiments, a first LNP comprising a first gene editor polynucleotide construct and a first attachment site-containing guide RNA (atgRNA1) comprises a ratio of ratio of gene editor polynucleotide construct (e.g., mRNA) to atgRNA1 of 1:10, 1:9, 1:8, 1:7, 1:6, 1:5, 1:4, 1:3, 1:2, 1:1, 1:0.75, 0.75:1, 2:1, 3:1, 4:1, 5:1, 6:1, 7:1, 8:1, 9:1, or 10:1. In some embodiments, the first LNP comprises a ratio of mRNA to atgRNA1 of 2:1.

In some embodiments, a second LNP comprising a second gene editor polynucleotide construct and a second attachment site-containing guide RNA (atgRNA2) comprises a ratio of gene editor polynucleotide construct (e.g., mRNA) to atgRNA2 of 1:10, 1:9, 1:8, 1:7, 1:6, 1:5, 1:4, 1:3, 1:2, 1:1, 1:0.75, 0.75:1, 2:1, 3:1, 4:1, 5:1, 6:1, 7:1, 8:1, 9:1, or 10:1. In some embodiments, the second LNP comprises a ratio of mRNA to atgRNA2 of 2:1.

In some embodiments, where the method includes delivering a first LNP (e.g., a first LNP comprising a first gene editor polynucleotide construct and a first atgRNA) and a second LNP (e.g., a second LNP comprising a second gene editor polynucleotide construct and a second atgRNA), the first LNP and the second LNP are mixed such that the ratio of gene editor polynucleotide construct (e.g., mRNA) to first atgRNA (atgRNA1) to second atgRNA (atgRNA2) is 1:0.25:0.25, 1:0.5:0.5, 1:0.75:0.75, or 1:1:1.

In some embodiments, the method of co-delivering to a cell a mixture of LNPs includes co-delivering three or more LNPs, four or more LNPs, five or more LNPs, six or more LNPs, seven or more LNPs, eight or more LNPs, nine or more LNPs, or ten or more LNPs.

Also provided herein is a system capable of site-specifically integrating at least a first integration recognition site into the genome of a cell, the system comprising: a first gene editor polynucleotide construct and a first attachment site-containing guide RNA (atgRNA) are packaged, and thereby vectorized, within the first LNP, and a second gene editor polynucleotide construct and a second attachment site containing guide RNR (atgRNA) are packaged, and thereby vectorized, within the second LNP, where the first atgRNA and the second atgRNA are an at least first pair of atgRNA. The at least first pair of atgRNAs comprise domains that are capable of guiding the prime editor system to a target sequence. The first atgRNA further includes a first RT template that comprises at least a portion of a first integration recognition site. The second atgRNA further includes a second RT template that comprises at least a portion of the first integration recognition site. The first atgRNA and the second atgRNAs collectively encode the entirety of the first integration recognition site. In such embodiments, the first atgRNA and second atgRNA include at least a 6 bp overlap.

In some embodiments, the system comprises a first LNP (e.g., any of the first LNPs described herein) and a second LNP (e.g., any of the second LNPs described herein) at a ratio of first LNP to second LNP of 1:10, 1:9, 1:8, 1:7, 1:6, 1:5, 1:4, 1:3, 1:2, 1:1, 1:0.75, 0.75:1, 2:1, 3:1, 4:1, 5:1, 6:1, 7:1, 8:1, 9:1, or 10:1. In some embodiments, the system comprise the first LNP and the second LNP at a ratio of 1:1.

In some embodiments, the system comprises a first LNP having a ratio of a first gene editor polynucleotide construct to a first attachment site-containing guide RNA (atgRNA1) of 1:10, 1:9, 1:8, 1:7, 1:6, 1:5, 1:4, 1:3, 1:2, 1:1, 1:0.75, 0.75:1, 2:1, 3:1, 4:1, 5:1, 6:1, 7:1, 8:1, 9:1, or 10:1. In some embodiments, the system includes a first LNP having a ratio of mRNA (i.e., mRNA encoding the gene editor protein) to atgRNA1 of 2:1.

In some embodiments, the system comprise a second LNP having a ratio of a second gene editor polynucleotide construct to a second attachment site-containing guide RNA (atgRNA2) of 1:10, 1:9, 1:8, 1:7, 1:6, 1:5, 1:4, 1:3, 1:2, 1:1, 1:0.75, 0.75:1, 2:1, 3:1, 4:1, 5:1, 6:1, 7:1, 8:1, 9:1, or 10:1. In some embodiments, the system includes a second LNP having a ratio of mRNA (i.e., mRNA encoding the gene editor protein) to atgRNA2 of 2:1.

In some embodiments, the system comprises a ratio of gene editor polynucleotide construct (e.g., mRNA encoding the gene editor protein) to first atgRNA (atgRNA1) to second atgRNA (atgRNA2) of 1:0.25:0.25, 1:0.5:0.5, 1:0.75:0.75, or 1:1:1.

In some embodiments, the system comprises a mixture of LNPs comprising three or more LNPs, four or more LNPs, five or more LNPs, six or more LNPs, seven or more LNPs, eight or more LNPs, nine or more LNPs, or ten or more LNPs.

In some embodiments, where a split LNP (e.g., a mixture of two LNPs packaged with different cargo) is being used to site-specifically integrate the at least first integration recognition site into the genome, a vector comprising a template polynucleotide and a sequence that is an integration cognate (i.e., cognate to an integration recognition site site-specifically incorporated into the genome of a cell) can be delivered to the cell concurrently with the split LNPs or after delivery of the split LNPs. For example, after delivering the split LNPs to the cell, a vector that includes a template polynucleotide and a second integration recognition site that is a cognate pair with the first integration recognition site is delivered to the cell. In such embodiments, the sequence that is an integration cognate (e.g., a second integration recognition site) enables integration of the template polynucleotide or portion thereof when contacted with an integrase and the site-specifically incorporated first integration recognition site.

### 6.9.4. Vector Delivery of a Template Polynucleotide

In certain aspects the invention involves vectors, e.g. for delivering or introducing in a cell, but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety.

Vector delivery, e.g., plasmid, viral delivery: The CRISPR enzyme, for instance a Type V protein such as C2c1 or C2c3, and/or any of the present RNAs, for instance a guide RNA, can be delivered using any suitable vector, e.g., plasmid or viral vectors, such as adeno associated virus (AAV), lentivirus, adenovirus or other viral vector types, or combinations thereof. Effector proteins and one or more guide RNAs can be packaged into one or more vectors, e.g., plasmid or viral vectors. In some embodiments, the vector, e.g., plasmid or viral vector is delivered to the tissue of interest by, for example, an intramuscular injection, while other times the delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other compounds known in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹¹ particles), more preferably at least about 1×10⁷ particles, more preferably at least about 1×10⁸ particles (e.g., about 1×10⁸-1×10¹¹ particles or about 1×10⁹-1×10¹² particles), and most preferably at least about 1×10¹⁰ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ particles). Thus, the dose may contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10⁵⁰ functional AAV/ml solution. The dosage may be adjusted to balance the therapeutic benefit against any side effects. In an embodiment herein, the AAV dose is generally in the range of concentrations of from about 1×10⁵ to 1×10⁵⁰ genomes AAV (sometimes referred to herein as “vector genomes” or “vg”), from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

The promoter used to drive nucleic acid-targeting effector protein coding nucleic acid molecule expression can include: AAV ITR can serve as a promoter: this is advantageous for eliminating the need for an additional promoter element (which can take up space in the vector). The additional space freed up can be used to drive the expression of additional elements (gRNA, etc.). Also, ITR activity is relatively weaker, so can be used to reduce potential toxicity due to over expression of nucleic acid-targeting effector protein. For ubiquitous expression, can use promoters: CMV, CAG, CBh, PGK, SV40, Ferritin heavy or light chains, etc. For brain or other CNS expression, can use promoters: SynapsinI for all neurons, CaMKIIalpha for excitatory neurons, GAD67 or GAD65 or VGAT for GABAergic neurons, etc. For liver expression, can use Albumin promoter. For lung expression, can use SP-B. For endothelial cells, can use ICAM. For hematopoietic cells can use IFNbeta or CD45. For Osteoblasts can use OG-2.

The promoter used to drive guide RNA can include: Pol III promoters such as U6 or H1 Use of Pol II promoter and intronic cassettes to express guide RNA Adeno Associated Virus (AAV).

Nucleic acid-targeting effector protein and one or more guide RNA can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses may be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific genome modification, the expression of nucleic acid-targeting effector can be driven by a cell-type specific promoter. For example, liver-specific expression might use the Albumin promoter and neuron-specific expression (e.g., for targeting CNS disorders) might use the Synapsin I promoter.

In terms of in vivo delivery, AAV is advantageous over other viral vectors for a couple of reasons: Low toxicity (this may be due to the purification method not requiring ultra centrifugation of cell particles that can activate the immune response) and Low probability of causing insertional mutagenesis because it doesn't integrate into the host genome.

AAV has a packaging limit of 4.5 or 4.75 Kb. This means that nucleic acid-targeting effector protein (such as a Type V protein such as C2c1 or C2c3) as well as a promoter and transcription terminator have to be all fit into the same viral vector. Therefore embodiments of the invention include utilizing homologs of nucleic acid-targeting effector protein (such as a Type V protein such as C2c1 or C2c3) that are shorter.

As to AAV, the AAV can be AAV1, AAV2, AAV5 or any combination thereof. One can select the AAV of the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The herein promoters and vectors are preferred individually.

Packaging cells are typically used to form virus particles that are capable of infecting a host cell. Such cells include 293 cells, which package adenovirus, and psi2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

Millington-Ward et al. (Molecular Therapy, vol. 19 no. 4, 642-649 April 2011) describes adeno-associated virus (AAV) vectors to deliver an RNA interference (RNAi)-based rhodopsin suppressor and a codon-modified rhodopsin replacement gene resistant to suppression due to nucleotide alterations at degenerate positions over the RNAi target site. An injection of either 6.0×10⁸ vp or 1.8×10¹⁰ vp AAV were subretinally injected into the eyes by Millington-Ward et al. The AAV vectors of Millington-Ward et al. may be applied to the system of the present invention, contemplating a dose of about 2×10¹¹ to about 6×10¹¹ vp administered to a human.

Dalkara et al. (Sci Transl Med 5, 189ra76 (2013)) also relates to in vivo directed evolution to fashion an AAV vector that delivers wild-type versions of defective genes throughout the retina after noninjurious injection into the eyes' vitreous humor. Dalkara describes a 7 mer peptide display library and an AAV library constructed by DNA shuffling of cap genes from AAV1, 2, 4, 5, 6, 8, and 9. The rcAAV libraries and rAAV vectors expressing GFP under a CAG or Rho promoter were packaged and deoxyribonuclease-resistant genomic titers were obtained through quantitative PCR. The libraries were pooled, and two rounds of evolution were performed, each consisting of initial library diversification followed by three in vivo selection steps. In each such step, P30 rho-GFP mice were intravitreally injected with 2 ml of iodixanol-purified, phosphate-buffered saline (PBS)-dialyzed library with a genomic titer of about 1.times.10.sup.12 vg/ml. The AAV vectors of Dalkara et al. may be applied to the nucleic acid-targeting system of the present invention, contemplating a dose of about 1×10¹⁵ to about 1×10¹⁶ vg/ml administered to a human.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SW), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and yr2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a host cell is transiently or non-transiently transfected with one or more vectors described herein. In some embodiments, a cell is transfected as it naturally occurs in a subject. In some embodiments, a cell that is transfected is taken from a subject. Cells taken from a subject include, but are not limited to, hepatocytes or cells isolated from muscle, the CNS, eye or lung. Immunological cells are also contemplated, such as but not limited to T cells, HSCs, B-cells and NK cells.

Another useful method to deliver proteins, enzymes, and guides comprises transfection of messenger RNA (mRNA). Examples of mRNA delivery methods and compositions that may be utilized in the present disclosure including, for example, PCT/US2014/028330, U.S. Pat. No. 8,822,663B2, NZ700688A, ES2740248T3, EP2755693A4, EP2755986A4, WO2014152940A1, EP3450553B1, BR112016030852A2, and EP3362461A1. Expression of CRISPR systems in particular is described by WO2020014577. Each of these publications are incorporated herein by reference in their entireties. Additional disclosure hereby incorporated by reference can be found in Kowalski et al., “Delivering the Messenger: Advances in Technologies for Therapeutic mRNA Delivery,” Mol Therap., 2019; 27(4): 710-728.

In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences.

In some embodiments, one or more vectors described herein are used to produce a non-human transgenic animal or transgenic plant. In some embodiments, the transgenic animal is a mammal, such as a mouse, rat, or rabbit. In certain embodiments, the organism or subject is a plant. In certain embodiments, the organism or subject or plant is algae. Methods for producing transgenic plants and animals are known in the art, and generally begin with a method of cell transfection, such as described herein.

In one aspect, the invention provides for methods of modifying a target polynucleotide in a prokaryotic or eukaryotic cell, which may be in vivo, ex vivo or in vitro. In some embodiments, the method comprises sampling a cell or population of cells from a human or non-human animal or plant (including micro-algae) and modifying the cell or cells. Culturing may occur at any stage ex vivo. The cell or cells may even be re-introduced into the non-human animal or plant (including micro-algae).

In plants, pathogens are often host-specific. For example, Fusariumn oxysporum f. sp. lycopersici causes tomato wilt but attacks only tomato, and F. oxysporum f. dianthii Puccinia graminis f. sp. tritici attacks only wheat. Plants have existing and induced defenses to resist most pathogens. Mutations and recombination events across plant generations lead to genetic variability that gives rise to susceptibility, especially as pathogens reproduce with more frequency than plants. In plants there can be non-host resistance, e.g., the host and pathogen are incompatible. There can also be Horizontal Resistance, e.g., partial resistance against all races of a pathogen, typically controlled by many genes and Vertical Resistance, e.g., complete resistance to some races of a pathogen but not to other races, typically controlled by a few genes. In a Gene-for-Gene level, plants and pathogens evolve together, and the genetic changes in one balance changes in other. Accordingly, using Natural Variability, breeders combine most useful genes for Yield. Quality, Uniformity, Hardiness, Resistance. The sources of resistance genes include native or foreign Varieties, Heirloom Varieties, Wild Plant Relatives, and Induced Mutations, e.g., treating plant material with mutagenic agents. Using the present invention, plant breeders are provided with a new tool to induce mutations. Accordingly, one skilled in the art can analyze the genome of sources of resistance genes, and in Varieties having desired characteristics or traits employ the present invention to induce the rise of resistance genes, with more precision than previous mutagenic agents and hence accelerate and improve plant breeding programs.

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

### 6.9.5. Lipid Nanoparticle Delivery

In some embodiments, the delivery system is packaged in one or more LNPs and administered intravenously. In some embodiments, the co-delivery system is packaged in one or more LNPs and administered intrathecally. In some embodiments, the co-delivery system is packaged in one or more LNPs and administered by intracerebral ventricular injection. In some embodiments, the co-delivery system is packaged in one or more LNPs and administered by intracisternal magna administration. In some embodiments, the co-delivery system is packaged in one or more LNPs and administered by intravitreal injection.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787). In some embodiments, the LNP formulations are selected from LP01 (Cas No. 1799316-64-5), ALC-0315 (Cas No. 2036272-55-4), and cKK-E12 (Cas No. 1432494-65-9). In some embodiments, the LNP formulation is LP01 (i.e., LNP #F1). In some embodiments, the LNP formulation is ALC-0315 (i.e., LNP #F2). In some embodiment, the LNP formulation is cKK-E12 (i.e., LNP #F3).

In some embodiments, LNP doses range from about 0.1 mg/kg to about 100 mg/kg (or any of the values or subranges therein). In some embodiments, LNP doses is about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, 1.5 mg/kg, about 2 mg/kg, about 2.5 mg/kg, about 3 mg/kg, about 3.5 mg/kg, about 4 mg/kg, about 4.5 mg/kg, about 5 mg/kg, about 6 mg/kg, about 7 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg or more.

In another embodiment, LNP doses of about 0.01 to about 1 mg per kg of body weight administered intravenously are contemplated. Medications to reduce the risk of infusion-related reactions are contemplated, such as dexamethasone, acetampinophen, diphenhydramine or cetirizine, and ranitidine are contemplated. Multiple doses of about 0.3 mg per kilogram every 4 weeks for five doses are also contemplated.

The charge of the LNP must be taken into consideration. As cationic lipids combined with negatively charged lipids to induce nonbilayer structures that facilitate intracellular delivery. Because charged LNPs are rapidly cleared from circulation following intravenous injection, ionizable cationic lipids with pKa values below 7 were developed (see, e.g., Rosin et al, Molecular Therapy, vol. 19, no. 12, pages 1286-2200, December 2011). Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). It has been shown that LNP siRNA systems containing these lipids exhibit remarkably different gene silencing properties in hepatocytes in vivo, with potencies varying according to the series DLinKC2-DMA>DLinKDMA>DLinDMA>>DLinDAP employing a Factor VII gene silencing model (see, e.g., Rosin et al, Molecular Therapy, vol. 19, no. 12, pages 1286-2200, December 2011). A dosage of 1 μg/ml of LNP in or associated with the LNP may be contemplated, especially for a formulation containing DLinKC2-DMA.

In some embodiments, the LNP composition comprises one or more one or more ionizable lipids. As used herein, the term “ionizable lipid” has its ordinary meaning in the art and may refer to a lipid comprising one or more charged moieties. In some embodiments, an ionizable lipid may be positively charged or negatively charged. In principle, there are no specific limitations concerning the ionizable lipids of the LNP compositions disclosed herein. In some embodiments, the one or more ionizable lipids are selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanami-ne (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3)-cholest-5-en-3-yloxy]octyl}oxy)-N,N-dimethyl-3-[(9Z,12Z)-octad-eca-9,12-dien-1-yloxy]propan-1-amine (Octyl-CLinDMA), (2R)-2-({8-[(3)-cholest-5-en-3-yloxy]octyl}oxy)-N,N-dimethyl-3-[(9Z,12Z)--octadeca-9,12-dien-1-yloxy]propan-1-amine (Octyl-CLinDMA (2R)), and (2S)-2-({8-[(3)-cholest-5-en-3-yloxy]octyl}oxy)-N,N-dimethyl-3-[(9Z,12Z)--octadeca-9,12-dien-1-y loxy]propan-1-amine (Octyl-CLinDMA (2S)). In one embodiment, the ionizable lipid may be selected from, but not limited to, an ionizable lipid described in International Publication Nos. WO2013086354 and WO2013116126.

In some embodiments, the lipid nanoparticle may include one or more (e.g., 1, 2, 3, 4, 5, 6, 7, or 8) cationic and/or ionizable lipids. Such cationic and/or ionizable lipids include, but are not limited to, 3-(didodecylamino)-N1,N1,4-tridodecyl-1-piperazineethanamine (KL10), N1-[2-(didodecylamino)ethyl]-N1,N4,N4-tridodecyl-1,4-piperazinediethanami-ne (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 2-({8-[(3.beta.)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yl oxy]propan-1-amine (Octyl-CLinDMA (2R)), (2S)-2-({8-[(33cholest-5-en-3-yloxy]octyl}oxy)-N,N-dimethyl-3-[(9Z-,12Z)-octadeca-9,12-dien-1-yl oxy]propan-1-amine (Octyl-CLinDMA (2S)).N,N-dioleyl-N,N-dimethylammonium chloride (“DODAC”); N-(2,3-dioleyloxy)propyl-N,N--N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3-.beta.-(N--(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dioleoyl-3-dimethylammonium propane (“DODAP”), N,N-dimethyl-2,3-dioleyloxy)propylamine (“DODMA”), and N-(1,2-dimyristyloxyprop-3-yl)-N,N-dimethyl-N-hydroxyethyl ammonium bromide (“DMRIE”). Additionally, a number of commercial preparations of cationic and/or ionizable lipids can be used, such as, e.g., LIPOFECTIN® (including DOTMA and DOPE, available from GIBCO/BRL), and LIPOFECTAMINE® (including DOSPA and DOPE, available from GIBCO/BRL). KL10, KL22, and KL25 are described, for example, in U.S. Pat. No. 8,691,750.

In some embodiments, the LNP composition comprises one or more amino lipids. The terms “amino lipid” and “cationic lipid” are used interchangeably herein to include those lipids and salts thereof having one, two, three, or more fatty acid or fatty alkyl chains and a pH-titratable amino head group (e.g., an alkylamino or dialkylamino head group). In principle, there are no specific limitations concerning the amino lipids of the LNP compositions disclosed herein. The cationic lipid is typically protonated (i.e., positively charged) at a pH below the pKa of the cationic lipid and is substantially neutral at a pH above the pKa. The cationic lipids can also be termed titratable cationic lipids. In some embodiments, the one or more cationic lipids include: a protonatable tertiary amine (e.g., pH-titratable) head group; alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, DSDMA, DODMA, DOTMA, DLinDMA, DLenDMA, .gamma.-DLenDMA, DLin-K-DMA, DLin-K-C2-DMA (also known as DLin-C2K-DMA, XTC2, and C2K), DLin-K-C3-DMA, DLin-K-C4-DMA, DLen-C2K-DMA, y-DLen-C2-DMA, C12-200, cKK-E12, cKK-A12, cKK-012, DLin-MC2-DMA (also known as MC2), and DLin-MC3-DMA (also known as MC3).

Anionic lipids suitable for use in lipid nanoparticles include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

Neutral lipids (including both uncharged and zwitterionic lipids) suitable for use in lipid nanoparticles include, but are not limited to, diacylphosphatidylcholine, diacylphosphatidylethanolamine, ceramide, sphingomyelin, dihydrosphingomyelin, cephalin, sterols (e.g., cholesterol) and cerebrosides. In some embodiments, the lipid nanoparticle comprises cholesterol. Lipids having a variety of acyl chain groups of varying chain length and degree of saturation are available or may be isolated or synthesized by well-known techniques. Additionally, lipids having mixtures of saturated and unsaturated fatty acid chains and cyclic regions can be used. In some embodiments, the neutral lipids used in the disclosure are DOPE, DSPC, DPPC, POPC, or any related phosphatidylcholine. In some embodiments, the neutral lipid may be composed of sphingomyelin, dihydrosphingomyeline, or phospholipids with other head groups, such as serine and inositol.

In some embodiments, amphipathic lipids are included in nanoparticles. Exemplary amphipathic lipids suitable for use in nanoparticles include, but are not limited to, sphingolipids, phospholipids, fatty acids, and amino lipids.

The lipid composition of the pharmaceutical composition may comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular amphipathic lipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural amphipathic lipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the LNP composition comprises one or more phospholipids. In some embodiments, the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16:0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine1,2-didocosahexaenoyl--sn-glycero-3-phosphoethanolamine, 1,2-dioleoyl-sn-glycero-3-phospho-rac-(1-glycerol) sodium salt (DOPG), sphingomyelin, and any mixtures thereof.

Other phosphorus-lacking compounds, such as sphingolipids, glycosphingolipid families, diacylglycerols, and .beta.-acyloxyacids, may also be used. Additionally, such amphipathic lipids can be readily mixed with other lipids, such as triglycerides and sterols.

In some embodiments, the LNP composition comprises one or more helper lipids. The term “helper lipid” as used herein refers to lipids that enhance transfection (e.g., transfection of an LNP comprising an mRNA that encodes a site-directed endonuclease, such as a SpCas9 polypeptide). In principle, there are no specific limitations concerning the helper lipids of the LNP compositions disclosed herein. Without being bound to any particular theory, it is believed that the mechanism by which the helper lipid enhances transfection includes enhancing particle stability. In some embodiments, the helper lipid enhances membrane fusogenicity. Generally, the helper lipid of the LNP compositions disclosure herein can be any helper lipid known in the art. Non-limiting examples of helper lipids suitable for the compositions and methods include steroids, sterols, and alkyl resorcinols. Particularly helper lipids suitable for use in the present disclosure include, but are not limited to, saturated phosphatidylcholine (PC) such as distearoyl-PC (DSPC) and dipalymitoyl-PC (DPPC), dioleoylphosphatidylethanolamine (DOPE), 1,2-dilinoleoyl-sn-glycero-3-phosphocholine (DLPC), cholesterol, 5-heptadecylresorcinol, and cholesterol hemisuccinate. In some embodiments, the helper lipid of the LNP composition includes cholesterol.

In some embodiments, the LNP composition comprises one or more structural lipids. As used herein, the term “structural lipid” refers to sterols and also to lipids containing sterol moieties. Without being bound to any particular theory, it is believed that the incorporation of structural lipids into the LNPs mitigates aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In some embodiments, the structural lipid is cholesterol. In certain embodiments, the structural lipid is an analog of cholesterol.

The lipid component of a lipid nanoparticle composition may include one or more molecules comprising polyethylene glycol, such as PEG or PEG-modified lipids. In some embodiments, the LNP composition disclosed herein comprise one or more polyethylene glycol (PEG) lipid. The term “PEG-lipid” refers to polyethylene glycol (PEG)-modified lipids. Such lipids are also referred to as PEGylated lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines For example, a PEG lipid can be PEG-c-DOMG, PEG-DMG, PEG-DLPE, PEG-DMPE, PEG-DPPC, or a PEG-DSPE lipid. In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the lipid moiety of the PEG-lipids includes those having lengths of from about C.sub.14 to about C.sub.22, preferably from about C.sub.14 to about C.sub.16. In some embodiments, a PEG moiety, for example a mPEG-NH.sub.2, has a size of about 1000, 2000, 5000, 10,000, 15,000 or 20,000 daltons. In some embodiment, the PEG-lipid is PEG2k-DMG. In some embodiments, the one or more PEG lipids of the LNP composition comprises PEG-DMPE. In some embodiments, the one or more PEG lipids of the LNP composition comprises PEG-DMG.

In some embodiments, the ratio between the lipid components and the nucleic acid molecules of the LNP composition, e.g., the weight ratio, is sufficient for (i) formation of LNPs with desired characteristics, e.g., size, charge, and (ii) delivery of a sufficient dose of nucleic acid at a dose of the lipid component(s) that is tolerable for in vivo administration as readily ascertained by one of skill in the art.

In certain embodiments, it is desirable to target a nanoparticle, e.g., a lipid nanoparticle, using a targeting moiety that is specific to a cell type and/or tissue type. In some embodiments, a nanoparticle may be targeted to a particular cell, tissue, and/or organ using a targeting moiety. In particular embodiments, a nanoparticle comprises a targeting moiety. Exemplary non-limiting targeting moieties include ligands, cell surface receptors, glycoproteins, vitamins (e.g., riboflavin) and antibodies (e.g., full-length antibodies, antibody fragments (e.g., Fv fragments, single chain Fv (scFv) fragments, Fab′ fragments, or F(ab′)2 fragments), single domain antibodies, camelid antibodies and fragments thereof, human antibodies and fragments thereof, monoclonal antibodies, and multispecific antibodies (e.g., bispecific antibodies)). In some embodiments, the targeting moiety may be a polypeptide. The targeting moiety may include the entire polypeptide (e.g., peptide or protein) or fragments thereof. A targeting moiety is typically positioned on the outer surface of the nanoparticle in such a manner that the targeting moiety is available for interaction with the target, for example, a cell surface receptor. A variety of different targeting moieties and methods are known and available in the art, including those described, e.g., in Sapra et al., Prog. Lipid Res. 42(5):439-62, 2003 and Abra et al., J. Liposome Res. 12:1-3, 2002.

In some embodiments, a lipid nanoparticle (e.g., a liposome) may include a surface coating of hydrophilic polymer chains, such as polyethylene glycol (PEG) chains (see, e.g., Allen et al., Biochimica et Biophysica Acta 1237: 99-108, 1995; DeFrees et al., Journal of the American Chemistry Society 118: 6101-6104, 1996; Blume et al., Biochimica et Biophysica Acta 1149: 180-184,1993; Klibanov et al., Journal of Liposome Research 2: 321-334, 1992; U.S. Pat. No. 5,013,556; Zalipsky, Bioconjugate Chemistry 4: 296-299, 1993; Zalipsky, FEBS Letters 353: 71-74, 1994; Zalipsky, in Stealth Liposomes Chapter 9 (Lasic and Martin, Eds) CRC Press, Boca Raton Fla., 1995). In one approach, a targeting moiety for targeting the lipid nanoparticle is linked to the polar head group of lipids forming the nanoparticle. In another approach, the targeting moiety is attached to the distal ends of the PEG chains forming the hydrophilic polymer coating (see, e.g., Klibanov et al., Journal of Liposome Research 2: 321-334, 1992; Kirpotin et al., FEBS Letters 388: 115-118, 1996).

Standard methods for coupling the targeting moiety or moieties may be used. For example, phosphatidylethanolamine, which can be activated for attachment of targeting moieties, or derivatized lipophilic compounds, such as lipid-derivatized bleomycin, can be used. Antibody-targeted liposomes can be constructed using, for instance, liposomes that incorporate protein A (see, e.g., Renneisen et al., J. Bio. Chem., 265:16337-16342, 1990 and Leonetti et al., Proc. Natl. Acad. Sci. (USA), 87:2448-2451, 1990). Other examples of antibody conjugation are disclosed in U.S. Pat. No. 6,027,726. Examples of targeting moieties can also include other polypeptides that are specific to cellular components, including antigens associated with neoplasms or tumors. Polypeptides used as targeting moieties can be attached to the liposomes via covalent bonds (see, for example Heath, Covalent Attachment of Proteins to Liposomes, 149 Methods in Enzymology 111-119 (Academic Press, Inc. 1987)). Other targeting methods include the biotin-avidin system.

In some embodiments, a lipid nanoparticle includes a targeting moiety that targets the lipid nanoparticle to a cell including, but not limited to, hepatocytes, colon cells, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells (including primary tumor cells and metastatic tumor cells). In particular embodiments, the targeting moiety targets the lipid nanoparticle to a hepatocyte.

The lipid nanoparticles described herein may be lipidoid-based. The synthesis of lipidoids has been extensively described and formulations containing these compounds are particularly suited for delivery of polynucleotides (see Mahon et al., Bioconjug Chem. 2010 21:1448-1454; Schroeder et al., J Intern Med. 2010 267:9-21; Akinc et al., Nat. Biotechnol. 2008 26:561-569; Love et al., Proc Natl Acad Sci USA. 2010 107:1864-1869; Siegwart et al., Proc Natl Acad Sci USA. 2011 108:12996-3001).

The characteristics of optimized lipidoid formulations for intramuscular or subcutaneous routes may vary significantly depending on the target cell type and the ability of formulations to diffuse through the extracellular matrix into the blood stream. While a particle size of less than 150 nm may be desired for effective hepatocyte delivery due to the size of the endothelial fenestrae (see e.g., Akinc et al., Mol Ther. 2009 17:872-879), use of lipidoid oligonucleotides to deliver the formulation to other cells types including, but not limited to, endothelial cells, myeloid cells, and muscle cells may not be similarly size-limited.

In one aspect, effective delivery to myeloid cells, such as monocytes, lipidoid formulations may have a similar component molar ratio. Different ratios of lipidoids and other components including, but not limited to, a neutral lipid (e.g., diacylphosphatidylcholine), cholesterol, a PEGylated lipid (e.g., PEG-DMPE), and a fatty acid (e.g., an omega-3 fatty acid) may be used to optimize the formulation of the mRNA or system for delivery to different cell types including, but not limited to, hepatocytes, myeloid cells, muscle cells, etc. Exemplary lipidoids include, but are not limited to, DLin-DMA, DLin-K-DMA, DLin-KC2-DMA, 98N12-5, C12-200 (including variants and derivatives), DLin-MC3-DMA and analogs thereof. The use of lipidoid formulations for the localized delivery of nucleic acids to cells (such as, but not limited to, adipose cells and muscle cells) via either subcutaneous or intramuscular delivery, may also not require all of the formulation components which may be required for systemic delivery, and as such may comprise the lipidoid and the mRNA or system.

According to the present disclosure, a system described herein may be formulated by mixing the mRNA or system, or individual components of the system, with the lipidoid at a set ratio prior to addition to cells. In vivo formulations may require the addition of extra ingredients to facilitate circulation throughout the body. After formation of the particle, a system or individual components of a system is added and allowed to integrate with the complex. The encapsulation efficiency is determined using a standard dye exclusion assays.

In vivo delivery of systems may be affected by many parameters, including, but not limited to, the formulation composition, nature of particle PEGylation, degree of loading, oligonucleotide to lipid ratio, and biophysical parameters such as particle size (Akinc et al., Mol Ther. 2009 17:872-879; herein incorporated by reference in its entirety). As an example, small changes in the anchor chain length of poly(ethylene glycol) (PEG) lipids may result in significant effects on in vivo efficacy. Formulations with the different lipidoids, including, but not limited to penta[3-(1-laurylaminopropionyl)]-triethylenetetramine hydrochloride (TETA-5LAP; aka 98N12-5, see Murugaiah et al., Analytical Biochemistry, 401:61 (2010)), C12-200 (including derivatives and variants), MD1, DLin-DMA, DLin-K-DMA, DLin-KC2-DMA and DLin-MC3-DMA can be tested for in vivo activity. The lipidoid referred to herein as “98N12-5” is disclosed by Akinc et al., Mol Ther. 2009 17:872-879). The lipidoid referred to herein as “C12-200” is disclosed by Love et al., Proc Natl Acad Sci USA. 2010 107:1864-1869 and Liu and Huang, Molecular Therapy. 2010 669-670.

The LNPs of the present disclosure, in which a nucleic acid is entrapped within the lipid portion of the particle and is protected from degradation, can be formed by any method known in the art including, but not limited to, a continuous mixing method, a direct dilution process, and an in-line dilution process. Additional techniques and methods suitable for the preparation of the LNPs described herein include coacervation, microemulsions, supercritical fluid technologies, phase-inversion temperature (PIT) techniques.

In some embodiments, the LNPs used herein are produced via a continuous mixing method, e.g., a process that includes providing an aqueous solution a nucleic acid described herein in a first reservoir, providing an organic lipid solution in a second reservoir (wherein the lipids present in the organic lipid solution are solubilized in an organic solvent, e.g., a lower alkanol such as ethanol), and mixing the aqueous solution with the organic lipid solution such that the organic lipid solution mixes with the aqueous solution so as to substantially instantaneously produce a lipid vesicle (e.g., liposome) encapsulating the nucleic acid molecule within the lipid vesicle. This process and the apparatus for carrying out this process are known in the art. More information in this regard can be found in, for example, U.S. Patent Publication No. 20040142025. The action of continuously introducing lipid and buffer solutions into a mixing environment, such as in a mixing chamber, causes a continuous dilution of the lipid solution with the buffer solution, thereby producing a lipid vesicle substantially instantaneously upon mixing. By mixing the aqueous solution comprising a nucleic acid molecule with the organic lipid solution, the organic lipid solution undergoes a continuous stepwise dilution in the presence of the buffer solution (e.g., aqueous solution) to produce a nucleic acid-lipid particle.

In some embodiments, the LNPs used herein are produced via a direct dilution process that includes forming a lipid vesicle (e.g., liposome) solution and immediately and directly introducing the lipid vesicle solution into a collection vessel containing a controlled amount of dilution buffer. In some embodiments, the collection vessel includes one or more elements configured to stir the contents of the collection vessel to facilitate dilution. In some embodiments, the amount of dilution buffer present in the collection vessel is substantially equal to the volume of lipid vesicle solution introduced thereto.

In some embodiments, the LNPs are produced via an in-line dilution process in which a third reservoir containing dilution buffer is fluidly coupled to a second mixing region. In these embodiments, the lipid vesicle (e.g., liposome) solution formed in a first mixing region is immediately and directly mixed with dilution buffer in the second mixing region. These processes and the apparatuses for carrying out direct dilution and in-line dilution processes are known in the art. More information in this regard can be found in, for example, U.S. Patent Publication No. 20070042031.

### 6.10. Genes and Targets

This disclosure provides compositions and co-delivery methods for correcting or replacing genes or gene fragments (including introns or exons) or inserting genes in new locations. In certain embodiments, such a method comprises recombination or integration into a safe harbor site (SHS). A frequently used human SHS is the AAVS1 site on chromosome 19q, initially identified as a site for recurrent adeno-associated virus insertion. Another locus comprises the human homolog of the murine Rosa26 locus. Yet another SHS comprises the human H11 locus on chromosome 22. In some cases, a complete gene may be prohibitively large and replacement of an entire gene impractical. In certain embodiments, a method of the invention comprises recombining corrective gene fragments into a defective locus.

The methods and compositions can be used to target, without limitation, stem cells for example induced pluripotent stem cells (iPSCs), HSCs, HSPCs, mesenchymal stem cells, or neuronal stem cells and cells at various stages of differentiation. In certain embodiments, methods and compositions of the invention are adapted to target organoids, including patient derived organoids.

In certain embodiments, methods and compositions of the invention are adapted to treat muscle cells, not limited to cardiomyocytes for Duchene Muscular Dystrophy (DMD). The dystrophin gene is the largest gene in the human genome, spanning ˜2.3 Mb of DNA. DMD is composed of 79 exons resulting in a 14-kb full-length mRNA. Common mutations include mutations that disrupt the reading frame of generate a premature stop codon. An aspect of DMD that lends it to gene editing as a therapeutic approach is the modular structure of the dystrophin protein. Redundancy in the central rod domain permits the deletion of internal segments of the gene that may harbor loss-of-function mutations, thereby restoring the open reading frame (ORFs). In some embodiments, the methods and systems described herein are used to treat DMD by site-specifically integrating in the genome a polynucleotide template that repairs or replaces all or a portion of the defective DMD gene.

The following are non limiting diseases that may be treated utilizing the methods and compositions of the present disclosure:

### Inherited Retinal Diseases:

Stargardt Disease (ABCA4)
Leber congenital amaurosis 10 (CEP290)
X linked Retinitis Pigmentosa (RPGR)
Autosomal Dominant Retinitis Pigmentosa (RHO)

### Liver Diseases:

Wilson's disease (ATP7B)
Alpha-1 antitrypsin (SERPINA1)

### Intellectual Disabilities:

Rett Syndrome (MECP2)
SYNGAP1-ID (SYNGAP1)
CDKL5 deficiency disorder (CDKL5)

### Peripheral Neuropathies:

Charcot-Marie-Tooth 2A (MFN2)

### Lung Diseases:

Cystic Fibrosis (CFTR)
Alpha-1 Antitrypsin (SERPINA1)

### Blood Disorders:

Sickle Cell
Hemophilia,
Factor VIII or
Factor IX
CFTR (cystic fibrosis transmembrane conductance regulator)

Over 2500 mutations have been identified associated with various diseases and defects.

The most common cystic fibrosis (CF) mutation F508del removes a single amino acid. In some embodiments, recombining human CFTR into an SHS of a cell that expresses CFTR F508del is a corrective treatment path. In some embodiments, the methods and systems described herein are used to CF by site-specifically integrating in the genome a polynucleotide template that corrects the mutation causing CF. Proposed validation is detection of persistent CFTR mRNA and protein expression in transduced cells.

Sickle cell disease (SCD) is caused by mutation of a specific amino acid—valine to glutamic acid at amino acid position 6. In some embodiments, SCD is corrected by recombination of the HBB gene into a safe harbor site (SHS) and by demonstrating correction in a proportion of target cells that is high enough to produce a substantial benefit. In some embodiments, the methods and systems described herein are used to sickle cell disease by site-specifically integrating in the genome a polynucleotide template that corrects the mutation causing the disease. In some embodiments, validation is detection of persistent HBB mRNA and protein expression in transduced cells.

### DMD—Duchenne Muscular Dystrophy

The dystrophin gene is the largest gene in the human genome, spanning ˜2.3 Mb of DNA. DMD is composed of 79 exons resulting in a 14-kb full-length mRNA. Common mutations include mutations that disrupt the reading frame of generate a premature stop codon. An aspect of DMD that lends it to gene editing as a therapeutic approach is the modular structure of the dystrophin protein. Redundancy in the central rod domain permits the deletion of internal segments of the gene that may harbor loss-of-function mutations, thereby restoring the open reading frame (ORFs).

In some embodiments, recombination will be into safe harbor sites (SHS). A frequently used human SHS is the AAVS1 site on chromosome 19q, initially identified as a site for recurrent adeno-associated virus insertion. In some embodiments, the site is the human homolog of the e murine Rosa26 locus (pubmed.ncbi.nlm.nih.gov/18037879). In some embodiments, the site is the human H11 locus on chromosome 22. Proposed target cells for recombination include stem cells for example induced pluripotent stem cells (iPSCs) and cells at various stages of differentiation. In some cases, a complete gene may be prohibitively large and replacement of an entire gene impractical. In such instances, rescuing mutants by recombining in corrected gene fragments with the methods and systems described herein is a corrective option.

In some embodiments, correcting mutations in exon 44 (or 51) by recombining in a corrective coding sequence downstream of exon 43 (or 50), using the methods and systems described herein is a corrective option. Proposed validation is detection of persistent DMD mRNA and protein expression in transduced cells.

### F8 (Factor VIII)

A large proportion of severe hemophilia A patients harbor one of two types of chromosomal inversions in the FVIII gene. The recombinase technology and methods described herein are well suited to correcting such inversions (and other mutations) by recombining of the FVIII gene into a SHS.

In some embodiments, correcting factor VIII deficiency by recombining the FVIII gene into an SHS is a corrective path. In some embodiments, the methods and systems described herein are used to correct factor VIII deficiency by site-specifically integrating in the genome a polynucleotide template that corrects the mutation causing the FIX deficiency. Proposed validation is detection of persistent FVIII mRNA and protein expression in transduced cells.

### Factor 9 (Factor IX)

Hemophilia B, also called factor IX (FIX) deficiency is a genetic disorder caused by missing or defective factor IX, a clotting protein.

In some embodiments, the methods and systems described herein are used to correct factor IX deficiency by site-specifically integrating in the genome a polynucleotide template that corrects the mutation causing the FIX deficiency. Proposed validation is detection of persistent FiX mRNA and protein expression in transduced cells.

### 6.11. Methods of Treatment

In another aspect, methods of treatment are presented. The method comprises administering an effective amount of the pharmaceutical composition comprising the nucleic acid construct or vectorized nucleic acid construct described above to a patient in need thereof. In some embodiments, the system (e.g., any of the systems described herein) are delivered to a cell ex vivo and the cell is then administered to the subject. In some embodiments, the systems (e.g., any of the systems described herein) are delivered to a patient, thereby delivering to a cell in vivo.

DNA or RNA viral vectors can be administered directly to patients (in vivo) or they can be used to treat cells in vitro, and the modified cells may optionally be administered to patients (ex vivo). Conventional viral based systems to be used herein could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

In some embodiments, the co-delivery system described herein (e.g., a gene editor construct packaged in a LNP and a donor template packaged in a vector) is administered intravenously. In some embodiments, the co-delivery system described herein (e.g., a gene editor construct packaged in a LNP and a donor template packaged in a vector) is administered intrathecally. In some embodiments, the co-delivery system described herein (e.g., a gene editor construct packaged in a LNP and a donor template packaged in a vector) is administered by intracerebral ventricular injection. In some embodiments, the co-delivery system described herein (e.g., a gene editor construct packaged in a LNP and a donor template packaged in a vector) is administered by intracisternal magna administration. In some embodiments, the co-delivery system described herein (e.g., a gene editor construct packaged in a LNP and a donor template packaged in a vector) is administered by intravitreal injection.

Methods of non-viral delivery of the donor DNA template described herein include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

6.11.1.1 mRNA Delivery

Another useful method to deliver proteins, enzymes, and guides comprises transfection of messenger RNA (mRNA). Examples of mRNA delivery methods and compositions that may be utilized in the present disclosure including, for example, PCT/US2014/028330, U.S. Pat. No. 8,822,663B2, NZ700688A, ES2740248T3, EP2755693A4, EP2755986A4, WO2014152940A1, EP3450553B1, BR112016030852A2, and EP3362461A1. Expression of CRISPR systems in particular is described by WO2020014577. Each of these publications are incorporated herein by reference in their entireties. Additional disclosure hereby incorporated by reference can be found in Kowalski et al., “Delivering the Messenger: Advances in Technologies for Therapeutic mRNA Delivery,” Mol Therap., 2019; 27(4): 710-728.

### 6.12. Additional Embodiments

Embodiment 1. A method of co-delivering to a cell a gene editor polynucleotide construct and a template polynucleotide construct, the method comprising co-delivering:
a lipid nanoparticle (LNP) comprising a gene editor polynucleotide construct; and
a vector comprising a donor template polynucleotide construct.

Embodiment 2. The method of embodiment 1, wherein the gene editor polynucleotide construct is capable of localizing to a cell cytoplasm.

Embodiment 3. The method of embodiment 1, wherein the donor template polynucleotide construct is capable of localizing to a cell nucleus.

Embodiment 4. The method of embodiment 1 or embodiment 2, wherein the gene editor polynucleotide construct comprises:
a polynucleotide sequence encoding a prime editor fusion protein or a Gene Writer™ protein;
a one or more polynucleotide sequence encoding an attachment site-containing guide RNA (atgRNA);
optionally, a polynucleotide sequence encoding a nickase guide RNA (ngRNA);
a polynucleotide sequence encoding an integrase;
optionally, a polynucleotide sequence encoding a recombinase.

Embodiment 5. The method of embodiment 4, wherein the integrase that is encoded by a polynucleotide sequence in the gene editor polynucleotide construct is fused to the prime editor fusion protein or the Gene Writer™ protein encoded by a gene editor polynucleotide construct, and wherein the fusion is optionally by a linker.

Embodiment 6. The method of any of embodiment 4 or embodiment 5, wherein the one or more atgRNA encodes an integrase target recognition side or a recombinase recognition site.

Embodiment 7. The method of any of the previous embodiments, wherein the vector comprising a donor template polynucleotide construct, the vector is recombinant adenovirus, helper dependent adenovirus, AAV, lentivirus, HSV, annelovirus, retrovirus, Doggybone™ DNA (dbDNA), minicircle, plasmid, miniDNA, exosome, fusosome, or nanoplasmid.

Embodiment 8. The method of any of the previous embodiments, wherein the donor template is capable of being integrated into a genomic locus that contains an integrase target recognition site or a recombinase target integrase site.

Embodiment 9. The method of any of the previous embodiments, wherein the donor template comprises at least one of the following: a gene, a gene fragment, an expression cassette, a logic gate system, or any combination thereof.

Embodiment 10. The method of any of the previous embodiments, wherein the donor template further comprises at least one integrase target recognition site or a recombinase target integrase site.

Embodiment 11. The method of any of the previous embodiments, wherein the donor template is capable of self-circularization to form a circularized nucleic acid.

Embodiment 12. The circularized nucleic acid of embodiment 11, wherein the self-circularizing is mediated by an integrase or recombinase.

Embodiment 13. A pharmaceutical co-delivery composition comprising:
(a) a lipid nanoparticle (LNP) comprising a gene editor polynucleotide construct (i) capable of localizing to a cell cytoplasm; and
(b) a vector comprising a donor template polynucleotide construct (ii) capable of localizing to a cell nucleus.

Embodiment 14. A pharmaceutical co-delivery composition of embodiment 13, wherein the gene editor polynucleotide construct comprises:
a polynucleotide sequence encoding a prime editor fusion protein or a Gene Writer™ protein;
a polynucleotide sequence encoding an attachment site-containing guide RNA;
optionally, a polynucleotide sequence encoding a nickase guide RNA (ngRNA);
a polynucleotide sequence encoding an integrase;
optionally, a polynucleotide sequence encoding a recombinase; and
wherein the donor template polynucleotide construct is packaged in recombinant adenovirus, helper dependent adenovirus, AAV, lentivirus, HSV, annelovirus, retrovirus, Doggybone DNA (dbDNA), minicircle, plasmid, miniDNA, exsosome, fusosome, or nanoplasmid.

Embodiment 15. A method comprising administering an effective amount of the pharmaceutical composition of embodiment 13 or embodiment 14, to a patient in need thereof.

### 7. EXAMPLES

### 7.1. Example 1: Delivery of Gene Editor Polynucleotide Sequence Packaged in LNP and Donor Template Packaged in AAV

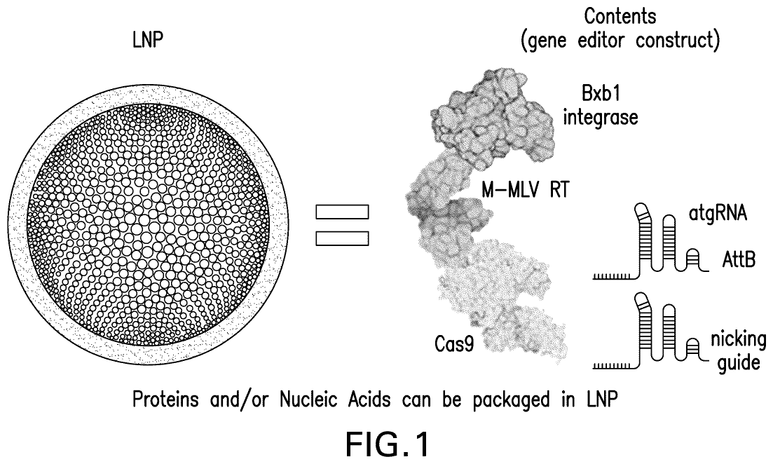

A gene editor polynucleotide construct is packaged into a LNP (FIG. 1), wherein the gene editor polynucleotide sequence comprises a polynucleotide sequence encoding a prime editor protein linked to an integrase via peptide linker a polynucleotide sequence encoding an attachment site-containing guide RNA (atgRNA); a polynucleotide sequence encoding a nickase guide RNA (ngRNA).

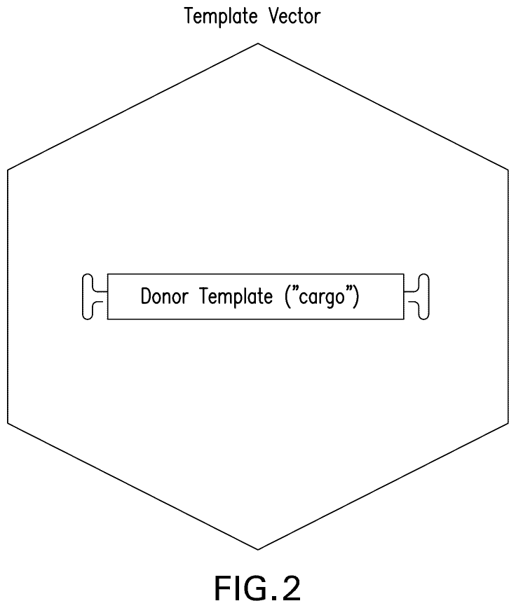

A donor template polynucleotide construct is packaged in an AAV vector (FIG. 2).

Co-administration of the gene editor construct packaged LNP and the donor template packaged AAV co-delivers the gene editor construct to a cell cytoplasm and the donor template to a cell nucleus. By use of programmable genome editing to place integrase landing site at a desired location in the genome, the direct activity of the associated integrase to the specific genomic site is guided. Gene editor construct expression, with template co-delivery, results in integration of template “cargo” at a precisely defined target location.

### 7.2. Example 2: Delivery of Gene Editor Polynucleotide Sequence Packaged in LNP and Donor Template Capable of Self-Circularization Packaged in AAV

A gene editor polynucleotide construct is packaged into a LNP (FIG. 1), wherein the gene editor polynucleotide sequence comprises a polynucleotide sequence encoding a prime editor protein linked to an integrase via peptide linker a polynucleotide sequence encoding an attachment site-containing guide RNA (atgRNA); a polynucleotide sequence encoding a nickase guide RNA (ngRNA).

A donor template polynucleotide construct is packaged in an AAV vector (FIG. 2).

Co-administration of the gene editor construct packaged LNP and the donor template packaged AAV co-delivers the gene editor construct to a cell cytoplasm and the donor template to a cell nucleus. Integrase-mediated self-circularization of donor template occurs at integration target recognition sites within the AAV genome (FIG. 3). By use of programmable genome editing to place an orthogonal integrase landing site (i.e., distinct att site from att sites used for self-circularization) at a desired location in the genome, the direct activity of the associated integrase to the specific genomic site is guided. Gene editor construct expression, with template co-delivery and integrase-mediated circularization of template, results in integration of template “cargo” at a precisely defined target location.

### 7.3. Example 3: Delivery of Gene Editor Polynucleotide Sequence Packaged in LNP and atgRNA, ngRNA, and Donor Template Co-Packaged in AAV

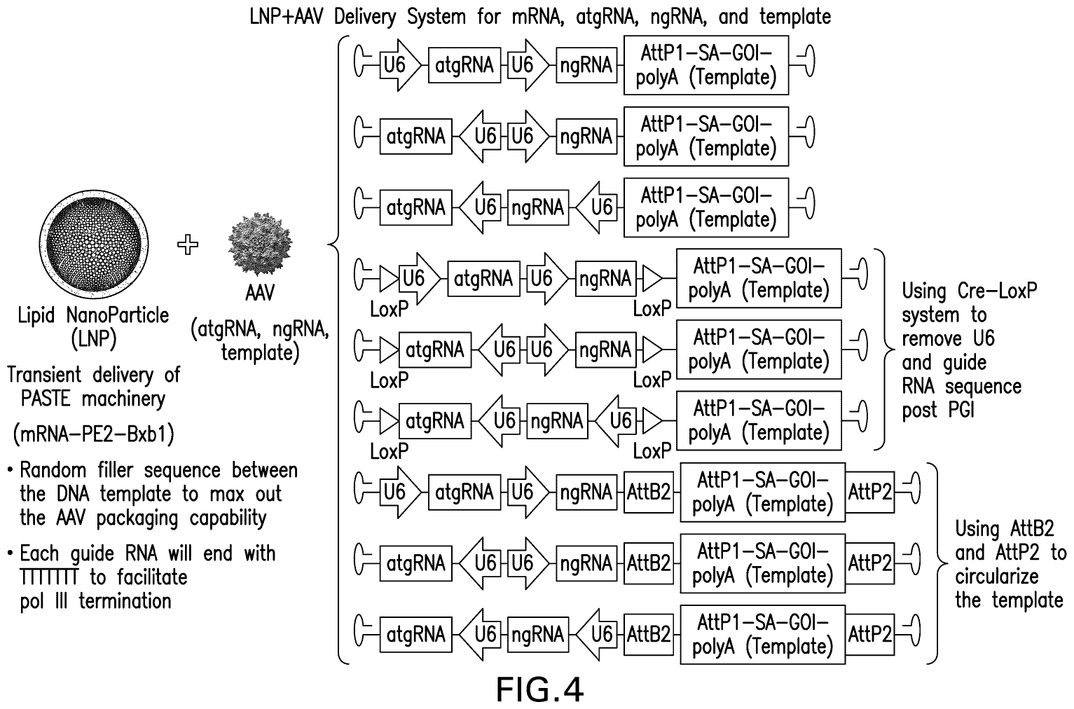

A gene editor polynucleotide construct is packaged into a LNP (FIG. 4), wherein the gene editor polynucleotide sequence comprises a polynucleotide sequence encoding a prime editor protein linked to an integrase via peptide linker.

A polynucleotide sequence encoding an attachment site-containing guide RNA (atgRNA), a polynucleotide sequence encoding a nicking guide RNA (ngRNA), and donor template are packaged in an AAV vector (FIG. 4).

Co-administration of the gene editor construct packaged LNP and the atgRNA, ngRNA, donor template packaged AAV co-delivers the gene editor construct to a cell. Integrase-mediated self-circularization of donor template occurs at integration target recognition sites within the AAV genome (FIG. 3). By use of programmable genome editing to place an orthogonal integrase landing site (i.e., distinct att site from att sites used for self-circularization) at a desired location in the genome, the direct activity of the associated integrase to the specific genomic site is guided. Gene editor construct expression, with atgRNA, ngRNA, and template co-delivery and integrase-mediated circularization of template, results in integration of template “cargo” at a precisely defined target location.

### 7.4. Example 4: Delivery of Gene Editor Polynucleotide Sequence and ngRNA Packaged in LNP and atgRNA and Donor Template Co-Packaged in AAV

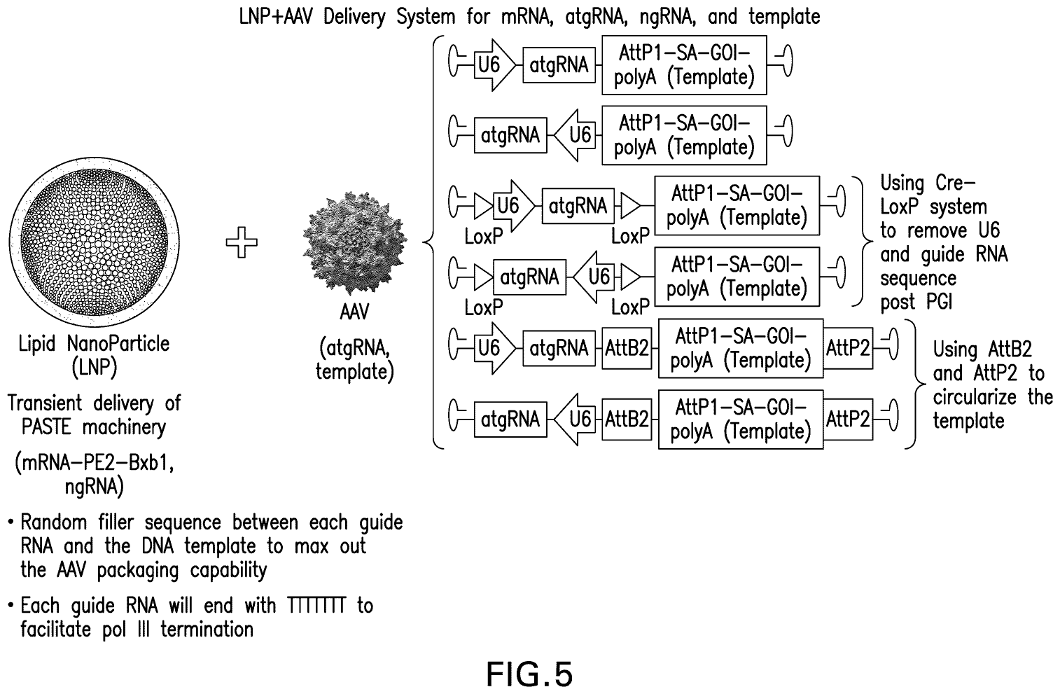

A gene editor polynucleotide construct and a nicking guide RNA (ngRNA) are packaged into a LNP (FIG. 5), wherein the gene editor polynucleotide sequence comprises a polynucleotide sequence encoding a prime editor protein linked to an integrase via peptide linker.

A polynucleotide sequence encoding an attachment site-containing guide RNA (atgRNA) and donor template are packaged in an AAV vector (FIG. 5).

Co-administration of the gene editor construct and ngRNA packaged LNP and the atgRNA, donor template packaged AAV co-delivers the gene editor construct to a cell. Integrase-mediated self-circularization of donor template occurs at integration target recognition sites within the AAV genome (FIG. 3). By use of programmable genome editing to place an orthogonal integrase landing site (i.e., distinct att site from att sites used for self-circularization) at a desired location in the genome, the direct activity of the associated integrase to the specific genomic site is guided. Gene editor construct expression, with atgRNA, ngRNA, and template co-delivery and integrase-mediated circularization of template, results in integration of template “cargo” at a precisely defined target location.

### 7.5. Example 5: Intramolecular Circularization of Plasmid and Packaged AAV Genomes

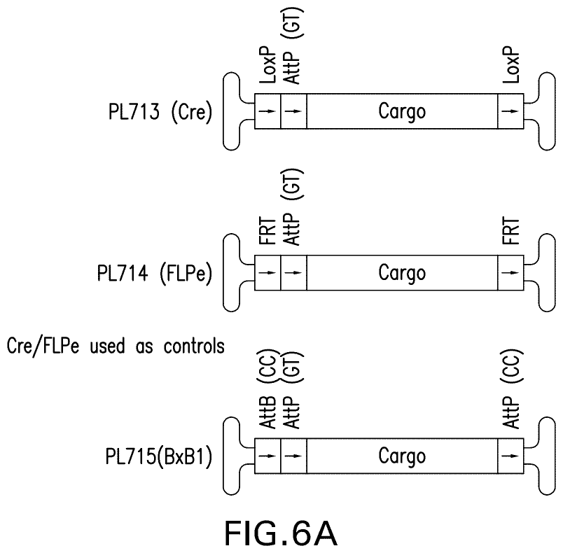

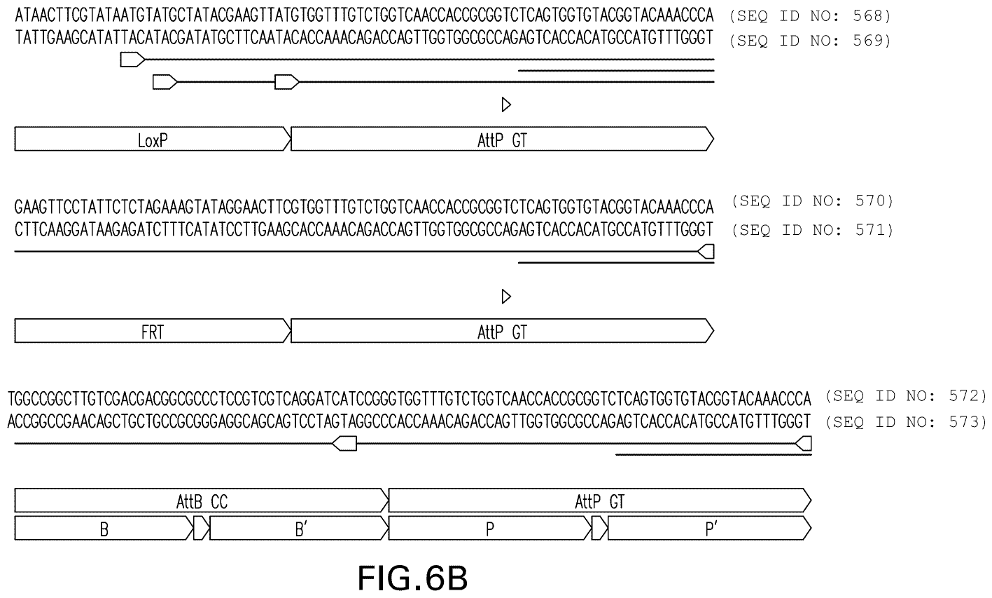

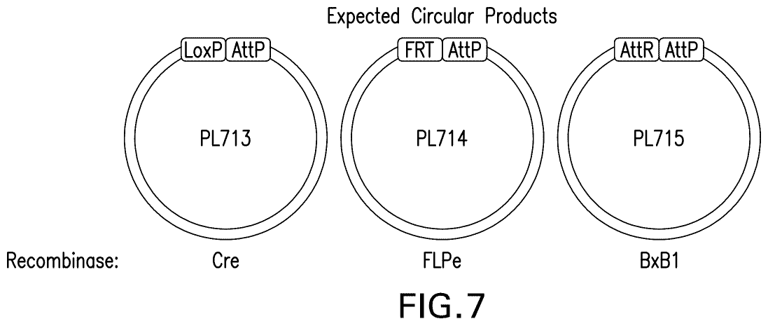

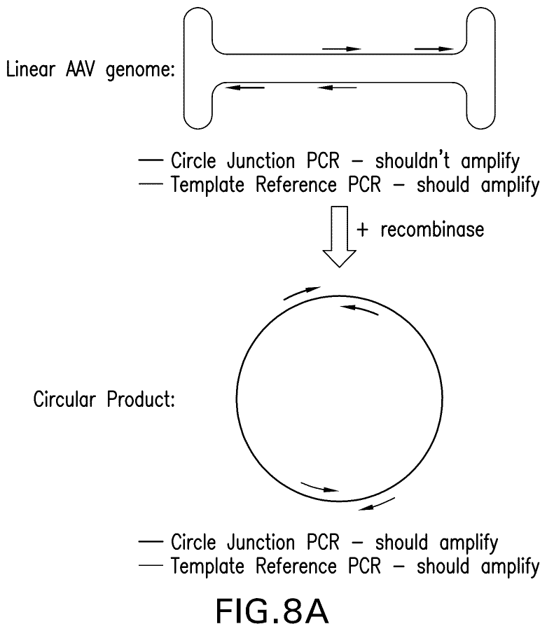

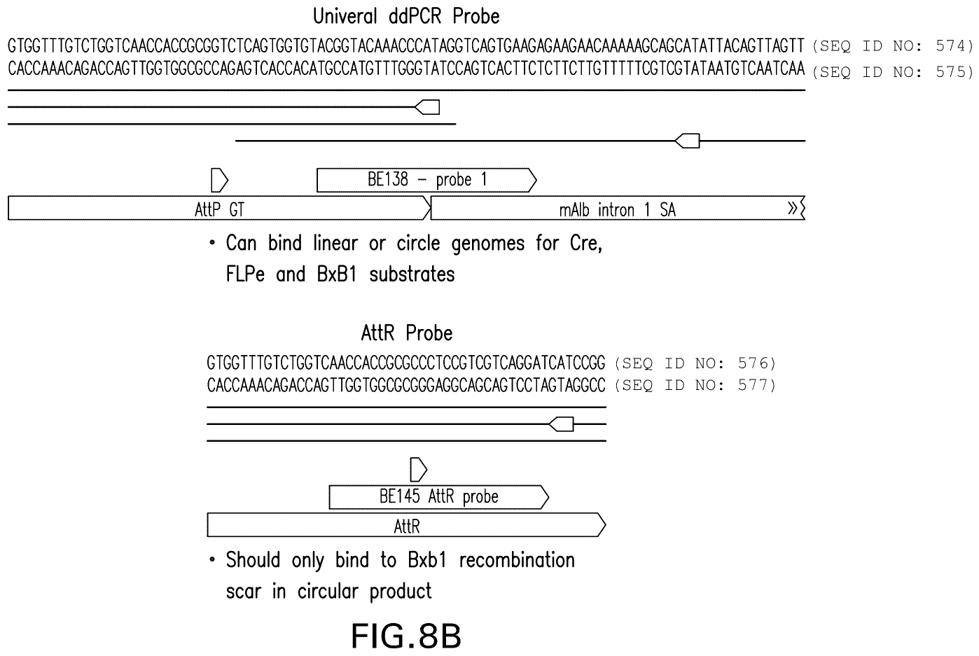

Three self-complementary AAV (scAAV) genomes were designed and generated to verify recombinase/integrase-mediated intramolecular circularization of a DNA cargo from within a linear AAV genome (FIGS. 6A-6B). Circularization of a scAAV genome is mediated by one of Cre, FLPe (thermostable mutant), or Bxb1. Further, the scAAV genomes are comprised of a DNA cargo of interest (“payload”) and an attP site (GT central dinucleotide for circularization orthogonality) for gene insertion into a genome placed attB beacon site. Expected recombinase/integrase-mediated intramolecular circularization products are illustrated in FIG. 7. A universal ddPCR probe capable of binding any linear or circularized AAV genome was designed, wherein the universal ddPCR probe is designed to only give signal upon cognate recombinase/integrase mediated circularization (FIGS. 8A-8B). Circularization products are amplified by use of a circle junction PCR primer set that is designed to amplify only circular products due to primer direction constraints. To confirm Bxb1 mediated circularization specifically, an attR scar quencher-fluorophore probe was designed. In addition, a template reference primer set was designed and generated to quantify total template DNA (linear or circular confirmation) (FIGS. 8A-8B).

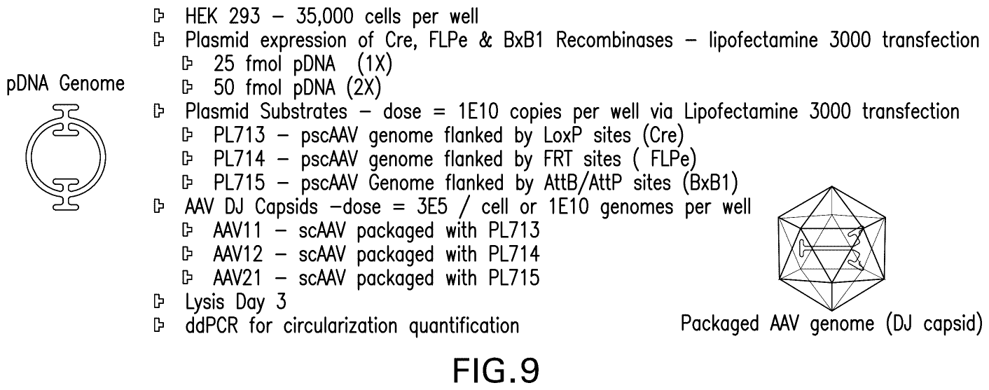

Intracellular circularization of either plasmid or packaged AAV genomes were screened in HEK293 cells (35K cells per well) (FIG. 9). Plasmids (25 fmol pDNA=1× or 50 fmol pDNA=2×) encoding one of Cre, FLPe, or Bxb1 were transfected by Lipofectamine 3000. Plasmid genome substrates were transfected at a dose of 1E10 copies per well using Lipofectamine 3000 (FIG. 9). Additionally, AAV genomes were packaged in AAV-DJ capsids and delivered at a dose of 3E5 genomes per cell or 1E10 genomes per well. Circularization ddPCR analysis was conducted three days post transfection.

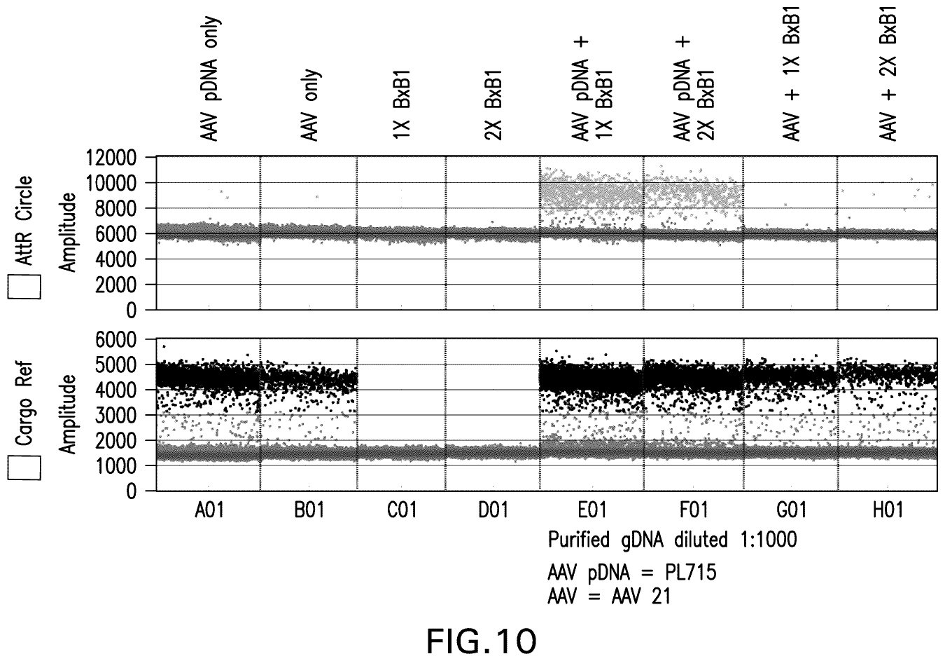

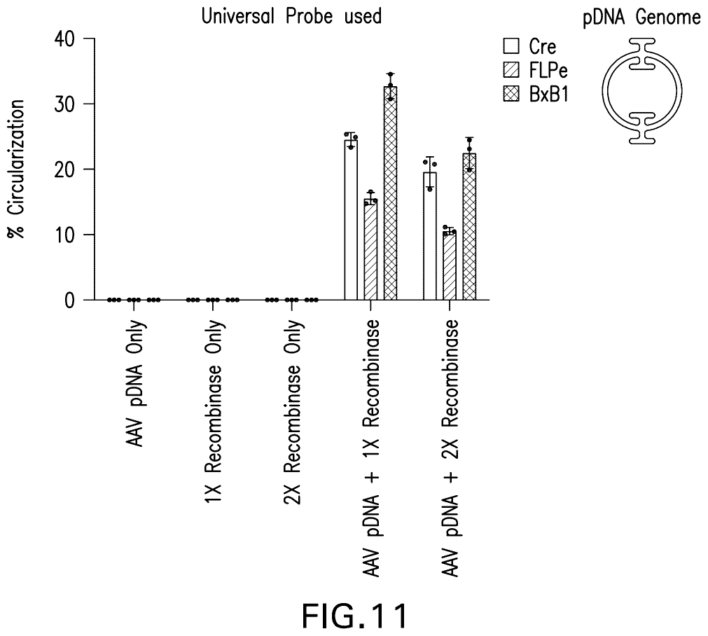

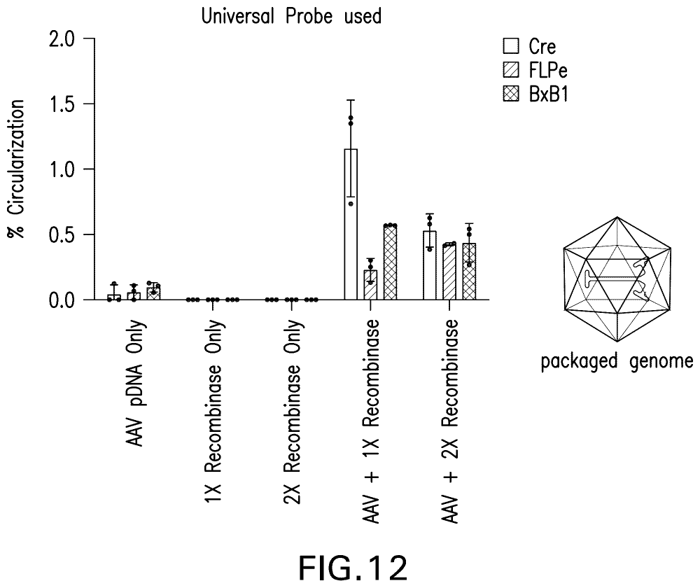

FIG. 10 demonstrates circularization of AAV pDNA and packaged AAV genomic DNA for both 1×Bxb1 and 2×Bxb1 conditions (confirmed by use of attR ddPCR primer set). Further, replicates that lacked either Bxb1 or AAV pDNA substrate demonstrated insignificant circularization. All three of the Cre-, FLPe-, and Bxb1-targeted AAV pDNA substrates demonstrated circularization upon cognate recombinase/integrase introduction, as confirmed by using the universal ddPCR probe (FIG. 11). Moreover, Cre-, FLPe-, and Bxb1-mediated circularization of packaged AAV DJ genomes substrates were demonstrated and confirmed using the universal ddPCR probe (FIG. 12).

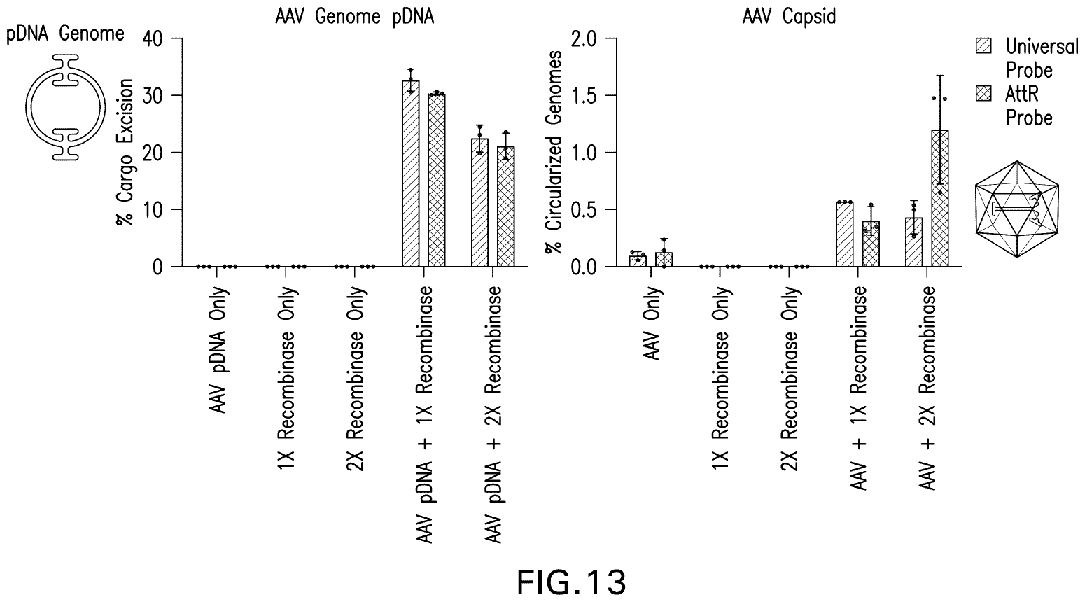

As shown in FIG. 13, the Bxb1-mediated attR scar probe provided similar percent circularization quantification compared to the universal probe.

### 7.6. Example 6: In Vitro Beacon Placement in Primary Mouse Hepatocytes and Primary Human Hepatocytes Using mRNA and AAV for Co-Delivery

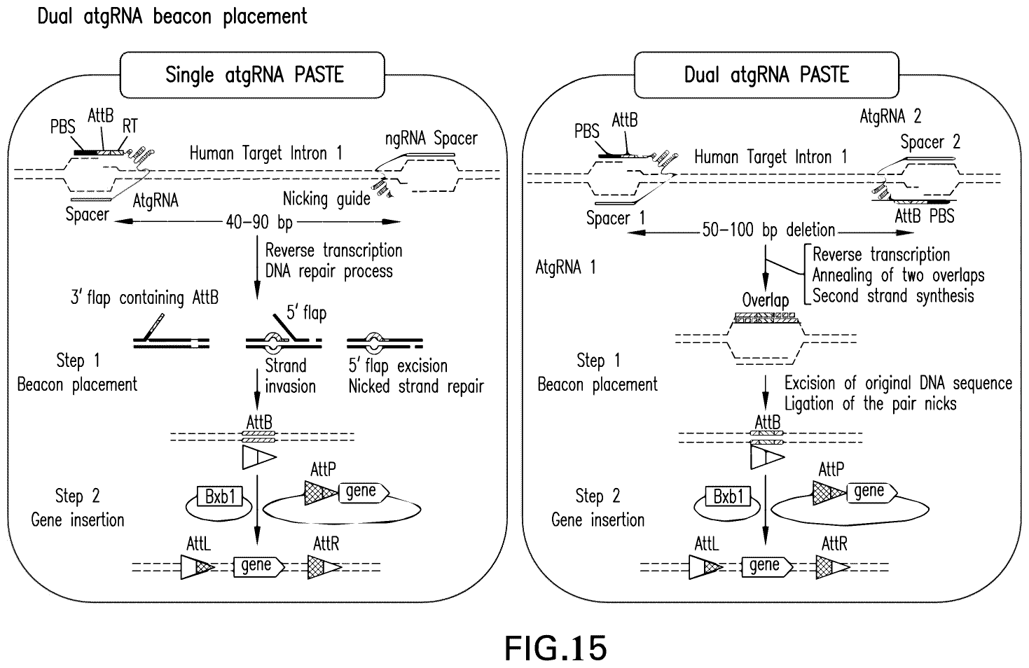

This example assessed the efficiency of in vitro beacon placement in primary human hepatocytes using mRNA delivering of a polynucleotide encoding a gene editor polynucleotide construct and AAV to deliver the first and second atgRNA. See FIG. 15 for a non-limiting example of a dual atgRNA-mediated insertion of an integration recognition site.

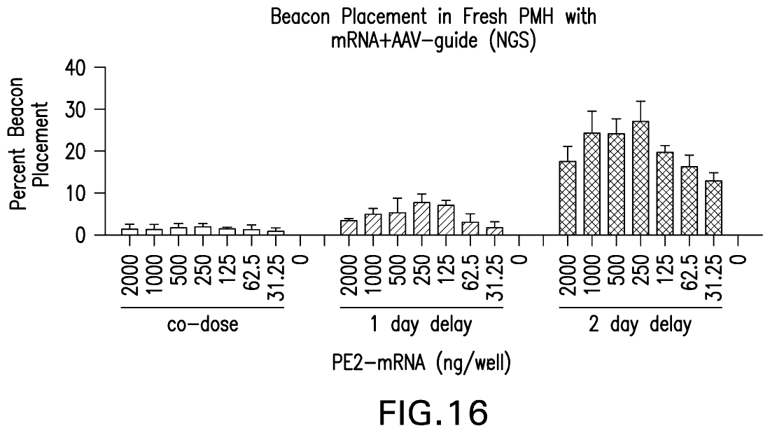

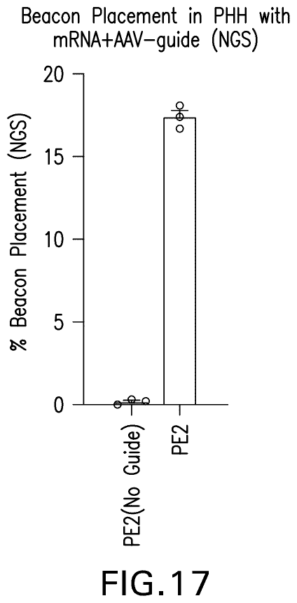

In the mouse experiments, the mRNA and AAV were delivered into the primary mouse hepatocytes (PNM) using (i) concurrent delivery (“co-dose”), (ii) AAV delivery followed by a “1-day delay” before delivery of the mRNA, or (iii) AAV delivery followed by a “2-day delay” before delivery of the mRNA. Beacon placement was then assessed using next-generation sequencing of DNA isolated from cells subjected to the delivery conditions mentioned above. The mRNA encoding the gene editor polynucleotide construct was delivered in various amounts per well: 2000 ng, 1000 ng, 500 ng, 250 ng, 125 ng, 62.5 ng, and 31.25 ng. AAV encoding the first and second atgRNA (see Table 12). The primary mouse hepatocyte data is shown in FIG. 16 and the human primary hepatocyte data is shown in FIG. 17.

TABLE 12
atgRNAs

| SEQ | | | |
|---|---|---|---|
| ID | | | |
| NO: | Target | Name | Sequence |
| 559 | Mouse | AAV- | GACGCGTTTTACCCGGAGCAGTTTAAGA |
| | Nolc1 | mNolc1-F | GCTATGCTGGAAACAGCATAGCAAGTTT |
| | | (AAVG023) | AAATAAGGCTAGTCCGTTATCAACTTGA |
| | | | AAAAGTGGCACCGAGTCGGTGCACGACG |
| | | | GAGACCGCCGTCGTCGACAAGCCTCCGG |
| | | | GTAAAACG |
| 560 | Mouse | AAV- | ACAAGGGGATAAAGGTCGCTGTTTAAGA |
| | Nolc1 | mNolc1-R | GCTATGCTGGAAACAGCATAGCAAGTTT |
| | | | AAATAAGGCTAGTCCGTTATCAACTTGA |
| | | | AAAAGTGGCACCGAGTCGGTGCACGACG |
| | | | GCGGTCTCCGTCGTCAGGATCATGACCT |
| | | | TTATCCCC |
| 561 | Human | AAV-hF9-F | CTTGTATGCCCCGAGAAGTGGTTTTAGA |
| | Factor | (AAVG048) | GCTAGAAATAGCAAGTTAAAATAAGGCT |
| | IX | | AGTCCGTTATCAACTTGAAAAAGTGGCA |
| | | | CCGAGTCGGTGCACGACGGAGACCGCCG |
| | | | TCGTCGACAAGCCTTCTCGGGGCATA |
| 562 | Human | AAV-hF9-R | TATATATACTTGCTAGGGCTGTTTTAGA |
| | Factor | (AAVG048) | GCTAGAAATAGCAAGTTAAAATAAGGCT |
| | IX | | AGTCCGTTATCAACTTGAAAAAGTGGCA |
| | | | CCGAGTCGGTGCACGACGGCGGTCTCCG |
| | | | TCGTCAGGATCATCCTAGCAAGTATA |

As shown in FIG. 16, in primary mouse hepatocytes (PMH) delivering the first atgRNAs (SEQ ID NO: 543) and the second atgRNA (SEQ ID NO: 544) using AAV at day 0 and then delivering the mRNA encoding the gene editing polynucleotide construct at day 2 (“2 day delay”) resulted in greater than 10% beacon placement for each amount of mRNA tested. Surprisingly, a 2 day delay resulted in greater beacon placement than either no delay (“co-dose) or a 1 day delay.

As shown in FIG. 17, in primary human hepatocytes (PHH), using AAV to deliver the first atgRNA (SEQ ID NO: 545) and the second atgRNA (SEQ ID NO: 546) and mRNA to deliver the gene editing polynucleotide construct resulted in about 17% beacon placement.

Taken together, this data showed robust ex vivo beacon placement in primary mouse and primary human hepatocytes.

### 7.7. Example 7: In Vivo Beacon Placement with mRNA+AAV Guide

In vivo beacon placement in mice was assessed using AAV to deliver the first and second atgRNAs and mRNA to delivery the gene editing polynucleotide construct.

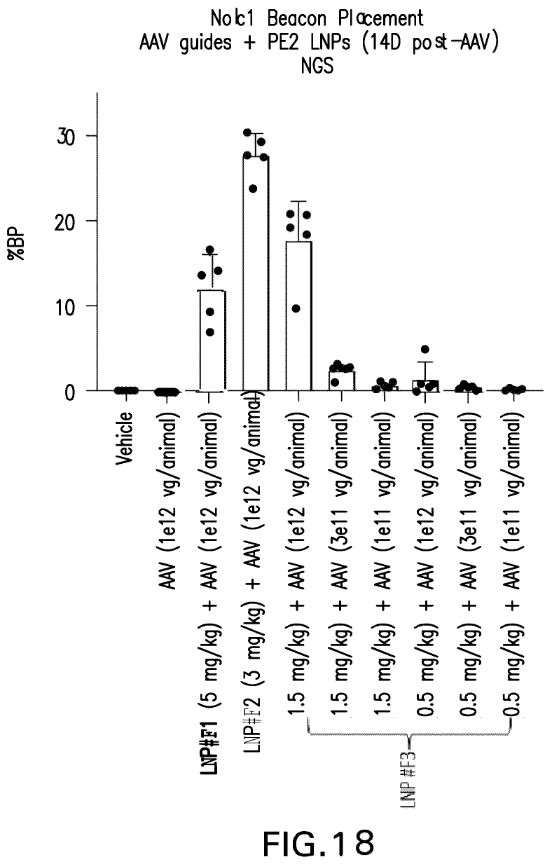

In these experiments, mice were administered AAV containing the first atgRNA (SEQ ID NO: 543; Table 12) and the second atgRNA (SEQ ID NO: 544) targeting the Nolc1 locus at 3E11 to 1E12 vector genomes (vg) per animal two 2 weeks prior to administration of the mRNA containing the gene editing polynucleotide construct (see FIG. 18). mRNA was delivered using various LNP formulations (e.g., LP01 (LNP #F1), ALC-0315 (i.e., LNP #F2), and cKK-E12 (i.e., LNP #F3)) at concentrations ranging from 5 mg/kg to 0.5 mg/kg via intravenous injection (see FIG. 18). After delivery of the mRNA, liver tissue was harvested, genomic DNA was isolated, and beacon efficiency was assessed by NGS. As shown in FIG. 18, three conditions resulted in vivo beacon placement efficiency greater than 10%.

Taken together, this data provided proof-of-concept for successful in vivo beacon placement using AAV to deliver the first and second atgRNA and LNPs to deliver the mRNA encoding the gene editor polynucleotide construct.

### 7.8. Example 8: In Vivo Integration in Mice Using AAV to Deliver the Template Polynucleotide and Adenovirus to Deliver BxB1

In vivo integration efficiency in AttP mice was assessed using adenovirus to deliver an integrase (e.g., Bxb1) and an AAV to deliver the template polynucleotide.

For these experiments, the adenovirus (i.e., adenovirus containing polynucleotide encoding the integrase) and the AAV (i.e., AAV containing the template polynucleotide and an attB site) were administered to mice containing dual AttP sites integrated in to the Rosa26 locus (B6.RosaBxb-GT/GA; female, Strain #036152). The Rosa26 locus included a first AttP site comprising a GT dinucleotide and a second AttP site comprising a GA dinucleotide. The AAV was a scAAV8 containing a vector having a template polynucleotide and a 38 bp GT AttB site. The Adenovirus was an adenovirus-type 5 (Ad5) containing a polynucleotide encoding Bxb1 (“Bxb1 AdV”) (SEQ TD NO: 563; Table 14). Mice were administered the adenovirus and AAV according to the experimental details in Table 13.

TABLE 13
Experimental Details for assessment of in vivo integration efficiency

| | | | Cargo AAV | | | | |
|---|---|---|---|---|---|---|---|
| | | Bxb1 AdV dose | Dose | | Volume | Conc. | Time |
| Group | n | (vg/animal) | (vg/animal) | Route | (ul) | (vg/ml) | points |
| 1 | 1F, 2M | vehicle | | IV | 100 | | Liver |
| 2 | 5 | 3E10 | 1E12 | IV | 100 | 3E11 + 1E13 | punches |
| 3 | 5 | 1E11 | 1E12 | IV | 100 | 1E12 + 1E13 | at 10 |
| | | | | | | | days post- |
| | | | | | | | dose |

TABLE 14
Adenovirus Vector

| Vectors | Sequence |
|---|---|
| Bxb1 AdV | TCGCGCGTTTCGGTGATGACGGTGAAAACCTCTGACACATGCAGCTCCCGGAGACG |
| (SEQ ID | GTCACAGCTTGTCTGTAAGCGGATGCCGGGAGCAGACAAGCCCGTCAGGGCGCGT |
| NO: 563) | CAGCGGGTGTTGGCGGGTGTCGGGGCTGGCTTAACTATGCGGCATCAGAGCAGATT |
| | GTACTGAGAGTGCACCATATGCGGTGTGAAATACCGCACAGATGCGTAAGGAGAA |
| | AATACCGCATCAGGCGCCATTCGCCATTCAGGCTGCGCAACTGTTGGGAAGGGCGA |
| | TCGGTGCGGGCCTCTTCGCTATTACGCCAGCTGGCGAAAGGGGGATGTGCTGCAAG |
| | GCGATTAAGTTGGGTAACGCCAGGGTTTTCCCAGTCACGACGTTGTAAAACGACGG |
| | CCAGTGAATTCGAGCTCTCGCTATTACTTGGCCACTCCCTCTCTGCGCGCTCGCTCG |
| | CTCACTGAGGCCGGGCGACCAAAGGTCGCCCGACGCCCGGGCTTTGCCCGGGCGG |
| | CCTCAGTGAGCGAGCGAGCGCGCAGAGAGGGAGTGGCCAACTCCATCACTAGGGG |
| | TTCCTCACTGCCCGCAGATCTACTAGTGGCTTGTCGACGACGGCGGTCTCCGTCGTC |
| | AGGATCATTAGGTCAGTGAAGAGAAGAACAAAAAGCAGCATATTACAGTTAGTTG |
| | TCTTCATCAATCTTTAAATATGTTGTGTGGTTTTTCTCTCCCTGTTTCCACAGTTATG |
| | GGCAACAGCTTCAGCACCAGCGCCTTCGGCCCTGTGGCCTTTTCTCTGGGCCTCCTG |
| | CTCGTGCTGCCTGCCGCTTTTCCAGCTCCTGTGTTCACCCTGGAAGATTTCGTGGGA |
| | GATTGGCGGCAGACCGCCGGCTACAACCTGGACCAAGTGCTGGAACAGGGCGGAG |
| | TGTCCAGCCTGTTTCAGAACCTGGGCGTCTCCGTGACCCCTATCCAGCGGATCGTGC |
| | TGAGCGGCGAGAACGGCCTGAAAATCGACATCCATGTGATTATCCCCTACGAGGGC |
| | CTGAGCGGAGATCAGATGGGCCAGATCGAGAAAATCTTCAAGGTGGTGTACCCCG |
| | TCGACGACCACCACTTCAAGGTGATCCTGCACTACGGCACCCTGGTGATCGACGGC |
| | GTTACCCCTAACATGATCGACTACTTCGGCAGACCCTATGAGGGAATTGCCGTGTT |
| | CGACGGCAAGAAAATCACCGTGACCGGCACACTGTGGAACGGCAACAAGATCATC |
| | GATGAGCGCCTGATCAACCCAGACGGCAGCCTGCTGTTCAGAGTGACAATCAATGG |
| | CGTGACAGGCTGGAGACTTTGTGAAAGAATCCTGGCCGGTTCTGGCGAGGGCAGA |
| | GGATCTCTGCTGACATGCGGCGATGTGGAAGAGAATCCTGGACCTGCTATGAAAAT |
| | CGAGTGCAGAATTACAGGCACACTGAACGGAGTTGAATTCGAGCTGGTCGGCGGA |
| | GGCGAGGGCACACCTGAGCAGGGCAGAATGACCAACAAGATGAAAAGCACCAAG |
| | GGCGCCCTGACCTTTTCTCCTTACCTGCTGAGCCACGTGATGGGCTATGGCTTCTAC |
| | CACTTCGGCACCTACCCCAGCGGCTATGAAAACCCCTTCCTGCATGCTATCAACAA |
| | CGGAGGCTACACCAATACCAGAATCGAGAAGTACGAGGACGGCGGCGTGCTGCAC |
| | GTGTCCTTCAGCTACAGATACGAGGCCGGCAGAGTGATCGGCGACTTCAAGGTGGT |
| | GGGCACAGGATTTCCAGAAGATAGCGTGATCTTCACCGACAAGATCATCCGGAGC |
| | AACGCCACCGTGGAACACCTGCACCCCATGGGCGATAATGTGCTGGTGGGCTCCTT |
| | TGCTAGAACATTCTCCCTGCGGGACGGCGGATACTACAGCTTCGTGGTCGACAGCC |
| | ACATGCACTTCAAGTCTGCCATCCACCCTTCTATCCTGCAGAACGGCGGACCTATGT |
| | TCGCCTTCCGGCGGGTGGAGGAACTCCACAGCAACACCGAGCTGGGCATCGTGGA |
| | ATACCAGCACGCCTTTAAGACCCCTATCGCCTTCGCCAGAAGCAGAGCCAGGTGAG |
| | AGTTTAAACCCGCTGATCAGCCTCGACTGTGCCTTCTAGTTGCCAGCCATCTGTTGT |
| | TTGCCCCTCCCCCGTGCCTTCCTTGACCCTGGAAGGTGCCACTCCCACTGTCCTTTC |
| | CTAATAAAATGAGAAAATTGCATCGCATTGTCTGAGTAGGTGTCATTCTATTCTGG |
| | GGGGGGGGTGGGGCAGGACAGCAAGGGGGAGGATTGGGAAGACAATAGCAGGC |
| | ATGCTGGGGATGCGGTGGGCTCTATGGACTAGTAGATCTCACTGCCCGCCCACTCC |
| | CTCTCTGCGCGCTCGCTCGCTCACTGAGGCCGGGCGACCAAAGGTCGCCCGACGCC |
| | CGGGCTTTGCCCGGGCGGCCTCAGTGAGCGAGCGAGCGCGCAGAGAGGGATGCAT |
| | TAATGGGATCCTCTAGAGTCGACCTGCAGGCATGCAAGCTTGGCGTAATCATGGTC |
| | ATAGCTGTTTCCTGTGTGAAATTGTTATCCGCTCACAATTCCACACAACATACGAGC |
| | CGGAAGCATAAAGTGTAAAGCCTGGGGTGCCTAATGAGTGAGCTAACTCACATTA |
| | ATTGCGTTGCGCTCACTGCCCGCTTTCCAGTCGGGAAACCTGTCGTGCCAGCTGCAT |
| | TAATGAATCGGCCAACGCGCGGGGAGAGGCGGTTTGCGTATTGGGCGCTCTTCCGC |
| | TTCCTCGCTCACTGACTCGCTGCGCTCGGTCGTTCGGCTGCGGCGAGCGGTATCAGC |
| | TCACTCAAAGGCGGTAATACGGTTATCCACAGAATCAGGGGATAACGCAGGAAAG |
| | AACATGTGAGCAAAAGGCCAGCAAAAGGCCAGGAACCGTAAAAAGGCCGCGTTGC |
| | TGGCGTTTTTCCATAGGCTCCGCCCCCCTGACGAGCATCACAAAAATCGACGCTCA |
| | AGTCAGAGGTGGCGAAACCCGACAGGACTATAAAGATACCAGGCGTTTCCCCCTG |
| | GAAGCTCCCTCGTGCGCTCTCCTGTTCCGACCCTGCCGCTTACCGGATACCTGTCCG |
| | CCTTTCTCCCTTCGGGAAGCGTGGCGCTTTCTCATAGCTCACGCTGTAGGTATCTCA |
| | GTTCGGTGTAGGTCGTTCGCTCCAAGCTGGGCTGTGTGCACGAACCCCCCGTTCAG |
| | CCCGACCGCTGCGCCTTATCCGGTAACTATCGTCTTGAGTCCAACCCGGTAAGACA |
| | CGACTTATCGCCACTGGCAGCAGCCACTGGTAACAGGATTAGCAGAGCGAGGTAT |
| | GTAGGCGGTGCTACAGAGTTCTTGAAGTGGTGGCCTAACTACGGCTACACTAGAAG |
| | GACAGTATTTGGTATCTGCGCTCTGCTGAAGCCAGTTACCTTCGGAAAAAGAGTTG |
| | GTAGCTCTTGATCCGGCAAACAAACCACCGCTGGTAGCGGTGGTTTTTTTGTTTGCA |
| | AGCAGCAGATTACGCGCAGAAAAAAAGGATCTCAAGAAGATCCTTTGATCTTTTCT |
| | ACGGGGTCTGACGCTCAGTGGAACGAAAACTCACGTTAAGGGATTTTGGTCATGAG |
| | ATTATCAAAAAGGATCTTCACCTAGATCCTTTTAAATTAAAAATGAAGTTTTAAATC |
| | AATCTAAAGTATATATGAGTAAACTTGGTCTGACAGTTACCAATGCTTAATCAGTG |
| | AGGCACCTATCTCAGCGATCTGTCTATTTCGTTCATCCATAGTTGCCTGACTCCCCG |
| | TCGTGTAGATAACTACGATACGGGAGGGCTTACCATCTGGCCCCAGTGCTGCAATG |
| | ATACCGCGAGACCCACGCTCACCGGCTCCAGATTTATCAGCAATAAACCAGCCAGC |
| | CGGAAGGGCCGAGCGCAGAAGTGGTCCTGCAACTTTATCCGCCTCCATCCAGTCTA |
| | TTAATTGTTGCCGGGAAGCTAGAGTAAGTAGTTCGCCAGTTAATAGTTTGCGCAAC |
| | GTTGTTGCCATTGCTACAGGCATCGTGGTGTCACGCTCGTCGTTTGGTATGGCTTCA |
| | TTCAGCTCCGGTTCCCAACGATCAAGGCGAGTTACATGATCCCCCATGTTGTGCAA |
| | AAAAGCGGTTAGCTCCTTCGGTCCTCCGATCGTTGTCAGAAGTAAGTTGGCCGCAG |
| | TGTTATCACTCATGGTTATGGCAGCACTGCATAATTCTCTTACTGTCATGCCATCCG |
| | TAAGATGCTTTTCTGTGACTGGTGAGTACTCAACCAAGTCATTCTGAGAATAGTGT |
| | ATGCGGCGACCGAGTTGCTCTTGCCCGGCGTCAATACGGGATAATACCGCGCCACA |
| | TAGCAGAACTTTAAAAGTGCTCATCATTGGAAAACGTTCTTCGGGGCGAAAACTCT |
| | CAAGGATCTTACCGCTGTTGAGATCCAGTTCGATGTAACCCACTCGTGCACCCAAC |
| | TGATCTTCAGCATCTTTTACTTTCACCAGCGTTTCTGGGTGAGCAAAAACAGGAAG |
| | GCAAAATGCCGCAAAAAAGGGAATAAGGGCGACACGGAAATGTTGAATACTCATA |
| | CTCTTCCTTTTTCAATATTATTGAAGCATTTATCAGGGTTATTGTCTCATGAGCGGA |
| | TACATATTTGAATGTATTTAGAAAAATAAACAAATAGGGGTTCCGCGCACATTTCC |
| | CCGAAAAGTGCCACCTGACGTCTAAGAAACCATTATTATCATGACATTAACCTATA |
| | AAAATAGGCGTATCACGAGGCCCTTTCGTC |

Ten days after administration of the AdV and AAV viruses, liver punches were collected and genomic DNA was isolated. ddPCR of the genomic DNA was used to assess integration efficiency.

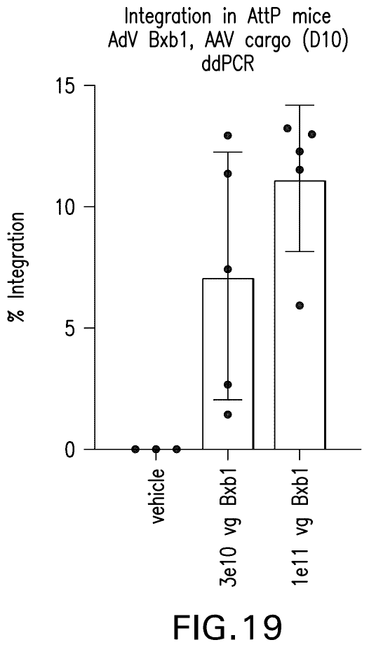

As shown in FIG. 19, administering the AAV and AdV resulted in in vivo integration of the donor polynucleotide template into the AttP mice. In particular, 3E10 vg/animal BxB1 AdV resulted in about 7% in vivo integration efficiency (see FIG. 19). Administering increased amounts of BxB1 AdV, 1E11 vg/animal, resulted in higher integration efficiency, about 11%, in AttP mice than with lower amount of 3E10 vg/animal (see FIG. 19).

Overall, this data establishes proof-of-concept for in vivo integration using an adenovirus to deliver and drive expression of Bxb1 and an AAV to deliver the template polynucleotide to be integrated into a mammalian genome, in this case, the mouse genome.

### 7.9. Example 9: In Vivo Beacon Placement in Neonatal Mice Using Split LNP

In vivo beacon placement was assessed in neonatal mice following administration of a single dose of a mixture of two LNPs. The first LNP contained mRNA encoding a prime editing system and a first synthetic atgRNA (atgRNA1). The mRNA and atgRNA1 were included at 1:1 ratio in the first LNP. The second LNP contained mRNA encoding a prime editing system and a second synthetic atgRNA (atgRNA2). The mRNA and atgRNA2 were included at a 1:1 ratio in the second LNP. Each of the first and second atgRNAs targeted the mouse Nolc1 locus and each encoded a portion of an integration recognition site (a “beacon”). AtgRNA1 and atgRNA2 together included a 6 bp overlap. The first and second LNPs were combined 1:1 as mixture prior to administration. The first atgRNA and second atgRNA are provide in Table 15, where the atgRNA include one or more 2′O-methyl modifications and one or more phosphorothioate linkages.

TABLE 15
atgRNAs

| SEQ | | | |
|---|---|---|---|
| ID | | | |
| NO: | Target | Name | Sequence |
| 564 | Mouse | mNolc1-F | mG*mA*mC*rGrCrGrUrUrUr |
| | Nolc1 | (synthetic | UrArCrCrCrGrGrArGrCrAr |
| | | guide, | GrUrUrUrUrArGrAmGmCmUm |
| | | 6 bp | AmGmAmAmAmUmAmGmCrArAr |
| | | overlap) | GrUrUrArArArArUrArArGr |
| | | | GrCrUrArGrUrCrCrGrUrUr |
| | | | ArUrCrAmAmCmUmUmGmAmAm |
| | | | AmAmAmGmUmGmGmCmAmCmCm |
| | | | GmAmGmUmCmGmGmUmGmCrAr |
| | | | GrArCrCrGrCrCrGrUrCrGr |
| | | | UrCrGrArCrArArGrCrCrUr |
| | | | CrCrGrGrGrUrArArA*mA* |
| | | | mC*mG |
| 565 | Mouse | mNolc1-R | mA*mC*mA*rArGrGrGrGrAr |
| | Nolc1 | (synthetic | UrArArArGrGrUrCrGrCrUr |
| | | guide, | GrUrUrUrUrArGrAmGmCmUm |
| | | 6 bp | AmGmAmAmAmUmAmGmCrArAr |
| | | overlap) | GrUrUrArArArArUrArArGr |
| | | | GrCrUrArGrUrCrCrGrUrUr |
| | | | ArUrCrAmAmCmUmUmGmAmAm |
| | | | AmAmAmGmUmGmGmCmAmCmCm |
| | | | GmAmGmUmCmGmGmUmGmCrCr |
| | | | GrGrUrCrUrCrCrGrUrCrGr |
| | | | UrCrArGrGrArUrCrArUrGr |
| | | | ArCrCrUrUrUrArUrC*mC* |
| | | | mC*mC |

The LNP mixture was administered to the neonatal mice (2-5 day old CD-1 mice) according to the experimental details in Table 16.

TABLE 16
Experimental details for in vivo beacon
placement in neonatal mice.

| | | | Dose | | Volume | Conc. | Time |
|---|---|---|---|---|---|---|---|
| Group | n | Treatment | (mg/kg) | Route | (ml/kg) | (mg/ml) | points |
| 1 | 5 | vehicle | | IV | 5 | | Whole |
| 2 | 3 | LNP | 1 | IV | 5 | 0.2 | liver on |
| 3 | 4 | LNP | 3 | IV | 5 | 0.6 | day 8 |
| | | | | | | | post-dose |
| | | | | | | | (168 |
| | | | | | | | hours) |
| 4 | 5 | vehicle | | IV | 5 | | Liver |
| 5 | 5 | LNP | 1 | IV | 5 | 0.2 | punches |
| 6 | 5 | LNP | 3 | IV | 5 | 0.6 | (one 8 mm |
| | | | | | | | punch |
| | | | | | | | from each |
| | | | | | | | lobe) at 6 |
| | | | | | | | weeks |
| | | | | | | | post-dose |

Eight days after administration of the LNP mixture in vivo beacon placement was assessed. In particular, at day 8 post administration, liver samples (either whole liver for groups 1-3 or liver punches from each lobe for groups 4-6 (see Table 13)) were collected and genomic DNA was isolated. Beacon placement was detected using ddPCR and NGS.

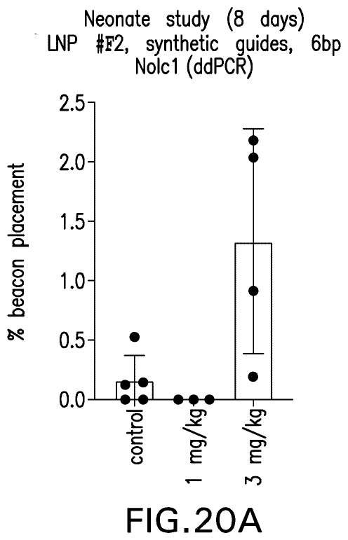

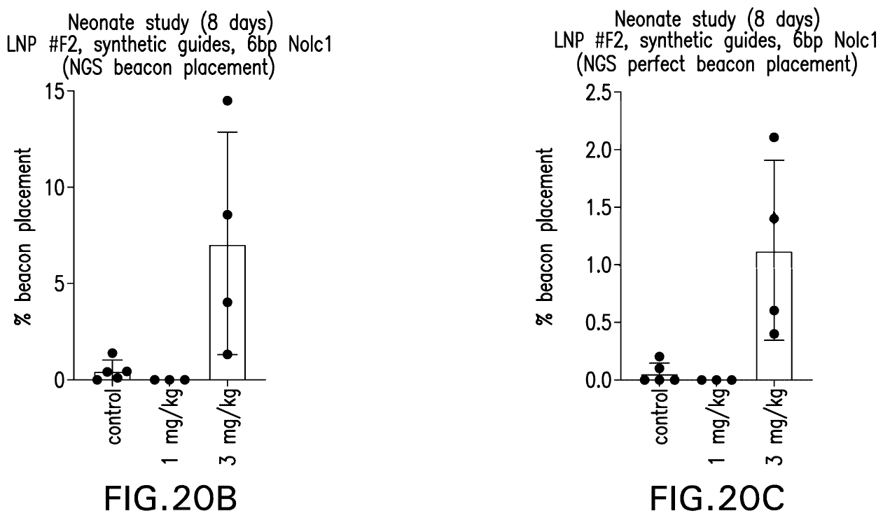

As shown in FIG. 20A, ddPCR revealed about 1% beacon placement (in Nolc1 alleles) following administration of a 3 mg/kg dose of the LNP mixture. Confirmation of beacon placement using NGS showed about 7% beacon placement (in Nolc1 alleles) following administration of a 3 mg/kg dose of the LNP mixture (see FIG. 20B). In order to determine what percentage of the integrated beacons included the expected integration recognition site (“perfect beacon”), an NGS-based assay was used to make this assessment. As shown in FIG. 20C, about 1% of the integrated beacons contained the expected integration recognition site.

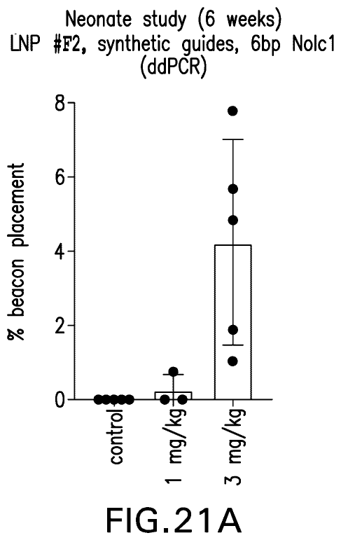

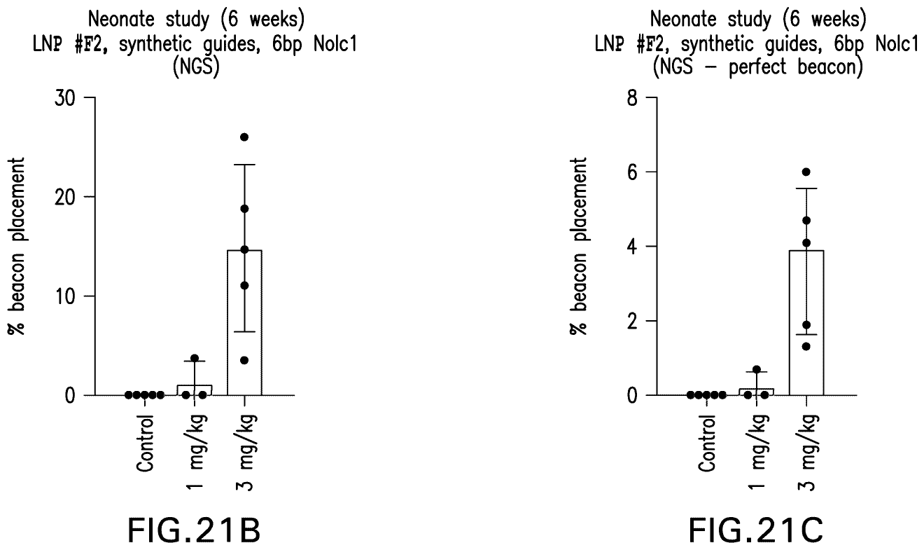

Neonates were also assessed at six weeks after administration of the LNP mixture. Beacon placement was detected using ddPCR and NGS. As shown in FIG. 21A., at six weeks post administration, ddPCR revealed about 4% beacon placement (in Nolc1 alleles) for a 3 mg/kg dose of the LNP mixture. Confirmation of beacon placement using NGS showed about 15% beacon placement (in Nolc1 alleles) for a 3 mg/kg dose of the LNP mixture (see FIG. 21B). Assessment of the percent of integrated beacons that included the expected integration recognition site (“perfect beacon”) revealed that about 3.5% of beacons were comprised of perfect beacons (see FIG. 21C).

Overall, this data demonstrated successful in vivo site-specific integration of an integration recognition site. In particular, this data showed that a split LNP approach can be used for site-specifically integrating an integration recognition site in vivo in a mammalian genome, in this case neonatal mice.

### 7.10. Example 10: In Vivo Beacon Placement in Mice Using Split LNP

In vivo beacon placement was assessed in adult mice using a single dose mixture of two LNPs. The first LNP contained mRNA encoding a prime editing system and a first synthetic atgRNA (atgRNA1). The mRNA and atgRNA1 were included at different ratios (e.g., 1:0.5, 1:1, and 1:2) ratio in the first LNP. The second LNP contained mRNA encoding a prime editing system and a second synthetic atgRNA (atgRNA2). The mRNA and atgRNA2 were included at different ratios (e.g., 1:0.5, 1:1, and 1:2) ratio in the second LNP. Here, the first and second atgRNAs targeted mouse Factor IX (“mF9”) locus and each encoded a portion of an integration recognition site (“beacon”). Similar to Example 9, atgRNA1 and atgRNA2 together included a 6 bp overlap and were combined 1:1 as mixture prior to administration. The first atgRNA and second atgRNA are provide in Table 17, where the atgRNA include one or more 2′O-methyl modifications and one or more phosphorothioate linkages.

TABLE 17
atgRNAs

| SEQ | | | |
|---|---|---|---|
| ID | | | |
| NO: | Target | Name | Sequence |
| 566 | Mouse | mF9-F | mA*mG*mU*rGrArCrArGrUrGrC |
| | Factor | (synthetic | rCrArGrGrArUrCrArGrGrUrUr |
| | IX | guide, | UrUrArGrAmGmCmUmAmGmAmAmA |
| | | 6 bp | mUmAmGmCrArArGrUrUrArArAr |
| | | overlap) | ArUrArArGrGrCrUrArGrUrCrC |
| | | | rGrUrUrArUrCrAmAmCmUmUmGm |
| | | | AmAmAmAmAmGmUmGmGmCmAmCmC |
| | | | mGmAmGmUmCmGmGmUmGmCrArGr |
| | | | ArCrCrGrCrCrGrUrCrGrUrCrG |
| | | | rArCrArArGrCrCrArUrCrCrUr |
| | | | GrGrCrArCmU*mG*mU |
| 567 | Mouse | mF9-R | mG*mU*mU*rGrArCrArUrCrArU |
| | Factor | (synthetic | rGrUrCrUrGrGrArGrUrGrUrUr |
| | IX | guide, | UrUrArGrAmGmCmUmAmGmAmAmA |
| | | 6 bp | mUmAmGmCrArArGrUrUrArArAr |
| | | overlap) | ArUrArArGrGrCrUrArGrUrCrC |
| | | | rGrUrUrArUrCrAmAmCmUmUmGm |
| | | | AmAmAmAmAmGmUmGmGmCmAmCmC |
| | | | mGmAmGmUmCmGmGmUmGmCrCrGr |
| | | | GrUrCrUrCrCrGrUrCrGrUrCrA |
| | | | rGrGrArUrCrArUrCrCrArGrAr |
| | | | CrArUrGrAmU*mG*mU |

In particular, the LNP mixture was administered to female CD-1 mice 6-8 weeks old according to the experimental details in Table 18.

TABLE 18
Experimental details for in vivo beacon placement in adult mice

| | | Treatment | | | | | |
|---|---|---|---|---|---|---|---|
| | | (ratio | Dose | | Volume | Conc. | Time |
| Group | n | mRNA:atgRNA1:atgRNA2) | (mg/kg) | Route | (ml/kg) | (mg/ml) | points |
| 1 | 5 | vehicle | | IV | 5 | | Terminal: |
| 2 | 5 | 1:0.25:0.25* | 3 | IV | 5 | 0.6 | liver |
| 3 | 5 | 1:0.5:0.5** | 3 | IV | 5 | 0.6 | punches |
| 4 | 5 | 1:1:1*** | 3 | IV | 5 | 0.6 | on day 8 |

*1:0.25:0.25 = mRNA:atgRNA1 1:0.5; mRNA:atgRNA2 1:0.5; LNPs mixed 1:1
**1:0.5:0.5 = mRNA:atgRNA1 1:1; mRNA:atgRNA2 1:1; LNPs mixed 1:1
***1:1:1 = mRNA:atgRNA1 1:2; mRNA:atgRNA2 1:2; LNPs mixed 1:1

Eight days after administration of the LNP mixture in vivo beacon placement was assessed. In particular, at day 8 post administration, liver samples (i.e., liver punches of each lobe (see Table 14)) were collected and genomic DNA was isolated. Beacon placement was detected using ddPCR and NGS.

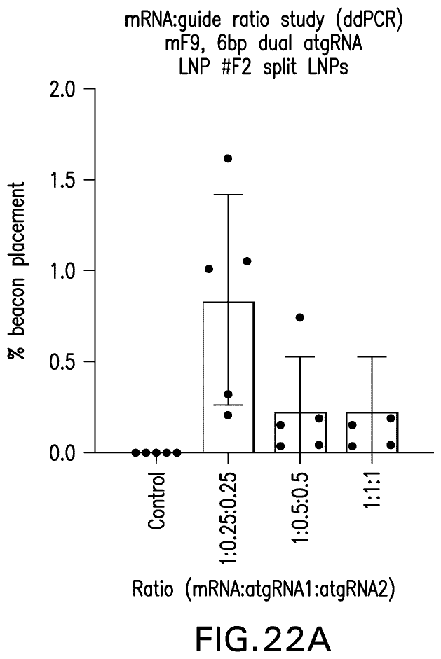

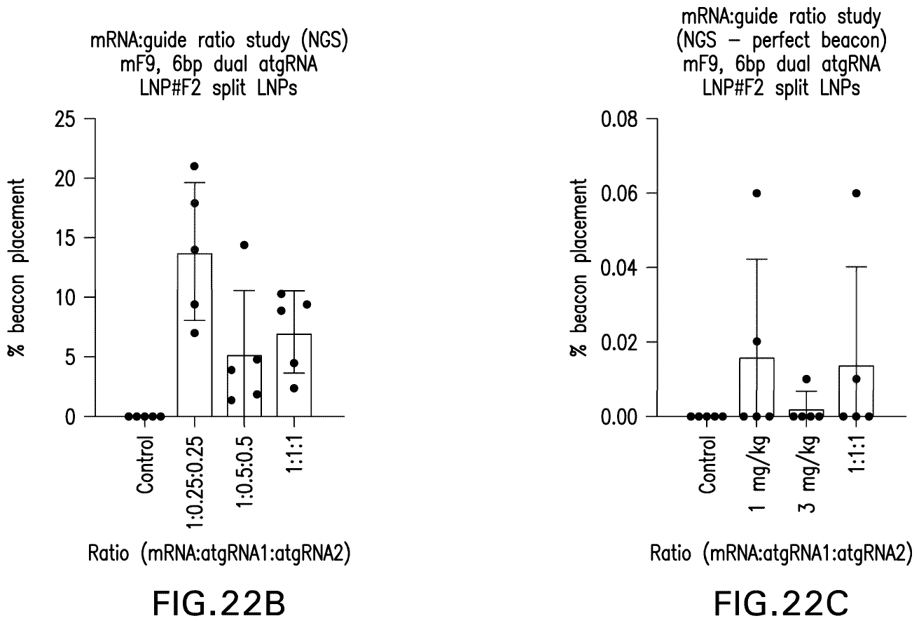

As shown in FIG. 22A, ddPCR revealed about 0.8% beacon placement (in mF9 alleles) following administration of a 1:0.25:0.25 ratio of mRNA:atgRNA1:atgRNA2. Confirmation of beacon placement using NGS showed about 14% beacon placement (in mF9 alleles) following administration of the 1:0.25:0.25 ratio of mRNA:atgRNA1:atgRNA2 (see FIG. 22B). Similar to Example 9, an NGS-based assay was used to determined what percentages of the integrated beacons included the expected integration recognition site (“perfect beacon”). As shown in FIG. 22C, about 0.02% of the beacons placed in the mF9 locus were “perfect” beacons.

Overall, this data showed successful in vivo site-specific integration of an integration recognition site in adult mice. In particular, this data showed that the ratio of mRNA to atgRNA is an important consideration in determining efficacy of in vivo site-specific integration of an integration recognition site.

### 8. EQUIVALENTS AND INCORPORATION BY REFERENCE

All references cited herein are incorporated by reference to the same extent as if each individual publication, database entry (e.g. Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

It is an object of the invention not to encompass within the invention any previously known product, process of making the product, or method of using the product such that Applicant reserves the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. 112(a)) or the EPO (Article 83 of the EPC), such that Applicant reserves the right and hereby disclose a disclaimer of any previously described product, process of making the product, or method of using the product. It may be advantageous in the practice of the invention to be in compliance with Art. 53(c) EPC and Rule 28(b) and (c) EPC. Nothing herein is to be construed as a promise. It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

While the invention has been particularly shown and described with reference to a preferred embodiment and various alternate embodiments, it is understood by persons skilled in the relevant art that various changes in form and details can be made therein without departing from the spirit and scope of the invention.

What is claimed is:

1. A method for delivering a system capable of site-specifically integrating a template polynucleotide into the genome of a cell, the method comprising:
delivering to a cell:
(a) a lipid nanoparticle (LNP) comprising:
(i) a gene editor polynucleotide; and
(b) a vector comprising:
(i) a template polynucleotide, and
(ii) at least a first attachment site-containing guide RNA (atgRNA).

2. The method of claim 1, wherein the gene editor polynucleotide is capable of localizing to a cell cytoplasm.

3. The method of claim 1, wherein the template polynucleotide is capable of localizing to a cell nucleus.

4. The method of claim 1 or 2, wherein the gene editor polynucleotide comprises: a polynucleotide sequence encoding a prime editor system.

5. The method of claim 4, wherein the prime editor system comprises a nucleotide sequence encoding a nickase and a nucleotide sequence encoding a reverse transcriptase.

6. The method of claim 5, wherein the nucleotide sequence encoding the nickase and the nucleotide sequence encoding the reverse transcriptase are positioned in the gene editor polynucleotide such that when expressed the nickase is linked to the reverse transcriptase.

7. The method of claim 6, wherein the nickase is linked to the reverse transcriptase by in-frame fusion.

8. The method of claim 6, wherein the nickase is linked to the reverse transcriptase by a linker.

9. The method of claim 8, wherein the linker is a peptide fused in-frame between the nickase and reverse transcriptase.

10. The method of any one of claims 1-9, wherein the gene editor polynucleotide further comprises:
a polynucleotide sequence encoding at least a first integrase.

11. The method of claim 10, wherein the linked nickase-reverse transcriptase are further linked to the first integrase.

12. The method of any one of claims 1-9, further comprising delivering a second vector.

13. The method of claim 12, wherein the second vector comprises a polynucleotide sequence encoding at least a first integrase.

14. The method of any one of claims 10-13, wherein the first integrase is selected from BxB1, Bcec, Sscd, Sacd, Int10, or Pa01.

15. The method of any one of claims 1-14, wherein the gene editor polynucleotide further comprises a polynucleotide sequence encoding a recombinase.

16. The method of claim 15, wherein the recombinase is FLP or Cre.

17. The method of any one of claims 1-16, wherein the first atgRNA comprises:
(i) a domain that is capable of guiding the prime editor system to a target sequence; and
(ii) a reverse transcriptase (RT) template that comprises at least a portion of an at least first integration recognition site.

18. The method of claim 14, wherein the RT template comprises the entirety of the first integration recognition site.

19. The method of any one of claim 1-15, wherein the vector further comprises a second atgRNA.

20. The method of claim 19, wherein the first atgRNA and the second atgRNA are an at least first pair of atgRNAs, wherein
the at least first pair of atgRNAs have domains that are capable of guiding the prime editor system to a target sequence;
the first atgRNA further includes a first RT template that comprises at least a portion of an at least first integration recognition site;
the second atgRNA further includes a second RT template that comprises at least a portion of the first integration recognition site, and
the first atgRNA and the second atgRNAs collectively encode the entirety of the first integration recognition site.

21. The method of any one of claims 1-18, wherein the vector further comprises a nicking gRNA.

22. The method of any one of claims 1-18, wherein the LNPs further comprises a nicking gRNA.

23. The method of any one of claims 1-21, wherein the template polynucleotide comprises at least one of the following: a gene, an expression cassette, a logic gate system, or any combination thereof.

24. The method of any one of claims 1-23, wherein the template polynucleotide comprises a second integration recognition site.

25. The method of claim 24, wherein the second integration recognition site is a cognate pair with the first integration recognition site.

26. The method of any one of claims 1-23, wherein the template polynucleotide comprises at least a third integration recognition site.

27. The method of claim 26, wherein the template polynucleotide further comprises at least a fourth integration recognition site.

28. The method of claim 26, wherein the third integration recognition site and the fourth integration recognition site are selected from attB, attB2, attP, or attP2.

29. The method of any one of claims 1-28, wherein the vector further comprises a sub-sequence that is capable of self-circularizing to form a self-circular nucleic acid.

30. The method of claim 29, wherein the sub-sequence of the vector that is capable of self-circularizing includes the template polynucleotide, whereby upon self-circularizing the self-circular nucleic acid comprises the template polynucleotide.

31. The method of any one of claims 26-30, wherein the sub-sequence is flanked by the third integration recognition site and the fourth integration recognition site.

32. The method of claim 31, wherein self-circularizing is mediated by recombination of the third integration recognition site and a fourth integration recognition site by the integrase.

33. The method of any one of claims 29-32, wherein the self-circular nucleic acid comprises one or more additional integration recognition sites that enable integration of additional nucleic acid cargo.

34. The method of any one of claims 1-33, wherein the vector is a vector selected from: an adenovirus, an AAV, a lentivirus, a HSV, an annelovirus, a retrovirus, a Doggybone™ DNA (dbDNA), a minicircle, a plasmid, a miniDNA, an exosome, a fusosome, or a nanoplasmid.

35. The method of any one of claims 1-34, wherein the LNP and the vector are concurrently delivered.

36. The method of any one of claims 1-34, wherein the LNP and the vector are delivered separately.

37. The method of claim 36, wherein the LNP and the vector are delivered at least 1 day, 2 days, 3 days, 4 days, 5 days, 6 days, 7 days, 2 weeks, 3 weeks, 4 weeks, 5 weeks, 6 weeks, 7 weeks, or 8 weeks apart.

38. The method of any one of claims 1-37, wherein the cell is in vivo.

39. A method for delivering a system capable of site-specifically integrating a template polynucleotide into the genome of a cell, the method comprising:
delivering to a cell:
(a) a lipid nanoparticle (LNP) comprising:
(i) a gene editor polynucleotide, and
(ii) a first attachment site-containing guide RNA (atgRNA); and
(b) a vector comprising:
(i) a template polynucleotide, and
(ii) a second atgRNA.

40. A method for delivering a system capable of site-specifically integrating a template polynucleotide into the genome of a cell, the method comprising:
delivering:
(a) a lipid nanoparticle (LNP) comprising:
(i) a gene editor polynucleotide,
(ii) a first attachment site-containing guide RNA (atgRNA), and
(iii) a second atgRNA; and
(b) a vector comprising:
(i) a template polynucleotide.

41. A method for delivering a system capable of site-specifically integrating a template polynucleotide into the genome of a cell, the method comprising:
delivering:
(a) a lipid nanoparticle (LNP) comprising:
(i) a gene editor polynucleotide, and
(ii) a first attachment site-containing guide RNA (atgRNA); and
(b) a vector comprising:
(i) a template polynucleotide, and
(ii) a nicking atgRNA.

42. The method of any one of claims 39-41, wherein the gene editor polynucleotide comprises:
a polynucleotide sequence encoding a prime editor system.

43. The method of claim 42, wherein the prime editor system comprises a nucleotide sequence encoding a nickase and a nucleotide sequence encoding a reverse transcriptase.

44. The method of claim 43, wherein the nucleotide sequence encoding the nickase and the nucleotide sequence encoding the reverse transcriptase are positioned in the gene editor polynucleotide such that when expressed the nickase is linked to the reverse transcriptase.

45. The method of claim 44, wherein the nickase is linked to the reverse transcriptase by in-frame fusion.

46. The method of claim 44, wherein the nickase is linked to the reverse transcriptase by a linker.

47. The method of claim 46, wherein the linker is a peptide fused in-frame between the nickase and reverse transcriptase.

48. The method of any one of claims 39-47, wherein the gene editor polynucleotide construct further comprises:
a polynucleotide sequence encoding at least a first integrase.

49. The method of claim 48, wherein the linked nickase-reverse transcriptase are further linked to the integrase.

50. The method of any one of claims 39-49, further comprising delivering a second vector.

51. The method of claim 50, wherein the second vector comprises a polynucleotide sequence encoding at least a first integrase.

52. The method of any one of claims 48-51, wherein the first integrase is selected from BxB1, Bcec, Sscd, Sacd, Int10, or Pa01.

53. The method of any one of claims 39-52, wherein the gene editor polynucleotide construct further comprises a polynucleotide sequence encoding a recombinase.

54. The method of claim 53, wherein the recombinase is FLP or Cre.

55. The method of any one of claims 41-54, wherein the first atgRNA comprises:
(i) a domain that is capable of guiding the prime editor system to a target sequence; and
(ii) a reverse transcriptase (RT) template that comprises at least a portion of an at least first integration recognition site.

56. The method of claim 55, wherein the RT template comprises the entirety of the first integration recognition site.

57. The method of any one of claims 39, 40 or 42-54, wherein the first atgRNA and the second atgRNA are an at least first pair of atgRNAs, wherein
the at least first pair of atgRNAs have domains that are capable of guiding the prime editor system to a target sequence;
the first atgRNA further includes a first RT template that comprises at least a portion of the first integration recognition site;
the second atgRNA further includes a second RT template that comprises at least a portion of the first integration recognition site, and
the first atgRNA and the second atgRNAs collectively encode the entirety of the first integration recognition site.

58. The method of any one of claims 39-57, wherein the template polynucleotide comprises at least one of the following: a gene, an expression cassette, a logic gate system, or any combination thereof.

59. The method of any one of claims 39-58, wherein the template polynucleotide comprises a second integration recognition site.

60. The method of claim 59, wherein the second integration recognition site is a cognate pair with the first integration recognition site.

61. The method of any one of claims 39-60, wherein the template polynucleotide comprises at least a third integration recognition site.

62. The method of claim 61, wherein the template polynucleotide further comprises at least a fourth integration recognition site.

63. The method of claim 62, wherein the third integration recognition site and the fourth integration recognition site are selected from attB, attB2, attP, or attP2.

64. The method of any one of claims 39-63, wherein the vector further comprises a sub-sequence that is capable of self-circularizing to form a self-circular nucleic acid.

65. The method of claim 64, wherein the sub-sequence of the vector that is capable of self-circularizing includes the template polynucleotide, whereby upon self-circularizing the self-circular nucleic acid comprises the template polynucleotide.

66. The method of claim 64 or 65, wherein the sub-sequence is flanked by the third integration recognition site and the fourth integration recognition site.

67. The method of claim 66, wherein self-circularizing is mediated by recombination of the third integration recognition site and the fourth integration recognition site by the integrase.

68. The method of any one of claims 65-67, wherein the self-circular nucleic acid comprises one or more additional integration recognition sites that enable integration of additional nucleic acid cargo.

69. The method of any one of claims 39-68, wherein the vector is a vector selected from:
an adenovirus, an AAV, a lentivirus, a HSV, an annelovirus, a retrovirus, a Doggybone™ DNA (dbDNA), a minicircle, a plasmid, a miniDNA, a exosome, a fusosome, or a nanoplasmid.

70. The method of any one of claims 39-69, wherein the LNP and the vector are concurrently delivered.

71. The method of any one of claims 39-69, wherein the LNP and the vector are delivered separately.

72. The method of claim 71, wherein the LNP and the vector are delivered at least 1 day, 2 days, 3 days, 4 days, 5 days, 6 days, 7 days, 2 weeks, 3 weeks, 4 weeks, 5 weeks, 6 weeks, 7 weeks, or 8 weeks apart.

73. The method of any one of claims 39-72, wherein the cell is in vivo.

74. A method of co-delivering a system capable of site-specifically integrating at least a first integration recognition site into the genome of a cell, the method comprising:
co-delivering to a cell:
(a) a first lipid nanoparticle (LNP) comprising:
(i) a first gene editor polynucleotide, and
(ii) a first attachment site-containing guide RNA (atgRNA); and
(b) a second lipid nanoparticle (LNP) comprising:
(i) a second gene editor polynucleotide, and
(ii) a second attachment site-containing guide RNA (atgRNA),
wherein the first atgRNA and the second atgRNA are an at least first pair of atgRNAs.

75. The method of claim 74, further comprising mixing the first LNP and the second LNP prior to co-delivering to the cell.

76. The method of claim 75, wherein the first LNP and the second LNP are mixed at a ratio of 1:0.25, 1:0.5, 1:0.75, 1:1, 0.75:1, 0.5:1, or 0.25:1.

77. The method of any one of claims 74-76, wherein the first gene editor polynucleotide construct, the second gene editor polynucleotide construct, or both comprise:
a polynucleotide sequence encoding a prime editor system.

78. The method of claim 77, wherein the prime editor system comprises a nucleotide sequence encoding a nickase and a nucleotide sequence encoding a reverse transcriptase.

79. The method of claim 78, wherein the nucleotide sequence encoding the nickase and the nucleotide sequence encoding the reverse transcriptase are positioned in the gene editor polynucleotide such that when expressed the nickase is linked to the reverse transcriptase.

80. The method of claim 79, wherein the nickase is linked to the reverse transcriptase by in-frame fusion.

81. The method of claim 79, wherein the nickase is linked to the reverse transcriptase by a linker.

82. The method of claim 81, wherein the linker is a peptide fused in-frame between the nickase and reverse transcriptase.

83. The method of any one of claims 74-82, wherein the first gene editor polynucleotide, construct, the second gene editor polynucleotide construct, or both, further comprise:
a polynucleotide sequence encoding an integrase.

84. The method of claim 83, wherein the linked nickase-reverse transcriptase are further linked to the integrase.

85. The method of any one of claims 74-84, wherein the first gene editor polynucleotide, the second gene editor polynucleotide, or both, further comprise:
a polynucleotide sequence encoding a recombinase.

86. The method of claim 85, wherein the linked nickase-reverse transcriptase are further linked to the recombinase.

87. The method of any one of claims 74-86, wherein the first gene editor polynucleotide and the second gene editor polynucleotide are the same.

88. The method of any one of claims 74-87, wherein the first gene editor polynucleotide is mRNA, the second gene editor polynucleotide is mRNA, or both the first and second gene editor polynucleotides are mRNA.

89. The method of claim 88, wherein the first LNP comprises a ratio of mRNA to atgRNA of 1:0.25, 1:0.5, 1:0.75, 1:1, 0.75:1, 0.5:1, or 0.25:1.

90. The method of claim 88 or 89, wherein the second LNP comprises a ratio of mRNA to atgRNA of 1:0.25, 1:0.5, 1:0.75, 1:1, 0.75:1, 0.5:1, or 0.25:1.

91. The method of any one of claims 74-82, further comprising delivering an integrase.

92. The method of claim 91, wherein delivering the integrase comprises co-delivering the integrase with (a) and (b).

93. The method of claim 91 or 92, wherein the method comprises delivering a polynucleotide sequence encoding the integrase.

94. The method of claim 93, wherein the polynucleotide sequence is encoded in a first vector.

95. The method of claim 94, wherein the first vector is a vector selected from: an adenovirus, an AAV, a lentivirus, a HSV, an annelovirus, a retrovirus, a Doggybone™ DNA (dbDNA), a minicircle, a plasmid, a miniDNA, an exosome, a fusosome, or a nanoplasmid.

96. The method of claim 93, wherein the first vector further comprises a template polynucleotide and a sequence that is an integration cognate with the first integration recognition site.

97. The method of any one of claims 74-96, further comprising delivering a recombinase.

98. The method of claim 97, wherein delivering the recombinase comprises co-delivering the recombinase with (a) and (b).

99. The method of claim 97 or 98, wherein the method comprises delivering a polynucleotide sequence encoding the recombinase.

100. The method of claim 99, wherein the polynucleotide sequence is encoded in the first vector.

101. The method of any one of claims 74-100, further comprising delivering a second vector.

102. The method of claim 101, wherein the second vector comprises a template polynucleotide and a sequence that is an integration cognate with the first integration recognition site.

103. The method of claim 101, wherein the second vector is a vector selected from: an adenovirus, an AAV, a lentivirus, an HSV, an annelovirus, a retrovirus, Doggybone™ DNA (dbDNA), a minicircle, a plasmid, a miniDNA, an exosome, a fusosome, or a nanoplasmid.

104. The method of any one of claims 96-103, wherein the template polynucleotide comprises at least one of the following: a gene, an expression cassette, a logic gate system, or any combination thereof.

105. The method of any one of claims 96-104, wherein the template polynucleotide comprises a second integration recognition site.

106. The method of claim 105, wherein the second integration recognition site is a cognate pair with the first integration recognition site.

107. The method of any one of claims 96-106, wherein the template polynucleotide comprises at least a third integration recognition site.

108. The method of claim 107, wherein the template polynucleotide further comprises at least a fourth integration recognition site.

109. The method of claim 108, wherein the third integration recognition site and the fourth integration recognition site are selected from attB, attB2, attP, or attP2.

110. The method of any one of claims 96-109, wherein the vector further comprises a sub-sequence that is capable of self-circularizing to form a self-circular nucleic acid.

111. The method of claim 110, wherein the sub-sequence of the vector that is capable of self-circularizing includes the template polynucleotide, whereby upon self-circularizing the self-circular nucleic acid comprises the template polynucleotide.

112. The method of claim 110 or 111, wherein the sub-sequence is flanked by the third integration recognition site and the fourth integration recognition site.

113. The method of claim 112, wherein self-circularizing is mediated by recombination of the third integration recognition site and a fourth integration recognition site by the integrase.

114. The method of any one of claims 110-113, wherein the self-circular nucleic acid comprises one or more additional integration recognition sites that enable integration of additional nucleic acid cargo.

115. The method of any one of claims 74-114, wherein
the at least first pair of atgRNAs have domains that are capable of guiding the prime editor system to a target sequence,
the first atgRNA further includes a first RT template that comprises at least a portion of a first integration recognition site;
the second atgRNA further includes a second RT template that comprises at least a portion of the first integration recognition site, and
the first atgRNA and the second atgRNAs collectively encode the entirety of the first integration recognition site.

116. The method of claim 115, wherein the first integration site is an AttB sequence, a FRT sequence, or a VOX sequence.

117. The method of any one of claims 74-116, wherein the first atgRNA, the second atgRNA or both are synthetic.

118. The method of any one of claims 91-117, wherein the integrase is selected from BxB1, Bcec, Sscd, Sacd, Int10, or Pa01.

119. The method of any one of claims 74-118, wherein the cell is in vivo.

120. A system capable of site-specifically integrating a template polynucleotide into the genome of a cell, the system comprising:
(a) a lipid nanoparticle (LNP) comprising:
(i) a gene editor polynucleotide construct; and
(b) a vector comprising:
(i) a template polynucleotide, and
(ii) at least a first attachment site-containing guide RNA (atgRNA).

121. The system of claim 120, wherein the gene editor polynucleotide construct comprises a polynucleotide sequence encoding a prime editor system.

122. The system of claim 121, wherein the prime editor system comprises a nucleotide sequence encoding a nickase and a nucleotide sequence encoding a reverse transcriptase.

123. The system of claim 122, wherein the nucleotide sequence encoding the nickase and the nucleotide sequence encoding the reverse transcriptase are positioned in the gene editor polynucleotide such that when expressed the nickase is linked to the reverse transcriptase.

124. The system of claim 123, wherein the nickase is linked to the reverse transcriptase by in-frame fusion.

125. The system of claim 123, wherein the nickase is linked to the reverse transcriptase by a linker.

126. The system of claim 125, wherein the linker is a peptide fused in-frame between the nickase and reverse transcriptase.

127. The system of any one of claims 120-126, wherein the gene editor polynucleotide construct further comprises:
a polynucleotide sequence encoding at least a first integrase.

128. The system of claim 127, wherein the linked nickase-reverse transcriptase are further linked to the first integrase.

129. The system of any one of claims 120-126, further comprising a second vector.

130. The system of claim 129, wherein the second vector comprises a polynucleotide sequence encoding at least a first integrase.

131. The system of any one of claims 127-130, wherein the first integrase is selected from BxB1, Bcec, Sscd, Sacd, Int10, or Pa01.

132. The system of any one of claims 120-131, wherein the gene editor polynucleotide construct further comprises a polynucleotide sequence encoding a recombinase.

133. The system of claim 132, wherein the recombinase is FLP or Cre.

134. The system of any one of claims 120-133, wherein the first atgRNA comprises:
(i) a domain that is capable of guiding the prime editor system to a target sequence; and
(ii) a reverse transcriptase (RT) template that comprises at least a portion of an at least first integration recognition site.

135. The system of claim 134, wherein the RT template comprises the entirety of the first integration recognition site.

136. The system of any one of claim 120-133, wherein the vector further comprises a second atgRNA.

137. The system of claim 136, wherein the first atgRNA and the second atgRNA are an at least first pair of atgRNAs, wherein
the at least first pair of atgRNAs have domains that are capable of guiding the prime editor system to a target sequence;
the first atgRNA further includes a first RT template that comprises at least a portion of the first integration recognition site;
the second atgRNA further includes a second RT template that comprises at least a portion of the first integration recognition site, and
the first atgRNA and the second atgRNAs collectively encode the entirety of the first integration recognition site.

138. The system of any one of claims 120-135, wherein the vector further comprises a nicking gRNA.

139. The system of any one of claims 120-135, wherein the LNP further comprises a nicking gRNA.

140. The system of any one of claims 120-139, wherein the template polynucleotide comprises at least one of the following: a gene, an expression cassette, a logic gate system, or any combination thereof.

141. The system of any one of claims 120-140, wherein the template polynucleotide comprises a second integration recognition site.

142. The system of claim 141, wherein the second integration recognition site is a cognate pair with the first integration recognition site.

143. The system of any one of claims 120-142, wherein the template polynucleotide comprises at least a third integration recognition site.

144. The system of claim 143, wherein the template polynucleotide construct further comprises at least a fourth integration recognition site.

145. The system of claim 143, wherein the third integration recognition site and the fourth integration recognition site are selected from attB, attB2, attP, or attP2.

146. The system of any one of claims 120-145, wherein the vector further comprises a sub-sequence that is capable of self-circularizing to form a self-circular nucleic acid.

147. The system of claim 146, wherein the sub-sequence of vector that is capable of self-circularizing includes the template polynucleotide, whereby upon self-circularizing the self-circular nucleic acid comprises the template polynucleotide.

148. The system of claim 146 or 147, wherein the sub-sequence is flanked by the third integration recognition site and the fourth integration recognition site.

149. The system of claim 148, wherein self-circularizing is mediated by recombination of the third integration recognition site and the fourth integration recognition site by the integrase.

150. The system of any one of claims 146-149, wherein the self-circular nucleic acid comprises one or more additional integration recognition sites that enable integration of additional nucleic acid cargo.

151. The system of any one of claims 120-150, wherein the vector is a recombinant adenovirus, a helper dependent adenovirus, or an adeno-associated virus.

152. A system capable of site-specifically integrating a template polynucleotide into the genome of a cell, the system comprising:
(a) a lipid nanoparticle (LNP) comprising:
(i) a gene editor polynucleotide, and
(ii) a first attachment site-containing guide RNA (atgRNA); and
(b) a vector comprising:
(i) a template polynucleotide, and
(ii) a second atgRNA.

153. A system capable of site-specifically integrating a template polynucleotide into the genome of a cell, the system comprising:
(a) a lipid nanoparticle (LNP) comprising
(i) a gene editor polynucleotide,
(ii) a first attachment site-containing guide RNA (atgRNA), and
(iii) a second atgRNA; and
(b) a vector comprising:
(i) a template polynucleotide.

154. A system capable of site-specifically integrating a template polynucleotide into the genome of a cell, the system comprising:
(a) a lipid nanoparticle (LNP) comprising
(i) a gene editor polynucleotide, and
(ii) a first attachment site-containing guide RNA (atgRNA); and
(b) a vector comprising:
(i) a template polynucleotide, and
(ii) a nicking gRNA.

155. The system of any one of claims 152-154, wherein the gene editor polynucleotide comprises:
a polynucleotide sequence encoding a prime editor system.

156. The system of claim 155, wherein the prime editor system comprises a nucleotide sequence encoding a nickase and a nucleotide sequence encoding a reverse transcriptase.

157. The system of claim 156, wherein the nucleotide sequence encoding the nickase and the nucleotide sequence encoding the reverse transcriptase are positioned in the gene editor polynucleotide such that when expressed the nickase is linked to the reverse transcriptase.

158. The system of claim 157, wherein the nickase is linked to the reverse transcriptase by in-frame fusion.

159. The system of claim 157, wherein the nickase is linked to the reverse transcriptase by a linker.

160. The system of claim 159, wherein the linker is a peptide fused in-frame between the nickase and reverse transcriptase.

161. The system of any one of claims 152-160, wherein the gene editor polynucleotide further comprises:
a polynucleotide sequence encoding at least a first integrase.

162. The system of claim 161, wherein the linked nickase-reverse transcriptase are further linked to the first integrase.

163. The system of any one of claims 152-162, further comprising a second vector.

164. The system of claim 163, wherein the second vector comprises a polynucleotide sequence encoding at least a first integrase.

165. The system of any one of claims 161-164, wherein the first integrase is selected from BxB1, Bcec, Sscd, Sacd, Int10, or Pa01.

166. The system of any one of claims 152-165, wherein the gene editor polynucleotide further comprises a polynucleotide sequence encoding a recombinase.

167. The system of claim 166, wherein the recombinase is FLP or Cre.

168. The system of any one of claims 152-167, wherein the first atgRNA comprises:
(i) a domain that is capable of guiding the prime editor system to a target sequence; and
(ii) a reverse transcriptase (RT) template that comprises at least a portion of an at least first integration recognition site.

169. The system of claim 168, wherein the RT template comprises the entirety of the first integration recognition site.

170. The system of any one of claims 152, 153 or 155-169, wherein the first atgRNA and the second atgRNA are an at least first pair of atgRNAs, wherein
the at least first pair of atgRNAs have domains that are capable of guiding the prime editor system to a target sequence;
the first atgRNA further includes a first RT template that comprises at least a portion of the first integration recognition site;
the second atgRNA further includes a second RT template that comprises at least a portion of the first integration recognition site, and
the first atgRNA and the second atgRNAs collectively encode the entirety of the first integration recognition site.

171. The system of any one of claims 152-170, wherein the template polynucleotide comprises at least one of the following: a gene, an expression cassette, a logic gate system, or any combination thereof.

172. The system of any one of claims 152-171, wherein the template polynucleotide comprises a second integration recognition site.

173. The system of claim 172, wherein the second integration recognition site is a cognate pair with the first integration recognition site.

174. The system of any one of claims 152-173, wherein the template polynucleotide comprises at least a third integration recognition site.

175. The system of claim 174, wherein the template polynucleotide construct further comprises at least a fourth integration recognition site.

176. The system of claim 175, wherein the third integration recognition site and the fourth integration recognition site are selected from attB, attB2, attP, or attP2.

177. The system of any one of claims 152-176, wherein the vector further comprises a sub-sequence that is capable of self-circularizing to form a self-circular nucleic acid.

178. The system of claim 177, wherein the sub-sequence of vector that is capable of self-circularizing includes the template polynucleotide, whereby upon self-circularizing the self-circular nucleic acid comprises the template polynucleotide.

179. The system of claim 177 or 178, wherein the sub-sequence is flanked by the third integration recognition site and the fourth integration recognition site.

180. The system of claim 179, wherein self-circularizing is mediated by recombination of the third integration recognition site and the fourth integration recognition site by the integrase.

181. The system of any one of claims 178-180, wherein the self-circular nucleic acid comprises one or more additional integration recognition sites that enable integration of additional nucleic acid cargo.

182. The system of any one of claims 152-181, wherein the vector is recombinant adenovirus, helper dependent adenovirus, or an adeno-associated virus.

183. A system capable of site-specifically integrating at least a first integration recognition site into the genome of a cell, the system comprising:
(a) a first lipid nanoparticle (LNP) comprising:
(i) a first gene editor polynucleotide, and
(ii) a first attachment site-containing guide RNA (atgRNA); and
(b) a second lipid nanoparticle (LNP) comprising:
(i) a second gene editor polynucleotide, and
(ii) a second attachment site-containing guide RNA (atgRNA).

184. The system of claim 183, wherein the first atgRNA and the second atgRNA are an at least first pair of atgRNAs.

185. The system of claim 184, wherein the first LNP and the second LNP are mixed at a ratio of 1:0.25, 1:0.5, 1:0.75, 1:1, 0.75:1, 0.5:1, or 0.25:1.

186. The system of any one of claims 183-185, wherein the first gene editor polynucleotide, the second gene editor polynucleotide, or both comprise:
a polynucleotide sequence encoding a prime editor system.

187. The system of claim 186, wherein the prime editor system comprises a nucleotide sequence encoding a nickase and a nucleotide sequence encoding a reverse transcriptase.

188. The system of claim 187, wherein the nucleotide sequence encoding the nickase and the nucleotide sequence encoding the reverse transcriptase are positioned in the gene editor polynucleotide such that when expressed the nickase is linked to the reverse transcriptase.

189. The system of claim 188, wherein the nickase is linked to the reverse transcriptase by in-frame fusion.

190. The system of claim 188, wherein the nickase is linked to the reverse transcriptase by a linker.

191. The system of claim 190, wherein the linker is a peptide fused in-frame between the nickase and reverse transcriptase.

192. The system of any one of claims 183-191, wherein the first gene editor polynucleotide, the second gene editor polynucleotide, or both, further comprise:
a polynucleotide sequence encoding an integrase.

193. The system of claim 192, wherein the linked nickase-reverse transcriptase are further linked to the integrase.

194. The system of any one of claims 183-193, wherein the first gene editor polynucleotide, the second gene editor polynucleotide, or both, further comprise:
a polynucleotide sequence encoding a recombinase.

195. The system of claim 194, wherein the nickase-reverse transcriptase are further linked to the recombinase.

196. The system of any one of claims 183-195, wherein the first gene editor polynucleotide and the second gene editor polynucleotide are the same.

197. The system of any one of claims 183-196, wherein the first gene editor polynucleotide is mRNA, the second gene editor polynucleotide is mRNA, or both the first and second gene editor polynucleotides are mRNA.

198. The system of claim 197, wherein the first LNP comprises a ratio of mRNA to atgRNA of 1:0.25, 1:0.5, 1:0.75, 1:1, 0.75:1, 0.5:1, or 0.25:1.

199. The system of claim 197 or 198, wherein the second LNP comprises a ratio of mRNA to atgRNA of 1:0.25, 1:0.5, 1:0.75, 1:1, 0.75:1, 0.5:1, or 0.25:1.

200. The system of any one of claims 183-191, further comprising an integrase.

201. The system of claim 200, wherein the system comprises a polynucleotide sequence encoding the integrase.

202. The system of claim 201, wherein the polynucleotide sequence is encoded in a first vector.

203. The system of claim 202, wherein the first vector is a vector selected from: an adenovirus, an AAV, a lentivirus, a HSV, an annelovirus, a retrovirus, a Doggybone™ DNA (dbDNA), a minicircle, a plasmid, a miniDNA, an exosome, a fusosome, or a nanoplasmid.

204. The system of claim 202 or 203, wherein the first vector further comprises a template polynucleotide and a sequence that is an integration cognate with the first integration recognition site.

205. The system of any one of claims 183-204, further comprising delivering a recombinase.

206. The system of claim 205, wherein delivering the recombinase comprises co-delivering the recombinase with (a) and (b).

207. The system of claim 205 or 206, wherein the system comprises delivering a polynucleotide sequence encoding the recombinase.

208. The system of claim 207, wherein the polynucleotide sequence is encoded in the first vector.

209. The system of any one of claims 183-208, further comprising delivering a second vector.

210. The system of claim 209, wherein the second vector comprises a template polynucleotide and a sequence that is an integration cognate with the first integration recognition site.

211. The system of claim 209 or 210, wherein the second vector is a vector selected from: an adenovirus, an AAV, a lentivirus, a HSV, an annelovirus, a retrovirus, a Doggybone™ DNA (dbDNA), a minicircle, a plasmid, a miniDNA, an exosome, a fusosome, or a nanoplasmid.

212. The system of any one of claims 204-211, wherein the template polynucleotide comprises at least one of the following: a gene, an expression cassette, a logic gate system, or any combination thereof.

213. The system of any one of claims 204-212, wherein the template polynucleotide comprises a second integration recognition site.

214. The system of claim 213, wherein the second integration recognition site is a cognate pair with the first integration recognition site.

215. The system of any one of claims 204-214, wherein the template polynucleotide comprises at least a third integration recognition site.

216. The system of claim 215, wherein the template polynucleotide further comprises at least a fourth integration recognition site.

217. The system of claim 216, wherein the third integration recognition site and the fourth integration recognition site are selected from attB, attB2, attP, or attP2.

218. The system of any one of claims 204-217, wherein the vector further comprises a sub-sequence that is capable of self-circularizing to form a self-circular nucleic acid.

219. The system of claim 218, wherein the sub-sequence of the vector that is capable of self-circularizing includes the template polynucleotide, whereby upon self-circularizing the self-circular nucleic acid comprises the template polynucleotide.

220. The system of claim 218 or 219, wherein the sub-sequence is flanked by the third integration recognition site and the fourth integration recognition site.

221. The system of claim 220, wherein self-circularizing is mediated by recombination of the third integration recognition site and the fourth integration recognition site by the integrase.

222. The system of any one of claims 217-220, wherein the self-circular nucleic acid comprises one or more additional integration recognition sites that enable integration of additional nucleic acid cargo.

223. The system of any one of claims 183-222, wherein
the at least first pair of atgRNAs have domains that are capable of guiding the prime editor system to a target sequence;
the first atgRNA further includes a first RT template that comprises at least a portion of a first integration recognition site;
the second atgRNA further includes a second RT template that comprises at least a portion of the first integration recognition site, and
the first atgRNA and the second atgRNAs collectively encode the entirety of the first integration recognition site.

224. The system of claim 223, wherein the first integration site is an AttB sequence, a FRT sequence, or a VOX sequence.

225. The system of any one of claims 183-224, wherein the first atgRNA, the second atgRNA or both are synthetic.

226. The system of any one of claims 192-225, wherein the integrase is selected from BxB1, Bcec, Sscd, Sacd, Int10, or Pa01.

227. The system of any one of claims 194-226, wherein the recombinase is FLP or Cre.

228. A cell comprising the delivery system or co-delivery system of any one of claims 120-227.

229. A pharmaceutical composition comprising the delivery system or co-delivery system of any one of claims 120-227.

230. A method of treating a patient in need thereof, the method comprising administering an effective amount of the system of any one of claims 120-227, the cell of claim 228, or the pharmaceutical composition of claim 229.

231. A method of treating a patient in need thereof, the method comprising:
Administering:
(a) an effective amount of the LNP, the first vector, or the second vector of any one of claims 120-227 as a first dose; and
(b) an effective amount of the LNP, the first vector, or the second vector of any one of claims 120-227 as a second dose.

232. The method of claim 231, wherein the first dose and the second dose are separately administered by multiple administrations.

233. The method of claim 232, wherein the first dose and the second dose are administered at least 1 day, 2 days, 3 days, 4 days, 5 days, 6 days, or 7 days apart.

234. The method of claim 231, wherein the first dose and the second dose are administered at least 2 weeks, 3 weeks, 4 weeks, 5 weeks, 6 weeks, 7 weeks, or 8 weeks apart.
